SEQUENCE LISTING (1006 sequences)

SEQ ID NO: 1 - protein, 888 aa, synthetic construct
MKTKDIINTVRSSNIKFKSYDKELETFYNHLQDDYKKFFHLFLDWLCLGYTYINEDNQSVIVEPEEQLKQLKKYTKGNQKLEANIKAFVNLLTSGQTPASSCYNSFYANYSAAETSRCNKKLVEKLLESNKKQIEEYIESSKNALLKNISVDKWLEHKKELSKFKSISDKKKREKAQAEYWKEHIGQATLNTDRHCGAYLRKELKIKCRHPENASYCKTLCEMIRRQLTAVLEHNKQHAEELNKTKEQIEKFSQDESFKLICKIAETYPDDFHHYNFKNALNHIFYNKKMDSKNKDKIQLIVDALLPSKGLGYKTLFKAFDNWEIFERYQYRIKNRKYPLKPRLSDVNVPFDNFRFSDGSKFQGKLLNFNFECCYSHYFHDLQFEKIKEGYNLSFFHRLKNGNTKNVILTSNKISGTVKEVSVLKKNNELHLSVPYNIYHSNQNENLKYYFSFAAPSSEKLDLLPDSFKAIGVDLNIQDPVVVTLGAITKKDSYKIKVRDFGSAKLLETQTLVPDTNLSNEIKKLNSEIMTIKGILYEIKKISKDDSLEFDIESLGYLGLEIDRSKDHIGARGVVQNKIKLLKSKIKQYRHKCRNTGNKSVSELIRLIEIVDNFNSMISVYQRLHLKSGEQLLATKRNDNTRKNLKKDLLKKLANKIVKYCLDNQVSLVFLEDLDSEQSSRNSRERNSLIRLFSAKSILDSIETALGKYGISCLSNIDKKGTSKTDPFTGNIAYRDEKDKRNLYVNVKKKNKLVCYNADHTASLNVFLRGIDHSIIPYSFLWANSKDKDKEKGKRISRFLKDKFGTTKVYFYDLGEDHLTVSTKSLKNKSKYFGKIYYRNNKFITEKQHFEMEEKLKKEIMDGLAVGTQVEKIDITSKNINTCVNFQE

SEQ ID NO: 2 - protein, 997 aa, synthetic construct
MSETTKSKFPINKQVTNTVSADILADEKTTSKLTDILNVNSIVKNDLLKLMLTFRGGKCQLDDVEFDSDKEFDKLYAALGTSLPKEVIKALAKEFYYGLTGGLTSRAVKNIIYSPKEIKTSVSYKMDVPDQPNKKESKDKDQKKGKTSKKAKKPKKSVVTSEFDILRKSGIFPLKTPQECSGGMLYSVVKTVQNDLISLEEKTKAYEADKQCWLSKLATALCKFPHIGRYGDFLASVEGLSGIKSWNANGYIRFIKLWHDNSSRKGIRSFLLEKHTQYGNKSVAFTAEDTCGFGYQPDFFDSLYKNFKDLWLIEDVIGNEDYISFICGRDNTEDDKKNKGGAYHWRKEFINISSPERYGKKAVLMLSNGVDNDKGNFVPFTLDFSNENWIMTIDNPLTGEKLSFKLSKGTYYNNFSVLPNKPVKVKKDKNVKKDKNVKVEKAKGGYKIRYSTRKDDKYMYISYLKQPCLRYDPKKGCFKIDFINSDKQVEGLDVNVKNLRGMQKYFSTSSNFDKRAKDVLPGFENKEFIAIGADRGTFPTAAFAVARVNKSKDGTFTIKEWLDEEFLSRNENADEMTRKIKNVTDKITAVKRLTNETVKFKKGEIAEIPPTFTRTFKGKDGTVTATIKWQTLLEIDAEDYTGQVKKLPYDKDSKFTLLQQWIPSSVEWDVKRVMHDIKIEKNAIKNAFQNSENKTEYLQQNNKVDSSTYRKLEMIKGWISLQKKFCGSGPDVCKKDYDWLNGLRKHIDNQLSSNIVAVARKYREKYGLEVVVFLEKLDAFLSGFDDKHHNSTKALWGIGSLNKKLSEKGEKFGILFVPVDPYQTSIMDSETNLLGYRNWAEDKSDLYVKRNGEIQVVNADINAAKNIIIRGIFRHKNIVEFSSDEISDDLYRVMPVGKQRKGALFGHTGSDAILLSDKDGDGELFVASKSLNEAQQQATPINKDKKKVYVVFQDGKWIFRHKLDEQLKQYPSLLKAKPQVSKGNKKSVISRDLQLAH

SEQ ID NO: 3 - protein, 903 aa, synthetic construct
MSCVCNNSIPIDEEIYMALIQRAGVLKTKSDFPKVIKDWHDSLLADYRKFFPIIFSWCPEYGYTTIQDNKPVFVSPEERIESIRKEAKEHLNEVLAFGKMIGSKGVGGSSSYAIFYKHHKNNENGAYTPSRAKFMKEGIHNRRVELVDALMLNAIPDKEWIRIAQEVVGYSEDRLKLYWNKFIAKKVVSHDRKLGKIVREKYLEPKGLVCAQPENSTYCRVLTEIIKRQLHSQIEKSKFHEEELKSIEKTVSEFDSPLLDVICQYAEELNQINSGLSKYVIKNAVKEVISPPEKQSEIYVQSQVLSQEKYKPLVNATIKEILSGYEQWKVKSRYENRLKNRKYVLYPKLSANYKIPIGQNSLGRFKINVSENGEIVIRLNNMADVVCMPSKYFFNLKSSPVVDKKKQLVGYQISFNHNSRRKEPTEKPDFNGIVKEIGLQLKDDGRFYITLPYCMEYSNDNFDLIRPLLTSSPTEDQIKKMPSEFNVVGFDLNLSMPLPITRAIVGKSVKGEINVEYLGQAKVIESTHLIYDNNRCKVLIAYKRQCDLIKRAIREWKICKGKNIDISEKTYEWLESHTKRWNPSRQPESMQDRFSVSKMRIQILVNKAKSRIAKYNDNSWKTGHGNESELIRLIDADDAYNSLVSTYNRIHLKSNQFIYALPSKNNSRSNKKEYCLRRIAAKIARYCHLHNVNICIGENLSFQQDSDNISKDNSLVRLFSSKSIASYMKLAMEKFGIAFIDSADPSGTSKTDPVTGNIGYRNKFDKRKLHVLRNGNWGWVDSDIAASLNILIRGINRSIVPYKFFVGKKKQESKRLNHFLNKIFGTTKVFFYEDQFGFANPSLSKKEGENLIANQYLYYREGKFVTQKIHRQIEDDFKKIDFSNTPEVNLIPSGVKLKNFQFE

SEQ ID NO: 4 - protein, 907 aa, synthetic construct
MATLDAMPAIHGFDSTSRCYMALIQRAGVLKTKSDFPKVIKDWHDSLLADYRKFFPIIFSWCPEYGYTTIQDNKPVFVSPEERIESIRKEAKEHLNEVLAFGKMIGSKGVGGSSSYAIFYKHHKNNENGAYTPSRAKFMKEGIHNRRVELVDALMLNAIPDKEWIRIAQEVVGYSEDRLKLYWNKFIAKKVVSHDRKLGKIVREKYLEPKGLVCAQPENSTYCRVLTEIIKRQLHSQIEKSKFHEEELKSIEKTVSEFDSPLLDVICQYAEELNQINSGLSKYVIKNAVKEVISPPEKQSEIYVQSQVLSQEKYKPLVNATIKEILSGYEQWKVKSRYENRLKNRKYVLYPKLSANYKIPIGQNSLGRFKINVSENGEIVIRLNNMADVVCMPSKYFFNLKSSPVVDKKKQLVGYQISFNHNSRRKEPTEKPDFNGIVKEIGLQLKDDGRFYITLPYCMEYSNDNFDLIRPLLTSSPTEDQIKKMPSEFNVVGFDLNLSMPLPITRAIVGKSVKGEINVEYLGQAKVIESTHLIYDNNRCKVLIAYKRQCDLIKRAIREWKICKGKNIDISEKTYEWLESHTKRWNPSRQPKSMQDRFSVSKMRIQILVNKAKSRIAKYNDNSWKTGHGNESELIRLIDADDAYNSLVSTYNRIHLKSNQFIYALPSKNNSRSNKKEYCLRRIAAKIARYCHLHNVNICIGENLSFQQDSDNISKDNSLVRLFSSKSIASYMKLAMEKFGIAFIDSADPSGTSKTDPVTGNIGYRNKFDKRKLHVLRNGNWGWVDSDIAASLNILIRGINRSIVPYKFFVGKKKQESKRLNHFLNKIFGTTKVFFYEDQFGFANPSLSKKEGENLIANQYLYYREGKFVTQKIHRQIEDDFKKIDFSNTSEVNLIPSGVKLKNFQFE

SEQ ID NO: 5 - protein, 831 aa, synthetic construct
VVTPESQSDAKKAFADALFAGQEAAYGSFYAKYTPLPFPRKDSALGRLTAELGTIGDEKYAKALKLNGLDPDEWAGYRNRAIAGEPPDALWEEIKVKVGVTNKATLASRGVGNLPTLRPDSKSYATALAYMLSSSLSSWEECAKLHRARREEIAAKAAVPDSMTEEYALVCQLAQALEEAGSGLTAKMLNGAIREMAKTDSVYMVAITELADPKYKPLLALDKEDRARLMDTWQAERKLSTSLAAPKRPLFDKDYQIPFGMTGRGESFALRTDDSGRLWVKIGDHEEFSCAASDYFGNLKVEALPSDKGYKITYRKMVKNRRRQRAGYKSSVGGPLIEGEVKQILLQRRMNRRGKESFYVYLPHSIAVKDDRRHLSWFFKRADWKDGKGLEELPDQLIVSGIDLNIDQPVTVTKAIVSKNYTEGEINCPYGGGHLTGKPRSVCPDTPISQEAKLTHRKMRLLIEVIRESKACHKAGTPLSSEGSERLKILYADISKPPCRNNRESIQVVYSSLKKTYRRLKYELMQQGANDVSGCIRMLQLDDTHRALTACFDAFSGKPALAGRNFGVARKKTSKGTARLNFREHVSHLYANAVLRACHGSDVVFIEDLSTSFDGNAHNSLKRLFAPGQLIDAITSLLESHGIAVVTVDPSGTSRRHPITGDIWYRDSMKKRELVGLIDGELVSVNDDLAASINVLTKGLNHSVVPHKFKVDPSGRVNVKEDGGKRLKVFLREKGLSGRLPVGSADAAKSKKAKALTVYPSSSGLLSDRQVFEVLEQIKHLLAAGCKPRPVELMAGPGETYAAFSLKEVLEWSKSKSSEDLRHTGLGDETG

SEQ ID NO: 6 - protein, 882 aa, synthetic construct
MEKFPRCSHLKVKQHCGPFVEWYEDLQSDRQKYVSLFFPWIGIGYGERKDEGDDIDFISPEQTLERRKAELGKLEELVVRFVTVMLKGGSSASSCYSLFYEDYKGKDTAKKSTSKVNFIKALPLLSKKEIEDRVLGDNQLSLINLDEWMELGQSGLSADDIWDKVSQRLVTGERSLGVDVRHTLKIRCQRPRDRGYCVVLMEMVARQLRSCYECYKNHLNELQELREKTTKQITESRDDLFSLVCDLATVLTEQDSGLSERTLFSGRKQALKPQGKPSKIQAIVDELRQPQYSPLLEADDKELRQAYKAWNSQRRLRDRETRRPYPCRPNWRNDYQVSLGLTGRGGFWMWMDGSDVVVDLQDCGRLTCSPSHYFGDLAIEGQLHQYDIRYRNKLKRRGKKLRKPPAYGEWIEATIKEIGIQRKGGDYYVYLLPVISHDSTNFQAAKKFFAAAAPKQEIIDALPDEMKVLALDLNISNPVCSVKARIGRALDGPLSVLDYGQGEIVDGPKILVTDGPRCVALIELHSDVFFVKDAIREYKRTHGVAFNGAGMDDEMLERLSSIPSPSPSVRCQIQAWVAAIGKRLHLLHYGMRREGYKNLGEAIRLLAAMDAYDSMLQSYQRIHLKKGQSLPRQAKNDHSRAGLRETITRRIAYAIAQEARDCNVVFVEDLKADFDADNDNNALLRLFAVKTLIRYIQEALEKIGVGLVLVAKDGTSKTNPMNGDLGWRDRNNKEHLYFELNGEAVRCHADLVASLNVLLRGLDHSVCPYSFVTKDTNKKPGRADQKDKGYGKRLKRFFKDRFGSSNLRFVAKPDGEVTATTRRPKDALIGERVYYHGGKLVTSEVHYRMENEIKHLLTLKSPAKELSLTEDGIRSYENFTHV

SEQ ID NO: 7 - protein, 882 aa, synthetic construct
MEKFPRCSHLKVKQHCGPFVEWYEDLQSDRQKYVSLFFPWIGIGYGERKDEGDDIDFISPEQTLERRKAELGKLEELVVRFVTVMLKGGSSASSCYSLFYEDYKGKDTAKKSTSKVNFIKALPLLSKKEIEDRVLGDNQLSLINLDEWMELGQSGLSADDIWDKVSQRLVTGERSLGVDVRHTLKIRCQRPRDRGYCVVLMEMVARQLRSCYECYKNHLNELQELREKTTKQITESRDDLFSLVCDLATVLTEQDSGLSERTLFSGRKQALKPQGKPSKIQAIVDELRQPQYSPLLEADDKELRQAYKAWNSQRRLRDRETRRPYPCRPNWRNDYQVSLGLTGRGGFWMWMDGSDVVVDLQDCGRLTCSPSHYFGDLAIEGQLHQYDIRYRNKLKRRGKKLRKPPAYGEWIEATIKEIGIQRKGGDYYVYLLPVISHDSTNFQAAKKFFAAAAPKQEIIDALPDEMKVLALDLNISNPVCSVKARIGRALDGPLSVLDYGQGEIVDGPKILVTDGPRCVALIELHSDVFFVKDAIREYKRTHGVAFNGAGMDDEMLERLSSIPSPSPSVRCQIQAWVAAIGKRLHLLHYGMRREGYKNLGEAIRLLAAMDAYDSMFQSYQRIHLKKGQSLPRQAKNDHSRAGLRETITRRIAYAIAQEARDCNVVFVEDLKADFDADNDNNALLRLFAVKTLIRYIQEALEKIGVGLVLVAKDGTSKTNPMNGDLGWRDRNNKEHLYFELNGEAVRCHADLVASLNVLLRGLDHSVCPYSFVTKDTNKKPGRADQKDKGYGKRLKRFFKDRFGSSNLRFVAKPDGEVTATTRRPKDALIGERVYYHGGKLVTSEVHYRMENEIKHLLTLKSPAKELSLTEDGIRSYENFTHV

SEQ ID NO: 8 - protein, 994 aa, synthetic construct
MSETTKSEFPINKQVTNTVSADILADEKTTSKLTDILNVNSIVKNDLLKLMLTFRGGKCQLDDVEFDSDKEFDKLYAALGTSLPKEVIKALAKEFYYGLTGGLTSRAVKNIIYSPKEIKTSVSYKMDVPDQPNKKESKDKDQKKGKTSKKAKKPKKSVVTSEFDILRKSGIFPLKTPQECSGGMLYSVVKTVQNDLISLEEKTKAYEADKQYWLSKLATCKYPHIGRYGDFLASVEGLSGIKSWNANGYIRFIKLWHDNSSRKGIRSFLLEKHTQYGNKSVAFTAEDTCGFGYQPDFFDSLYKNFKDLWLIEDVIGNEDYISFICGRDNTEDDKKNKGAYHWRKEFINISSPERYGKKAVLMLSNGVDNDKGNFVPFTLDFSNENWIMTIDNPLTGEKLSFKLSKGTYYNNFSVLPNKPVKVKKDKNVKKDKNVKVEKAKGGYKIRYSTRKDDKYMYISYLKQPCLRYDPKKGCFKIDFINSDKQVEGLDVNVKNLRGMQKYFSTSSNFDKRAKDVLPGFENKEFIAIGADRGTFPTAAFAVARVNKSKDGTFTIKEWLDEEFLSRNENADEMTRKIKNVTDKITAVKRLTNETVKFKKGEIAEIPPTFTRTFKGKDGTVTATIKWQTLLEIDAEDYTGQVKKLPYDKDSKFTLLQQWIPSSVEWDVKRVMHDIKIEKNAIKNAFQNSENKTEYLQQNNKVDSSTYRKLEMIKGWISLQKKFCGSGPDVCKKDYDWLNGLRKHIDNQLSSNIVAVARKYREKYGLEVVVFLEKLDAFLSGFDDKHHNSTKALWGIGSLNKKLSEKGEKFGILFVPVDPYQTSIMDSETNLLGYRNWAEDKSDLYVKRNGEIQVVNADINAAKNIIIRGIFRHKNIVEFSSDEISDDLYRVMPVGKQRKGALFGHTGSDAILLSDKDGDGELFVASKSLNEAQQQATPINKDKKKVYVVFQDGKWIFRHKLDEQLKQYPSLLKAKPQVSKGNKKSVISRDLQLAH

SEQ ID NO: 9 - protein, 887 aa, synthetic construct
MALIQRAGVLKTKSDFPKVIKDWHDSLLADYRKFFPIIFSWCPEYGYTTIQDNKPVFVSPEERIESIRKEAKEHLNEVLAFGKMIGSKGVGGSSSYAIFYKHHKNNENGAYTPSRAKFMKEGIHNRRVELVDALMLNAIPDKEWIRIAQEVVGYSEDRLKLYWNKFIAKKVVSHDRKLGKIVREKYLEPKGLVCAQPENSTYCRVLTEIIKRQLHSQIEKSKFHEEELKSIEKTVSEFDSPLLDVICQYAEELNQINSGLSKYVIKNAVKEVISPPEKQSEIYVQSQVLSQEKYKPLVNATIKEILSGYEQWKVKSRYENRLKNRKYVLYPKLSANYKIPIGQNSLGRFKINVSENGEIVIRLNNMADVVCMPSKYFFNLKSSPVVDKKKQLVGYQISFNHNSRRKEPTEKPDFNGIVKEIGLQLKDDGRFYITLPYCMEYSNDNFDLIRPLLTSSPTEDQIKKMPSEFNVVGFDLNLSMPLPITRAIVGKSVKGEINVEYLGQAKVIESTHLIYDNNRCKVLIAYKRQCDLIKRAIREWKICKGKNIDISEKTYEWLESHTKRWNPSRQPESMQDRFSVSKMRIQILVNKAKSRIAKYNDNSWKTGHGNESELIRLIDADDAYNSLVSTYNRIHLKSNQFIYALPSKNNSRSNKKEYCLRRIAAKIARYCHLHNVNICIGENLSFQQDSDNISKDNSLVRLFSSKSIANYMKLAMEKFGIAFIDSADPSGTSKTDPVTGNIGYRNKFDKRKLHVLRNGNWGWVDSDIAASLNILIRGINRSIVPYKFFVGKKKQESKRLNHFLNKIFGTTKVFFYEDQFGFANPSLSKKEGENLIANQYLYYREGKFVTQKIHRQIEDDFKKIDFSNTPEVNLIPSGVKLKNFQFE

SEQ ID NO: 10 - protein, 824 aa, synthetic construct
MESIRKEAKEHLNEVLAFGKMIGSKGVGGSSSYAIFYKHHKNNENGAYTPSRAKFMKEGIHNRRVELVDALMLNAIPDKEWIRIAQEVVGYSEDRLKLYWNKFIAKKVVSHDRKLGKIVREKYLEPKGLVCAQPENSTYCRVLTEIIKRQLHSQIEKSKFHEEELKSIEKTVSEFDSPLLDVICQYAEELNQINSGLSKYVIKNAVKEVISPPEKQSEIYVQSQVLSQEKYKPLVNATIKEILSGYEQWKVKSRYENRLKNRKYVLYPKLSANYKIPIGQNSLGRFKINVSENGEIVIRLNNMADVVCMPSKYFFNLKSSPVVDKKKQLVGYQISFNHNSRRKEPTEKPDFNGIVKEIGLQLKDDGRFYITLPYCMEYSNDNFDLIRPLLTSSPTEDQIKKMPSEFNVVGFDLNLSMPLPITRAIVGKSVKGEINVEYLGQAKVIESTHLIYDNNRCKVLIAYKRQCDLIKRAIREWKICKGKNIDISEKTYEWLESHTKRWNPSRQPESMQDRFSVSKMRIQILVNKAKSRIAKYNDNSWKTGHGNESELIRLIDADDAYNSLVSTYNRIHLKSNQFIYALPSKNNSRSNKKEYCLRRIAAKIARYCHLHNVNICIGENLSFQQDSDNISKDNSLVRLFSSKSIANYMKLAMEKFGIAFIDSADPSGTSKTDPVTGNIGYRNKFDKRKLHVLRNGNWGWVDSDIAASLNILIRGINRSIVPYKFFVGKKKQESKRLNHFLNKIFGTTKVFFYEDQFGFANPSLSKKEGENLIANQYLYYREGKFVTQKIHRQIEDDFKKIDFSNTPEVNLIPSGVKLKNFQFE

SEQ ID NO: 11 - protein, 903 aa, synthetic construct
MNHFTQNLAVTKIDSSVQTLYDDFQEDFRKWYSILCSWAKVPYRVEDDNGNITHVHAKQRAAELSSLYSAQEQLLIERFMRVFLGASKEASSSYSLFYDKYAPNGAKKTAALKEFLGEIPTLDDSQVEKKANTNKNCKRITVKEWRDFAAQFNKLGIDAIWGKLKEKITSPDRGVGLQVRGFVSERCKHPENAGYCRQLCQKLTEQVKSTVRICELYIQETADLQSEIGNKTKQPEVKKCFDLLCQLSDELRDVNSGLSGRVLFQSIKDIQGGKDSDRKNKVCRTVMSDDKYLCLIDSDKKIVQRAFQAWKLSITLKKRQEKLYPKQRSHQNDVPMPFGLTGLASFDVSPDGSKFTFSEGSVETKPSLYFDGLRFQKNKHSFDMEYRVAYASRRTKKGRRPKRVGRLRKATLKQVELFNKGGKFVIKLPHGIELPEHRAKLATFLMCADPKEKGYSKGGLVDEFRALAIDVNLSNPLAACVADVSKHGDGPIQALDYGRAKLCVDPFFLVANTPVCGQLIKLCKECKAVVQAIKEFKECQAAGSDFSFETDLFLPAGHSPRHRIQSKVQELSRVLRDWKFKLQNTNQVAEMIRLLELMDAYQSMISSYQRIHLKAGQTLPPQKKNSTKRANYRKDISRRLAFVVADYAQSLNVDVVFGEDLKADFDRDKNTNSLSRLFAGGMFLQHLENALAIRGIAFVKCAKNGTSITDPVSGVFGWRDKFNKERLWIERDGELGWVNSDHAACLNVLLVGLMHSVVPYRVYVKAGRKTTDDVEVFEEDHPIGPRKKRFFELCFGENKPKFEVVENRVILDNAGTYQGFIYAHCLDAMQSEKQHWAEYDRIKELALAETNPKQFGLTPKKGEGYRNFCQPDIHEQVEVVGNKRVAATKQILESLADEKIEAT

SEQ ID NO: 12 - protein, 872 aa, synthetic construct
MAYPPFVRTQNFKIKNITTNDELLQWYEDLQFDQRKFYNEFFSWTGLGYYVLAENDEIIYTSPEEAAIQRKEKLGDNESCVARYVDLILQGGKSASSCYNVFYENYKSTGKAKFSEMKCEFLKNLPFQTKKQIQEKISKNKQLSCLTLNEWWGIRDKIKTINKLESNEEKEIETKKVWNTISSQIADTDQTIGVEVRELLNIKCRRPNDCQYCKILSDMVGRQLRSFSEVTKNHLKGTDELKQKLNDSKNDLYNIICEYSDALDALESGLSKYVLFQAIKEAEKPNPESKIQLAVNEIKKGKYESLRQASRKELMKAWKALKLKKRLQIRKKYPRFVVGGNDYPINVGLTGKGNFRLSTHNNKILIEINNFGTFECYPSCYFGNFQATPIMRNKCICGYNIKFRNINKSKNSVTYGPWVIGVVKEVGIKRKNGNYYVSMPYTMRPEDTNFKIATYFAKAKPTLDEFEALPNEIICAGYDLNISNPVVQTTGRVVKNGHGPLKALDYGAGHITKIPTILTPNGLRCHRLIQLKADVYKTKDAIKEYSGNLKQHQDVSNETKEWLKSSDHNNTRYLIQKKIGSITKRFKTLAFEMRAEGYQNLAECIRLLDAMDARNSLLNSYKRGVQKLPPKRQFDSKRADFRNYINRRIGYLIAKSASEENCNIVFLEDLESDFDRDNNNNSLMRLFTPNTLIKCIEEALEKKGIGLVTVAKDGTSKTDPITGSPAHRDRYNKEKLYVERNGQIGWVHADESASLNVLLQGLNHSVCPYKFYVKESTKDYGIRMQRFLKERYGTTNVSFVEDNGQVEAVPKKRPANRLQNTYVYAHHDGIMVTKDKHLQIERKIEDSVMQQIDLPKFSLTDCDERGYRNFSV

SEQ ID NO: 13 - protein, 464 aa, synthetic construct
MPRNSKKKKQSEVRTLRLDVLPVRFGLHNAERVKYPAETTDEAKARVGEACNTVRTYIRKYRLAIRKACAPLLFAQAATEEWSDEKGRAVIGKADAVTEEMSKLTGDKVCIQRNLDRFVKALIPTLKCECFQQVANRIYTAITAKDPDIPISRHLLILDGVRKLPWFMNEPIHLKYCPTYRSDVIFNGHRVFLRWDKELGYVEFKLIGKKLKQNAKGNSKRGLDASQWYIIKQLQSGAWAPGGAKLQLMKDGDFRLICGYQGFELDRPELRAKAGRERIELIPVDKDRIDELSFTEDRERFMVLQLSSGHRASAGIPVDEVTKHEWSALGMLAHVDRLSKQQDRSKKRTSSCGAFHKAREAEQARRDRITRARENAVANWAHEWSLQVANRCMAMRCGQLRVFDIPKEVLFFRPFPWSKFRFDLEYKCRERGIEVSYHESPKDSKGSSDPNAEDSATASVDGQE

SEQ ID NO: 14 - protein, 617 aa, synthetic construct
MSETKEVFEAKKKLEEQEPTAEAVVALGKLLLAAEEEIGLNAASELLPKYLRKRMQLLLSAATGGKAEIEAALEPFVLDWHKKAINSAWKELREVKLERQLIQLARTLQEIDGRIGRQKLVSITPKRLLQQRKLDTLLTVASGLESAEEALEKVVSEPRPLRLQVRPIRVGLERPEHPKKIKNETAEEAKARVGAACEKLRAYLRKYRFSLRQAMNVMYWAQARTEKWNEEKGVPRVGSVEEVKAELRKMTGDPVSIVRNIDRYVRTMLPRINANCLQMASARMYVAWTSKMPETGIPRNLAVMDAAARMPWFANEPLHFKCFKSLNSDVKFDGHRIFVRFDHDLKWVELKIINTKHKRTYNEKPPPKGKRNGGKKSLDYMYWYVLKNLQKGEYSPAGARLQMKDGKFSFSCCYNSERGRPELNDGKKRPFRKVSYDRVADASFTEERTSFIKLSLREGHEPSAGIPVDQVREYPISAEAAIAAIDRLDAQQRKAENRIAATGSSNKRRKAEQKRRKRITEHRENVINQYCHDWSRQLADWCSVMGCGRMMVFDVPDLLFGRSFQWAKLRFHLQSKCRERGIEASFHESPKDSTGDTSRSGDPQAEVSAPCSVGLDE

SEQ ID NO: 15 - protein, 517 aa, synthetic construct
MEVKVLTKVKQPRKEGRGGGSILAARNDLVLKSDSLNVLFDGDLSHFRGVLSKWRCMIQEMENLVEIVEKAVADFSMKKKVVSEIDDPKHPGEKKKVSTWVPTDYVIVRMKLDDAKQAATSILGKDVNLCETKYQEWYRKWQELDVSMGGPHFDRRMFEQAIKIMGAIRNKKVPDRGNIPRNHLVVAGAAKQPWFGNVPLECIYASQSNVFRAKFVKEGNIRKVVLMLNDKDEDAKLTFVVRGAIVDKNGKPHTIGTKKSDRVFDNYFEGRWIPTNPRANIDKRGYCHLYVSNYRPRRAFTGLLDANTLEVTFHKINGEDIRKPWRPEKEQDRLTGRVFTIHTRIMRGNKRSWPANIAINDAIAAMRGYSKRMDEKRECWNTRRFGPKRLRRPFQEIIDNISKAKERTQRQINHRWTNEVIFQATRKGRCRTIRLYGFPDGVKAGLLLDGTVQWPWSEWLRMLIYKAEEAGCFIELVTTQEDINAMGVALHEELTRVKHKEEEEEDDDEAEYGPEAA

SEQ ID NO: 16 - protein, 844 aa, synthetic construct
MEFTNVVSKIREGIFHISMSTSPEITNKHRPLAMARSGSLTFSGNTVTFKGETHTVETPLTDERMPVHVTLTNYNGKVYEFPRSPNVDGLFAVKQKNGDFFLRGSNRNRGDKTEKEQGNYLTVKGRVYHHHRGVETSAELPAWAEAIRTRWKQGWNVLALTLRWVLWKTEDCGETEQDWEALIKNKVDHLLNSREGYVQPDALSPESTAIVDRIRRVIVDSSMAHYATRGREAKLSIPIVLRDNSKVPPAFSDLRNFAYQLGRYEKEEGVKVQMVDEGGEIDILSEWLGIELKALKRNKAPLKAFKALLKAKGEVWTDGFMGTLFGRIRAHLGMDALEAEALSTRFTAVLKQRESKSMPFDRGWPRPIRTGEDAWQLVYNLKGKTTLGELMNNKCSDIQFTQPVSSEISGSPNNKVVTKLKDGTFDVTNEYKPLTPGSVASQRELRGLSFTKGKKGMEPYKLAVLMHRNDVDLNAKVKRVVLSIKTEIEPYVIYKGQPTPSLGAASAFTSPAGRKVTFYQLNICLEGVKTNEKYDRRGTGASHLKLGWKVNGDQLTVVTETRPTGVIVPYSLPLGEVWKTEGILDAGCHSDKPGNAWSRRNNYKMAFYALNGWPVSPLVIEAQKQELVSRFIEGRLTLDWLKTTSLSLPKQIDTLLNVAKAEGFKLMTGGQRAIVTREGWHMVHQKGLVRRIKELRMAENGEAYPASNSNPSRSKEPSKPLGKNKELLTLWLRWLEYNDRLRAVERMVRGHLLGRRKAYYYALAHHLCDHTAAIQYTKLNLKDQAEKKTRKKKDKGEKAVVDNSQKYRMWASLGEFTTILKQVGIKRGVEVKEIEIALTKVAAA

SEQ ID NO: 17 - protein, 629 aa, synthetic construct
MKTKTKTATKTATKTHIDLVLRARLAERGSAGSAVRREARIVIFGADQQTPHPDSEKIVEDDLRGTHEYYNRLVRAVSPKYKKARGLVVGPEARKQELREKQISALYQERHDCAKSEEARINEEILKLKQERRESWALVDKERTALRKKNAEKIQDLIGPSTITPLIKAERNRVSVYGKTRQSSDPTIGWGNYTLAEKDFGLATRKAFNLKSIPHFRGEDRDGTAPEDFRAYKFWQKDGTLRSDFTQKNHHAHRTPEAIFGGKGKLMTIRPFEESDLAAFNLPAAFWDQIARHRSEGKPDRWYVILIVRNGTKVPDADWYGVVANFPAGSKQTPSLLDRLRSVDHVTEARIVRKKQGLRLETHVNLHVKIAVAAKVARETVEVSWGSFTNKGVGKLLVTYPDGRVDEFSEANVPALLRCRQHEETSRKLHESLSKHEKDARKAFKTLNLDPSIIWDREEFPRSPGSIRHVYRRLFRDEMVEALGGEAAREKRDEIMRLCLKLYRQPGPDGEKNPYGGDPDQAREIVAMRMAEIFPISEDKAAAMAVVFVFMERFNHLADWAHRSGFRYHTSRREFYRTFAAELCQKYNVTYLRPPKAGRKVSGDRSVVAPFVLETILKTAALKMGRAWE

SEQ ID NO: 18 - protein, 437 aa, synthetic construct
MSISDIFKANQFKTEIEELKRECNRLQSLLTPEMQDAAQLKREIDKLNFDKYNINNQINDLDKTLDAKYDAIKKLDIVINNKKSQIIWMDDEILVQEFGLYQPKFEFANSQQYKEKLNNNRKLQKESIKNCTAVTGSTNWTVDGNKSKGNKMVKDMQKLLLRAFNGECDELIGKVKYNNYDTSLKRITASKDAISKLGTIMGISITDTYFRLKKEELNLALEYQLKKQQEKEALAQARAEMREAVKLQKELDEQRKKIEKEQNHYQNALHSILKQLENAKDNEKEALETKRLELSKELENIKTSLKDLDYREANQKAGYVYIISNIGAFGENIYKIGMTRRLDPQERIDELGDASVPFKFDVHAIIFSDNAPALEAKLHKAFDSKKVNMVNQRREFFNVSLDEIKEVIRKNYDKTVEFIDVPEAEQFRISQKMKEQL

SEQ ID NO: 19 - protein, 425 aa, synthetic construct
MLEDNEFSKFAFTTHMKKYYKWCRSNISSMVAQAISENVWRAFEKYFYSNGKRLHFKKIGSVTTVAGKGGASGPREIRFKDGYVLWNGLRLKVKLDESNVYEQQMLEHRIRFCRILKKPDRRKNHYYVQFVLEGRPEIKIRPDTGEALHPVGVGRVGIDIGPQTIAYAAECGAGLRELAEGVGNIEREKRLLQRKLDRSRRATNPNNYNADGTIRRGVKLTKNKSNRYLAVQRELAYVYQRQAAMRKQKHIALANELLALGDTFFVEDMPWKGLAKKAKKTEISPKTGKYKRKKRFGKSLGNKAPATLIMILDNKLKSLGLDGVQKIPTTARASQYNHITQTYTQKTLSQRWNTMEDGTKIQRDLYSAFLLQHINEDNKTFNQQTLEEDYPAFQRYHDRVIVQLQNNPHNVASMGIRSATHKRAE

SEQ ID NO: 20 - protein, 416 aa, synthetic construct
MGIIKAAIQSATSTLADQWLDIIEPDTMDNTTLMCKGVLMSKNKKRGSNTKGSVDYISDGSIIRVYPNMMMLLVDGGQIIDYTAEEGYYKVDNSAAPSLFNGDFEGALNESFERFRFGGVTPSKQEVFFINLQEIRGIRFGTQQPINYFDNFYNAELFVRAHGSYSIAITDPILFFKNVVPKGVTRLTIDEINEQFLMEFVSGLQSSINQLSAQGERISYLASKTAELSKVMNQNLDEQWKSLRGIEIVSIALASISYTDDSQQLINMRNKGAMFNDPNLRESFMQTSIAQGIADAGNNPNGAANAFFGMNMGANLTGDYLSQASRQNQAQTQQAAQKQQTANTSSAQESSWQCPNCNQENTGKFCSNCGTPKPTPTANTGMKVRCSKCKSVIEITNAMPKFCPECGQPFLAESLD

SEQ ID NO: 21 - protein, 416 aa, synthetic construct
MGIIKAAIQSATSTLADQWLDIIEPDTMDNTTLMCKGVLMSKNKKRGSNTKGSVDYISDGSMIRVYPNMMMLLVDGGQIIDYTAEEGYYKVDNSAAPSLFNGDFEGALNESFERFRFGGVTPSKQEVFFINLQEIRGIRFGTQQPINYFDNFYNAELFVRAHGSYSIAITDPILFFKNVVPKGVTRLTIDEINEQFLMEFVSGLQSSINQLSAQGERISYLASKTAELSKVMNQNLDEQWKALRGIEIVSIALASISYTDDSQQLINMRNKGAMFNDPNLRESFMQTSIAQGIADAGNNPNGAANAFFGMNMGANLTGDYLSQASRQNQAQAQQAAQKQQTANTSAAQESSWQCPNCNQENTGKFCSNCGTPKPTPTTNAGMKVRCSKCKSVIEITNAMPKFCPECGQPFLAESLD

SEQ ID NO: 22 - protein, 1125 aa, synthetic construct
MAKRSVTPLTEDRQLWEPLPPDDVRLAFVKYLDRRPGSIRRTIQLQLHNPGWRKTQNLLWARKHFNNYIFTILETLREDPHQFRDDGAFECIHEMMVRREETRRQGKPPGKYTRVLGEVIAERFRRPARSGPNNQDTNDETVADNASDRRNHPSRRRPPLRILEARDYPIIHMLRPGAQRRCALILLNFYIQSQKIPQAHEQAPDSQSDEASQNTASNRELEHAHDASHSTAAADFSDEEAAEREETLEAEMEALETDSIPTIEMMHQMWAQRWMRYEAAASTRIPYGQHRGKPFGEALAARRMRTGRERGRLVRKPGKRRVRWLKERTYALEQVHTCLRAVQILLHRDTYGNDSAEVLEAEQVRHLWKYESRTLRRPRARRVEHYRSRQSQPLATLGSLSQEELHTFRDELYEQLDHAHQYAQIAEAVDIFLEHAPPSYPTIDIGLFNDSPERREHHSALNWFPRPVTRGSIRDSDDDIERRPGPSVDELLHRAEYDAVTRLLQAESELGERALQPLTYTRTMRPPAPLPCWALLYKRSCPSHEEREQCARRLPKRMRQRYKDTALNSTDGYVYTFVLAMVVHHTTHAFTGEQPADYFSPSLSEDEYFYVNFPLTPFHPPQGVSLMMFPLECGEEYQEGLLHDLIERLRAEQHAQYAHQFTDPDVDPADRVPVEACLPTQAALGEAKIVTRQNGDDWYEFFLHCAVPIDILVREPPERILGLHHHQHGYSWALIDAVGTLINAGDLQIPWHVRLRPGSSAYSDNYAFEVANQIVALAQEQNALICIENTWTNKKATTSAHQNQRAFAHPSRKIYDQVVSKAIEAGLPRPRAVYGISRSRCGACGNRINNQRGKETLSACPACGSLSRSAHIQFPLASLAPTADGSTAAWVLLAQEQEHGERAAALTDFFYWTLRQAAADVVPAPDRRQAFIAGLEQVQWQGRQVFRPKSGRNTYTRNNQQAGTNSAPSYTFRPRRLEADVVIAGPEAYAPRVQQWIAAYELVAPDVLIEYVSMLREHALDILYGGREDAVADVILLDTPASERELAAGLAQQRSWFCQDCGQHWQVDELRFRCTYEPCRHHDLARYNTAAVVAQIAVHKLLEGSQQERQHATPGTTGGEHETLA

SEQ ID NO: 23 - protein, 465 aa, synthetic construct
MEKYNVSISKSELMSKQDFFKLWMRAYDGAEEVEENHWMTRFDNIINNSAKRIPRYKEKIAQIVFKVLEESYPFFIWGIPNEEQGDFIKTCEKDIKNATKKTISIIIAPCTSLSDFCAKYVRLQLMGGQEWLDMFASNISHYIPTYYAANYLQMPKSEEETLSSFFKGSAITVDNESVSFASLFERELQTACFVYLAKKIMDDEDITEENFIRRLPLLGTPDAGILFHDILSSLLPRLFFDVYQLFAQRSAVEAASSGKTLKKMEEEKKALKAQVSDLNKKLKEKERIIAEKETKIQTLSKKLVTANETSKNIEEVVEKRTEAIAYENKSLMRQNKKLQAYYSDLRSKYDRLKIEMGSSGADVEEDEREEMREVDVNARYLFLLYDDIGCRQNIKQEFPNAEFTSKVVPLSSHKYDMVVSLTECIDHSNYLAMKKQCKIHGVPFAHSPYSNIDMIKTVIWNVLNG

SEQ ID NO: 24 - protein, 1235 aa, synthetic construct
MTAKKKQQPRVWEPPARDALLHAFEQAYAARPDTVTLTIILKLLTQRDTEALLHTAQMRVTNMLHDVTVRFFDDLEQIDAGTTAEHLIDAIIELVSDPIQVDAHHAQAVSQLRASLAVVRTQTIGAMDTTFVSALIAAAASADTADPSHRGWSVLRAFLEQWYIDHPGDKMISTINHAAQATSKRKVKDEHGNLKKDAQGKPMTDVVQTYTPEQVYHIKLALTLRLALVARHEAQVITTRAMALVVLALRQCSNTQAFAWVSTTLTQRFTNTRIHAALALLLVVQQRVHDEACHRIFVTINQRCAGNQMFAVIEVIVELVSRHIPEADNKPAVADTVRHYLYQLTRSRKQRRKPHHVTHANAQVPADETAHADAALFVDEKPHPVRPVLLRIVDALVGSNENTGILPLRMCLEHWYSSFAEQEREERFSAGDRRLIAIARDPTRQDEAGDRHTGMSLLLFHWLQYHCVLSTDVLLLVSLSEKAIRRIGDGDTVLSGILSNCYGEVCSRYQYPLSGELREGVFFNRGGAILGWCQLVADYVAERDKRRPGTTLPPWKTGKPKNVSRVDFFKGNRVVRIGLAYALWKREDTAYKAAVATEAPVGPDEGKTLGNPYAYTELRRQVAGNRRRRVTILGRGSIGKRGARRMKKWMLPAAERRATLRHLDVLLYPELVWNDEERVAAAQASHPYWMKRTKRGITAVRPTQFRLYPSTYPKSLKDLSREKQIQLREELREQERYAPQFAPFRHALDRFLEAAPPAPPFIAPMVLTDADMAQLTPEAQLEWTATFETYKAEFDQNLNWYRDFRPSRGTPTEEYSDEKLYDVETLHNRELEWSAATLAQRHPQPIYFKRGMNAELKPRSFALLYHIPQRKGCARYRFTFVCDLVGETATEREALLTKRRARGEHTVENFLVNHPDQRVTHPNETTLHYFATQMGSDHQERILLEYMDWRRREPERKKPLISTARIVSHKRTRQRTDWYAHLPVPLPAPTHTATLDTVIGFHEHEGTFFYAVVDLDGRLLALGDVDLPNCVRPKRTDGMTNDNFAFETAVRMVWRSMTQQYFHKFRDNPKRYTAFIGIENTGWKQDRVGTKAADNRQNISLPRQRIIEIVTYKAIRHGLPTPLSVKRISPGRDCGHCDARIEHGSGIEKRPVTHCFHCQTLGIEHELEWHKEADNKWSARCITCGRSWRKEEPQFKCQHCRTQQYARYNTALATVHCTVDILVGRKRNTPDSEQE

SEQ ID NO: 25 - protein, 404 aa, synthetic construct
VAKKKPEWLMRTVSVPVRFWPSDVGGDGRLVLDDKGEPVPTHTRQQMWAACGEAWKLSTELANWAGRELLRADVSPPHPGKLPAMPKVYLYGLLTPLLKERWAGQLASANCVLRAVERAYRAKRFEVQARCSAAPAAFRYPYPFRVHNQIWSVHVPADAEMNAGEHPVCTLTLPGIGRVHLPLSRASGHRRALALLRRIAAGDPTVKKGELTVTRQRDGDRNGGHGTDGGPDRAKRAWTIMLRFSVGTRPAENDGTQTMNLVTDPEALLVCWLVGRKDWVVNDDHVRRRIAAHAARRQRLSQDRKAERRLRRGVGIQARLDLECEKHERFMDTYLHQTAAAVAGYAVRNRVGCVFADLSPSGYVDSFPWFKLRERIGNRLAREGVELVDDKWAGSNETSTPEED

SEQ ID NO: 26 - protein, 517 aa, synthetic construct
LEDTSKNVFQSPLLLSKTAETYTPIGYEEAVAITFYYAEDNRRKPGFLRRTGERITLVAPLNYALYIIDIDGRNSVIIDPNKGSELEITYYKPLISLIEEKIEKLRTAETKEFIDILTELKQVLQGMASDKAGVEKYSIRISNILKNRPLIEELKILVRNASENIYEALRIEEKKIDLQGTKNNIREALILLSETRKKLVNLLGEINPIHEKWLEEIRTSYAEKIHEIEQKLEEVKKDVDEKINELKKKQAEEIKNIREKYIGQIETLERRMNEINTKLGQLKTQYEKAKEYGEDTRMIKKRINDLTKKLSDLNKEKKELKEKMNSEMEAVRSKYLEYINAERMRIDKLKKEIEKLNDELESIEKQAGDRLDEIRSLINQLIDKASRAESKLLSIMINTPPHGEGAHYLPVMIITYKSKNKSRRDSFTPAYISVGKLSRTPKISFFEEVRKYLSDIPSILSDARYADQVEKYNLLKTVNPSRIISLLDRLADNGIINRKKVQKIAEKIVEQIKMAQA

SEQ ID NO: 27 - protein, 731 aa, synthetic construct
MAENTNKLPVIGTAYFKDIEGYAAFWKAKDHDWVKTQRKELPPLIYPVDKTHNGVVVKALYRWLYEYWSMSQLTEEELHRRVQDQANEQMKQLREAVYFSETYQQPFQVRALFDFGAYITLGNADFFVFEHEKYLSEITYPKDYSQYTLGELRAIASGVSAGSTVPATLSNEISISQAKDSIQAAKNRLEEMKKEAENLQWERHEELAELKRQAEEAMAKLRQRKSELLDMLEQKREELEDKIEAMNDQVFFLESQIYCIECYLGETVNFAKVRSGRNAPDDEPIVLYQKQRFLDEEMGRLASVYELDFSELNLFEEFLAASPAALEVFAPNERCVTLVRLSKTSLEYGLHPKWANMLKAYDKFHGKMVGIIIRNGENIYIGWTDDDKVIVTDDLFLDFTPAEEVPSDIPVDETERQMKARMRKERAQMRVFAKEAVSRVFIFNILQGIVDNSPILPLPKGEKITKESKYVRFSMADRWLADNRFGSFTDIVSRCNAQFSEGDMLLTTQYLRPENGKHTKNDRGRGYADRTHDVSVQNCAIYPLNLIEYDRPKQVTEYQFKMPACGNTPERWVTSTTDGDGSRLDPKDTRIIRTYTKRDAHYFVSLEKKWSYSGARANFEVFPDEVLNLHYMNSVWLMYAITNRNLGGWRVGGESVDYAFAIRYLKTALDFIRKREADEKMVLDEVAPGFTDRNPEWPVALSEWKLEKKARSFTPVLAKRFVNWAKEREKG

SEQ ID NO: 28 - protein, 491 aa, synthetic construct
MLRRSVAVELEVTKDLNKLFYSVETAYLSIVREVVEYAVANNVTGANKLQRLFYNKYRLEYPSLNSQLIIQAIRQASEIARSFLRRKRKGLVDKPYPEVRRVSIRFSEMAWSYEEFVKSVAPVRLGLSLPNGRMEVWLRPHKRFWQYWWRVLIGEAELASTLMIKRRLNRWYAVFIFELKPRVEKPQSIIAFDINENTVVVGRIDIETTVGKVVDWNRQYVTPQLYMIRTDFGRLARRYEAIRNKIIEKLKPEFALPSGNYVNVANTREFRKRVKRLREGDRKIGRVRQVANELTKTPSIIITEELGENPQESMIEDVGKDELRHRIKQTPFKALVRAVEDKALERGSKLFHVSSYRNSRVCPIHFTRLGETNDWHTLQCPKGHYMHRDYASVTNMAWKLTPESWIKAFWWNMKKDMNWREHEGKSNPLIPHPIVHHLHATLREFMASLEKSPAVLARGNPMNPAGRADEDWARETTRPPGREGGRCRVCG

SEQ ID NO: 29 - protein, 517 aa, synthetic construct
LEETSKKAFQSPLLLSRTAETYTPIGYEEAIAITFYYAEDNRRKPGFLRRTGERITLVAPINYALYIVDIDGRNSIIIDPNKGSELVITYYKPITSLIEEKIYKLRAAESKEFIDILTELKQVLQGMTNDKSGVEKYSIRISNIVKDRTLIEELKILVRNASENIYEALRIEEKKIDLLETKKNIREALILLSDTRKKLVKLLGEINPIHEKWLEEIRTSYAEKIHEIEQKLEEVKKEVDERINELKKKQAEEIKSIREKYIGQIETLERRINEINAKLEQLKAQYEKAKEYGEDTKMIKKRINDLSKKLSDLIKEKKELEEKMNSEIEAVRSKYLEYINAERMRIDKLKKEIEKLNNELDSIEKQAGDRIDEIRALINQLIDKASKAENGIVSIMINTPPHGEGLHYLPVMIITYRSKNKSRRDSFTPAYISTGKLSRTPKISFFEEVRKYLSDIPGILSDARYADQVEKYNLLKTVNPTRIISLLDRLADNGIINRKKVQKIAEKIVEQIKMVQA

SEQ ID NO: 30 - protein, 664 aa, synthetic construct
MIDMVKEGIKEDLVKLYDNISSSSVVEDFYFPIIASNPKFYSTSGLQHYLGTAFLAFRISQHLSLDDYSSATAFLSGLLHDANKWGIPQDEIVKRFENTKLFSIFSEFFGEEKTKKIIKDSMEIAKTLERGGSSRNLQKISEAVRISDYISGDERSWKISRVIDILRNSSLINIGYEHIYPIYLGRQRLVTVYLSHLVKDELIERGMIPLITSSEGMIFLRRSSVSITYDDVIKRMIPLLSSGFRRSKTKSPDASKLVRALEDCKMNLSRSYESLSGYDYDDLLEGLEEAKISRIDHINYLLTLIYVLQRPEGKEHREKREKIFRKISEILNTNLSSSKLCETLITIRDVLMKSDDDFIRRFEEKILEMIREEMREKEVKPDLLRRKISRYISLPREEDVSEDNITRRCPVCGEEILEARPLSAFLKELKKIVGKINTVEIFLPGIQGSPDRPGSIEKLGHKTDVCETCFFEIKELMPRLGLADGLWSAVLTYYPNISIDLADVISNSIKTIVGLDKSSESYKIVPDYMSGRILVGISVRGKGLDKKALSNALKVWLIFGGNMFITTNPLTSPPLIDAPIRLEVGDMIFLECSSKYLEILKKASAYSSYLSFTSQMRYWLFTSLLRYVSSLETTSSGSDAMFRRSFGYTTGFCTLDVLSSESIR

SEQ ID NO: 31 - protein, 1276 aa, synthetic construct
MIYNSLLLNSGGGIIPQFQKSAQSKEAAVLAIGIGGTGVRALSRLKQKVYQQLKSDNEEDDSAVKLYKHIQFLAIDSDETDFKQIRGEAKINADTEFISIQDPALNTIISHNDKKPILDDPRLNWMDIDHITALLAPHGAGGVRQLGRYMIIKKAQRLSTLISGKINLAMENMPNDNVHVYIFAGISGGTGSGCFLDTCYIVRKALKDLGIGGNGKIMGFFFLPDVVTSKREVAANRELVLYNESNGYAAIKELDYLMNLRSAKERFKQVYDEGFVVDTDEPPVDMCHLISAKKADGTVITDGFKYCIHVAADYVMSYLAVVDGNNLEADDTGLTMSGHLANVTAGVGLLPKDAGVNLNYHILGAANAEIPMTQIATYLTTGFIRRFHELVGVKTVTVTKDTVNSFAENIGLSVDDLIEYIHRSNNYPNVQIPDADYQTIYEIGSDNPGVYPRVWVQPRDKAIDSLRGVITYLKNALTSVPDDLDVEKLTANGGESLQVKIFLELCKISAEEEKGPYFASAILGQGGYNLINYIEGQIRQVGEYIQTANKDLYFAQDQAYKAQAEIFKNNGIFANKKKLAKNYKDCVQDWFNYEVEIEELKGAERILETLRISLRNQLKDGFFSKLINMLDNIKDTFKENGDYLNSLRSTQTDAYTIRLVQLTEVRDTLDEAIKKMQPNQLVTDFINRLLKSPKKWLSNDDTQIGQFISTYMQEIFNVEMNKSIQTYLQEKYPDVAGTTELTELVKSTIISELNDKAQPMFWKNPIFNMGASFSTGRMSVPAASNVICNAASSFVENKADKNYTVRKTGINDRVFVLRFHSGLPFYAYQGVTKMYESYRSVANAMPGVGAHLYAKTGRGSGIDDSGMHDWRNELPTPMPYSKVPEFTPNGEELEKLYVKAEEEKVIMPVDINAQPVNYKICYSEKIVKKEFKKEDYLLASGEFNLQKWDEDKKWLENELNRRITRPEKVIDDFADGAADSPVVQRRIHIDYFIHYKYYQKIVLDELESREYLKKAMKRLDDIKKELELYINEVNRYNDIILFGFINCLNATGKFDINRYRIKSINYKYEDKFGVAREEIFAKINDDSEFSSEPLYRGFLHYCKMNPDEEPRRSIDNMLNERKSSTLKAGRDTFIAYQFETNWNKDILADMEIEIKGKLTNEQQEDVKRYYRFMITKVQELKKQFLLNEWQLNPEDENRVEPKKETIRFNENTNTTQEFSNTWECPECGFRKNIGNFCSGCGKAKPKAEVSATWECPECGFRKNIGNFCSGCGRKRD

SEQ ID NO: 32 - protein, 548 aa, synthetic construct
MSDINDIKFKTGWLKDPKDNQKFAPKTTTNQVLTGDGERLTDTLATFITNAADDLKNYYLKSETYTQDEVNELISLIPKFKLEVVASLDDLSATDINEATVYLVSNKSTSGSNVYTEYVYKNNRWEILGEQKLDLSGYVTEEYINNNFEDKNVIKQKLDVVTELVNNNLEAANDYTDEVKEQLSNYETKEAASNKLTEAKQFATNEASKIKNDLLNGAGAAYDTLKELGDLIDESVDAVEALKIIATNKADKEHTHEFTAKYDITTLTELQNASTAGKNIKVVRNFERPIPGAYDENDTMLYSWQELLDMDVEYEEFDGFDETGANVWVTKIDKCLTVEDGALKSIVTYSPSTHSYTNWSIDALTGVTKLVINEGVTAIGPSGLAAFNVAEIIMPSTLTTIEDDALSHIFEYWDWGDYSVTVHIPKAVNNISFNAFSYSKVFIKYEGTETEFNTIASVADFNLNSYEFNYSCNYNLVPAPIYGYIKNKLEDRFIFTAHEDDGEIEYVCNSSGWSTRNIQMMKLGDAVFKYDYDNARIILSFPMEGAVG

SEQ ID NO: 33 - protein, 1138 aa, synthetic construct
MTYRVVGCSECQSLWLLEDTDDESATCPTCGTRHQRHRLRTLAQADDKDGANQQRSALLAERADAGDADLDPYWEQATRASEPIVDDRAYLDGLGIDPNLVEDLTSEDDDTSASEQTTRTPGIGPDVPAPVADRPQLRGPVDDARSPGVSLIEAGGQLGIAGQTYHEVSPRTFEWLADVVSDLVPSTARLVQDLARERYNFDYRDPDAGAGAVATFIHEELVGRVAQLDTADAPTAAVEEARSYLDAIGRYALLWADDVYRVRRGFDGAEQFEHITRTLETTGTSQGQFNAGVDALRHSLVALHAERAVPRTLTFVLDGEQWTGADRETIKRALTAVDALAAGFDVRLWMSPGVRQRVRRLTSTALDDDDAPTWAERFAFLTDAGQTSRRSPEATASSTSADEAEEAWAFVEDNRTQTGLLTLLAQLDADSERSVRALKRDTQIDYADASIDRYVADLEAAGLVAIDRSHASNRLSLTALGIAAQNYVTPTGDVVHRYQSRLVGASYGHPSASHKYSVAPQGTGGDHHPPAERWLADTGDPAEDGYVQFLGDSTGPREIEPPVFHRRMLAGERVSGVNFADADLIDWTNERQAPTGDGRVTYASIFDDHALLITQWGGPAQTMARLCAGLLGNRMLSKALSVDAVGTQFEQTHGGVKAFENDLHDVLVRAQQIGWLSEDELEHYDNWRDRIGAVRSNILSKIGELDELDSGLRRELFRDLQGLLTSATHLYYAAGIDVTINLRLPRTTELLANDEALQHFLDFMRFTVTKQAGYEDENGFHSWYRMCIEDRKEKLKARAAYDVDEADSTADLTASWIISGPGATALKDHVQEAIGEESSRLRERVDEGVEDAATLDIPMVEANSHGHIRGLVREIADRKGFYETNRRDLDRLTRCLEAALATNDRGPDPFLVADALAALESRDTARDRLDTLSVQNALATLPSEAVFPTLPPAARRMLSALFASDEPLDRQALIEVTSESSYDRHWKTLRAFFLVEETDDGFVAQLEPWWASTNEERKPFHEDHPNPVTQPPGGHIKQHAPPTRPYGVIFEIANETDFFEIPPDRYYEFGQRWDEIEWILDELDLERLRPILKAYCADWPDYTAQSYSDPSGVQIAPQQPDSPSTQATLATAATINRQ

SEQ ID NO: 34 - protein, 408 aa, synthetic construct
MELTLLLIIAALLVALLVLTLTRNNRAQSEEMQRALRQQMQENREELNRSIRELRMEMTQTLNQGLQQLQDAMHKNMMTTGELQRQKFDAMARQQETLIQSTEKRLDDMRVMVEEKLQKTLNERIGQSFEIVRSQLENVQKGLGEMKSLAQDVGGLKKVLSNVKMRGTFGEVQLGALLEQMMSPEQYEANVKTKKSGTEFVEFAIKLPGNDDANSTVYLPIDAKFPKDVYEQYYDAFEAGDAALMESCGRQLETTIKKMAKDIHDKYVDPPFTTDFAILFLPFESIYAEVIRRTSLVETLQKDYKIVVTGPTTLGAILNSLQMGFRTLAIQKRTGEVWTVLGAVKTEFGKFGGLLEKVQKNLQSAGDQLEEVMGKRTRAIERKLRQVEELPHEESRRILPIDDGGEDD

SEQ ID NO: 35 - protein, 408 aa, synthetic construct
MELTLLLIIAALLVALLALTLTRNNRAQSEEMQRALRQQMQENREELNRSIRELRMEMTQTLNQGLQQLQDAMHKNMMTTGELQRQKFDAMARQQETLIQSTEKRLDDMRVMVEEKLQKTLNERIGQSFEIVRSQLENVQKGLGEMKSLAQDVGGLKKVLSNVKMRGTFGEVQLGALLEQMMSPEQYEANVKTKKSGTEFVEFAIKLPGKDDANSTVYLPIDAKFPKDVYEQYYDAFEAGDAALMESCGRQLETTIKKMAKDIHDKYVDPPFTTDFAILFLPFESIYAEVIRRTSLVETLQKDYKIVVTGPTTLGAILNSLQMGFRTLAIQKRTGEVWTVLGAVKTEFGKFGGLLEKVQKNLQSAGDQLEEVMGKRTRAIERKLRQVEELPHEESRRILPIDDGGEDD

SEQ ID NO: 36 - protein, 408 aa, synthetic construct
MELTLLLIIAALLVALLVLTLTRNNRAQSEEMQRALRQQMQENREELNRSIRELRMEMTQTLNQGLQQLQDAMHKNMMTTGELQRQKFDAMARQQETLLQSTEKRLDDMRVMVEEKLQKTLNERIGQSFEIVRSQLENVQKGLGEMKSLAQDVGGLKKVLSNVKMRGTFGEVQLGALLEQMMSPEQYEANVKTKKSGTEFVEFAIKLPGKDDANSTVYLPIDAKFPKDVYEQYYDAFEAGDAALMESCGRQLETTIKKMAKDIHDKYVDPPFTTDFAILFLPFESIYAEVIRRTSLVETLQKDYKIVVTGPTTLGAILNSLQMGFRTLAIQKRTGEVWTVLGAVKTEFGKFGGLLEKVQKNLQSAGDQLEEVMGKRTRAIERKLRQVEELPHEESRRILPIDDGGEDD

SEQ ID NO: 37 - protein, 408 aa, synthetic construct
MELTLLLIIAALLVALLVLTLTRNNRAQSEEMQRALRQQMQENREELNRSIRELRMEMTQTLNQGLQQLQDAMHKNMMTTGELQRQKFDAMARQQETLIQSTEKRLDDMRVMVEEKLQKTLNERIGQSFEIVRSQLENVQKGLGEMKSLAQDVGGLKKVLSNVKMRGTFGEVQLGALLEQMMSPEQYEANVKTKKSGTEFVEFAIKLPGNDDANSTVYLPIDAKFPKDVYEQYYDAFEAGDAALMESCGRQLETTIKKMAKDIHDKYVDPPFTTDFAILFLPFESIYAEVIRRTSLVETLQKDYKIVVTGPTTLGAILNSLQMGFRTLAIQKRTGEVWTVLGAVKTEFGNFGGLLEKVQKNLQSAGDQLEEVMGKRTRAIERKLRQVEELPHEESRRILPIDDGGEDD

SEQ ID NO: 38 - protein, 681 aa, synthetic construct
MGSECIYIFRAYRDGRLSDILRDLDRCDSASTVLFTISEYRMVRLRYASEQGYMAGRLLTAYISLKGYAPVTRNILYEWVSRMLAVSSREKAEHNIELLWRDRQIVNLQKNAVEEVIRELSKEGLENLAINPPKPIQKVLETLIKCPRCGSQPSYGEKYCKQCGARIPTKAWETTEETHKPITVEIEQKAGTPTIECPHCGKALRQGAKYCTKCGSSIVSPAHKGMKIREIFRKIQQDVSRKTTPEEPAPELGREVSPESAEKPTLTTFKEPVKSEEKPSQVEEHVKPTAEKPAAEREPRTPTDVGEKPQPQQPAPPTRGVTAPEPVPAGVDWEPDEDMVEAFDDMGVPLEAFRREAIRLALLDVDERVFEREVMRSASKLATSVEGLNRDFRDQLTLSISQYLRDAVFIIRKEIKRPETPRPTVPEHIISEKPAQPALEPPKHVIQQPKPPEQLEKRAPTVESAKQQPPKMDGIELWTLVREGPKPGHVYLLDLPRSALPTSIISKMFEGVVNVEEVDYVGPDTPVALTRTTYRANYISLIKPSPTNPYANKLYRIVEMRCGSCIITTLETYFMNRYVFDSRGLKPFKIILDIEASRKALEKVMHPQYAARLAKLGALLPWLAEELASNGLDGVRNRLHGLLSPEDGEEVYQAIKGVLENDMRYVNKALAISLGIIDEEE

SEQ ID NO: 39 - protein, 444 aa, synthetic construct
MATRIKAIKCPHCGSSNHRQTGEKRFVCNNCGTEFFLDDDDINININHYNHNSDAPLLSMDSDNAKKLKAALLIFFPVLILFFFISSLFHTSPISDKAGKDSIRVREEYLLVQTFAKEDTYIVFCISDRNYYADYNNKSKKQPTNGIYYMFRNVRTGEIVKEGLLISDEQKGDRAFYTFGDNVKLRRFANTRRWYLIIGNRLVYEVNPKRMTLVDAAPQLMSAQKALSSGFAAIGFINKNEAEGFAIQNNLGKIYYYCPATNHLYTKVAFDYATALPANEMPGQCRDSTYYLLQNASSEESENITRIWRVTYKHFNGDPEDRIDRIDDWYLDRLSRYRISEIKPVSSWFISFHPTIIYQDRQHILLQYEATMAPNSPVTLQLRGTDGRVKWTRTLNRELNRTSAEKSSVGYIITTWPYSFIEISNNGKQLTEYTLPEEYQIKAK

SEQ ID NO: 40 - protein, 507 aa, synthetic construct
MSNFCSICGKEFKSSRVGQKACSECLKSEFSVASKMEEEEVEMYRAKQAAAARRQHARMAKLLDSYKNGTMFNMSGKIMFCVGLFILLFCHLIFFLDGRELIYWGVEGMDESGKKAISMLLSFACVLLLVFSTRRFKIVMMLGSVVILLLGWYATAIWKSSSDAMTGGGIVEKKQLPEPGNEAVIPITEEELGVFYELKKNYPRATHYAVFLDNQEPIVRDLVREALTRLLEAEYTRAYTRSNGVLYVVANAAGVPRNVSPIVDRLGEIVNADVAKGLYLVRFDAEKTNLVCKYSSEVLSSPLNSSFVIANINELRCLDAMRIRVAARSLRNANVQMLRREVRDALVRVLGDSWTQDQDTYSALIDSLVTYASAKDREVINICSDYFMKRYGAKKDVLPGVVDYLIKEVPDQMVQPIIDYWLLNPMSWTVQMRNLSWRVQPKLLERMEQEVSIKDLSLILRYLQNNGNEDAIPAVKKLLTHSDAIIRFSAQETLRELESKASEKIEE

SEQ ID NO: 41 - protein, 507 aa, synthetic construct
MSNFCSICGKEFKSSRVGQKACSECLKSEFSAASKMEEEEVEMYRAKQAAAARRQHARMAKLLDSYKNGTMFNMSGKIMFCVGLFILLFCHLIFFLDGRELIYWGVEGMDESGKKAISMLLSFACVLLLVFSTRRFKIVMMLGSVVILLLGWYATAIWKSSSDAMAGGGIVEKKQLPEPGNEAVIPITEEELGVFYELKKNYPRATHYAVFLDNQEPIVRDLVREALTRLLEAEYTRAYTRSNGVLYVVANAAGVPRNVSPIVDRLGEIVNADVAKGLYLVRFDAEKTNLVCKYSSEVLSSPLNSSFVIANINELRCLDAMRIRVAARSLRNANVQMLRREVRDALVRVLGDSWTQDQDTYSALIDSLVTYASAKDREVINICSDYFMKRYGAKKDVLPGVVDYLIKEVPDQMVQPIIDYWLLNPMSWTVQMRNLSWRVQPKLLERMEQEVSIKDLSLILRYLQNNGNEDAIPAVKKLLTHSDAIIRFSAQETLRELESKASEKIEE

SEQ ID NO: 42 - protein, 765 aa, synthetic construct
MALTIIGKARFYNFAGFATWHEALTREQAQKLWQSSICSRQNELGKAYDNVYTNFNAFGFDSGNQERRKRLQKFQDYLELTDQPWDVRYVDHVAKFMVISYGQCDDNTKGKENQFLIENWPEYIEYTPFTDFSAVPMSQLRQITMLGCEGITGGTSLIPQNAGINLSVADVRRGISAQEQALADLQAKVKAVEEGKTEELSPLKKQIDELMAQMNALQEKKKAELSAMKAELEAKKKEMEKQIFILEAEIYSIRCFLGETIQFVKLRSGSNAPLEQPVILYQKLRFMVEDLQKFAIMWPEEKIGKSIEAALKASDSLMDAFAPSTKCITLIRYSETGKVFRAHEEELNILDEYELMHGNQLAILIRNGENLYIGWADEDKITLKDNFFLKPTEAKMEAMPEQRPNESDEYYQARLERMQKDMEEANKKAAREGLSRHFLFSILNGVSASDNSILPLPKFSNKAERNQHIIFSMADGALVDRRFGSFGNIVKRCNSVVKKGDMILTTMSLRAEGPYWSKPGQYTTWYNDRGRGERNRTHDVHASDCTIYPVNLVEFDKPERKVRYRSTYTIPEEVNGEYRQVKRTSIHVTDADAKLSEDVEILEYFDYIQQHTFISLQKEGGWWKCDVGPSYANFEVNTDEFINLTYMNSVWLNYVLSCRDLSGWRVGGTTVNYEYALRYLKKALSHIREREKTEEENIRLAGGAAILADPDWPVALSEWKMDKGVRNMTEYQAKRFVKAMLAGEIDITPKPVAEGDVEMYDAVIV

SEQ ID NO: 43 - protein, 516 aa, synthetic construct
MVDAAARSPGRLLVFVHIPKTAGTTLRTVLGMNEPGARSRALGNVFKGGGGISKSLIDRLRKDKGPDLSGVGLIRGHFPLGIREHLPRYLPRGREVACFTFLREPADRTLSHYFAIREHGRGYQLPPLPAEATVEDALERGYIHDNVQTRMLSGLAEPFGEVDQEMLERAKDNLREELVFFGLTERFDESLVLAKRRLGFRSILYKTSGRVNVNRARGDEVPAELREAAQACNRYDIELYRYAEQLFETAPERQELEFDAELAALRAAKANGDGESTSISVPASYGGDEQAWTMLVEARAELLRLEFERRRHRVPHVAATVQDEVRESELKAARTRTKKLEQEVERLKTAQAKTKRLEQQVERLKAASSRVEELEREVERLNAVASRAEELEQRLEQQVEHLNAASAKTEKFEQQVERLKAARSRNRKLQQEVERLKTAVARTEELEEEVQRLETAAAKAEGLEEEVERLKTAASRAEELEHELEQQLKLLDAARSRKTKLEQKVERLRDAAAVRE

SEQ ID NO: 44 - protein, 1123 aa, synthetic construct
MDKTALEPTRTYHATVDLGENTEAFLAHHVATNNAAGELAGALKHMIATLVPAQWVELWENHAGTPAGVRIASWIAGSWVGTERSDSAPPRTVTHAQAIDAVRRRAVELRPGIDAEQFLKDAAILHGTVRDDAVWVDYLDGDPVPAIGWEGLDVRLLGSASEPVKSNDEITKPGKGDMVQSCSRLWGRAVGTGRKADGLLLVERSAALRDAAADPTNRTVADVAQALTGQPLDGAGLAAWANHTNPKSPTKVTAALVGNDASADLSTEVAGKTLRDRIVTGASEVHANWVARTGEARRAADGEILAGTWLHGILLRHMAANLTGGARTNVLSAAASTVATHRDRLHKQAVELAALQQSLANALEQVDPDDLADLDAWARAHLYENEDSPGYLLPRIPRREGTAKLATVTNKWRNKPKMGVEERVAAAKGAGAGWTVWWDYLARNEHLWNPNTDRLLRVVGGGDGFAGVQPLRRKIAGVQMPTFARPHPQAAPAPSYYGGGRLTGRLGADGTLVLQVATPDGLARISARWMSTRLREALKVGAQVVDGQPTITRDDPTSRLAAGVDQDTPVNVAADPTSSTFTVGYDRENQRWLLAVAIKHHTPRRYLPGDPLRLMGVQIGMNRHDVTYAVWETVDGVSAPQDVEVADVHAVNARGQATVRRARRVAATGNLFEFARPVREGSIDLPAGDRESQVGESAQIAELLDDLPVHDAYIQGELRRVARDAAVDLGAGTPRSQLLQAAMRVLNRALDAQGCLARLAGQARGLGEAGPDDLEELESIWPATGGDLGALWSQRAATIARYARIVRDDLLTRAHSRRVSVERVVELDEFARLLRRLETCPTPSRPFEHRTLPFGFAPTIRTMRNRAREAWTDKIASDTVKTALRLGVSVIAVYKDEDSTPSQVRPAHINRRLATEARRAIADQIALVAPLHGIEVKVVSKGVGHRARVVDGREMFAVTPITLGEFADLARRDTKGAKGAIASWDNRVVAAANLLAGKEKKRKGRDGRVYEESAQRKAANLAVAALTVQAGQFLDRELEPVLGLNDRAFRTWRSSAAASQTLHLPGLFPDGQVLVPADAQYEPGTLRFRSVSSRDIAIAHAVAHRGCTWVLNPKKQKKDDESE

SEQ ID NO: 45 - protein, 1073 aa, synthetic construct
VRPAVRNYNVAAGAHKQERDERTYIKGAFGSFSREEDNRASTQFLRPAGSQHYSAILASFTDENRDDTFTLAVLLYLNSEGGADKVYCFANGERSIAADCSRIETVEKLRQQMEWRGFHATTTYTEYYGWFRRKTGVQFKAMDVFNQTVAVKDIKSLNQFVREHMLEAKPWSEKVDSLLNHFTQLHEAHQSLLRVQRQFQLLEPVGEAGEVYRVHAEKLESVRRLIDATDSFFRQKTVDILGPSAKEWKDLRHAVGRQRERLDQDIREVLEECRRLQNEIEQAGGDRLRQIPLLIKQHESNAATKRTANGHYHGALAAASIRETVADAEGFARIQAQIPQLVRDLDQQIEETDKRKEDLVIERATLNQSIRNDETELESLKQRQGNLPHALVTVRQTICTDLQLPERELPFVAELVAVKPEEQAWEASAELALRTFALSLLVPHRHYPLVAGFVDRTRITDAAGAGQRLVYLRVGDRAKVAAQAALHPQSLIRKLTFRDGHPLLPWVKGELEDRFDYRCCETLEEFQEAHGLAMTRHRHLKIRGVRHEKDDRDRVADPRFYVLGWNNSDKKRRLVERIREHQESRTQLNKRIEGIDAKLAALRTRHEALRKAGEVADFGEIDYRVHEWELEALRLEKKQLEENNDAIRLLQGRLADAESRHTGLRASRDDALLKEAKLTDEISQAEKLVANAETVLHERQVSGALQHHASSFPDLDACFIEQTLTAATLLAEERRFIDARRAECDRLGREIEPKKLDLTRAMSRYLREFQDDRADLEAQAEYLDSFLDLRDHIRKEDLPRHEKRFKAHLNEKVIQEIGILNGTFQSERSEIITRINQLNASLRQLEYRPGTHMVLEPRAVRDGDIVDFQNALRECLAGTFEGSPEADEARFIRIEKPITRLREEQTWREKVTDVRRWFDFAARELENVTNKERSYHEDSAGQSGGEKAKLAFTILVAAIAYQYDIDPDHPTSSRFHFVVVDEMFSKVDDQYSEYALELFKKFGLQLLIVAPLDAKALVTEPYVGCYLHAVKDPRTNRSEVFTMSAREFADTLSAAGQGQKNAMGPAPVKVRSR

SEQ ID NO: 46 - protein, 1118 aa, synthetic construct
MKTDDEKKDTAIKSYTSWLLPNDRKEKDMVRSFLALDQGSRFFFDLMQAWYGGLTPDIIKSVTKERDLVDLWCAIYWFRPMKKELASHPIDRVDLAKTFQRYYGGPASDVVQEYLSASIGEDCCWNDCRQKYQEFCKNLGVDFTPDLKTLVREKLIAVALNDGSQAAISNLFGNGGKEDRNVKVNICKKILIALEKAGKSVKYVRDVQSIILKCANAKDREDFNRIYADKNGRPGTLLLLLDRKGENTYDAEVLKRYLRKTIKSKNAPLVWNYNQKLFEYIERGIRNLLRRTINYDAWAWGEMWRVGHTPLQTKATRNYHFAQEQLKRHSDIAAASQSSHAIAVALNEFFESPFFDGENPFTICPHHLGKNLEKLFKAWADEKNQTDEEDAGETAITDYCAEFKDGFDREPIRNLLRYVYASLRSKYSAKELIQAAKYNQQFERYQRQKAHPVTPDNQGYTWPEAVIKPSKAERKNRENSLDTRIWVSVKLLQKDGTWEKHHIPFYNLRFFEEVYAAASIAEDMPSPTFRNSRFGFKLPKLLNTHAKINKKKKDGKKDKKAVNAAKREARIYLAAKEGRLPAPSVPLDKLSAVIARVNGKFQVTVPVKFKVNKQPKYPLPQLGQIILGYDQNLTANHAWALFEVVTEDTPGAHFCRNQYLRVLKTGQVRSITKTKDGQEIDQLSYDGLEYKEYGEWRKQAKKFASQWYITTKVRKGKETTLLSQPALERFESIEKYKPALYRFNKEYARLLLAVIRRKTLNELEEIRPEIFRLVEQGFGICRFGSLSLSSFEAIRAAKAVIYGYFSTALKHDHNTPITDDERQAFDPELFGLLNKLETLRQNKGEEKINRAANALIRIALENKADFIRGEGDLPTTNKSVKKGQNSRSMDWLARGLFNKICQLAVMHNIIPTAANPHSTSHQDPFEHNKDFDDPQPAMKCRYAEFKIEDIGDSVLERLGTSLRNAKAGQTGAYYYKGAQDFLAHYGLQDIEEELKKCRRGRKANMPCWELQKRLIQKLGSKDAVVYIPMRGGRIYLATHSVTTGAKPIIFNGEEVWVSNADEIAAVNIGLTIIPTSKKDEEHPDRENDDKAIRKSVRRPRKSGERRSGDKMPATARKT

SEQ ID NO: 47 - protein, 640 aa, synthetic construct
MDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 48 - protein, 1018 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 49 - protein, 1035 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQTNEKAKDILNKINDNLIGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 50 - protein, 1037 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQKEDILSAANILNDFRRGEFGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 51 - protein, 1041 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQKDILLAASILGDFRRGEFNRSVVGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 52 - protein, 1021 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQNILGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 53 - protein, 933 aa, synthetic construct
MSIVAEPESVADAAEAAPKKKKSDYFTTTRSASLKSTVNPAFEMYEAFQRDFRKNIEILFPWLGIGYGVPDADAKGGIRYVGPEEAVAARNASLPSAKAVAIVERFVTCLSKNAKTLSSCYSLYYKHYKPTGAKRGESVKKIEKLLESDSFSDLAKSHPGIDWQAAKLVPKDKLWGHLKPQLTDKNREIGREVRQFVTLPCSYPEACDYCKLLWSAVLAFLQSRLEIYQYHMDGTAALKTAFDKGNTPLYAAFVQYTDAVAAMLGNSLSPREILHVIECAKGRKIAERPHLQLAIDELKKPAYALLLAAETATVKRCFATRTVAAKLKKRKAYPTKSALDKDQTMNVGSPMFSIPDTSPVSPTKHITVTVTVNGEQVEFPCKHTEYLANLTLEKVSPKKGEAYYKMGFNTNHKARTLGELKTVSIQKRGGNFYVTLPYTITHPVANFNLAWRFFQAASPKMENLVLPDQVVCASYDLGINNPVTGCLFKIGRDLQGPIKARGYGEGYILKLPTYLTSSGSQKNGSAYCQAVVALRNKLGLFHDAMKGLKIHRNTGFPMLEDEVEFLRSAPNMSEKSRQHVAKKNDLRLCVSYWMGFLNAERKRLNYELRATGYKDQVGELIRNIELDDIYYKVRSRYDSLDVSRETPRRYDTSRANRKVNANRLIASAIARDCLANGVWIVFVENLASKYAKGSANLLAKLFNASTLLDTIKAALDKIGVALVRVEKNGTSQHDPVTGALGYRDDTNHENLIVSRDGELCSIPCDKSASLCVGLVGVNHSVVPHKFYVPKPDAEAEAEEEEEEDGGSKRLTAFLADAGKTLKDLKNLTAGPGGVVRFTSDPTAKRIAGQSVYPCHATGKLYTAAAKDAIQDGIRNRCKAGEVPRPAEVIPDGAKTYKAFSTGLVLELAQPSGDQELENIVLPKKRSRSRRVRD

SEQ ID NO: 54 - RNA, 36 nt, synthetic construct
  source 1..36: mol_type = other RNA
gtgcttttactcatttctagcgaactgaataagcac

SEQ ID NO: 55 - RNA, 36 nt, synthetic construct
  source 1..36: mol_type = other RNA
gtgcttattcagttcgctagaaatgagtaaaagcac

SEQ ID NO: 56 - RNA, 36 nt, synthetic construct
  source 1..36: mol_type = other RNA
gtgcttttactcatttctaacgaattgaataagcac

SEQ ID NO: 57 - RNA, 36 nt, synthetic construct
  source 1..36: mol_type = other RNA
gtgcttattcaattcgttagaaatgagtaaaagcac

SEQ ID NO: 58 - RNA, 36 nt, synthetic construct
  source 1..36: mol_type = other RNA
gtgcttttactcatttctagcgaattgaataagcac

SEQ ID NO: 59 - RNA, 36 nt, synthetic construct
  source 1..36: mol_type = other RNA
gtgcttattcaattcgctagaaatgagtaaaagcac

SEQ ID NO: 60 - RNA, 36 nt, synthetic construct
  source 1..36: mol_type = other RNA
gtgcttttacccgtttctagcgaattggataagcac

SEQ ID NO: 61 - RNA, 36 nt, synthetic construct
  source 1..36: mol_type = other RNA
gtgcttatccaattcgctagaaacgggtaaaagcac

SEQ ID NO: 62 - RNA, 36 nt, synthetic construct
  source 1..36: mol_type = other RNA
gtgcttttacttatttctaacgaattaaataagcac

SEQ ID NO: 63 - RNA, 36 nt, synthetic construct
  source 1..36: mol_type = other RNA
gtgcttatttaattcgttagaaataagtaaaagcac

SEQ ID NO: 64 - RNA, 36 nt, synthetic construct
  source 1..36: mol_type = other RNA
gtgtttttacccatttctagcgaattggataagcac

SEQ ID NO: 65 - RNA, 36 nt, synthetic construct
  source 1..36: mol_type = other RNA
gtgcttatccaattcgctagaaatgggtaaaaacac

SEQ ID NO: 66 - RNA, 36 nt, synthetic construct
  source 1..36: mol_type = other RNA
gtgcttttactcatttctaacgaattgaacaagcac

SEQ ID NO: 67 - RNA, 36 nt, synthetic construct
  source 1..36: mol_type = other RNA
gtgcttgttcaattcgttagaaatgagtaaaagcac

SEQ ID NO: 68 - RNA, 36 nt, synthetic construct
  source 1..36: mol_type = other RNA
gtgcttttactcatttctaacgaactgaataagcac

SEQ ID NO: 69 - RNA, 36 nt, synthetic construct
  source 1..36: mol_type = other RNA
gtgcttattcagttcgttagaaatgagtaaaagcac

SEQ ID NO: 70 - RNA, 36 nt, synthetic construct
  source 1..36: mol_type = other RNA
gtgtttttattcatttctagcgaactgaataagcac

SEQ ID NO: 71 - RNA, 36 nt, synthetic construct
  source 1..36: mol_type = other RNA
gtgcttattcagttcgctagaaatgaataaaaacac

SEQ ID NO: 72 - RNA, 36 nt, synthetic construct
  source 1..36: mol_type = other RNA
gtgcttttactcacttctaacgaattaaataagcac

SEQ ID NO: 73 - RNA, 36 nt, synthetic construct
  source 1..36: mol_type = other RNA
gtgcttatttaattcgttagaagtgagtaaaagcac

SEQ ID NO: 74 - RNA, 36 nt, synthetic construct
  source 1..36: mol_type = other RNA
gtgcttttactcatttctaacgaatcgaataagcac

SEQ ID NO: 75 - RNA, 36 nt, synthetic construct
  source 1..36: mol_type = other RNA
gtgcttattcgattcgttagaaatgagtaaaagcac

SEQ ID NO: 76 - RNA, 36 nt, synthetic construct
  source 1..36: mol_type = other RNA
gtgcttttatccatttctaacgaattgaataagcac

SEQ ID NO: 77 - RNA, 36 nt, synthetic construct
  source 1..36: mol_type = other RNA
gtgcttattcaattcgttagaaatggataaaagcac

SEQ ID NO: 78 - RNA, 36 nt, synthetic construct
  source 1..36: mol_type = other RNA
gttacaatacctagaggttttaccactagattgtac

SEQ ID NO: 79 - RNA, 36 nt, synthetic construct
  source 1..36: mol_type = other RNA
gtacaatctagtggtaaaacctctaggtattgtaac

SEQ ID NO: 80 - RNA, 36 nt, synthetic construct
  source 1..36: mol_type = other RNA
gttacaatacctagaggtctttccaataggttgtac

SEQ ID NO: 81 - RNA, 36 nt, synthetic construct
  source 1..36: mol_type = other RNA
gtacaacctattggaaagacctctaggtattgtaac

SEQ ID NO: 82 - RNA, 31 nt, synthetic construct
  source 1..31: mol_type = other RNA
gttgcgaacacatggcatctagcgttgccat

SEQ ID NO: 83 - RNA, 31 nt, synthetic construct
  source 1..31: mol_type = other RNA
atggcaacgctagatgccatgtgttcgcaac

SEQ ID NO: 84 - RNA, 31 nt, synthetic construct
  source 1..31: mol_type = other RNA
gttgcgaacacatggcatctagcgttgccac

SEQ ID NO: 85 - RNA, 31 nt, synthetic construct
  source 1..31: mol_type = other RNA
gtggcaacgctagatgccatgtgttcgcaac

SEQ ID NO: 86 - RNA, 31 nt, synthetic construct
  source 1..31: mol_type = other RNA
gttgcgaacagatggcatctagcgttgccat

SEQ ID NO: 87 - RNA, 31 nt, synthetic construct
  source 1..31: mol_type = other RNA
atggcaacgctagatgccatctgttcgcaac

SEQ ID NO: 88 - RNA, 40 nt, synthetic construct
  source 1..40: mol_type = other RNA
tgttgcgaacacatggcatctagcgttgccatgcagcaca

SEQ ID NO: 89 - RNA, 40 nt, synthetic construct
  source 1..40: mol_type = other RNA
tgtgctgcatggcaacgctagatgccatgtgttcgcaaca

SEQ ID NO: 90 - RNA, 36 nt, synthetic construct
  source 1..36: mol_type = other RNA
gtgctggcgacctgctacggcggatcgccaaggcac

SEQ ID NO: 91 - RNA, 36 nt, synthetic construct
  source 1..36: mol_type = other RNA
gtgccttggcgatccgccgtagcaggtcgccagcac

SEQ ID NO: 92 - RNA, 36 nt, synthetic construct
  source 1..36: mol_type = other RNA
gtgctggcgacctgctacggtggatcgccaaggcac

SEQ ID NO: 93 - RNA, 36 nt, synthetic construct
  source 1..36: mol_type = other RNA
gtgccttggcgatccaccgtagcaggtcgccagcac

SEQ ID NO: 94 - RNA, 37 nt, synthetic construct
  source 1..37: mol_type = other RNA
agtgctggttctcgtcgctaggggagagtcaaagcac

SEQ ID NO: 95 - RNA, 37 nt, synthetic construct
  source 1..37: mol_type = other RNA
gtgctttgactctcccctagcgacgagaaccagcact

SEQ ID NO: 96 - RNA, 37 nt, synthetic construct
  source 1..37: mol_type = other RNA
ggtacaatctagtggtaaaacctctaggtattgtaac

SEQ ID NO: 97 - RNA, 37 nt, synthetic construct
  source 1..37: mol_type = other RNA
gttacaatacctagaggttttaccactagattgtacc

SEQ ID NO: 98 - RNA, 37 nt, synthetic construct
  source 1..37: mol_type = other RNA
agtacaacctattggaaagacctctaggtattgtaac

SEQ ID NO: 99 - RNA, 37 nt, synthetic construct
  source 1..37: mol_type = other RNA
gttacaatacctagaggtctttccaataggttgtact

SEQ ID NO: 100 - RNA, 37 nt, synthetic construct
  source 1..37: mol_type = other RNA
tgtacaatctagtggtaaaacctctaggtattgtaac

SEQ ID NO: 101 - RNA, 37 nt, synthetic construct
  source 1..37: mol_type = other RNA
gttacaatacctagaggttttaccactagattgtaca

SEQ ID NO: 102 - RNA, 36 nt, synthetic construct
  source 1..36: mol_type = other RNA
gctgcatggcaacgctagatgccatgtgttcgcaac

SEQ ID NO: 103 - RNA, 36 nt, synthetic construct
  source 1..36: mol_type = other RNA
gttgcgaacacatggcatctagcgttgccatgcagc

SEQ ID NO: 104 - RNA, 36 nt, synthetic construct
  source 1..36: mol_type = other RNA
gctgcgtggcaacgctagatgccatgtgttcgcaac

SEQ ID NO: 105 - RNA, 36 nt, synthetic construct
  source 1..36: mol_type = other RNA
gttgcgaacacatggcatctagcgttgccacgcagc

SEQ ID NO: 106 - RNA, 36 nt, synthetic construct
  source 1..36: mol_type = other RNA
gctgtatggcaacgctagatgccatctgttcgcaac

SEQ ID NO: 107 - RNA, 36 nt, synthetic construct
  source 1..36: mol_type = other RNA
gttgcgaacagatggcatctagcgttgccatacagc

SEQ ID NO: 108 - RNA, 30 nt, synthetic construct
  source 1..30: mol_type = other RNA
tggcaacgctagatgccatgtgttcgcaac

SEQ ID NO: 109 - RNA, 30 nt, synthetic construct
  source 1..30: mol_type = other RNA
gttgcgaacacatggcatctagcgttgcca

SEQ ID NO: 110 - RNA, 30 nt, synthetic construct
  source 1..30: mol_type = other RNA
tggcaacgctagatgccatctgttcgcaac

SEQ ID NO: 111 - RNA, 30 nt, synthetic construct
  source 1..30: mol_type = other RNA
gttgcgaacagatggcatctagcgttgcca

SEQ ID NO: 112 - RNA, 36 nt, synthetic construct
  source 1..36: mol_type = other RNA
gtgctgttccttcccgtcagagaaggaagagggcac

SEQ ID NO: 113 - RNA, 36 nt, synthetic construct
  source 1..36: mol_type = other RNA
gtgccctcttccttctctgacgggaaggaacagcac

SEQ ID NO: 114 - RNA, 33 nt, synthetic construct
  source 1..33: mol_type = other RNA
ctgttccttcccgtcagagaaggaagagggcac

SEQ ID NO: 115 - RNA, 33 nt, synthetic construct
  source 1..33: mol_type = other RNA
gtgccctcttccttctctgacgggaaggaacag

SEQ ID NO: 116 - RNA, 36 nt, synthetic construct
  source 1..36: mol_type = other RNA
atagcaatttcatctaatgccgttaggcgttgagca

SEQ ID NO: 117 - RNA, 36 nt, synthetic construct
  source 1..36: mol_type = other RNA
tgctcaacgcctaacggcattagatgaaattgctat

SEQ ID NO: 118 - RNA, 29 nt, synthetic construct
  source 1..29: mol_type = other RNA
ccccaagcccgcatgcgcgtgggctggac

SEQ ID NO: 119 - RNA, 29 nt, synthetic construct
  source 1..29: mol_type = other RNA
gtccagcccacgcgcatgcgggcttgggg

SEQ ID NO: 120 - RNA, 29 nt, synthetic construct
  source 1..29: mol_type = other RNA
taaaaggcccacattcgcgtgggctggac

SEQ ID NO: 121 - RNA, 29 nt, synthetic construct
  source 1..29: mol_type = other RNA
gtccagcccacgcgaatgtgggcctttta

SEQ ID NO: 122 - RNA, 29 nt, synthetic construct
  source 1..29: mol_type = other RNA
ccccaagccctcgcgcacgtgggccggac

SEQ ID NO: 123 - RNA, 29 nt, synthetic construct
  source 1..29: mol_type = other RNA
gtccggcccacgtgcgcgagggcttgggg

SEQ ID NO: 124 - RNA, 29 nt, synthetic construct
  source 1..29: mol_type = other RNA
ccccaaaccctcgcgcacgtgggctggac

SEQ ID NO: 125 - RNA, 29 nt, synthetic construct
  source 1..29: mol_type = other RNA
gtccagcccacgtgcgcgagggtttgggg

SEQ ID NO: 126 - RNA, 30 nt, synthetic construct
  source 1..30: mol_type = other RNA
ccccaagccctcgcgcacgtgggccggact

SEQ ID NO: 127 - RNA, 30 nt, synthetic construct
  source 1..30: mol_type = other RNA
agtccggcccacgtgcgcgagggcttgggg

SEQ ID NO: 128 - RNA, 23 nt, synthetic construct
  source 1..23: mol_type = other RNA
ccgaccacccccgcccgcgtggg

SEQ ID NO: 129 - RNA, 23 nt, synthetic construct
  source 1..23: mol_type = other RNA
cccacgcgggcgggggtggtcgg

SEQ ID NO: 130 - RNA, 23 nt, synthetic construct
  source 1..23: mol_type = other RNA
acgaccacccccgcccgcgtggg

SEQ ID NO: 131 - RNA, 23 nt, synthetic construct
  source 1..23: mol_type = other RNA
cccacgcgggcgggggtggtcgt

SEQ ID NO: 132 - RNA, 23 nt, synthetic construct
  source 1..23: mol_type = other RNA
gcaaacacactagcccgcgtggg

SEQ ID NO: 133 - RNA, 23 nt, synthetic construct
  source 1..23: mol_type = other RNA
cccacgcgggctagtgtgtttgc

SEQ ID NO: 134 - RNA, 23 nt, synthetic construct
  source 1..23: mol_type = other RNA
gcgaccacccccgcccgcgtggg

SEQ ID NO: 135 - RNA, 23 nt, synthetic construct
  source 1..23: mol_type = other RNA
cccacgcgggcgggggtggtcgc

SEQ ID NO: 136 - RNA, 29 nt, synthetic construct
  source 1..29: mol_type = other RNA
ccgaccacccccgcccgcgtgggcttgac

SEQ ID NO: 137 - RNA, 29 nt, synthetic construct
  source 1..29: mol_type = other RNA
gtcaagcccacgcgggcgggggtggtcgg

SEQ ID NO: 138 - RNA, 23 nt, synthetic construct
  source 1..23: mol_type = other RNA
caaccaaccaccgaccgcgtggg

SEQ ID NO: 139 - RNA, 23 nt, synthetic construct
  source 1..23: mol_type = other RNA
cccacgcggtcggtggttggttg

SEQ ID NO: 140 - RNA, 23 nt, synthetic construct
  source 1..23: mol_type = other RNA
ccgaccacccccgccagcgtggg

SEQ ID NO: 141 - RNA, 23 nt, synthetic construct
  source 1..23: mol_type = other RNA
cccacgctggcgggggtggtcgg

SEQ ID NO: 142 - RNA, 37 nt, synthetic construct
  source 1..37: mol_type = other RNA
gttggattcagccataaaatccagtaggtataaaacc

SEQ ID NO: 143 - RNA, 37 nt, synthetic construct
  source 1..37: mol_type = other RNA
ggttttatacctactggattttatggctgaatccaac

SEQ ID NO: 144 - RNA, 37 nt, synthetic construct
  source 1..37: mol_type = other RNA
gttggattcagccataaaatccagtaggtataaaact

SEQ ID NO: 145 - RNA, 37 nt, synthetic construct
  source 1..37: mol_type = other RNA
agttttatacctactggattttatggctgaatccaac

SEQ ID NO: 146 - RNA, 32 nt, synthetic construct
  source 1..32: mol_type = other RNA
gattcagccataaaatccagtaggtataaaac

SEQ ID NO: 147 - RNA, 32 nt, synthetic construct
  source 1..32: mol_type = other RNA
gttttatacctactggattttatggctgaatc

SEQ ID NO: 148 - RNA, 36 nt, synthetic construct
  source 1..36: mol_type = other RNA
gttggattcagccataaaatccagtaggtataaaac

SEQ ID NO: 149 - RNA, 36 nt, synthetic construct
  source 1..36: mol_type = other RNA
gttttatacctactggattttatggctgaatccaac

SEQ ID NO: 150 - RNA, 32 nt, synthetic construct
  source 1..32: mol_type = other RNA
gattcagccttcaaatccagttgttttctatt

SEQ ID NO: 151 - RNA, 32 nt, synthetic construct
  source 1..32: mol_type = other RNA
aatagaaaacaactggatttgaaggctgaatc

SEQ ID NO: 152 - RNA, 32 nt, synthetic construct
  source 1..32: mol_type = other RNA
gattcagcctacaaatccagtaggtgtaaaac

SEQ ID NO: 153 - RNA, 32 nt, synthetic construct
  source 1..32: mol_type = other RNA
gttttacacctactggatttgtaggctgaatc

SEQ ID NO: 154 - RNA, 32 nt, synthetic construct
  source 1..32: mol_type = other RNA
gattcagtcataaaatccagtaggtataaaac

SEQ ID NO: 155 - RNA, 32 nt, synthetic construct
  source 1..32: mol_type = other RNA
gttttatacctactggattttatgactgaatc

SEQ ID NO: 156 - RNA, 38 nt, synthetic construct
  source 1..38: mol_type = other RNA
gttggattcagccataaaatccagtaggtataaaaccg

SEQ ID NO: 157 - RNA, 38 nt, synthetic construct
  source 1..38: mol_type = other RNA
cggttttatacctactggattttatggctgaatccaac

SEQ ID NO: 158 - RNA, 23 nt, synthetic construct
  source 1..23: mol_type = other RNA
gttggattcagccataaaatcca

SEQ ID NO: 159 - RNA, 23 nt, synthetic construct
  source 1..23: mol_type = other RNA
tggattttatggctgaatccaac

SEQ ID NO: 160 - RNA, 23 nt, synthetic construct
  source 1..23: mol_type = other RNA
gttggattcagcctacaaatcca

SEQ ID NO: 161 - RNA, 23 nt, synthetic construct
  source 1..23: mol_type = other RNA
tggatttgtaggctgaatccaac

SEQ ID NO: 162 - RNA, 23 nt, synthetic construct
  source 1..23: mol_type = other RNA
gttggattcagtcataaaatcca

SEQ ID NO: 163 - RNA, 23 nt, synthetic construct
  source 1..23: mol_type = other RNA
tggattttatgactgaatccaac

SEQ ID NO: 164 - RNA, 23 nt, synthetic construct
  source 1..23: mol_type = other RNA
tggatttgaaggttgaatccaac

SEQ ID NO: 165 - RNA, 23 nt, synthetic construct
  source 1..23: mol_type = other RNA
gttggattcaaccttcaaatcca

SEQ ID NO: 166 - RNA, 23 nt, synthetic construct
  source 1..23: mol_type = other RNA
tgggtttgagggctgaatccaag

SEQ ID NO: 167 - RNA, 23 nt, synthetic construct
  source 1..23: mol_type = other RNA
cttggattcagccctcaaaccca

SEQ ID NO: 168 - RNA, 23 nt, synthetic construct
  source 1..23: mol_type = other RNA
tggatttgtagactgaatccaac

SEQ ID NO: 169 - RNA, 23 nt, synthetic construct
  source 1..23: mol_type = other RNA
gttggattcagtctacaaatcca

SEQ ID NO: 170 - RNA, 23 nt, synthetic construct
  source 1..23: mol_type = other RNA
tgactttgagggctgaagccaac

SEQ ID NO: 171 - RNA, 23 nt, synthetic construct
  source 1..23: mol_type = other RNA
gttggcttcagccctcaaagtca

SEQ ID NO: 172 - RNA, 42 nt, synthetic construct
  source 1..42: mol_type = other RNA
ggttttatacctactggattttatggctgaatccaaccgagc

SEQ ID NO: 173 - RNA, 42 nt, synthetic construct
  source 1..42: mol_type = other RNA
gctcggttggattcagccataaaatccagtaggtataaaacc

SEQ ID NO: 174 - RNA, 36 nt, synthetic construct
  source 1..36: mol_type = other RNA
cttccagagcctcccgatcggcttcgtagatcagac

SEQ ID NO: 175 - RNA, 36 nt, synthetic construct
  source 1..36: mol_type = other RNA
gtctgatctacgaagccgatcgggaggctctggaag

SEQ ID NO: 176 - RNA, 36 nt, synthetic construct
  source 1..36: mol_type = other RNA
tctccagagcctcccgatcggcttcgtagatcagac

SEQ ID NO: 177 - RNA, 36 nt, synthetic construct
  source 1..36: mol_type = other RNA
gtctgatctacgaagccgatcgggaggctctggaga

SEQ ID NO: 178 - RNA, 36 nt, synthetic construct
  source 1..36: mol_type = other RNA
cttccagagccttccgatcggcttcgtagatcagac

SEQ ID NO: 179 - RNA, 36 nt, synthetic construct
  source 1..36: mol_type = other RNA
gtctgatctacgaagccgatcggaaggctctggaag

SEQ ID NO: 180 - RNA, 36 nt, synthetic construct
  source 1..36: mol_type = other RNA
cttccagagcctcccgatcggcttcataggtcggac

SEQ ID NO: 181 - RNA, 36 nt, synthetic construct
  source 1..36: mol_type = other RNA
gtccgacctatgaagccgatcgggaggctctggaag

SEQ ID NO: 182 - RNA, 36 nt, synthetic construct
  source 1..36: mol_type = other RNA
cttccagagccccctgatcggcttcgtagatcagac

SEQ ID NO: 183 - RNA, 36 nt, synthetic construct
  source 1..36: mol_type = other RNA
gtctgatctacgaagccgatcagggggctctggaag

SEQ ID NO: 184 - RNA, 36 nt, synthetic construct
  source 1..36: mol_type = other RNA
cttccaaagcctcccgatcggcttcgtagatcagac

SEQ ID NO: 185 - RNA, 36 nt, synthetic construct
  source 1..36: mol_type = other RNA
gtctgatctacgaagccgatcgggaggctttggaag

SEQ ID NO: 186 - RNA, 36 nt, synthetic construct
  source 1..36: mol_type = other RNA
cttccagagcctcctgttcggcttcgtagatcagaa

SEQ ID NO: 187 - RNA, 36 nt, synthetic construct
  source 1..36: mol_type = other RNA
ttctgatctacgaagccgaacaggaggctctggaag

SEQ ID NO: 188 - RNA, 36 nt, synthetic construct
  source 1..36: mol_type = other RNA
cttccagagcctcctgatcggcttcgtaggtcagac

SEQ ID NO: 189 - RNA, 36 nt, synthetic construct
  source 1..36: mol_type = other RNA
gtctgacctacgaagccgatcaggaggctctggaag

SEQ ID NO: 190 - RNA, 36 nt, synthetic construct
  source 1..36: mol_type = other RNA
cttccagagccttctgatcggttcggtgctgggact

SEQ ID NO: 191 - RNA, 36 nt, synthetic construct
  source 1..36: mol_type = other RNA
agtcccagcaccgaaccgatcagaaggctctggaag

SEQ ID NO: 192 - RNA, 34 nt, synthetic construct
  source 1..34: mol_type = other RNA
tccagagcctcccgatcggcttcgtagatcagac

SEQ ID NO: 193 - RNA, 34 nt, synthetic construct
  source 1..34: mol_type = other RNA
gtctgatctacgaagccgatcgggaggctctgga

SEQ ID NO: 194 - RNA, 34 nt, synthetic construct
  source 1..34: mol_type = other RNA
tccagagccttccgatcggcttcgtagatcagac

SEQ ID NO: 195 - RNA, 34 nt, synthetic construct
  source 1..34: mol_type = other RNA
gtctgatctacgaagccgatcggaaggctctgga

SEQ ID NO: 196 - RNA, 34 nt, synthetic construct
  source 1..34: mol_type = other RNA
tccagagcctcccgatcggcttcataggtcggac

SEQ ID NO: 197 - RNA, 34 nt, synthetic construct
  source 1..34: mol_type = other RNA
gtccgacctatgaagccgatcgggaggctctgga

SEQ ID NO: 198 - RNA, 34 nt, synthetic construct
  source 1..34: mol_type = other RNA
tccagagccccctgatcggcttcgtagatcagac

SEQ ID NO: 199 - RNA, 34 nt, synthetic construct
  source 1..34: mol_type = other RNA
gtctgatctacgaagccgatcagggggctctgga

SEQ ID NO: 200 - RNA, 34 nt, synthetic construct
  source 1..34: mol_type = other RNA
tccaaagcctcccgatcggcttcgtagatcagac

SEQ ID NO: 201 - RNA, 34 nt, synthetic construct
  source 1..34: mol_type = other RNA
gtctgatctacgaagccgatcgggaggctttgga

SEQ ID NO: 202 - RNA, 34 nt, synthetic construct
  source 1..34: mol_type = other RNA
tccagagcctcctgttcggcttcgtagatcagaa

SEQ ID NO: 203 - RNA, 34 nt, synthetic construct
  source 1..34: mol_type = other RNA
ttctgatctacgaagccgaacaggaggctctgga

SEQ ID NO: 204 - RNA, 34 nt, synthetic construct
  source 1..34: mol_type = other RNA
tccagagcctcctgatcggcttcgtaggtcagac

SEQ ID NO: 205 - RNA, 34 nt, synthetic construct
  source 1..34: mol_type = other RNA
gtctgacctacgaagccgatcaggaggctctgga

SEQ ID NO: 206 - RNA, 30 nt, synthetic construct
  source 1..30: mol_type = other RNA
atttaaatagaaccatagtggaatgtaaat

SEQ ID NO: 207 - RNA, 30 nt, synthetic construct
  source 1..30: mol_type = other RNA
atttacattccactatggttctatttaaat

SEQ ID NO: 208 - RNA, 32 nt, synthetic construct
  source 1..32: mol_type = other RNA
ctttgcctccgcgcacacacacggattgcaac

SEQ ID NO: 209 - RNA, 32 nt, synthetic construct
  source 1..32: mol_type = other RNA
gttgcaatccgtgtgtgtgcgcggaggcaaag

SEQ ID NO: 210 - RNA, 32 nt, synthetic construct
  source 1..32: mol_type = other RNA
gtctgcctccgcgcacacacacggattgcaac

SEQ ID NO: 211 - RNA, 32 nt, synthetic construct
  source 1..32: mol_type = other RNA
gttgcaatccgtgtgtgtgcgcggaggcagac

SEQ ID NO: 212 - RNA, 17 nt, synthetic construct
  source 1..17: mol_type = other RNA
gaacatcctgaaccaca

SEQ ID NO: 213 - RNA, 17 nt, synthetic construct
  source 1..17: mol_type = other RNA
tgtggttcaggatgttc

SEQ ID NO: 214 - RNA, 23 nt, synthetic construct
  source 1..23: mol_type = other RNA
attaatttacatcattctcaaac

SEQ ID NO: 215 - RNA, 23 nt, synthetic construct
  source 1..23: mol_type = other RNA
gtttgagaatgatgtaaattaat

SEQ ID NO: 216 - RNA, 23 nt, synthetic construct
  source 1..23: mol_type = other RNA
ttcatattctatcattctcaaac

SEQ ID NO: 217 - RNA, 23 nt, synthetic construct
  source 1..23: mol_type = other RNA
gtttgagaatgatagaatatgaa

SEQ ID NO: 218 - RNA, 32 nt, synthetic construct
  source 1..32: mol_type = other RNA
gtctcaccttacacaggtgagtggattgaaat

SEQ ID NO: 219 - RNA, 32 nt, synthetic construct
  source 1..32: mol_type = other RNA
atttcaatccactcacctgtgtaaggtgagac

SEQ ID NO: 220 - RNA, 32 nt, synthetic construct
  source 1..32: mol_type = other RNA
gtctcaccttacataggtgagtggattgaaat

SEQ ID NO: 221 - RNA, 32 nt, synthetic construct
  source 1..32: mol_type = other RNA
atttcaatccactcacctatgtaaggtgagac

SEQ ID NO: 222 - RNA, 26 nt, synthetic construct
  source 1..26: mol_type = other RNA
tccacggcgttcagcagctagacatt

SEQ ID NO: 223 - RNA, 26 nt, synthetic construct
  source 1..26: mol_type = other RNA
aatgtctagctgctgaacgccgtgga

SEQ ID NO: 224 - RNA, 26 nt, synthetic construct
  source 1..26: mol_type = other RNA
tccacggcgtttagcagctagacatt

SEQ ID NO: 225 - RNA, 26 nt, synthetic construct
  source 1..26: mol_type = other RNA
aatgtctagctgctaaacgccgtgga

SEQ ID NO: 226 - RNA, 36 nt, synthetic construct
  source 1..36: mol_type = other RNA
gtttgagaatgatgtaaattaatatggttcttaatc

SEQ ID NO: 227 - RNA, 36 nt, synthetic construct
  source 1..36: mol_type = other RNA
gattaagaaccatattaatttacatcattctcaaac

SEQ ID NO: 228 - RNA, 36 nt, synthetic construct
  source 1..36: mol_type = other RNA
gtttgagaatgatgtaaatcaatatggttcttaatc

SEQ ID NO: 229 - RNA, 36 nt, synthetic construct
  source 1..36: mol_type = other RNA
gattaagaaccatattgatttacatcattctcaaac

SEQ ID NO: 230 - RNA, 36 nt, synthetic construct
  source 1..36: mol_type = other RNA
gtttgagaatgatgtaattcaatatggttacttaac

SEQ ID NO: 231 - RNA, 36 nt, synthetic construct
  source 1..36: mol_type = other RNA
gttaagtaaccatattgaattacatcattctcaaac

SEQ ID NO: 232 - RNA, 38 nt, synthetic construct
  source 1..38: mol_type = other RNA
agttacaatggcctctgccgtgtagcggcaagtggagg

SEQ ID NO: 233 - RNA, 38 nt, synthetic construct
  source 1..38: mol_type = other RNA
cctccacttgccgctacacggcagaggccattgtaact

SEQ ID NO: 234 - RNA, 38 nt, synthetic construct
  source 1..38: mol_type = other RNA
ggttacaatggcctctgccgtgtagcggcaagtggagg

SEQ ID NO: 235 - RNA, 38 nt, synthetic construct
  source 1..38: mol_type = other RNA
cctccacttgccgctacacggcagaggccattgtaacc

SEQ ID NO: 236 - RNA, 38 nt, synthetic construct
  source 1..38: mol_type = other RNA
cgttacaatggcctctgccgtgtagcggcaagtggagg

SEQ ID NO: 237 - RNA, 38 nt, synthetic construct
  source 1..38: mol_type = other RNA
cctccacttgccgctacacggcagaggccattgtaacg

SEQ ID NO: 238 - RNA, 38 nt, synthetic construct
  source 1..38: mol_type = other RNA
agttacaatggcctctgccgtgtagcggcaagtggagc

SEQ ID NO: 239 - RNA, 38 nt, synthetic construct
  source 1..38: mol_type = other RNA
gctccacttgccgctacacggcagaggccattgtaact

SEQ ID NO: 240 - RNA, 39 nt, synthetic construct
  source 1..39: mol_type = other RNA
gttacaatggcctctgccgtgtagcggcaagtggaggag

SEQ ID NO: 241 - RNA, 39 nt, synthetic construct
  source 1..39: mol_type = other RNA
ctcctccacttgccgctacacggcagaggccattgtaac

SEQ ID NO: 242 - RNA, 36 nt, synthetic construct
  source 1..36: mol_type = other RNA
gtctaatactttatattaatttctactattgtagat

SEQ ID NO: 243 - RNA, 36 nt, synthetic construct
  source 1..36: mol_type = other RNA
atctacaatagtagaaattaatataaagtattagac

SEQ ID NO: 244 - RNA, 35 nt, synthetic construct
  source 1..35: mol_type = other RNA
tctaatactttatattaatttctactattgtagat

SEQ ID NO: 245 - RNA, 35 nt, synthetic construct
  source 1..35: mol_type = other RNA
atctacaatagtagaaattaatataaagtattaga

SEQ ID NO: 246 - RNA, 37 nt, synthetic construct
  source 1..37: mol_type = other RNA
gttgcaatggtcaacagagcgtcgtggcaagtggagg

SEQ ID NO: 247 - RNA, 37 nt, synthetic construct
  source 1..37: mol_type = other RNA
cctccacttgccacgacgctctgttgaccattgcaac

SEQ ID NO: 248 - RNA, 37 nt, synthetic construct
  source 1..37: mol_type = other RNA
gttgcaatgggcaccgtggcatcgcggcaagtggagg

SEQ ID NO: 249 - RNA, 37 nt, synthetic construct
  source 1..37: mol_type = other RNA
cctccacttgccgcgatgccacggtgcccattgcaac

SEQ ID NO: 250 - RNA, 37 nt, synthetic construct
  source 1..37: mol_type = other RNA
cctgccgaacacatccgatcggatctcctgttcgacc

SEQ ID NO: 251 - RNA, 37 nt, synthetic construct
  source 1..37: mol_type = other RNA
ggtcgaacaggagatccgatcggatgtgttcggcagg

SEQ ID NO: 252 - RNA, 37 nt, synthetic construct
  source 1..37: mol_type = other RNA
cctgccgaacacatccgatcggatctcctgttcgacg

SEQ ID NO: 253 - RNA, 37 nt, synthetic construct
  source 1..37: mol_type = other RNA
cgtcgaacaggagatccgatcggatgtgttcggcagg

SEQ ID NO: 254 - RNA, 37 nt, synthetic construct
  source 1..37: mol_type = other RNA
cctgccgaacacatccgatcggatctcctgttcgaca

SEQ ID NO: 255 - RNA, 37 nt, synthetic construct
  source 1..37: mol_type = other RNA
tgtcgaacaggagatccgatcggatgtgttcggcagg

SEQ ID NO: 256 - RNA, 36 nt, synthetic construct
  source 1..36: mol_type = other RNA
cctgccgaacacatccgatcggatctcctgttcgac

SEQ ID NO: 257 - RNA, 36 nt, synthetic construct
  source 1..36: mol_type = other RNA
gtcgaacaggagatccgatcggatgtgttcggcagg

SEQ ID NO: 258 - RNA, 37 nt, synthetic construct
  source 1..37: mol_type = other RNA
atccaaagcacctcaatccccgcgaggggattacgac

SEQ ID NO: 259 - RNA, 37 nt, synthetic construct
  source 1..37: mol_type = other RNA
gtcgtaatcccctcgcggggattgaggtgctttggat

SEQ ID NO: 260 - RNA, 24 nt, synthetic construct
  source 1..24: mol_type = other RNA
ggtaataacgatcagaattggaag

SEQ ID NO: 261 - RNA, 24 nt, synthetic construct
  source 1..24: mol_type = other RNA
cttccaattctgatcgttattacc

SEQ ID NO: 262 - RNA, 24 nt, synthetic construct
  source 1..24: mol_type = other RNA
ggtagtaacgatcagaatttgaaa

SEQ ID NO: 263 - RNA, 24 nt, synthetic construct
  source 1..24: mol_type = other RNA
tttcaaattctgatcgttactacc

SEQ ID NO: 264 - RNA, 24 nt, synthetic construct
  source 1..24: mol_type = other RNA
ctttcagttcttcctagttgttac

SEQ ID NO: 265 - RNA, 24 nt, synthetic construct
  source 1..24: mol_type = other RNA
gtaacaactaggaagaactgaaag

SEQ ID NO: 266 - RNA, 24 nt, synthetic construct
  source 1..24: mol_type = other RNA
cttccaattctgatcgttgttacc

SEQ ID NO: 267 - RNA, 24 nt, synthetic construct
  source 1..24: mol_type = other RNA
ggtaacaacgatcagaattggaag

SEQ ID NO: 268 - RNA, 23 nt, synthetic construct
  source 1..23: mol_type = other RNA
atctgcaacagcagaaattttac

SEQ ID NO: 269 - RNA, 23 nt, synthetic construct
  source 1..23: mol_type = other RNA
gtaaaatttctgctgttgcagat

SEQ ID NO: 270 - RNA, 23 nt, synthetic construct
  source 1..23: mol_type = other RNA
acctgcgatagcaggaattggat

SEQ ID NO: 271 - RNA, 23 nt, synthetic construct
  source 1..23: mol_type = other RNA
atccaattcctgctatcgcaggt

SEQ ID NO: 272 - RNA, 23 nt, synthetic construct
  source 1..23: mol_type = other RNA
atctgcaacagcagaaatttgat

SEQ ID NO: 273 - RNA, 23 nt, synthetic construct
  source 1..23: mol_type = other RNA
atcaaatttctgctgttgcagat

SEQ ID NO: 274 - RNA, 23 nt, synthetic construct
  source 1..23: mol_type = other RNA
atctgcaacagcagaaatttgga

SEQ ID NO: 275 - RNA, 23 nt, synthetic construct
  source 1..23: mol_type = other RNA
tccaaatttctgctgttgcagat

SEQ ID NO: 276 - RNA, 23 nt, synthetic construct
  source 1..23: mol_type = other RNA
atctgcaacagcagaaatttcgc

SEQ ID NO: 277 - RNA, 23 nt, synthetic construct
  source 1..23: mol_type = other RNA
gcgaaatttctgctgttgcagat

SEQ ID NO: 278 - RNA, 23 nt, synthetic construct
  source 1..23: mol_type = other RNA
atctgcaacagcagaaatttaca

SEQ ID NO: 279 - RNA, 23 nt, synthetic construct
  source 1..23: mol_type = other RNA
tgtaaatttctgctgttgcagat

SEQ ID NO: 280 - RNA, 23 nt, synthetic construct
  source 1..23: mol_type = other RNA
atctgcgatagcagaaatttcgc

SEQ ID NO: 281 - RNA, 23 nt, synthetic construct
  source 1..23: mol_type = other RNA
gcgaaatttctgctatcgcagat

SEQ ID NO: 282 - RNA, 23 nt, synthetic construct
  source 1..23: mol_type = other RNA
atctgcaacagcagaaattttta

SEQ ID NO: 283 - RNA, 23 nt, synthetic construct
  source 1..23: mol_type = other RNA
taaaaatttctgctgttgcagat

SEQ ID NO: 284 - RNA, 23 nt, synthetic construct
  source 1..23: mol_type = other RNA
atcttcaacagcagaaatttggc

SEQ ID NO: 285 - RNA, 23 nt, synthetic construct
  source 1..23: mol_type = other RNA
gccaaatttctgctgttgaagat

SEQ ID NO: 286 - RNA, 23 nt, synthetic construct
  source 1..23: mol_type = other RNA
atctgcaacagcagaaattggca

SEQ ID NO: 287 - RNA, 23 nt, synthetic construct
  source 1..23: mol_type = other RNA
tgccaatttctgctgttgcagat

SEQ ID NO: 288 - RNA, 23 nt, synthetic construct
  source 1..23: mol_type = other RNA
atctgcaacagcagaaatttgag

SEQ ID NO: 289 - RNA, 23 nt, synthetic construct
  source 1..23: mol_type = other RNA
ctcaaatttctgctgttgcagat

SEQ ID NO: 290 - RNA, 20 nt, synthetic construct
  source 1..20: mol_type = other RNA
atctgcaacagcagaaattt

SEQ ID NO: 291 - RNA, 20 nt, synthetic construct
  source 1..20: mol_type = other RNA
aaatttctgctgttgcagat

SEQ ID NO: 292 - RNA, 19 nt, synthetic construct
  source 1..19: mol_type = other RNA
atctgcaacagcagaaatt

SEQ ID NO: 293 - RNA, 19 nt, synthetic construct
  source 1..19: mol_type = other RNA
aatttctgctgttgcagat

SEQ ID NO: 294 - RNA, 25 nt, synthetic construct
  source 1..25: mol_type = other RNA
ctttcaatattctattgaaatcaac

SEQ ID NO: 295 - RNA, 25 nt, synthetic construct
  source 1..25: mol_type = other RNA
gttgatttcaatagaatattgaaag

SEQ ID NO: 296 - RNA, 25 nt, synthetic construct
  source 1..25: mol_type = other RNA
ctttcaatattctactgaaatcaac

SEQ ID NO: 297 - RNA, 25 nt, synthetic construct
  source 1..25: mol_type = other RNA
gttgatttcagtagaatattgaaag

SEQ ID NO: 298 - RNA, 25 nt, synthetic construct
  source 1..25: mol_type = other RNA
ctttcgatattcctctggaatcatt

SEQ ID NO: 299 - RNA, 25 nt, synthetic construct
  source 1..25: mol_type = other RNA
aatgattccagaggaatatcgaaag

SEQ ID NO: 300 - RNA, 25 nt, synthetic construct
  source 1..25: mol_type = other RNA
ctttcaatattctattgaaattggc

SEQ ID NO: 301 - RNA, 25 nt, synthetic construct
  source 1..25: mol_type = other RNA
gccaatttcaatagaatattgaaag

SEQ ID NO: 302 - RNA, 25 nt, synthetic construct
  source 1..25: mol_type = other RNA
ctttcaatattccattgaaatcaac

SEQ ID NO: 303 - RNA, 25 nt, synthetic construct
  source 1..25: mol_type = other RNA
gttgatttcaatggaatattgaaag

SEQ ID NO: 304 - RNA, 25 nt, synthetic construct
  source 1..25: mol_type = other RNA
ctttcaatattctattgaaaatcaa

SEQ ID NO: 305 - RNA, 25 nt, synthetic construct
  source 1..25: mol_type = other RNA
ttgattttcaatagaatattgaaag

SEQ ID NO: 306 - RNA, 25 nt, synthetic construct
  source 1..25: mol_type = other RNA
gcttcaattcttctgttttagattc

SEQ ID NO: 307 - RNA, 25 nt, synthetic construct
  source 1..25: mol_type = other RNA
gaatctaaaacagaagaattgaagc

SEQ ID NO: 308 - RNA, 23 nt, synthetic construct
  source 1..23: mol_type = other RNA
tgatttcagaggaatattgaaac

SEQ ID NO: 309 - RNA, 23 nt, synthetic construct
  source 1..23: mol_type = other RNA
gtttcaatattcctctgaaatca

SEQ ID NO: 310 - RNA, 24 nt, synthetic construct
  source 1..24: mol_type = other RNA
ctttcagtcttatagaattgcttc

SEQ ID NO: 311 - RNA, 24 nt, synthetic construct
  source 1..24: mol_type = other RNA
gaagcaattctataagactgaaag

SEQ ID NO: 312 - RNA, 24 nt, synthetic construct
  source 1..24: mol_type = other RNA
ctttcagttttatagaattgcttc

SEQ ID NO: 313 - RNA, 24 nt, synthetic construct
  source 1..24: mol_type = other RNA
gaagcaattctataaaactgaaag

SEQ ID NO: 314 - RNA, 24 nt, synthetic construct
  source 1..24: mol_type = other RNA
ttttggtttttatagaattgcttc

SEQ ID NO: 315 - RNA, 24 nt, synthetic construct
  source 1..24: mol_type = other RNA
gaagcaattctataaaaaccaaaa

SEQ ID NO: 316 - RNA, 24 nt, synthetic construct
  source 1..24: mol_type = other RNA
ctttcattcttatagaattgcttc

SEQ ID NO: 317 - RNA, 24 nt, synthetic construct
  source 1..24: mol_type = other RNA
gaagcaattctataagaatgaaag

SEQ ID NO: 318 - RNA, 24 nt, synthetic construct
  source 1..24: mol_type = other RNA
ttttcagtcttatagaattgcttc

SEQ ID NO: 319 - RNA, 24 nt, synthetic construct
  source 1..24: mol_type = other RNA
gaagcaattctataagactgaaaa

SEQ ID NO: 320 - RNA, 24 nt, synthetic construct
  source 1..24: mol_type = other RNA
gtaacaactgaaagaaactaaaac

SEQ ID NO: 321 - RNA, 24 nt, synthetic construct
  source 1..24: mol_type = other RNA
gttttagtttctttcagttgttac

SEQ ID NO: 322 - RNA, 24 nt, synthetic construct
  source 1..24: mol_type = other RNA
ctttcaattctctatttgagatgc

SEQ ID NO: 323 - RNA, 24 nt, synthetic construct
  source 1..24: mol_type = other RNA
gcatctcaaatagagaattgaaag

SEQ ID NO: 324 - RNA, 32 nt, synthetic construct
  source 1..32: mol_type = other RNA
gtcgcacctctcgtaggtgcgtggattgaaat

SEQ ID NO: 325 - RNA, 32 nt, synthetic construct
  source 1..32: mol_type = other RNA
atttcaatccacgcacctacgagaggtgcgac

SEQ ID NO: 326 - RNA, 24 nt, synthetic construct
  source 1..24: mol_type = other RNA
ccatcagtatcaaagtaataccaa

SEQ ID NO: 327 - RNA, 24 nt, synthetic construct
  source 1..24: mol_type = other RNA
ttggtattactttgatactgatgg

SEQ ID NO: 328 - RNA, 25 nt, synthetic construct
  source 1..25: mol_type = other RNA
ggtactgcatctcatgctgaaggtg

SEQ ID NO: 329 - RNA, 25 nt, synthetic construct
  source 1..25: mol_type = other RNA
caccttcagcatgagatgcagtacc

SEQ ID NO: 330 - RNA, 25 nt, synthetic construct
  source 1..25: mol_type = other RNA
ggaaactctgcacatgctgaaggcg

SEQ ID NO: 331 - RNA, 25 nt, synthetic construct
  source 1..25: mol_type = other RNA
cgccttcagcatgtgcagagtttcc

SEQ ID NO: 332 - RNA, 25 nt, synthetic construct
  source 1..25: mol_type = other RNA
ggtagtgcatctcatgctgaaggtg

SEQ ID NO: 333 - RNA, 25 nt, synthetic construct
  source 1..25: mol_type = other RNA
caccttcagcatgagatgcactacc

SEQ ID NO: 334 - RNA, 25 nt, synthetic construct
  source 1..25: mol_type = other RNA
ggatacgcatctcatgcagagggtg

SEQ ID NO: 335 - RNA, 25 nt, synthetic construct
  source 1..25: mol_type = other RNA
caccctctgcatgagatgcgtatcc

SEQ ID NO: 336 - RNA, 25 nt, synthetic construct
  source 1..25: mol_type = other RNA
aatactcatgcacatgctgaaggtg

SEQ ID NO: 337 - RNA, 25 nt, synthetic construct
  source 1..25: mol_type = other RNA
caccttcagcatgtgcatgagtatt

SEQ ID NO: 338 - RNA, 25 nt, synthetic construct
  source 1..25: mol_type = other RNA
ggaaattactctcatgcggaaggtg

SEQ ID NO: 339 - RNA, 25 nt, synthetic construct
  source 1..25: mol_type = other RNA
caccttccgcatgagagtaatttcc

SEQ ID NO: 340 - RNA, 25 nt, synthetic construct
  source 1..25: mol_type = other RNA
aaacacgcatctcatgcagaaggtg

SEQ ID NO: 341 - RNA, 25 nt, synthetic construct
  source 1..25: mol_type = other RNA
caccttctgcatgagatgcgtgttt

SEQ ID NO: 342 - RNA, 25 nt, synthetic construct
  source 1..25: mol_type = other RNA
gctaatcacgcacatgcagaaggtg

SEQ ID NO: 343 - RNA, 25 nt, synthetic construct
  source 1..25: mol_type = other RNA
caccttctgcatgtgcgtgattagc

SEQ ID NO: 344 - RNA, 25 nt, synthetic construct
  source 1..25: mol_type = other RNA
ggtaaagcatcacacgcagaaggcg

SEQ ID NO: 345 - RNA, 25 nt, synthetic construct
  source 1..25: mol_type = other RNA
cgccttctgcgtgtgatgctttacc

SEQ ID NO: 346 - RNA, 25 nt, synthetic construct
  source 1..25: mol_type = other RNA
ggaagtttttctcatactcaaggta

SEQ ID NO: 347 - RNA, 25 nt, synthetic construct
  source 1..25: mol_type = other RNA
taccttgagtatgagaaaaacttcc

SEQ ID NO: 348 - RNA, 25 nt, synthetic construct
  source 1..25: mol_type = other RNA
ggtaatgcatctcatgctgaaggtg

SEQ ID NO: 349 - RNA, 25 nt, synthetic construct
  source 1..25: mol_type = other RNA
caccttcagcatgagatgcattacc

SEQ ID NO: 350 - RNA, 30 nt, synthetic construct
  source 1..30: mol_type = other RNA
gtttcagacggacccttgggcggttgaagc

SEQ ID NO: 351 - RNA, 30 nt, synthetic construct
  source 1..30: mol_type = other RNA
gcttcaaccgcccaagggtccgtctgaaac

SEQ ID NO: 352 - RNA, 30 nt, synthetic construct
  source 1..30: mol_type = other RNA
tttcagacggatcccttgggcggttgaagc

SEQ ID NO: 353 - RNA, 30 nt, synthetic construct
  source 1..30: mol_type = other RNA
gcttcaaccgcccaagggatccgtctgaaa

SEQ ID NO: 354 - RNA, 30 nt, synthetic construct
  source 1..30: mol_type = other RNA
gtttcagacggcctctggagtggccgaagc

SEQ ID NO: 355 - RNA, 30 nt, synthetic construct
  source 1..30: mol_type = other RNA
gcttcggccactccagaggccgtctgaaac

SEQ ID NO: 356 - RNA, 35 nt, synthetic construct
  source 1..35: mol_type = other RNA
gtttcaaccgcacgaggatccgtctgaaaccatgg

SEQ ID NO: 357 - RNA, 35 nt, synthetic construct
  source 1..35: mol_type = other RNA
ccatggtttcagacggatcctcgtgcggttgaaac

SEQ ID NO: 358 - RNA, 35 nt, synthetic construct
  source 1..35: mol_type = other RNA
gtttcaaccgcacgaggatccgtctgaaacatgga

SEQ ID NO: 359 - RNA, 35 nt, synthetic construct
  source 1..35: mol_type = other RNA
tccatgtttcagacggatcctcgtgcggttgaaac

SEQ ID NO: 360 - RNA, 28 nt, synthetic construct
  source 1..28: mol_type = other RNA
acccagacctcgacgccgactgccacgc

SEQ ID NO: 361 - RNA, 28 nt, synthetic construct
  source 1..28: mol_type = other RNA
gcgtggcagtcggcgtcgaggtctgggt

SEQ ID NO: 362 - RNA, 24 nt, synthetic construct
  source 1..24: mol_type = other RNA
gtcgccgaactcgtcgtcaccgcc

SEQ ID NO: 363 - RNA, 24 nt, synthetic construct
  source 1..24: mol_type = other RNA
ggcggtgacgacgagttcggcgac

SEQ ID NO: 364 - RNA, 30 nt, synthetic construct
  source 1..30: mol_type = other RNA
catttcaattccataaggtacaattaatac

SEQ ID NO: 365 - RNA, 30 nt, synthetic construct
  source 1..30: mol_type = other RNA
gtattaattgtaccttatggaattgaaatg

SEQ ID NO: 366 - RNA, 29 nt, synthetic construct
  source 1..29: mol_type = other RNA
gtattaattgtaccttatggaattgaaat

SEQ ID NO: 367 - RNA, 29 nt, synthetic construct
  source 1..29: mol_type = other RNA
atttcaattccataaggtacaattaatac

SEQ ID NO: 368 - RNA, 29 nt, synthetic construct
  source 1..29: mol_type = other RNA
gtattaattgtaccttatggaattaggac

SEQ ID NO: 369 - RNA, 29 nt, synthetic construct
  source 1..29: mol_type = other RNA
gtcctaattccataaggtacaattaatac

SEQ ID NO: 370 - RNA, 29 nt, synthetic construct
  source 1..29: mol_type = other RNA
gtattaattataccttatggaattgaaat

SEQ ID NO: 371 - RNA, 29 nt, synthetic construct
  source 1..29: mol_type = other RNA
atttcaattccataaggtataattaatac

SEQ ID NO: 372 - RNA, 54 nt, synthetic construct
  source 1..54: mol_type = other RNA
tttttctttgtcggggtagcgggattcgaacccacgaccccctgctcccaaagc

SEQ ID NO: 373 - RNA, 54 nt, synthetic construct
  source 1..54: mol_type = other RNA
gctttgggagcagggggtcgtgggttcgaatcccgctaccccgacaaagaaaaa

SEQ ID NO: 374 - RNA, 54 nt, synthetic construct
  source 1..54: mol_type = other RNA
tttttctttgtcggggtagcgggattcgaacccacgacccccagctcccaaagc

SEQ ID NO: 375 - RNA, 54 nt, synthetic construct
  source 1..54: mol_type = other RNA
gctttgggagctgggggtcgtgggttcgaatcccgctaccccgacaaagaaaaa

SEQ ID NO: 376 - RNA, 31 nt, synthetic construct
  source 1..31: mol_type = other RNA
tatttcaattccataaggtacaattaatact

SEQ ID NO: 377 - RNA, 31 nt, synthetic construct
  source 1..31: mol_type = other RNA
agtattaattgtaccttatggaattgaaata

SEQ ID NO: 378 - RNA, 31 nt, synthetic construct
  source 1..31: mol_type = other RNA
agtcctaattccataaggtacaattaatact

SEQ ID NO: 379 - RNA, 31 nt, synthetic construct
  source 1..31: mol_type = other RNA
agtattaattgtaccttatggaattaggact

SEQ ID NO: 380 - RNA, 29 nt, synthetic construct
  source 1..29: mol_type = other RNA
gtattaattgtaccttgtggaattgaaat

SEQ ID NO: 381 - RNA, 29 nt, synthetic construct
  source 1..29: mol_type = other RNA
atttcaattccacaaggtacaattaatac

SEQ ID NO: 382 - RNA, 29 nt, synthetic construct
  source 1..29: mol_type = other RNA
gtattaattgtacttatggaattgaaatt

SEQ ID NO: 383 - RNA, 29 nt, synthetic construct
  source 1..29: mol_type = other RNA
aatttcaattccataagtacaattaatac

SEQ ID NO: 384 - RNA, 30 nt, synthetic construct
  source 1..30: mol_type = other RNA
tgtattaattgtaccttatggaattgaaat

SEQ ID NO: 385 - RNA, 30 nt, synthetic construct
  source 1..30: mol_type = other RNA
atttcaattccataaggtacaattaataca

SEQ ID NO: 386 - RNA, 30 nt, synthetic construct
  source 1..30: mol_type = other RNA
ggtattaattgtaccttatggaattaggac

SEQ ID NO: 387 - RNA, 30 nt, synthetic construct
  source 1..30: mol_type = other RNA
gtcctaattccataaggtacaattaatacc

SEQ ID NO: 388 - RNA, 30 nt, synthetic construct
  source 1..30: mol_type = other RNA
ggtattaattgtaccttatggaattgaaat

SEQ ID NO: 389 - RNA, 30 nt, synthetic construct
  source 1..30: mol_type = other RNA
atttcaattccataaggtacaattaatacc

SEQ ID NO: 390 - RNA, 30 nt, synthetic construct
  source 1..30: mol_type = other RNA
agtattaattgtaccttatggaattgaaat

SEQ ID NO: 391 - RNA, 30 nt, synthetic construct
  source 1..30: mol_type = other RNA
atttcaattccataaggtacaattaatact

SEQ ID NO: 392 - RNA, 29 nt, synthetic construct
  source 1..29: mol_type = other RNA
gtattaattgtaccttatggatttgaaat

SEQ ID NO: 393 - RNA, 29 nt, synthetic construct
  source 1..29: mol_type = other RNA
atttcaaatccataaggtacaattaatac

SEQ ID NO: 394 - RNA, 29 nt, synthetic construct
  source 1..29: mol_type = other RNA
gtattaattgtatcttatggaattgaaat

SEQ ID NO: 395 - RNA, 29 nt, synthetic construct
  source 1..29: mol_type = other RNA
atttcaattccataagatacaattaatac

SEQ ID NO: 396 - RNA, 33 nt, synthetic construct
  source 1..33: mol_type = other RNA
aaagtattaattgtaccttatggaattgaaata

SEQ ID NO: 397 - RNA, 33 nt, synthetic construct
  source 1..33: mol_type = other RNA
tatttcaattccataaggtacaattaatacttt

SEQ ID NO: 398 - RNA, 24 nt, synthetic construct
  source 1..24: mol_type = other RNA
attcccaatatattgtgaatttga

SEQ ID NO: 399 - RNA, 24 nt, synthetic construct
  source 1..24: mol_type = other RNA
tcaaattcacaatatattgggaat

SEQ ID NO: 400 - RNA, 30 nt, synthetic construct
  source 1..30: mol_type = other RNA
gtttcaagcccttataggtattcaccaaac

SEQ ID NO: 401 - RNA, 30 nt, synthetic construct
  source 1..30: mol_type = other RNA
gtttggtgaatacctataagggcttgaaac

SEQ ID NO: 402 - RNA, 30 nt, synthetic construct
  source 1..30: mol_type = other RNA
tctcaaagcccttataggtattcatcaaac

SEQ ID NO: 403 - RNA, 30 nt, synthetic construct
  source 1..30: mol_type = other RNA
gtttgatgaatacctataagggctttgaga

SEQ ID NO: 404 - RNA, 30 nt, synthetic construct
  source 1..30: mol_type = other RNA
gtttcaagcccttataggcattcaccaaac

SEQ ID NO: 405 - RNA, 30 nt, synthetic construct
  source 1..30: mol_type = other RNA
gtttggtgaatgcctataagggcttgaaac

SEQ ID NO: 406 - RNA, 25 nt, synthetic construct
  source 1..25: mol_type = other RNA
gtttcccatgaaatgggtgttgaaa

SEQ ID NO: 407 - RNA, 25 nt, synthetic construct
  source 1..25: mol_type = other RNA
tttcaacacccatttcatgggaaac

SEQ ID NO: 408 - RNA, 47 nt, synthetic construct
  source 1..47: mol_type = other RNA
gttgttgcttatagtacaaaggtattaaaattcaagcaaatcacaac

SEQ ID NO: 409 - RNA, 47 nt, synthetic construct
  source 1..47: mol_type = other RNA
gttgtgatttgcttgaattttaatacctttgtactataagcaacaac

SEQ ID NO: 410 - RNA, 17 nt, synthetic construct
  source 1..17: mol_type = other RNA
cggcgactcagtcgccg

SEQ ID NO: 411 - RNA, 17 nt, synthetic construct
  source 1..17: mol_type = other RNA
cggcgactgagtcgccg

SEQ ID NO: 412 - RNA, 20 nt, synthetic construct
  source 1..20: mol_type = other RNA
aaacatcatacttattcaac

SEQ ID NO: 413 - RNA, 20 nt, synthetic construct
  source 1..20: mol_type = other RNA
gttgaataagtatgatgttt

SEQ ID NO: 414 - RNA, 36 nt, synthetic construct
  source 1..36: mol_type = other RNA
gtgcaacttgctgatgtttgtagtaccgaatggcgg

SEQ ID NO: 415 - RNA, 36 nt, synthetic construct
  source 1..36: mol_type = other RNA
ccgccattcggtactacaaacatcagcaagttgcac

SEQ ID NO: 416 - RNA, 36 nt, synthetic construct
  source 1..36: mol_type = other RNA
gtgcaacttgctgatgtttgtagtaccgaatggcga

SEQ ID NO: 417 - RNA, 36 nt, synthetic construct
  source 1..36: mol_type = other RNA
tcgccattcggtactacaaacatcagcaagttgcac

SEQ ID NO: 418 - RNA, 26 nt, synthetic construct
  source 1..26: mol_type = other RNA
agtaagaatttgcattggtaccaaac

SEQ ID NO: 419 - RNA, 26 nt, synthetic construct
  source 1..26: mol_type = other RNA
gtttggtaccaatgcaaattcttact

SEQ ID NO: 420 - RNA, 26 nt, synthetic construct
  source 1..26: mol_type = other RNA
agtaagaattggcacgggtaccaaac

SEQ ID NO: 421 - RNA, 26 nt, synthetic construct
  source 1..26: mol_type = other RNA
gtttggtacccgtgccaattcttact

SEQ ID NO: 422 - RNA, 26 nt, synthetic construct
  source 1..26: mol_type = other RNA
agtaagaatttacacggataccaaac

SEQ ID NO: 423 - RNA, 26 nt, synthetic construct
  source 1..26: mol_type = other RNA
gtttggtatccgtgtaaattcttact

SEQ ID NO: 424 - RNA, 26 nt, synthetic construct
  source 1..26: mol_type = other RNA
agtaagaattgtcacggggtaccaaa

SEQ ID NO: 425 - RNA, 26 nt, synthetic construct
  source 1..26: mol_type = other RNA
tttggtaccccgtgacaattcttact

SEQ ID NO: 426 - RNA, 26 nt, synthetic construct
  source 1..26: mol_type = other RNA
agcaagaatttacatgagttccaaac

SEQ ID NO: 427 - RNA, 26 nt, synthetic construct
  source 1..26: mol_type = other RNA
gtttggaactcatgtaaattcttgct

SEQ ID NO: 428 - RNA, 26 nt, synthetic construct
  source 1..26: mol_type = other RNA
agcaagaattgtcacgggtgccaaac

SEQ ID NO: 429 - RNA, 26 nt, synthetic construct
  source 1..26: mol_type = other RNA
gtttggcacccgtgacaattcttgct

SEQ ID NO: 430 - RNA, 26 nt, synthetic construct
  source 1..26: mol_type = other RNA
agtaagaatttacacgggtaccaaac

SEQ ID NO: 431 - RNA, 26 nt, synthetic construct
  source 1..26: mol_type = other RNA
gtttggtacccgtgtaaattcttact

SEQ ID NO: 432 - RNA, 26 nt, synthetic construct
  source 1..26: mol_type = other RNA
agtaaggatttacacgggtaccaaac

SEQ ID NO: 433 - RNA, 26 nt, synthetic construct
  source 1..26: mol_type = other RNA
gtttggtacccgtgtaaatccttact

SEQ ID NO: 434 - RNA, 26 nt, synthetic construct
  source 1..26: mol_type = other RNA
agtaagaatttacatgggtaccaaac

SEQ ID NO: 435 - RNA, 26 nt, synthetic construct
  source 1..26: mol_type = other RNA
gtttggtacccatgtaaattcttact

SEQ ID NO: 436 - RNA, 26 nt, synthetic construct
  source 1..26: mol_type = other RNA
agtaagaattgtcacggggcgccaaa

SEQ ID NO: 437 - RNA, 26 nt, synthetic construct
  source 1..26: mol_type = other RNA
tttggcgccccgtgacaattcttact

SEQ ID NO: 438 - RNA, 24 nt, synthetic construct
  source 1..24: mol_type = other RNA
ttgagtagtaagaatttacatggg

SEQ ID NO: 439 - RNA, 24 nt, synthetic construct
  source 1..24: mol_type = other RNA
cccatgtaaattcttactactcaa

SEQ ID NO: 440 - RNA, 24 nt, synthetic construct
  source 1..24: mol_type = other RNA
ttgagtagtaagaattgtcatggg

SEQ ID NO: 441 - RNA, 24 nt, synthetic construct
  source 1..24: mol_type = other RNA
cccatgacaattcttactactcaa

SEQ ID NO: 442 - RNA, 24 nt, synthetic construct
  source 1..24: mol_type = other RNA
ttgagcagtaagaatttacacggg

SEQ ID NO: 443 - RNA, 24 nt, synthetic construct
  source 1..24: mol_type = other RNA
cccgtgtaaattcttactgctcaa

SEQ ID NO: 444 - RNA, 24 nt, synthetic construct
  source 1..24: mol_type = other RNA
ttgagtagtaaaaattgttacggg

SEQ ID NO: 445 - RNA, 24 nt, synthetic construct
  source 1..24: mol_type = other RNA
cccgtaacaatttttactactcaa

SEQ ID NO: 446 - RNA, 24 nt, synthetic construct
  source 1..24: mol_type = other RNA
ttgagtagtaagaatatacacgag

SEQ ID NO: 447 - RNA, 24 nt, synthetic construct
  source 1..24: mol_type = other RNA
ctcgtgtatattcttactactcaa

SEQ ID NO: 448 - RNA, 24 nt, synthetic construct
  source 1..24: mol_type = other RNA
gttattgagtagtaagaatttaca

SEQ ID NO: 449 - RNA, 24 nt, synthetic construct
  source 1..24: mol_type = other RNA
tgtaaattcttactactcaataac

SEQ ID NO: 450 - RNA, 24 nt, synthetic construct
  source 1..24: mol_type = other RNA
gttgctgagtagcaagaaattaca

SEQ ID NO: 451 - RNA, 24 nt, synthetic construct
  source 1..24: mol_type = other RNA
tgtaatttcttgctactcagcaac

SEQ ID NO: 452 - RNA, 24 nt, synthetic construct
  source 1..24: mol_type = other RNA
gtttttgagcaataagaatttgca

SEQ ID NO: 453 - RNA, 24 nt, synthetic construct
  source 1..24: mol_type = other RNA
tgcaaattcttattgctcaaaaac

SEQ ID NO: 454 - RNA, 24 nt, synthetic construct
  source 1..24: mol_type = other RNA
gttgaagagccgcaagaattcgca

SEQ ID NO: 455 - RNA, 24 nt, synthetic construct
  source 1..24: mol_type = other RNA
tgcgaattcttgcggctcttcaac

SEQ ID NO: 456 - RNA, 23 nt, synthetic construct
  source 1..23: mol_type = other RNA
cttgaggcgctcgacctcctgtt

SEQ ID NO: 457 - RNA, 23 nt, synthetic construct
  source 1..23: mol_type = other RNA
aacaggaggtcgagcgcctcaag

SEQ ID NO: 458 - RNA, 23 nt, synthetic construct
  source 1..23: mol_type = other RNA
cttgagccgctcgacctgctgct

SEQ ID NO: 459 - RNA, 23 nt, synthetic construct
  source 1..23: mol_type = other RNA
agcagcaggtcgagcggctcaag

SEQ ID NO: 460 - RNA, 23 nt, synthetic construct
  source 1..23: mol_type = other RNA
gttgaggcgctcgacttcccgct

SEQ ID NO: 461 - RNA, 23 nt, synthetic construct
  source 1..23: mol_type = other RNA
agcgggaagtcgagcgcctcaac

SEQ ID NO: 462 - RNA, 23 nt, synthetic construct
  source 1..23: mol_type = other RNA
gttgaggtgctcgacctgctgct

SEQ ID NO: 463 - RNA, 23 nt, synthetic construct
  source 1..23: mol_type = other RNA
agcagcaggtcgagcacctcaac

SEQ ID NO: 464 - RNA, 23 nt, synthetic construct
  source 1..23: mol_type = other RNA
cttgaggcgctcgacctgctgct

SEQ ID NO: 465 - RNA, 23 nt, synthetic construct
  source 1..23: mol_type = other RNA
agcagcaggtcgagcgcctcaag

SEQ ID NO: 466 - RNA, 23 nt, synthetic construct
  source 1..23: mol_type = other RNA
cttgaggcgctcgacctcctgct

SEQ ID NO: 467 - RNA, 23 nt, synthetic construct
  source 1..23: mol_type = other RNA
agcaggaggtcgagcgcctcaag

SEQ ID NO: 468 - RNA, 23 nt, synthetic construct
  source 1..23: mol_type = other RNA
ctccaagcgctggacctcctcct

SEQ ID NO: 469 - RNA, 23 nt, synthetic construct
  source 1..23: mol_type = other RNA
aggaggaggtccagcgcttggag

SEQ ID NO: 470 - RNA, 23 nt, synthetic construct
  source 1..23: mol_type = other RNA
cttgaggcgctcgacctcctcct

SEQ ID NO: 471 - RNA, 23 nt, synthetic construct
  source 1..23: mol_type = other RNA
aggaggaggtcgagcgcctcaag

SEQ ID NO: 472 - RNA, 23 nt, synthetic construct
  source 1..23: mol_type = other RNA
gtcgagaagcttgagctgctgct

SEQ ID NO: 473 - RNA, 23 nt, synthetic construct
  source 1..23: mol_type = other RNA
agcagcagctcaagcttctcgac

SEQ ID NO: 474 - RNA, 23 nt, synthetic construct
  source 1..23: mol_type = other RNA
cctgagccgctccaccttctgct

SEQ ID NO: 475 - RNA, 23 nt, synthetic construct
  source 1..23: mol_type = other RNA
agcagaaggtggagcggctcagg

SEQ ID NO: 476 - RNA, 29 nt, synthetic construct
  source 1..29: mol_type = other RNA
cgtcccgcccacgcacgcgagggaacggc

SEQ ID NO: 477 - RNA, 29 nt, synthetic construct
  source 1..29: mol_type = other RNA
gccgttccctcgcgtgcgtgggcgggacg

SEQ ID NO: 478 - RNA, 29 nt, synthetic construct
  source 1..29: mol_type = other RNA
tgttccgcccacgcacgcgagggaacggc

SEQ ID NO: 479 - RNA, 29 nt, synthetic construct
  source 1..29: mol_type = other RNA
gccgttccctcgcgtgcgtgggcggaaca

SEQ ID NO: 480 - RNA, 29 nt, synthetic construct
  source 1..29: mol_type = other RNA
cgttccgcccacgcacgcgagggaacggc

SEQ ID NO: 481 - RNA, 29 nt, synthetic construct
  source 1..29: mol_type = other RNA
gccgttccctcgcgtgcgtgggcggaacg

SEQ ID NO: 482 - RNA, 29 nt, synthetic construct
  source 1..29: mol_type = other RNA
ggtctctcccacgcactcgagggaacgtc

SEQ ID NO: 483 - RNA, 29 nt, synthetic construct
  source 1..29: mol_type = other RNA
gacgttccctcgagtgcgtgggagagacc

SEQ ID NO: 484 - RNA, 28 nt, synthetic construct
  source 1..28: mol_type = other RNA
gtcccgcccacgcacgcgagggaacggc

SEQ ID NO: 485 - RNA, 28 nt, synthetic construct
  source 1..28: mol_type = other RNA
gccgttccctcgcgtgcgtgggcgggac

SEQ ID NO: 486 - RNA, 28 nt, synthetic construct
  source 1..28: mol_type = other RNA
gttccgcccacgcacgcgagggaacggc

SEQ ID NO: 487 - RNA, 28 nt, synthetic construct
  source 1..28: mol_type = other RNA
gccgttccctcgcgtgcgtgggcggaac

SEQ ID NO: 488 - RNA, 28 nt, synthetic construct
  source 1..28: mol_type = other RNA
gtctctcccacgcacgcgagggaacggc

SEQ ID NO: 489 - RNA, 28 nt, synthetic construct
  source 1..28: mol_type = other RNA
gccgttccctcgcgtgcgtgggagagac

SEQ ID NO: 490 - RNA, 28 nt, synthetic construct
  source 1..28: mol_type = other RNA
gtcccgcccacgcacacgagggaacggc

SEQ ID NO: 491 - RNA, 28 nt, synthetic construct
  source 1..28: mol_type = other RNA
gccgttccctcgtgtgcgtgggcgggac

SEQ ID NO: 492 - RNA, 28 nt, synthetic construct
  source 1..28: mol_type = other RNA
gcctcgcccacgcgtcctaggggacatc

SEQ ID NO: 493 - RNA, 28 nt, synthetic construct
  source 1..28: mol_type = other RNA
gatgtcccctaggacgcgtgggcgaggc

SEQ ID NO: 494 - RNA, 24 nt, synthetic construct
  source 1..24: mol_type = other RNA
gccgttccctcgcgtgcgtgggcg

SEQ ID NO: 495 - RNA, 24 nt, synthetic construct
  source 1..24: mol_type = other RNA
cgcccacgcacgcgagggaacggc

SEQ ID NO: 496 - RNA, 24 nt, synthetic construct
  source 1..24: mol_type = other RNA
gccgttccctcgcgtgcgtgggag

SEQ ID NO: 497 - RNA, 24 nt, synthetic construct
  source 1..24: mol_type = other RNA
ctcccacgcacgcgagggaacggc

SEQ ID NO: 498 - RNA, 28 nt, synthetic construct
  source 1..28: mol_type = other RNA
gtctcgcccacgcacacgaggggacggc

SEQ ID NO: 499 - RNA, 28 nt, synthetic construct
  source 1..28: mol_type = other RNA
gccgtcccctcgtgtgcgtgggcgagac

SEQ ID NO: 500 - RNA, 28 nt, synthetic construct
  source 1..28: mol_type = other RNA
gtctcgctcacgcacgcgagggaacggc

SEQ ID NO: 501 - RNA, 28 nt, synthetic construct
  source 1..28: mol_type = other RNA
gccgttccctcgcgtgcgtgagcgagac

SEQ ID NO: 502 - RNA, 28 nt, synthetic construct
  source 1..28: mol_type = other RNA
gtctcgcccacgcacacgagggggcgtc

SEQ ID NO: 503 - RNA, 28 nt, synthetic construct
  source 1..28: mol_type = other RNA
gacgccccctcgtgtgcgtgggcgagac

SEQ ID NO: 504 - RNA, 28 nt, synthetic construct
  source 1..28: mol_type = other RNA
gtcccgcccacgcacacgcacacgaggg

SEQ ID NO: 505 - RNA, 28 nt, synthetic construct
  source 1..28: mol_type = other RNA
ccctcgtgtgcgtgtgcgtgggcgggac

SEQ ID NO: 506 - RNA, 28 nt, synthetic construct
  source 1..28: mol_type = other RNA
cccacgcacacgcacacgaggggacggc

SEQ ID NO: 507 - RNA, 28 nt, synthetic construct
  source 1..28: mol_type = other RNA
gccgtcccctcgtgtgcgtgtgcgtggg

SEQ ID NO: 508 - RNA, 25 nt, synthetic construct
  source 1..25: mol_type = other RNA
ccgcccacgcacgcgagggaacggc

SEQ ID NO: 509 - RNA, 25 nt, synthetic construct
  source 1..25: mol_type = other RNA
gccgttccctcgcgtgcgtgggcgg

SEQ ID NO: 510 - RNA, 37 nt, synthetic construct
  source 1..37: mol_type = other RNA
gtcatcccccgcgagcgagcgggggccgcattgaagc

SEQ ID NO: 511 - RNA, 37 nt, synthetic construct
  source 1..37: mol_type = other RNA
gcttcaatgcggcccccgctcgctcgcgggggatgac

SEQ ID NO: 512 - RNA, 37 nt, synthetic construct
  source 1..37: mol_type = other RNA
gtcatcccccgcgagcgagcgggggccgcattgaagg

SEQ ID NO: 513 - RNA, 37 nt, synthetic construct
  source 1..37: mol_type = other RNA
ccttcaatgcggcccccgctcgctcgcgggggatgac

SEQ ID NO: 514 - RNA, 36 nt, synthetic construct
  source 1..36: mol_type = other RNA
gttgcaatcccaaggtaatgcgtctcccattgacgc

SEQ ID NO: 515 - RNA, 36 nt, synthetic construct
  source 1..36: mol_type = other RNA
gcgtcaatgggagacgcattaccttgggattgcaac

SEQ ID NO: 516 - RNA, 36 nt, synthetic construct
  source 1..36: mol_type = other RNA
gaattgatcccgttgtaatgcgtctcccattgacgc

SEQ ID NO: 517 - RNA, 36 nt, synthetic construct
  source 1..36: mol_type = other RNA
gcgtcaatgggagacgcattacaacgggatcaattc

SEQ ID NO: 518 - RNA, 36 nt, synthetic construct
  source 1..36: mol_type = other RNA
gcgtcaatgggagacgcattactttaggattgcaac

SEQ ID NO: 519 - RNA, 36 nt, synthetic construct
  source 1..36: mol_type = other RNA
gttgcaatcctaaagtaatgcgtctcccattgacgc

SEQ ID NO: 520 - RNA, 36 nt, synthetic construct
  source 1..36: mol_type = other RNA
gcgtcaatgggagacgcattactttgggattgcaac

SEQ ID NO: 521 - RNA, 36 nt, synthetic construct
  source 1..36: mol_type = other RNA
gttgcaatcccaaagtaatgcgtctcccattgacgc

SEQ ID NO: 522 - RNA, 36 nt, synthetic construct
  source 1..36: mol_type = other RNA
gcgtcaatgggagacgcattactttaggattgcatc

SEQ ID NO: 523 - RNA, 36 nt, synthetic construct
  source 1..36: mol_type = other RNA
gatgcaatcctaaagtaatgcgtctcccattgacgc

SEQ ID NO: 524 - RNA, 36 nt, synthetic construct
  source 1..36: mol_type = other RNA
gcgtcaatgggagacgcattaactttgggattgcaa

SEQ ID NO: 525 - RNA, 36 nt, synthetic construct
  source 1..36: mol_type = other RNA
ttgcaatcccaaagttaatgcgtctcccattgacgc

SEQ ID NO: 526 - RNA, 36 nt, synthetic construct
  source 1..36: mol_type = other RNA
gcgtcaatgggagacgcattactttggaattgcaac

SEQ ID NO: 527 - RNA, 36 nt, synthetic construct
  source 1..36: mol_type = other RNA
gttgcaattccaaagtaatgcgtctcccattgacgc

SEQ ID NO: 528 - RNA, 23 nt, synthetic construct
  source 1..23: mol_type = other RNA
ggcgtcaatgggagacgcattac

SEQ ID NO: 529 - RNA, 23 nt, synthetic construct
  source 1..23: mol_type = other RNA
gtaatgcgtctcccattgacgcc

SEQ ID NO: 530 - RNA, 23 nt, synthetic construct
  source 1..23: mol_type = other RNA
agcgtcaatgggagacgcattac

SEQ ID NO: 531 - RNA, 23 nt, synthetic construct
  source 1..23: mol_type = other RNA
gtaatgcgtctcccattgacgct

SEQ ID NO: 532 - RNA, 23 nt, synthetic construct
  source 1..23: mol_type = other RNA
ggcgtcaatgggagacgcattaa

SEQ ID NO: 533 - RNA, 23 nt, synthetic construct
  source 1..23: mol_type = other RNA
ttaatgcgtctcccattgacgcc

SEQ ID NO: 534 - RNA, 36 nt, synthetic construct
  source 1..36: mol_type = other RNA
atcgcaacatctcagaaacccgtcctaagttgacgg

SEQ ID NO: 535 - RNA, 36 nt, synthetic construct
  source 1..36: mol_type = other RNA
ccgtcaacttaggacgggtttctgagatgttgcgat

SEQ ID NO: 536 - RNA, 36 nt, synthetic construct
  source 1..36: mol_type = other RNA
ctcgcaacatctcagaaacccgtcctaagttgacgg

SEQ ID NO: 537 - RNA, 36 nt, synthetic construct
  source 1..36: mol_type = other RNA
ccgtcaacttaggacgggtttctgagatgttgcgag

SEQ ID NO: 538 - RNA, 36 nt, synthetic construct
  source 1..36: mol_type = other RNA
ctcgcaacatctcagaaagccgtcctaagttgacgg

SEQ ID NO: 539 - RNA, 36 nt, synthetic construct
  source 1..36: mol_type = other RNA
ccgtcaacttaggacggctttctgagatgttgcgag

SEQ ID NO: 540 - RNA, 36 nt, synthetic construct
  source 1..36: mol_type = other RNA
atcgcaacatcttagaaacccgtcctaagttgacgg

SEQ ID NO: 541 - RNA, 36 nt, synthetic construct
  source 1..36: mol_type = other RNA
ccgtcaacttaggacgggtttctaagatgttgcgat

SEQ ID NO: 542 - RNA, 36 nt, synthetic construct
  source 1..36: mol_type = other RNA
ctcgaaccatctcagaaatccgtcctaagctgagga

SEQ ID NO: 543 - RNA, 36 nt, synthetic construct
  source 1..36: mol_type = other RNA
tcctcagcttaggacggatttctgagatggttcgag

SEQ ID NO: 544 - RNA, 35 nt, synthetic construct
  source 1..35: mol_type = other RNA
tcgcaacatctcagaaacccgtcctaagttgacgg

SEQ ID NO: 545 - RNA, 35 nt, synthetic construct
  source 1..35: mol_type = other RNA
ccgtcaacttaggacgggtttctgagatgttgcga

SEQ ID NO: 546 - RNA, 35 nt, synthetic construct
  source 1..35: mol_type = other RNA
tcgcaacatctcagaaagccgtcctaagttgacgg

SEQ ID NO: 547 - RNA, 35 nt, synthetic construct
  source 1..35: mol_type = other RNA
ccgtcaacttaggacggctttctgagatgttgcga

SEQ ID NO: 548 - RNA, 35 nt, synthetic construct
  source 1..35: mol_type = other RNA
tcgcaacatcttagaaacccgtcctaagttgacgg

SEQ ID NO: 549 - RNA, 35 nt, synthetic construct
  source 1..35: mol_type = other RNA
ccgtcaacttaggacgggtttctaagatgttgcga

SEQ ID NO: 550 - RNA, 35 nt, synthetic construct
  source 1..35: mol_type = other RNA
tcgaaccatctcagaaatccgtcctaagctgagga

SEQ ID NO: 551 - RNA, 35 nt, synthetic construct
  source 1..35: mol_type = other RNA
tcctcagcttaggacggatttctgagatggttcga

SEQ ID NO: 552 - RNA, 36 nt, synthetic construct
  source 1..36: mol_type = other RNA
gtgcattgaccgctccctgacggaaccagccagcac

SEQ ID NO: 553 - RNA, 36 nt, synthetic construct
  source 1..36: mol_type = other RNA
gtgctggctggttccgtcagggagcggtcaatgcac

SEQ ID NO: 554 - RNA, 36 nt, synthetic construct
  source 1..36: mol_type = other RNA
gtgccttggccgacccctgacggaaccggccagcac

SEQ ID NO: 555 - RNA, 36 nt, synthetic construct
  source 1..36: mol_type = other RNA
gtgctggccggttccgtcaggggtcggccaaggcac

SEQ ID NO: 556 - RNA, 36 nt, synthetic construct
  source 1..36: mol_type = other RNA
gtgctttagttggcccctgacggaaccgaccagcac

SEQ ID NO: 557 - RNA, 36 nt, synthetic construct
  source 1..36: mol_type = other RNA
gtgctggtcggttccgtcaggggccaactaaagcac

SEQ ID NO: 558 - RNA, 36 nt, synthetic construct
  source 1..36: mol_type = other RNA
gtgccttgatcgacccctgacggaaccgaccagcac

SEQ ID NO: 559 - RNA, 36 nt, synthetic construct
  source 1..36: mol_type = other RNA
gtgctggtcggttccgtcaggggtcgatcaaggcac

SEQ ID NO: 560 - RNA, 36 nt, synthetic construct
  source 1..36: mol_type = other RNA
gtgccttgaccggcccctgacggaaccggtcagcac

SEQ ID NO: 561 - RNA, 36 nt, synthetic construct
  source 1..36: mol_type = other RNA
gtgctgaccggttccgtcaggggccggtcaaggcac

SEQ ID NO: 562 - RNA, 36 nt, synthetic construct
  source 1..36: mol_type = other RNA
gcgccttgactgacccctgacggaaccggccagcac

SEQ ID NO: 563 - RNA, 36 nt, synthetic construct
  source 1..36: mol_type = other RNA
gtgctggccggttccgtcaggggtcagtcaaggcgc

SEQ ID NO: 564 - RNA, 36 nt, synthetic construct
  source 1..36: mol_type = other RNA
gcgccttgaccgacccctgacggaacctgccagcac

SEQ ID NO: 565 - RNA, 36 nt, synthetic construct
  source 1..36: mol_type = other RNA
gtgctggcaggttccgtcaggggtcggtcaaggcgc

SEQ ID NO: 566 - RNA, 36 nt, synthetic construct
  source 1..36: mol_type = other RNA
gtgccttgaccgccccctaatggaaccggtcagcac

SEQ ID NO: 567 - RNA, 36 nt, synthetic construct
  source 1..36: mol_type = other RNA
gtgctgaccggttccattagggggcggtcaaggcac

SEQ ID NO: 568 - RNA, 36 nt, synthetic construct
  source 1..36: mol_type = other RNA
gtgccttgaccgccccctaatggaaccggccagcac

SEQ ID NO: 569 - RNA, 36 nt, synthetic construct
  source 1..36: mol_type = other RNA
gtgctggccggttccattagggggcggtcaaggcac

SEQ ID NO: 570 - RNA, 36 nt, synthetic construct
  source 1..36: mol_type = other RNA
gtgccttgactgacccctaacggaaccagccagcac

SEQ ID NO: 571 - RNA, 36 nt, synthetic construct
  source 1..36: mol_type = other RNA
gtgctggctggttccgttaggggtcagtcaaggcac

SEQ ID NO: 572 - RNA, 36 nt, synthetic construct
  source 1..36: mol_type = other RNA
gtgccttgactgaccactgacggaaccagccagcac

SEQ ID NO: 573 - RNA, 36 nt, synthetic construct
  source 1..36: mol_type = other RNA
gtgctggctggttccgtcagtggtcagtcaaggcac

SEQ ID NO: 574 - RNA, 36 nt, synthetic construct
  source 1..36: mol_type = other RNA
gtgccttgactgacccctgacggaaccagccagcac

SEQ ID NO: 575 - RNA, 36 nt, synthetic construct
  source 1..36: mol_type = other RNA
gtgctggctggttccgtcaggggtcagtcaaggcac

SEQ ID NO: 576 - RNA, 36 nt, synthetic construct
  source 1..36: mol_type = other RNA
gtgccttggccgaccccttacggaaccggccagcac

SEQ ID NO: 577 - RNA, 36 nt, synthetic construct
  source 1..36: mol_type = other RNA
gtgctggccggttccgtaaggggtcggccaaggcac

SEQ ID NO: 578 - RNA, 53 nt, synthetic construct
  source 1..53: mol_type = other RNA
gaagccctctgggacaatgaaggcggtgccagccccggcaagggcggcaaggc

SEQ ID NO: 579 - RNA, 53 nt, synthetic construct
  source 1..53: mol_type = other RNA
gccttgccgcccttgccggggctggcaccgccttcattgtcccagagggcttc

SEQ ID NO: 580 - RNA, 23 nt, synthetic construct
  source 1..23: mol_type = other RNA
tggccgccgggcatgccgatcat

SEQ ID NO: 581 - RNA, 23 nt, synthetic construct
  source 1..23: mol_type = other RNA
atgatcggcatgcccggcggcca

SEQ ID NO: 582 - RNA, 23 nt, synthetic construct
  source 1..23: mol_type = other RNA
tggcccccgggcatgccgatcat

SEQ ID NO: 583 - RNA, 23 nt, synthetic construct
  source 1..23: mol_type = other RNA
atgatcggcatgcccgggggcca

SEQ ID NO: 584 - RNA, 124 nt, synthetic construct
  source 1..124: mol_type = other RNA
ttctctcccggccagccttggctctcagctcaggccgatccagctcgaatccctgatagccacagatgagccggaaatccccgtccttcatcaactggagctttgcgccgcccggagcccacgc

SEQ ID NO: 585 - RNA, 124 nt, synthetic construct
  source 1..124: mol_type = other RNA
tggctatgtcgagttcaagctgattgggaaaaaactcaagcaaaacgcaaaggggaatagcaagcgcggccttgatgcatcgcaatggtacatcatcaagcaactgcaatcgggtgcgtgggct

SEQ ID NO: 586 - RNA, 79 nt, synthetic construct
  source 1..79: mol_type = other RNA
aacacatcgacattgctgcgccccctgaccgtggcatgcaacgcaccattctcgccgctctgcacattgatgctggctg

SEQ ID NO: 587 - RNA, 132 nt, synthetic construct
  source 1..132: mol_type = other RNA
aacgtagcagccgcgggtgaggatgtgaaggtgcggccagccggacttgtggagttccaggaacgccatgtattcgagtcgaggatagttgcggcggagccttcgcagcaaggccgtccagcgcccgcgcat

SEQ ID NO: 588 - RNA, 121 nt, synthetic construct
  source 1..121: mol_type = other RNA
ccgcgtccgactctccaaggcccgcgcccaggccgccgctggcaagccagagcgcatcatcacgctcactacacgcccccgcccaggtcttagcatcccctgtgccgtgcgctggatgcgc

SEQ ID NO: 589 - RNA, 133 nt, synthetic construct
  source 1..133: mol_type = other RNA
gcgatccgggagaaggagcaggtcttcgcgcagacgtggaaccgatcgacgctcgacagcctgcgcgacacgctctccgacggggtgctggagggcgagaacaaggaagagctcacggcccgcgtgcgcgagg

SEQ ID NO: 590 - RNA, 124 nt, synthetic construct
  source 1..124: mol_type = other RNA
tcctcgccccatatgtcccaagaccaaatgcgcagcttgcaggcgcgcgtgtcaatctcgacgcggcccggactgctgttcggcttgataccaggacagtcgatcctggccgagaagtccggcc

SEQ ID NO: 591 - RNA, 124 nt, synthetic construct
  source 1..124: mol_type = other RNA
cgaaagagcatggctaaaaaaccaccgaccattcgaaccacacttcagcacattctggataacgtgctcatcgccaaaaccagagcagagggcgacggcgtgattacgctggtccggccggact

SEQ ID NO: 592 - RNA, 128 nt, synthetic construct
  source 1..128: mol_type = other RNA
gcgccccgatctgcggcgtttcgtcgacgcgcgtcagagcatggtgcacctgtacggcttgcagacctatgccgacatcatgagcgaattcgcatccggtgagcgctacgtaaatcgggtgtggtcgg

SEQ ID NO: 593 - RNA, 126 nt, synthetic construct
  source 1..126: mol_type = other RNA
ggacgacgcggccctgatcgcgggcggaggcgagaaaggtctcgaggttctgctgcacgacgcggtagagaacggtgtcctcgggcttgcgccgctggtacccgagcgcaccggcggggtggtcgg

SEQ ID NO: 594 - RNA, 128 nt, synthetic construct
  source 1..128: mol_type = other RNA
ggagtgaccaaagtgttgtttgccgcggaccgcaaccgtgactgggcattccggaattgctcgacggccttaccgagataggtctcggcctccccgtcgtaaccatcggcagaagccgaccacacccg

SEQ ID NO: 595 - RNA, 126 nt, synthetic construct
  source 1..126: mol_type = other RNA
aatgatgttcttacctacaggttttagcctgaactgtgcataaaaactggcaacaccatacatgtcgctaagttttaagtttgtttccttttcaagcttcaggaacgcatcacgcggtataaaacc

SEQ ID NO: 596 - RNA, 126 nt, synthetic construct
  source 1..126: mol_type = other RNA
agcggaggcttacaacaatcgaggcaacacactacatgctcttcagcaatatcaggcggccctggatagctatgacaaggccatccatctcaagccggagtttgcggatgcctattccaaccgagc

SEQ ID NO: 597 - RNA, 126 nt, synthetic construct
  source 1..126: mol_type = other RNA
atgagggcaaacaggatgaagccggcgaacacaacatcgagcttgtcgaagcgggagaagatgcggcggtagcctttgagccgacggaagaggcgttcgatttcatttctgcggcggtataaaacc

SEQ ID NO: 598 - RNA, 129 nt, synthetic construct
  source 1..129: mol_type = other RNA
caaggttaccaacgcctttcagccaccggggcgctcctgccggctgcgcttcgtccgtctttccccgaaattgccgtcgattgtggcgatctccccgagaggcttgctctgcgggggttggagtcagcc

SEQ ID NO: 599 - RNA, 68 nt, synthetic construct
  source 1..68: mol_type = other RNA
cagaacaccgttccagccgcgcccagaaccacggcgcggatttcgcggttgcggccgatttcctccag

SEQ ID NO: 600 - RNA, 125 nt, synthetic construct
  source 1..125: mol_type = other RNA
cgcatgatctcatcgcgcttctcgcgggcagcctctccccccagcgcctcgaccatctcgtctcggaacagtcgccgatacacgtggcggatcgatccgggactccgggggaactcctcccgatc

SEQ ID NO: 601 - RNA, 125 nt, synthetic construct
  source 1..125: mol_type = other RNA
tcgctgccgtcagcacgaggagacgagcaggaagctccacgagtccctctccaagcatgagaaggatgctcggaaggccttcaagacgctgaacctcgacccctcgatcatctgggatcgggagg

SEQ ID NO: 602 - RNA, 139 nt, synthetic construct
  source 1..139: mol_type = other RNA
ctcccgatcggcttcgtagatcagacctgacgcagaaatggccggtagctcggtgtccgactccccatagcctcctgaatggcttcgtaattcagactggatctatcgccactgggcctcccgattggcttcgtagatc

SEQ ID NO: 603 - RNA, 125 nt, synthetic construct
  source 1..125: mol_type = other RNA
actcatgattaaagataaaagttctacagtaacacttgcaaaataatattataaaaaggagagtgaaactctccttaaaatttaagtaattatatttccgttattatctgtgaaagttctattta

SEQ ID NO: 604 - RNA, 124 nt, synthetic construct
  source 1..124: mol_type = other RNA
ttatttaaaggtcttaaagattttaactcagcttgtaaatcataaatatctttaaaaaaaacgctcatttcctactccttttatttactataattatttatatcatatcatataatatttaaat

SEQ ID NO: 605 - RNA, 124 nt, synthetic construct
  source 1..124: mol_type = other RNA
tatttaaaggtcttaaagattttaactcagcttgtaaatcataaatatctttaaaaaaaacgctcatttcctactccttttatttactataattatttatatcatatcatataatatttaaata

SEQ ID NO: 606 - RNA, 132 nt, synthetic construct
  source 1..132: mol_type = other RNA
acgcttggcatagcagagagcaccgtagagtcgcagatgcgcattgcgctgcgcacgttgcgtcgcagcttcgccgtgtaacagcccatgtcgcaagacggacaataaccccgaccacacacattgattgca

SEQ ID NO: 607 - RNA, 132 nt, synthetic construct
  source 1..132: mol_type = other RNA
tcaggctctctattcattgttgatgttgtctgcctgtggcagaacaggcggcgcaaggattgttatcggtgcggcgcagtgattgtgataatgtcctctttggtgttgtgttgtgtgcaatcaatgtgtgtg

SEQ ID NO: 608 - RNA, 123 nt, synthetic construct
  source 1..123: mol_type = other RNA
gttgcgagcatgggaatccgcagtgcgacccataagcgcgcagaataagagtatagcattcgcactgcaatcaatcggtcgattgtccgtccgaggctgtcgttaatgacatcataggatgtt

SEQ ID NO: 609 - RNA, 126 nt, synthetic construct
  source 1..126: mol_type = other RNA
cgataattccgcggcaccatcgctatttaacggcgattttgaaggtgcactaaatgaaagcttcgagcgtttccgttttggtggagtgacgccaagtaagcaagaagtgttctttattaatttaca

SEQ ID NO: 610 - RNA, 126 nt, synthetic construct
  source 1..126: mol_type = other RNA
atcggtaatcgcgatggagtagctaccatgagcgcgcacgaataattccgcattgtaaaaattatcgaaatagttgatcggttgctgcgtaccaaaacgaatgccgcgaatttcttgtaaattaat

SEQ ID NO: 611 - RNA, 132 nt, synthetic construct
  source 1..132: mol_type = other RNA
accaactcggcattcacgaactccacggcaccaaaatcgtaaaactagcagaataacccaaccaacctccctaaagcccctgcgaaaaggtcgcgcagaagcttggggaggttggtttacatcattctcaaa

SEQ ID NO: 612 - RNA, 124 nt, synthetic construct
  source 1..124: mol_type = other RNA
agtattgcgcggatataattttgtagatccttgatataatgcattttcaatcgtatatcgttcttgatacaaaatatgaagtgcccgatacacatcttccaattgataagtagattgatgtaaa

SEQ ID NO: 613 - RNA, 124 nt, synthetic construct
  source 1..124: mol_type = other RNA
gacgtttggctgctactgcttggttcgctcccttgccaccacctgcagaaaagatttcttttgtatgcatcgtttcaccaatttgtggcatatgacttaagcgaatcgtacggtctaaattaat

SEQ ID NO: 614 - RNA, 14 nt, synthetic construct
  source 1..14: mol_type = other RNA
tcttaaaatagcac

SEQ ID NO: 615 - RNA, 126 nt, synthetic construct
  source 1..126: mol_type = other RNA
cgataattccgcggcaccatcgctatttaacggcgattttgaaggcgcactgaatgaaagtttcgagcgtttccgttttggtggagtgacaccaagtaagcaagaagtgttctttattaatttaca

SEQ ID NO: 616 - RNA, 126 nt, synthetic construct
  source 1..126: mol_type = other RNA
atcggtaatcgcgatggagtagctaccatgagcacgcacgaataattccgcattgtaaaaattatcgaaatagttgatcggttgctgcgtaccaaaacgaatgcctcgtatttcttgtaaattaat

SEQ ID NO: 617 - RNA, 124 nt, synthetic construct
  source 1..124: mol_type = other RNA
gacgtttggctgctactgcttggttcgctcccttgccaccacctgcagaaaagatttcctttgtatgcatcgtttcaccaatttgtggcatatgacttaagcgaatcgtacggtctaaattaat

SEQ ID NO: 618 - RNA, 134 nt, synthetic construct
  source 1..134: mol_type = other RNA
aaacaacgattaaagcaagcaaggataaattaaccattgcttcttataatattgaaaacttctcagcaaataaagcgtccacaagtgatgaaaaagttcaacgcattgcccaatcgtttgtgaatgatttaaat

SEQ ID NO: 619 - RNA, 132 nt, synthetic construct
  source 1..132: mol_type = other RNA
cgcaggatacgctgtctgcagcgattgtcggcagtagtatcggttctacgaacgcccagagccatcggcgggtggcgtgccagaggcgccaatgcggtctggctggggctggcgcgtagtggcaagtggagg

SEQ ID NO: 620 - RNA, 132 nt, synthetic construct
  source 1..132: mol_type = other RNA
actcggcgctgctggtgctgcgcgtgctggaaacgctgggtgaggggtgaggggtgagaggcactggcgaatagtggcgaccccgactgggctcgaaccagtaaccttcagctcccctccacttgccactac

SEQ ID NO: 621 - RNA, 125 nt, synthetic construct
  source 1..125: mol_type = other RNA
ccgagttgcggcttgttagcccctcgcacagcgcgtgccagtcgcgctcgccggcccagatgcgccgcgtggcatcggcaatctgccagccgtttgcctcaagacgtgggaggcgctcctccact

SEQ ID NO: 622 - RNA, 124 nt, synthetic construct
  source 1..124: mol_type = other RNA
cgggataggacccgtggaattttgtgcttgccggagctctgaagaagtcgcagttaggcgagagcaagaactcgagaagcttatctgctccttctctcttgatcttatccttgaaaatttctac

SEQ ID NO: 623 - RNA, 124 nt, synthetic construct
  source 1..124: mol_type = other RNA
ataaggctttattaaaacaaaacactattatataagaaaaaaaggagaaaacattatgacaaacaataacattaacaacaacacaatcgatagagaagcggtacaggcccgtttcgtagaaatt

SEQ ID NO: 624 - RNA, 124 nt, synthetic construct
  source 1..124: mol_type = other RNA
agaaaaaacaacatgccgaattacgattacatcgacagacaaaagttagggataggcaaggcggacagaaatgcgtttgccgttcccgagtacgcagacggttggaacggcgcgatagaaatta

SEQ ID NO: 625 - RNA, 131 nt, synthetic construct
  source 1..131: mol_type = other RNA
cgcgagcgcaagggccgcgtgaaatgtccagggcctgtggccggacagcgtggaagcagtcggatcgatatctcgtcgctttgggacaaccaggccgagcagatgtgattgcgaaagcgtcgtggcaagtg

SEQ ID NO: 626 - RNA, 131 nt, synthetic construct
  source 1..131: mol_type = other RNA
cagagctgctacccgtgaggtagatggcagcacctgtagtatatgtcaacagctcgttgctggcaaccagacaagtcctgattatagaatagggattcgaacccccgacacccctcacttgccacgacgct

SEQ ID NO: 627 - RNA, 130 nt, synthetic construct
  source 1..130: mol_type = other RNA
gcgaggatccacggcaagttggtcgacaagctggacctgtcccgcgtcggcgacatgaagggcgacacgctccgccgcgaaatccgcgtggtcatcgaacacctctgcgacgccgacgacgcgctgttga

SEQ ID NO: 628 - RNA, 125 nt, synthetic construct
  source 1..125: mol_type = other RNA
cgcgggcggccttcagatccttcttggtgaaattcccgaccggcaggggcgtacacagggacagcacctcccggaagctccgcccgaactcgaccagttccttcgacggcttgccgttcggcagg

SEQ ID NO: 629 - RNA, 125 nt, synthetic construct
  source 1..125: mol_type = other RNA
ccgtgctggcggagttggccgggctggcggagccccccggccgtcctgccggggggctgctgacggtcggcgggttcgtgcggcggttcatggagcacgcgaaggtgtactaccgcctgccgaac

SEQ ID NO: 630 - RNA, 126 nt, synthetic construct
  source 1..126: mol_type = other RNA
catgtcccggagttgtcgtcgcaagtctttgatgtccgttcgctcccatttctcagtcctcttctggggttgaggtttcattcgatccggcccatttgtcatccaccagctcgaccccctcgcggg

SEQ ID NO: 631 - RNA, 124 nt, synthetic construct
  source 1..124: mol_type = other RNA
ttttaagcgatgcaagatacgctgatcaagtcgagaagtataacctattgaaaacagttaatccctcgaggataatcagcctactcgacagactagccgataacggaataattaataggaagaa

SEQ ID NO: 632 - RNA, 124 nt, synthetic construct
  source 1..124: mol_type = other RNA
tccaagtagcgagccaagggttccggaaaggtaggcaattggtactccgtaaaccgggttatcaacgagtagtattgttgttaaaacagctgttaatgaaggtatgaatcccgggacaacgatc

SEQ ID NO: 633 - RNA, 139 nt, synthetic construct
  source 1..139: mol_type = other RNA
acaaaaagtaataatccaggcatatctacgacaccgcctagcactttcagttcttcctagttgttacgcagaatcataggaaacattgaatggcttgaacacctagcagaacaatctttcagttcttcctagttgttac

SEQ ID NO: 634 - RNA, 135 nt, synthetic construct
  source 1..135: mol_type = other RNA
gaagctgggctacaccatcaagcagggcggcaagcgtgtctccgataagaccctaaaggaagctcaggacgacggtacaggacagttcatgctgggcctgtagcaaaacacctacaaatttctgctgttgcagat

SEQ ID NO: 635 - RNA, 134 nt, synthetic construct
  source 1..134: mol_type = other RNA
aagctgggctacaccatcaagcagggcggcaagcgtgtctccgataagaccctaaaggaagctcaggacgacggtacaggacagttcatgctgggcctgtagcaaaacacctacaaatttctgctgttgcagat

SEQ ID NO: 636 - RNA, 134 nt, synthetic construct
  source 1..134: mol_type = other RNA
tgagtggtcattccccgagaagggtctcgaaagattgctcctaatccctacaagatgtgcattttgtggggcaaaggagcgggggccatcgggtttccacgtaatatcctttaatatctgcaacagcagaaatt

SEQ ID NO: 637 - RNA, 124 nt, synthetic construct
  source 1..124: mol_type = other RNA
caagtggctaatgaactaacaaagacaccatcaatcataatcactgaggaacttggtgagaacccgcaggagtcgatgattgaggatgttgggaaggacgagttgaggcaccgcataaaacaga

SEQ ID NO: 638 - RNA, 124 nt, synthetic construct
  source 1..124: mol_type = other RNA
cgttgcgaataatgttacgggtgcgaataaactccagaggttattctacaataagtataggctggagtacccaagccttaactcacaattaatcatccaagcaatcaggcaggcatctgaaatc

SEQ ID NO: 639 - RNA, 124 nt, synthetic construct
  source 1..124: mol_type = other RNA
cttgtgaagtgtattgggcataccctactgtttctgtatgatgatacgtggaatagtttggatccgcgctccaaagccttatcctccacagccctcaccaacgccttgaatggtgtctgtttta

SEQ ID NO: 640 - RNA, 124 nt, synthetic construct
  source 1..124: mol_type = other RNA
acttttctgcttggaggatttgtcgccggaataccgatcattgtttacgatgaacctataaacgctttattcgagggaatatcggcaattacaacgaccggattatctagtttgaatcagaatt

SEQ ID NO: 641 - RNA, 124 nt, synthetic construct
  source 1..124: mol_type = other RNA
atattataggccgccgatcctggtgatactaggaacgatacagtaaggagcgctattccaagccctcctatccattggtaatatgatctgagaaaatgtactccaggggttagagaattctgat

SEQ ID NO: 642 - RNA, 124 nt, synthetic construct
  source 1..124: mol_type = other RNA
actcatagtaacccctgaccacttactcgtagacggggatactcttgaatggaaacaagcctttgaatttaagaaaggggataaagtactttcaatacaaaaaataccgggacacaacaacgat

SEQ ID NO: 643 - RNA, 129 nt, synthetic construct
  source 1..129: mol_type = other RNA
aaaagatcttcagaaaaataagtgagattctcaatacaaatctttcctcctcaaagctctgcgaaactctcatcacgatcagagatgttctcatgaagagtgatgatgatttcatcagaagatttgaag

SEQ ID NO: 644 - RNA, 124 nt, synthetic construct
  source 1..124: mol_type = other RNA
agtttataagcttcttaacaactattgttattgggatagtaaaaatatgatagacatggttaaagaaggcatcaaagaggatcttgtgaagctatatgacaacatatcctcatcatcagttgtt

SEQ ID NO: 645 - RNA, 125 nt, synthetic construct
  source 1..125: mol_type = other RNA
ctgatcgatgctcctataaggcttgaggttggcgatatgatatttctagaatgttcatctaaatatcttgagatccttaagaaggcatcggcatacagctcatatctatcgttcacatctcaaat

SEQ ID NO: 646 - RNA, 124 nt, synthetic construct
  source 1..124: mol_type = other RNA
gagagagatgctgagagattctgaaagctaagaaggcagtcccaagataatgttgaagacctgaagtcgagtagaacttcgggtttgaggctatgataggaaaatagaagtcctcaacaactga

SEQ ID NO: 647 - RNA, 64 nt, synthetic construct
  source 1..64: mol_type = other RNA
ggtacaaaaaccagttgtgtatccaaagcttcttctaaacatggcatcagagcctgatgatgtt

SEQ ID NO: 648 - RNA, 127 nt, synthetic construct
  source 1..127: mol_type = other RNA
cttgttttatacaatgagagcaatggctatgcagctatcaaagagcttgattatttaatgaatcttagaagtgcaaaagagagatttaagcaagtttatgacgagggatttgtagttgatactgatg

SEQ ID NO: 649 - RNA, 124 nt, synthetic construct
  source 1..124: mol_type = other RNA
tgttacctttatactaataccccttgtatgagctgcatctgtatataaagtataacgcccaccattaacatttaccttatttacaggctcttttgaattaccataataaaacttagtattactt

SEQ ID NO: 650 - RNA, 126 nt, synthetic construct
  source 1..126: mol_type = other RNA
cagaacctatttataaaatatcaattaattctgatttagatgtagataagctacctaaggctagagtatttgagcctgaagaattcaataaaggttggaaaactgtaaataactcttggtattact

SEQ ID NO: 651 - RNA, 123 nt, synthetic construct
  source 1..123: mol_type = other RNA
tcaacaataataatattattaatgtcagtaacaaatgcaccagcatagcttatattgctactaccaccaactaatactttaggcttttcaataaatttgcgtgcgtaaagaataccaccttca

SEQ ID NO: 652 - RNA, 123 nt, synthetic construct
  source 1..123: mol_type = other RNA
aagctatgctggtgcatttgttactgacattaataatattattattgttgatgctaatggctacatactgatagagggttaatgaatagaggtatgttttaaatgacacaactaaatgcatta

SEQ ID NO: 653 - RNA, 130 nt, synthetic construct
  source 1..130: mol_type = other RNA
aactgctgatgaccctgaccaaaagattcgttctaatggttatgtagttcaagttggggcatatggtaagagttctattatgacaggtgcaaaatctcaaaatgtaggtaagaagtctcatactgaaggt

SEQ ID NO: 654 - RNA, 127 nt, synthetic construct
  source 1..127: mol_type = other RNA
tctcggtatcgacgagtttcgtgacttttagcttctgcacgcggtagctaatacgctggcagtagtggcggcttgctgctgatcgttcgtagaatgtggcgtcgatagcagcatgttcagacggatc

SEQ ID NO: 655 - RNA, 127 nt, synthetic construct
  source 1..127: mol_type = other RNA
ggagatcggcctcagcgcggccgatctcccttcgccgtccacgttgtgtaaagcgttcgaccggatcgagatgagcgtctgtcgagtgctactgcgccagtcggcgcagttgcacgatccgtctgaa

SEQ ID NO: 656 - RNA, 127 nt, synthetic construct
  source 1..127: mol_type = other RNA
accggtcgatcgcgacgaggcctgcggcctccagatccgccacgtagcgatcgatactggcgtcggcgtagtcgatctgggtatctcgcttcagcgcccgcactgagcgctcgctgtcggcgtcgag

SEQ ID NO: 657 - RNA, 126 nt, synthetic construct
  source 1..126: mol_type = other RNA
acgcagcctcctggaggccatatcaaacagcacgcaccgccaacacgaccctacggcgttattttcgagatcgccaacgagaccgatttcttcgagatcccgcccgatcgctactacgagttcggc

SEQ ID NO: 658 - RNA, 66 nt, synthetic construct
  source 1..66: mol_type = other RNA
atgctgtcggcgctgttcgcgagcgatgagccactcgatcgacaggcgctcatcgaagtcacgagc

SEQ ID NO: 659 - RNA, 125 nt, synthetic construct
  source 1..125: mol_type = other RNA
tttcctaatacaagaatcactagtaatcagctcaaaagaaagcctaaagcatgaataggatgcaaattgtttgatatatagaaaaatcgctaacttatactaaagataaaagtatccttatggaa

SEQ ID NO: 660 - RNA, 130 nt, synthetic construct
  source 1..130: mol_type = other RNA
aaaacgcggaagaagcctaaactcctggttattatagtgatataatgaatcgttacgatattggcctccaaaccagaaaccaatgtccatcaccgtcccattatttagagatttgatttcatttccataa

SEQ ID NO: 661 - RNA, 127 nt, synthetic construct
  source 1..127: mol_type = other RNA
aaaatgacttagcatatttggatagtcgtatgagcgttttgattcaagaaatgttctcatctgatttgtgcgctgttgtgatgacgaaggatcctgtagaactagaagaagtctttggaattgaaat

SEQ ID NO: 662 - RNA, 129 nt, synthetic construct
  source 1..129: mol_type = other RNA
aaagagaataatgattacgggagctgccggttcaattgggcgtgaaatggtgaggcagatagccggattgaatccatataaattaattttggttgatcaggcagaatcacctttgcataatgtacaatt

SEQ ID NO: 663 - RNA, 118 nt, synthetic construct
  source 1..118: mol_type = other RNA
aaatttattgatatgtatgccggacgcctgcggaatcaagtctcttttatgttgagtgcctgcaacttctatatgcctatctttgaggaggcccttgatgcgtataatttaccattgg

SEQ ID NO: 664 - RNA, 126 nt, synthetic construct
  source 1..126: mol_type = other RNA
aaaaatagaatctttcaaagccaatgctaactctaaggcttgatcgagataaaacaaagcttccttttcttttccttcaacaacacgcacccatcccatattcgaaagagaatacattaatacttt

SEQ ID NO: 665 - RNA, 126 nt, synthetic construct
  source 1..126: mol_type = other RNA
aaacatagtggcaagccccatgcccataccattaatggccgaggaagtcgtaccgagtgtaggacacataccaagcaggagcacaaatgtcggattctctttaacaatcccattcattaatacttt

SEQ ID NO: 666 - RNA, 126 nt, synthetic construct
  source 1..126: mol_type = other RNA
aaagaagctgacgcctacaaatccctctttgaaaaaatgggtattgaaaacattgtctataacatagcaatgtgctccaaaaaccattattgcaaatatgaatctcaacgatgttaaagtattaat

SEQ ID NO: 667 - RNA, 126 nt, synthetic construct
  source 1..126: mol_type = other RNA
aagcatacttcttgattcatactaactatactttccatatatttgcagtattcaacacacacatcacatctttaatgatgaaaataaaaaaacgataagacaatacgaaattatgattaatacttt

SEQ ID NO: 668 - RNA, 102 nt, synthetic construct
  source 1..102: mol_type = other RNA
aaggaaatttattgttttgacgatttacaaattaattgtccgaaggaaccattcttcgggacaataagtcacaaatacccggaacatagttgtctatgtctc

SEQ ID NO: 669 - RNA, 126 nt, synthetic construct
  source 1..126: mol_type = other RNA
caaggggttaactttgctttatttccaatatatctacgactctccaaatcaaacatcttataccccaaaagagtgacatctatatcagttcgattatagacttcttgtaatgacaatttcaattcc

SEQ ID NO: 670 - RNA, 126 nt, synthetic construct
  source 1..126: mol_type = other RNA
cagataattgtgttttccgataccttcattgtctgccgacatattgaataggatttccgccccttgtaaagctagcgatgagctgggaggaatcgtggaccatagatcttcacaaatttcaattcc

SEQ ID NO: 671 - RNA, 126 nt, synthetic construct
  source 1..126: mol_type = other RNA
cagctattatccctgcaacaattcaaaaatatgtaacgaccaattatcccgatgctaaaatcctgaaaatagagagggataaaaaagattatgaagtaaaactgtccaaccgtacggaattgaaat

SEQ ID NO: 672 - RNA, 126 nt, synthetic construct
  source 1..126: mol_type = other RNA
cccaaatcatcttaccaagagaaccataatcattcttcaaggcgtctacatgattaccaccagccgcagctattacttcgtccttagatactggcaatcttcttggattattaccggaattgaaat

SEQ ID NO: 673 - RNA, 113 nt, synthetic construct
  source 1..113: mol_type = other RNA
cgtaaaccggcagacacacatgtaaattcgcgggtaaacacgttgacggatagatgcttccaaaagcgcatagcagcaccaatgttccaaccatgcgtatcatattccgccat

SEQ ID NO: 674 - RNA, 126 nt, synthetic construct
  source 1..126: mol_type = other RNA
ctttagaaataatgtgtcgatcaagaataatataaaaaacataggttagcgacataggtactaaacatctatatatcaacatgtcatagagcgtttccgttactttaccattgataattcacaata

SEQ ID NO: 675 - RNA, 125 nt, synthetic construct
  source 1..125: mol_type = other RNA
cctaaaccaatacagaaggtccttgaaacactgataaaatgccctagatgcggaagccaaccaagctatggagaaaaatactgcaaacaatgcggagcaagaatcccaacaaaagcatgggaaac

SEQ ID NO: 676 - RNA, 129 nt, synthetic construct
  source 1..129: mol_type = other RNA
cctctaccgaggttgccagtttagatgctgagcgcataacctccctctcaaaaaccctctcatccacatccaacaacgccagcctaatagcctctctcctgaaggcttcaagaggaacacccatatcat

SEQ ID NO: 677 - RNA, 129 nt, synthetic construct
  source 1..129: mol_type = other RNA
ggcgaaaaaccgcagccgcagcagccagctccgcctacaaggggggtaacggctcctgagcctgttcctgcaggggttgattgggagcctgatgaagacatggttgaggcttttgatgatatgggtgtt

SEQ ID NO: 678 - RNA, 126 nt, synthetic construct
  source 1..126: mol_type = other RNA
acgagtaagaaacgaacacgaggcctacggattgcattttgcggaagtttggttgcaactgcccgactacgacaactacgaactgcaagccaccgtcgtacttgacagtttgctcactgagtcgcc

SEQ ID NO: 679 - RNA, 125 nt, synthetic construct
  source 1..125: mol_type = other RNA
cctctgcagctgagattgttacaggtgcgatacaagaccaagacagaggtatggtggtgggaagacgttcctttggtaagggacttgtacagcgcccggttgagtttcaggatggaagtatgatg

SEQ ID NO: 680 - RNA, 124 nt, synthetic construct
  source 1..124: mol_type = other RNA
cgacgtacaatatcttctcttgacatcttcacccctgcgatagcggtatcgtttaccataacaatacggtcgccggcaagtatccccaccttctcagaaggtccgttggtaaccggttgaataa

SEQ ID NO: 681 - RNA, 129 nt, synthetic construct
  source 1..129: mol_type = other RNA
aacacattgtctgaacttcttctaattcctgttctgttgtaataaacaaagttggttcatttgttctgattttaatccatccaaaatcactataaatttcactacaagagaaataacaagcatcagcaa

SEQ ID NO: 682 - RNA, 129 nt, synthetic construct
  source 1..129: mol_type = other RNA
gaagtaggagttcctttatatggacctttaattaacacagtaactagaggcgcaagattaaagaaattctatctgcggtccgcagcttctggagtcggcaaatcaagaacaatgattgctgatgcttgt

SEQ ID NO: 683 - RNA, 129 nt, synthetic construct
  source 1..129: mol_type = other RNA
ttacggatttccacaacgtcgtctgttacaagacggtgaccgcgctttacaagttccaaagcagcctcagatttaccgatgccgctctcaccagtaatcaatacaccttcaccgtaaacatcaacaagt

SEQ ID NO: 684 - RNA, 125 nt, synthetic construct
  source 1..125: mol_type = other RNA
caccttatccatggcaatttgctcttcggaaagcgggccgagcggtttaccttgttcagaaggagaacggcgagcagcaaaggagaacagaccggcggaatcagcaggagcccctttgctgatgt

SEQ ID NO: 685 - RNA, 125 nt, synthetic construct
  source 1..125: mol_type = other RNA
gaatgtgcctacgctgcgaataaggcggtgggtgaggttttgcctgtaccccggactcagggtgaccgaggcggaaagaagtatctggtggaactgtggcccaccaagcagcaatacatcagcaa

SEQ ID NO: 686 - RNA, 124 nt, synthetic construct
  source 1..124: mol_type = other RNA
tcctgaaaaagacgatgcgtatcgtagatggcatcatagggagagtactgtgcattctgtcgcaaatcatcatcctcaaattcagcattgatgatgcgaccttggtaaattctgttgatgtttg

SEQ ID NO: 687 - RNA, 137 nt, synthetic construct
  source 1..137: mol_type = other RNA
agcttcattccgtggaagtgccccaaaaaggttgcacggcatagtcggatttggttcccgtgcaattcttgctactcaccagccggcgtgccccaccggatactcaggctccatatttggtgcccgtgcaaattctt

SEQ ID NO: 688 - RNA, 137 nt, synthetic construct
  source 1..137: mol_type = other RNA
gtcgttttcatagtataattggcgcgggcactcacagaatagtgctggataaaagagtggcgccttcctccttcttcgaaaaataagagatacggggtttgcgttgttgagtagcaagaatttgcacgggcaccaaa

SEQ ID NO: 689 - RNA, 124 nt, synthetic construct
  source 1..124: mol_type = other RNA
tgaaaaaattatttgtggttgtcaacacagatacccaacgggccattcgcgcttttgtgagcgagtcccgagcggatgattacgctgacgatctatacgaaaaaacgaaccaggatacccgtgt

SEQ ID NO: 690 - RNA, 127 nt, synthetic construct
  source 1..127: mol_type = other RNA
cgcgcgggaggcctggacggacaagatcgcctcggacaccgtcaagacggccctgcggctcggggtgagcgtcatcgcggtgtacaaggacgaggactccaccccctcgcaggtgcgtcccgcccac

SEQ ID NO: 691 - RNA, 127 nt, synthetic construct
  source 1..127: mol_type = other RNA
cccggtggccgacgcccttgctgacgaccttgacctcgatgccgtgcagcggcgccaccagggcgatctggtcggcgatcgcacggcgcgcctcggtggccagacgccggttgatgtgggcgggacg

SEQ ID NO: 692 - RNA, 125 nt, synthetic construct
  source 1..125: mol_type = other RNA
cgggaccttcagcaccgcggcggtcacccgccgctcctgggcaagggcgagggctgcgagcccgccgccgaagctgacgccgtggatgtcgatgcggcgcaggtcagggtacagggcgtgggcgg

SEQ ID NO: 693 - RNA, 125 nt, synthetic construct
  source 1..125: mol_type = other RNA
agcgtccagccggacgtgcccgacgtcgtggccgagcacgtcctgcacggcatcgcctcgcgcgagacgtacatcatcggcgggtgcgtccaaggcgtgtgggccgcggtctcagccgcccacgc

SEQ ID NO: 694 - RNA, 66 nt, synthetic construct
  source 1..66: mol_type = other RNA
aggtacatggccacgtggtagatcgcctggatgtccgtcttggtcgggtcggacgtgtagtagatc

SEQ ID NO: 695 - RNA, 131 nt, synthetic construct
  source 1..131: mol_type = other RNA
catggtgaaactggcagactcccactgaaccattgggaacaggatacccgtgctggccgagctttgccaggatagcaggcgccagcaactggccttttgctgtaggtataccgtccttcaatgcggccccc

SEQ ID NO: 696 - protein, 1139 aa, synthetic construct
IFVVLSVNQFRCNPNAFRQASMKTDDEKKDTAIKSYTSWLLPNDRKEKDMVRSFLALDQGSRFFFDLMQAWYGGLTPDIIKSVTKERDLVDLWCAIYWFRPMKKELASHPIDRVDLAKTFQRYYGGPASDVVQEYLSASIGEDCCWNDCRQKYQEFCKNLGVDFTPDLKTLVREKLIAVALNDGSQAAISNLFGNGGKEDRNVKVNICKKILIALEKAGKSVKYVRDVQSIILKCANAKDREDFNRIYADKNGRPGTLLLLLDRKGENTYDAEVLKRYLRKTIKSKNAPLVWNYNQKLFEYIERGIRNLLRRTINYDAWAWGEMWRVGHTPLQTKATRNYHFAQEQLKRHSDIAAASQSSHAIAVALNEFFESPFFDGENPFTICPHHLGKNLEKLFKAWADEKNQTDEEDAGETAITDYCAEFKDGFDREPIRNLLRYVYASLRSKYSAKELIQAAKYNQQFERYQRQKAHPVTPDNQGYTWPEAVIKPSKAERKNRENSLDTRIWVSVKLLQKDGTWEKHHIPFYNLRFFEEVYAAASIAEDMPSPTFRNSRFGFKLPKLLNTHAKINKKKKDGKKDKKAVNAAKREARIYLAAKEGRLPAPSVPLDKLSAVIARVNGKFQVTVPVKFKVNKQPKYPLPQLGQIILGYDQNLTANHAWALFEVVTEDTPGAHFCRNQYLRVLKTGQVRSITKTKDGQEIDQLSYDGLEYKEYGEWRKQAKKFASQWYITTKVRKGKETTLLSQPALERFESIEKYKPALYRFNKEYARLLLAVIRRKTLNELEEIRPEIFRLVEQGFGICRFGSLSLSSFEAIRAAKAVIYGYFSTALKHDHNTPITDDERQAFDPELFGLLNKLETLRQNKGEEKINRAANALIRIALENKADFIRGEGDLPTTNKSVKKGQNSRSMDWLARGLFNKICQLAVMHNIIPTAANPHSTSHQDPFEHNKDFDDPQPAMKCRYAEFKIEDIGDSVLERLGTSLRNAKAGQTGAYYYKGAQDFLAHYGLQDIEEELKKCRRGRKANMPCWELQKRLIQKLGSKDAVVYIPMRGGRIYLATHSVTTGAKPIIFNGEEVWVSNADEIAAVNIGLTIIPTSKKDEEHPDRENDDKAIRKSVRRPRKSGERRSGDKMPATARKT

SEQ ID NO: 697 - DNA, 9375 bp, synthetic construct
  source 1..9375: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacatcttcgtggtgctgagcgtgaatcagttcagatgcaaccccaacgccttcagacaagcctccatgaaaaccgacgacgagaagaaggacaccgccatcaagagctacacaagctggctgctgcccaacgacagaaaggagaaggacatggtgcggagcttcctggccctggaccaaggcagcagattctttttcgacctgatgcaagcctggtacggcggcctcacccccgacatcatcaagagcgtgaccaaggagcgggacctggtggacctgtggtgcgccatctactggttcagacccatgaagaaggagctggcctcccaccccatcgacagagtggacctggccaagacctttcagagatactacggcggccccgcctccgacgtggtgcaagagtacctgagcgcctccatcggcgaggactgctgctggaacgactgcagacagaagtaccaagagttctgcaagaacctcggggtggacttcacccccgacctcaagaccctggtgagagagaagctgatcgccgtggccctgaacgacggcagccaagccgccatcagcaacctgttcggcaacggcggcaaggaggacagaaacgtgaaggtgaacatctgcaagaagatcctgattgctctggagaaggccggcaaatccgtgaagtacgtgcgggacgtgcagagcatcatcctgaagtgcgccaacgccaaggacagagaggacttcaacagaatctacgccgacaagaacggcagacccggcaccctgctcctgctcctggacagaaagggcgagaacacctacgacgccgaggtgctgaagagatacctgagaaagaccatcaagagcaagaacgcccccctggtgtggaactacaatcagaagctgttcgagtacatcgagagaggcatccggaatctgctgagaagaaccatcaactacgacgcctgggcctggggcgagatgtggagagtgggccacacccccctgcagaccaaggccacaagaaactaccacttcgcccaagagcagctgaagagacacagcgacatcgccgccgcttcccaaagcagccacgccatcgccgtcgctctgaacgagttcttcgagagccccttcttcgacggcgagaaccccttcaccatctgcccccaccacctgggcaagaacctggaaaagctgttcaaggcctgggccgacgaaaaaaatcagaccgacgaggaggacgccggcgaaaccgccatcaccgactactgcgccgagttcaaggacggcttcgacagagagcccattagaaacctgctgagatacgtgtacgcttccctgagaagcaagtacagcgccaaggagctgatccaagccgccaagtacaatcagcagttcgagagatatcagagacagaaggcccaccccgtgacccccgacaaccaaggctacacctggcccgaggccgtgatcaagcctagcaaggccgagagaaagaacagagagaacagcctggacacaagaatctgggtgagcgtgaagctgctgcaaaaggacggcacctgggagaagcaccacatccccttctacaacctgagattcttcgaagaggtgtacgccgctgcctccatcgccgaggacatgcctagccccaccttcagaaacagcagattcggcttcaagctgcccaagctgctgaacacccacgccaagatcaacaagaaaaagaaggacggcaagaaggacaagaaggccgtgaacgccgccaagagagaggctagaatctacctcgctgccaaggagggcagactgcccgccccctccgtgcccctggacaagctgagcgccgtgatcgctagagtgaacggcaagttccaagtgaccgtgcccgtgaagttcaaggtgaacaagcagcccaagtaccccctgcctcagctggggcagatcatcctgggctacgatcagaacctgaccgccaaccacgcctgggccctgttcgaggtggtgaccgaggacacccccggcgcccacttctgcagaaatcagtacctgagagtgctcaagaccggccaagtgagaagcatcaccaagaccaaggacggccaagagatcgatcagctgagctacgacggcctggagtacaaggagtacggcgagtggagaaagcaagccaagaagttcgcctcccaatggtacatcaccaccaaggtgagaaagggcaaggaaaccaccctgctgagccaacccgccctggagagattcgagagcatcgagaagtacaagcccgccctgtacagattcaacaaggaatacgcccggctgctcctggccgtcatcagaagaaagaccctgaacgagctggaggagatcagacccgagatcttcagactggtggagcaaggcttcggcatctgcagattcggcagcctgagcctgagcagcttcgaggccatcagagccgccaaggccgtgatctacggctacttcagcaccgccctgaagcacgaccacaacacccccatcacagatgacgagagacaagccttcgaccccgagctgttcggcctgctgaacaagctggaaaccctgagacagaacaagggcgaggagaagatcaacagagccgctaacgccctgatcagaatcgccctggagaacaaggccgacttcatcagaggcgagggcgacctgcccaccaccaacaagagcgtcaagaaggggcagaacagcagaagcatggactggctggctagaggcctgttcaacaagatctgtcagctggccgtgatgcacaacatcattcccaccgccgccaacccccacagcacaagccaccaagaccccttcgagcacaacaaggacttcgacgaccctcagcccgccatgaagtgcagatacgctgagttcaagatcgaggacatcggcgacagcgtgctggagagactgggcacctccctgagaaatgccaaggccgggcagaccggcgcctactattacaagggcgcccaagacttcctggcccactacggcctgcaagacatcgaggaagagctgaagaagtgcagaagaggcagaaaggccaacatgccctgctgggagctgcagaagagactgattcagaagctgggcagcaaggacgccgtggtgtacatccccatgagaggcggccggatctacctggctacacacagcgtgaccaccggcgccaagcccatcatcttcaacggcgaagaggtgtgggtgagcaacgccgacgagatcgccgccgtgaacatcggcctgaccatcatccccacaagcaagaaggacgaggagcaccccgacagagagaacgacgacaaggccatcagaaagagcgtcagacggcctagaaagagcggcgagagaagaagcggcgacaagatgcccgccaccgctagaaagaccacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaacggctcggagatcatcattgcggcgtcaatgggagacgcattacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 698 - DNA, 8956 bp, synthetic construct
  source 1..8956: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccatgccggcagctaagaaaaagaaactggatggcagcgtcgacatcttcgtggtgctgagcgtgaatcagttcagatgcaaccccaacgccttcagacaagcctccatgaaaaccgacgacgagaagaaggacaccgccatcaagagctacacaagctggctgctgcccaacgacagaaaggagaaggacatggtgcggagcttcctggccctggaccaaggcagcagattctttttcgacctgatgcaagcctggtacggcggcctcacccccgacatcatcaagagcgtgaccaaggagcgggacctggtggacctgtggtgcgccatctactggttcagacccatgaagaaggagctggcctcccaccccatcgacagagtggacctggccaagacctttcagagatactacggcggccccgcctccgacgtggtgcaagagtacctgagcgcctccatcggcgaggactgctgctggaacgactgcagacagaagtaccaagagttctgcaagaacctcggggtggacttcacccccgacctcaagaccctggtgagagagaagctgatcgccgtggccctgaacgacggcagccaagccgccatcagcaacctgttcggcaacggcggcaaggaggacagaaacgtgaaggtgaacatctgcaagaagatcctgattgctctggagaaggccggcaaatccgtgaagtacgtgcgggacgtgcagagcatcatcctgaagtgcgccaacgccaaggacagagaggacttcaacagaatctacgccgacaagaacggcagacccggcaccctgctcctgctcctggacagaaagggcgagaacacctacgacgccgaggtgctgaagagatacctgagaaagaccatcaagagcaagaacgcccccctggtgtggaactacaatcagaagctgttcgagtacatcgagagaggcatccggaatctgctgagaagaaccatcaactacgacgcctgggcctggggcgagatgtggagagtgggccacacccccctgcagaccaaggccacaagaaactaccacttcgcccaagagcagctgaagagacacagcgacatcgccgccgcttcccaaagcagccacgccatcgccgtcgctctgaacgagttcttcgagagccccttcttcgacggcgagaaccccttcaccatctgcccccaccacctgggcaagaacctggaaaagctgttcaaggcctgggccgacgaaaaaaatcagaccgacgaggaggacgccggcgaaaccgccatcaccgactactgcgccgagttcaaggacggcttcgacagagagcccattagaaacctgctgagatacgtgtacgcttccctgagaagcaagtacagcgccaaggagctgatccaagccgccaagtacaatcagcagttcgagagatatcagagacagaaggcccaccccgtgacccccgacaaccaaggctacacctggcccgaggccgtgatcaagcctagcaaggccgagagaaagaacagagagaacagcctggacacaagaatctgggtgagcgtgaagctgctgcaaaaggacggcacctgggagaagcaccacatccccttctacaacctgagattcttcgaagaggtgtacgccgctgcctccatcgccgaggacatgcctagccccaccttcagaaacagcagattcggcttcaagctgcccaagctgctgaacacccacgccaagatcaacaagaaaaagaaggacggcaagaaggacaagaaggccgtgaacgccgccaagagagaggctagaatctacctcgctgccaaggagggcagactgcccgccccctccgtgcccctggacaagctgagcgccgtgatcgctagagtgaacggcaagttccaagtgaccgtgcccgtgaagttcaaggtgaacaagcagcccaagtaccccctgcctcagctggggcagatcatcctgggctacgatcagaacctgaccgccaaccacgcctgggccctgttcgaggtggtgaccgaggacacccccggcgcccacttctgcagaaatcagtacctgagagtgctcaagaccggccaagtgagaagcatcaccaagaccaaggacggccaagagatcgatcagctgagctacgacggcctggagtacaaggagtacggcgagtggagaaagcaagccaagaagttcgcctcccaatggtacatcaccaccaaggtgagaaagggcaaggaaaccaccctgctgagccaacccgccctggagagattcgagagcatcgagaagtacaagcccgccctgtacagattcaacaaggaatacgcccggctgctcctggccgtcatcagaagaaagaccctgaacgagctggaggagatcagacccgagatcttcagactggtggagcaaggcttcggcatctgcagattcggcagcctgagcctgagcagcttcgaggccatcagagccgccaaggccgtgatctacggctacttcagcaccgccctgaagcacgaccacaacacccccatcacagatgacgagagacaagccttcgaccccgagctgttcggcctgctgaacaagctggaaaccctgagacagaacaagggcgaggagaagatcaacagagccgctaacgccctgatcagaatcgccctggagaacaaggccgacttcatcagaggcgagggcgacctgcccaccaccaacaagagcgtcaagaaggggcagaacagcagaagcatggactggctggctagaggcctgttcaacaagatctgtcagctggccgtgatgcacaacatcattcccaccgccgccaacccccacagcacaagccaccaagaccccttcgagcacaacaaggacttcgacgaccctcagcccgccatgaagtgcagatacgctgagttcaagatcgaggacatcggcgacagcgtgctggagagactgggcacctccctgagaaatgccaaggccgggcagaccggcgcctactattacaagggcgcccaagacttcctggcccactacggcctgcaagacatcgaggaagagctgaagaagtgcagaagaggcagaaaggccaacatgccctgctgggagctgcagaagagactgattcagaagctgggcagcaaggacgccgtggtgtacatccccatgagaggcggccggatctacctggctacacacagcgtgaccaccggcgccaagcccatcatcttcaacggcgaagaggtgtgggtgagcaacgccgacgagatcgccgccgtgaacatcggcctgaccatcatccccacaagcaagaaggacgaggagcaccccgacagagagaacgacgacaaggccatcagaaagagcgtcagacggcctagaaagagcggcgagagaagaagcggcgacaagatgcccgccaccgctagaaagaccacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 699 - DNA, 51 bp, synthetic construct
  source 1..51: mol_type = other DNA
tggacagcaaatgggtcgcggatccatgccggcagctaagaaaaagaaact

SEQ ID NO: 700 - DNA, 55 bp, synthetic construct
  source 1..55: mol_type = other DNA
tctcagtggtggtggtggtggtgctcgagtcaagcgtaatctggaacatcgtatg

SEQ ID NO: 701 - DNA, 273 bp, synthetic construct
  source 1..273: mol_type = other DNA
ggagttcagacgtgtgctcttccgatctcagcacaaaaggaaactcaccctaactgtaaagtaattgtgtgttttgagactataaatatgcatgcgagaaaagccttgtttgccaccatggaacggctcggagatcatcattgcgnnnnnnngtgagcaagggcgaggagctgttcaccggggtggtgcccatcctggtcgagctggacggcgacgtaaacggccacaagttcagcgtgtccggcagatcggaagagcacacgtctgaactcc

SEQ ID NO: 702 - RNA, 46 nt, synthetic construct
  source 1..46: mol_type = other RNA
gtaatgcgtctcccattgacgcgtgagcaagggcgaggagctgttc

SEQ ID NO: 703 - RNA, 48 nt, synthetic construct
  source 1..48: mol_type = other RNA
gtaatgcgtctcccattgacgccgcaatgatgatctccgagccgttcc

SEQ ID NO: 704 - RNA, 22 nt, synthetic construct
  source 1..22: mol_type = other RNA
gtaatgcgtctcccattgacgc

SEQ ID NO: 705 - DNA, 59 bp, synthetic construct
  source 1..59: mol_type = other DNA
gttgacatgctggattgagacttcctacactctttccctacacgacgctcttccgatct

SEQ ID NO: 706 - DNA, 58 bp, synthetic construct
  source 1..58: mol_type = other DNA
gatcggaagagcgtcgtgtagggaaagagtgtaggaagtctcaatccagcatgtcaac

SEQ ID NO: 707 - DNA, 19 bp, synthetic construct
  source 1..19: mol_type = other DNA
ggagttcagacgtgtgctc

SEQ ID NO: 708 - DNA, 24 bp, synthetic construct
  source 1..24: mol_type = other DNA
gttgacatgctggattgagacttc

SEQ ID NO: 709 - DNA, 58 bp, synthetic construct
  source 1..58: mol_type = other DNA
aatgatacggcgaccaccgagatctacactatagcctacactctttccctacacgacg

SEQ ID NO: 710 - DNA, 57 bp, synthetic construct
  source 1..57: mol_type = other DNA
caagcagaagacggcatacgagatcgagtaatgtgactggagttcagacgtgtgctc

SEQ ID NO: 711 - DNA, 8057 bp, synthetic construct
  source 1..8057: mol_type = other DNA
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattgggatccctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatgcatgcgagaaaagccttgtttgccaccatggaacggctcggagatatccagcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaatctgtctagaagaattcctcgaggcggccgctaaatagttgaatttaaatagatctccagcacagtggtaaatagttgattcgaacaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgcccatgggcatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcg

SEQ ID NO: 712 - DNA, 8131 bp, synthetic construct
  source 1..8131: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 713 - DNA, 8231 bp, synthetic construct
  source 1..8231: mol_type = other DNA
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagctagcgaattgggatccctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatgcatgcgagaaaagccttgtttgccaccatggaacggctcggagatcatcattgcgnnnnnnnaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgaggcggccgctaaatagttgaatttaaatagatctccagcacagtggtaaatagttgattcgaacaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgcccatgggcatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacaattcgagctcgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcg

SEQ ID NO: 714 - DNA, 68 bp, synthetic construct
  source 1..68: mol_type = other DNA
tgtttgccaccatggaacggctcggagatcatcattgcgnnnnnnnaccgagtacaagcccacggtgc

SEQ ID NO: 715 - DNA, 52 bp, synthetic construct
  source 1..52: mol_type = other DNA
taaattcaactatttagcggccgcctcgagtcaggcaccgggcttgcgggtc

SEQ ID NO: 716 - DNA, 2322 bp, synthetic construct
  source 1..2322: mol_type = other DNA
atcataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccccgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttggtacatctcaaataaaacgaaaggctcagtcgaaagactgggcctttcgttttatctgttgtttgtcggtgaacgctctcgttaactagctatactgatttcgtcagactcacagtcaaacatgccggtcagttggcctggtgatggcgggatcgttgtatatttcttgacaccttttcggcaccgccctaaaattctgcgtcctcataatatatgaggcgatttattacgtgtttacgaagcaaaagctaaaaccaggagctatttaatgccggcagctaagaaaaagaaactggatggcagcgtcgacagtcttcgtttcaaacgaatggccaccatgtagaagactgaattcggtaccttgacagctagctcagtcctaggtataatactagtgtttaaaccgtacgtcgacgctagcataaccccttggggcctctaaacgggtcttgaggggttttttggat

SEQ ID NO: 717 - DNA, 223 bp, synthetic construct
  source 1..223: mol_type = other DNA
tcgagtacccatacgatgttccagattacgcttgagaattcggtaccttgacagctagctcagtcctaggtataatactagtgtaatgcgtctcccattgacgccgcaatgatgatctccgagccgttcggccggcatggtcccagcctcctcgctggcgccggctgggcaacatgcttcggcatggcgaatgggaccgtacgtcgacgctagcataacccct

SEQ ID NO: 718 - DNA, 36 bp, synthetic construct
  source 1..36: mol_type = other DNA
atttaatgccggcagctaagaaaaagaaactggatg

SEQ ID NO: 719 - DNA, 5969 bp, synthetic construct
  source 1..5969: mol_type = other DNA
atcataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccccgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttggtacatctcaaataaaacgaaaggctcagtcgaaagactgggcctttcgttttatctgttgtttgtcggtgaacgctctcgttaactagctatactgatttcgtcagactcacagtcaaacatgccggtcagttggcctggtgatggcgggatcgttgtatatttcttgacaccttttcggcaccgccctaaaattctgcgtcctcataatatatgaggcgatttattacgtgtttacgaagcaaaagctaaaaccaggagctatttaatgccggcagctaagaaaaagaaactggatggcagcgtcgacatcttcgtggtgctgagcgtgaatcagttcagatgcaaccccaacgccttcagacaagcctccatgaaaaccgacgacgagaagaaggacaccgccatcaagagctacacaagctggctgctgcccaacgacagaaaggagaaggacatggtgcggagcttcctggccctggaccaaggcagcagattctttttcgacctgatgcaagcctggtacggcggcctcacccccgacatcatcaagagcgtgaccaaggagcgggacctggtggacctgtggtgcgccatctactggttcagacccatgaagaaggagctggcctcccaccccatcgacagagtggacctggccaagacctttcagagatactacggcggccccgcctccgacgtggtgcaagagtacctgagcgcctccatcggcgaggactgctgctggaacgactgcagacagaagtaccaagagttctgcaagaacctcggggtggacttcacccccgacctcaagaccctggtgagagagaagctgatcgccgtggccctgaacgacggcagccaagccgccatcagcaacctgttcggcaacggcggcaaggaggacagaaacgtgaaggtgaacatctgcaagaagatcctgattgctctggagaaggccggcaaatccgtgaagtacgtgcgggacgtgcagagcatcatcctgaagtgcgccaacgccaaggacagagaggacttcaacagaatctacgccgacaagaacggcagacccggcaccctgctcctgctcctggacagaaagggcgagaacacctacgacgccgaggtgctgaagagatacctgagaaagaccatcaagagcaagaacgcccccctggtgtggaactacaatcagaagctgttcgagtacatcgagagaggcatccggaatctgctgagaagaaccatcaactacgacgcctgggcctggggcgagatgtggagagtgggccacacccccctgcagaccaaggccacaagaaactaccacttcgcccaagagcagctgaagagacacagcgacatcgccgccgcttcccaaagcagccacgccatcgccgtcgctctgaacgagttcttcgagagccccttcttcgacggcgagaaccccttcaccatctgcccccaccacctgggcaagaacctggaaaagctgttcaaggcctgggccgacgaaaaaaatcagaccgacgaggaggacgccggcgaaaccgccatcaccgactactgcgccgagttcaaggacggcttcgacagagagcccattagaaacctgctgagatacgtgtacgcttccctgagaagcaagtacagcgccaaggagctgatccaagccgccaagtacaatcagcagttcgagagatatcagagacagaaggcccaccccgtgacccccgacaaccaaggctacacctggcccgaggccgtgatcaagcctagcaaggccgagagaaagaacagagagaacagcctggacacaagaatctgggtgagcgtgaagctgctgcaaaaggacggcacctgggagaagcaccacatccccttctacaacctgagattcttcgaagaggtgtacgccgctgcctccatcgccgaggacatgcctagccccaccttcagaaacagcagattcggcttcaagctgcccaagctgctgaacacccacgccaagatcaacaagaaaaagaaggacggcaagaaggacaagaaggccgtgaacgccgccaagagagaggctagaatctacctcgctgccaaggagggcagactgcccgccccctccgtgcccctggacaagctgagcgccgtgatcgctagagtgaacggcaagttccaagtgaccgtgcccgtgaagttcaaggtgaacaagcagcccaagtaccccctgcctcagctggggcagatcatcctgggctacgatcagaacctgaccgccaaccacgcctgggccctgttcgaggtggtgaccgaggacacccccggcgcccacttctgcagaaatcagtacctgagagtgctcaagaccggccaagtgagaagcatcaccaagaccaaggacggccaagagatcgatcagctgagctacgacggcctggagtacaaggagtacggcgagtggagaaagcaagccaagaagttcgcctcccaatggtacatcaccaccaaggtgagaaagggcaaggaaaccaccctgctgagccaacccgccctggagagattcgagagcatcgagaagtacaagcccgccctgtacagattcaacaaggaatacgcccggctgctcctggccgtcatcagaagaaagaccctgaacgagctggaggagatcagacccgagatcttcagactggtggagcaaggcttcggcatctgcagattcggcagcctgagcctgagcagcttcgaggccatcagagccgccaaggccgtgatctacggctacttcagcaccgccctgaagcacgaccacaacacccccatcacagatgacgagagacaagccttcgaccccgagctgttcggcctgctgaacaagctggaaaccctgagacagaacaagggcgaggagaagatcaacagagccgctaacgccctgatcagaatcgccctggagaacaaggccgacttcatcagaggcgagggcgacctgcccaccaccaacaagagcgtcaagaaggggcagaacagcagaagcatggactggctggctagaggcctgttcaacaagatctgtcagctggccgtgatgcacaacatcattcccaccgccgccaacccccacagcacaagccaccaagaccccttcgagcacaacaaggacttcgacgaccctcagcccgccatgaagtgcagatacgctgagttcaagatcgaggacatcggcgacagcgtgctggagagactgggcacctccctgagaaatgccaaggccgggcagaccggcgcctactattacaagggcgcccaagacttcctggcccactacggcctgcaagacatcgaggaagagctgaagaagtgcagaagaggcagaaaggccaacatgccctgctgggagctgcagaagagactgattcagaagctgggcagcaaggacgccgtggtgtacatccccatgagaggcggccggatctacctggctacacacagcgtgaccaccggcgccaagcccatcatcttcaacggcgaagaggtgtgggtgagcaacgccgacgagatcgccgccgtgaacatcggcctgaccatcatccccacaagcaagaaggacgaggagcaccccgacagagagaacgacgacaaggccatcagaaagagcgtcagacggcctagaaagagcggcgagagaagaagcggcgacaagatgcccgccaccgctagaaagaccacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattcggtaccttgacagctagctcagtcctaggtataatactagtgtaatgcgtctcccattgacgccgcaatgatgatctccgagccgttcggccggcatggtcccagcctcctcgctggcgccggctgggcaacatgcttcggcatggcgaatgggaccgtacgtcgacgctagcataaccccttggggcctctaaacgggtcttgaggggttttttggat

SEQ ID NO: 720 - DNA, 28 bp, synthetic construct
  source 1..28: mol_type = other DNA
cagcacaaaaggaaactcaccctaactg

SEQ ID NO: 721 - DNA, 20 bp, synthetic construct
  source 1..20: mol_type = other DNA
tgtggcgcgtggcggggtag

SEQ ID NO: 722 - DNA, 23 bp, synthetic construct
  source 1..23: mol_type = other DNA
catgagcatgcagaggtgagtat

SEQ ID NO: 723 - DNA, 38 bp, synthetic construct
  source 1..38: mol_type = other DNA
cattgacgccatgagcatgcagaggtgagtattttttg

SEQ ID NO: 724 - DNA, 38 bp, synthetic construct
  source 1..38: mol_type = other DNA
gtaccaaaaaatactcacctctgcatgctcatggcgtc

SEQ ID NO: 725 - DNA, 8602 bp, synthetic construct
  source 1..8602: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccatgccggcagctaagaaaaagaaactggatggcagcgtcgacatcttccccacacagagaagaaacaccatgagcggcaacgtggtgagaagctacaagagcgtgctcagacccaacatcagaaagagagagctgctggacgccaccttcaactggttcgacagagcctacaagaagttcttcgacgtgttcgtgtgcctgtacggcggcgtggagcacgacaccgtgcacagcgccctgctgaaggaaaccaccgaccccgacctggtgtgcgccaccatgtggttcagagtggtgcccaagagcagctgcgacggcatcagcgcccaagagatgatcagaagattcggcgtgtacgccggccacgagcctagcgccgtggccatgagctacctgaccggcaacttcgacgacaagaagaaccactggatcgactgcagagagaagttcgtggccctggctagagacatggtggtgaagcccgagagcctgagcatcgacatcaagagcatgatcgagcacaagatcctgcccatctgcagccaagacaactggcaagcctggaacgccatcagccaactgttcggcgagggcaagaaggagaacaaggccgagaaggctcagatcttcgtggacgtggtgagcatgctgagcaacaacagcgtgcagacctgggaggactacaaagaggtgatcagcaaggccaccggctgcagaaccgccggcgagatcaacagcaagttcggcggcagacccggcatcctgagcgtggacttcagcaaggacgacaccggcagcctgcccaaggacttcatcgagagaagaatcaaggacctgagccaaaaggccaaggagaagagcgccgcctacgagctgcccaacagaatgagactgagagagctgatcgctagcgtgatcggcccctacagactggaaacctggagcagcgtggctcagagagcctgcggcgacatcagaagcaagaacagcaacaacctgctgtacgctagcgagagattcaacagaaccaaggagatcgaggagatcctgagcgccaagggcgacgtggctagagcccaaaacatcctagggcagttcagaagcggcgagttcaacgagttcgccgtggagaagagacacctgggcaacctggagagcctgtacaacatctggagcaagagcgacatggacaccggcatcgaggagtacagcagcatccacaaggacgagtacgaccgggaccccatcgtggacctgtacagacacatctacccccacagagaggtgatcagcgccaagaacttcatcgacgccgccgtgctgaacaagctgctgagactgaacgctagcagacacgtgcaccccaccgtgttcggcaagaccgtgatcagcttcagccccaagagcagcgcctacggcagaatcaccccccctagcgagatcgtgagaggcagacccgccggcagccacggcatgatctggctgaccatggagctgttcgacgacggcaagtggatcgagcaccacctgcccttccacaacagcagatactacgaagaggtgtactgctaccaagagggcctgcccgtgaaggacgagcctagaagccccatgttcggctacagagtgggcaacgccatcgccgacacaagcaagatcgacaacagacggagaaaggctagcaagcagttcctgagagctcagcagaacatcacccacaacgtgagcttcgacgagaacaccgccttcagcgtggtgagaaacggcgaggacttcagcatcaccatcagcagcagaatcaaggctagcaatgctaagagcctgatgaccatcggcgacagaatcatgggcatcgatcagaatcagaccgccgccaacacctacagcatctgggaggtggtgggcgacggcgacgccggcagcattcagcacggcaacctgagcctgaagagaatcgaggacggccacatcacaagcatcatcagaggcagaggcggcaacttcgatcagctgaactacggcggcctgagctacgccaagttcagcgagtggagaagcgctagactgggcttcatcagcagcctgaacgccgagctggctagcaagatgaactgggactggtgctgcctgtacgagtggaacgctagatacgctagcgccctgagaaagatcatctacagccacaagggcatcgacatcgagagagtgttcagaagagagatcgaggacttcgtggagggcgtgctgagaatcggcagcctgagcagcgacgccctgcagtgcctgaccaacgccaagagcctgatcagcagctacttcttcctgaacggcaagaaggagcccgaggagcagagaaagttcgaccaaggcctgttcgacctgagcgccaagattgacaagaagagagtgaacaagagacagcaaaagaccaagagaatcatgagcagcatcatgcagatcgctagagacagaggcgtgagcttcatcgtggtggagggcaagctgagcaccgccaccaaggacaacaagagcaaggccaatcagaaggccatcgactggtgcgctagagccgtggtggagaacctggagcacagctgcagcctggtgggcatcaagctggtggccatcgaccccatgaacacaagccacctggaccccttcgtgtacgtgctcaagacaagcctgggcaaggaggctagattcgctagcgtggtgcccgccaagatcaacgctagacacatgaagaacttcaagagctggagcagcctgctggccggcaacggcaagatcaaaaagaccaccgacgccatctacgtgggcgccttccaagacttctgcagagagtacggcttcgtggccgaggacatcagcaagatgagccaaagcgagatccaagacaagctgagcagccatcagttcgtgctggtgcctcagagaggcggcagagtgtacatgagcacccaccccgtgagcagcgaggccaagaagatcgtgtacgccggcagagagagatggtacaacaacgccgacgtggtggccgccgtgaacatcaccctgagaggctgcgagagactgagcgctagcagaaagaccgctagcacaagaacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 726 - RNA, 60 nt, synthetic construct
  source 1..60: mol_type = other RNA
atcgcaacatctcagaaacccgtcctaagttgacgggtgagcaagggcgaggagctgttc

SEQ ID NO: 727 - RNA, 62 nt, synthetic construct
  source 1..62: mol_type = other RNA
atcgcaacatctcagaaacccgtcctaagttgacggcgcaatgatgatctccgagccgttcc

SEQ ID NO: 728 - protein, 1021 aa, synthetic construct
IFPTQRRNTMSGNVVRSYKSVLRPNIRKRELLDATFNWFDRAYKKFFDVFVCLYGGVEHDTVHSALLKETTDPDLVCATMWFRVVPKSSCDGISAQEMIRRFGVYAGHEPSAVAMSYLTGNFDDKKNHWIDCREKFVALARDMVVKPESLSIDIKSMIEHKILPICSQDNWQAWNAISQLFGEGKKENKAEKAQIFVDVVSMLSNNSVQTWEDYKEVISKATGCRTAGEINSKFGGRPGILSVDFSKDDTGSLPKDFIERRIKDLSQKAKEKSAAYELPNRMRLRELIASVIGPYRLETWSSVAQRACGDIRSKNSNNLLYASERFNRTKEIEEILSAKGDVARAQSILGQFRSGEFNEFAVEKRHLGNLESLYNIWSKSDMDTGIEEYSSIHKDEYDRDPIVDLYRHIYPHREVISAKNFIDAAVLNKLLRLNASRHVHPTVFGKTVISFSPKSSAYGRITPPSEIVRGRPAGSHGMIWLTMELFDDGKWIEHHLPFHNSRYYEEVYCYQEGLPVKDEPRSPMFGYRVGNAIADTSKIDNRRRKASKQFLRAQQNITHNVSFDENTAFSVVRNGEDFSITISSRIKASNAKSLMTIGDRIMGIDQNQTAANTYSIWEVVGDGDAGSIQHGNLSLKRIEDGHITSIIRGRGGNFDQLNYGGLSYAKFSEWRSARLGFISSLNAELASKMNWDWCCLYEWNARYASALRKIIYSHKGIDIERVFRREIEDFVEGVLRIGSLSSDALQCLTNAKSLISSYFFLNGKKEPEEQRKFDQGLFDLSAKIDKKRVNKRQQKTKRIMSSIMQIARDRGVSFIVVEGKLSTATKDNKSKANQKAIDWCARAVVENLEHSCSLVGIKLVAIDPMNTSHLDPFVYVLKTSLGKEARFASVVPAKINARHMKNFKSWSSLLAGNGKIKKTTDAIYVGAFQDFCREYGFVAEDISKMSQSEIQDKLSSHQFVLVPQRGGRVYMSTHPVSSEAKKIVYAGRERWYNNADVVAAVNITLRGCERLSASRKTASTR

SEQ ID NO: 729 - DNA, 5629 bp, synthetic construct
  source 1..5629: mol_type = other DNA
atcataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccccgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttggtacatctcaaataaaacgaaaggctcagtcgaaagactgggcctttcgttttatctgttgtttgtcggtgaacgctctcgttaactagctatactgatttcgtcagactcacagtcaaacatgccggtcagttggcctggtgatggcgggatcgttgtatatttcttgacaccttttcggcaccgccctaaaattctgcgtcctcataatatatgaggcgatttattacgtgtttacgaagcaaaagctaaaaccaggagctatttaatgccggcagctaagaaaaagaaactggatggcagcgtcgacatcttccccacacagagaagaaacaccatgagcggcaacgtggtgagaagctacaagagcgtgctcagacccaacatcagaaagagagagctgctggacgccaccttcaactggttcgacagagcctacaagaagttcttcgacgtgttcgtgtgcctgtacggcggcgtggagcacgacaccgtgcacagcgccctgctgaaggaaaccaccgaccccgacctggtgtgcgccaccatgtggttcagagtggtgcccaagagcagctgcgacggcatcagcgcccaagagatgatcagaagattcggcgtgtacgccggccacgagcctagcgccgtggccatgagctacctgaccggcaacttcgacgacaagaagaaccactggatcgactgcagagagaagttcgtggccctggctagagacatggtggtgaagcccgagagcctgagcatcgacatcaagagcatgatcgagcacaagatcctgcccatctgcagccaagacaactggcaagcctggaacgccatcagccaactgttcggcgagggcaagaaggagaacaaggccgagaaggctcagatcttcgtggacgtggtgagcatgctgagcaacaacagcgtgcagacctgggaggactacaaagaggtgatcagcaaggccaccggctgcagaaccgccggcgagatcaacagcaagttcggcggcagacccggcatcctgagcgtggacttcagcaaggacgacaccggcagcctgcccaaggacttcatcgagagaagaatcaaggacctgagccaaaaggccaaggagaagagcgccgcctacgagctgcccaacagaatgagactgagagagctgatcgctagcgtgatcggcccctacagactggaaacctggagcagcgtggctcagagagcctgcggcgacatcagaagcaagaacagcaacaacctgctgtacgctagcgagagattcaacagaaccaaggagatcgaggagatcctgagcgccaagggcgacgtggctagagcccaaagcatcctagggcagttcagaagcggcgagttcaacgagttcgccgtggagaagagacacctgggcaacctggagagcctgtacaacatctggagcaagagcgacatggacaccggcatcgaggagtacagcagcatccacaaggacgagtacgaccgggaccccatcgtggacctgtacagacacatctacccccacagagaggtgatcagcgccaagaacttcatcgacgccgccgtgctgaacaagctgctgagactgaacgctagcagacacgtgcaccccaccgtgttcggcaagaccgtgatcagcttcagccccaagagcagcgcctacggcagaatcaccccccctagcgagatcgtgagaggcagacccgccggcagccacggcatgatctggctgaccatggagctgttcgacgacggcaagtggatcgagcaccacctgcccttccacaacagcagatactacgaagaggtgtactgctaccaagagggcctgcccgtgaaggacgagcctagaagccccatgttcggctacagagtgggcaacgccatcgccgacacaagcaagatcgacaacagacggagaaaggctagcaagcagttcctgagagctcagcagaacatcacccacaacgtgagcttcgacgagaacaccgccttcagcgtggtgagaaacggcgaggacttcagcatcaccatcagcagcagaatcaaggctagcaatgctaagagcctgatgaccatcggcgacagaatcatgggcatcgatcagaatcagaccgccgccaacacctacagcatctgggaggtggtgggcgacggcgacgccggcagcattcagcacggcaacctgagcctgaagagaatcgaggacggccacatcacaagcatcatcagaggcagaggcggcaacttcgatcagctgaactacggcggcctgagctacgccaagttcagcgagtggagaagcgctagactgggcttcatcagcagcctgaacgccgagctggctagcaagatgaactgggactggtgctgcctgtacgagtggaacgctagatacgctagcgccctgagaaagatcatctacagccacaagggcatcgacatcgagagagtgttcagaagagagatcgaggacttcgtggagggcgtgctgagaatcggcagcctgagcagcgacgccctgcagtgcctgaccaacgccaagagcctgatcagcagctacttcttcctgaacggcaagaaggagcccgaggagcagagaaagttcgaccaaggcctgttcgacctgagcgccaagattgacaagaagagagtgaacaagagacagcaaaagaccaagagaatcatgagcagcatcatgcagatcgctagagacagaggcgtgagcttcatcgtggtggagggcaagctgagcaccgccaccaaggacaacaagagcaaggccaatcagaaggccatcgactggtgcgctagagccgtggtggagaacctggagcacagctgcagcctggtgggcatcaagctggtggccatcgaccccatgaacacaagccacctggaccccttcgtgtacgtgctcaagacaagcctgggcaaggaggctagattcgctagcgtggtgcccgccaagatcaacgctagacacatgaagaacttcaagagctggagcagcctgctggccggcaacggcaagatcaaaaagaccaccgacgccatctacgtgggcgccttccaagacttctgcagagagtacggcttcgtggccgaggacatcagcaagatgagccaaagcgagatccaagacaagctgagcagccatcagttcgtgctggtgcctcagagaggcggcagagtgtacatgagcacccaccccgtgagcagcgaggccaagaagatcgtgtacgccggcagagagagatggtacaacaacgccgacgtggtggccgccgtgaacatcaccctgagaggctgcgagagactgagcgctagcagaaagaccgctagcacaagaacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattcggtaccttgacagctagctcagtcctaggtataatactagtatcgcaacatctcagaaacccgtcctaagttgacggcgcaatgatgatctccgagccgttcggccggcatggtcccagcctcctcgctggcgccggctgggcaacatgcttcggcatggcgaatgggaccgtacgtcgacgctagcataaccccttggggcctctaaacgggtcttgaggggttttttggat

SEQ ID NO: 730 - DNA, 8134 bp, synthetic construct
  source 1..8134: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgnaangtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaactcgaggcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 731 - DNA, 27 bp, synthetic construct
  source 1..27: mol_type = other DNA
ccaagctggctagcgtttaaacttaag

SEQ ID NO: 732 - DNA, 26 bp, synthetic construct
  source 1..26: mol_type = other DNA
atgatctccgagccgtttttggtacc

SEQ ID NO: 733 - DNA, 31 bp, synthetic construct
  source 1..31: mol_type = other DNA
gctgaagagacacagcagaatcgccgccgct

SEQ ID NO: 734 - DNA, 30 bp, synthetic construct
  source 1..30: mol_type = other DNA
gggaagcggcggcgattctgctgtgtctct

SEQ ID NO: 735 - DNA, 22 bp, synthetic construct
  source 1..22: mol_type = other DNA
ctcacctcgcgacgcaatgatg

SEQ ID NO: 736 - DNA, 26 bp, synthetic construct
  source 1..26: mol_type = other DNA
gcgacgcaatgatgtttttcggtacc

SEQ ID NO: 737 - DNA, 9036 bp, synthetic construct
  source 1..9036: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacatcttccccacacagagaagaaacaccatgagcggcaacgtggtgagaagctacaagagcgtgctcagacccaacatcagaaagagagagctgctggacgccaccttcaactggttcgacagagcctacaagaagttcttcgacgtgttcgtgtgcctgtacggcggcgtggagcacgacaccgtgcacagcgccctgctgaaggaaaccaccgaccccgacctggtgtgcgccaccatgtggttcagagtggtgcccaagagcagctgcgacggcatcagcgcccaagagatgatcagaagattcggcgtgtacgccggccacgagcctagcgccgtggccatgagctacctgaccggcaacttcgacgacaagaagaaccactggatcgactgcagagagaagttcgtggccctggctagagacatggtggtgaagcccgagagcctgagcatcgacatcaagagcatgatcgagcacaagatcctgcccatctgcagccaagacaactggcaagcctggaacgccatcagccaactgttcggcgagggcaagaaggagaacaaggccgagaaggctcagatcttcgtggacgtggtgagcatgctgagcaacaacagcgtgcagacctgggaggactacaaagaggtgatcagcaaggccaccggctgcagaaccgccggcgagatcaacagcaagttcggcggcagacccggcatcctgagcgtggacttcagcaaggacgacaccggcagcctgcccaaggacttcatcgagagaagaatcaaggacctgagccaaaaggccaaggagaagagcgccgcctacgagctgcccaacagaatgagactgagagagctgatcgctagcgtgatcggcccctacagactggaaacctggagcagcgtggctcagagagcctgcggcgacatcagaagcaagaacagcaacaacctgctgtacgctagcgagagattcaacagaaccaaggagatcgaggagatcctgagcgccaagggcgacgtggctagagcccaaaacatcctagggcagttcagaagcggcgagttcaacgagttcgccgtggagaagagacacctgggcaacctggagagcctgtacaacatctggagcaagagcgacatggacaccggcatcgaggagtacagcagcatccacaaggacgagtacgaccgggaccccatcgtggacctgtacagacacatctacccccacagagaggtgatcagcgccaagaacttcatcgacgccgccgtgctgaacaagctgctgagactgaacgctagcagacacgtgcaccccaccgtgttcggcaagaccgtgatcagcttcagccccaagagcagcgcctacggcagaatcaccccccctagcgagatcgtgagaggcagacccgccggcagccacggcatgatctggctgaccatggagctgttcgacgacggcaagtggatcgagcaccacctgcccttccacaacagcagatactacgaagaggtgtactgctaccaagagggcctgcccgtgaaggacgagcctagaagccccatgttcggctacagagtgggcaacgccatcgccgacacaagcaagatcgacaacagacggagaaaggctagcaagcagttcctgagagctcagcagaacatcacccacaacgtgagcttcgacgagaacaccgccttcagcgtggtgagaaacggcgaggacttcagcatcaccatcagcagcagaatcaaggctagcaatgctaagagcctgatgaccatcggcgacagaatcatgggcatcgatcagaatcagaccgccgccaacacctacagcatctgggaggtggtgggcgacggcgacgccggcagcattcagcacggcaacctgagcctgaagagaatcgaggacggccacatcacaagcatcatcagaggcagaggcggcaacttcgatcagctgaactacggcggcctgagctacgccaagttcagcgagtggagaagcgctagactgggcttcatcagcagcctgaacgccgagctggctagcaagatgaactgggactggtgctgcctgtacgagtggaacgctagatacgctagcgccctgagaaagatcatctacagccacaagggcatcgacatcgagagagtgttcagaagagagatcgaggacttcgtggagggcgtgctgagaatcggcagcctgagcagcgacgccctgcagtgcctgaccaacgccaagagcctgatcagcagctacttcttcctgaacggcaagaaggagcccgaggagcagagaaagttcgaccaaggcctgttcgacctgagcgccaagattgacaagaagagagtgaacaagagacagcaaaagaccaagagaatcatgagcagcatcatgcagatcgctagagacagaggcgtgagcttcatcgtggtggagggcaagctgagcaccgccaccaaggacaacaagagcaaggccaatcagaaggccatcgactggtgcgctagagccgtggtggagaacctggagcacagctgcagcctggtgggcatcaagctggtggccatcgaccccatgaacacaagccacctggaccccttcgtgtacgtgctcaagacaagcctgggcaaggaggctagattcgctagcgtggtgcccgccaagatcaacgctagacacatgaagaacttcaagagctggagcagcctgctggccggcaacggcaagatcaaaaagaccaccgacgccatctacgtgggcgccttccaagacttctgcagagagtacggcttcgtggccgaggacatcagcaagatgagccaaagcgagatccaagacaagctgagcagccatcagttcgtgctggtgcctcagagaggcggcagagtgtacatgagcacccaccccgtgagcagcgaggccaagaagatcgtgtacgccggcagagagagatggtacaacaacgccgacgtggtggccgccgtgaacatcaccctgagaggctgcgagagactgagcgctagcagaaagaccgctagcacaagaacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaacggctcggagatcatcattgcgccgtcaacttaggacgggtttctgagatgttgcgatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 738 - protein, 7 aa, synthetic construct
PKKKRKV

SEQ ID NO: 739 - protein, 16 aa, synthetic construct
KRPAATKKAGQAKKKK

SEQ ID NO: 740 - protein, 9 aa, synthetic construct
PAAKRVKLD

SEQ ID NO: 741 - protein, 11 aa, synthetic construct
RQRRNELKRSP

SEQ ID NO: 742 - protein, 38 aa, synthetic construct
NQSSNFGPMKGGNFGGRSSGPYGGGGQYFAKPRNQGGY

SEQ ID NO: 743 - protein, 42 aa, synthetic construct
RMRIZFKNKGKDTAELRRRRVEVSVELRKAKKDEQILKRRNV

SEQ ID NO: 744 - protein, 8 aa, synthetic construct
VSRKRPRP

SEQ ID NO: 745 - protein, 8 aa, synthetic construct
PPKKARED

SEQ ID NO: 746 - protein, 8 aa, synthetic construct
PQPKKKPL

SEQ ID NO: 747 - protein, 12 aa, synthetic construct
SALIKKKKKMAP

SEQ ID NO: 748 - protein, 5 aa, synthetic construct
DRLRR

SEQ ID NO: 749 - protein, 7 aa, synthetic construct
PKQKKRK

SEQ ID NO: 750 - protein, 10 aa, synthetic construct
RKLKKKIKKL

SEQ ID NO: 751 - protein, 10 aa, synthetic construct
REKKKFLKRR

SEQ ID NO: 752 - protein, 20 aa, synthetic construct
KRKGDEVDGVDEVAKKKSKK

SEQ ID NO: 753 - protein, 17 aa, synthetic construct
RKCLQAGMNLEARKTKK

SEQ ID NO: 754 - DNA, 38 bp, synthetic construct
  source 1..38: mol_type = other DNA
gtaccgaaaaacatcattgcgtcgcgaggtgaggcgtc

SEQ ID NO: 755 - DNA, 38 bp, synthetic construct
  source 1..38: mol_type = other DNA
cattgacgcctcacctcgcgacgcaatgatgtttttcg

SEQ ID NO: 756 - DNA, 28 bp, synthetic construct
  source 1..28: mol_type = other DNA
gagttcaagagaggcttcgacagagagc

SEQ ID NO: 757 - DNA, 30 bp, synthetic construct
  source 1..30: mol_type = other DNA
tcgaagcctctcttgaactcggcgcagtag

SEQ ID NO: 758 - DNA, 26 bp, synthetic construct
  source 1..26: mol_type = other DNA
aaccaggagacagaacaagggcgagg

SEQ ID NO: 759 - DNA, 34 bp, synthetic construct
  source 1..34: mol_type = other DNA
ccttgttctgtctcctggtttccagcttgttcag

SEQ ID NO: 760 - protein, 1139 aa, synthetic construct
RFVVLSVNQFRCNPNAFRQASMKTDDEKKDTAIKSYTSWLLPNDRKEKDMVRSFLALDQGSRFFFDLMQAWYGGLTPDIIKSVTKERDLVDLWCAIYWFRPMKKELASHPIDRVDLAKTFQRYYGGPASDVVQEYLSASIGEDCCWNDCRQKYQEFCKNLGVDFTPDLKTLVREKLIAVALNDGSRAAISNLFGNGGKEDRNVKVNICKKILIALEKAGKSVKYVRDVQSIILKCANAKDREDFNRIYADKNGRPGTLLLLLDRKGENTYDAEVLKRYLRKTIKSKNAPLVWNYNQKLFEYIERGIRNLLRRTINYDAWAWGEMWRVGHTPLQTKATRNYHFAQEQLKRHSRIAAASQSSHAIAVALNEFFESPFFDGENPFTICPHHLGKNLEKLFKAWADEKNQTDEEDAGETAITDYCAEFKRGFDREPIRNLLRYVYASLRSKYSAKELIQAAKYNQQFERYQRQKAHPVTPDNQGYTWPEAVIKPSKAERKNRENSLDTRIWVSVKLLQKDGTWEKHHIPFYNLRFFEEVYAAASIAEDMPSPTFRNSRFGFKLPKLLNTHAKINKKKKDGKKDKKAVNAAKREARIYLAAKEGRLPAPSVPLDKLSAVIARVNGKFQVTVPVKFKVNKQPKYPLPQLGQIILGYDQNLTANHAWALFEVVTEDTPGAHFCRNQYLRVLKTGQVRSITKTKDGQEIDQLSYDGLEYKEYGEWRKQAKKFASQWYITTKVRKGKETTLLSQPALERFESIEKYKPALYRFNKEYARLLLAVIRRKTLNELEEIRPEIFRLVEQGFGICRFGSLSLSSFEAIRAAKAVIYGYFSTALKHDHNTPITDDERQARDPELFGLLNKLRTRRQNKGEEKINRAANALIRIALENKADFIRGEGDLPTTNKSVKKGQNSRSMDWLARGLFNKICQLAVMHNIIPTAANPHSTSHQDPFEHNKDFDDPQPAMKCRYAEFKIEDIGDSVLERLGTSLRNAKAGQTGAYYYKGAQDFLAHYGLQDIEEELKKCRRGRKANMPCWELQKRLIQKLGSKDAVVYIPMRGGRIYLATHSVTTGAKPIIFNGEEVWVSNADEIAAVNIGLTIIPTSKKDEEHPDRENDDKAIRKSVRRPRKSGERRSGDKMPATARKT

SEQ ID NO: 761 - DNA, 23 bp, synthetic construct
  source 1..23: mol_type = other DNA
tcctataaggtgtgaaagtctgg

SEQ ID NO: 762 - DNA, 20 bp, synthetic construct
  source 1..20: mol_type = other DNA
cagtaagatttggtgtctat

SEQ ID NO: 763 - DNA, 23 bp, synthetic construct
  source 1..23: mol_type = other DNA
tatatcccttctacaaattcctc

SEQ ID NO: 764 - DNA, 24 bp, synthetic construct
  source 1..24: mol_type = other DNA
gtgtctgaggctggccctacggtg

SEQ ID NO: 765 - DNA, 23 bp, synthetic construct
  source 1..23: mol_type = other DNA
agaaaggctgctgatgacacctg

SEQ ID NO: 766 - DNA, 24 bp, synthetic construct
  source 1..24: mol_type = other DNA
gcatctccccattccatgagcatg

SEQ ID NO: 767 - DNA, 22 bp, synthetic construct
  source 1..22: mol_type = other DNA
acagccaacgactccggccccg

SEQ ID NO: 768 - DNA, 23 bp, synthetic construct
  source 1..23: mol_type = other DNA
tagaagggatatacaaagtggaa

SEQ ID NO: 769 - DNA, 24 bp, synthetic construct
  source 1..24: mol_type = other DNA
tagatgctgtccgaggcagtcctg

SEQ ID NO: 770 - DNA, 23 bp, synthetic construct
  source 1..23: mol_type = other DNA
ggtgtctatttccactttgtata

SEQ ID NO: 771 - DNA, 24 bp, synthetic construct
  source 1..24: mol_type = other DNA
ctgaacacatgcacggccacattg

SEQ ID NO: 772 - DNA, 23 bp, synthetic construct
  source 1..23: mol_type = other DNA
cccagaggcaaatggctcccagg

SEQ ID NO: 773 - DNA, 23 bp, synthetic construct
  source 1..23: mol_type = other DNA
ctcctcagttgtgagcccatgca

SEQ ID NO: 774 - DNA, 23 bp, synthetic construct
  source 1..23: mol_type = other DNA
gccagccttgccttgaccaatag

SEQ ID NO: 775 - DNA, 23 bp, synthetic construct
  source 1..23: mol_type = other DNA
accaatagtcttagagtatccag

SEQ ID NO: 776 - DNA, 23 bp, synthetic construct
  source 1..23: mol_type = other DNA
ccttgtcaaggctattggtcaag

SEQ ID NO: 777 - DNA, 23 bp, synthetic construct
  source 1..23: mol_type = other DNA
ccttgaccaatagccttgacaag

SEQ ID NO: 778 - DNA, 23 bp, synthetic construct
  source 1..23: mol_type = other DNA
accaatagccttgacaaggcaaa

SEQ ID NO: 779 - DNA, 23 bp, synthetic construct
  source 1..23: mol_type = other DNA
gtcaaggcaaggctggccaaccc

SEQ ID NO: 780 - DNA, 21 bp, synthetic construct
  source 1..21: mol_type = other DNA
gtcaagtttgcctgtcaaggc

SEQ ID NO: 781 - DNA, 23 bp, synthetic construct
  source 1..23: mol_type = other DNA
tcaaggctattggtcaaggcaag

SEQ ID NO: 782 - DNA, 24 bp, synthetic construct
  source 1..24: mol_type = other DNA
acaaggcaaacttgaccaatagtc

SEQ ID NO: 783 - RNA, 45 nt, synthetic construct
  source 1..45: mol_type = other RNA
gtaatgcgtctcccattgacgccatgagcatgcagaggtgagtat

SEQ ID NO: 784 - RNA, 45 nt, synthetic construct
  source 1..45: mol_type = other RNA
gtaatgcgtctcccattgacgctcctataaggtgtgaaagtctgg

SEQ ID NO: 785 - RNA, 42 nt, synthetic construct
  source 1..42: mol_type = other RNA
gtaatgcgtctcccattgacgccagtaagatttggtgtctat

SEQ ID NO: 786 - RNA, 45 nt, synthetic construct
  source 1..45: mol_type = other RNA
gtaatgcgtctcccattgacgctatatcccttctacaaattcctc

SEQ ID NO: 787 - RNA, 46 nt, synthetic construct
  source 1..46: mol_type = other RNA
gtaatgcgtctcccattgacgcgtgtctgaggctggccctacggtg

SEQ ID NO: 788 - RNA, 45 nt, synthetic construct
  source 1..45: mol_type = other RNA
gtaatgcgtctcccattgacgcagaaaggctgctgatgacacctg

SEQ ID NO: 789 - RNA, 46 nt, synthetic construct
  source 1..46: mol_type = other RNA
gtaatgcgtctcccattgacgcgcatctccccattccatgagcatg

SEQ ID NO: 790 - RNA, 45 nt, synthetic construct
  source 1..45: mol_type = other RNA
gtaatgcgtctcccattgacgcacagccaacgactccggcccccg

SEQ ID NO: 791 - RNA, 45 nt, synthetic construct
  source 1..45: mol_type = other RNA
gtaatgcgtctcccattgacgctagaagggatatacaaagtggaa

SEQ ID NO: 792 - RNA, 46 nt, synthetic construct
  source 1..46: mol_type = other RNA
gtaatgcgtctcccattgacgctagatgctgtccgaggcagtcctg

SEQ ID NO: 793 - RNA, 45 nt, synthetic construct
  source 1..45: mol_type = other RNA
gtaatgcgtctcccattgacgcggtgtctatttccactttgtata

SEQ ID NO: 794 - RNA, 46 nt, synthetic construct
  source 1..46: mol_type = other RNA
gtaatgcgtctcccattgacgcctgaacacatgcacggccacattg

SEQ ID NO: 795 - RNA, 45 nt, synthetic construct
  source 1..45: mol_type = other RNA
gtaatgcgtctcccattgacgccccagaggcaaatggctcccagg

SEQ ID NO: 796 - RNA, 45 nt, synthetic construct
  source 1..45: mol_type = other RNA
gtaatgcgtctcccattgacgcctcctcagttgtgagcccatgca

SEQ ID NO: 797 - RNA, 45 nt, synthetic construct
  source 1..45: mol_type = other RNA
gtaatgcgtctcccattgacgcgccagccttgccttgaccaatag

SEQ ID NO: 798 - RNA, 45 nt, synthetic construct
  source 1..45: mol_type = other RNA
gtaatgcgtctcccattgacgcaccaatagtcttagagtatccag

SEQ ID NO: 799 - RNA, 45 nt, synthetic construct
  source 1..45: mol_type = other RNA
gtaatgcgtctcccattgacgcccttgtcaaggctattggtcaag

SEQ ID NO: 800 - RNA, 45 nt, synthetic construct
  source 1..45: mol_type = other RNA
gtaatgcgtctcccattgacgcccttgaccaatagccttgacaag

SEQ ID NO: 801 - RNA, 45 nt, synthetic construct
  source 1..45: mol_type = other RNA
gtaatgcgtctcccattgacgcaccaatagccttgacaaggcaaa

SEQ ID NO: 802 - RNA, 45 nt, synthetic construct
  source 1..45: mol_type = other RNA
gtaatgcgtctcccattgacgcgtcaaggcaaggctggccaaccc

SEQ ID NO: 803 - RNA, 44 nt, synthetic construct
  source 1..44: mol_type = other RNA
gtaatgcgtctcccattgacgcgtcaagtttgccttgtcaaggc

SEQ ID NO: 804 - RNA, 45 nt, synthetic construct
  source 1..45: mol_type = other RNA
gtaatgcgtctcccattgacgctcaaggctattggtcaaggcaag

SEQ ID NO: 805 - RNA, 46 nt, synthetic construct
  source 1..46: mol_type = other RNA
gtaatgcgtctcccattgacgcacaaggcaaacttgaccaatagtc

SEQ ID NO: 806 - DNA, 25 bp, synthetic construct
  source 1..25: mol_type = other DNA
gaacgaatgttccgatgctctaatc

SEQ ID NO: 807 - DNA, 28 bp, synthetic construct
  source 1..28: mol_type = other DNA
aatgaggtagaatgctcagagttcaagt

SEQ ID NO: 808 - DNA, 26 bp, synthetic construct
  source 1..26: mol_type = other DNA
caactgttctcaggggacctattttc

SEQ ID NO: 809 - DNA, 26 bp, synthetic construct
  source 1..26: mol_type = other DNA
ctctactgtctgcccctaaatgatgc

SEQ ID NO: 810 - DNA, 28 bp, synthetic construct
  source 1..28: mol_type = other DNA
tgccatttaatttcagttaggagttttc

SEQ ID NO: 811 - DNA, 23 bp, synthetic construct
  source 1..23: mol_type = other DNA
cagacctccgagaagccagatgt

SEQ ID NO: 812 - DNA, 27 bp, synthetic construct
  source 1..27: mol_type = other DNA
caagacctgactctgtactcctgctct

SEQ ID NO: 813 - DNA, 25 bp, synthetic construct
  source 1..25: mol_type = other DNA
atttggatctctcctagcgttctgc

SEQ ID NO: 814 - DNA, 23 bp, synthetic construct
  source 1..23: mol_type = other DNA
acggctgacaaaagaagtcctgg

SEQ ID NO: 815 - DNA, 22 bp, synthetic construct
  source 1..22: mol_type = other DNA
catgggtagacaaccaggagcc

SEQ ID NO: 816 - protein, 1139 aa, synthetic construct
RFVVLSVNQFRCNPNAFRQASMKTDDEKKDTAIKSYTSWLLPNDRKEKDMVRSFLALDQGSRFFFDLMQAWYGGLTPDIIKSVTKERDLVDLWCAIYWFRPMKKELASHPIDRVDLAKTFQRYYGGPASDVVQEYLSASIGEDCCWNDCRQKYQEFCKNLGVDFTPDLKTLVREKLIAVALNDGSRAAISNLFGNGGKEDRNVKVNICKKILIALEKAGKSVKYVRDVQSIILKCANAKDREDFNRIYADKNGRPGTLLLLLDRKGENTYDAEVLKRYLRKTIKSKNAPLVWNYNQKLFEYIERGIRNLLRRTINYDAWAWGEMWRVGHTPLQTKATRNYHFAQEQLKRHSRIAAASQSSHAIAVALNEFFESPFFDGENPFTICPHHLGKNLEKLFKAWADEKNQTDEEDAGETAITDYCAEFKRGFDREPIRNLLRYVYASLRSKYSAKELIQAAKYNQQFERYQRQKAHPVTPDNQGYTWPEAVIKPSKAERKNRENSLDTRIWVSVKLLQKDGTWEKHHIPFYNLRFFEEVYAAASIAEDMPSPTFRNSRFGFKLPKLLNTHAKINKKKKDGKKDKKAVNAAKREARIYLAAKEGRLPAPSVPLDKLSAVIARVNGKFQVTVPVKFKVNKQPKYPLPQLGQIILGYAQNLTANHAWALFEVVTEDTPGAHFCRNQYLRVLKTGQVRSITKTKDGQEIDQLSYDGLEYKEYGEWRKQAKKFASQWYITTKVRKGKETTLLSQPALERFESIEKYKPALYRFNKEYARLLLAVIRRKTLNELEEIRPEIFRLVEQGFGICRFGSLSLSSFEAIRAAKAVIYGYFSTALKHDHNTPITDDERQARDPELFGLLNKLRTRRQNKGEEKINRAANALIRIALENKADFIRGAGDLPTTNKSVKKGQNSRSMDWLARGLFNKICQLAVMHNIIPTAANPHSTSHQDPFEHNKDFDDPQPAMKCRYAEFKIEDIGDSVLERLGTSLRNAKAGQTGAYYYKGAQDFLAHYGLQDIEEELKKCRRGRKANMPCWELQKRLIQKLGSKDAVVYIPMRGGRIYLATHSVTTGAKPIIFNGEEVWVSNAAEIAAVNIGLTIIPTSKKDEEHPDRENDDKAIRKSVRRPRKSGERRSGDKMPATARKT

SEQ ID NO: 817 - RNA, 70 nt, synthetic construct
  source 1..70: mol_type = other RNA
atgtgtttttgtcaaaagaccttttgtaatgcgtctcccattgacgccatgagcatgcagaggtgagtat

SEQ ID NO: 818 - RNA, 45 nt, synthetic construct; identical to SEQ ID NO: 783
  source 1..45: mol_type = other RNA

SEQ ID NO: 819 - RNA, 59 nt, synthetic construct
  source 1..59: mol_type = other RNA
gttgcaatcccaaggtaatgcgtctcccattgacgccatgagcatgcagaggtgagtat

SEQ ID NO: 820 - RNA, 45 nt, synthetic construct; identical to SEQ ID NO: 783
  source 1..45: mol_type = other RNA

SEQ ID NO: 821 - RNA, 25 nt, synthetic construct
  source 1..25: mol_type = other RNA
atgtgtttttgtcaaaagacctttt

SEQ ID NO: 822 - RNA, 14 nt, synthetic construct
  source 1..14: mol_type = other RNA
gttgcaatcccaag

SEQ ID NO: 823 - DNA, 29 bp, synthetic construct
  source 1..29: mol_type = other DNA
gcgtaacttaatccagactttcacacctt

SEQ ID NO: 824 - DNA, 26 bp, synthetic construct
  source 1..26: mol_type = other DNA
ggtcattcatcaccttccttaggaca

SEQ ID NO: 825 - RNA, 23 nt, synthetic construct
  source 1..23: mol_type = other RNA
ctggagcctgtgataaaagcaac

SEQ ID NO: 826 - RNA, 23 nt, synthetic construct
  source 1..23: mol_type = other RNA
aagctaacagttgcttttatcac

SEQ ID NO: 827 - RNA, 23 nt, synthetic construct
  source 1..23: mol_type = other RNA
cattgagatagtgtggggaaggg

SEQ ID NO: 828 - RNA, 23 nt, synthetic construct
  source 1..23: mol_type = other RNA
gcattgagatagtgtggggaagg

SEQ ID NO: 829 - RNA, 23 nt, synthetic construct
  source 1..23: mol_type = other RNA
agatagtgtggggaaggggcccc

SEQ ID NO: 830 - RNA, 23 nt, synthetic construct
  source 1..23: mol_type = other RNA
ggggccccttccccacactatct

SEQ ID NO: 831 - RNA, 23 nt, synthetic construct
  source 1..23: mol_type = other RNA
cctcttgggggccccttccccac

SEQ ID NO: 832 - RNA, 23 nt, synthetic construct
  source 1..23: mol_type = other RNA
tcctcttgggggccccttcccca

SEQ ID NO: 833 - RNA, 23 nt, synthetic construct
  source 1..23: mol_type = other RNA
gcagtatcctcttgggggcccct

SEQ ID NO: 834 - RNA, 23 nt, synthetic construct
  source 1..23: mol_type = other RNA
cccacactatctcaatgcaaata

SEQ ID NO: 835 - RNA, 23 nt, synthetic construct
  source 1..23: mol_type = other RNA
cagacagatatttgcattgagat

SEQ ID NO: 836 - RNA, 23 nt, synthetic construct
  source 1..23: mol_type = other RNA
agccagggaccgtttcagacaga

SEQ ID NO: 837 - RNA, 23 nt, synthetic construct
  source 1..23: mol_type = other RNA
acaaggcaaacttgaccaatagt

SEQ ID NO: 838 - RNA, 23 nt, synthetic construct
  source 1..23: mol_type = other RNA
atccctagccagccgccggcccc

SEQ ID NO: 839 - RNA, 23 nt, synthetic construct
  source 1..23: mol_type = other RNA
tagggtaccctcgtgcacccggg

SEQ ID NO: 840 - RNA, 23 nt, synthetic construct
  source 1..23: mol_type = other RNA
cgcggcagccactcgctggtatc

SEQ ID NO: 841 - RNA, 23 nt, synthetic construct
  source 1..23: mol_type = other RNA
gtccttgaggacagacaccactg

SEQ ID NO: 842 - RNA, 23 nt, synthetic construct
  source 1..23: mol_type = other RNA
atccttgttttccagtggtgtct

SEQ ID NO: 843 - RNA, 23 nt, synthetic construct
  source 1..23: mol_type = other RNA
gaagagttgaccatcagttatgg

SEQ ID NO: 844 - RNA, 23 nt, synthetic construct
  source 1..23: mol_type = other RNA
ctctactcctcaagaaaacacca

SEQ ID NO: 845 - RNA, 23 nt, synthetic construct
  source 1..23: mol_type = other RNA
acacagaaccctgccattctaaa

SEQ ID NO: 846 - RNA, 23 nt, synthetic construct
  source 1..23: mol_type = other RNA
agtctgcacaacaaaaacaagca

SEQ ID NO: 847 - RNA, 23 nt, synthetic construct
  source 1..23: mol_type = other RNA
gggtgataggtgcagatggtccg

SEQ ID NO: 848 - RNA, 23 nt, synthetic construct
  source 1..23: mol_type = other RNA
cccaacagttggtataaattgtg

SEQ ID NO: 849 - RNA, 23 nt, synthetic construct
  source 1..23: mol_type = other RNA
cagtctgcacaacaaaaacaagc

SEQ ID NO: 850 - RNA, 23 nt, synthetic construct
  source 1..23: mol_type = other RNA
aggtggtcccttagtttgcaaac

SEQ ID NO: 851 - RNA, 23 nt, synthetic construct
  source 1..23: mol_type = other RNA
ggggtgtacatggaagctacatc

SEQ ID NO: 852 - RNA, 23 nt, synthetic construct
  source 1..23: mol_type = other RNA
cctattaaagtacagtcccggag

SEQ ID NO: 853 - RNA, 23 nt, synthetic construct
  source 1..23: mol_type = other RNA
tagcggtaggtgttcctccggga

SEQ ID NO: 854 - RNA, 23 nt, synthetic construct
  source 1..23: mol_type = other RNA
cgtgctcgggtgcttcggccagg

SEQ ID NO: 855 - RNA, 23 nt, synthetic construct
  source 1..23: mol_type = other RNA
cccctccacggtaccgggcggat

SEQ ID NO: 856 - RNA, 23 nt, synthetic construct
  source 1..23: mol_type = other RNA
atccgcccggtaccgtggagggg

SEQ ID NO: 857 - RNA, 23 nt, synthetic construct
  source 1..23: mol_type = other RNA
gcagttgagcacgcgcaggctgc

SEQ ID NO: 858 - RNA, 23 nt, synthetic construct
  source 1..23: mol_type = other RNA
gccacgccggcatcccggccgct

SEQ ID NO: 859 - RNA, 23 nt, synthetic construct
  source 1..23: mol_type = other RNA
aggacgcggctgtacccacccgc

SEQ ID NO: 860 - RNA, 23 nt, synthetic construct
  source 1..23: mol_type = other RNA
ggccgctgtgtggacctctttgc

SEQ ID NO: 861 - RNA, 23 nt, synthetic construct
  source 1..23: mol_type = other RNA
tctacggcgtaggcccccaggac

SEQ ID NO: 862 - RNA, 23 nt, synthetic construct
  source 1..23: mol_type = other RNA
cccaagtctccaacatgcctaat

SEQ ID NO: 863 - RNA, 23 nt, synthetic construct
  source 1..23: mol_type = other RNA
cccacaccttcactggtccatta

SEQ ID NO: 864 - RNA, 23 nt, synthetic construct
  source 1..23: mol_type = other RNA
atgcttccccacaccttcactgg

SEQ ID NO: 865 - RNA, 23 nt, synthetic construct
  source 1..23: mol_type = other RNA
agcatcttgttacctctcttcat

SEQ ID NO: 866 - RNA, 23 nt, synthetic construct
  source 1..23: mol_type = other RNA
ggcaatgtgactgctgacaaaga

SEQ ID NO: 867 - RNA, 23 nt, synthetic construct
  source 1..23: mol_type = other RNA
agcacgcacacggccttcgtcgc

SEQ ID NO: 868 - RNA, 23 nt, synthetic construct
  source 1..23: mol_type = other RNA
aaggactgactgaaggcctgcat

SEQ ID NO: 869 - RNA, 23 nt, synthetic construct
  source 1..23: mol_type = other RNA
agggggaagccagcaccacaaag

SEQ ID NO: 870 - RNA, 23 nt, synthetic construct
  source 1..23: mol_type = other RNA
cctcttagtacaactatgacaag

SEQ ID NO: 871 - RNA, 23 nt, synthetic construct
  source 1..23: mol_type = other RNA
ccagtcatctcactctacctaat

SEQ ID NO: 872 - RNA, 23 nt, synthetic construct
  source 1..23: mol_type = other RNA
ccctctccccagtgcacgatgag

SEQ ID NO: 873 - RNA, 23 nt, synthetic construct
  source 1..23: mol_type = other RNA
agcagggagatgtcatcagcaaa

SEQ ID NO: 874 - RNA, 23 nt, synthetic construct
  source 1..23: mol_type = other RNA
ggtagagtgagatgactgggaaa

SEQ ID NO: 875 - RNA, 23 nt, synthetic construct
  source 1..23: mol_type = other RNA
cccctcaaccagtctgaagtgct

SEQ ID NO: 876 - RNA, 23 nt, synthetic construct
  source 1..23: mol_type = other RNA
agatcctgtccattgatctccac

SEQ ID NO: 877 - RNA, 23 nt, synthetic construct
  source 1..23: mol_type = other RNA
gcacgcacagtgacgttggggcc

SEQ ID NO: 878 - DNA, 65 bp, synthetic construct
  source 1..65: mol_type = other DNA
ccatggaacggctcggagatcatcattgcgnnnnnnngtgagcaagggcgaggagctgttcaccg

SEQ ID NO: 879 - DNA, 67 bp, synthetic construct
  source 1..67: mol_type = other DNA
ggtaccttgccgagcctctctagtagtaacgcnnnnnnncactcgttcccgctcctcgacaagtggc

SEQ ID NO: 880 - DNA, 24 bp, synthetic construct
  source 1..24: mol_type = other DNA
gtgagcaagggcgaggagctgttc

SEQ ID NO: 881 - DNA, 26 bp, synthetic construct
  source 1..26: mol_type = other DNA
ccttgccgagcctctagtagtaacgc

SEQ ID NO: 882 - DNA, 272 bp, synthetic construct
  source 1..272: mol_type = other DNA
ttattattttaaaaaacagcacaaaaggaaactcaccctaactgtaaagtaattgtgtgttttgagactataaatatgcatgcgagaaaagccttgtttgccaccatggaacggctcggagatcatcattgcgnnnnnnnaccgagtacaagcccacggtgcgcctcgccacccgcgcgacgacgtccccagggccgtacgcaccctcgccgccgcgttcgccgactaccccgccacgcgccacaccgtcgatccggaccgccacatcgagc

SEQ ID NO: 883 - DNA, 269 bp, synthetic construct
  source 1..269: mol_type = other DNA
aataataaaattttttgtcgtgttttcctttgagtgggattgacatttcattaacacacaaaactctgatatttatacgtacgctcttttcggaacaaacggtggtaccttgccgagcctctagtagtaacgcnnnnnntggctcatgttcgggtgccacgcggagcggtgggcgctgctgcaggggtcccggcatgcgtgggagcggcggcgcaagcggctgatggggcggtgcgcggtgtggcagctaggcctggcggtgtagctcg

SEQ ID NO: 884 - protein, 43 aa, synthetic construct
TEYKPTVRLATRDDVPRAVRTLAAAFADYPATRHTVDPDRHIE

SEQ ID NO: 885 - protein, 55 aa, synthetic construct
MERLGDHHCXXXPSTSPRCASPPATTSPGPYAPSPPRSPTTPPRATPSIRTATSS

SEQ ID NO: 886 - DNA, 20 bp, synthetic construct
  source 1..20: mol_type = other DNA
gatggggcggtgcgcggtgt

SEQ ID NO: 887 - DNA, 60 bp, synthetic construct
  source 1..60: mol_type = other DNA
ctccccattccatgagcatgcagaggtgagtatacagaccttcgagggttgttttggttt

SEQ ID NO: 888 - DNA, 60 bp, synthetic construct
  source 1..60: mol_type = other DNA
ctccccagtccatgagcatgcagaggtgagtatacagaccttcgagggttgttttggttt

SEQ ID NO: 889 - DNA, 46 bp, synthetic construct
  source 1..46: mol_type = other DNA
ctccccattccatgagcatgcagaccttcgagggttgttttggttt

SEQ ID NO: 890 - DNA, 55 bp, synthetic construct
  source 1..55: mol_type = other DNA
ctccccattccatgagcatgcagaggtgacagaccttcgagggttgttttggttt

SEQ ID NO: 891 - DNA, 58 bp, synthetic construct
  source 1..58: mol_type = other DNA
ctccccattccatgagcatgcagaggtgagtacagaccttcgagggttgttttggttt

SEQ ID NO: 892 - DNA, 50 bp, synthetic construct
  source 1..50: mol_type = other DNA
ctccccattccatgagcatgcagacagaccttcgagggttgttttggttt

SEQ ID NO: 893 - DNA, 51 bp, synthetic construct
  source 1..51: mol_type = other DNA
ctccccattccatgagcatgcagaggtgaccttcgagggttgttttggttt

SEQ ID NO: 894 - DNA, 54 bp, synthetic construct
  source 1..54: mol_type = other DNA
ctccccattccatgagcatgcagaggtgcagaccttcgagggttgttttggttt

SEQ ID NO: 895 - DNA, 58 bp, synthetic construct
  source 1..58: mol_type = other DNA
ctccccattccatgagcatgcagaggtgagtatagaccttcgagggttgttttggttt

SEQ ID NO: 896 - DNA, 56 bp, synthetic construct
  source 1..56: mol_type = other DNA
ctccccattccatgagcatgcagaggtgagtagaccttcgagggttgttttggttt

SEQ ID NO: 897 - DNA, 53 bp, synthetic construct
  source 1..53: mol_type = other DNA
ctccccattccatgagcatgcagaggtcagaccttcgagggttgttttggttt

SEQ ID NO: 898 - DNA, 52 bp, synthetic construct
  source 1..52: mol_type = other DNA
ctccccattccatgagcatgcagaggcagaccttcgagggttgttttggttt

SEQ ID NO: 899 - DNA, 57 bp, synthetic construct
  source 1..57: mol_type = other DNA
ctccccattccatgagcatgcagaggtgagtcagaccttcgagggttgttttggttt

SEQ ID NO: 900 - DNA, 49 bp, synthetic construct
  source 1..49: mol_type = other DNA
ctccccattccatgagcatgcagaagaccttcgagggttgttttggttt

SEQ ID NO: 901 - DNA, 48 bp, synthetic construct
  source 1..48: mol_type = other DNA
ctccccattccatgagcatgcagagaccttcgagggttgttttggttt

SEQ ID NO: 902 - DNA, 49 bp, synthetic construct
  source 1..49: mol_type = other DNA
ctccccattccatgagcatgcagcagaccttcgagggttgttttggttt

SEQ ID NO: 903 - DNA, 51 bp, synthetic construct
  source 1..51: mol_type = other DNA
ctccccattccatgagcatgcagagcagaccttcgagggttgttttggttt

SEQ ID NO: 904 - DNA, 53 bp, synthetic construct
  source 1..53: mol_type = other DNA
ctccccattccatgagcatgcagaggtgagaccttcgagggttgttttggttt

SEQ ID NO: 905 - DNA, 59 bp, synthetic construct
  source 1..59: mol_type = other DNA
ctccccattccatgagcatgcagaggtgagtatcagaccttcgagggttgttttggttt

SEQ ID NO: 906 - DNA, 54 bp, synthetic construct
  source 1..54: mol_type = other DNA
ctccccattccatgagcatgcagaggtgaagaccttcgagggttgttttggttt

SEQ ID NO: 907 - DNA, 56 bp, synthetic construct
  source 1..56: mol_type = other DNA
ctccccattccatgagcatgcagaggtgagcagaccttcgagggttgttttggttt

SEQ ID NO: 908 - DNA, 52 bp, synthetic construct
  source 1..52: mol_type = other DNA
ctccccattccatgagcatgcagaggtagaccttcgagggttgttttggttt

SEQ ID NO: 909 - DNA, 55 bp, synthetic construct
  source 1..55: mol_type = other DNA
ctccccattccatgagcatgcagaggtatcagaccttcgagggttgttttggttt

SEQ ID NO: 910 - DNA, 47 bp, synthetic construct
  source 1..47: mol_type = other DNA
ctccccattccatgagcatgcagaaccttcgagggttgttttggttt

SEQ ID NO: 911 - DNA, 54 bp, synthetic construct
  source 1..54: mol_type = other DNA
ctccccattccatgagcatgcagaggtacagaccttcgagggttgttttggttt

SEQ ID NO: 912 - DNA, 55 bp, synthetic construct
  source 1..55: mol_type = other DNA
ctccccattccatgagcatgcagaggtgagtgaccttcgagggttgttttggttt

SEQ ID NO: 913 - DNA, 60 bp, synthetic construct
  source 1..60: mol_type = other DNA
ctccccattccatgagcatgcagatgtgagtatacagaccttcgagggttgttttggttt

SEQ ID NO: 914 - DNA, 60 bp, synthetic construct
  source 1..60: mol_type = other DNA
ctccccattccatgatcatgcagaggtgagtatacagaccttcgagggttgttttggttt

SEQ ID NO: 915 - DNA, 60 bp, synthetic construct
  source 1..60: mol_type = other DNA
ctccccattccatgagcatgcagaggtgagtatacagaccttcgagtgttgttttggttt

SEQ ID NO: 916 - DNA, 52 bp, synthetic construct
  source 1..52: mol_type = other DNA
ctccccattccatgagcatgcagatacagaccttcgagggttgttttggttt

SEQ ID NO: 917 - RNA, 23 nt, synthetic construct
  source 1..23: mol_type = other RNA
ccgcagttaccctctgcgtaatg

SEQ ID NO: 918 - RNA, 22 nt, synthetic construct
  source 1..22: mol_type = other RNA
cgcagttaccctctgcgtaatg

SEQ ID NO: 919 - DNA, 300 bp, synthetic construct
  source 1..300: mol_type = other DNA
tataagcagagctcgtttagtgaaccgtcagatcgcctggagacgccatccacgctgttttgacctccatagaagattctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagct

SEQ ID NO: 920 - DNA, 303 bp, synthetic construct
  source 1..303: mol_type = other DNA
atattcgtctcgagcaaatcacttggcagtctagcggacctctgcggtaggtgcgacaaaactggaggtatcttctaagatctcgctcttttcggaacaaacggtggtaccttgccgagcctctagtagtaacgcagcgctccactcgttcccgctcctcgacaagtggccccaccaccacgggtaggaccagctcgacctgccgctgcatttgccggtgttcaagtcgcacaggccgctcccgctcccgctacggtggatgccgttcgactgggacttcaagtagacgtggtggccgttcga

SEQ ID NO: 921 - protein, 53 aa, synthetic construct
VSKGEELFTGVVPILVELDGDVNGHKFSVSGEGEGDATYGKLTLKFICTTGKL

SEQ ID NO: 922 - DNA, 300 bp, synthetic construct
  source 1..300: mol_type = other DNA
tataagcagagctcgtttagtgaaccgtcagatcgcctggagacgccatccacgctgttttgacctccatagaagattctagagcgagaaaagccttgtttgccaccatggaacggctcggagatcatcattgcgnaangtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcc

SEQ ID NO: 923 - DNA, 300 bp, synthetic construct
  source 1..300: mol_type = other DNA
atattcgtctcgagcaaatcacttggcagtctagcggacctctgcggtaggtgcgacaaaactggaggtatcttctaagatctcgctcttttcggaacaaacggtggtaccttgccgagcctctagtagtaacgcnttncactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgg

SEQ ID NO: 924 - protein, 54 aa, synthetic construct
VSKGEELFTGVVPILVELDGDVNGHKFSVSGEGEGDATYGKLTLKFICTTGKLP

SEQ ID NO: 925 - DNA, 79 bp, synthetic construct
  source 1..79: mol_type = other DNA
atctccccattccatgagcatgcagaggtgagtatacagaccttcgagggttgttttggttttggtttttgcttttggc

SEQ ID NO: 926 - DNA, 65 bp, synthetic construct
  source 1..65: mol_type = other DNA
atctccccattccatgagcatgcagaccttcgagggttgttttggttttggtttttgcttttggc

SEQ ID NO: 927 - DNA, 58 bp, synthetic construct
  source 1..58: mol_type = other DNA
atctccccattccatgagcatgcagagggttgttttggttttggtttttgcttttggc

SEQ ID NO: 928 - DNA, 69 bp, synthetic construct
  source 1..69: mol_type = other DNA
atctccccattccatgagcatgcagacagaccttcgagggttgttttggttttggtttttgcttttggc

SEQ ID NO: 929 - DNA, 70 bp, synthetic construct
  source 1..70: mol_type = other DNA
atctccccattccatgagcatgcagaggtgaccttcgagggttgttttggttttggtttttgcttttggc

SEQ ID NO: 930 - DNA, 71 bp, synthetic construct
  source 1..71: mol_type = other DNA
atctccccattccatgagcatgcagatacagaccttcgagggttgttttggttttggtttttgcttttggc

SEQ ID NO: 931 - DNA, 73 bp, synthetic construct
  source 1..73: mol_type = other DNA
atctccccattccatgagcatgcagaggtacagaccttcgagggttgttttggttttggtttttgcttttggc

SEQ ID NO: 932 - DNA, 77 bp, synthetic construct
  source 1..77: mol_type = other DNA
atctccccattccatgagcatgcagaggtgagtacagaccttcgagggttgttttggttttggtttttgcttttggc

SEQ ID NO: 933 - DNA, 73 bp, synthetic construct
  source 1..73: mol_type = other DNA
atctccccattccatgagcatgcagaggtgcagaccttcgagggttgttttggttttggtttttgcttttggc

SEQ ID NO: 934 - DNA, 72 bp, synthetic construct
  source 1..72: mol_type = other DNA
atctccccattccatgagcatgcagaggtcagaccttcgagggttgttttggttttggtttttgcttttggc

SEQ ID NO: 935 - DNA, 74 bp, synthetic construct
  source 1..74: mol_type = other DNA
atctccccattccatgagcatgcagaggtgacagaccttcgagggttgttttggttttggtttttgcttttggc

SEQ ID NO: 936 - DNA, 67 bp, synthetic construct
  source 1..67: mol_type = other DNA
atctccccattccatgagcatgcagagaccttcgagggttgttttggttttggtttttgcttttggc

SEQ ID NO: 937 - DNA, 74 bp, synthetic construct
  source 1..74: mol_type = other DNA
atctccccattccatgagcatgcagaggtatcagaccttcgagggttgttttggttttggtttttgcttttggc

SEQ ID NO: 938 - DNA, 70 bp, synthetic construct
  source 1..70: mol_type = other DNA
atctccccattccatgagcatgcagagcagaccttcgagggttgttttggttttggtttttgcttttggc

SEQ ID NO: 939 - DNA, 68 bp, synthetic construct
  source 1..68: mol_type = other DNA
atctccccattccatgagcatgcagcagaccttcgagggttgttttggttttggtttttgcttttggc

SEQ ID NO: 940 - DNA, 70 bp, synthetic construct
  source 1..70: mol_type = other DNA
atctccccattccatgagcatgcagtacagaccttcgagggttgttttggttttggtttttgcttttggc

SEQ ID NO: 941 - DNA, 71 bp, synthetic construct
  source 1..71: mol_type = other DNA
atctccccattccatgagcatgcagaggcagaccttcgagggttgttttggttttggtttttgcttttggc

SEQ ID NO: 942 - DNA, 72 bp, synthetic construct
  source 1..72: mol_type = other DNA
atctccccattccatgagcatgcagagtacagaccttcgagggttgttttggttttggtttttgcttttggc

SEQ ID NO: 943 - DNA, 66 bp, synthetic construct
  source 1..66: mol_type = other DNA
atctccccattccatgagcatgcagaaccttcgagggttgttttggttttggtttttgcttttggc

SEQ ID NO: 944 - DNA, 56 bp, synthetic construct
  source 1..56: mol_type = other DNA
atctccccattccatgaccttcgagggttgttttggttttggtttttgcttttggc

SEQ ID NO: 945 - DNA, 56 bp, synthetic construct
  source 1..56: mol_type = other DNA
atctccccattccatgagcatgcagggttgttttggttttggtttttgcttttggc

SEQ ID NO: 946 - DNA, 74 bp, synthetic construct
  source 1..74: mol_type = other DNA
atctccccattccatgagcatgcagaggttacagaccttcgagggttgttttggttttggtttttgcttttggc

SEQ ID NO: 947 - DNA, 63 bp, synthetic construct
  source 1..63: mol_type = other DNA
atctccccattccatgagcatgcagattcgagggttgttttggttttggtttttgcttttggc

SEQ ID NO: 948 - DNA, 39 bp, synthetic construct
  source 1..39: mol_type = other DNA
ccttcgagggttgttttggttttggtttttgcttttggc

SEQ ID NO: 949 - DNA, 70 bp, synthetic construct
  source 1..70: mol_type = other DNA
atctccccattccatgagcatgcagaacagaccttcgagggttgttttggttttggtttttgcttttggc

SEQ ID NO: 950 - DNA, 38 bp, synthetic construct
  source 1..38: mol_type = other DNA
cttcgagggttgttttggttttggtttttgcttttggc

SEQ ID NO: 951 - DNA, 57 bp, synthetic construct
  source 1..57: mol_type = other DNA
atctccccattccatgagcatgcagaggttgttttggttttggtttttgcttttggc

SEQ ID NO: 952 - DNA, 64 bp, synthetic construct
  source 1..64: mol_type = other DNA
atctccccattccatgagcatgcagacttcgagggttgttttggttttggtttttgcttttggc

SEQ ID NO: 953 - DNA, 69 bp, synthetic construct
  source 1..69: mol_type = other DNA
atctccccattccatgagcatgcagaggtaccttcgagggttgttttggttttggtttttgcttttggc

SEQ ID NO: 954 - DNA, 30 bp, synthetic construct
  source 1..30: mol_type = other DNA
gttgttttggttttggtttttgcttttggc

SEQ ID NO: 955 - DNA, 75 bp, synthetic construct
  source 1..75: mol_type = other DNA
atctccccattccatgagcatgcagaggtgtacagaccttcgagggttgttttggttttggtttttgcttttggc

SEQ ID NO: 956 - DNA, 71 bp, synthetic construct
  source 1..71: mol_type = other DNA
atctccccattccatgagcatgcagaggtagaccttcgagggttgttttggttttggtttttgcttttggc

SEQ ID NO: 957 - DNA, 65 bp, synthetic construct
  source 1..65: mol_type = other DNA
atctccccattccatgagcatgcagaggttcgagggttgttttggttttggtttttgcttttggc

SEQ ID NO: 958 - DNA, 60 bp, synthetic construct
  source 1..60: mol_type = other DNA
atctccccattccatgagcatgcttcgagggttgttttggttttggtttttgcttttggc

SEQ ID NO: 959 - DNA, 49 bp, synthetic construct
  source 1..49: mol_type = other DNA
atctccccattccatgagggttgttttggttttggtttttgcttttggc

SEQ ID NO: 960 - DNA, 68 bp, synthetic construct
  source 1..68: mol_type = other DNA
atctccccattccatgagcatgcagaagaccttcgagggttgttttggttttggtttttgcttttggc

SEQ ID NO: 961 - DNA, 75 bp, synthetic construct
  source 1..75: mol_type = other DNA
atctccccattccatgagcatgcagaggtatacagaccttcgagggttgttttggttttggtttttgcttttggc

SEQ ID NO: 962 - DNA, 76 bp, synthetic construct
  source 1..76: mol_type = other DNA
atctccccattccatgagcatgcagaggtgatacagaccttcgagggttgttttggttttggtttttgcttttggc

SEQ ID NO: 963 - DNA, 68 bp, synthetic construct
  source 1..68: mol_type = other DNA
atctccccattccatgagcatgcagaggaccttcgagggttgttttggttttggtttttgcttttggc

SEQ ID NO: 964 - DNA, 78 bp, synthetic construct
  source 1..78: mol_type = other DNA
atctccccattccatgagcatgcagaggtgagtatcagaccttcgagggttgttttggttttggtttttgcttttggc

SEQ ID NO: 965 - DNA, 24 bp, synthetic construct
  source 1..24: mol_type = other DNA
atctccccattccatgagcatgca

SEQ ID NO: 966 - DNA, 69 bp, synthetic construct
  source 1..69: mol_type = other DNA
atctccccattccatgagcatgcagaggtgccttcgagggttgttttggttttggtttttgcttttggc

SEQ ID NO: 967 - DNA, 77 bp, synthetic construct
  source 1..77: mol_type = other DNA
atctccccattccatgagcatgcagaggtgagtatagaccttcgagggttgttttggttttggtttttgcttttggc

SEQ ID NO: 968 - DNA, 68 bp, synthetic construct
  source 1..68: mol_type = other DNA
atctccccattccatgagcatgcagaggtccttcgagggttgttttggttttggtttttgcttttggc

SEQ ID NO: 969 - DNA, 67 bp, synthetic construct
  source 1..67: mol_type = other DNA
atctccccattccatgagcatgcagaggtcttcgagggttgttttggttttggtttttgcttttggc

SEQ ID NO: 970 - DNA, 67 bp, synthetic construct
  source 1..67: mol_type = other DNA
atctccccattccatgagcatgcacagaccttcgagggttgttttggttttggtttttgcttttggc

SEQ ID NO: 971 - DNA, 62 bp, synthetic construct
  source 1..62: mol_type = other DNA
atctccccattccatgagcatgaccttcgagggttgttttggttttggtttttgcttttggc

SEQ ID NO: 972 - DNA, 68 bp, synthetic construct
  source 1..68: mol_type = other DNA
atctccccattccatgagcatgcagaggtacttcgagggttgttttggttttggtttttgcttttggc

SEQ ID NO: 973 - DNA, 72 bp, synthetic construct
  source 1..72: mol_type = other DNA
atctccccattccatgagcatgcagaggacagaccttcgagggttgttttggttttggtttttgcttttggc

SEQ ID NO: 974 - DNA, 61 bp, synthetic construct
  source 1..61: mol_type = other DNA
atctccccattccatgagcatgccttcgagggttgttttggttttggtttttgcttttggc

SEQ ID NO: 975 - protein, 1139 aa, synthetic construct
RFRVLSVNQFRCNPNAFRQASMKTDDEKKDTAIKSYTSWLLPNDRKEKDMVRSFLALDQGSRFFFDLMQAWYGGLTPDIIKSVTKERDLVDLWCAIYWFRPMKKELASHPIDRVDLAKTFQRYYGGPASDVVQEYLSASIGEDCCWNDCRQKYQEFCKNLGVDFTPDLKTLVREKLIAVALNDGSRAAISNLFGNGGKEDRNVKVNICKKILIALEKAGKSVKYVRDVQSIILKCANAKDREDFNRIYADKNGRPGTLLLLLDRKGENTYDAEVLKRYLRKTIKSKNAPLVWNYNQKLFEYIERGIRNLLRRTINYDAWAWGEMWRVGHTPLQTKATRNYHFAQEQLKRHSRIAAASQSSHAIAVALNEFFESPFFDGENPFTICPHHLGKNLEKLFKAWADEKNQTDEEDAGETAITDYCAEFKRGFDREPIRNLLRYVYASLRSKYSAKELIQAAKYNQQFERYQRQKAHPVTPDNQGYTWPEAVIKPSKAERKNRENSLDTRIWVSVKLLQKDGTWEKHHIPFYNLRFFEEVYAAASIAEDMPSPTFRNSRFGFKLPKLLNTHAKINKKKKDGKKDKKAVNAAKREARIYLAAKEGRLPAPSVPLDKLSAVIARVNGKFQVTVPVKFKVNKQPKYPLPQLGQIILGYAQNLTANHAWALFEVVTEDTPGAHFCRNQYLRVLKTGQVRSITKTKDGQEIDQLSYDGLEYKEYGEWRKQAKKFASQWYITTKVRKGKETTLLSQPALERFESIEKYKPALYRFNKEYARLLLAVIRRKTLNELEEIRPEIFRLVEQGFGICRFGSLSLSSFEAIRAAKAVIYGYFSTALKHDHNTPITDDERQARDPELFGLLNKLRTRRQNKGEEKINRAANALIRIALENKADFIRGAGDLPTTNKSVKKGQNSRSMDWLARGLFNKICQLAVMHNIIPTAANPHSTSHQDPFEHNKDFDDPQPAMKCRYAEFKIEDIGDSVLERLGTSLRNAKAGQTGAYYYKGAQDFLAHYGLQDIEEELKKCRRGRKANMPCWELQKRLIQKLGSKDAVVYIPMRGGRIYLATHSVTTGAKPIIFNGEEVWVSNAAEIAAVNIGLTIIPTSKKDEEHPDRENDDKAIRKSVRRPRKSGERRSGDKMPATARKT

SEQ ID NO: 976 - DNA, 10255 bp, synthetic construct
  source 1..10255: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccgggccaccatgggaaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactctggaggatctagcggaggatcctctggcagcgagacaccaggaacaagcgagtcagcaacaccagagagcagtggcggcagcagcggcggcagccccaagaaaaaacgcaaggtgggaagcaggttcagggtgctgagcgtgaatcagttcagatgcaaccccaacgccttcagacaagcctccatgaaaaccgacgacgagaagaaggacaccgccatcaagagctacacaagctggctgctgcccaacgacagaaaggagaaggacatggtgcggagcttcctggccctggaccaaggcagcagattctttttcgacctgatgcaagcctggtacggcggcctcacccccgacatcatcaagagcgtgaccaaggagcgggacctggtggacctgtggtgcgccatctactggttcagacccatgaagaaggagctggcctcccaccccatcgacagagtggacctggccaagacctttcagagatactacggcggccccgcctccgacgtggtgcaagagtacctgagcgcctccatcggcgaggactgctgctggaacgactgcagacagaagtaccaagagttctgcaagaacctcggggtggacttcacccccgacctcaagaccctggtgagagagaagctgatcgccgtggccctgaacgacggcagcagagccgccatcagcaacctgttcggcaacggcggcaaggaggacagaaacgtgaaggtgaacatctgcaagaagatcctgattgctctggagaaggccggcaaatccgtgaagtacgtgcgggacgtgcagagcatcatcctgaagtgcgccaacgccaaggacagagaggacttcaacagaatctacgccgacaagaacggcagacccggcaccctgctcctgctcctggacagaaagggcgagaacacctacgacgccgaggtgctgaagagatacctgagaaagaccatcaagagcaagaacgcccccctggtgtggaactacaatcagaagctgttcgagtacatcgagagaggcatccggaatctgctgagaagaaccatcaactacgacgcctgggcctggggcgagatgtggagagtgggccacacccccctgcagaccaaggccacaagaaactaccacttcgcccaagagcagctgaagagacacagcagaatcgccgccgcttcccaaagcagccacgccatcgccgtcgctctgaacgagttcttcgagagccccttcttcgacggcgagaaccccttcaccatctgcccccaccacctgggcaagaacctggaaaagctgttcaaggcctgggccgacgaaaaaaatcagaccgacgaggaggacgccggcgaaaccgccatcaccgactactgcgccgagttcaagagaggcttcgacagagagcccattagaaacctgctgagatacgtgtacgcttccctgagaagcaagtacagcgccaaggagctgatccaagccgccaagtacaatcagcagttcgagagatatcagagacagaaggcccaccccgtgacccccgacaaccaaggctacacctggcccgaggccgtgatcaagcctagcaaggccgagagaaagaacagagagaacagcctggacacaagaatctgggtgagcgtgaagctgctgcaaaaggacggcacctgggagaagcaccacatccccttctacaacctgagattcttcgaagaggtgtacgccgctgcctccatcgccgaggacatgcctagccccaccttcagaaacagcagattcggcttcaagctgcccaagctgctgaacacccacgccaagatcaacaagaaaaagaaggacggcaagaaggacaagaaggccgtgaacgccgccaagagagaggctagaatctacctcgctgccaaggagggcagactgcccgccccctccgtgcccctggacaagctgagcgccgtgatcgctagagtgaacggcaagttccaagtgaccgtgcccgtgaagttcaaggtgaacaagcagcccaagtaccccctgcctcagctggggcagatcatcctgggctacgcccagaacctgaccgccaaccacgcctgggccctgttcgaggtggtgaccgaggacacccccggcgcccacttctgcagaaatcagtacctgagagtgctcaagaccggccaagtgagaagcatcaccaagaccaaggacggccaagagatcgatcagctgagctacgacggcctggagtacaaggagtacggcgagtggagaaagcaagccaagaagttcgcctcccaatggtacatcaccaccaaggtgagaaagggcaaggaaaccaccctgctgagccaacccgccctggagagattcgagagcatcgagaagtacaagcccgccctgtacagattcaacaaggaatacgcccggctgctcctggccgtcatcagaagaaagaccctgaacgagctggaggagatcagacccgagatcttcagactggtggagcaaggcttcggcatctgcagattcggcagcctgagcctgagcagcttcgaggccatcagagccgccaaggccgtgatctacggctacttcagcaccgccctgaagcacgaccacaacacccccatcacagatgacgagagacaagccagagaccccgagctgttcggcctgctgaacaagctgagaaccaggagacagaacaagggcgaggagaagatcaacagagccgctaacgccctgatcagaatcgccctggagaacaaggccgacttcatcagaggcgccggcgacctgcccaccaccaacaagagcgtcaagaaggggcagaacagcagaagcatggactggctggctagaggcctgttcaacaagatctgtcagctggccgtgatgcacaacatcattcccaccgccgccaacccccacagcacaagccaccaagaccccttcgagcacaacaaggacttcgacgaccctcagcccgccatgaagtgcagatacgctgagttcaagatcgaggacatcggcgacagcgtgctggagagactgggcacctccctgagaaatgccaaggccgggcagaccggcgcctactattacaagggcgcccaagacttcctggcccactacggcctgcaagacatcgaggaagagctgaagaagtgcagaagaggcagaaaggccaacatgccctgctgggagctgcagaagagactgattcagaagctgggcagcaaggacgccgtggtgtacatccccatgagaggcggccggatctacctggctacacacagcgtgaccaccggcgccaagcccatcatcttcaacggcgaagaggtgtgggtgagcaacgccgccgagatcgccgccgtgaacatcggcctgaccatcatccccacaagcaagaaggacgaggagcaccccgacagagagaacgacgacaaggccatcagaaagagcgtcagacggcctagaaagagcggcgagagaagaagcggcgacaagatgcccgccaccgctagaaagaccacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagcctgctgccaaacgcgttaaactagacgactataaggaccacgacggagactacaaggatcatgatattgattacaaagacgatgacgataagtgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 977 - RNA, 22 nt, synthetic construct
  source 1..22: mol_type = other RNA
gtaatgcggctcccattgccgc

SEQ ID NO: 978 - RNA, 22 nt, synthetic construct
  source 1..22: mol_type = other RNA
gtaatgcgcctcccattggcgc

SEQ ID NO: 979 - RNA, 22 nt, synthetic construct
  source 1..22: mol_type = other RNA
gtaatgcgtctcccagtgacgc

SEQ ID NO: 980 - RNA, 22 nt, synthetic construct
  source 1..22: mol_type = other RNA
gtaatgcgtctcccatggacgc

SEQ ID NO: 981 - protein, 35 aa, synthetic construct
GVEDDSRLLLGIYEHGYGNWELIKTDPELKLTDKL

SEQ ID NO: 982 - protein, 225 aa, synthetic construct
MSCESSMVLGYWDIRGLAHAIRLLLEFTDTSYEEKRYTCGEAPDYDRSQWLDVKFKLDLDFPNLPYLLDGKNKITQSNAILRYIARKHNMCGETEEEKIRVDIIENQVMDFRTQLIRLCYSSDHEKLKPQYLEELPGQLKQFSMFLGKFSWFAGEKLTFVDFLTYDILDQNRIFDPKCLDEFPNLKAFMCRFEALEKIAAYLQSDQFCKMPINNKMAQWGNKPVC

SEQ ID NO: 983 - protein, 272 aa, synthetic construct
MAAEEPQQQKQEPLGSDSEGVNCLAYDEAIMAQQDRIQQEIAVQNPLVSERLELSVLYKEYAEDDNIYQQKIKDLHKKYSYIRKTRPDGNCFYRAFGFSHLEALLDDSKELQRFKAVSAKSKEDLVSQGFTEFTIEDFHNTFMDLIEQVEKQTSVADLLASFNDQSTSDYLVVYLRLLTSGYLQRESKFFEHFIEGGRTVKEFCQQEVEPMCKESDHIHIIALAQALSVSIQVEYMDRGEGGTTNPHIFPEGSEPKVYLLYRPGHYDILYKE

SEQ ID NO: 984 - protein, 291 aa, synthetic construct
MACLSPSQLQKFQQDGFLVLEGFLSAEECVAMQQRIGEIVAEMDVPLHCRTEFSTQEEEQLRAQGSTDYFLSSGDKIRFFFEKGVFDEKGNFLVPPEKSINKIGHALHAHDPVFKSITHSFKVQTLARSLGLQMPVVVQSMYIFKQPHFGGEVSPHQDASFLYTEPLGRVLGVWIAVEDATLENGCLWFIPGSHTSGVSRRMVRAPVGSAPGTSFLGSEPARDNSLFVPTPVQRGALVLIHGEVVHKSKQNLSDRSRQAYTFHLMEASGTTWSPENWLQPTAELPFPQLYT

SEQ ID NO: 985 - protein, 276 aa, synthetic construct
MDSSREPTLGRLDAAGFWQVWQRFDADEKGYIEEKELDAFFLHMLMKLGTDDTVMKANLHKVKQQFMTTQDASKDGRIRMKELAGMFLSEDENFLLLFRRENPLDSSVEFMQIWRKYDADSSGFISAAELRNFLRDLFLHHKKAISEAKLEEYTGTMMKIFDRNKDGRLDLNDLARILALQENFLLQFKMDACSTEERKRDFEKIFAYYDVSKTGALEGPEVDGFVKDMMELVQPSISGVDLDKFREILLRHCDVNKDGKIQKSELALCLGLKINP

SEQ ID NO: 986 - protein, 134 aa, synthetic construct
MDVFMKGLSMAKEGVVAAAEKTKQGVTEAAEKTKEGVLYVGSKTREGVVQGVASVAEKTKEQASHLGGAVFSGAGNIAAATGLVKREEFPTDLKPEEVAQEAAEEPLIEPLMEPEGESYEDPPQEEYQEYEPEA

SEQ ID NO: 987 - protein, 28 aa, synthetic construct
MFRRPVLQVLRQFVRHESETTTSLVLER

SEQ ID NO: 988 - protein, 63 aa, synthetic construct
ATVKFKYKGEEKEVDISKIKKVWRVGKMISFTYDEGGGKTGRGAVSEKDAPKELLQMLEKQKK

SEQ ID NO: 989 - protein, 244 aa, synthetic construct
MEVTGVSAPTVTVFISSSLNTFRSEKRYSRSLTIAEFKCKLELLVGSPASCMELELYGVDDKFYSKLDQEDALLGSYPVDDGCRIHVIDHSGARLGEYEDVSRVEKYTISQEAYDQRQDTVRSFLKRSKLGRYNEEERAQQEAEAAQRLAEEKAQASSIPVGSRCEVRAAGQSPRRGTVMYVGLTDFKPGYWIGVRYDEPLGKNDGSVNGKRYFECQAKYGAFVKPAVVTVGDFPEEDYGLDEI

SEQ ID NO: 990 - protein, 270 aa, synthetic construct
MARFALTVVRHGETRFNKEKIIQGQGVDEPLSETGFKQAAAAGIFLNNVKFTHAFSSDLMRTKQTMHGILERSKFCKDMTVKYDSRLRERKYGVVEGKALSELRAMAKAAREECPVFTPPGGETLDQVKMRGIDFFEFLCQLILKEADQKEQFSQGSPSNCLETSLAEIFPLGKNHSSKVNSDSGIPGLAASVLVVSHGAYMRSLFDYFLTDLKCSLPATLSRSELMSVTPNTGMSLFIINFEEGREVKPTVQCICMNLQDHLNGLTETR

SEQ ID NO: 991 - protein, 16 aa, synthetic construct
ATGWSAFFIDGKWTEA

SEQ ID NO: 992 - protein, 223 aa, synthetic construct [sequence omitted]

SEQ ID NO: 993 - protein, 245 aa, synthetic construct
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 994 - protein, 119 aa, synthetic construct
NTYSITLPWPPSNNRYYRHNRGRTHVSAEGQAYRDNVARIIKNAMLDIGLAMPVKIRIECHMPDRRRRNLDNLQKAAFDALTKAGFWLDDAQVVDYRVVKMPVTKGGRLELTITEMGNE

SEQ ID NO: 995 - protein, 87 aa, synthetic construct
MDNVVLIGKKPVMNYVVAVLTQLTSNDEVIIKARGKAINKAVDVAEMIRNRFIKDIKIKKIEIGTDKVKNPDGREVNVSTIEIVLAK

SEQ ID NO: 996 - protein, 85 aa, synthetic construct
MYIVVVYDVGVERVNKVKKFLRMHLNWVQNSVFEGEVTLAEFERIKEGLKKIIDENSDSVIIYKLRSMPPRETLGIEKNPIEEII

SEQ ID NO: 997 - protein, 25 aa, synthetic construct
MKKSIRRSGGKSKGARLWYVGGTQY

SEQ ID NO: 998 - protein, 78 aa, synthetic construct
MSYNYVVTAQKPTAVNGCVTGHFTSAEDLNLLIAKNTRLEIYVVTAEGLRPVKEVGMYGKIAVMELFRPKGEKNLENE

SEQ ID NO: 999 - protein, 96 aa, synthetic construct
MSELGNLETTVTGKIKRFNNGGGYYYTTVVSPAADAYSFPPVIRIKSKKSLGRVGDEIADIHCRITGYERSFPYTDKQTGEQSRGFNVDMLLELLE

SEQ ID NO: 1000 - protein, 32 aa, synthetic construct
MNKGELVDAVMAKATVTKKQADAFILALDIVA

SEQ ID NO: 1001 - protein, 96 aa, synthetic construct
MNKKSKQQEKLYNFIIAKSFQQPVGSTFTYGELRKKYNVVCSTNDQREVGRRFAYWIKYTPGLPFKIVGTKNGSLLYQKIGINPCNNSTPSKGGDC

SEQ ID NO: 1002 - protein, 275 aa, synthetic construct
ASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 1003 - DNA, 123 bp, synthetic construct
  source 1..123: mol_type = other DNA
ggtggaggaggtgggtctggcgtggaggacgacagccgcctgctgctgggcatctacgagcacggctacggcaactgggagctgatcaagaccgaccccgagctgaagctgaccgacaagctg

SEQ ID NO: 1004 - DNA, 39 bp, synthetic construct
  source 1..39: mol_type = other DNA
gcaaaaaagaaaaagctcgagggtggaggaggtgggtct

SEQ ID NO: 1005 - DNA, 63 bp, synthetic construct
  source 1..63: mol_type = other DNA
atctggaacatcgtatgggtactccagcttgtcggtcagcttcagctcggggtcggtcttgat

SEQ ID NO: 1006 - DNA, 9071 bp, synthetic construct
  source 1..9071: mol_type = other DNA
cggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacagattcgtggtgctgagcgtgaatcagttcagatgcaaccccaacgccttcagacaagcctccatgaaaaccgacgacgagaagaaggacaccgccatcaagagctacacaagctggctgctgcccaacgacagaaaggagaaggacatggtgcggagcttcctggccctggaccaaggcagcagattctttttcgacctgatgcaagcctggtacggcggcctcacccccgacatcatcaagagcgtgaccaaggagcgggacctggtggacctgtggtgcgccatctactggttcagacccatgaagaaggagctggcctcccaccccatcgacagagtggacctggccaagacctttcagagatactacggcggccccgcctccgacgtggtgcaagagtacctgagcgcctccatcggcgaggactgctgctggaacgactgcagacagaagtaccaagagttctgcaagaacctcggggtggacttcacccccgacctcaagaccctggtgagagagaagctgatcgccgtggccctgaacgacggcagcagagccgccatcagcaacctgttcggcaacggcggcaaggaggacagaaacgtgaaggtgaacatctgcaagaagatcctgattgctctggagaaggccggcaaatccgtgaagtacgtgcgggacgtgcagagcatcatcctgaagtgcgccaacgccaaggacagagaggacttcaacagaatctacgccgacaagaacggcagacccggcaccctgctcctgctcctggacagaaagggcgagaacacctacgacgccgaggtgctgaagagatacctgagaaagaccatcaagagcaagaacgcccccctggtgtggaactacaatcagaagctgttcgagtacatcgagagaggcatccggaatctgctgagaagaaccatcaactacgacgcctgggcctggggcgagatgtggagagtgggccacacccccctgcagaccaaggccacaagaaactaccacttcgcccaagagcagctgaagagacacagcagaatcgccgccgcttcccaaagcagccacgccatcgccgtcgctctgaacgagttcttcgagagccccttcttcgacggcgagaaccccttcaccatctgcccccaccacctgggcaagaacctggaaaagctgttcaaggcctgggccgacgaaaaaaatcagaccgacgaggaggacgccggcgaaaccgccatcaccgactactgcgccgagttcaagagaggcttcgacagagagcccattagaaacctgctgagatacgtgtacgcttccctgagaagcaagtacagcgccaaggagctgatccaagccgccaagtacaatcagcagttcgagagatatcagagacagaaggcccaccccgtgacccccgacaaccaaggctacacctggcccgaggccgtgatcaagcctagcaaggccgagagaaagaacagagagaacagcctggacacaagaatctgggtgagcgtgaagctgctgcaaaaggacggcacctgggagaagcaccacatccccttctacaacctgagattcttcgaagaggtgtacgccgctgcctccatcgccgaggacatgcctagccccaccttcagaaacagcagattcggcttcaagctgcccaagctgctgaacacccacgccaagatcaacaagaaaaagaaggacggcaagaaggacaagaaggccgtgaacgccgccaagagagaggctagaatctacctcgctgccaaggagggcagactgcccgccccctccgtgcccctggacaagctgagcgccgtgatcgctagagtgaacggcaagttccaagtgaccgtgcccgtgaagttcaaggtgaacaagcagcccaagtaccccctgcctcagctggggcagatcatcctgggctacgatcagaacctgaccgccaaccacgcctgggccctgttcgaggtggtgaccgaggacacccccggcgcccacttctgcagaaatcagtacctgagagtgctcaagaccggccaagtgagaagcatcaccaagaccaaggacggccaagagatcgatcagctgagctacgacggcctggagtacaaggagtacggcgagtggagaaagcaagccaagaagttcgcctcccaatggtacatcaccaccaaggtgagaaagggcaaggaaaccaccctgctgagccaacccgccctggagagattcgagagcatcgagaagtacaagcccgccctgtacagattcaacaaggaatacgcccggctgctcctggccgtcatcagaagaaagaccctgaacgagctggaggagatcagacccgagatcttcagactggtggagcaaggcttcggcatctgcagattcggcagcctgagcctgagcagcttcgaggccatcagagccgccaaggccgtgatctacggctacttcagcaccgccctgaagcacgaccacaacacccccatcacagatgacgagagacaagccagagaccccgagctgttcggcctgctgaacaagctgagaaccaggagacagaacaagggcgaggagaagatcaacagagccgctaacgccctgatcagaatcgccctggagaacaaggccgacttcatcagaggcgagggcgacctgcccaccaccaacaagagcgtcaagaaggggcagaacagcagaagcatggactggctggctagaggcctgttcaacaagatctgtcagctggccgtgatgcacaacatcattcccaccgccgccaacccccacagcacaagccaccaagaccccttcgagcacaacaaggacttcgacgaccctcagcccgccatgaagtgcagatacgctgagttcaagatcgaggacatcggcgacagcgtgctggagagactgggcacctccctgagaaatgccaaggccgggcagaccggcgcctactattacaagggcgcccaagacttcctggcccactacggcctgcaagacatcgaggaagagctgaagaagtgcagaagaggcagaaaggccaacatgccctgctgggagctgcagaagagactgattcagaagctgggcagcaaggacgccgtggtgtacatccccatgagaggcggccggatctacctggctacacacagcgtgaccaccggcgccaagcccatcatcttcaacggcgaagaggtgtgggtgagcaacgccgacgagatcgccgccgtgaacatcggcctgaccatcatccccacaagcaagaaggacgaggagcaccccgacagagagaacgacgacaaggccatcagaaagagcgtcagacggcctagaaagagcggcgagagaagaagcggcgacaagatgcccgccaccgctagaaagaccacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagggtggaggaggtgggtctggcgtggaggacgacagccgcctgctgctgggcatctacgagcacggctacggcaactgggagctgatcaagaccgaccccgagctgaagctgaccgacaagctggagtacccatacgatgttccagattacgcttgagaattccccttgagcatctgacttctggctaataaaggaaatttattttcattgcaatagtgtgttggaattttttgtgtctctcaggtaccgaaaaacatcattgcgtcgcgaggtgaggcgtcaatgggagacgcattacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagttaacggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtccgtcga

(19) United States
(12) Patent Application Publication
(10) Pub. No.: US 2026/0308641 A1
(43) Pub. Date: Oct. 8, 2026

(54) CAS12 PROTEINS AND USES THEREOF

(71) Applicants: REFORGENE MEDICINE, Guangzhou (CN); ZHEJIANG SYNSORBIO TECHNOLOGY CO., LTD., Shaoxing (CN)

(72) Inventors: Junbin LIANG, Guangzhou (CN); Liancheng HUANG, Guangzhou (CN); Chongjian CHEN, Shaoxing (CN); Yang SUN, Shaoxing (CN); Weiye PAN, Shaoxing (CN); Hui XU, Guangzhou (CN); Kaiwei SI, Guangzhou (CN); Jinxiu CAI, Guangzhou (CN); Qing LIAO, Guangzhou (CN); Desheng HUANGFU, Guangzhou (CN)

(73) Assignees: REFORGENE MEDICINE, Guangzhou (CN); ZHEJIANG SYNSORBIO TECHNOLOGY CO., LTD., Shaoxing (CN)

(21) Appl. No.: 19/717,620

(22) Filed: Jun. 23, 2026

Related U.S. Application Data
(63) Continuation of application No. 19/303,600, filed on Aug. 19, 2025.
Application No. 19/303,600 is a continuation-in-part of application No. PCT/CN2024/119862, filed on Sep. 19, 2024.

(30) Foreign Application Priority Data
Sep. 19, 2023 (CN) 202311214330.6
Apr. 1, 2024 (CN) 202410388592.2

(51) Int. Cl. C12N 15/90; C12N 9/22; C12N 15/10; C12N 15/11

(52) U.S. Cl. CPC C12N 15/907; C12N 9/226; C12N 15/1082; C12N 15/11; C12N 15/90; C12N 2310/20

(57) ABSTRACT

Cas12 protein, guide polynucleotide, inactivated Cas12 mutant, fusion protein or conjugate including the Cas12 protein, isolated nucleic acid, CRISPR-Cas12 system, vector system, delivery system, cell, pharmaceutical composition, and kit, and the use thereof are provided.

### CROSS REFERENCE TO RELATED APPLICATIONS

This application is a continuation application of U.S. application Ser. No. 19/303,600, filed on Aug. 19, 2025, which is a continuation-in-part application of International Application No. PCT/CN2024/119862, filed on Sep. 19, 2024, which claims priority to Chinese Patent Application No. 202311214330.6, filed on Sep. 19, 2023, and Chinese Patent Application No. 202410388592.2, filled on Apr. 1, 2024, the entire contents of each of which are incorporated herein by reference.

### SEQUENCE LISTING

The instant application contains a Sequence Listing which has been submitted electronically in XML format and is hereby incorporated by reference in its entirety. The XML copy, created on Sep. 28, 2025, is named “2025 Sep. 28-Sequence Listing-20954-0003US00”, and is 1,060,505 bytes in size.

### TECHNICAL FIELD

The present disclosure relates to the field of CRISPR gene editing, and, more particularly, to Cas12 proteins and uses thereof.

### BACKGROUND

A CRISPR-Cas system is an adaptive immune defense developed by bacteria and archaea over a long period of time, which is used to fight against invading viruses and exogenous DNA. The clustered regularly interspaced short palindromic repeat (CRISPR) and the CRISPR-associated protein system (CRISPR-Cas system) can be used to make changes to gene sequences directly in cells, which is a fast and effective manner.

Many researchers in this field are working on finding new Cas12 proteins and CRISPR-Cas12 gene editing systems.

### SUMMARY

The present disclosure provides Cas12 proteins and uses thereof.

Embodiments of the present disclosure provide a Cas12 protein. In some embodiments, the Cas12 protein is selected from the group consisting of a CLUSTER1 protein, a CLUSTER2 protein, a CLUSTER3 protein, a CLUSTER4 protein, a CLUSTER5 protein, a CLUSTER6 protein, a CLUSTER7 protein, a CLUSTER8 protein, a CLUSTER9 protein, a CLUSTER10 protein, a CLUSTER11 protein, a CLUSTER12 protein, and a CLUSTER13 protein. As used herein, a CLUSTER refers to a Cas12 protein family classified according to a phylogenetic analysis as shown in FIG. 1A.

In some embodiments, the Cas12 protein comprises an amino acid sequence having at least 50% sequence identity to any one of the amino acid sequences shown in SEQ ID NO: 1-53, SEQ ID NO: 696, or SEQ ID NO: 728.

In some embodiments, the Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to any one of the amino acid sequences shown in SEQ ID NO: 1-53, SEQ ID NO: 696, or SEQ ID NO: 728.

In some embodiments, the Cas12 protein comprises an amino acid sequence having at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to any one of the amino acid sequences shown in SEQ ID NO: 1-53, SEQ ID NO: 696, or SEQ ID NO: 728.

In some embodiments, the Cas12 protein comprises an amino acid sequence having at least 80% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 85% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 90% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 95% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 97% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 98% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 99% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 99.5% sequence identity to any one of SEQ ID NO: 1-53, SEQ ID NO: 696, or SEQ ID NO: 728. In some embodiments, the Cas12 protein comprises an amino acid sequence having at least 99.7% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 99.8% sequence identity to any one of the amino acid sequences shown in SEQ ID NO: 1-53, SEQ ID NO: 696, or SEQ ID NO: 728. In some embodiments, the Cas12 protein comprises an amino acid sequence having at least 100% sequence identity to any one of the amino acid sequences shown in SEQ ID NO: 1-53, SEQ ID NO: 696, or SEQ ID NO: 728.

In some embodiments, the Cas12 protein retains a function of a protein having an amino acid sequence as shown in any one of SEQ ID NO: 1-53, SEQ ID NO: 696, or SEQ ID NO: 728.

In some embodiments, the Cas12 protein forms a complex with a guide polynucleotide. In some embodiments, the Cas12 protein and the guide polynucleotide specifically bind to a target nucleic acid.

In some embodiments, the Cas12 protein forms a complex with a guide polynucleotide, and the complex specifically binds to a target nucleic acid. In some embodiments, the Cas12 protein forms a complex with a guide polynucleotide, and the complex specifically binds to a target DNA.

In some embodiments, the Cas12 protein and a guide polynucleotide specifically binds to and cleaves a target nucleic acid. In some embodiments, the Cas12 protein and a guide polynucleotide specifically binds to and cleaves a target DNA. In some embodiments, the Cas12 protein forms a complex with a guide polynucleotide, and the complex specifically binds and cleaves a target nucleic acid. In some embodiments, the Cas12 protein forms a complex with the guide polynucleotide, and the complex specifically binds and cleaves the target DNA.

As used herein, the phrase “retaining a function of a protein having an amino acid sequence as shown in any one of SEQ ID NO: 1-53, SEQ ID NO: 696, or SEQ ID NO: 728” refers to retaining the ability to form a complex with a guide polynucleotide, retaining the ability to bind a target nucleic acid complementary to the guide sequence, retaining the ability to specifically cleave the target nucleic acid with the guide polynucleotide, and/or retaining the ability to process an RNA transcript containing the guide sequence into guide polynucleotide molecules.

In some embodiments, the retaining a function of a protein having an amino acid sequence as shown in any one of SEQ ID NO: 1-53, SEQ ID NO: 696, or SEQ ID NO: 728 refers to retaining the ability to form a complex with a guide polynucleotide.

In some embodiments, the retaining a function of a protein having an amino acid sequence as shown in any one of SEQ ID NO: 1-53, SEQ ID NO: 696, or SEQ ID NO: 728 refers to retaining the ability to bind a target nucleic acid complementary to the guide sequence of the guide polynucleotide.

In some embodiments, the retaining a function of a protein having an amino acid sequence as shown in any one of SEQ ID NO: 1-53, SEQ ID NO: 696, or SEQ ID NO: 728 refers to retaining the ability to specifically cleave a target nucleic acid with a guide polynucleotide.

In some embodiments, the retaining a function of a protein having an amino acid sequence as shown in any one of SEQ ID NO: 1-53, SEQ ID NO: 696, or SEQ ID NO: 728 refers to retaining the ability to process the RNA transcript containing the guide sequence into guide polynucleotide molecules.

In some embodiments, the Cas12 protein comprises an amino acid sequence as shown in any one of SEQ ID NO: 1-53, SEQ ID NO: 696, or SEQ ID NO: 728.

In some embodiments, a protospacer adjacent motif (PAM) sequence (5′→3′) recognized by the Cas12 protein is selected from any one or more of the following:
A, C, T, G,
TA, TC, GN, AA, AG, TG, AN, GG, CG, TN, NT, NG, GT, NA, CC, AC, GC, AT, CT, GA, TT, CN, NC, CA,
NTN, ANN, TTN, ATC, NAC, AGA, TGC, TCT, NGN, CGC, NTC, GCA, TCG, TTT, CCG, GGG, NAG, ACA, CGG, CNG, ACN, GTG, CNT, TTG, TCN, GGT, TNC, CCN, CGT, TGG, CGA, NGG, TCC, AGT, NCA, CAN, TCA, NNG, TAC, CCT, NTG, CGN, TGN, CAT, NGC, GNG, GNC, NNA, GAA, TTC, CTT, ATA, TAT, GCT, NCC, TTA, AGN, GNN, CAA, CAC, AGG, NTT, ANG, GNA, GTT, NGA, TAA, GTA, GGN, GNT, NCG, ATT, CCA, CNN, AAA, AAC, ATN, GAG, CTG, ACG, NAA, TAN, NAT, CNA, GCN, GTC, NCN, CTN, CNC, ANT, NNC, CAG, NAN, ATG, NCT, CCC, AAN, TGT, TNA, ACC, GAT, ACT, AAT, GGA, GAN, ANC, GAC, NNT, CTA, TNN, GCG, GTN, TNT, AAG, TAG, NGT, NTA, ANA, CTC, GCC, TGA, GGC, AGC, TNG,
NGAA, GANC, GCNC, NTNT, TGGG, AAGG, AAGN, NTNN, TCGT, CNTG, NTGG, CCGN, ATAT, TGCA, NGGT, TGNT, NNTG, NCCG, ACAT, GNTG, CGCG, GACN, NTCG, TCNG, CTGC, TNNC, GGTN, CGNN, TCCA, AGCN, TNAG, GGAC, GATC, AANA, NATG, CCAG, NAAT, TCNT, CACT, CGGC, CGAN, CNCA, ATNT, NNNG, NGCT, CTGG, GGAN, NTNC, ATTC, AATG, CNTC, TGGN, NATC, GTCG, ACNC, GCNN, GACT, CTNT, NCTT, NAGG, NANC, CTTA, GTCT, ANAG, NGCN, CNNA, TCAG, ACAC, NCGG, TNNT, CAAG, ACCT, CCCA, GTNC, ANTC, GACC, AACG, TTAA, TCCG, CGCC, NCCN, TTNA, NCNT, NGCA, AGNN, AATC, GGGA, GNAN, NAGA, CGNA, GTAT, GTNA, ATNC, ACNA, GGAA, NTCC, GGCG, AATN, CNNT, AGGC, GCGN, GTGC, TTGA, AAGC, GAAG, ATNG, TGCT, TACT, CTAN, GGCT, GNGC, GTCN, CGAA, CNAC, GCCT, TAGG, ANGC, TNAA, GANT, NCNA, NCCT, AGAN, GTAA, TTTN, ATGA, TGNA, CANC, ACGA, CCAC, CCGG, CTNG, CNGN, GGTA, NGNC, GTTT, CTAA, TNCT, CTGN, NGAC, TGTA, TANN, GCNT, GCTC, CNCG, AAAN, CCNT, GANA, CACA, CTNA, ANTN, TINT, CCTG, TNTT, CANA, NTAN, CACG, GGAT, TTTC, GNCG, TACA, GTAC, GAGC, ACNN, ATGG, AANT, ATCC, ACCG, AGNC, TGTT, NCAT, ATTA, GNTT, GAGN, TNAC, GCCG, NTNG, GTGG, GNGN, ACCA, NTAA, ACTN, NCTG, NCTA, TTTT, GCNG, NTAG, CAAA, GGNA, CNTN, TTAG, TCTG, NCTN, TATG, GCGT, TANT, GGGT, NACN, ACTG, CCNG, GNNT, CCAT, GNTA, NANT, TACN, TGTN, ATCT, NCAN, TNGG, CNNN, AAGT, ATTN, GGNN, CAGC, CGTN, GCCC, GCTT, CNAT, NANA, CCNN, GNGA, TNGN, GCAG, CGNG, CCTT, NGAG, NCNG, AANG, GGTC, ACTC, TGAA, NAGN, NNCA, ACGG, TGAC, TCCN, ANNN, TCGN, TAAN, CAGG, TTAN, NGAN, NTGC, CCNC, TNTN, ATGN, GTGN, GCAT, NNGN, NNCC, CCNA, CNAG, GNAC, CGNT, TTCN, TAGN, ANCT, NATN, GTGA, TNGT, CTAT, CCCG, TNCA, NGTA, NNGA, CGTG, TAAT, CGCA, NNCG, NGTC, NAGT, GNAT, TNTC, NCGC, NGGN, CATN, GTTN, AGTA, GNNG, TTNN, TGNC, NAAA, TNCC, CACC, CTCT, TTGN, GCTA, NTTT, TGAN, TNAN, NGAT, CCTN, GAAT, GTCA, NTCN, GCCA, ANTG, TGGC, CAAC, TTTA, TGTC, CGGA, NCGN, AGNT, NCGA, ANCG, ACAA, TAGT, CGAG, NCAA, AATA, AGGG, GNGT, CAGA, AGGT, GGGG, ANAC, TGGT, GTGT, GNCA, GTTA, NGTT, TNNG, NCAG, CACN, GCAN, GAAC, NCCA, TTCC, NCNN, GNNN, ANGT, NTNA, CCCT, GNAA, TING, GINN, GGNG, TCTA, NCAC, GANG, TTCG, CCTC, CNGG, ANNA, TCAN, ATCG, NTGA, CGTA, TTAC, GCTN, GCTG, NGTG, TCCC, CANN, NNNA, TAGA, ACGT, AGAT, GATG, GCCN, TGNG, GCGC, CCGA, GNCN, NTTG, NNAT, TNCG, NANG, GGTG, NCCC, GNCC, CAAT, CGCN, CNGA, NTTC, TTCT, NGGA, AGTC, CNNC, NACG, AGTN, NANN, ACAG, GNCT, TACC, CNTA, TGTG, CATC, GACA, TCTT, NTCT, CTGA, AGGA, GATA, TNAT, CCTA, GGAG, ANCC, AANC, GTAN, GCNA, TGNN, TANC, GNTN, AGCG, CTAG, NNAA, AGTT, CTAC, TACG, TTNC, TNTA, ANTT, ATAC, TCCT, TCAC, NGGC, NTTN, NNTC, CANT, ATAA, TGCC, CTCC, TNNA, GING, ACGN, GGCA, AAAG, TTGT, NGNA, NAAN, TATN, CGGG, CATA, ATGC, ACGC, ACCN, ATTT, TCNA, TNGC, NACA, NACC, CTCN, GGCC, TANG, AGAA, TNGA, TAGC, CAGN, GGCN, ANNT, NNNC, TCAT, CATT, TAAA, ATGT, TGAG, CGCT, TCGG, GCAC, GTAG, NICA, NATT, ANTA, CCCN, ACTA, AAAA, GAAN, TATT, NNAC, TGAT, GGGN, CCAA, GNGG, CCAN, GTCC, NNCT, AGNG, CNTT, CNCT, GANN, GGTT, AGCT, CATG, NTAC, TNCN, NNTN, TGGA, GATT, AGCA, TAAG, GCGA, ACTT, ANGN, NTGN, AACN, AACT, TCAA, NTAT, TCGA, NCTC, NNGG, ANGG, NNTT, GTNT, CTNN, CGGN, TAAC, GGNC, GAAA, ACNG, GNAG, TTGG, CTTC, CNGT, TNNN, TNTG, GTTG, TCNN, CGGT, GAGA, CNNG, NCNC, GAGG, AGCC, ATNN, NNNT, AGAC, AACC, ANNC, ANNG, ACAN, GTTC, TATA, GNTC, NCGT, NGNT, CGTC, CCGC, CGAC, GACG, ATTG, GNNC, CNAA, TATC, AGNA, CTNC, TTCA, ANCA, ACCC, AGTG, CCGT, ANAT, CTGT, GGGC, NTTA, NAAG, AANN, CNAN, NNCN, ANAA, ANAN, CTTG, NGNN, AGAG, TANA, TCNC, GCAA, NGNG, NAGC, NATA, ATCN, CGTT, CNGC, GATN, NNTA, AAGA, CTTT, AAAC, AGGN, ACNT, NTGT, CTTN, ATCA, NACT, NNAG, NGTN, NAAC, TGCG, GGNT, ATAN, TTGC, ANCN, CCCC, ANGA, NGCG, TCTC, CTCG, ATNA, AATT, NNAN, NNGT, TCGC, ATAG, CAAN, AACA, TTAT, CAGT, GNNA, TGCN, GCGG, NGGG, CANG, TTTG, GAGT, AAAT, CTCA, CNCN, CNCC, TCTN, CGNC, NGCC, CGAT, NNGC.

N is A, T, C, or G.

In some embodiments, a PAM sequence recognized by the Cas12 protein is 5′-T-3′.

In some embodiments, a PAM sequence recognized by the Cas12 protein is 5′-G-3′.

In some embodiments, a PAM sequence recognized by the Cas12 protein is 5′-A-3′.

In some embodiments, a PAM sequence recognized by the Cas12 protein is 5′-C-3′.

In some embodiments, a PAM sequence recognized by the Cas12 protein is 5′-TA-3′.

In some embodiments, a PAM sequence recognized by the Cas12 protein is 5′-TC-3′.

In some embodiments, a PAM sequence recognized by the Cas12 protein is 5′-TG-3′.

In some embodiments, a PAM sequence recognized by the Cas12 protein is 5′-TT-3′.

In some embodiments, a PAM sequence recognized by the Cas12 protein is 5′-TN-3′.

In some embodiments, a PAM sequence recognized by the Cas12 protein is 5′-TTN-3′.

In some embodiments, a PAM sequence recognized by the Cas12 protein is 5′-TTT-3′.

In some embodiments, a PAM sequence recognized by the Cas12 protein is 5′-TTG-3′.

In some embodiments, a PAM sequence recognized by the Cas12 protein is 5′-TTC-3′.

In some embodiments, a PAM sequence recognized by the Cas12 protein is 5′-TTA-3′.

In some embodiments, a PAM sequence recognized by the Cas12 protein is 5′-WTN-3′.

In some embodiments, a PAM sequence recognized by the Cas12 protein is 5′-ATN-3′.

N is A, T, C, or G, and W is A or T.

In some embodiments of the present disclosure, the Cas12 protein is an inactivated Cas12 mutant. In some embodiments of the present disclosure, the Cas12 protein is a nuclease-inactivated mutant. In some embodiments of the present disclosure, the Cas12 protein is a dead Cas12 mutant or a nickase Cas12 mutant. In some embodiments, the Cas12 protein has an inactivated Ruvc domain.

In some embodiments of the present disclosure, the Cas12 protein is selected from an active fragment constituting the Cas12 protein described in the present disclosure.

In some embodiments of the present disclosure, the Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to any one of the amino acid sequences shown in SEQ ID NO: 46, SEQ ID NO: 696, SEQ ID NO: 52, or SEQ ID NO: 728.

In some embodiments, the Cas12 protein forms a complex with a guide polynucleotide. Further, the complex specifically binds to a target nucleic acid. Further, the complex cleaves the target nucleic acid, modifies the target nucleic acid, and/or modulates the expression of the target nucleic acid.

In some embodiments, the Cas12 protein forms a complex with a guide polynucleotide, and the guide polynucleotide comprises a guide sequence that is reverse complementary to a target sequence in a target nucleic acid. Further, the guide polynucleotide comprises a scaffold sequence that interacts with the Cas12 protein. Further, the scaffold sequence comprises a direct repeat (DR) sequence. Further, the DR sequence comprises a nucleotide sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of the sequences shown in SEQ ID NO: 704, SEQ ID NO: 529, or SEQ ID NO: 534.

In some embodiments, the scaffold sequence does not comprise a tracrRNA sequence.

In some embodiments, a PAM sequence recognized by the Cas12 protein is 5-TTN-3′ and/or 5′-TTNC-3′. N may be A, T, C, or G.

Some embodiments of the present disclosure provide a Cas12 protein. The Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 46.

In some embodiments, the Cas12 protein forms a complex with a guide polynucleotide. Further, the complex specifically binds to a target nucleic acid. Further, the complex cleaves the target nucleic acid, modifies the target nucleic acid, and/or modulates an expression of the target nucleic acid.

In some embodiments, the Cas12 protein forms a complex with a guide polynucleotide, and the guide polynucleotide comprises a guide sequence that is reverse complementary to a target sequence in the target nucleic acid. Further, the guide polynucleotide comprises a scaffold sequence that interacts with the Cas12 protein. Further, the scaffold sequence comprises a DR sequence. Further, the scaffold sequence comprises a sequence having at least 80%, at least 85%, at least 90%, at least 95%, at least 96%, or at least 97% sequence identity to the sequence shown in SEQ ID NO: 704.

Some embodiments of the present disclosure provide a Cas12 protein. In some embodiments, the Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 696.

In some embodiments, the Cas12 protein forms a complex with a guide polynucleotide. Further, the complex specifically binds to a target nucleic acid. Further, the complex cleaves the target nucleic acid, modifies the target nucleic acid, and/or modulates the expression of the target nucleic acid.

In some embodiments, the Cas12 protein forms a complex with a guide polynucleotide, and the guide polynucleotide comprises a guide sequence that is reverse complementary to a target sequence in a target nucleic acid. Further, the guide polynucleotide comprises a scaffold sequence that interacts with the Cas12 protein. Further, the scaffold sequence comprises a DR sequence. Further, the scaffold sequence comprises a sequence having at least 80%, at least 85%, at least 90%, at least 95%, at least 96%, or at least 97% sequence identity to the sequence shown in SEQ ID NO: 704.

Some embodiments of the present disclosure provide a Cas12 protein. The Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 52.

In some embodiments, the Cas12 protein forms a complex with a guide polynucleotide. Further, the complex specifically binds to a target nucleic acid. Further, the complex cleaves the target nucleic acid, modifies the target nucleic acid, and/or modulates the expression of the target nucleic acid.

In some embodiments, the Cas12 protein forms a complex with a guide polynucleotide, and the guide polynucleotide comprises a guide sequence that is reverse complementary to a target sequence in a target nucleic acid. Further, the guide polynucleotide comprises a scaffold sequence that interacts with the Cas12 protein. Further, the scaffold sequence comprises a DR sequence. Further, the scaffold sequence comprises a sequence having at least 80%, at least 85%, at least 90%, at least 95%, at least 96%, or at least 97% sequence identity to the sequence shown in SEQ ID NO: 534.

Some embodiments of the present disclosure provide a Cas12 protein. The Cas12 protein comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 728.

In some embodiments, the Cas12 protein forms a complex with a guide polynucleotide. Further, the complex specifically binds to a target nucleic acid. Further, the complex cleaves the target nucleic acid, modifies the target nucleic acid, and/or modulates the expression of the target nucleic acid.

In some embodiments, the Cas12 protein forms a complex with a guide polynucleotide, and the guide polynucleotide comprises a guide sequence that is reverse complementary to a target sequence in a target nucleic acid. Further, the guide polynucleotide comprises a scaffold sequence that interacts with the Cas12 protein. Further, the scaffold sequence comprises a DR sequence. Further, the scaffold sequence comprises a sequence having at least 80%, at least 85%, at least 90%, at least 95%, at least 96%, or at least 97% sequence identity to the sequence shown in SEQ ID NO: 534.

In some embodiments of the present disclosure, the Cas12 protein comprises an amino acid sequence having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 46.

The Cas12 protein forms a complex with a guide polynucleotide; and the guide polynucleotide comprises a guide sequence that is reverse complementary to a target sequence in a target nucleic acid and a DR sequence.

In some embodiments, the DR sequence comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence shown in SEQ ID NO: 704.

In some embodiments, the complex binds to the target nucleic acid under the guidance of the guide sequence.

In some embodiments, a PAM sequence recognized by the Cas12 protein is 5′-TTN-3′.

N may be A, T, C or G.

In some embodiments of the present disclosure, the Cas12 protein comprises an amino acid sequence having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 696.

The Cas12 protein may form a complex with a guide polynucleotide; and the guide polynucleotide comprises a guide sequence that is reverse complementary to a target sequence in a target nucleic acid and a DR sequence.

In some embodiments, the DR sequence comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence shown in the SEQ ID NO: 704.

In some embodiments, the complex binds to the target nucleic acid under the guidance of the guide sequence.

In some embodiments, a PAM sequence recognized by the Cas12 protein is 5′-TTN-3′ In some embodiments, a PAM sequence recognized by the Cas12 protein is 5′-ATN-3′. In some embodiments, a PAM sequence recognized by the Cas12 protein is 5′-WTN-3′.

In some embodiments of the present disclosure, the Cas12 protein comprises an amino acid sequence having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 52.

The Cas12 protein may form a complex with a guide polynucleotide; and the guide polynucleotide comprises a guide sequence that is reverse complementary to a target sequence in a target nucleic acid and a DR sequence.

In some embodiments, the DR sequence comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence shown in SEQ ID NO: 534.

In some embodiments, the complex binds to the target nucleic acid under the guidance of the guide sequence.

In some embodiments, a PAM sequence recognized by the Cas12 protein is 5′-TTN-3′.

In some embodiments of the present disclosure, the Cas12 protein comprises an amino acid sequence having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the amino acid sequence shown in SEQ ID NO: 728.

The Cas12 protein may form a complex with a guide polynucleotide; and the guide polynucleotide comprises a guide sequence that is reverse complementary to a target sequence in a target nucleic acid and a DR sequence.

In some embodiments, the DR sequence comprises a nucleotide sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence shown in SEQ ID NO: 534.

In some embodiments, the complex binds to the target nucleic acid under the guidance of the guide sequence.

In some embodiments, a PAM sequence recognized by the Cas12 protein is 5′-TTN-3′.

N may be A, T, C, or G.

In some embodiments, the reverse complementation is partially complementary or fully complementary. In some embodiments, the guide sequence hybridizes to the target nucleic acid.

In some embodiments, the Cas12 protein is a mutant of a Cas protein having an amino acid sequence shown in any one of SEQ ID NO: 1-53, 696, or 728.

In some embodiments, the Cas12 protein is an inactivated mutant of a Cas protein having an amino acid sequence shown in any one of SEQ ID NO: 1-53, 696, or 728.

In some embodiments, the Cas12 protein provided herein comprises one, two, or more mutations as compared to the Cas protein with the sequence shown in any one of SEQ ID NO: 1-53, SEQ ID NO: 696, and SEQ ID NO: 728, such as a single amino acid insertion, a single amino acid deletion, a single amino acid substitution, or combinations thereof. In some examples, compared to the Cas protein with the sequence shown in any one of SEQ ID NO: 1-53, SEQ ID NO: 696, and SEQ ID NO: 728, the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 amino acid changes (e.g., insertions, deletions, or substitutions) while retaining the ability to bind the target nucleic acid molecule complementary to the guide sequence of the guide polynucleotide, and/or retaining the ability to process an RNA transcript containing the guide sequence into the guide polynucleotide molecules. In some embodiments, compared to the Cas protein with the sequence shown in any one of SEQ ID NO: 1-53, SEQ ID NO: 696, and SEQ ID NO: 728, the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 amino acid changes (e.g., insertions, deletions, or substitutions) while retaining the ability to bind the target nucleic acid molecule complementary to the guide sequence of the guide polynucleotide.

In some embodiments of the present disclosure, the Cas12 protein comprises a mutation at any amino acid residue corresponding to the sequence shown in SEQ ID NO: 696. In some embodiments, the mutation is a mutation to any other natural amino acid residue. In some embodiments, the mutation is a mutation to residue R, H, K, or A. In some embodiments, the mutation is a mutation to residue R. In some embodiments, the mutation is a mutation to residue A. In some embodiments, the mutation is mutated to residue H. In some embodiments, the mutation is a mutation to residue K.

In some embodiments of the present disclosure, the Cas12 protein has mutations at the amino acid residues corresponding to positions 1-41, 42-195, 196-290, 291-358, 359-479, 480-636, 637-689, 690-846, 847-884, 885-959, 960-1080 or 1081-1139 of the sequence shown in SEQ ID NO: 696.

In some embodiments of the present disclosure, the Cas12 protein has the mutation in the RuvC domain corresponding to the sequence shown in SEQ ID NO: 696. In some embodiments of the present disclosure, the Cas12 protein has mutations at the amino acid residues corresponding to positions 637-689, 885-959, or 1081-1139 of the sequence shown in SEQ ID NO: 696.

In some embodiments of the present disclosure, the Cas12 protein has the mutation in a helical domain corresponding to the sequence shown in SEQ ID NO: 696. In some embodiments of the present disclosure, the Cas12 protein has the mutations at the amino acid residues corresponding to positions 42-195, 291-479, or 690-846 of the sequence as shown in SEQ ID NO: 696.

In some embodiments of the present disclosure, the Cas12 protein has the mutation at the amino acid residue corresponding to positions 1-41 of the sequence as shown in SEQ ID NO: 696. In some embodiments of the present disclosure, the Cas12 protein has the mutation at the amino acid residue corresponding to positions 42-195 of the sequence as shown in SEQ ID NO: 696. In some embodiments of the present disclosure, the Cas12 protein has the mutation at the amino acid residue corresponding to positions 196-290 of the sequence as shown in SEQ ID NO: 696. In some embodiments of the present disclosure, the Cas12 protein has the mutation at the amino acid residue corresponding to positions 291-358 of the sequence as shown in SEQ ID NO: 696. In some embodiments of the present disclosure, the Cas12 protein has the mutation at the amino acid residue corresponding to positions 359-479 of the sequence as shown in SEQ ID NO: 696. In some embodiments of the present disclosure, the Cas12 protein has the mutation at the amino acid residue corresponding to positions 480-636 of the sequence as shown in SEQ ID NO: 696. In some embodiments of the present disclosure, the Cas12 protein has the mutation at the amino acid residue corresponding to positions 637-689 of the sequence as shown in SEQ ID NO: 696. In some embodiments of the present disclosure, the Cas12 protein has the mutation at the amino acid residue corresponding to positions 690-846 of the sequence as shown in SEQ ID NO: 696. In some embodiments of the present disclosure, the Cas12 protein has the mutation at the amino acid residue corresponding to positions 847-884 of the sequence as shown in SEQ ID NO: 696. In some embodiments of the present disclosure, the Cas12 protein has the mutation at the amino acid residue corresponding to positions 885-959 of the sequence as shown in SEQ ID NO: 696. In some embodiments of the present disclosure, the Cas12 protein has the mutation at the amino acid residue corresponding to positions 960-1080 of the sequence as shown in SEQ ID NO: 696. In some embodiments of the present disclosure, the Cas12 protein has the mutation at the amino acid residue corresponding to positions 1081-1139 of the sequence as shown in SEQ ID NO: 696.

In some embodiments of the present disclosure, at the amino acid residues corresponding to positions 1-41 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 42-195 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 196-290 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 291-358 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 359-479 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 480-636 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 637-689 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 690-846 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 847-884 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 885-959 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 960-1080 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696. In some embodiments of the present disclosure, at the amino acid residues corresponding to positions 1081-1139 of the sequence as shown in SEQ ID NO: 696, the Cas12 protein has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in SEQ ID NO: 696.

In some embodiments of the present disclosure, the Cas12 protein has at least one mutation in at least 1, at least 2, at least 3, at least 4, at least 5, at least 6, at least 7, at least 8, at least 9, at least 10, at least 11, or at least 12 of the amino acid residues corresponding to positions 1, 2, 3, 4, 5, 6, 7, 8, 9, 10, 12, 13, 14, 15, 16, 19, 21, 22, 23, 24, 25, 26, 27, 28, 29, 30, 32, 33, 34, 35, 36, 37, 38, 39, 40, 41, 42, 43, 44, 46, 47, 48, 49, 50, 51, 53, 54, 55, 56, 57, 58, 59, 60, 61, 62, 64, 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, 101, 102, 103, 104, 105, 106, 108, 109, 110, 111, 112, 114, 115, 116, 117, 118, 119, 120, 121, 123, 124, 125, 126, 127, 128, 129, 130, 131, 132, 133, 134, 135, 136, 137, 138, 139, 140, 141, 142, 143, 144, 145, 146, 147, 148, 149, 151, 152, 153, 154, 155, 156, 157, 158, 159, 160, 161, 162, 163, 164, 165, 166, 167, 169, 170, 171, 172, 174, 175, 176, 177, 178, 179, 180, 182, 183, 184, 185, 186, 187, 188, 189, 190, 191, 192, 194, 195, 196, 197, 198, 199, 200, 202, 203, 204, 205, 206, 207, 208, 209, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 242, 243, 244, 245, 247, 248, 249, 250, 251, 252, 253, 255, 256, 257, 258, 259, 260, 261, 262, 263, 265, 266, 267, 268, 269, 270, 271, 272, 273, 274, 275, 276, 278, 279, 281, 282, 283, 284, 285, 286, 287, 288, 289, 290, 291, 292, 293, 294, 295, 296, 297, 298, 299, 300, 301, 302, 303, 305, 306, 308, 309, 310, 313, 315, 316, 317, 318, 319, 320, 321, 322, 323, 324, 325, 327, 328, 329, 330, 331, 332, 333, 334, 335, 336, 337, 339, 340, 341, 342, 343, 344, 345, 346, 347, 348, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 431, 432, 433, 435, 436, 437, 439, 440, 441, 442, 443, 444, 446, 447, 448, 449, 450, 451, 452, 453, 454, 455, 456, 457, 458, 459, 460, 461, 462, 463, 464, 467, 469, 470, 471, 472, 473, 474, 475, 476, 477, 478, 479, 480, 481, 482, 483, 484, 485, 486, 487, 488, 489, 490, 491, 492, 493, 494, 496, 497, 499, 500, 501, 502, 503, 504, 506, 507, 508, 509, 510, 511, 512, 513, 514, 515, 516, 517, 518, 519, 520, 521, 522, 523, 524, 525, 526, 527, 528, 529, 531, 532, 533, 534, 535, 536, 537, 538, 539, 540, 541, 542, 543, 544, 545, 546, 547, 548, 549, 550, 552, 553, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 589, 590, 592, 593, 594, 595, 596, 597, 598, 599, 601, 602, 603, 604, 605, 606, 607, 608, 609, 610, 611, 612, 613, 614, 615, 616, 618, 619, 620, 621, 622, 623, 624, 625, 626, 627, 628, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 678, 679, 680, 681, 683, 684, 685, 686, 688, 689, 691, 692, 693, 694, 695, 696, 697, 698, 699, 700, 701, 702, 703, 704, 705, 706, 707, 708, 709, 710, 711, 712, 713, 715, 716, 717, 719, 720, 721, 722, 723, 724, 725, 727, 728, 729, 730, 731, 732, 733, 734, 736, 737, 738, 739, 740, 741, 742, 743, 744, 745, 746, 747, 748, 749, 751, 752, 753, 754, 755, 756, 758, 759, 760, 761, 762, 764, 765, 766, 767, 768, 769, 771, 772, 773, 774, 775, 776, 779, 780, 781, 782, 783, 784, 785, 786, 787, 789, 790, 791, 792, 794, 795, 797, 798, 800, 801, 802, 804, 805, 806, 807, 808, 809, 810, 811, 812, 813, 814, 815, 817, 818, 819, 821, 822, 823, 824, 825, 826, 827, 828, 829, 830, 831, 832, 833, 834, 835, 836, 837, 838, 839, 840, 841, 842, 844, 845, 846, 847, 848, 849, 850, 851, 852, 853, 854, 855, 856, 857, 858, 859, 860, 862, 863, 864, 865, 866, 867, 868, 870, 872, 873, 874, 875, 876, 877, 879, 880, 881, 882, 883, 884, 885, 886, 887, 888, 890, 891, 892, 893, 894, 895, 896, 897, 898, 899, 900, 901, 902, 903, 904, 905, 906, 909, 910, 911, 912, 913, 914, 916, 917, 918, 919, 920, 921, 922, 923, 924, 925, 926, 927, 928, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 963, 964, 965, 966, 967, 968, 969, 970, 971, 972, 973, 974, 975, 976, 977, 979, 980, 981, 982, 983, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1021, 1023, 1024, 1025, 1026, 1027, 1028, 1029, 1030, 1031, 1032, 1033, 1035, 1036, 1037, 1038, 1039, 1040, 1041, 1042, 1043, 1044, 1045, 1046, 1047, 1048, 1049, 1050, 1052, 1053, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1100, 1101, 1102, 1103, 1104, 1105, 1107, 1108, 1109, 1110, 1111, 1112, 1113, 1115, 1116, 1117, 1120, 1122, 1123, 1124, 1125, 1128, 1129, 1130, 1131, 1132, 1133, 1134, and 1135 of the amino acid sequence shown in SEQ ID NO: 696. In some embodiments, the mutation is a mutation to any other natural amino acid residue. In some embodiments, the mutation is a mutation to residue R, H, K, or A. In some embodiments, the mutation is a mutation to residue R. In some embodiments, the mutation is a mutation to residue A. In some embodiments, the mutation is a mutation to residue H. In some embodiments, the mutation is a mutation to residue K.

In some embodiments of the present disclosure, the Cas12 protein has at least one mutation in at least 1, at least 2, at least 3, at least 4, at least 5, at least 6, at least 7, at least 8, at least 9, at least 10, at least 11, or at least 12 of the amino acid residues corresponding to positions 1, 2, 3, 4, 5, 7, 10, 24, 30, 48, 51, 55, 58, 59, 66, 108, 118, 138, 141, 175, 178, 185, 186, 257, 333, 352, 356, 375, 376, 378, 379, 383, 397, 400, 416, 426, 443, 449, 456, 459, 462, 469, 484, 485, 509, 561, 597 607, 609, 623, 638, 639, 640, 697, 722, 731, 733, 755, 758, 771, 773, 779, 781, 784, 785, 786, 789, 792, 794, 798, 822, 823, 825, 826, 829, 830, 833, 834, 836, 842, 845, 846, 847, 850, 851, 853, 855, 856, 858, 859, 860, 866, 884, 892, 893, 900, 904, 926, 956, 985, 988, 989, 992, 993, 996, 1016, 1033, 1045, 1050, 1073, 1074, 1095, 1100, 1124, 1129, and 1132 of the amino acid sequence shown in SEQ ID NO: 696. In some embodiments, the mutation is a mutation to residue R, H, or K. In some embodiments, the mutation is a mutation to residue R.

In some embodiments of the present disclosure, the Cas12 protein has at least one mutation in at least 1, at least 2, at least 3, at least 4, at least 5, at least 6, at least 7, at least 8, at least 9, at least 10, at least 11, or at least 12 of the amino acid residues corresponding to positions 12, 29, 35, 36, 40, 53, 57, 60, 64, 71, 72, 73, 75, 94, 95, 96, 97, 99, 137, 148, 149, 153, 164, 167, 171, 172, 174, 177, 190, 192, 194, 199, 204, 207, 208, 211, 215, 228, 232, 236, 238, 244, 248, 253, 256, 258, 261, 262, 275, 282, 286, 292, 298, 300, 302, 320, 324, 328, 332, 336, 339, 366, 373, 374, 384, 389, 393, 395, 415, 432, 436, 440, 453, 458, 460, 471, 472, 474, 506, 508, 510, 519, 523, 526, 528, 531, 534, 544, 550, 570, 571, 573, 592, 594, 596, 613, 615, 616, 619, 622, 624, 626, 648, 650, 651, 652, 658, 661, 663, 684, 686, 689, 693, 704, 744, 783, 787, 800, 849, 854, 876, 888, 890, 891, 894, 912, 940, 941, 942, 945, 946, 948, 961, 971, 979, 987, 998, 1000, 1002, 1006, 1013, 1015, 1035, 1042, 1048, 1049, 1052, 1053, 1057, 1058, 1063, 1079, 1081, 1082, 1083, 1084, 1085, 1086, 1087, 1088, 1089, 1090, and 1091 of the amino acid sequence shown in SEQ ID NO: 696. In some embodiments, the mutation is a mutation to residue A.

In some embodiments of the present disclosure, the Cas12 protein has the mutations at the amino acid residues corresponding to any 1, any 2, any 3, any 4, any 5, any 6, any 7, any 8, any 9, any 10, any 11, any 12, any 13, any 14, any 15, any 16, or more positions in the amino acid sequence shown in SEQ ID NO: 696, and the positions are selected from I33, G184, S185, Q186, G194, N195, G196, G197, N245, G256, L260, Y278, S285, Y316, H350, D352, A355, A356, C385, P386, H387, G390, K391, N392, D429, Q461, Q462, Q469, E485, S491, K521, P525, L611, K629, K631, N633, D841, N898, K987, A988, G989, Q990, T991, D1010, E1013, A1136, K1138, and T1139.

In some embodiments of the present disclosure, the Cas12 protein has any 1, any 2, any 3, any 4, any 5, any 6, any 7, any 8, any 9, any 10, any 11, any 12, any 13, any 14, any 15, any 16, or more amino acid mutations of the amino acid sequence shown in SEQ ID NO: 696 at positions corresponding to the amino acid sequence shown in SEQ ID NO: 696, and the amino acid mutations are selected from I33R, G184R, S185R, Q186R, G194R, N195R, G196R, G197R, N245R, G256R, L260R, Y278R, S285R, Y316R, H350R, D352R, A355R, A356R, C385R, P386R, H387R, G390R, K391R, N392R, D429R, Q461R, Q462R, Q469R, E485R, S491R, K521R, P525R, L611R, K629R, K631R, N633R, D841R, N898R, K987R, A988R, G989R, Q990R, T991R, D1010R, E1013R, A1136R, K1138R, and T1139R.

In some embodiments of the present disclosure, the Cas12 protein has mutations at amino acid residues corresponding to a combination of positions of SEQ ID NO: 696, wherein the combination of positions is selected from the group consisting of 186+352+1+426+846+858+860, 186+352+1+426+846+860, 186+352+1+5+426+858+860, 186+352+1+3+426+858+860, 186+352+3+426+860, 186+352+1+333+426+858+860, 186+352+1+426+485+858+860, 186+352+1+5+426+860, 186+352+3+426+858+860, 186+352+5+426+858+860, 186+352+333+426+858+860, 186+352+333+426+860, 186+352+426+846+860, 186+352+5+426+860, 186+352+1+3+426+860, 5+426+860, 186+352+426+846+858+860, 186+352+1+426+485+860, 426+858+860, 7+426+858, 186+352+426+860, 186+352+426+485+858+860, 186+352+426+485+860, 426+846+858+860, 7+426+846, 186+352+860, 184+186+352+376+1132, 5+333+426, 5+426+858, 186+352+1+333+426+860, 184+186+352+3+107+426, 186+352+5+426, 333+376+426, 186+352+5, 2+5+846+858, 186+352+3+426, 5+846+858, 186+352+426+846, 186+352+7, 186+352+3+376+426, 186+352+376+426+860+865, 186+352+376+426, 3+846+860, 846+858+988, 3+426+858, 3+846+858+860, 3+858+860, 186+352+858, 184+186+352+860, 333+426, 184+186+352+3+376, 3+426+860, 846+860+988, 3+860, 184+186+352+3+639, 186+352+333+426, 846+858+860, 186+352+426, 333+426+846, 333+426+485, 5+846+860, 3+846+988, 184+186+352+376, 3+333+426, 186+352+426+485, 333+426+858, 333+426+1132, 428+485, 186+352+333+376+426, 858+860, 186+352+376+426+485+860, 184+186+352+426, 186+352+639, 5+858+988, 3+858+988, 5+858, 3+858, 184+186+352+846, 184+186+352+639, 858+988, 184+186+352+426+1132, 186+352+1132, 184+186+352+5, 184+186+352+858, 858+860+1132, 3+5, 426+649, 186+352+426+485+860, 186+352+333, 184+186+352, 186+376, 846+860, 858+988+1132, 846+858, 333+376, 376+426, 184+186+352+3, 3+846+1132, 5+846+1132, 186+352+426+1132, 376+426+485+660, 426, 5+846, 846+860+1132, 333+376+485, 184+186+352+639+1132, 352+426, 333+485, 184+186+352+333, 846+858+1132, 333+426+860, 186+352+988, 5+860, 846+988, 186+352, 3+846, 846+1132, 184+186+352+1132, 186+485, 988+1132, 184+186+352+485, 376+485, 5+1132, 3+7, 186+352+485, 184+186+352+7, 184+186+352+333+336, 3+1132, 426+858+988, 186+352+376, 186+352+3, and 186+352+333+336+376+426 (mutation combinations are separated by commas). In some embodiments, the mutation is a mutation to residue R or A.

In some embodiments of the present disclosure, the Cas12 protein has mutations at amino acid residues corresponding to a combination of positions of SEQ ID NO: 696, wherein the combination of positions is selected from the group consisting of 186+352+1+426+846+858+860, 186+352+1+426+846+860, 186+352+1+5+426+858+860, 186+352+1+3+426+858+860, 186+352+3+426+860, 186+352+1+333+426+858+860, 186+352+1+426+485+858+860, 186+352+1+5+426+860, 186+352+3+426+858+860, 186+352+5+426+858+860, 186+352+333+426+858+860, 186+352+333+426+860, 186+352+426+846+860, 186+352+5+426+860, 186+352+1+3+426+860, 5+426+860, 186+352+426+846+858+860, 186+352+1+426+485+860, 426+858+860, 7+426+858, 186+352+426+860, 186+352+426+485+858+860, 186+352+426+485+860, 426+846+858+860, 7+426+846, and 186+352+860 (mutation combinations are separated by commas). In some embodiments, the mutation is a mutation to residue R or A.

In some embodiments of the present disclosure, the Cas12 protein comprises a combination of amino acid substitutions selected from the group consisting of D352R+Q186R, D352R+L260R, D352R+A355R, A355R+L260R, P386R+C385R, E485R+Q462R, G184R+Q186R, D352R+Q186R+I33R, D352R+Q186R+G184R, D352R+Q186R+S185R, D352R+Q186R+Y278R, D352R+Q186R+S285R, D352R+Q186R+G256R, D352R+Q186R+Y316R, D352R+Q186R+H350R, D352R+Q186R+A356R, D352R+Q186R+Q469R, D352R+Q186R+S491R, D352R+Q186R+K521R, D352R+Q186R+P525R, D352R+Q186R+K629R, D352R+Q186R+N633R, D352R+Q186R+D841R, D352R+Q186R+N898R, D352R+Q186R+K987R, D352R+Q186R+T991R, D352R+Q186R+D1010R, and D352R+Q186R+E1013R, wherein the amino acid positions are numbered with reference to SEQ ID NO:696.

In some embodiments of the present disclosure, the Cas12 protein comprises a mutation at an amino acid residue corresponding to position 1 of the sequence as shown in SEQ ID NO: 696. In some embodiments of the present disclosure, the Cas12 protein comprises a mutation at an amino acid residue corresponding to position 2 of the sequence as shown in SEQ ID NO: 696. In some embodiments of the present disclosure, the Cas12 protein comprises a mutation at an amino acid residue corresponding to position 3 of the sequence as shown in SEQ ID NO: 696. In some embodiments of the present disclosure, the Cas12 protein comprises a mutation at an amino acid residue corresponding to position 4 of the sequence as shown in SEQ ID NO: 696. In some embodiments of the present disclosure, the Cas12 protein comprises a mutation at an amino acid residue corresponding to position 5 of the sequence as shown in SEQ ID NO: 696. In some embodiments of the present disclosure, the Cas12 protein comprises a mutation at an amino acid residue corresponding to position 7 of the sequence as shown in SEQ ID NO: 696. In some embodiments of the present disclosure, the Cas12 protein comprises a mutation at an amino acid residue corresponding to position 10 of the sequence as shown in SEQ ID NO: 696. In some embodiments of the present disclosure, the Cas12 protein comprises a mutation at an amino acid residue corresponding to position 24 of the sequence as shown in SEQ ID NO: 696. In some embodiments of the present disclosure, the Cas12 protein comprises a mutation at amino acid residue corresponding to position 30 of the as shown in SEQ ID NO: 696. In some embodiments of the present disclosure, the Cas12 protein comprises a mutation at an amino acid residue corresponding to position 48 of the sequence as shown in SEQ ID NO: 696. In some embodiments of the present disclosure, the Cas12 protein comprises a mutation at an amino acid residue corresponding to position 51 of the sequence as shown in SEQ ID NO: 696. In some embodiments of the present disclosure, the Cas12 protein comprises a mutation at an amino acid residue corresponding to position 55 of the sequence as shown in SEQ ID NO: 696. In some embodiments of the present disclosure, the Cas12 protein comprises a mutation at an amino acid residue corresponding to position 58 of the sequence as shown in SEQ ID NO: 696. In some embodiments of the present disclosure, the Cas12 protein comprises a mutation at an amino acid residue corresponding to position 59 of the sequence as shown in SEQ ID NO: 696. In some embodiments of the present disclosure, the Cas12 protein comprises a mutation at an amino acid residue corresponding to position 66 of the sequence as shown in SEQ ID NO: 696. In some embodiments of the present disclosure, the Cas12 protein comprises a mutation at amino acid residue corresponding to position 108 of the sequence as shown in SEQ ID NO: 696. In some embodiments of the present disclosure, the Cas12 protein comprises a mutation at an amino acid residue corresponding to position 118 of the sequence as shown in SEQ ID NO: 696. In some embodiments of the present disclosure, the Cas12 protein comprises a mutation at an amino acid residue corresponding to position 138 of the sequence as shown in SEQ ID NO: 696. In some embodiments of the present disclosure, the Cas12 protein comprises a mutation at an amino acid residue corresponding to position 141 of the sequence as shown in SEQ ID NO: 696. In some embodiments of the present disclosure, the Cas12 protein comprises a mutation at an amino acid residue corresponding to position 175 of the sequence as shown in SEQ ID NO: 696. In some embodiments of the present disclosure, the Cas12 protein comprises a mutation at an amino acid residue corresponding to position 178 of the sequence as shown in SEQ ID NO: 696. In some embodiments of the present disclosure, the Cas12 protein comprises a mutation at an amino acid residue corresponding to position 185 of the sequence as shown in SEQ ID NO: 696. In some embodiments of the present disclosure, the Cas12 protein comprises a mutation at an amino acid residue corresponding to position 186 of the sequence as shown in SEQ ID NO: 696. In some embodiments of the present disclosure, the Cas12 protein comprises a mutation at an amino acid residue corresponding to position 257 of the sequence as shown in SEQ ID NO: 696. In some embodiments of the present disclosure, the Cas12 protein comprises a mutation at an amino acid residue corresponding to position 333 of the sequence as shown in SEQ ID NO: 696. In some embodiments of the present disclosure, the Cas12 protein comprises a mutation at an amino acid residue corresponding to position 352 of the sequence as shown in SEQ ID NO: 696. In some embodiments of the present disclosure, the Cas12 protein comprises a mutation at an amino acid residue corresponding to position 356 of the sequence as shown in SEQ ID NO: 696. In some embodiments of the present disclosure, the Cas12 protein comprises a mutation at an amino acid residue corresponding to position 375 of the sequence as shown in SEQ ID NO: 696. In some embodiments of the present disclosure, the Cas12 protein comprises a mutation at an amino acid residue corresponding to position 376 of the sequence as shown in SEQ ID NO: 696. In some embodiments of the present disclosure, the Cas12 protein comprises a mutation at an amino acid residue corresponding to position 378 of the sequence as shown in SEQ ID NO: 696. In some embodiments of the present disclosure, the Cas12 protein comprises a mutation at an amino acid residue corresponding to position 379 of the sequence as shown in SEQ ID NO: 696. In some embodiments of the present disclosure, the Cas12 protein comprises a mutation at an amino acid residue corresponding to position 383 of the sequence as shown in SEQ ID NO: 696. In some embodiments of the present disclosure, the Cas12 protein comprises a mutation at an amino acid residue corresponding to position 397 of the sequence as shown in SEQ ID NO: 696. In some embodiments of the present disclosure, the Cas12 protein comprises a mutation at an amino acid residue corresponding to position 400 of the sequence as shown in SEQ ID NO: 696. In some embodiments of the present disclosure, the Cas12 protein comprises a mutation at an amino acid residue corresponding to position 416 of the sequence as shown in SEQ ID NO: 696. In some embodiments of the present disclosure, the Cas12 protein comprises a mutation at an amino acid residue corresponding to position 426 of the sequence as shown in SEQ ID NO: 696. In some embodiments of the present disclosure, the Cas12 protein comprises a mutation at an amino acid residue corresponding to position 443 of the sequence as shown in SEQ ID NO: 696. In some embodiments of the present disclosure, the Cas12 protein comprises a mutation at an amino acid residue corresponding to position 449 of the sequence as shown in SEQ ID NO: 696. In some embodiments of the present disclosure, the Cas12 protein comprises a mutation at an amino acid residue corresponding to position 456 of the sequence as shown in SEQ ID NO: 696. In some embodiments of the present disclosure, the Cas12 protein comprises a mutation at an amino acid residue corresponding to position 459 of the sequence as shown in SEQ ID NO: 696. In some embodiments of the present disclosure, the Cas12 protein comprises a mutation at an amino acid residue corresponding to position 462 of the sequence as shown in SEQ ID NO: 696. In some embodiments of the present disclosure, the Cas12 protein comprises a mutation at an amino acid residue corresponding to position 469 of the sequence as shown in SEQ ID NO: 696. In some embodiments of the present disclosure, the Cas12 protein comprises a mutation at an amino acid residue corresponding to position 484 of the sequence as shown in SEQ ID NO: 696. In some embodiments of the present disclosure, the Cas12 protein comprises a mutation at an amino acid residue corresponding to position 485 of the sequence as shown in SEQ ID NO: 696. In some embodiments of the present disclosure, the Cas12 protein comprises a mutation at an acid residue corresponding to position 509 of the sequence as shown in SEQ ID NO: 696. In some embodiments of the present disclosure, the Cas12 protein comprises a mutation at an amino acid residue corresponding to position 561 of the sequence as shown in SEQ ID NO: 696. In some embodiments of the present disclosure, the Cas12 protein comprises a mutation at an amino acid residue corresponding to position 597 of the sequence as shown in SEQ ID NO: 696. In some embodiments of the present disclosure, the Cas12 protein comprises a mutation at an amino acid residue corresponding to position 607 of the sequence as shown in SEQ ID NO: 696. In some embodiments of the present disclosure, the Cas12 protein comprises a mutation at an amino acid residue corresponding to position 609 of the sequence as shown in SEQ ID NO: 696. In some embodiments of the present disclosure, the Cas12 protein comprises a mutation at an amino acid residue corresponding to position 623 of the sequence as shown in SEQ ID NO: 696. In some embodiments of the present disclosure, the Cas12 protein comprises a mutation at an amino acid residue corresponding to position 638 of the sequence as shown in SEQ ID NO: 696. In some embodiments of the present disclosure, the Cas12 protein comprises a mutation at an amino acid residue corresponding to position 639 of the sequence as shown in SEQ ID NO: 696. In some embodiments of the present disclosure, the Cas12 protein comprises a mutation at an amino acid residue corresponding to position 640 of the sequence as shown in SEQ ID NO: 696. In some embodiments of the present disclosure, the Cas12 protein comprises a mutation at an amino acid residue corresponding to position 697 of the sequence as shown in SEQ ID NO: 696. In some embodiments of the present disclosure, the Cas12 protein comprises a mutation at an amino acid residue corresponding to position 722 of the sequence as shown in SEQ ID NO: 696. In some embodiments of the present disclosure, the Cas12 protein comprises a mutation at an amino acid residue corresponding to position 731 of the sequence as shown in SEQ ID NO: 696. In some embodiments of the present disclosure, the Cas12 protein comprises a mutation at an amino acid residue corresponding to position 733 of the sequence as shown in SEQ ID NO: 696. In some embodiments of the present disclosure, the Cas12 protein comprises a mutation at an amino acid residue corresponding to position 755 of the sequence as shown in SEQ ID NO: 696. In some embodiments of the present disclosure, the Cas12 protein comprises a mutation at an amino acid residue corresponding to position 758 of the sequence as shown in SEQ ID NO: 696. In some embodiments of the present disclosure, the Cas12 protein comprises a mutation at an amino acid residue corresponding to position 771 of the sequence as shown in SEQ ID NO: 696. In some embodiments of the present disclosure, the Cas12 protein comprises a mutation at an amino acid residue corresponding to position 773 of the sequence as shown in SEQ ID NO: 696. In some embodiments of the present disclosure, the Cas12 protein comprises a mutation at an amino acid residue corresponding to position 779 of the sequence as shown in SEQ ID NO: 696. In some embodiments of the present disclosure, the Cas12 protein comprises a mutation at an amino acid residue corresponding to position 781 of the sequence as shown in SEQ ID NO: 696. In some embodiments of the present disclosure, the Cas12 protein comprises a mutation at an amino acid residue corresponding to position 784 of the sequence as shown in SEQ ID NO: 696. In some embodiments of the present disclosure, the Cas12 protein comprises a mutation at an amino acid residue corresponding to position 785 of the sequence as shown in SEQ ID NO: 696. In some embodiments of the present disclosure, the Cas12 protein comprises a mutation at an amino acid residue corresponding to position 786 of the sequence as shown in SEQ ID NO: 696. In some embodiments of the present disclosure, the Cas12 protein comprises a mutation at an amino acid residue corresponding to position 789 of the sequence as shown in SEQ ID NO: 696. In some embodiments of the present disclosure, the Cas12 protein comprises a mutation at an amino acid residue corresponding to position 792 of the sequence as shown in SEQ ID NO: 696. In some embodiments of the present disclosure, the Cas12 protein comprises a mutation at an amino acid residue corresponding to position 794 of the sequence as shown in SEQ ID NO: 696. In some embodiments of the present disclosure, the Cas12 protein comprises a mutation at an amino acid residue corresponding to position 798 of the sequence as shown in SEQ ID NO: 696. In some embodiments of the present disclosure, the Cas12 protein comprises a mutation at an amino acid residue corresponding to position 822 of the sequence as shown in SEQ ID NO: 696. In some embodiments of the present disclosure, the Cas12 protein comprises a mutation at an amino acid residue corresponding to position 823 of the sequence as shown in SEQ ID NO: 696. In some embodiments of the present disclosure, the Cas12 protein comprises a mutation at an amino acid residue corresponding to position 825 of the sequence as shown in SEQ ID NO: 696. In some embodiments of the present disclosure, the Cas12 protein comprises a mutation at an amino acid residue corresponding to position 826 of the sequence as shown in SEQ ID NO: 696. In some embodiments of the present disclosure, the Cas12 protein comprises a mutation at an amino acid residue corresponding to position 829 of the sequence as shown in SEQ ID NO: 696. In some embodiments of the present disclosure, the Cas12 protein comprises a mutation at an amino acid residue corresponding to position 830 of the sequence as shown in SEQ ID NO: 696. In some embodiments of the present disclosure, the Cas12 protein comprises a mutation at an amino acid residue corresponding to position 833 of the sequence as shown in SEQ ID NO: 696. In some embodiments of the present disclosure, the Cas12 protein comprises a mutation at an amino acid residue corresponding to position 834 of the sequence as shown in SEQ ID NO: 696. In some embodiments of the present disclosure, the Cas12 protein comprises a mutation at an amino acid residue corresponding to position 836 of the sequence as shown in SEQ ID NO: 696. In some embodiments of the present disclosure, the Cas12 protein comprises a mutation at an amino acid residue corresponding to position 842 of the sequence as shown in SEQ ID NO: 696. In some embodiments of the present disclosure, the Cas12 protein comprises a mutation at an amino acid residue corresponding to position 845 of the sequence as shown in SEQ ID NO: 696. In some embodiments of the present disclosure, the Cas12 protein comprises a mutation at an amino acid residue corresponding to position 846 of the sequence as shown in SEQ ID NO: 696. In some embodiments of the present disclosure, the Cas12 protein comprises a mutation at an amino acid residue corresponding to position 847 of the sequence as shown in SEQ ID NO: 696. In some embodiments of the present disclosure, the Cas12 protein comprises a mutation at an amino acid residue corresponding to position 850 of the sequence as shown in SEQ ID NO: 696. In some embodiments of the present disclosure, the Cas12 protein comprises a mutation at an amino acid residue corresponding to position 851 of the sequence as shown in SEQ ID NO: 696. In some embodiments of the present disclosure, the Cas12 protein comprises a mutation at an amino acid residue corresponding to position 853 of the sequence as shown in SEQ ID NO: 696. In some embodiments of the present disclosure, the Cas12 protein comprises a mutation at an amino acid residue corresponding to position 855 of the sequence as shown in SEQ ID NO: 696. In some embodiments of the present disclosure, the Cas12 protein comprises a mutation at an amino acid residue corresponding to position 856 of the sequence as shown in SEQ ID NO: 696. In some embodiments of the present disclosure, the Cas12 protein comprises a mutation at an amino acid residue corresponding to position 858 of the sequence as shown in SEQ ID NO: 696. In some embodiments of the present disclosure, the Cas12 protein comprises a mutation at an amino acid residue corresponding to position 859 of the sequence as shown in SEQ ID NO: 696. In some embodiments of the present disclosure, the Cas12 protein comprises a mutation at an amino acid residue corresponding to position 860 of the sequence as shown in SEQ ID NO: 696. In some embodiments of the present disclosure, the Cas12 protein comprises a mutation at an amino acid residue corresponding to position 866 of the sequence as shown in SEQ ID NO: 696. In some embodiments of the present disclosure, the Cas12 protein comprises a mutation at an amino acid residue corresponding to position 884 of the sequence as shown in SEQ ID NO: 696. In some embodiments of the present disclosure, the Cas12 protein comprises a mutation at an amino acid residue corresponding to position 892 of the sequence as shown in SEQ ID NO: 696. In some embodiments of the present disclosure, the Cas12 protein comprises a mutation at an amino acid residue corresponding to position 893 of the sequence as shown in SEQ ID NO: 696. In some embodiments of the present disclosure, the Cas12 protein comprises a mutation at an amino acid residue corresponding to position 900 of the sequence as shown in SEQ ID NO: 696. In some embodiments of the present disclosure, the Cas12 protein comprises a mutation at an amino acid residue corresponding to position 904 of the sequence as shown in SEQ ID NO: 696. In some embodiments of the present disclosure, the Cas12 protein comprises a mutation at an amino acid residue corresponding to position 926 of the sequence as shown in SEQ ID NO: 696. In some embodiments of the present disclosure, the Cas12 protein comprises a mutation at an amino acid residue corresponding to position 956 of the sequence as shown in SEQ ID NO: 696. In some embodiments of the present disclosure, the Cas12 protein comprises a mutation at an amino acid residue corresponding to position 985 of the sequence as shown in SEQ ID NO: 696. In some embodiments of the present disclosure, the Cas12 protein comprises a mutation at an amino acid residue corresponding to position 988 of the sequence as shown in SEQ ID NO: 696. In some embodiments of the present disclosure, the Cas12 protein comprises a mutation at an amino acid residue corresponding to position 989 of the sequence as shown in SEQ ID NO: 696. In some embodiments of the present disclosure, the Cas12 protein comprises a mutation at an amino acid residue corresponding to position 992 of the sequence as shown in SEQ ID NO: 696. In some embodiments of the present disclosure, the Cas12 protein comprises a mutation at an amino acid residue corresponding to position 993 of the sequence as shown in SEQ ID NO: 696. In some embodiments of the present disclosure, the Cas12 protein comprises a mutation at an amino acid residue corresponding to position 996 of the sequence as shown in SEQ ID NO: 696. In some embodiments of the present disclosure, the Cas12 protein comprises a mutation at an amino acid residue corresponding to position 1016 of the sequence as shown in SEQ ID NO: 696. In some embodiments of the present disclosure, the Cas12 protein comprises a mutation at an amino acid residue corresponding to position 1033 of the sequence as shown in SEQ ID NO: 696. In some embodiments of the present disclosure, the Cas12 protein comprises a mutation at an amino acid residue corresponding to position 1045 of the sequence as shown in SEQ ID NO: 696. In some embodiments of the present disclosure, the Cas12 protein comprises a mutation at an amino acid residue corresponding to position 1050 of the sequence as shown in SEQ ID NO: 696. In some embodiments of the present disclosure, the Cas12 protein comprises a mutation at an amino acid residue corresponding to position 1073 of the sequence as shown in SEQ ID NO: 696. In some embodiments of the present disclosure, the Cas12 protein comprises a mutation at an amino acid residue corresponding to position 1074 of the sequence as shown in SEQ ID NO: 696. In some embodiments of the present disclosure, the Cas12 protein comprises a mutation at an amino acid residue corresponding to position 1095 of the sequence as shown in SEQ ID NO: 696. In some embodiments of the present disclosure, the Cas12 protein comprises a mutation at an amino acid residue corresponding to position 1100 of the sequence as shown in SEQ ID NO: 696. In some embodiments of the present disclosure, the Cas12 protein comprises a mutation at an amino acid residue corresponding to position 1124 of the sequence as shown in SEQ ID NO: 696. In some embodiments of the present disclosure, the Cas12 protein comprises a mutation at an amino acid residue corresponding to position 1129 of the sequence as shown in SEQ ID NO: 696. In some embodiments of the present disclosure, the Cas12 protein comprises a mutation at an amino acid residue corresponding to position 1132 of the sequence as shown in SEQ ID NO: 696. In some embodiments, the mutation is a mutation to residue R.

In some embodiments of the present disclosure, the Cas12 protein comprises a mutation at an amino acid residue corresponding to position 651 of the sequence as shown in SEQ ID NO: 696. In some embodiments, the mutation is a mutation to residue A.

In some embodiments of the present disclosure, the Cas12 protein comprises a mutation at an amino acid residue corresponding to position 891 of the sequence as shown in SEQ ID NO: 696. In some embodiments, the mutation is a mutation to residue A.

In some embodiments of the present disclosure, the Cas12 protein comprises a mutation at an amino acid residue corresponding to position 1082 of the sequence as shown in SEQ ID NO: 696. In some embodiments, the mutation is a mutation to residue A.

In some embodiments of the present disclosure, the Cas12 protein has mutations at amino acid residues corresponding to any 1, any 2, any 3, any 4, any 5, any 6, any 7, any 8, any 9, any 10, any 11, any 12, any 13, any 14, any 15, any 16, or more positions of the amino acid sequence shown in SEQ ID NO: 52, and the positions are selected from V15, Q172, A173, G182, E183, G184, K185, K186, G239, V243, D264, E271, Y295, L297, N317, T329, E331, I335, K339, N347, E363, H366, V426, K429, S430, L433, S452, S455, S465, E493, P497, K587, E768, T825, A911, G914, K915, 1916, K918, T919, T920, A922, and E940.

In some embodiments of the present disclosure, the Cas12 protein has any 1, any 2, any 3, any 4, any 5, any 6, any 7, any 8, any 9, any 10, any 11, any 12, any 13, any 14, any 15, any 16, or more amino acid mutations of the amino acid sequence shown in SEQ ID NO: 52 at positions corresponding to the amino acid sequence shown in SEQ ID NO: 52, and the amino acid mutations are selected from V15R, Q172R, A173W, G182R, E183R, G184R, K185R, K186R, G239R, V243R, D264R, E271R, Y295R, L297R, N317R, T329R, E331R, I335R, K339R, N347E, E363R, H366R, V426R, K429R, S430R, L433R, S452R, S455R, S465R, E493R, P497R, K587R, E768R, T825R, A911R, G914R, K915R, I916R, K918R, T919R, T920R, A922R, and E940R.

In some embodiments of the present disclosure, the Cas12 protein has any 1, any 2, any 3, any 4, any 5, or more amino acid mutations of the amino acid sequence shown in SEQ ID NO: 52 at positions corresponding to the amino acid sequence shown in SEQ ID NO: 52, and the amino acid mutations are selected from N347E+K339R, Q172R+S452R, Q172R+T920R, Q172R+V426R, S452R+T920R, V426R+S452R, V426R+T920R.

In some embodiments of the present disclosure, the Cas12 protein comprises a mutation at an amino acid residue corresponding to position 15 of the sequence as shown in SEQ ID NO: 52. In some embodiments of the present disclosure, the Cas12 protein comprises a mutation at an amino acid residue corresponding to position 172 of the sequence as shown in SEQ ID NO: 52. In some embodiments of the present disclosure, the Cas12 protein comprises a mutation at an amino acid residue corresponding to position 173 of the sequence as shown in SEQ ID NO: 52. In some embodiments of the present disclosure, the Cas12 protein comprises a mutation at an amino acid residue corresponding to position 182 of the sequence as shown in SEQ ID NO: 52. In some embodiments of the present disclosure, the Cas12 protein comprises a mutation at an amino acid residue corresponding to position 183 of the sequence as shown in SEQ ID NO: 52. In some embodiments of the present disclosure, the Cas12 protein comprises a mutation at an amino acid residue corresponding to position 184 of the sequence as shown in SEQ ID NO: 52. In some embodiments of the present disclosure, the Cas12 protein comprises a mutation at an amino acid residue corresponding to position 185 of the sequence as shown in SEQ ID NO: 52. In some embodiments of the present disclosure, the Cas12 protein comprises a mutation at an amino acid residue corresponding to position 186 of the sequence as shown in SEQ ID NO: 52. In some embodiments of the present disclosure, the Cas12 protein comprises a mutation at an amino acid residue corresponding to position 239 of the sequence as shown in SEQ ID NO: 52. In some embodiments of the present disclosure, the Cas12 protein comprises a mutation at an amino acid residue corresponding to position 243 of the sequence as shown in SEQ ID NO: 52. In some embodiments of the present disclosure, the Cas12 protein comprises a mutation at an amino acid residue corresponding to position 264 of the sequence as shown in SEQ ID NO: 52. In some embodiments of the present disclosure, the Cas12 protein comprises a mutation at an amino acid residue corresponding to position 271 of the sequence as shown in SEQ ID NO: 52. In some embodiments of the present disclosure, the Cas12 protein comprises a mutation at an amino acid residue corresponding to position 295 of the sequence as shown in SEQ ID NO: 52. In some embodiments of the present disclosure, the Cas12 protein comprises a mutation at an amino acid residue corresponding to position 297 of the sequence as shown in SEQ ID NO: 52. In some embodiments of the present disclosure, the Cas12 protein comprises a mutation at an amino acid residue corresponding to position 317 of the sequence as shown in SEQ ID NO: 52. In some embodiments of the present disclosure, the Cas12 protein comprises a mutation at an amino acid residue corresponding to position 329 of the sequence as shown in SEQ ID NO: 52. In some embodiments of the present disclosure, the Cas12 protein comprises a mutation at an amino acid residue corresponding to position 331 of the sequence as shown in SEQ ID NO: 52. In some embodiments of the present disclosure, the Cas12 protein comprises a mutation at an amino acid residue corresponding to position 335 of the sequence as shown in SEQ ID NO: 52. In some embodiments of the present disclosure, the Cas12 protein comprises a mutation at an amino acid residue corresponding to position 339 of the sequence as shown in SEQ ID NO: 52. In some embodiments of the present disclosure, the Cas12 protein comprises a mutation at an amino acid residue corresponding to position 347 of the sequence as shown in SEQ ID NO: 52. In some embodiments of the present disclosure, the Cas12 protein comprises a mutation at an amino acid residue corresponding to position 363 of the sequence as shown in SEQ ID NO: 52. In some embodiments of the present disclosure, the Cas12 protein comprises a mutation at an amino acid residue corresponding to position 366 of the sequence as shown in SEQ ID NO: 52. In some embodiments of the present disclosure, the Cas12 protein comprises a mutation at an amino acid residue corresponding to position 426 of the sequence as shown in SEQ ID NO: 52. In some embodiments of the present disclosure, the Cas12 protein comprises a mutation at an amino acid residue corresponding to position 429 of the sequence as shown in SEQ ID NO: 52. In some embodiments of the present disclosure, the Cas12 protein comprises a mutation at an amino acid residue corresponding to position 430 of the sequence as shown in SEQ ID NO: 52. In some embodiments of the present disclosure, the Cas12 protein comprises a mutation at an amino acid residue corresponding to position 433 of the sequence as shown in SEQ ID NO: 52. In some embodiments of the present disclosure, the Cas12 protein comprises a mutation at an amino acid residue corresponding to position 452 of the sequence as shown in SEQ ID NO: 52. In some embodiments of the present disclosure, the Cas12 protein comprises a mutation at an amino acid residue corresponding to position 455 of the sequence as shown in SEQ ID NO: 52. In some embodiments of the present disclosure, the Cas12 protein comprises a mutation at an amino acid residue corresponding to position 465 of the sequence as shown in SEQ ID NO: 52. In some embodiments of the present disclosure, the Cas12 protein comprises a mutation at an amino acid residue corresponding to position 493 of the sequence as shown in SEQ ID NO: 52. In some embodiments of the present disclosure, the Cas12 protein comprises a mutation at an amino acid residue corresponding to position 497 of the sequence as shown in SEQ ID NO: 52. In some embodiments of the present disclosure, the Cas12 protein comprises a mutation at an amino acid residue corresponding to position 587 of the sequence as shown in SEQ ID NO: 52. In some embodiments of the present disclosure, the Cas12 protein comprises a mutation at an amino acid residue corresponding to position 768 of the sequence as shown in SEQ ID NO: 52. In some embodiments of the present disclosure, the Cas12 protein comprises a mutation at an amino acid residue corresponding to position 825 of the sequence as shown in SEQ ID NO: 52. In some embodiments of the present disclosure, the Cas12 protein comprises a mutation at an amino acid residue corresponding to position 911 of the sequence as shown in SEQ ID NO: 52. In some embodiments of the present disclosure, the Cas12 protein comprises a mutation at an amino acid residue corresponding to position 914 of the sequence as shown in SEQ ID NO: 52. In some embodiments of the present disclosure, the Cas12 protein comprises a mutation at an amino acid residue corresponding to position 915 of the sequence as shown in SEQ ID NO: 52. In some embodiments of the present disclosure, the Cas12 protein comprises a mutation at an amino acid residue corresponding to position 916 of the sequence as shown in SEQ ID NO: 52. In some embodiments of the present disclosure, the Cas12 protein comprises a mutation at an amino acid residue corresponding to position 918 of the sequence as shown in SEQ ID NO: 52. In some embodiments of the present disclosure, the Cas12 protein comprises a mutation at an amino acid residue corresponding to position 919 of the sequence as shown in SEQ ID NO: 52. In some embodiments of the present disclosure, the Cas12 protein comprises a mutation at an amino acid residue corresponding to position 920 of the sequence as shown in SEQ ID NO: 52. In some embodiments of the present disclosure, the Cas12 protein comprises a mutation at an amino acid residue corresponding to position 922 of the sequence as shown in SEQ ID NO: 52. In some embodiments of the present disclosure, the Cas12 protein comprises a mutation at an amino acid residue corresponding to position 940 of the sequence as shown in SEQ ID NO: 52. In some embodiments, the mutation is a mutation to residue R.

Embodiments of the present disclosure provides a guide polynucleotide, wherein the guide polynucleotide comprises (i) a scaffold sequence, and (ii) a guide sequence. The guide sequence has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a sequence shown in any one of SEQ ID NO: 722, SEQ ID NO: 761-782, and SEQ ID NO: 825-877. The guide polynucleotide is able to form a complex with a nucleic acid-binding polypeptide and guide a sequence-specific binding of the complex to a target nucleic acid.

In some embodiments, the guide sequence may be obtained by adding and/or deleting 1, 2, 3, 4, 5, 6, or 7 nucleotides to/from the sequence shown in any one of SEQ ID NO: 722, SEQ ID NO: 761-782, and SEQ ID NO: 825-877. In some embodiments, the guide sequence is as shown in any one of SEQ ID NO: 722, SEQ ID NO: 761-782, and SEQ ID NO: 825-877.

In some embodiments, the guide sequence hybridizes to the target nucleic acid. In some embodiments, the target nucleic acid is randomly selected from TTR, HBG, BCL11A, BACH2, KLKB1, PCSK9, SOD1, and BACE1 genes.

In some embodiments, the guide sequence is located at the 5′ end or 3′ end of the scaffold sequence.

In some embodiments, the nucleic acid-binding polypeptide and the guide polynucleotide form a complex. Further, the complex specifically binds to a target nucleic acid. Further, the complex cleaves the target nucleic acid, modifies the target nucleic acid, and/or modulates an expression of the target nucleic acid.

In some embodiments, the nucleic acid-binding polypeptide comprises a DNA-binding polypeptide. In some embodiments, the nucleic acid-binding polypeptide comprises an RNA-binding polypeptide. In some embodiments, the nucleic acid-binding polypeptide comprises a TALEN nuclease, a zinc finger nuclease, a CRISPR-Cas nuclease, or a meganuclease. In some embodiments, the nucleic acid-binding polypeptide is an RNA-guided nuclease. In some embodiments, the DNA-binding polypeptide is a CRISPR-associated nuclease, i.e., a Cas enzyme (also known as a Cas protein, a CRISPR-Cas nuclease). In some embodiments, the nucleic acid-binding polypeptide is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9, Cas10, Cas10d, Cas12a/Cpf1, Cas12b/C2c1, Cas12c/C2c3, Cas12d/CasY, Cas12e/CasX, Cas12f/CasZ, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Cas13a, Cas13b, Cas13c, Cas13d, Cas13e, Cas13f, TnpB, IscB, IsrB, and Fancor, or fragments thereof. Non-limiting examples of the fragments include nucleic acid-binding domain fragments. In some embodiments, the nucleic acid-binding polypeptide is selected from Cas9, Cas12, Cas13, TnpB, IscB, IsrB, Fancor nuclease, or fragments thereof, including but not limited to the nucleic acid-binding domain fragments. In some embodiments, the Cas9 is selected from SpCas9, SaCas9, Nme2Cas9, Nme3Cas9, CjCas9, NmCas9, FnCas9, PpnCas9, FrCas9, SauCas9, SauriCas9, ScaCas9, St1Cas9, BlatCas9, CdiCas9, GeoCas9, fragments thereof, and mutations thereof or fragments of the mutations. In some embodiments, the nucleic acid-binding polypeptide is selected from AsCpf1, enAsCas12a (addgene plasmid #196724), dFnCas12a (addgene plasmid #136379), ErCas12a, LbCas12a D832A, LbCas12a H759A, LbCas12a E795L, FnCas12a3, FnCas12a D917A, AsCas12a R1226A, AsCas12a D908A, AsCas12a E174R/S542R, AsCas12a (S542R/K548V/N552R), PrCas12a, PxCas12a, PcCas12a, PdCas12a, Mb2Cas12a, Mb3Cas12a, MlCas12a, CMaCas12a, CMtCas12a, HkCas12a, Lb5Cas12a, ErCas12a, TsCas12a, FnCpf1, LbCas12a, dLbCpf1, ttHsCas12a, AaCas12b, AaCas12b D570A, AaCas12b Q119F/E475R/E758R, BhCas12b, BvCas12b, BrCas12b, AkCas12b, AmCas12b, BsCas12b, OspCas12c, Cas12c2 (addgene plasmid #183072), Cas12c_4 (addgene plasmid #183071), Cas12c1 (addgene plasmid #120872), CasY.1 (from Katanobacteria), CasY.2 (from Vogelbacteria), CasY.3 (from Vogelbacteria), CasY.4 (from Parcubacteria), CasY.5 (from Komeilibacteria), CasY.6 (from Kerfeldbacteria), PlmCasX, DpbCasX, Un1Cas12f, CnCas12f1, enRhCas12f1, AsCas12f1, SpaCas12f1, Cas12g1 (addgene plasmid #120879), Cas12 h from the international application WO2021113522A1 (SEQ ID NO: 1 from the application), Cas12i1 (addgene plasmid #171670), Cas1212 (addgene plasmid #188275), Cas12i1 (addgene plasmid #120882), Cas12i2 (addgene plasmid #120883), Cas12i protein named as Cas12f.4/Cas12f.5/Cas12f.6 in CN111757889B, dSiCas12i (D1049A), SiCas12i, Si2Cas12i, WiCas12i, Wi2Cas12i, Wi3Cas12i, SaCas12i, Sa2Cas12i, Sa3Cas12i, WaCas12i, Wa2Cas12i, xCas12i, hfCas12Max, Cas12i-Max (addgene plasmid #188276), Cas12i1 D647A (addgene plasmid #171671), Cas12i-HiFi (addgene plasmid #188269), Cas12i1 D647A, Cas12j3 (addgene plasmid #188497), Cas12j2 (addgene plasmid #188498), AsCas12j-2 (addgene plasmid #191655), Cas12j-8 (addgene plasmid #194966), ShCas12k, N7Cas12k, AcCas12k, Cas12k-TniQ (addgene plasmid #181787), Cas12k-TnsC (addgene plasmid #181789), Cas121, MmCas12m, MmCas12m ΔZF (H549A, C552A), dCas12m-ΔZF (D485A, H549A, C552A), AcCas12n, dAcCas12n (D240), TnpB Actinomadura_cellulosilytica_strain_DSM_45823, TnpB Actinomadura_namibiensis_strain_DSM_44197, TnpB Actinomadura_umbrina_strain_DSM_43927_$, TnpB Actinoplanes_lobatus_strain_DSM_43150 (TnpB-1 and TnpB-2), TnpB Alicyclobacillus_macrosporagiidus_strain_DSM_17980, TnpB Haloactinospora_alba_Strain_DSM_45015, TnpB Lipingzhangella_halophila_strain_DSM_102030, TnpB Meiothermus_silvanus_DSM_9946, TnpB QNFX01000004, ISDra2 TnpB (PDB: 8H1J), KraIscB-1, AwaIscB, OgeuIscB, GtFz1 (from Gaillardia theta), SpuFz1 (from Spizellomyces punctatus), NlovFz2 (from Percolozoa Naegleria lovaniensis), and MmeFz2 (from Mercenaria mercenaria), fragments thereof, and mutations thereof or fragments of the mutations. In some embodiments, the DNA-binding polypeptide is able to cleave one strand of a double-stranded nucleic acid molecule (e.g., a double-stranded DNA molecule). Alternatively, the DNA-binding polypeptide is an RNA-guided nuclease with nickase activity. In some embodiments, the Cas enzyme cleaves a target strand of a double-stranded nucleic acid molecule, which means that the Cas enzyme cleaves the strand that is base-paired (complementary) with gRNA (e.g., sgRNA) bound to the Cas enzyme. In some embodiments, the nucleic acid-binding polypeptide is an RNA-guided nuclease with inactivated nuclease activity. In some embodiments, the nucleic acid-binding polypeptide is a completely inactivated mutant relative to nuclease activity of the wild-type RNA-guided nuclease, such as a dCas enzyme, which comprises but is not limited to Cas9, Cas12, Cas13, TnpB, IscB, IsrB, and Fancor with completely inactivated nuclease activity (which are referred to as dead Cas9, dead Cas12, dead Cas13, dead TnpB, dead IscB, dead IsrB, and dead Fancor); or fragments or mutations thereof, such as a SpRYs Cas9 mutant. In some embodiments of the present disclosure, the nucleic acid-binding polypeptide is a partially inactivated mutant relative to nuclease activity of a wild-type RNA-guided nuclease, such as an nCas enzyme, e.g., a polypeptide fragment that retains the nuclease activity for cleavage of a single strand of double-stranded DNA, which comprises but is not limited to nickase Cas9 and nickase Cas12.

Some embodiments of the present disclosure provide a guide polynucleotide comprising (i) a DR sequence having at least 50% sequence identity to the sequence shown in any one of SEQ ID NO: 54-583 or 704, and (ii) a guide sequence engineered to hybridize to a target nucleic acid. The DR sequence is linked to the guide sequence, and the guide polynucleotide forms a complex with a Cas12 protein, and guide sequence-specific binding of the complex to the target nucleic acid.

In some embodiments of the present disclosure, the DR sequence has at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequence shown in any one of SEQ ID NO: 54-583 or 704.

In some embodiments of the present disclosure, the DR sequence has at least 60% sequence identity to the sequence shown in any one of SEQ ID NO: 54-583 or 704.

In some embodiments of the present disclosure, the DR sequence has at least 65% sequence identity to the sequence shown in any one of SEQ ID NO: 54-583 or 704.

In some embodiments of the present disclosure, the DR sequence has at least 70% sequence identity to the sequence shown in any one of SEQ ID NO: 54-583 or 704.

In some embodiments of the present disclosure, the DR sequence has at least 75% sequence identity to the sequence shown in any one of SEQ ID NO: 54-583 or 704.

In some embodiments of the present disclosure, the DR sequence has at least 80% sequence identity to the sequence shown in any one of SEQ ID NO: 54-583 or 704.

In some embodiments of the present disclosure, the DR sequence has at least 85% sequence identity to the sequence shown in any one of SEQ ID NO: 54-583 or 704.

In some embodiments of the present disclosure, the DR sequence has at least 90% sequence identity to the sequence shown in any one of SEQ ID NO: 54-583 or 704.

In some embodiments of the present disclosure, the DR sequence has at least 95% sequence identity to the sequence shown in any one of SEQ ID NO: 54-583 or 704.

In some embodiments of the present disclosure, the DR sequence has at least 96% sequence identity to the sequence shown in any one of SEQ ID NO: 54-583 or 704.

In some embodiments of the present disclosure, the DR sequence has at least 97% sequence identity to the sequence shown in any one of SEQ ID NO: 54-583 or 704.

In some embodiments of the present disclosure, the DR sequence has at least 98% sequence identity to the sequence shown in any one of SEQ ID NO: 54-583 or 704.

In some embodiments of the present disclosure, the DR sequence has at least 100% sequence identity to the sequence shown in any one of SEQ ID NO: 54-583 or 704.

In some embodiments, the Cas12 protein is a Cas12 protein as described herein.

In some embodiments, the guide sequence comprises 15-60 nucleotides. In some embodiments, the guide sequence comprises 15-50 nucleotides. In some embodiments, the guide sequence comprises 15-40 nucleotides. In some embodiments, the guide sequence comprises 15-35 nucleotides. In some embodiments, the guide sequence comprises 15-30 nucleotides. In some embodiments, the guide sequence comprises 15-25 nucleotides. In some embodiments, the guide sequence comprises 18-25 nucleotides. In some embodiments, the guide sequence comprises 20-25 nucleotides. In some embodiments, the guide sequence comprises 18-22 nucleotides. In some embodiments, the guide sequence comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In some embodiments, the guide sequence hybridizes to the target nucleic acid, and the guide sequence is 90%-100% complementary to the target nucleic acid.

In some embodiments, the guide sequence hybridizes to the target nucleic acid.

In some embodiments, the guide sequence hybridizes to the target nucleic acid, and the guide sequence is mismatched to the target nucleic acid by no more than one nucleotide.

In some embodiments, the DR sequence comprises 15-100 nucleotides. In some embodiments, the DR sequence comprises 15-90 nucleotides. In some embodiments, the DR sequence comprises 15-80 nucleotides. In some embodiments, the DR sequence comprises 15-70 nucleotides. In some embodiments, the DR sequence comprises 15-60 nucleotides. In some embodiments, the guide sequence comprises 15-50 nucleotides. In some embodiments, the guide sequence comprises 15-40 nucleotides. In some embodiments, the guide sequence comprises 20-40 nucleotides. In some embodiments, the guide sequence comprises 20-30 nucleotides. In some embodiments, the guide sequence comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In some embodiments, the guide sequence is located at the 3′ end of the DR sequence.

In some embodiments, the guide sequence is located at the 5′ end of the DR sequence.

In some embodiments, the guide polynucleotide further comprises tracrRNA.

In some embodiments of the present disclosure, the tracrRNA sequence has at least 50% sequence identity to any one of the sequences shown in SEQ ID NO: 584-695. In some embodiments of the present disclosure, the tracrRNA sequence has at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of the sequences shown in SEQ ID NO: 584-695.

In some embodiments of the present disclosure, the tracrRNA sequence is selected from the sequences shown in SEQ ID NO: 584-695.

In some embodiments, the tracrRNA is complementarily paired with the DR sequence. In general, the complementary pairing is a complementary pairing for partial bases. In some embodiments, the tracrRNA interacts with the DR sequence.

In some embodiments, the tracrRNA sequence is linked to the DR sequence. In some embodiments, the tracrRNA sequence is linked to the DR sequence by a nucleotide sequence. In some embodiments, the tracrRNA sequence is linked to the DR sequence by a nucleotide sequence including 1-10 nucleotides. In some embodiments, the tracrRNA sequence is linked to the DR sequence by a nucleotide sequence including 1, 2, 3, 4, 5, 6, 7, 8, 9, or 10 nucleotides. In some embodiments, the tracrRNA sequence is linked to the DR sequence by a nucleotide sequence including 4 nucleotides. In some embodiments, the tracrRNA sequence is linked to the DR sequence by a 5′-GAAA-3′ linker.

In some embodiments, the tracrRNA sequence is located at the 3′ end of the DR sequence.

In some embodiments, the tracrRNA sequence is located at the 5′ end of the DR sequence.

In some embodiments, the tracrRNA comprises 10-200 nucleotides. In some embodiments, the tracrRNA comprises 10-190, 10-180, 10-170, 10-160, 10-150, 10-140, 10-130, 10-120, 10-110, 10-100, 10-90, 10-80, 10-70, 10-60, 10-50, 10-40, 10-30, 10-20, 20-100, 30-100, 40-100, 20-90, 20-80, 20-70, 20-60, 20-50, or 30-50 nucleotides. In some embodiments, the tracrRNA comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

SEQ ID NO: 1-53 shows the amino acid sequence of the Cas protein.

SEQ ID NO: 54-583 shows the DR sequence corresponding to the Cas protein. When more than one DR sequences corresponding to a particular Cas protein are listed, any one DR sequence may be used.

SEQ ID NO: 584-695 shows the tracrRNA sequence corresponding to the Cas protein. When SEQ ID NO: 584-695 does not list the tracrRNA sequence corresponding to a specific Cas protein, then gRNA may comprise only the guide sequence and the DR sequence, and do not comprise the tracrRNA sequence. When SEQ ID NO: 584-695 lists the tracrRNA sequence corresponding to the particular Cas protein, then the gRNA may comprise the guide sequence and the DR sequence and optionally comprise the tracrRNA sequence. When more than one tracrRNA sequences corresponding to the particular Cas protein are listed, any one of the tracrRNA sequences may be selected for use.

Some embodiments of the present disclosure provide an inactivated Cas12 mutant. The inactivated Cas12 mutant is a nuclease-inactivated mutant of the Cas12 protein as described in the present disclosure.

In the present disclosure, depending on the context, a reference scope of the Cas12 protein may encompass the inactivated Cas12 mutant. However, given the importance of the inactivated Cas12 mutant (non-limiting examples including that the inactivated Cas12 mutant is fused with a deaminase for single-base editing, fused with a transcriptional activation domain or a transcriptional repression domain for transcription regulation, etc.), the inactivated Cas12 mutant may be described separately and in detail herein; which does not imply that the reference scope of the Cas12 protein necessarily excludes the inactivated Cas12 mutant.

In some embodiments, the inactivated Cas12 mutant is a mutant in which the nuclease activity is completely inactivated, i.e., a dead Cas12 mutant (dCas12). The dCas12 only binds the target nucleic acid under the mediation of the guide polynucleotide and has no or negligible cleavage activity against the target nucleic acid. For example, a target nucleic acid cleavage efficiency of the dCas12 is no more than 20%, 15%, 10%, 5%, 4%, 3%, 2%, or 1% of the target nucleic acid cleavage efficiency of the Cas12 protein before inactivating mutation.

In some embodiments, the inactivated Cas12 mutant is a mutant in which the nuclease activity is partially inactivated. Further, the mutant with partially inactivated nuclease activity is a nickase Cas12 (nCas12), which binds the target nucleic acid under mediation of the guide polynucleotide, and then cleaves one single strand of the double-stranded target nucleic acid without cleaving the other single strand.

In some embodiments, the inactivated Cas12 mutant is a Cas12 protein with an inactivated Ruvc domain.

In some embodiments, the inactivated Cas12 mutant is a Cas12 protein with an inactivated Ruvc-I, Ruvc-II, or Ruvc-III domain.

In some embodiments, the inactivated Cas12 mutant is obtained by introducing the inactivating mutation into the Ruvc-I, Ruvc-II, or Ruvc-III domain of the Cas12 protein.

In some embodiments, the inactivating mutation is selected from one or more of D651A, E891A, and D1082A corresponding to the amino acid sequence as shown in SEQ ID NO: 696.

In some embodiments, the inactivating mutation is D651A, E891A, and D1082A corresponding to the amino acid sequence shown in SEQ ID NO: 696.

In some embodiments, a PAM sequence recognized by the inactivated Cas12 mutant is the same as the PAM sequence recognized by the Cas12 protein.

In some embodiments, the PAM sequence (5′→3′) recognized by the inactivated Cas12 mutant is selected from any one or more of the following: A, C, T, G, TA, TC, GN, AA, AG, TG, AN, GG, CG, TN, NT, NG, GT, NA, CC, AC, GC, AT, CT, GA, TT, CN, NC, CA, NTN, ANN, TTN, ATC, NAC, AGA, TGC, TCT, NGN, CGC, NTC, GCA, TCG, TTT, CCG, GGG, NAG, ACA, CGG, CNG, ACN, GTG, CNT, TTG, TCN, GGT, TNC, CCN, CGT, TGG, CGA, NGG, TCC, AGT, NCA, CAN, TCA, NNG, TAC, CCT, NTG, CGN, TGN, CAT, NGC, GNG, GNC, NNA, GAA, TTC, CTT, ATA, TAT, GCT, NCC, TTA, AGN, GNN, CAA, CAC, AGG, NTT, ANG, GNA, GTT, NGA, TAA, GTA, GGN, GNT, NCG, ATT, CCA, CNN, AAA, AAC, ATN, GAG, CTG, ACG, NAA, TAN, NAT, CNA, GCN, GTC, NCN, CTN, CNC, ANT, NNC, CAG, NAN, ATG, NCT, CCC, AAN, TGT, TNA, ACC, GAT, ACT, AAT, GGA, GAN, ANC, GAC, NNT, CTA, TNN, GCG, GTN, TNT, AAG, TAG, NGT, NTA, ANA, CTC, GCC, TGA, GGC, AGC, TNG, NGAA, GANC, GCNC, NTNT, TGGG, AAGG, AAGN, NTNN, TCGT, CNTG, NTGG, CCGN, ATAT, TGCA, NGGT, TGNT, NNTG, NCCG, ACAT, GNTG, CGCG, GACN, NTCG, TCNG, CTGC, TNNC, GGTN, CGNN, TCCA, AGCN, TNAG, GGAC, GATC, AANA, NATG, CCAG, NAAT, TCNT, CACT, CGGC, CGAN, CNCA, ATNT, NNNG, NGCT, CTGG, GGAN, NTNC, ATTC, AATG, CNTC, TGGN, NATC, GTCG, ACNC, GCNN, GACT, CTNT, NCTT, NAGG, NANC, CTTA, GTCT, ANAG, NGCN, CNNA, TCAG, ACAC, NCGG, TNNT, CAAG, ACCT, CCCA, GTNC, ANTC, GACC, AACG, TTAA, TCCG, CGCC, NCCN, TTNA, NCNT, NGCA, AGNN, AATC, GGGA, GNAN, NAGA, CGNA, GTAT, GTNA, ATNC, ACNA, GGAA, NTCC, GGCG, AATN, CNNT, AGGC, GCGN, GTGC, TTGA, AAGC, GAAG, ATNG, TGCT, TACT, CTAN, GGCT, GNGC, GTCN, CGAA, CNAC, GCCT, TAGG, ANGC, TNAA, GANT, NCNA, NCCT, AGAN, GTAA, TTTN, ATGA, TGNA, CANC, ACGA, CCAC, CCGG, CTNG, CNGN, GGTA, NGNC, GTTT, CTAA, TNCT, CTGN, NGAC, TGTA, TANN, GCNT, GCTC, CNCG, AAAN, CCNT, GANA, CACA, CTNA, ANTN, TTNT, CCTG, TNTT, CANA, NTAN, CACG, GGAT, TTTC, GNCG, TACA, GTAC, GAGC, ACNN, ATGG, AANT, ATCC, ACCG, AGNC, TGTT, NCAT, ATTA, GNTT, GAGN, TNAC, GCCG, NTNG, GTGG, GNGN, ACCA, NTAA, ACTN, NCTG, NCTA, TTTT, GCNG, NTAG, CAAA, GGNA, CNTN, TTAG, TCTG, NCTN, TATG, GCGT, TANT, GGGT, NACN, ACTG, CCNG, GNNT, CCAT, GNTA, NANT, TACN, TGTN, ATCT, NCAN, TNGG, CNNN, AAGT, ATTN, GGNN, CAGC, CGTN, GCCC, GCTT, CNAT, NANA, CCNN, GNGA, TNGN, GCAG, CGNG, CCTT, NGAG, NCNG, AANG, GGTC, ACTC, TGAA, NAGN, NNCA, ACGG, TGAC, TCCN, ANNN, TCGN, TAAN, CAGG, TTAN, NGAN, NTGC, CCNC, TNTN, ATGN, GTGN, GCAT, NNGN, NNCC, CCNA, CNAG, GNAC, CGNT, TTCN, TAGN, ANCT, NATN, GTGA, TNGT, CTAT, CCCG, TNCA, NGTA, NNGA, CGTG, TAAT, CGCA, NNCG, NGTC, NAGT, GNAT, TNTC, NCGC, NGGN, CATN, GTTN, AGTA, GNNG, TTNN, TGNC, NAAA, TNCC, CACC, CTCT, TTGN, GCTA, NTTT, TGAN, TNAN, NGAT, CCTN, GAAT, GTCA, NTCN, GCCA, ANTG, TGGC, CAAC, TTTA, TGTC, CGGA, NCGN, AGNT, NCGA, ANCG, ACAA, TAGT, CGAG, NCAA, AATA, AGGG, GNGT, CAGA, AGGT, GGGG, ANAC, TGGT, GTGT, GNCA, GTTA, NGTT, TNNG, NCAG, CACN, GCAN, GAAC, NCCA, TTCC, NCNN, GNNN, ANGT, NTNA, CCCT, GNAA, TTNG, GTNN, GGNG, TCTA, NCAC, GANG, TTCG, CCTC, CNGG, ANNA, TCAN, ATCG, NTGA, CGTA, TTAC, GCTN, GCTG, NGTG, TCCC, CANN, NNNA, TAGA, ACGT, AGAT, GATG, GCCN, TGNG, GCGC, CCGA, GNCN, NTTG, NNAT, TNCG, NANG, GGTG, NCCC, GNCC, CAAT, CGCN, CNGA, NTTC, TTCT, NGGA, AGTC, CNNC, NACG, AGTN, NANN, ACAG, GNCT, TACC, CNTA, TGTG, CATC, GACA, TCTT, NTCT, CTGA, AGGA, GATA, TNAT, CCTA, GGAG, ANCC, AANC, GTAN, GCNA, TGNN, TANC, GNTN, AGCG, CTAG, NNAA, AGTT, CTAC, TACG, TTNC, TNTA, ANTT, ATAC, TCCT, TCAC, NGGC, NTTN, NNTC, CANT, ATAA, TGCC, CTCC, TNNA, GING, ACGN, GGCA, AAAG, TTGT, NGNA, NAAN, TATN, CGGG, CATA, ATGC, ACGC, ACCN, ATTT, TCNA, TNGC, NACA, NACC, CTCN, GGCC, TANG, AGAA, TNGA, TAGC, CAGN, GGCN, ANNT, NNNC, TCAT, CATT, TAAA, ATGT, TGAG, CGCT, TCGG, GCAC, GTAG, NTCA, NATT, ANTA, CCCN, ACTA, AAAA, GAAN, TATT, NNAC, TGAT, GGGN, CCAA, GNGG, CCAN, GTCC, NNCT, AGNG, CNTT, CNCT, GANN, GGTT, AGCT, CATG, NTAC, TNCN, NNTN, TGGA, GATT, AGCA, TAAG, GCGA, ACTT, ANGN, NTGN, AACN, AACT, TCAA, NTAT, TCGA, NCTC, NNGG, ANGG, NNTT, GTNT, CTNN, CGGN, TAAC, GGNC, GAAA, ACNG, GNAG, TTGG, CTTC, CNGT, TNNN, TNTG, GTTG, TCNN, CGGT, GAGA, CNNG, NCNC, GAGG, AGCC, ATNN, NNNT, AGAC, AACC, ANNC, ANNG, ACAN, GTTC, TATA, GNTC, NCGT, NGNT, CGTC, CCGC, CGAC, GACG, ATTG, GNNC, CNAA, TATC, AGNA, CTNC, TTCA, ANCA, ACCC, AGTG, CCGT, ANAT, CTGT, GGGC, NTTA, NAAG, AANN, CNAN, NNCN, ANAA, ANAN, CTTG, NGNN, AGAG, TANA, TCNC, GCAA, NGNG, NAGC, NATA, ATCN, CGTT, CNGC, GATN, NNTA, AAGA, CTTT, AAAC, AGGN, ACNT, NTGT, CTTN, ATCA, NACT, NNAG, NGTN, NAAC, TGCG, GGNT, ATAN, TTGC, ANCN, CCCC, ANGA, NGCG, TCTC, CTCG, ATNA, AATT, NNAN, NNGT, TCGC, ATAG, CAAN, AACA, TTAT, CAGT, GNNA, TGCN, GCGG, NGGG, CANG, TTTG, GAGT, AAAT, CTCA, CNCN, CNCC, TCTN, CGNC, NGCC, CGAT, and NNGC.

N is A, T, C, or G.

Some embodiments of the present disclosure provide a fusion protein or conjugate. The fusion protein or conjugate comprises: (1) the Cas12 protein as described herein, or the inactivated Cas12 mutant as described herein; and (2) a homologous or heterologous functional domain.

In the present disclosure, depending on the context, the reference scope of the Cas12 protein may encompass the inactivated Cas12 mutant. However, given the importance of the inactivated Cas12 mutant (non-limiting examples including the fusion of the inactivated Cas12 mutant with the deaminase for single base editing, fusion with a transcriptional activation domain or a transcriptional repression domain for transcriptional regulation, etc.), the inactivated Cas12 mutant is described separately and in detail herein, which does not imply that the reference scope of the Cas12 protein necessarily excludes the inactivated Cas12 mutant.

In some embodiments of the present disclosure, a fusion protein is provided. The fusion protein comprises (1) the Cas12 protein as described herein, or the inactivated Cas12 mutant as described herein; and (2) the homologous or heterologous functional domain.

In some embodiments of the present disclosure, a fusion protein is provided. The fusion protein comprises (1) the Cas12 protein as described herein; and (2) the homologous or heterologous functional domain.

In some embodiments, a conjugate is provided. The conjugate comprises (1) the Cas12 protein as described herein, or the inactivated Cas12 mutant as described herein; and (2) the homologous or heterologous functional domain.

In some embodiments, a conjugate is provided. The conjugate comprises (1) the Cas12 protein as described herein; and (2) the homologous or heterologous functional domain.

In some embodiments, the functional domain has an enzyme activity for modifying the target nucleic acid sequence; the enzyme activity comprising a nuclease activity, a methyltransferase activity, a demethylase activity, a DNA repair activity, a DNA damage activity, a deamination activity, a dismutase activity, an alkylation activity, a depurination activity, an oxidation activity, a pyrimidine dimer formation activity, an integrase activity, a transposase activity, a recombinase activity, a polymerase activity, a ligase activity, a helicase activity, a photolyase activity, a glycosylase activity, a deglycosylation activity, an acetyltransferase activity, a deacetylase activity, a kinase activity, a phosphatase activity, a ubiquitin ligase activity, a deubiquitination activity, an adenylylation activity, a deadenylation activity, a SUMOylating activity, a deSUMOylating activity, a myristoylation activity, and/or a demyristoylation activity.

In some embodiments, the inactivating mutation is selected from one or more of D651A, E891A, and D1082A corresponding to the amino acid sequence shown in SEQ ID NO: 696.

In some embodiments, the inactivating mutation is D651A, E891A, and D1082A corresponding to the amino acid sequence as shown in SEQ ID NO: 696.

In some embodiments, the functional domain is selected from one or more of the following: a nuclease (e.g., FokI), a DNA methyltransferase, a DNA demethylase, a histone methyltransferase, a histone demethylase, a histone acetylase domain, a histone deacetylase domain, a DNA repair enzyme, a DNA damage enzyme, a deaminase, a dismutase, an alkylase, a depurinase, an oxidase, a pyrimidine dimer-forming enzyme, an integrase, a transposase, a recombinase, a polymerase, a ligase, a helicase, a photolyase, a glycosylase, a deglycosylase, an acetyltransferase, a deacetylase, a kinase, a phosphatase, a ubiquitin ligase, a deubiquitinating enzyme, an adenylylase, a deadenylase, a SUMOylating enzyme, a deSUMOylating enzyme, a myristoylase, and/or a demyristoylase.

In some embodiments, the homologous or heterologous functional domain is selected from any one, two, three, four, or more of the following: a subcellular positioning signal, a DNA binding domain, a protease domain, a transcriptional activation domain, a transcriptional repression domain, a nuclease domain, a deaminase domain, a uracil DNA glycosylase domain (UDG), a uracil DNA glycosylase inhibitory domain (UGI), a DNA methyltransferase, a DNA demethylase, a histone methyltransferase, a histone demethylase, a transcription release factor, a histone acetylase domain, a histone deacetylase domain, a DNA ligase, an affinity tag, a reporter tag, an affinity domain, and a reporter domain.

In some embodiments, the homologous or heterologous functional domain comprises a transcriptional repressor. In some embodiments, the homologous or heterologous functional domain comprises a DNA methyltransferase. In some embodiments, the homologous or heterologous functional domain comprises a histone methyltransferase. In some embodiments, the homologous or heterologous functional domain comprises a histone domain. In some embodiments, the homologous or heterologous functional domain is selected from one, two, three, or more of the following: a transcriptional repressor, a DNA methyltransferase, a histone methyltransferase, and a histone domain.

In some embodiments, the transcriptional repressor is randomly selected from: Kruppel-associated Box (KRAB), Enhancer of Zeste Homolog 2 (EZH2), Zinc Finger Protein 57 (ZFP57), Zinc Finger Protein 445 (ZNF445), Tripartite Motif Containing 28 (TRIM28, also known as KAP1), Methyl-CpG Binding Protein (MeCP, such as MeCP2), Sin3 Interaction Domain (SID), Tandem Repeat of Sin3 Interaction Domain (SID4X), Methyl-CpG Binding Domain Protein 2 (MBD2), Methyl-CpG Binding Domain Protein 3 (MBD3), DNA Methyltransferase 1 (DNMT1), DNA Methyltransferase 3 Alpha (DNMT3A), DNA Methyltransferase 3 Beta (DNMT3B), RE1-Silencing Transcription Factor (REST), Neuron-Restrictive Silencer Factor (NRSF, alias for REST), TGF-β-Inducible Early Gene (TIEG, also known as KLF10), Corepressor of REST (CoREST), G9a (Euchromatic Histone-Lysine N-Methyltransferase 2, also known as EHMT2), Suppressor of Variegation 3-9 Homolog 1 (SUV39H1), SET Domain, Bifurcated 1 (SETDB1), Histone Deacetylase 1 (HDAC1), Histone Deacetylase 2 (HDAC2), Histone Deacetylase 3 (HDAC3), Silencing Mediator for Retinoid and Thyroid Hormone Receptors (SMRT), Nuclear Receptor Corepressor (NCoR), ERG-Associated Protein with SET Domain (ESET, also referred to as SETDB1), Zinc Finger and BTB Domain Containing 33 (ZBTB33, also known as KAISO), Viral Oncogene Homolog of Thyroid Hormone Receptor (v-ERB-A), Transducin Beta-Like 1 (TBL1), Transducin Beta-Like Related 1 (TBLR1), Lysine-Specific Histone Demethylase 1A (LSD1, also known as KDM1A), C-terminal Binding Protein 1 (CtBP1), C-terminal Binding Protein 2 (CtBP2), Arabidopsis Histone Deacetylase 2A (AtHD2A), BCL6 Corepressor (BCOR), Mesoderm Induction Early Response 1 (MIER1), Zinc Finger Protein 10 (ZNF10), Zinc Finger Protein 91 (ZNF91), Zinc Finger Protein 809 (ZFP809), Bromo Adjacent Homology Domain Containing 1 (BAHD1), Retinoblastoma Binding Protein 4 (RBBP4), Retinoblastoma Binding Protein 7 (RBBP7), Heterochromatin Protein 1 Alpha (HP1α, also known as CBX5), Heterochromatin Protein 1 Beta (HP1β, also known as CBX1), Chromobox Protein Homolog 3 (CBX3), Chromobox Protein Homolog 7 (CBX7), PR Domain Zinc Finger Protein 1 (PRDM1, also known as BLIMP-1), PR Domain Zinc Finger Protein 14 (PRDM14), Nuclear Receptor Corepressor 2 (NCOR2, also known as SMRT), Metastasis Associated 1 (MTA1), Metastasis Associated 2 (MTA2), Metastasis Associated 3 (MTA3), Chromodomain Helicase DNA Binding Protein 4 (CHD4), and Lysine Demethylase 5B, also known as JARID1B (KDM5B).

In some embodiments, the DNA methyltransferase is selected from DNMT3A, DNMT3B, DNMT3L, DRM, UHRF1, DNMT1, dim-2, M.Sssl, M.Pvull (N4C), DMS3, and T4Dam (N6C).

In some embodiments, the DNA methyltransferase is selected from DNA (Cytosine-5)-Methyltransferase 1 (DNMT1), DNA (Cytosine-5)-Methyltransferase 3 Alpha (DNMT3A), DNA (Cytosine-5)-Methyltransferase 3 Beta (DNMT3B), DNA (Cytosine-5)-Methyltransferase 3-Like (DNMT3L), tRNA Aspartic Acid Methyltransferase 1 (DNMT2), Ubiquitin-like with PHD and RING Finger Domains 1 (UHRF1), Ubiquitin-like with PHD and RING Finger Domains 2 (UHRF2), Helicase, Lymphoid Specific (HELLS, also known as LSH), Cell Division Cycle Associated 7 (CDCA7), Nuclear Protein 95 (NP95, the obsolete name of UHRF1), tRNA aspartic acid methyltransferase 1 (TRDMT1, alias for DNMT2), DNA (Cytosine-5)-Methyltransferase 3C (DNMT3C, a new type of DNA methyltransferase unique to mice, used for transposon silencing in male germ cells), and KIAA1429 (also known as VIRMA, which regulates m6A but may be related to methylation regulation). In some embodiments, the DNA methyltransferase is selected from Methyltransferase 1 (MET1, functionally analogous to mammalian DNMT1), Chromomethylase 3 (CMT3, mediating CHG site methylation), Chromomethylase 2 (CMT2, mediating CHH methylation), Domains Rearranged Methyltransferase 1 (DRM1), and Domains Rearranged Methyltransferase 2 (DRM2, functionally analogous to mammalian DNMT3, mediating de novo methylation). In some embodiments, the DNA methyltransferase is selected from Repeat-Induced Point mutation defective protein (RID, involved in RIP mutation and DNA methylation) and Methyltransferase associated with sexual cycle 1 (Masc1). In some embodiments, the DNA methyltransferase is selected from CpG-specific DNA methyltransferase from Spiroplasma species (M.SssI, intended to be used as a model enzyme for specifically methylation of CpG sites), DNA adenine methyltransferase (Dam, an adenine methyltransferase for methylation of GATC sites, commonly used in prokaryotic regulation research), and DNA cytosine methyltransferase (Dcm, a cytosine methyltransferase for specifically methylation of the CCWGG sequence).

In some embodiments, the histone domain is selected from a histone H3 domain, a histone H2 domain, and a histone H4 domain. In some embodiments, the histone domain is selected from a histone H3 tail domain, a histone H2 tail domain, and a histone H4 tail domain.

In some embodiments of the present disclosure, the subcellular positioning signal is selected from a nuclear localization signal, a nuclear export signal, a mitochondrial localization signal, and a chloroplast localization signal.

In some embodiments, the fusion protein or conjugate comprises 1, 2, 3, 4, 5, 6, 7, 8, 9, or more homologous or heterologous functional domains, and the functional domains are identical or different.

In some embodiments, the fusion protein or conjugate connects 0, 1, 2, 3, 4, 5, 6, 7, 8, or more functional domains at the N-terminal and/or C-terminal of the Cas12 protein.

In some embodiments, the fusion protein comprises 1, 2, 3, 4, or more nuclear positioning signals.

In some embodiments, the fusion protein is used to achieve base editing, such as in conjunction with the guide polynucleotide to achieve the base editing. In some embodiments, the fusion protein comprises the nuclear positioning signal and the deaminase domain.

In some embodiments, the fusion protein comprises the nuclear positioning signal, a cytidine deaminase domain, and optionally 1 or 2 UGI domains. The fusion protein is used to achieve C→T base editing of the target nucleic acid.

In some embodiments, the fusion protein comprises the nuclear positioning signal and the adenosine deaminase domain. The fusion protein is used to achieve A→G base editing of the target nucleic acid.

In some embodiments, the fusion protein comprises the nuclear positioning signal, the cytidine deaminase domain, and the adenosine deaminase domain. In some embodiments, the fusion protein comprises 1, 2, or 3 nuclear positioning signals, and the deaminase domain. In some embodiments, the fusion protein comprises the UGI domain. In some embodiments, the fusion protein comprises 1, 2, or 3 nuclear positioning signals, the deaminase domain, and 1 or 2 UGI domains.

In some embodiments, the fusion protein is used to achieve the transcriptional activation of a specific target gene, such as in conjunction with the guide polynucleotide for achieving the transcriptional activation of the specific target gene. In some embodiments, the fusion protein comprises the nuclear positioning signal and the transcriptional activation domain.

In some embodiments, the fusion protein is used to achieve the transcriptional repression of a specific target gene, such as in conjunction with the guide polynucleotide for achieving the transcriptional repression of the specific target gene. In some embodiments, the fusion protein comprises the nuclear positioning signal and the transcriptional repression domain.

In some embodiments, the fusion protein is used to achieve methylation of a specific target sequence, such as in conjunction with the guide polynucleotide for achieving methylation of the specific target sequence. In some embodiments, the fusion protein comprises the nuclear positioning signal and the DNA methylation domain.

In some embodiments, the fusion protein is used to achieve demethylation of a specific target sequence, such as in conjunction with the guide polynucleotide for achieving demethylation of the specific target sequence. In some embodiments, the fusion protein comprises the nuclear positioning signal and the DNA demethylation domain.

In some embodiments, the nuclease domain comprises a polypeptide with an ssDNA cleavage activity and/or a polypeptide with a dsDNA cleavage activity.

In some embodiments, the nuclease domain comprises a polypeptide with an ssDNA cleavage activity.

In some embodiments, the nuclease domain comprises a polypeptide with a dsDNA cleavage activity.

In some embodiments, the Cas12 protein or inactivated Cas12 mutant is directly or indirectly linked to the homologous or heterologous functional domain.

In some embodiments, the direct linkage is a covalent linkage, and the indirect linkage is a linkage through an amino acid linker or a non-amino acid linker.

In some embodiments, the homologous or heterologous functional domain is fused or conjugated at the N-terminal, C-terminal, or internally with respect to the Cas12 protein or inactivated Cas12 mutant.

In the present disclosure, the fusion protein is obtained by connecting (1) to (2) through a peptide linker or directly connecting (1) to (2); and the conjugate is obtained by connecting (1) to (2) through a non-peptide chemical bond.

In some embodiments, the PAM sequence recognized by the fusion protein or the conjugate is the same as the PAM sequence recognized by the Cas12 protein.

In some embodiments, the PAM sequence (5′→3′) recognized by the fusion protein or conjugate is selected from any one or more of the following: A, C, T, G, TA, TC, GN, AA, AG, TG, AN, GG, CG, TN, NT, NG, GT, NA, CC, AC, GC, AT, CT, GA, TT, CN, NC, CA, NTN, ANN, TTN, ATC, NAC, AGA, TGC, TCT, NGN, CGC, NTC, GCA, TCG, TTT, CCG, GGG, NAG, ACA, CGG, CNG, ACN, GTG, CNT, TTG, TCN, GGT, TNC, CCN, CGT, TGG, CGA, NGG, TCC, AGT, NCA, CAN, TCA, NNG, TAC, CCT, NTG, CGN, TGN, CAT, NGC, GNG, GNC, NNA, GAA, TTC, CTT, ATA, TAT, GCT, NCC, TTA, AGN, GNN, CAA, CAC, AGG, NTT, ANG, GNA, GTT, NGA, TAA, GTA, GGN, GNT, NCG, ATT, CCA, CNN, AAA, AAC, ATN, GAG, CTG, ACG, NAA, TAN, NAT, CNA, GCN, GTC, NCN, CTN, CNC, ANT, NNC, CAG, NAN, ATG, NCT, CCC, AAN, TGT, TNA, ACC, GAT, ACT, AAT, GGA, GAN, ANC, GAC, NNT, CTA, TNN, GCG, GTN, TNT, AAG, TAG, NGT, NTA, ANA, CTC, GCC, TGA, GGC, AGC, TNG, NGAA, GANC, GCNC, NTNT, TGGG, AAGG, AAGN, NTNN, TCGT, CNTG, NTGG, CCGN, ATAT, TGCA, NGGT, TGNT, NNTG, NCCG, ACAT, GNTG, CGCG, GACN, NTCG, TCNG, CTGC, TNNC, GGTN, CGNN, TCCA, AGCN, TNAG, GGAC, GATC, AANA, NATG, CCAG, NAAT, TCNT, CACT, CGGC, CGAN, CNCA, ATNT, NNNG, NGCT, CTGG, GGAN, NTNC, ATTC, AATG, CNTC, TGGN, NATC, GTCG, ACNC, GCNN, GACT, CTNT, NCTT, NAGG, NANC, CTTA, GTCT, ANAG, NGCN, CNNA, TCAG, ACAC, NCGG, TNNT, CAAG, ACCT, CCCA, GTNC, ANTC, GACC, AACG, TTAA, TCCG, CGCC, NCCN, TTNA, NCNT, NGCA, AGNN, AATC, GGGA, GNAN, NAGA, CGNA, GTAT, GTNA, ATNC, ACNA, GGAA, NTCC, GGCG, AATN, CNNT, AGGC, GCGN, GTGC, TTGA, AAGC, GAAG, ATNG, TGCT, TACT, CTAN, GGCT, GNGC, GTCN, CGAA, CNAC, GCCT, TAGG, ANGC, TNAA, GANT, NCNA, NCCT, AGAN, GTAA, TTTN, ATGA, TGNA, CANC, ACGA, CCAC, CCGG, CTNG, CNGN, GGTA, NGNC, GTTT, CTAA, TNCT, CTGN, NGAC, TGTA, TANN, GCNT, GCTC, CNCG, AAAN, CCNT, GANA, CACA, CTNA, ANTN, TTNT, CCTG, TNTT, CANA, NTAN, CACG, GGAT, TTTC, GNCG, TACA, GTAC, GAGC, ACNN, ATGG, AANT, ATCC, ACCG, AGNC, TGTT, NCAT, ATTA, GNTT, GAGN, TNAC, GCCG, NING, GTGG, GNGN, ACCA, NTAA, ACTN, NCTG, NCTA, TTTT, GCNG, NTAG, CAAA, GGNA, CNTN, TTAG, TCTG, NCTN, TATG, GCGT, TANT, GGGT, NACN, ACTG, CCNG, GNNT, CCAT, GNTA, NANT, TACN, TGTN, ATCT, NCAN, TNGG, CNNN, AAGT, ATTN, GGNN, CAGC, CGTN, GCCC, GCTT, CNAT, NANA, CCNN, GNGA, TNGN, GCAG, CGNG, CCTT, NGAG, NCNG, AANG, GGTC, ACTC, TGAA, NAGN, NNCA, ACGG, TGAC, TCCN, ANNN, TCGN, TAAN, CAGG, TTAN, NGAN, NTGC, CCNC, TNTN, ATGN, GTGN, GCAT, NNGN, NNCC, CCNA, CNAG, GNAC, CGNT, TTCN, TAGN, ANCT, NATN, GTGA, TNGT, CTAT, CCCG, TNCA, NGTA, NNGA, CGTG, TAAT, CGCA, NNCG, NGTC, NAGT, GNAT, TNTC, NCGC, NGGN, CATN, GTTN, AGTA, GNNG, TTNN, TGNC, NAAA, TNCC, CACC, CTCT, TTGN, GCTA, NTTT, TGAN, TNAN, NGAT, CCTN, GAAT, GTCA, NTCN, GCCA, ANTG, TGGC, CAAC, TTTA, TGTC, CGGA, NCGN, AGNT, NCGA, ANCG, ACAA, TAGT, CGAG, NCAA, AATA, AGGG, GNGT, CAGA, AGGT, GGGG, ANAC, TGGT, GTGT, GNCA, GTTA, NGTT, TNNG, NCAG, CACN, GCAN, GAAC, NCCA, TTCC, NCNN, GNNN, ANGT, NTNA, CCCT, GNAA, TTNG, GTNN, GGNG, TCTA, NCAC, GANG, TTCG, CCTC, CNGG, ANNA, TCAN, ATCG, NTGA, CGTA, TTAC, GCTN, GCTG, NGTG, TCCC, CANN, NNNA, TAGA, ACGT, AGAT, GATG, GCCN, TGNG, GCGC, CCGA, GNCN, NTTG, NNAT, TNCG, NANG, GGTG, NCCC, GNCC, CAAT, CGCN, CNGA, NTTC, TTCT, NGGA, AGTC, CNNC, NACG, AGTN, NANN, ACAG, GNCT, TACC, CNTA, TGTG, CATC, GACA, TCTT, NTCT, CTGA, AGGA, GATA, TNAT, CCTA, GGAG, ANCC, AANC, GTAN, GCNA, TGNN, TANC, GNTN, AGCG, CTAG, NNAA, AGTT, CTAC, TACG, TTNC, TNTA, ANTT, ATAC, TCCT, TCAC, NGGC, NTTN, NNTC, CANT, ATAA, TGCC, CTCC, TNNA, GING, ACGN, GGCA, AAAG, TTGT, NGNA, NAAN, TATN, CGGG, CATA, ATGC, ACGC, ACCN, ATTT, TCNA, TNGC, NACA, NACC, CTCN, GGCC, TANG, AGAA, TNGA, TAGC, CAGN, GGCN, ANNT, NNNC, TCAT, CATT, TAAA, ATGT, TGAG, CGCT, TCGG, GCAC, GTAG, NTCA, NATT, ANTA, CCCN, ACTA, AAAA, GAAN, TATT, NNAC, TGAT, GGGN, CCAA, GNGG, CCAN, GTCC, NNCT, AGNG, CNTT, CNCT, GANN, GGTT, AGCT, CATG, NTAC, TNCN, NNTN, TGGA, GATT, AGCA, TAAG, GCGA, ACTT, ANGN, NTGN, AACN, AACT, TCAA, NTAT, TCGA, NCTC, NNGG, ANGG, NNTT, GTNT, CTNN, CGGN, TAAC, GGNC, GAAA, ACNG, GNAG, TTGG, CTTC, CNGT, TNNN, TNTG, GTTG, TCNN, CGGT, GAGA, CNNG, NCNC, GAGG, AGCC, ATNN, NNNT, AGAC, AACC, ANNC, ANNG, ACAN, GTTC, TATA, GNTC, NCGT, NGNT, CGTC, CCGC, CGAC, GACG, ATTG, GNNC, CNAA, TATC, AGNA, CTNC, TTCA, ANCA, ACCC, AGTG, CCGT, ANAT, CTGT, GGGC, NTTA, NAAG, AANN, CNAN, NNCN, ANAA, ANAN, CTTG, NGNN, AGAG, TANA, TCNC, GCAA, NGNG, NAGC, NATA, ATCN, CGTT, CNGC, GATN, NNTA, AAGA, CTTT, AAAC, AGGN, ACNT, NTGT, CTTN, ATCA, NACT, NNAG, NGTN, NAAC, TGCG, GGNT, ATAN, TTGC, ANCN, CCCC, ANGA, NGCG, TCTC, CTCG, ATNA, AATT, NNAN, NNGT, TCGC, ATAG, CAAN, AACA, TTAT, CAGT, GNNA, TGCN, GCGG, NGGG, CANG, TTTG, GAGT, AAAT, CTCA, CNCN, CNCC, TCTN, CGNC, NGCC, CGAT, and NNGC.

N is A, T, C, or G.

In some embodiments, a PAM sequence recognized by the fusion protein is 5′-TTN-3′.

In some embodiments, a PAM sequence recognized by the fusion protein is 5′-TTNC-3′.

In some embodiments, a PAM sequence recognized by the fusion protein is 5′-WTN-3′.

In some embodiments, a PAM sequence recognized by the fusion protein is 5′-ATN-3′.

In some embodiments, a PAM sequence recognized by the fusion protein is 5′-TTN-3′.

In some embodiments, a PAM sequence recognized by the fusion protein is 5′-TTNC-3′.

In some embodiments, a PAM sequence recognized by the fusion protein is 5′-WTN-3′.

In some embodiments, a PAM sequence recognized by the fusion protein is 5′-ATN-3′.

Some embodiments of the present disclosure provide an isolated nucleic acid. The nucleic acid encodes the Cas12 protein, the inactivated Cas12 mutant, or the fusion protein or conjugate as described herein.

In some embodiments of the present disclosure, the nucleic acid encodes the Cas12 protein or the fusion protein as described herein.

In some embodiments, the nucleic acid is codon optimized for expression in cells.

In some embodiments, the nucleic acid is codon optimized for expression in a eukaryote, a mammal such as a human or non-human mammal, a plant, an insect, a bird, a reptile, a rodent (e.g., a mouse, a rat), a fish, a worm/nematode, or a yeast.

Some embodiments of the present disclosure provide a CRISPR-Cas12 system. In some embodiments, the CRISPR-Cas12 system comprises:
a. the Cas12 protein, the inactivated Cas12 mutant, the fusion protein or conjugate, or the isolated nucleic acid as described herein; and
b. a guide polynucleotide, or a polynucleotide sequence encoding the guide polynucleotide.

The Cas12 protein, the inactivated Cas12 mutant, or the fusion protein or conjugate forms a complex with the guide polynucleotide; and the guide polynucleotide comprises a guide sequence engineered to guide a sequence-specific binding of the complex to the target nucleic acid.

The isolated nucleic acid encodes the Cas12 protein, the inactivated Cas12 mutant, or the fusion protein or conjugate as described herein.

In some embodiments, the guide polynucleotide comprises a DR sequence linked to a guide sequence.

In some embodiments, the DR sequence has at least 50% sequence identity to the sequence shown in any one of SEQ ID NO: 54-583 or 704.

In some embodiments, the guide polynucleotide comprises a DR sequence linked to a guide sequence. Further, in some embodiments, the DR sequence has at least 50% sequence identity to the sequence shown in any one of SEQ ID NO: 54-583 or 704. In some embodiments, the DR sequence has at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in any one of SEQ ID NO: 54-583 or 704. Further, in some embodiments, the DR sequence comprises or is the sequence shown in any one of SEQ ID NO: 54-583 or 704.

In some embodiments, the guide sequence comprises 15-60 nucleotides. In some embodiments, the guide sequence comprises 15-50 nucleotides. In some embodiments, the guide sequence comprises 15-40 nucleotides. In some embodiments, the guide sequence comprises 15-35 nucleotides. In some embodiments, the guide sequence comprises 15-30 nucleotides. In some embodiments, the guide sequence comprises 15-25 nucleotides. In some embodiments, the guide sequence comprises 18-25 nucleotides. In some embodiments, the guide sequence comprises 20-25 nucleotides. In some embodiments, the guide sequence comprises 18-22 nucleotides. In some embodiments, the guide sequence comprises 20-22 nucleotides. In some embodiments, the guide sequence comprises 15, 16, 17, 18, 19, 20, 21, 22, 23, 24, 25, 26, 27, 28, 29, 30, 31, 32, 33, 34, 35, 36, 37, 38, 39, or 40 nucleotides.

In some embodiments, the guide sequence hybridizes to the target nucleic acid, and the guide sequence is 90%-100% complementary to the target nucleic acid.

In some embodiments, the guide sequence hybridizes to the target nucleic acid.

In some embodiments, the guide sequence hybridizes to the target nucleic acid, and the guide sequence is mismatched to the target nucleic acid by no more than one nucleotide.

In some embodiments, the DR sequence comprises 15-100 nucleotides. In some embodiments, the DR sequence comprises 15-90 nucleotides. In some embodiments, the DR sequence comprises 15-80 nucleotides. In some embodiments, the DR sequence comprises 15-70 nucleotides. In some embodiments, the DR sequence comprises 15-60 nucleotides. In some embodiments, the guide sequence comprises 15-50 nucleotides. In some embodiments, the guide sequence comprises 15-40 nucleotides. In some embodiments, the guide sequence comprises 20-40 nucleotides. In some embodiments, the guide sequence comprises 20-30 nucleotides. In some embodiments, the guide sequence comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

In some embodiments, the guide sequence is located at the 3′ end of the DR sequence.

In some embodiments, the guide sequence is located at the 5′ end of the DR sequence.

In some embodiments, the guide polynucleotide further comprises the tracrRNA.

In some embodiments of the present disclosure, the tracrRNA sequence has at least 50% sequence identity to the sequence shown in any one of SEQ ID NO: 584-695. In some embodiments of the present disclosure, the tracrRNA sequence has at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to the sequence shown in any one of SEQ ID NO: 584-695.

In some embodiments, the tracrRNA is complementarily paired with the DR sequence. In general, the complementary pairing is complementary pairing for partial bases. In some embodiments, the tracrRNA interacts with the DR sequence.

In some embodiments, the tracrRNA sequence is linked to the DR sequence. In some embodiments, the tracrRNA sequence is linked to the DR sequence by a nucleotide sequence. In some embodiments, the tracrRNA sequence is linked to the DR sequence by the nucleotide sequence including 1-10 nucleotides. In some embodiments, the tracrRNA sequence is linked to the DR sequence by the nucleotide sequence including 1, 2, 3, 4, 5, 6, 7, 8, 9, or 10 nucleotides. In some embodiments, the tracrRNA sequence is linked to the DR sequence by the nucleotide sequence including 4 nucleotides. In some embodiments, the tracrRNA sequence is linked to the DR sequence by a 5′-GAAA-3′ sequence.

In some embodiments, the tracrRNA sequence is located at the 3′ end of the DR sequence.

In some embodiments, the tracrRNA sequence is located at the 5′ end of the DR sequence.

In some embodiments, the tracrRNA comprises 10-200 nucleotides. In some embodiments, the tracrRNA comprises 10-190, 10-180, 10-170, 10-160, 10-150, 10-140, 10-130, 10-120, 10-110, 10-100, 10-90, 10-80, 10-70, 10-60, 10-50, 10-40, 10-30, 10-20, 20-100, 30-100, 40-100, 20-90, 20-80, 20-70, 20-60, 20-50, or 30-50 nucleotides. In some embodiments, the tracrRNA comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides.

In some embodiments, the guide polynucleotide is the guide polynucleotide as described herein.

In some embodiments, the target nucleic acid is DNA or RNA. In some embodiments, dsDNA or ssDNA.

In some embodiments, the DNA is the eukaryotic DNA. In some embodiments, the eukaryotic DNA is non-human mammalian DNA, non-human primate DNA, human DNA, plant DNA, insect DNA, bird DNA, reptile DNA, rodent DNA, fish DNA, worm/nematode DNA, or yeast DNA.

In some embodiments, the target nucleic acid is a disease or a condition-related gene or a signaling biochemical pathway-related gene, or the target nucleic acid is a reporter gene. For example, the disease or disorder is a hematologic disease or disorder, an ophthalmic disease or disorder, a neurological disease or disorder, a respiratory disease or disorder, a hepatic disease or disorder, a metabolic disease or disorder, a cancer, or an infectious disease.

In some embodiments, the target nucleic acid is a gene as listed in Table 27.

In some embodiments, the target nucleic acid is a disease or disorder related gene, the disease or disorder being selected from: hemophilia A, Best yolk-like macular dystrophy, B-cell acute lymphoblastic leukemia, hemophilia B, CDKL5 deficiency, CLN2 disease, Niemann-Pick disease type C, Dravet syndrome, FOXGI syndrome, GM1ganglioside storage disease, GM2 ganglioside deposition disease, HIV infection, HSV infection, Usher syndrome type IB, Usher syndrome type IIA, Mucopolysaccharidosis type IIIA, Mucopolysaccharidosis type IIIB, Gaucher disease type III, Mucopolysaccharidosis type II, type II diabetes, Mucopolysaccharidosis type IV, Gaucher disease type I, Mucopolysaccharidosis type I, type I diabetes, Usher syndrome type I, KCNQ2 epileptic encephalopathy, Leber hereditary optic neuropathy, Leigh syndrome, Prader-Willi syndrome, SLC13A5 deficiency, X-linked myotubular myopathy, X-linked retinoschisis, X-linked retinitis pigmentosa, α1-antitrypsin deficiency, α-mannoside storage disease, α-thalassemia, β-thalassemia, Alzheimer's disease, Bardet-Biedl syndrome, white dot retinal degeneration, leukocyte adhesion deficiency type I, galactosemia, bladder cancer, overactive bladder, phenylketonuria, nasopharyngeal carcinoma, Bietti's crystalline dystrophy, pyruvate kinase deficiency, erectile dysfunction, autosomal recessive congenital ichthyosis, adult glucan body disease, traumatic arthritis, homozygous familial hypercholesterolemia, Fragile X syndrome, thalassemia, hypophosphatasia, epilepsy, multiple myeloma, multiple system atrophy, frontotemporal dementia, catecholamine-sensitive polymorphic ventricular tachycardia, Fabry's disease, Fanconi's anemia, aromatic L-amino acid decarboxylase deficiency, radiation-induced xerostomia, non-Hodgkin's lymphoma, non-muscle invasive bladder carcinoma, non-alcoholic fatty liver disease, non-small cell lung cancer, hypertrophic cardiomyopathy, hypertrophic scar, obesity, peroneal muscular dystrophy type 1A, peroneal muscular dystrophy type 2A, pulmonary hypertension, Friedrich's ataxia, peritoneal carcinoma, liver cancer, hepatocellular carcinoma, dry age-related macular degeneration, sicca syndrome, hyperuricemia, hyperlipidemia, Gaucher disease, autism spectrum disorders, osteoarthritis, bone marrow failure syndromes, citrullinemia type I, coronary heart disease, cystinosis, melanoma, Huntington's disease, amyotrophic lateral sclerosis, urge incontinence, acute intermittent porphyria, acute lymphoblastic leukemia, spinal cerebellar ataxia, spinal muscular atrophy with respiratory distress type 1, spinal muscular atrophy, Tay-Sachs disease, methylmalonic acidemia, thyroid carcinoma, pseudohypertrophic muscular dystrophy, anaplastic astrocytoma, intermittent claudication, junctional epidermolysis bullosa, glioma, glioblastoma, corneal graft rejection, colorectal cancer, progressive multifocal leukoencephalopathy, progressive familial intrahepatic cholestasis, giant-axonal neuropathy, Canavan's disease, cocaine addiction, Klaber's disease, Kriegler-Najjar syndrome, oral cancer, Angelman syndrome, diffuse intrinsic pontine glioma, Lafora's disease, rheumatoid arthritis, sickle cell disease, lymphedema, ovarian cancer, chronic lymphocytic leukemia, chronic granulomatous disease, chronic nephrogenic anemia, chronic pain, chronic hepatitis B, Menkes' disease, cystic fibrosis, Netherseton's syndrome, ornithine transcarbamylase deficiency, Parkinson's disease, Pompe's disease, uveitis, prostate cancer, vestibular schwannoma, ankylosing muscular dystrophy, ankylosing spondylitis, castration-resistant prostate cancer, glaucoma, achromatopsia, ischemic heart failure, lysosomal storage disease, sarcoma, breast cancer, Rett's syndrome, triple-negative breast cancer, Sandhoff's disease, color blindness, heart failure with reduced ejection fraction, neuronal ceroid lipofuscinosis, adrenoleukodystrophy, renal cell carcinoma, wet age-related macular degeneration, eczema, thrombocytopenia with immunodeficiency syndrome, esophageal cancer, optic neuropathy, optic nerve atrophy, retinal vein occlusion, retinitis pigmentosa, rhodopsin-mediated autosomal dominant retinitis pigmentosa, ependymoma, fallopian tube carcinoma, bilateral vestibulopathies, Stargardt's disease, diabetic macular edema, diabetic neuropathy, diabetic retinopathy, diabetic peripheral neuralgia, diabetic foot, glycogenosis, glycogenosis type Ia, glycogenosis type IIb, atopic dermatitis, hearing loss, hearing impairment, head and neck cancer, squamous cell carcinoma of the head and neck, Wilson's disease, stable angina pectoris, Usher's syndrome, choroideremia, Leber's congenital amaurosis, congenital adrenal hyperplasia, cardiomyopathy, angina pectoris, heart failure, COVID-19 infection, pleural mesothelioma, acne vulgaris, severe combined immunodeficiency diseases, severe limb ischemia, oculopharyngeal muscular dystrophy, pancreatic cancer, graft-versus-host disease, hereditary retinal dystrophy, hereditary angioedema, hepatitis B, heterotrophic cerebral leukoencephalic dystrophy, psoriatic arthritis, recessive genetic dystrophic epidermolysis bullosa, infantile malignant osteosclerosis, dystrophic epidermolysis bullosa, morphea, primary immune deficiency, heterozygous familial hypercholesterolemia, limb-girdle muscular dystrophy type 2B, limb-girdle muscular dystrophy type 2C, limb-girdle muscular dystrophy type 2D, limb-girdle muscular dystrophy type 2E, limb-girdle muscular dystrophy type 2I, limb-girdle muscular dystrophy type 2L, limb ischemic disease, lipoprotein lipase deficiency, severe congenital neutrophilic dysphoria, wrinkles, stroke, sciatica, schizophrenia, depression, drug addiction, autism, idiopathic pulmonary fibrosis, hyperlipidemia, transthyretin (ATTR) amyloidosis, alpha-1-antitrypsin deficiency (AATD) liver disease, and AATD lung disease.

In some embodiments, genes associated with ATTR amyloidosis comprise, but are not limited to, ATTR.

Genes associated with Leber hereditary optic neuropathy comprise, but are not limited to, MT-ND4.

Genes associated with the AATD liver disease comprise, but are not limited to, AATD.

Genes associated with the AATD lung disease comprise, but are not limited to, AATD.

Genes associated with the graft-versus-host disease comprise, but are not limited to, thymidine kinase genes.

Genes associated with hereditary retinal dystrophy comprise, but are not limited to, RPE65.

Genes associated with spinal muscular atrophy comprise, but are not limited to, SMN1.

Genes associated with osteoarthritis comprise, but are not limited to, TGF-β1.

Genes associated with hemophilia A comprise, but are not limited to, factor VIII.

Genes associated with hemophilia B comprise, but are not limited to, factor IX.

Genes associated with cystic fibrosis comprise, but are not limited to, CFTR.

Genes associated with Parkinson's disease comprise, but are not limited to, Gad1, Gad2, PTBP1, KEAP1, RE1, Amigo1, Gprc5c, Let-7a, Pnky, LRRK2, SNCA, GBA, miR-92b, miR-9, miR-124, miR-181, HMGB1, TRIM72, GPNMB, and REST.

Genes associated with Usher syndrome comprise, but are not limited to, USH2A.

Genes associated with α-thalassemia, β-thalassemia, and sickle cell disease comprise, but are not limited to, BCL11A, HBG, HBA, and HBB.

Genes associated with pulmonary hypertension comprise, but are not limited to, eNOS.

Genes associated with Stargardt's disease comprise, but are not limited to, ABCA4.

Genes associated with age-related macular degeneration comprise, but are not limited to, VEGFA, VEGFR, IL17, Kir7.1, LCN-2, IRAK-M, CD59, LTA4H, GPX4, GLS1, PAPP-A, cGAS, STING, mTOR, GCN2, Nrf2, Ang 2, CTGF, complement C3, complement C5, CHFR4b, DOCK6, CTSS, ELN, and FGF2.

Genes associated with glaucoma comprise, but are not limited to, AQP1, ADRB2, NMNTA2, NRP1, Hrh1, Anxa2, OPA1, Cx43, ANGPTL7, MYOC, ROCK1, ROCK2, TIMP1, TIMP2, TIMP3, TIMP4, carbonic anhydrase CA2, carbonic anhydrase CA4, and carbonic anhydrase CA12.

Genes associated with idiopathic pulmonary fibrosis comprise, but are not limited to, CTGF.

Genes associated with hyperlipidemia comprise, but are not limited to, PCSK9.

Genes associated with Alzheimer's disease comprise, but are not limited to, NGF.

Genes associated with coronary heart disease comprise, but are not limited to, VEGFA and bFGF.

Genes associated with chronic nephrogenic anemia comprise, but are not limited to, EPO.

Genes associated with congenital amaurosis comprise, but are not limited to, RPE65.

Genes associated with retinitis pigmentosa comprise, but are not limited to, PDE6B.

Genes associated with phenylketonuria comprise, but are not limited to, PAH.

Genes associated with epilepsy comprise, but are not limited to, GAT1.

Some embodiments of the present disclosure provide a vector system. The vector system comprises one or more recombinant vectors. The recombinant vectors comprise the isolated nucleic acid or the CRISPR-Cas12 system as described herein.

In some embodiments, the recombinant vector further comprises a regulatory sequence.

In some embodiments, the vector system comprises one or more recombinant vectors comprising a polynucleotide sequence encoding the Cas12 protein, the inactivated Cas12 mutant, or the fusion proteins or conjugate as described herein and a polynucleotide sequence encoding the guide polynucleotide.

In some embodiments, the polynucleotide sequence encoding the Cas12 protein, the inactivated Cas12 mutant, or the fusion protein or conjugate is operably linked to the regulatory sequence 1.

In some embodiments, the polynucleotide sequence encoding the guide polynucleotide is operably linked to the regulatory sequence 2.

Further, in some embodiments, the regulatory sequence 1 is the same as or different from the regulatory sequence 2.

In some embodiments, the regulatory sequence is optionally selected from one or more of: a promoter, an enhancer, an internal ribosome entry site, and a transcription termination signal. The promoter comprises a constitutive promoter, an inducible promoter, a broad-spectrum promoter, or a tissue-specific promoter, and/or the transcriptional termination signal comprises a polyadenylation signal or a poly-U sequence.

In some embodiments, a scaffold of the one or more recombinant vectors is an adeno-associated virus vector, a lentiviral vector, or a virus-like particle.

In some embodiment of the present disclosure, when the scaffold is the adeno-associated virus vector, the adeno-associated virus vector is a recombinant adeno-associated virus vector of serotype AAV1, AAV2, AAV4, AAV5, AAV6, AAV7, AAVrh74, AAV8, AAV9, AAV10, AAV11, AAV12, or AAV13; when the scaffold is the lentiviral vector, the lentiviral vector is pseudotyped with an envelope protein; in some embodiments, the isolated nucleic acid is linked to an aptamer sequence; and when the scaffold is the virus-like particle, the isolated nucleic acid is linked to a gene encoding a gag protein.

Some embodiments of the present disclosure provide a delivery system. The delivery system comprises (1) a delivery tool, and (2) the Cas12 protein, the guide polynucleotide, the inactivated Cas12 mutant, the fusion protein or conjugate, the isolated nucleic acid, the CRISPR-Cas12 system, or the vector system as described herein.

In some embodiments, the delivery tool is a virus, a lipid nanoparticle, a nanoparticle, a liposome, an exosome, a microbubble, or a gene gun.

In some embodiments, the delivery tool is the lipid nanoparticle comprising the guide polynucleotide and mRNA encoding the Cas12 protein, the inactivated Cas12 mutant, or the fusion protein or conjugate.

Some embodiments of the present disclosure provide a cell comprising the Cas12 protein, the guide polynucleotide, the inactivated Cas12 mutant, the fusion protein or conjugate, the isolated nucleic acid, the CRISPR-Cas12 system, or the vector system as described herein.

In some embodiments of the present disclosure, the cell is a prokaryotic cell.

In some embodiments of the present disclosure, the cell is a eukaryotic cell.

In some embodiments of the present disclosure, the eukaryotic cell is a mammalian cell.

Some embodiments of the present disclosure provide a pharmaceutical composition, wherein the pharmaceutical composition comprises the Cas12 protein, the guide polynucleotide, the inactivated Cas12 mutant, the fusion protein or conjugate, the nucleic acid, the CRISPR-Cas12 system, the vector system, the delivery system, or the cell as described herein.

In some embodiments, the pharmaceutical composition further comprises pharmaceutically acceptable excipients.

Some embodiments of the present disclosure provide a kit, wherein the kit comprises the Cas12 protein, the guide polynucleotide, the inactivated Cas12 mutant, the fusion protein or conjugate, the isolated nucleic acid, the CRISPR-Cas12 system, the vector system, the delivery system, or the cell as described herein.

In some embodiments, the kit further comprises a cut buffer. The cut buffer is any buffer known in the art suitable for cleaving the target nucleic acid by the Cas12 protein.

Some embodiments of the present disclosure provide a use of the Cas12 protein, the guide polynucleotide, the inactivated Cas12 mutant, the fusion protein or conjugate, the nucleic acid, the CRISPR-Cas12 system, the vector system, the delivery system, the cell, the pharmaceutical composition, or the kit as described herein in preparing a reagent or medicament for diagnosing, treating, or preventing a disease or disorder associated with a target nucleic acid.

In some embodiments, the disease or disorder is a hematologic disease or disorder, an ophthalmic disease or disorder, a neurological disease or disorder, a respiratory disease or disorder, a hepatic disease or disorder, a metabolic disease or disorder, a cancer, or an infectious disease. In some embodiments, the reagent or medicament is used to: cleave one or more target nucleic acid molecules or introduce nicks into the one or more target nucleic acid molecules, activate or upregulate an expression of the one or more target nucleic acid molecules, activate or inhibit transcription of the one or more target nucleic acid molecules, inactivate the one or more target nucleic acid molecules, visualize, label, or detect the one or more target nucleic acid molecules, bind the one or more target nucleic acid molecules, transport the one or more target nucleic acid molecules, and mask the one or more target nucleic acid molecules.

In some embodiments, the target nucleic acid is optionally selected from the genes as listed in Table 27, and the disease or disorder is the disease or disorder as listed in Table 27.

In some embodiments, the disease or disorder is selected from: hemophilia A, Best yolk-like macular dystrophy, B-cell acute lymphoblastic leukemia, hemophilia B, CDKL5deficiency, CLN2 disease, Niemann-Pick disease type C, Dravet syndrome, FOXG1 syndrome, GM1 ganglioside storage disease, GM2 ganglioside deposition disease, HIV infection, HSV infection, Usher syndrome type IB, Usher syndrome type IIA, Mucopolysaccharidosis type IIIA, Mucopolysaccharidosis type IIIB, Gaucher disease type III, Mucopolysaccharidosis type II, type II diabetes, Mucopolysaccharidosis type IV, Gaucher disease type I, Mucopolysaccharidosis type I, type I diabetes, Usher syndrome type I, KCNQ2 epileptic encephalopathy, Leber hereditary optic neuropathy, Leigh syndrome, Prader-Willi syndrome, SLC13A5deficiency, X-linked myotubular myopathy, X-linked retinoschisis, X-linked retinitis pigmentosa, α1-antitrypsin deficiency, α-mannoside storage disease, α-thalassemia, β-thalassemia, Alzheimer's disease, Bardet-Biedl syndrome, white dot retinal degeneration, leukocyte adhesion deficiency type I, galactosemia, bladder cancer, overactive bladder, phenylketonuria, nasopharyngeal carcinoma, Bietti's crystalline dystrophy, pyruvate kinase deficiency, erectile dysfunction, autosomal recessive congenital ichthyosis, adult glucan body disease, traumatic arthritis, homozygous familial hypercholesterolemia, Fragile X syndrome, thalassemia, hypophosphatasia, epilepsy, multiple myeloma, multiple system atrophy, frontotemporal dementia, catecholamine-sensitive polymorphic ventricular tachycardia, Fabry's disease, Fanconi's anemia, aromatic L-amino acid decarboxylase deficiency, radiation-induced xerostomia, non-Hodgkin's lymphoma, non-muscle invasive bladder carcinoma, non-alcoholic fatty liver disease, non-small cell lung cancer, hypertrophic cardiomyopathy, hypertrophic scar, obesity, peroneal muscular dystrophy type 1A, peroneal muscular dystrophy type 2A, pulmonary hypertension, Friedrich's ataxia, peritoneal carcinoma, liver cancer, hepatocellular carcinoma, dry age-related macular degeneration, sicca syndrome, hyperuricemia, hyperlipidemia, Gaucher disease, autism spectrum disorders, osteoarthritis, bone marrow failure syndromes, citrullinemia type I, coronary heart disease, cystinosis, melanoma, Huntington's disease, amyotrophic lateral sclerosis, urge incontinence, acute intermittent porphyria, acute lymphoblastic leukemia, spinal cerebellar ataxia, spinal muscular atrophy with respiratory distress type 1, spinal muscular atrophy, Tay-Sachs disease, methylmalonic acidemia, thyroid carcinoma, pseudohypertrophic muscular dystrophy, anaplastic astrocytoma, intermittent claudication, junctional epidermolysis bullosa, glioma, glioblastoma, corneal graft rejection, colorectal cancer, progressive multifocal leukoencephalopathy, progressive familial intrahepatic cholestasis, giant-axonal neuropathy, Canavan's disease, cocaine addiction, Klaber's disease, Kriegler-Najjar syndrome, oral cancer, Angelman syndrome, diffuse intrinsic pontine glioma, Lafora's disease, rheumatoid arthritis, sickle cell disease, lymphedema, ovarian cancer, chronic lymphocytic leukemia, chronic granulomatous disease, chronic nephrogenic anemia, chronic pain, chronic hepatitis B, Menkes' disease, cystic fibrosis, Netherseton's syndrome, ornithine transcarbamylase deficiency, Parkinson's disease, Pompe's disease, uveitis, prostate cancer, vestibular schwannoma, ankylosing muscular dystrophy, ankylosing spondylitis, castration-resistant prostate cancer, glaucoma, achromatopsia, ischemic heart failure, lysosomal storage disease, sarcoma, breast cancer, Rett's syndrome, triple-negative breast cancer, Sandhoff's disease, color blindness, heart failure with reduced ejection fraction, neuronal ceroid lipofuscinosis, adrenoleukodystrophy, renal cell carcinoma, wet age-related macular degeneration, eczema, thrombocytopenia with immunodeficiency syndrome, esophageal cancer, optic neuropathy, optic nerve atrophy, retinal vein occlusion, retinitis pigmentosa, rhodopsin-mediated autosomal dominant retinitis pigmentosa, ependymoma, fallopian tube carcinoma, bilateral vestibulopathies, Stargardt's disease, diabetic macular edema, diabetic neuropathy, diabetic retinopathy, diabetic peripheral neuralgia, diabetic foot, glycogenosis, glycogenosis type Ia, glycogenosis type IIb, atopic dermatitis, hearing loss, hearing impairment, head and neck cancer, squamous cell carcinoma of the head and neck, Wilson's disease, stable angina pectoris, Usher's syndrome, choroideremia, Leber's congenital amaurosis, congenital adrenal hyperplasia, cardiomyopathy, angina pectoris, heart failure, COVID-19 infection, pleural mesothelioma, acne vulgaris, severe combined immunodeficiency diseases, severe limb ischemia, oculopharyngeal muscular dystrophy, pancreatic cancer, graft-versus-host disease, hereditary retinal dystrophy, hereditary angioedema, hepatitis B, heterotrophic cerebral leukoencephalic dystrophy, psoriatic arthritis, recessive genetic dystrophic epidermolysis bullosa, infantile malignant osteosclerosis, dystrophic epidermolysis bullosa, morphea, primary immune deficiency, heterozygous familial hypercholesterolemia, limb-girdle muscular dystrophy type 2B, limb-girdle muscular dystrophy type 2C, limb-girdle muscular dystrophy type 2D, limb-girdle muscular dystrophy type 2E, limb-girdle muscular dystrophy type 2I, limb-girdle muscular dystrophy type 2L, limb ischemic disease, lipoprotein lipase deficiency, severe congenital neutrophilic dysphoria, wrinkles, stroke, sciatica, schizophrenia, depression, drug addiction, autism, idiopathic pulmonary fibrosis, hyperlipidemia, transthyretin (ATTR) amyloidosis, alpha-1-antitrypsin deficiency (AATD) liver disease, and AATD lung disease.

In some embodiments, genes associated with the ATTR amyloidosis comprise, but is not limited to, ATTR.

Genes associated with the Leber hereditary optic neuropathy comprise, but are not limited to, MT-ND4.

Genes associated with the AATD liver disease comprise, but are not limited to, AATD.

the genes associated with the AATD lung disease comprise, but are not limited to, AATD.

Genes associated with the graft-versus-host disease comprise, but are not limited to, thymidine kinase genes.

Genes associated with the hereditary retinal dystrophy comprise, but are not limited to, RPE65.

Genes associated with the spinal muscular atrophy comprise, but are not limited to, SMN1.

Genes associated with the osteoarthritis comprise, but are not limited to, TGF-β1.

Genes associated with the hemophilia A comprise, but are not limited to, factor VIII.

Genes associated with the hemophilia B comprise, but are not limited to, factor IX.

Genes associated with the cystic fibrosis comprise, but are not limited to, CFTR.

Genes associated with the Parkinson's disease comprise, but are not limited to, Gad1, Gad2, PTBP1, KEAP1, RE1, Amigo1, Gprc5c, Let-7a, Pnky, LRRK2, SNCA, GBA gene, miR-92b, miR-9, miR-124, miR-181, HMGB1, TRIM72, GPNMB, and REST.

Genes associated with Usher syndrome comprise, but are not limited to, USH2A.

Genes associated with α-thalassemia, β-thalassemia, and sickle cell disease comprise, but are not limited to, BCL11A, HBG, HBA, and HBB.

Genes associated with the pulmonary hypertension comprise, but are not limited to, eNOS.

Genes associated with the Stargardt's disease comprise, but are not limited to, ABCA4.

Genes associated with the age-related macular degeneration comprise, but are not limited to, VEGFA, VEGFR, IL17, Kir7.1, LCN-2, IRAK-M, CD59, LTA4H, GPX4, GLS1, PAPP-A, cGAS, STING, mTOR, GCN2, Nrf2, Ang 2, CTGF, complement C3, complement C5, CHFR4b, DOCK6, CTSS, ELN, and FGF2.

Genes associated with the glaucoma comprise, but are not limited to, AQP1, ADRB2, NMNTA2, NRP1, Hrh1, Anxa2, OPA1, Cx43, ANGPTL7, MYOC, ROCK, ROCK2, TIMP1, TIMP2, TIMP3, TIMP4, carbonic anhydrase CA2, carbonic anhydrase CA4, and carbonic anhydrase CA12.

Genes associated with the idiopathic pulmonary fibrosis comprise, but are not limited to, CTGF.

Genes associated with cardiovascular diseases such as hyperlipidemia comprise, but are not limited to, PCSK9 (Proprotein Convertase Subtilisin/Kexin Type 9), ANGPTL3 (Angiopoietin Like 3), LPA (Lipoprotein (a)), APOC3 (Apolipoprotein C3), and APOB (Apolipoprotein B). Genes associated with hypertension comprise, but are not limited to, AGT (Angiotensinogen). Genes associated with ATTR comprise, but are not limited to, TTR (Transthyretin). Genes associated with obesity comprise, but are not limited to, INHBE (Inhibin Subunit Beta E).

Genes associated with the Alzheimer's disease comprise, but are not limited to, NGF.

Genes associated with the coronary heart disease comprise, but are not limited to, VEGFA and bFGF.

Genes associated with the chronic nephrogenic anemia comprise, but are not limited to, EPO.

Genes associated with the congenital amaurosis comprise, but are not limited to, RPE65.

Genes associated with the retinitis pigmentosa comprise, but are not limited to, PDE6B.

Genes associated with the phenylketonuria comprise, but are not limited to, PAH.

Genes associated with the epilepsy comprise, but are not limited to, GATI.

Some embodiments of the present disclosure provide a method for detecting, binding, or cleaving a target nucleic acid, comprising: using the Cas12 protein, the guide polynucleotide, the inactivated Cas12 mutant, the fusion protein or conjugate, the nucleic acid, the CRISPR-Cas12 system, the vector system, the delivery system, the cell, the pharmaceutical composition, or the kit as described herein to contact the target nucleic acid.

In some embodiments, the method is for non-diagnostic and/or non-therapeutic purposes; and/or the fusion protein or conjugate comprises a detectable marker, such as a marker detectable by fluorescence, DNA blotting, or FISH.

In some embodiments, when the method is for cleaving the target nucleic acid, the method further comprises performing a cleavage reaction using a cut buffer. The cut buffer may be any buffer known in the art suitable for cleaving the target nucleic acid by the Cas12 protein.

Some embodiments of the present disclosure provide is a method for altering a cell state, comprising using the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit as described herein to contact the cell to alter a cell state.

In some embodiments of the present disclosure, the method results in one or more of: an increase or decrease in an expression of a specific gene, an induction of cellular senescence in vitro or in vivo, an induction of cellular cycle arrest in vitro or in vivo, a cellular growth promotion and/or a cellular growth inhibition in vitro or in vivo, an induction of anergy in vitro or in vivo, an induction of apoptosis in vitro or in vivo, and an induction of necrosis in vitro or in vivo.

In some embodiments, the method is for non-diagnostic and/or non-therapeutic purposes.

Some embodiments of the present disclosure provide a method for diagnosing, treating, or preventing a disease or disorder associated with a target nucleic acid, comprising applying the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit as described herein to a sample from a subject in need or the subject in need.

In some embodiments, the target nucleic acid is optionally selected from genes as listed in Table 27, and the disease or disorder is the disease or disorder as listed in Table 27.

In some embodiments, the disease or disorder is a hematologic disease or disorder, an ophthalmic disease or disorder, a neurological disease or disorder, a respiratory disease or disorder, a hepatic disease or disorder, a metabolic disease or disorder, a cancer, or an infectious disease.

Some embodiments of the present disclosure provide a use of the Cas12 protein, the guide polynucleotide, the inactivated Cas12 mutant, the fusion proteins or conjugate, the isolated nucleic acid, the CRISPR-Cas12 system, the vector system, the delivery system, the cell, the pharmaceutical composition, or the kit as described herein in diagnosing, treating, or preventing a disease or disorder associated with the target nucleic acid.

In some embodiments, the target nucleic acid is optionally selected from genes as listed in Table 27, and the disease or disorder is the disease or disorder as listed in Table 27.

In some embodiments, the disease or disorder is a hematologic disease or disorder, an ophthalmic disease or disorder, a neurological disease or disorder, a respiratory disease or disorder, a hepatic disease or disorder, a metabolic disease or disorder, a cancer, or an infectious disease.

On the basis of conforming to the common knowledge in the field, the above preferred conditions may be arbitrarily combined, thereby obtaining the preferred embodiments of the present disclosure.

### BRIEF DESCRIPTION OF THE DRAWINGS

FIG. 1A is a diagram illustrating an evolutionary relationship of Cas proteins and known Cas12 isoform proteins (performing sequence alignment using MAFFT, then constructing an evolutionary tree using FastTree) according to embodiments of the present disclosure, and FIG. 1B shows the proteins according to some embodiments of the present disclosure;

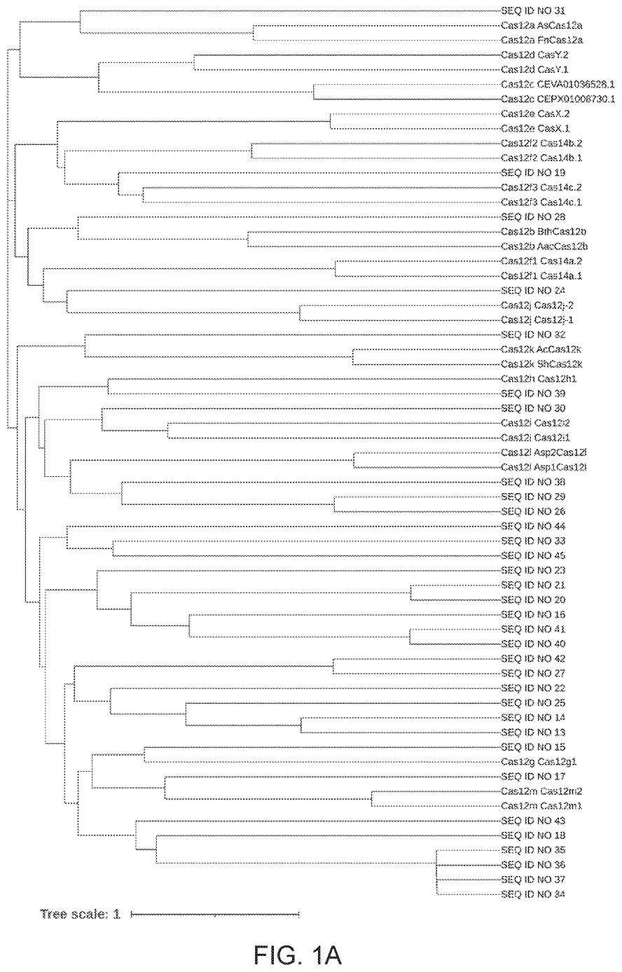

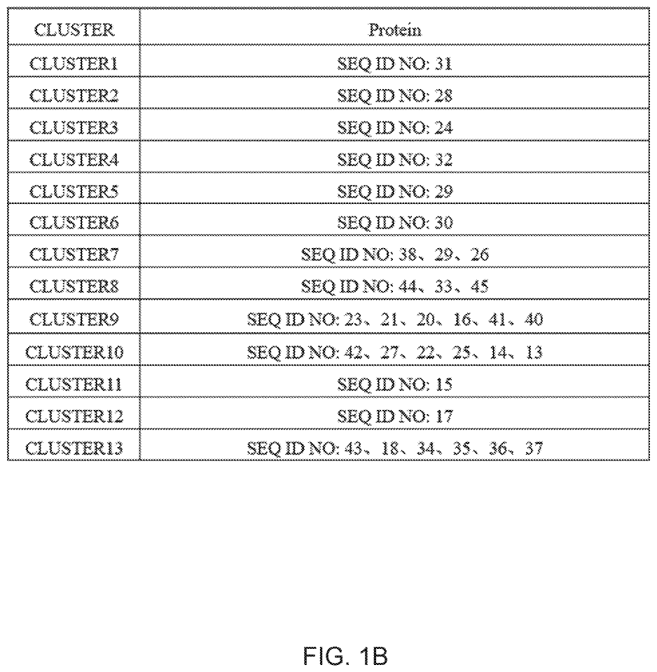

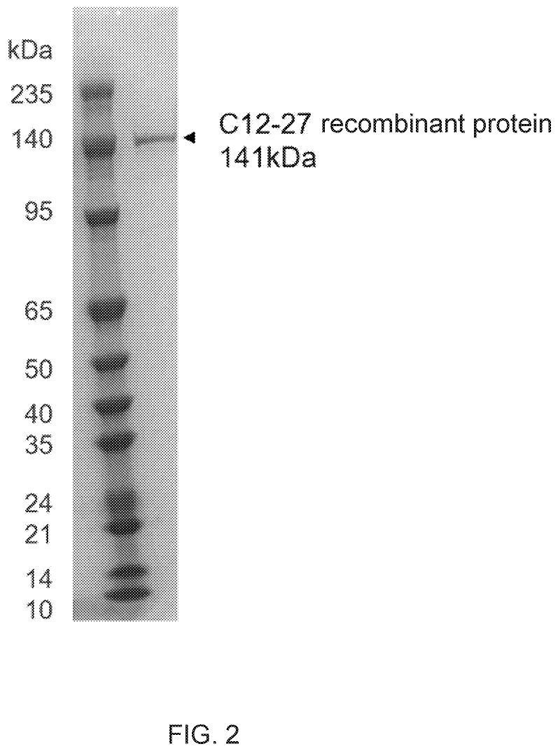

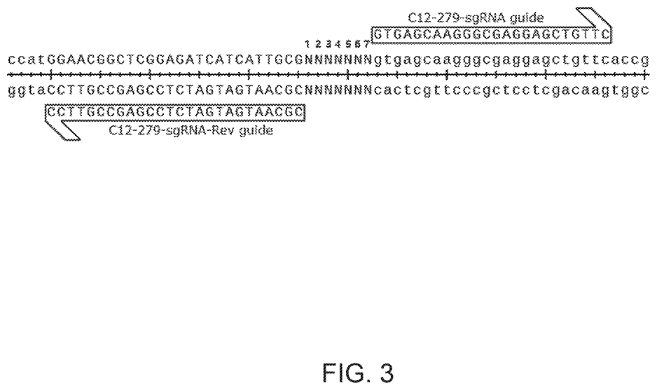

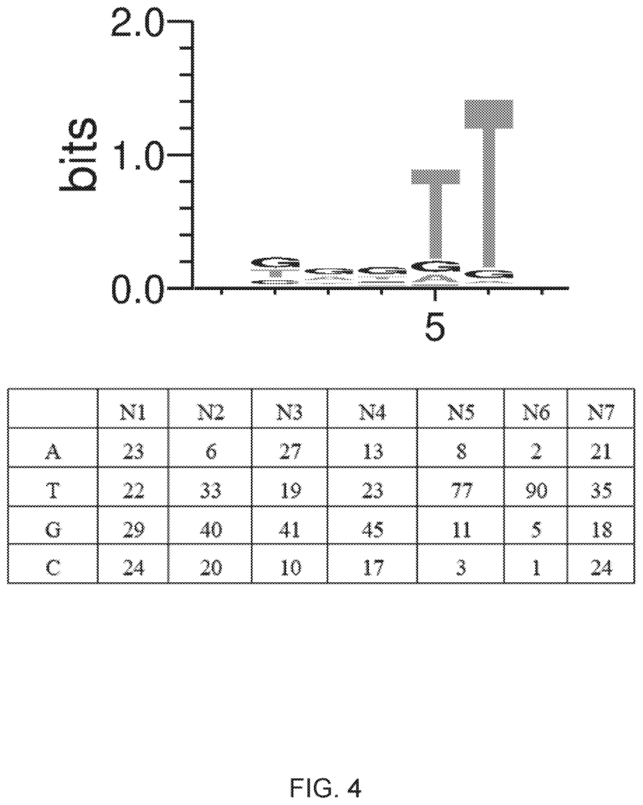

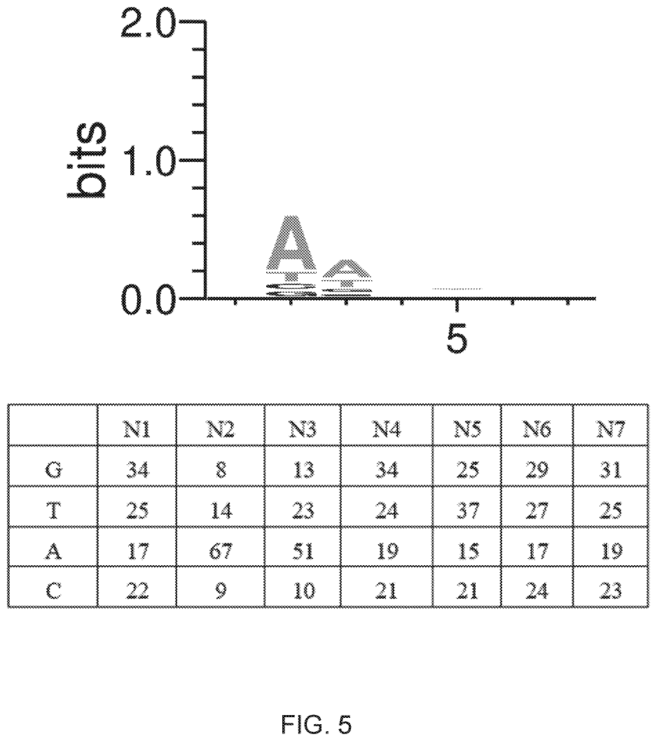

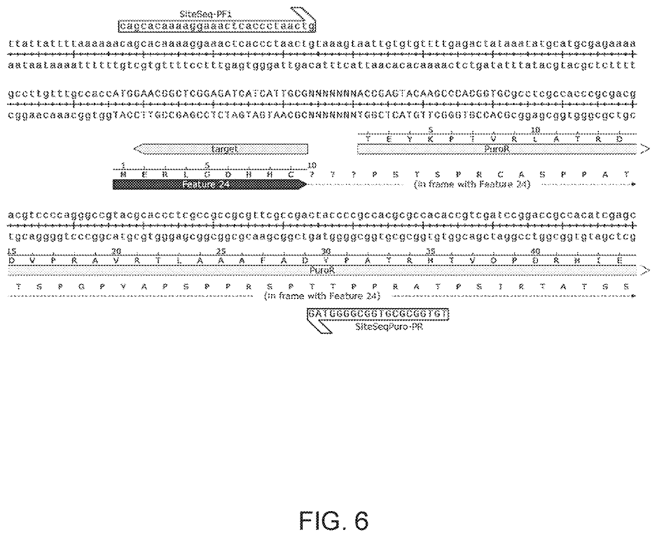

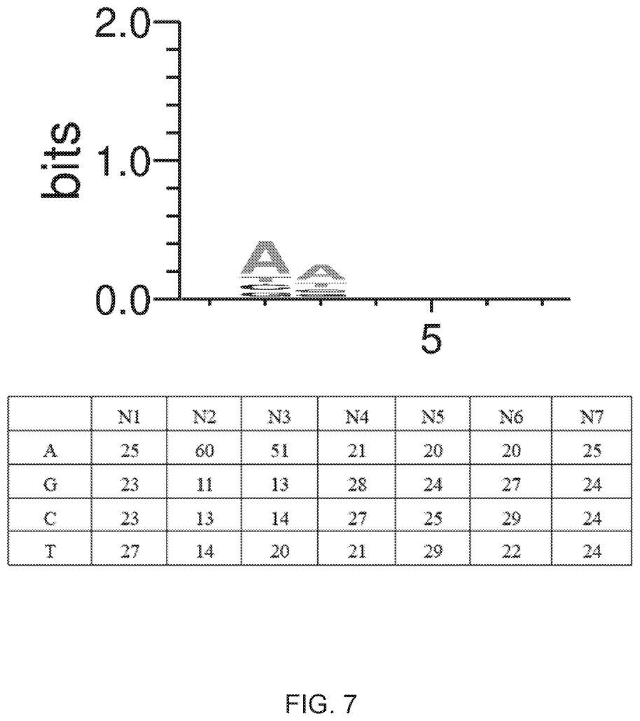

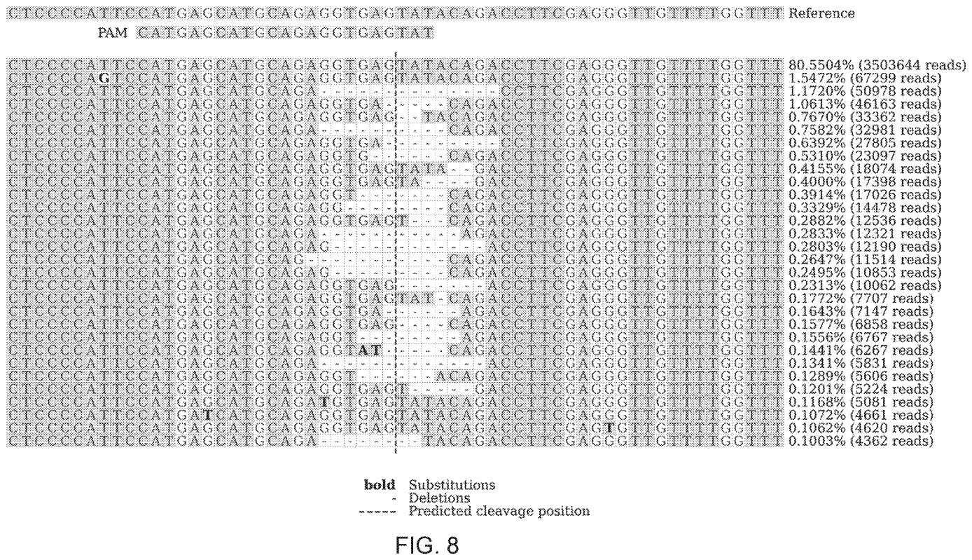

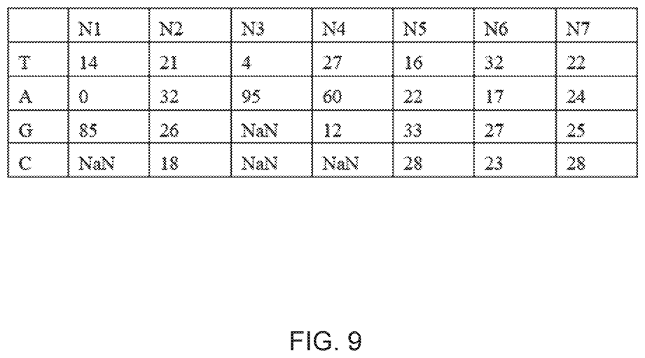

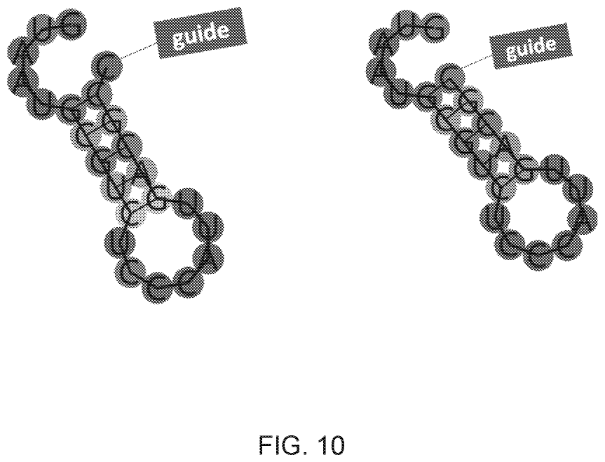

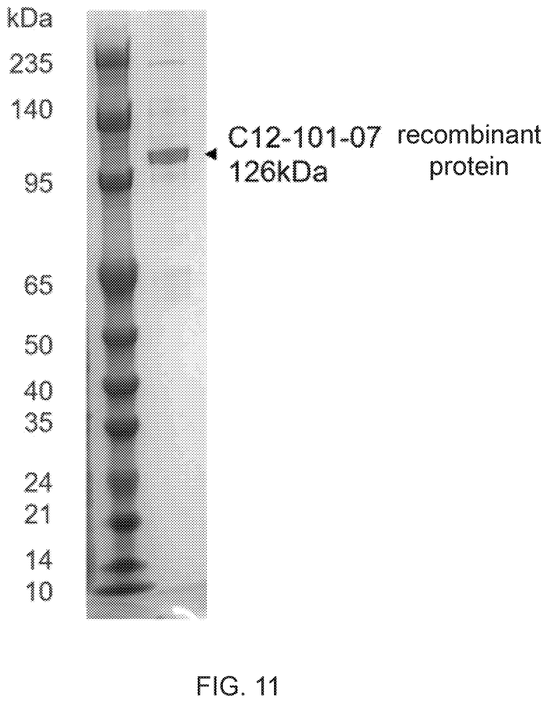

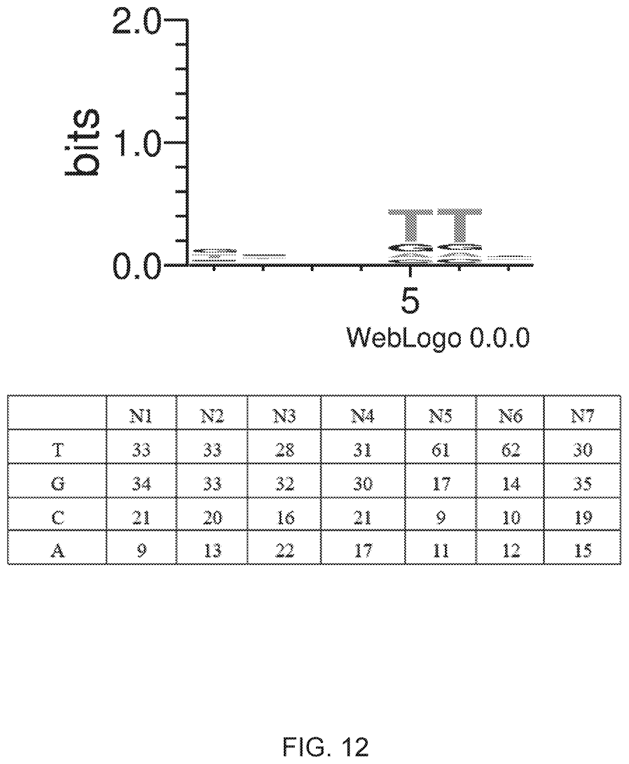

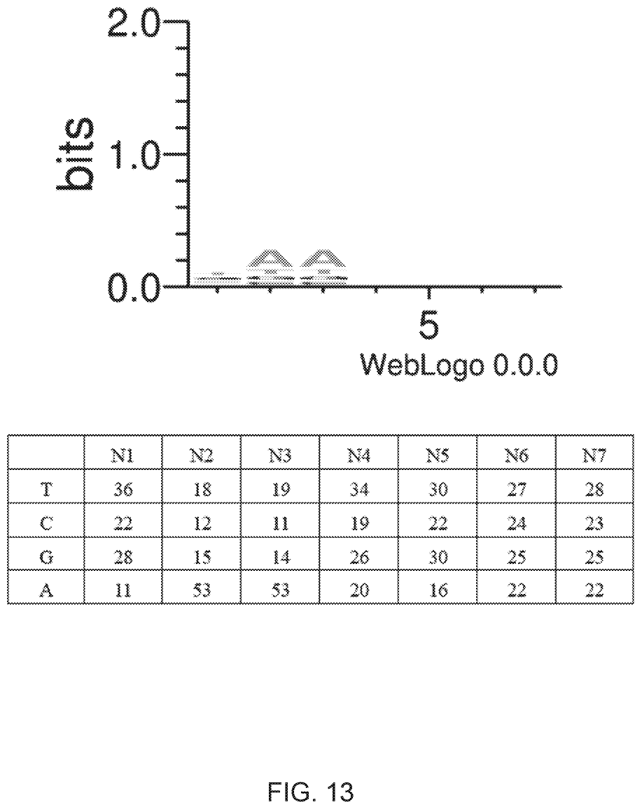

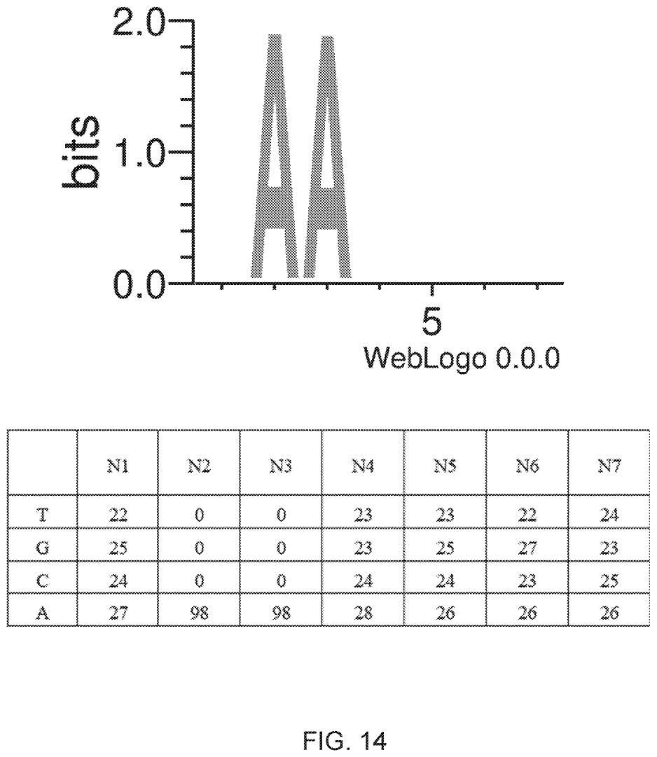

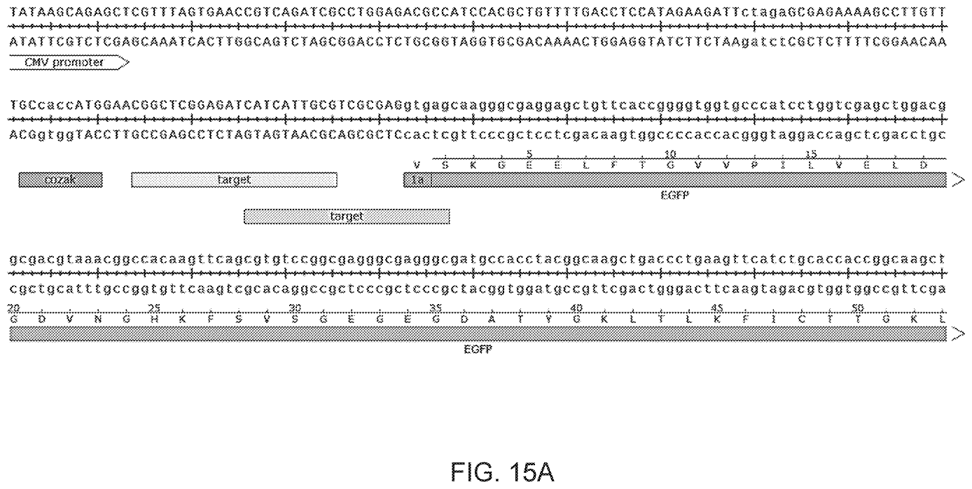

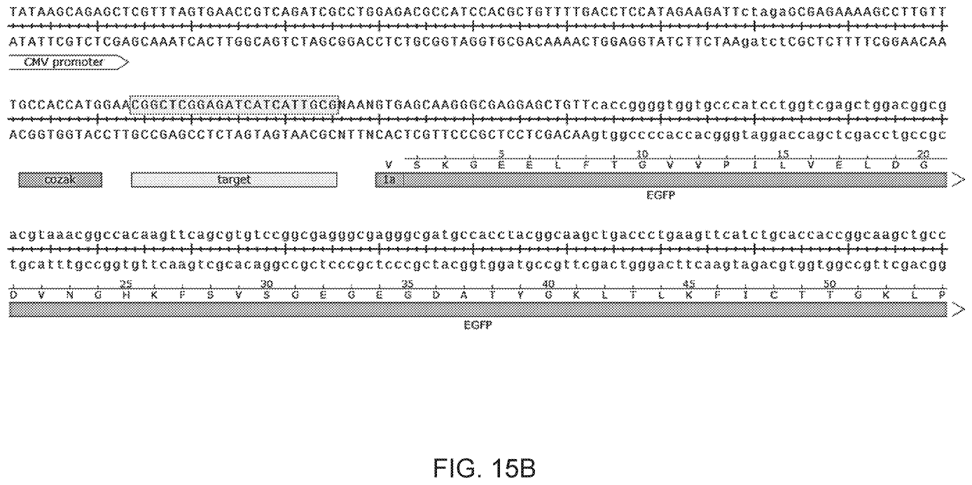

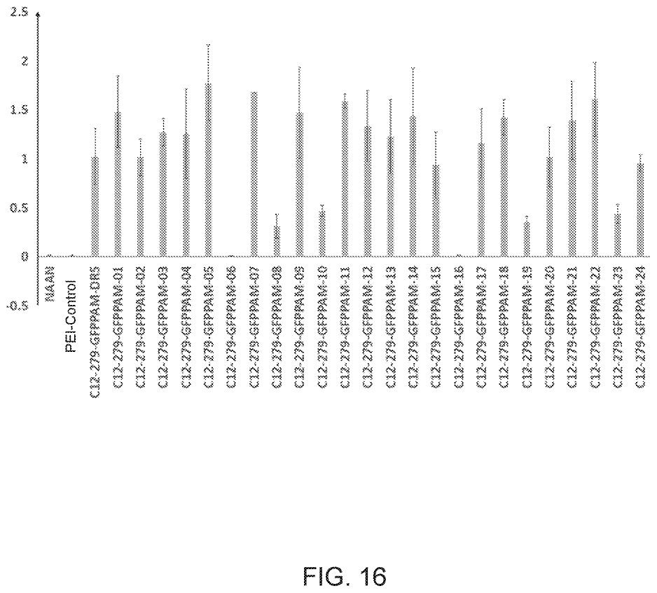

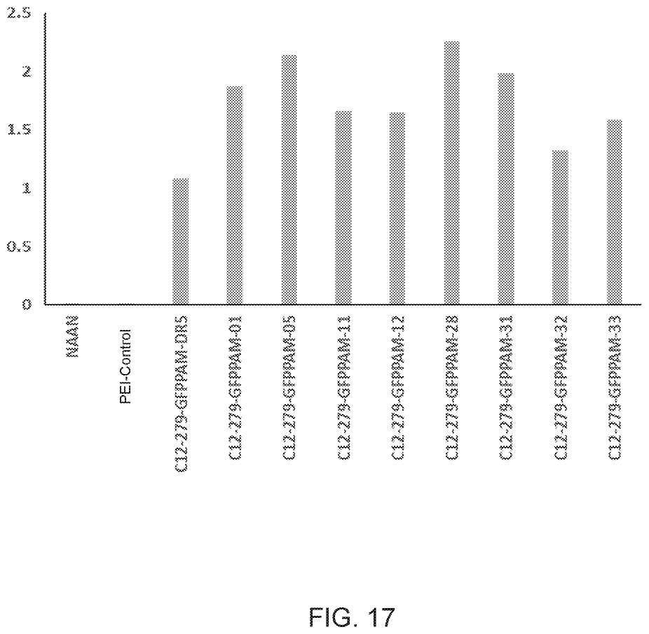

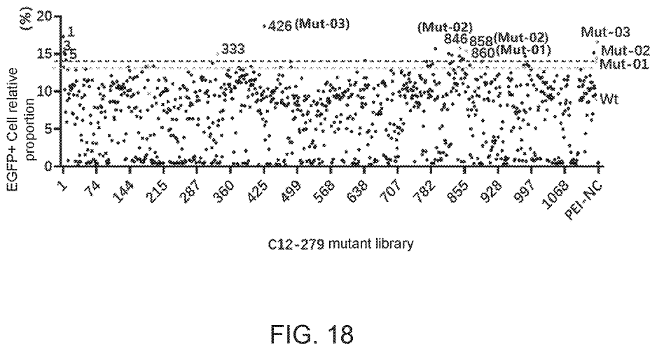

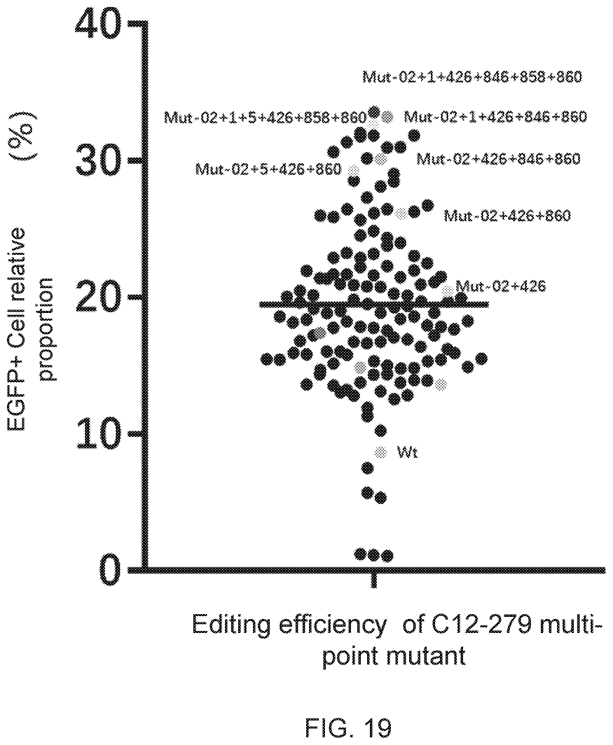

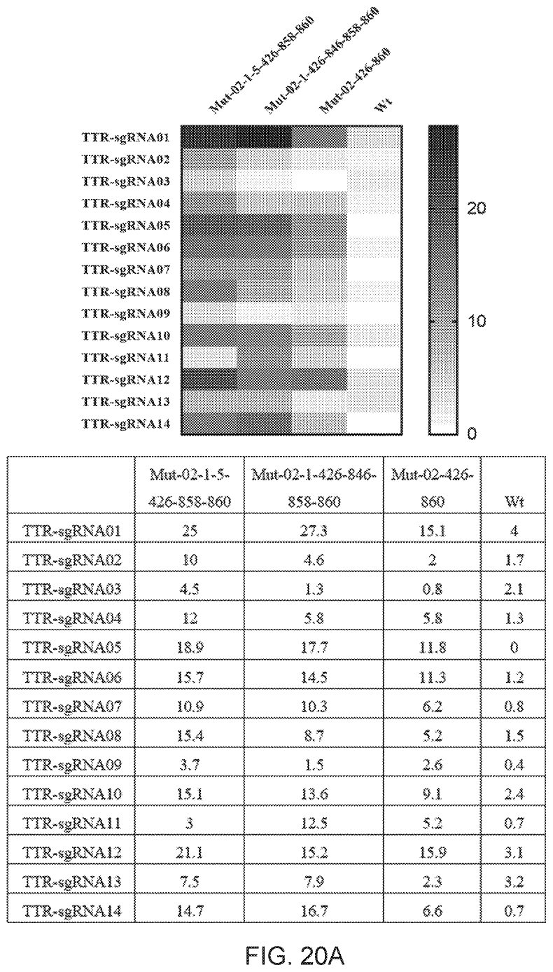

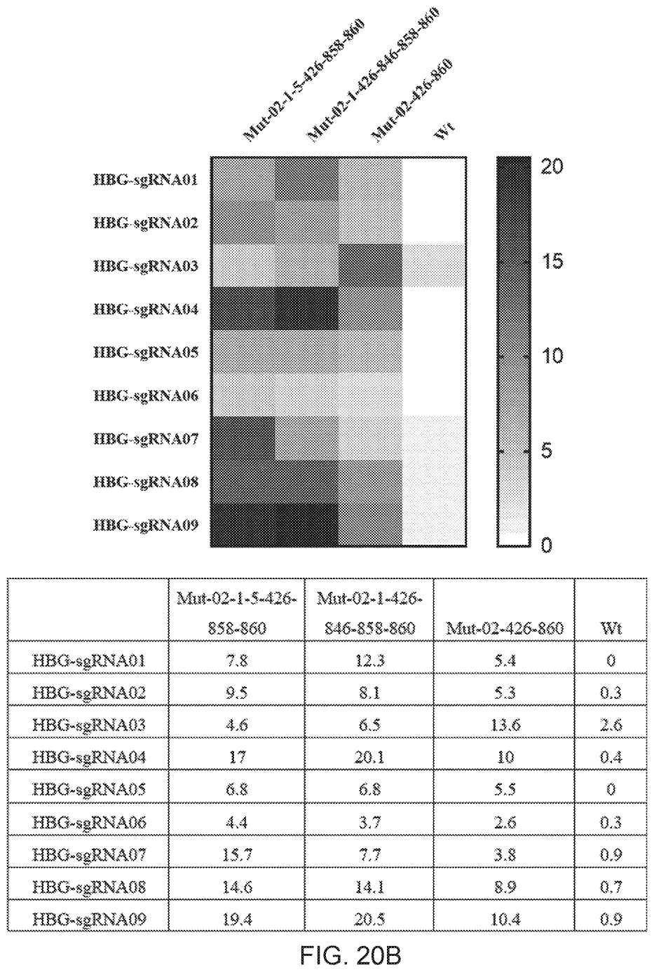

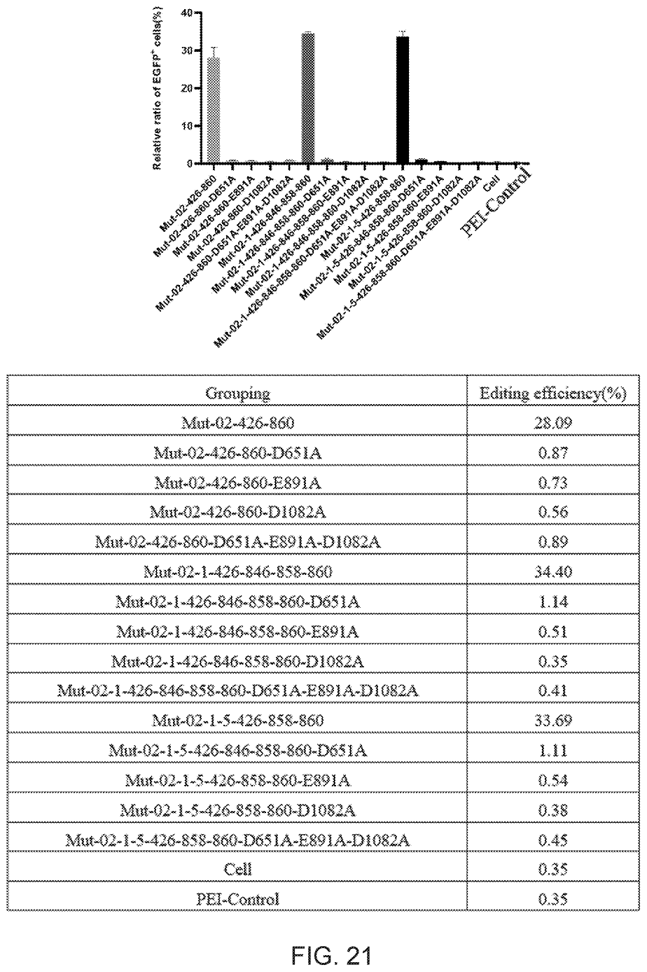

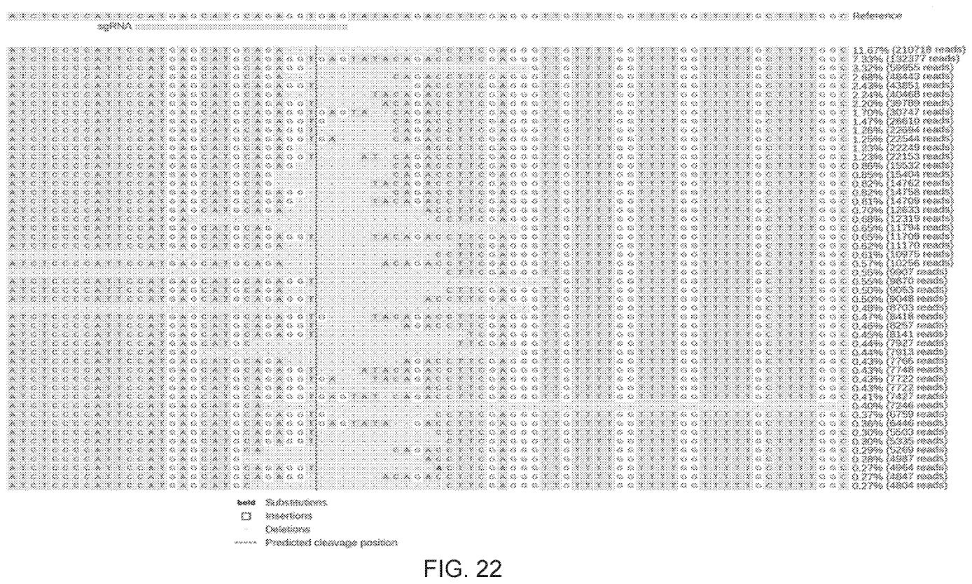

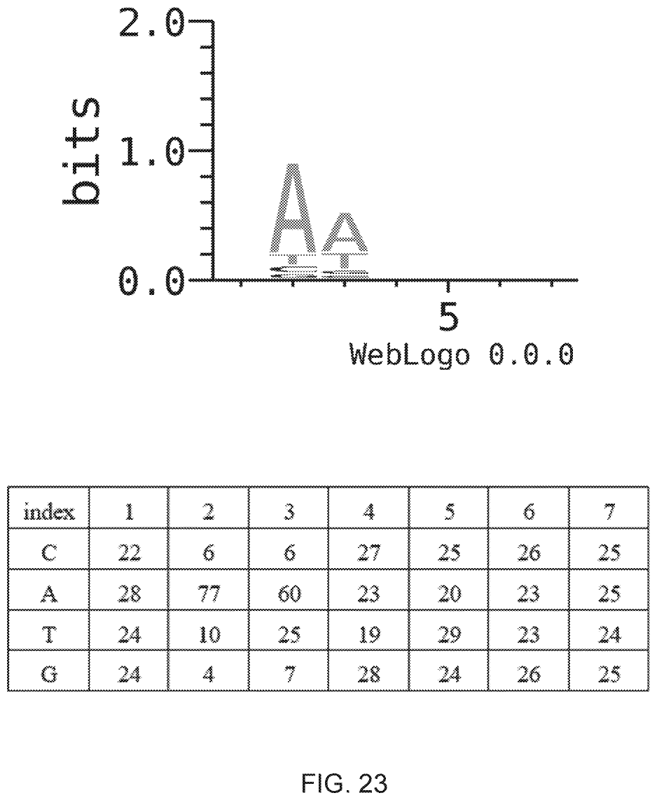

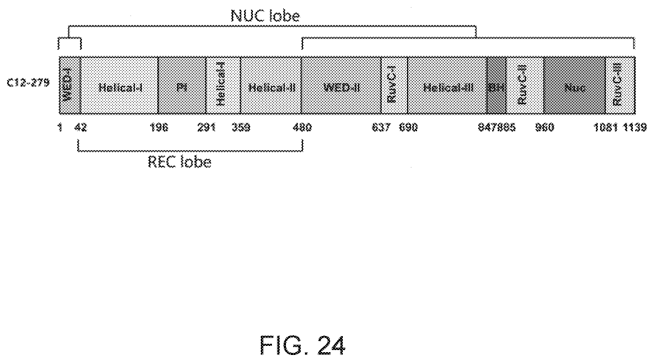

In the evolutionary tree, some proteins of the present disclosure form a separate and distinctly separated branch (a different cluster [CLUSTER]) compared to the known Cas12 isoform proteins, i.e., the proteins of the present disclosure are not mixed with the known Cas12 isoform proteins; and value of the alignment between these proteins in the present disclosure (FIG. 1B) and the existing Cas12 HMM Profile model is greater than 1e-5. These proteins of the present disclosure comprise CLUSTER1-CLUSTER13 proteins shown in FIG. 1B. Overall, this suggests that these Cas proteins may be novel subgroups, e.g., new Cas12 isoform proteins;

FIG. 2 is an SDS-PAGE electrophoresis pattern of a C12-279 recombinant protein according to some embodiment of the present disclosure;

FIG. 3 shows a PAM library for in vitro cleavage by Cas12 protein according to some embodiments of the present disclosure, and sequences in FIG. 3 are shown in SEQ ID NO: 878-881;

FIG. 4 shows a motif identified after C12-279-sgRNA targeting a 7nt random sequence according to some embodiments of the present disclosure;

FIG. 5 shows a motif identified after C12-279-sgRNA-Rev targeting a 7nt random sequence according to some embodiments of the present disclosure;

FIG. 6 shows fragments of plasmids containing 7nt random sequences in a plasmid elimination assay according to some embodiments of the present disclosure, and the sequences in FIG. 6 are shown in SEQ ID NO: 720 and SEQ ID NO: 882-886;

FIG. 7 shows a motif identified in the plasmid elimination assay for C12-279 according to some embodiments of the present disclosure;

FIG. 8 shows a partial indel result generated after C12-279 editing TTR genes according to some embodiments of the present disclosure, and the sequences in the FIG. 8 are shown in SEQ ID NO: 722 and SEQ ID NO: 887-916;

FIG. 9 shows that a CI1062732 protein (SEQ ID NO: 46) with dozens of amino acid residues deletion at an N-terminal of the C12-279 protein (SEQ ID NO: 696), in combination with a gRNA containing the DR sequence (GTAATGCGTCTCCCATTGACGCC) (SEQ ID NO: 529), targets a 7nt random sequence plasmid library in bacteria, grabbing a “false” PAM motif of 5′-TTNC-3.

FIG. 10 is a diagram of a secondary structure of a “false” DR sequence according to some embodiments of the present disclosure, hypothesizing that the 3′-end base C of the identified “false” PAM motif TTNC is caused by an extra C at the 3′ end of the DR, and the sequences in FIG. 10 are shown in SEQ ID NO: 917 and SEQ ID NO: 918.

FIG. 11 is an SDS-PAGE electrophoresis pattern of a C12-101-07 recombinant protein (126 KDa) according to some embodiments of the present disclosure;

FIG. 12 shows a motif identified after C12-101-07-sgRNA targeting a 7nt random sequence according to some embodiments of the present disclosure;

FIG. 13 shows a motif identified after C12-101-07-sgRNA-Rev targeting a 7nt random sequence according to some embodiments of the present disclosure;

FIG. 14 shows a motif identified in a plasmid elimination assay for C12-101-09 according to some embodiments of the present disclosure;

FIG. 15A shows fragments of pCDH-CMV-EGFP-reporter3-EF1-Puro plasmid according to some embodiments of the present disclosure; the sequences in FIG. 15A are shown in SEQ ID NO: 919-921; FIG. 15B shows fragments of a plasmid library with a PAM sequence of NAAN according to some embodiments of the present disclosure; and the sequences in FIG. 15B are shown in SEQ ID NO: 922-924;

FIG. 16 shows the editing efficiency of different C12-279 mutants in NAAN cells, expressed as multiples of the editing efficiency of C12-279, according to some embodiments of the present disclosure;

FIG. 17 shows the editing efficiency of different C12-279 mutants in NAAN cells, expressed as multiples of the editing efficiency of C12-279, according to some embodiments of the present disclosure;

FIG. 18 shows editing efficiency test results of different mutants targeting a reporter system according to some embodiments of the present disclosure, where Wt denotes a wild type C12-279; the marked number n indicates that the amino acid residue at position n is mutated to arginine R; Mut-01 is a mutant C12-279-pCDH-05 (Q186R mutant), Mut-02 is a mutant C12-279-pCDH-28 (double point mutation of Q186R and D352R), and Mut-03 is a mutant C12-279-pCDH-35 (triple point mutation of G184R, Q186R, and D352R); a lower dashed line indicates a 50% increase in editing efficiency compared to the wild type C12-279; an upper dashed line indicates an editing efficiency equal to the editing efficiency of Mut-02;

FIG. 19 shows editing efficiency test results of different multipoint mutants in the reporter system according to some embodiments of the present disclosure; where Mut-02-1-5-426-858-860 denotes a multipoint mutant obtained by additionally introducing mutations at positions 1, 5, 426, 858, and 860 (all mutate to arginine R) based on the Mut-02 mutant, i.e., the multipoint mutant contains the following mutations: amino acid residues at positions 1, 5, 186, 352, 426, 858, and 860 are all mutated to R; and other mutants are similar;

FIG. 20A shows an efficiency of multipoint mutants combined with different gRNAs in editing TTR gene according to some embodiments of the present disclosure; FIG. 20B shows an efficiency of multipoint mutants combined with different gRNAs in editing HBG gene according to some embodiments of the present disclosure; where Mut-02-1-5-426-858-860 denotes a multipoint mutant obtained by additionally introducing mutations at positions 1, 5, 426, 858, and 860 (all mutated to arginine R) based on the Mut-02 mutant, i.e., the multipoint mutant contains the following mutations: all amino acid residues at positions 1, 5, 186, 352, 426, 858, and 860 are mutated to R; and other mutants are similar;

FIG. 21 shows editing activity test results of dCas 12-279 according to some embodiments of the present disclosure; where Mut-02-426-860 denotes a multipoint mutant obtained by additionally introducing 426R and 860R mutations based on the Mut-02 mutant; Mut-02-426-860-D651A denotes a multipoint mutant obtained by additionally introducing 426R, 860R, and 651A mutations based on the Mut-02 mutant; and other mutants are similar;

FIG. 22 shows NGS sequencing results after the coding mRNA of the mutant Mut-02-1-5-426-858-860 is combined with the modified gRNA (C279-dmTTR01-02) for electroporation of HEK293 cells and editing of TTR gene, with an editing efficiency up to 92.18%, according to some embodiments of the present disclosure; and the sequences in FIG. 22 are shown as SEQ ID NO: 925-974;

FIG. 23 shows a PAM recognized by C12-279 mutant Mut-02-1-426-846-858-860 according to some embodiments of the present disclosure; and

FIG. 24 is a schematic diagram illustrating a structure of C12-279 according to some embodiments of the present disclosure.

### DETAILED DESCRIPTION

In the present disclosure, scientific and technical terms used herein have the meanings commonly understood by those of skill in the art unless otherwise indicated. Additionally, the procedures involving molecular genetics, nucleic acid chemistry, chemistry, molecular biology, biochemistry, cell culture, microbiology, cell biology, genomics, and recombinant DNA, as used herein, are all standard techniques widely employed in their respective fields. At the same time, for better understanding of the present disclosure, definitions and explanations of relevant terms are provided below.

In the present disclosure, the term “several” refers to a quantity greater than or equal to 2. In the present disclosure, the term “multiple” refers to a quantity greater than or equal to 2.

In the present disclosure, depending on the context, the term “cleavage” refers to cutting of a main chain of a polynucleotide chain; and non-limiting examples include complete cleavage of a single-stranded DNA, cleavage of one strand of a double-stranded DNA, or cleavage of both strands of a double-stranded DNA.

In the present disclosure, depending on the context, the term “modification” refers to other forms of chemical reactions of nucleic acid strands other than “cleavage”. It includes, but is not limited to, base substitution, addition, and/or deletion, as well as methylation and demethylation of nucleic acid strands. Non-limiting examples include base substitution on a target nucleic acid strand through single-base editing (e.g., the Cas12 of the present disclosure is fused with a deaminase domain and combined with gRNA), such as A→G, C→T, T→C, or G→A nucleotide mutations, as well as other types of nucleotide mutations (e.g., A→T, C→G, T→A, G→C, etc.). Other examples include base substitution, addition, or deletion through Prime editing technology (e.g., the Cas12 of the present disclosure is fused with a reverse transcriptase and combined with pegRNA), or base substitution, addition, or deletion through homology-directed repair (HDR) (e.g., the Cas12 of the present disclosure is combined with gRNA and a donor template). In addition, the Cas12 of the present disclosure is also fused with a DNA methyltransferase or a DNA demethylase and combined with gRNA for targeted modification.

In the present disclosure, depending on the context, the term “modulating an expression of a target nucleic acid” refers to modulation of the transcription of the target nucleic acid. Non-limiting examples include enhancing or suppressing the transcription of the target nucleic acid using CRISPRa or CRISPRi technologies, by means of a transcriptional activation or repression domain fused to Cas12.

In the present disclosure, letters in amino acid sequences denote single-letter abbreviations for amino acids well known in the art, as described in J. Biol. Chem, 243, p 3558 (1968), Alanine: Ala-A, Arginine: Arg-R, Aspartic acid: Asp-D, Cysteine: Cys-C, Glutamine: Gln-Q, Glutamic acid: Glu-E, Histidine: His-H, Glycine: Gly-G, Asparagine: Asn-N, Tyrosine: Tyr-Y, Proline: Pro-P, Serine: Ser-S, Methionine: Met-M, Lysine: Lys-K, Valine: Val-V, Isoleucine: Ile-I, Phenylalanine: Phe-F, Leucine: Leu-L, Tryptophan: Trp-W, and Threonine: Thr-T.

In the present disclosure, the term “amino acid difference” refers to the difference of amino acid residues at specific positions in the protein's amino acid sequence, including substitution, addition, or deletion.

In the present disclosure, a mutation of an amino acid residue at a specific position refers to the substitution, addition, or deletion of the amino acid residue at that position.

It is well known to those skilled in the art that in proteins or peptides, two adjacent amino acids each lose an OH or H through dehydration condensation to form a peptide bond, and each amino acid exists in the form of an amino acid residue. Thus, in the present disclosure, the terms “amino acid” and “amino acid residue” refer to the same meaning. Further, in the present disclosure, to simplify the expression, the amino acid residue before the substitution is retained before the position of the amino acid residue; the letter before the position indicates the original amino acid residue, and the letter after the position indicates the substituted amino acid residue. For example, “S211” represents that the original amino acid residue at position 211 is S, and when it is substituted with R, it may be expressed as “S211R”.

In the present disclosure, the symbol “+” is sometimes used to connect one amino acid mutation on each side, indicating that both point mutations are present simultaneously in a single mutant; if two or more point mutations are connected by two or more “+”, it represents that these point mutations are present simultaneously.

In the present disclosure, if an amino acid is substituted, it refers to that it is substituted with another amino acid residue different from the original amino acid residue. If the original amino acid is a positively charged amino acid and is substituted with a positively charged amino acid, it refers to that it is substituted with another positively charged amino acid residue different from the original one. For example, if an original amino acid residue is R and is substituted with a positively charged amino acid, it refers to that it is substituted with H or K.

In the present disclosure, when referring to an “RNA sequence”, “T” in the sequence is used interchangeably with “U”. When referring to a “guide sequence”, “T” in the sequence is used interchangeably with “U”. When referring to a “direct repeat (DR) sequence”, “T” in the sequence is used interchangeably with “U”.

In the present disclosure, when referring to the numbering of a Cas protein, C12-n and Cas12-n refer to the same protein. For example, Cas12-279 and C12-279 are used interchangeably.

### Sequence Identity

As used herein, the term “identity” refers to a sequence matching degree between two polypeptides or between two nucleic acids. The terms “identity”, “percent identity”, and “sequence identity” are used interchangeably. When a given position in two compared sequences are occupied by the same base or amino acid monomeric subunit (for example, if the same position in each of two DNA molecules is occupied by adenine, or the same position in each of two polypeptides is occupied by lysine), the molecules are considered to be identical at that position. The percent identity between two sequences is calculated by a function: the number of matching positions shared by the two sequences/the total number of compared positions×100%. For example, if there are 6 matching positions in 10 positions of two sequences, then the two sequences have 60% sequence identity. Typically, the alignment is performed by aligning the two sequences to generate the maximum sequence identity. Such alignment may be performed by using published and commercially available alignment algorithms and programs, including but not limited to CLUSTER Ω, MAFFT, Probcons, T-Coffee, Probalign, and BLAST, which may be reasonably selected and used by one of ordinary skill in the art. Those skilled in the art can determine appropriate parameters for sequence alignment, including any algorithm required to achieve an optimal or best alignment for the full length of the compared sequences, as well as any algorithm required to achieve an optimal or best alignment for the local region of the compared sequences.

### CRISPR-Cas12 System

As used herein, the terms “clustered regularly interspaced short palindromic repeats (CRISPR)-CRISPR-Cas system” or “CRISPR system” are used interchangeably and have the meanings commonly understood by those of skill in the art, which generally comprise transcription products or other elements associated with the expression of a CRISPR-associated (Cas) gene or transcription products or other elements capable of guiding the activity of the Cas gene. Such transcription products or other elements may comprise sequences encoding Cas effector proteins and guide polynucleotides.

Zhang Feng et al. discovered Cas12a in 2015, categorized as type V in the Class II CRISPR-Cas system. After detailed studies of subtype V-A (Cas12a), Zhang Feng et al. reported Cas12b (C2C1) in 2015. In 2017, Burstein et al. reported the Cas12e (CasX) nuclease. In 2019, Winston X. Yan et al. reported the newly discovered type V Cas effector proteins Cas12c, Cas12h, Cas12i, and Cas12g in detail by bioinformatics analysis.

In some embodiments, a Cas12 protein as described herein refers to a protein having an amino acid sequence, the amino acid sequence having at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to any one of sequences shown in SEQ ID NO: 1-53, SEQ ID NO: 696, or SEQ ID NO: 728. When the CRISPR-Cas12 system comprises a fusion protein or a conjugate comprising the Cas12 protein and a functional domain, a percent sequence identity between the Cas12 portion of the fusion protein or the conjugate and a reference sequence is calculated.

In the present disclosure, the CRISPR-Cas12 system comprises the Cas12 protein with the amino acid sequence having at least 50% sequence identity to any one of sequences shown in SEQ ID NO: 1-53, SEQ ID NO: 696, and SEQ ID NO: 728, or a nucleic acid encoding the Cas12 protein; and a guide polynucleotide or a nucleic acid encoding the guide polynucleotide; the guide polynucleotide comprises a DR sequence linked to a guide sequence, the guide sequence is engineered to hybridize with a target nucleic acid, and the guide polynucleotide is capable of forming a complex with the Cas12 protein and guiding the sequence-specific binding of the complex to the target nucleic acid.

### Guide Polynucleotide

As used herein, the term “guide polynucleotide” refers to a molecule in a CRISPR-Cas system that forms a complex with the Cas protein and guides the complex to a target sequence. Typically, the guide polynucleotide comprises a scaffold sequence that is linked to a guide sequence, and the guide sequence may hybridize to the target sequence. Typically, the scaffold sequence comprises a DR sequence, and sometimes, the scaffold sequence comprises a tracrRNA sequence. In some embodiments, the guide polynucleotide does not comprise a tracrRNA sequence. In some embodiments, the guide polynucleotide comprises a tracrRNA sequence.

In some embodiments, the guide polynucleotide of the CRISPR-Cas12 system is a guide RNA. In some embodiments, the guide polynucleotide is a chemically modified guide polynucleotide. In some embodiments, the guide polynucleotide comprises at least one chemically modified nucleotide.

In some embodiments, the chemically modified nucleotide comprises a base-modified nucleotide, a phosphate-modified nucleotide, and a ribose-modified nucleotide.

In some embodiments, the base-modified nucleotide is selected from nucleotides containing non-natural bases.

In some embodiments, the phosphate-modified nucleotide is selected from an aminophosphate nucleotide, a phosphorothioate nucleotide, a dithiophosphate nucleotide, a methylphosphonate nucleotide, a 5′-phosphate nucleotide, an alkyl phosphate nucleotide, and a borane phosphate nucleotide.

In some embodiments, the ribose-modified nucleotide is selected from a deoxynucleotide, a 3′-terminal deoxythymidine (dT) nucleotide, a 2′-O-methyl-modified nucleotide, a 2′-fluoro-modified nucleotide, a 2′-deoxy-modified nucleotide, a 2′-amino-modified nucleotide, a 2′-O-allyl-modified nucleotide, a 2′-C-alkyl-modified nucleotide, a 2′-hydroxy-modified nucleotide, a 2′-methoxyethyl-modified nucleotide, a 2′-O-alkyl-modified nucleotide, and a morpholino nucleotide.

In some embodiments, the base-modified nucleotide is selected from nucleotides containing non-natural bases, for example, 5-methylcytosine, 5-hydroxymethylcytosine, pseudouridine, 2,6-diaminopurine, 2-thiopurine, 7-methylguanosine, 8-bromoguanosine, 5-iodouracil, 5-bromouracil, 5-propargyluracil, 5-methyluracil, 1-methylpseudouridine, N6-methyladenosine, N6-methylthioadenosine, 2-aminopurine, isocytosine, isoguanine, and isoguanine. In some embodiments, the phosphate-modified nucleotide is selected from an aminophosphate nucleotide, a phosphorothioate nucleotide, a phosphorodithioate nucleotide, a methylphosphonate nucleotide, a 5′-phosphorylated nucleotide, an alkylphosphate nucleotide, a boranephosphonate nucleotide, a phosphoroselenoate nucleotide, a fluorophosphonate nucleotide, an allylphosphate nucleotide, a benzylphosphate nucleotide, a cyanophosphonate nucleotide, and a sulfonate-modified nucleotide. In some embodiments, the ribose-modified nucleotide is selected from deoxynucleotide, 3′-deoxythymidine nucleotide, 2′-O-methyl nucleotide, 2′-fluoro nucleotide, 2′-deoxy nucleotide, 2′-amino nucleotide, 2′-O-allyl nucleotide, 2′-C-alkyl nucleotide, 2′-hydroxy nucleotide, 2′-O-methoxyethyl (MOE) nucleotide, 2′-O-alkyl nucleotide, morpholino nucleotide (PMO), locked nucleic acid (LNA), thio-sugar nucleotide, 2′-O-methoxy nucleotide, 4′-methyl nucleotide, 3′-O-alkyl nucleotide, 3′-amino nucleotide, 4′-thionucleotide, cyclo-nucleotide, peptide nucleic acid (PNA), β-deoxyinosine nucleotide, 2′-fluoro-deoxynucleotide, 2′-protected nucleotide (for stabilizing CRISPR RNP), methylated ribose-modified nucleotide, and hydrophobic-tailed nucleotide (for cell membrane penetration).

In some embodiments, the guide polynucleotide comprises a chemically modified nucleotide at a 5′ end and/or a 3′ end.

In some embodiments, the guide polynucleotide comprises a deoxynucleotide at the 5′ end; and the deoxynucleotide has a length in a range of 10 to 25 nt, e.g., 14 nt or 25 nt.

In some embodiments, the guide polynucleotide comprises a nucleotide with phosphorothioate group and 2′-O-methyl modifications at the 5′ end or the 3′ end.

In some embodiments, the guide polynucleotide comprises at least one guide sequence (or referred to as a spacer sequence) linked to at least one DR sequence. In some embodiments, the guide sequence is located at the 3′ end of the DR sequence. In some embodiments, the guide sequence is located at the 5′ end of the DR sequence.

In some embodiments, the tracrRNA sequence is linked to the DR sequence.

In some embodiments, the tracrRNA sequence is located at the 5′ end or 3′ end of the DR sequence. In some embodiments, the tracrRNA sequence is located at the 5′ end of the DR sequence. In some embodiments, the tracrRNA sequence is located at the 3′ end of the DR sequence.

In some embodiments, a nucleotide sequence of the guide polynucleotide comprises the tracrRNA, the DR sequence, and the guide sequence in order from the 5′ end to the 3′ end.

In some embodiments, the nucleotide sequence of the guide polynucleotide comprises the tracrRNA, a linker sequence, the DR sequence, and the guide sequence in order from the 5′ end to the 3′ end.

In some embodiments, the nucleotide sequence of the guide polynucleotide comprises the tracrRNA, a loop sequence, the DR sequence, and the guide sequence in order from the 5′ end to the 3′ end.

In some embodiments, a structure of the guide polynucleotide is as follows: 5′-tracrRNA-loop sequence-DR sequence-guide sequence-3′.

In some embodiments, the tracrRNA and the DR sequence of the guide polynucleotide are linked by a nucleotide sequence.

In some embodiments, the tracrRNA sequence is linked to the DR sequence by a nucleotide sequence including 1, 2, 3, 4, 5, 6, 7, 8, 9, or 10 nucleotides. In some embodiments, the tracrRNA sequence is linked to the DR sequence by a nucleotide sequence including 4 nucleotides. In some embodiments, the tracrRNA sequence is linked to the DR sequence by a 5′-GAAA-3′ sequence.

In some embodiments, the guide sequence is sufficiently complementary to a target nucleic acid to hybridize with the target nucleic acid and to guide sequence-specific binding of a CRISPR-Cas12 complex to the target nucleic acid. In some embodiments, the guide sequence has 100% complementarity with the target nucleic acid, but the guide sequence may also have less than 100% complementarity with the target nucleic acid, for example, at least 80%, at least 85%, at least 90%, at least 95%, at least 98%, or at least 99% complementarity.

In some embodiments, the guide sequence is engineered to hybridize to the target nucleic acid and is mismatched to the target nucleic acid by no more than two nucleotides. In some embodiments, the guide sequence is engineered to hybridize to the target nucleic acid and is mismatched to the target nucleic acid by no more than one nucleotide. In some embodiments, the guide sequence is engineered to hybridize to the target nucleic acid and is not mismatched or is mismatched to the target nucleic acid.

In some embodiments of the present disclosure, the guide sequence has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to any one of sequences shown in SEQ ID NO: 722, SEQ ID NO: 761-782, and SEQ ID NO: 825-877. In some embodiments of the present disclosure, the guide sequence is shown in any one of SEQ ID NO: 722, SEQ ID NO: 761-782, and SEQ ID NO: 825-877.

In some embodiments, the CRISPR-Cas12 system comprises at least 2, at least 3, at least 4, at least 5, at least 10, or at least 20 different guide polynucleotides. In some embodiments, the guide polynucleotide targets at least 2, at least 3, at least 4, at least 5, at least 10, or at least 20 different target nucleic acid molecules, or targets at least 2, at least 3, at least 4, at least 5, at least 10, or at least 20 different regions of one or more target nucleic acid molecules.

In some embodiments, the guide polynucleotide comprises a constant DR sequence located upstream of a variable guide sequence. In some embodiments, a plurality of guide polynucleotides is a portion of an array, which may be a portion of a vector, such as a viral vector or plasmid. For example, a guide array that comprises a sequence: DR sequence-spacer-DR sequence-spacer-DR sequence-spacer- . . . -DR sequence-spacer may comprise a plurality of unique unprocessed guide polynucleotides (one for each DR sequence-spacer or space-DR sequence). Once introduced into a cell or cell-free system, the array is processed by the Cas12 protein into several individual mature guide polynucleotides. This allows for multiplexing, such as delivering a plurality of guide polynucleotides into the cell or system to target a plurality of target nucleic acids or a plurality of regions within a single target nucleic acid.

The ability of the guide polynucleotide to guide the sequence-specific binding of the complex (a CRISPR complex) to the target nucleic acid may be assessed by any suitable assay. For example, components of the CRISPR system sufficient to form the complex (the CRISPR complex), including a guide polynucleotide to be tested, may be delivered to a host cell containing the corresponding target nucleic acid molecules, such as by transfection with a vector encoding the components of the CRISPR complex, followed by assessment of preferential cleavage within a target sequence. Similarly, cleavage of the target nucleic acid sequence may be assessed in vitro by providing the target nucleic acid and the components of the CRISPR complex including the guide polynucleotide to be tested and a control guide polynucleotide different from the guide polynucleotide to be tested, and then comparing the ability of the guide polynucleotide to be tested and the control guide polynucleotide to bind the target nucleic acid or the rate of the guide polynucleotide to be tested and the control guide polynucleotide to cleave the target nucleic acid. The ability of the CRISPR complex to cleave or bind the target nucleic acid may also be assessed by the manner described above.

### Cas12 Mutant

As described herein, when referring to “a position corresponding to a sequence shown in SEQ ID NO: XX” or a similar textual description, the position may be determined by amino acid sequence alignment. Typically, the alignment is made when two sequences are aligned to produce a maximum sequence identity. Such an alignment may be performed by using published and commercially available alignment algorithms and programs such as, but not limited to, Clustal 22, MAFFT, Probcons, T-Coffee, Probalign, and BLAST, which may be reasonably selected by one of ordinary skill in the art. One skilled in the art can determine appropriate parameters for sequence alignment, including any algorithm needed to achieve an optimal or best alignment for the full length of the compared sequences, as well as any algorithm required to achieve an optimal or best local alignment for the local region of the compared sequences.

In some embodiments, the corresponding position is determined by performing an online sequence alignment of an amino acid sequence of the Cas12 protein with any one of sequences shown in SEQ ID NO: 1-53, SEQ ID NO: 696, and SEQ ID NO: 728 using the MAFFT version 7 tool (mafft.cbrc.jp), with the following parameters: G-INS-i (Very slow; recommended for <200 sequences with global homology; 2 iterative cycles only), Try to align gappy regions anyway, Scoring matrix for amino acid sequences-BLOSUM62, Gap opening penalty 1.53, Offset value 0.0, and Mafft-homologs-Use UniRef50 (more comprehensive and requires longer search time).

In some embodiments, the corresponding position is determined by performing an online sequence alignment of the amino acid sequence of the Cas12 protein with the sequence shown in SEQ ID NO: 696 using the MAFFT version 7 tool (mafft.cbrc.jp), with the following parameters: G-INS-i (very slow; recommended for <200 sequences with global homology; 2 iterative cycles only), Try to align gappy regions anyway, Scoring matrix for amino acid sequences-BLOSUM62, Gap opening penalty 1.53, Offset value 0.0, Mafft-homologs-Use UniRef50 (more comprehensive and requires longer search time).

In some embodiments, the Cas12 protein herein comprises one or more mutations, e.g., a single amino acid insertion, a single amino acid deletion, a single amino acid substitution, or any combination thereof compared to the Cas12 protein with a sequence shown in any one of SEQ ID NO: 1-53, SEQ ID NO: 696, and SEQ ID NO: 728. In some embodiments, compared to the Cas12 protein with the sequence shown in any one of SEQ ID NO: 1-53, SEQ ID NO: 696, and SEQ ID NO: 728, the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 amino acid changes (e.g., insertions, deletions, or substitutions), but retains the ability to bind to a target nucleic acid molecule that is complementary to a guide sequence of a guide polynucleotide, and/or the ability to process an RNA transcript containing a guide sequence into a guide polynucleotide molecule. In some embodiments, compared to the Cas12 protein with the sequence shown in any one of SEQ ID NO: 1-53, SEQ ID NO: 696, and SEQ ID NO: 728, the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 amino acid changes (e.g., insertions, deletions, or substitutions), but retains the ability to bind a target nucleic acid molecule that is complementary to the guide sequence of the guide polynucleotide.

One type of modification or mutation comprises replacing an amino acid residue with an amino acid having similar biochemical properties, i.e., a conservative substitution. Usually, the conservative substitution has little or no effect on the activity of the resulting protein or peptide. For example, the conservation substitution refers to a substitution of an amino acid residue in the Cas12 protein that does not substantially affect the binding between the Cas12 protein and a target nucleic acid molecule that is complementary to a guide sequence of a gRNA molecule, and/or the process of processing a guide array RNA transcript into gRNA molecules.

More substantial changes may be introduced by using fewer conservative substitutions, e.g., by selecting residues that differ more significantly in maintaining the following effects: (a) the polypeptide backbone structure in the region where the substitution occurs, such as a helical or folded conformation; (b) the charge or hydrophobicity of the region interacted with the target site; or (c) the bulk of the amino acid side chain. The substitutions that are generally expected to produce the greatest changes in peptide function are (a): a substitution between hydrophilic residues (e.g., serine or threonine) and hydrophobic residues (e.g., leucine, isoleucine, phenylalanine, valine, or alanine); (b) a substitution between cysteine or proline and any other residue; (c) a substitution between residues with a positively charged side chain (e.g., lysine, arginine, or histidine) and residues with a negatively charged residue (e.g., glutamic acid or aspartic acid); or (d) a substitution between a residue having a bulky side chain (e.g., phenylalanine) and a residue not having a side chain (e.g., glycine).

### Cas12 Active Fragment

In the present disclosure, the Cas12 protein may comprise only a WED-I domain, a Helical-I1 domain, a PI domain, a Helical-I2 domain, a Helical-II domain, a WED-II domain, a Ruvc-I domain, a Helical-III domain, a BH domain, a Ruvc-II domain, a Nuc domain, and/or a Ruvc-III domain.

The Cas12 protein described herein, in addition to including the domains described above, may also comprise domains of the Cas12 proteins in the prior art, which together form a complete structure of the Cas12 protein to fulfill the function of the Cas12 protein described in the present disclosure. The function comprises, but is not limited to, retaining the ability of the Cas12 protein to form a complex with a gRNA, retaining the ability of the Cas12 protein to form a complex with a gRNA and target a target nucleic acid, retaining the ability of the complex formed by the Cas12 protein with the gRNA to target and modulate the expression of the target nucleic acid, retaining the ability of the complex formed by the Cas12 protein with the gRNA to target and cleave a single strand or double strands of a target nucleic acid, retaining the ability of the Cas12 protein to bind a target nucleic acid molecule that is complementary to a guide sequence of a guide polynucleotide, and/or retaining the ability to process RNA transcripts containing the guide sequence into guide polynucleotide molecules.

For example, in some embodiments, the Cas12 active fragment comprises only the PAM-interacting (PI) domain of C12-279 or a homologous sequence thereof. Such active fragment is capable of recognizing a PAM sequence on the target nucleic acid. Furthermore, the active fragment may be used to replace a PI domain in another Cas12 protein (e.g., a Cas12i protein, which is known in the literature to typically recognize a TTN PAM), such that the newly formed chimeric protein can recognize a WTN or ATN PAM.

In some other embodiments, the Cas12 active fragment comprises only the REC lobe of C12-279 or a homologous sequence thereof. Such active fragment may retain the ability to bind a guide polynucleotide and/or a target nucleic acid.

In some other embodiments, the Cas12 active fragment comprises only the RuvC nuclease domain(s) of C12-279 or homologous sequences thereof. Such a fragment may retain the ability to cleave a target nucleic acid when provided in combination with a guide polynucleotide. For example, the RuvC domain may be fused to heterologous DNA-binding domains, or other CRISPR effector proteins to generate engineered nucleases with altered specificity or activity.

### Inactivated Cas12 Mutant

By inactivating the RuvC domain of Cas12 through introducing point mutations, the Cas12 protein loses its endonuclease activity, resulting in a dCas12 that can only bind to a target gene under the mediation of the guide polynucleotide but does not possess the function of cleaving DNA.

Point mutations may also be introduced to partially inactivate the RuvC domain of Cas12, resulting in a nickase Cas12 (nCas12), which can bind to a target gene and cleave only one strand of the double-stranded nucleic acid under the mediation of the guide polynucleotide, while leaving the other strand intact.

Accordingly, the dCas12 or the nCas12 may be fused or conjugated with other domains (including, but not limited to, deaminase domains, transcriptional activation domains, transcriptional repression domains, methylation domains, demethylation domains, histone acetylation domains, and histone deacetylation domains), and guided to a target sequence of a target nucleic acid by the guide polynucleotide, to exert corresponding functions through the other domains. For example, the conversion of cytosine (C) to thymine (T) in the target nucleic acid is achieved by deamination of the cytosine base; the conversion of adenine (A) to guanine (G) is achieved by deamination of the adenine base; the transcriptional repression of the target nucleic acid is achieved using the transcriptional repression domain KRAB; and the transcriptional activation is promoted using the transcriptional activation domain VP64.

### Functional Domain

In some embodiments, the Cas12 protein or the inactivated Cas12 mutant is covalently linked or fused to a homologous or heterologous functional domain.

In some embodiments, the functional domain has an enzyme activity that modifies a target nucleic acid sequence; the enzyme activity comprising a nuclease activity, a methyltransferase activity, a demethylase activity, a DNA repair activity, a DNA damage activity, a deamination activity, a dismutase activity, an alkylation activity, a depurination activity, an oxidation activity, a pyrimidine dimer formation activity, an integrase activity, a transposase activity, a recombinase activity, a polymerase activity, a ligase activity, a helicase activity, a photolyase activity, a glycosylase activity, a deglycosylation activity, an acetyltransferase activity, a deacetylase activity, a kinase activity, a phosphatase activity, a ubiquitin ligase activity, a deubiquitination activity, an adenylylation activity, a deadenylation activity, a SUMOylating activity, a deSUMOylating activity, a myristoylation activity, and/or a demyristoylation activity.

In some embodiments, the functional domain is selected from one or more of the following: a nuclease (e.g., FokI), a methyltransferase, a demethylase, a DNA repair enzyme, a DNA damage enzyme, a deaminase, a dismutase, an alkylase, a depurinase, an oxidase, a pyrimidine dimer-forming enzyme, an integrase, a transposase, a recombinase, a polymerase, a ligase, a helicase, a photolyase, a glycosylase, a deglycosylase, an acetyltransferase, a deacetylase, a kinase, a phosphatase, a ubiquitin ligase, a deubiquitinating enzyme, an adenylylase, a deadenylase, a SUMOylating enzyme, a deSUMOylating enzyme, a myristoylase, and/or a demyristoylase.

In some embodiments, the functional domain is selected from one, two, three, four, or more of the following: a subcellular positioning signal, a DNA binding domain, a protease domain, a transcriptional activation domain, a transcriptional repression domain, a nuclease domain, a deaminase domain, a uracil DNA glycosylase domain (UDG), a uracil DNA glycosylase inhibitory domain (UGI), a methylase, a demethylase, a transcription release factor, a histone acetylase domain, a histone deacetylase domain, a DNA ligase, an affinity tag, a reporter tag, an affinity domain, and a reporter domain.

In some embodiments of the present disclosure, the deaminase domain is selected from the following: APOBEC1, APOBEC3A, APOBEC3B, APOBEC3C, APOBEC3D, APOBEC3F, an activation-induced cytidine deaminase (AID), cytidine deaminase (CDA) from lamprey, and engineered mutants of adenosine deaminase (TadA) that act on DNA.

In some embodiments, the transcriptional activation domain is selected from the following: P65, VPR, VP16, VP64, VTR1, VTR2, VTR3, p65, MyoD1, HSF1, RTA, SET7/9, and a histone acetyltransferase. In some embodiments, the transcriptional activation domain is selected from the following: the sequence ETFSDLWKL from p53 TAD1, the sequence DDIEQWFTE from p53 TAD2, the sequence SDIMDFVLK from MLL, the sequence DLLDFSMMF from E2A, the sequence ETLDFSLVT from Rtg3, the sequence RKILNDLSS from CREB, the sequence EAILAELKK from CREBaB6, the sequence DDVVQYLNS from Gli3, the sequence DDVYNYLFD from Gal4, the sequence DLFDYDFLV from Oaf1, the sequence DFFDYDLLF from Pip2, the sequence EDLYSILWS from Pdr1, and the sequence TDLYHTLWN from Pdr3.

In some embodiments, the transcriptional repression domain is selected from: KRAB domain of KOX1, KRAB domain of KAP-1, MAD, FKHR, EGR-1, ERD, SID, a tandem repeat of SID (e.g., SID4X), KRAB domain of TIEG, v-ERB-A, MBD2, MBD3, TRa, a histone methyltransferase, a histone deacetylase (HDAC), a nuclear hormone receptor (e.g., an estrogen receptor or a thyroid hormone receptor), members of the DNMT family (e.g., DNMT1, DNMT3A, DNMT3B), the KRAB domain of MeCP2, ROM2, and AtHD2A.

In some embodiments, the transcriptional repression domain is a KRAB domain. In some embodiments, the transcriptional repression domain is a KRAB domain from a KOX1 protein.

In some embodiments, the nuclease domain is selected from the following: FokI, a polypeptide with single-stranded DNA (ssDNA) cleavage activity, or a polypeptide with double-stranded DNA (dsDNA) cleavage activity.

In some embodiments, the methylase domain is selected from a DNA methylase, including, but not limited to, DNMT1, DNMT3a, and DNMT3b.

In some embodiments, the demethylase is selected from TET1CD, TET1, ROS1, DME, DML2, and DML3.

Methylation and demethylation are recognized in the field as important modes of epigenetic gene modulation.

In some embodiments, the homologous or heterologous functional domain refers to a sequence tag useful for the solubility, purification, or detection of the fusion protein or conjugate. Suitable protein tag sequences are provided in the present disclosure, which include, but are not limited to, a biotin carboxylase carrier protein (BCCP) tag, a myc tag, a calmodulin tag, a FLAG tag, a hemagglutinin (HA) tag, a polyhistidine tag (also known as His tag), a maltose-binding protein (MBP) tag, a nus tag, and a glutathione-S-transferase (GST) tag, a green fluorescent protein (GFP) tag, a thioredoxin tag, a S-tag, a Softag (e.g., Softag 1, Softag 3), a strep-tag, a biotin ligase tag, a FLASH tag, a V5 tag, and a SBP tag. Additional suitable sequences are apparent to those of ordinary skill in the art.

In some embodiments of the present disclosure, a single-base editor is constructed by fusing a Mut-02-1-426-846-858-860-D651A-E891A-D1082A mutant with a deaminase domain and a nuclear localization signal (NLS). In some embodiments of the present disclosure, a single-base editor is constructed by fusing a Mut-02-1-426-846-858-860-D651A-E891A-D1082A mutant with an APOBEC3A domain and an SV40 NLS.

In some embodiments of the present disclosure, a transcriptional repression epigenetic editor is constructed by fusing a Mut-02-1-426-846-858-860-D651A-E891A-D1082A mutant with a KRAB domain and an SV40 NLS. In some embodiments of the present disclosure, a transcriptional activation epigenetic editor is constructed by fusing a Mut-02-1-426-846-858-860-D651A-E891A-D1082A mutant with a VP64 domain and an SV40 NLS.

### Subcellular Localization Signal

In some embodiments, the Cas12 protein is fused to at least one type of homologous or heterologous subcellular localization signal. In some embodiments, the Cas12 protein is fused to at least one homologous or heterologous subcellular localization signal. Exemplarily, the subcellular localization signal comprises an organelle localization signal, such as a nuclear localization signal (NLS), a nuclear export signal (NES), or a mitochondrial localization signal.

Non-limiting examples of NLS include NLS sequences derived from: an NLS of SV40 large T antigen having the amino acid sequence PKKKKRKV (SEQ ID NO: 738); an NLS of a nucleoplasmic protein (e.g., a sequence KRPAATKKKAGQAKKKKK, SEQ ID NO: 739); an NLS of c-myc having the amino acid sequence PAAKRVKLD (SEQ ID NO: 740) or the amino acid sequence RQRRNELKRSP (SEQ ID NO: 741); an NLS of hRNPA1 M9 having the amino acid sequence NQSSNFGPMKGGGNFGGRSSGPYGGGGGQYFAKPRNQGGY (SEQ ID NO: 742); a sequence RMRIZFKNKGKDTAELRRRRVEVSVELRKKAKKDEQILKRRNV (SEQ ID NO: 743) derived from an IBB domain; a sequence VSRKRPRP (SEQ ID NO: 744) and a sequence PPKKARED (SEQ ID NO: 745) of the rhabdomyosarcoma T-protein; a sequence PQPKKKPL of human p53 (SEQ ID NO: 746); a sequence SALIKKKKKKMAP (SEQ ID NO: 747) of mouse c-ablIV; a sequence DRLRR (SEQ ID NO: 748) and a sequence PKQKKRK (SEQ ID NO: 749) of influenza virus NS1; and a sequence RKLKKKKKKKL (SEQ ID NO: 750) of hepatitis virus delta antigen; a sequence REKKKKFLKRR (SEQ ID NO: 751) of mouse Mx1 protein; a sequence KRKGDEVDGVDEVAKKKSKK (SEQ ID NO: 752) of human poly(ADP-ribose) polymerase; and a sequence RKCLQAGMNLEARKTKKK (SEQ ID NO: 753) of steroid hormone receptor. In some embodiments, the nuclear localization sequence has sufficient strength to drive the accumulation of the fusion protein or conjugate described herein within the nucleus of a eukaryotic cell to a detectable level. In summary, the strength of the nuclear localization activity may be derived from a count of the NLS, one or more specific used NLSs, or any combination of these factors. The accumulation within the nucleus may be detected using any suitable technique. For example, a detectable marker may be fused to the Cas protein to allow visualization of its intracellular location, such as in combination with detection methods of nuclear location (e.g., nucleus-specific dyes such as DAPI)). As another example, the cell nucleus may be isolated from the cell, and its contents are subsequently analyzed using any appropriate method for detecting protein, including but not limited to immunohistochemistry, western blotting, or enzymatic activity assays. As another example, the accumulation within the nucleus may also be indirectly determined, for example, by assessing the effect of the formation of a nucleic acid-targeting complex (e.g., measuring DNA or RNA cleavage or mutation at a target sequence, or measuring changes in gene expression activity resulting from the formation of a DNA-targeting complex or a RNA-targeting complex and/or the activity of a DNA-targeting Cas protein or a RNA-targeting Cas protein), compared with a control group that is not exposed to a nucleic acid-targeting Cas protein or complex, or exposed to a nucleic acid-targeting Cas protein lacking one or more NLSs.

### Vector System

Some embodiments of the present disclosure relate to a vector system comprising the CRISPR-Cas12 system described herein. The vector system comprises one or more recombinant vectors, and the recombinant vector comprises a polynucleotide sequence encoding the Cas12 protein and a polynucleotide sequence encoding the guide polynucleotide.

In some embodiments, the vector system comprises at least one plasmid or viral recombinant vector (e.g., retrovirus, lentivirus, adenovirus, adeno-associated virus, or herpes simplex virus). In some embodiments, the polynucleotide sequence encoding the Cas12 protein and the polynucleotide sequence encoding the guide polynucleotide are located at the same recombinant vector. In some embodiments, the polynucleotide sequence encoding the Cas12 protein and the polynucleotide sequence encoding the guide polynucleotide are located at a plurality of recombinant vectors.

In some embodiments, the polynucleotide sequence encoding the Cas12 protein and/or the polynucleotide sequence encoding the guide polynucleotide is operably linked to a regulatory sequence (also known as a regulatory element). The regulatory element comprises a promoter, an enhancer, an internal ribosome entry site (IRES), and other expression control elements (e.g., a transcriptional termination signal such as a polyadenylation signal and a poly-U sequence). The regulatory element comprises an element that enables constitutive expression of the nucleotide sequence in many types of host cell types, as well as an element that restrict expression to specific host cells (e.g., a tissue-specific regulatory sequence). A tissue-specific promoter can be directly expressed primarily in the desired tissue of interest, e.g., muscle, neurons, bone, skin, blood, specific organs (e.g., liver, pancreas), or specific cell types (e.g., lymphocytes). The regulatory element may also guide expression in a time-dependent manner, e.g., in a cell-cycle-dependent or developmental-stage-dependent manner, which may or may not also be tissue-type specific or cell-type specific. In some embodiments, the regulatory element is enhancer elements, such as a WPRE, a CMV enhancer, an R-U5 segment in the LTR of HTLV-1, an SV40 enhancer, or an intronic sequence between exons 2 and 3 of the rabbit β-globin.

In some embodiments, the recombinant vector comprises a polymerase III (pol III) promoter (e.g., a U6 promoter and an H1 promoter), a polymerase II (pol II) promoter (e.g., the retroviral Rous sarcoma virus (RSV) long terminal repeat (LTR) promoter (optionally with an RSV enhancer), a cytomegalovirus (CMV) promoter (optionally with a CMV enhancer), an SV40 promoter, a dihydrofolate reductase promoter, a β-actin promoter, a phosphoglycerol kinase (PGK) promoter, or an EF1a promoter), or both a pol III promoter and a pol II promoter.

In some embodiments, the promoter is a constitutive promoter, which is continuously active and not modulated by external signals or molecules. Suitable constitutive promoters include, but are not limited to, CMV, RSV, SV40, EF1α, CAG, and β-actin promoters. In some embodiments, the promoter is an inducible promoter modulated by an external signal or molecule (e.g., a transcription factor).

In some embodiments, the promoter is a tissue-specific promoter, which may be used to drive tissue-specific expression of the Cas12 protein. Suitable muscle-specific promoters include, but are not limited to, CK8, MHCK7, a myoglobin (Mb) promoter, a desmin promoter, a muscle creatine kinase (MCK) promoter and mutants thereof, and an SPc5-12 synthesis promoter. Suitable immune cell-specific promoters include, but are not limited to, a B29 promoter (B cells), a CD14 promoter (monocytes), a CD43 promoter (leukocytes and platelets), a CD68 (macrophages) promoter, and an SV40/CD43 promoter (leukocytes and platelets). Suitable blood cell-specific promoters include, but are not limited to, a CD43 promoter (leukocytes and platelets), a CD45 promoter (hematopoietic cells), INF-β (hematopoietic cells), a WASP promoter (hematopoietic cells), an SV40/CD43 promoter (leukocytes and platelets), and an SV40/CD45 promoter (hematopoietic cells). Suitable pancreas-specific promoters include, but are not limited to, an elastase-1 promoter. Suitable endothelial cell-specific promoters include, but are not limited to, a Fit-1 promoter and an ICAM-2 promoter. Suitable neuronal tissue/cell-specific promoters include, but are not limited to, a GFAP promoter (astrocytes), an SYN1 promoter (neurons), and NSE/RU5′ (mature neurons). Suitable kidney-specific promoters include, but are not limited to, a NphsI promoter (podocytes). Suitable bone-specific promoters include, but are not limited to, an OG-2 promoter (osteoblasts, dentinogenic cells). Suitable lung-specific promoters include, but are not limited to, an SP-B promoter (lung). Suitable liver-specific promoters include, but are not limited to, an SV40/Alb promoter. Suitable heart-specific promoters include, but are not limited to, α-MHC. In some embodiments, the tissue-specific promoter is selected from liver-specific promoters such as an albumin (ALB) promoter, an alpha-fetoprotein (AFP) promoter, a transthyretin (TTR) promoter, a hepatocyte nuclear factor 4 alpha (HNF4a) promoter, an apolipoprotein B (APOB) promoter, a carbamoyl-phosphate synthase 1 (CPS1) promoter, and a coagulation factor VII promoter (F7 promoter); a hematopoietic stem/progenitor cell (HSC)-specific promoter such as a cluster of differentiation 34 (CD34) promoter, a stem cell leukemia (SCL) promoter, a KIT proto-oncogene (c-Kit) promoter, a GATA binding protein 2 (GATA2) promoter, a LIM domain only 2 (LM02) promoter, and a runt-related transcription factor 1 (RUNX1) promoter; a nerve-specific promoter such as a synapsin I promoter, a neuron-specific enolase (NES) promoter, a tyrosine hydroxylase (TH) promoter, a glial fibrillary acidic protein (GFAP) promoter, and a myelin basic protein (MBP) promoter; muscle-specific promoters such as a muscle creatine kinase (MCK) promoter, a desmin promoter, an alpha-myosin heavy chain (α-MHC) promoter, and a myogenin promoter; immune/hematopoietic lineage-specific promoters such as a cluster of differentiation 19 (CD19) promoter, a cluster of differentiation 3 epsilon (CD3ε) promoter, a CD8 promoter, a Integrin alpha M (CD11b) promoter, an interleukin-2 (IL-2) promoter, and a lymphocyte-specific protein tyrosine kinase (Lck) promoter; lung-specific promoters such as a surfactant protein C (SP-C) promoter, a clara cell 10-kDa protein (CC10) promoter, and a forkhead box protein J1 (FOXJ1) promoter; heart-specific promoters such as an alpha-myosin heavy chain (α-MHC) promoter, a cardiac troponin T (cTnT) promoter, a myosin light chain 2 ventricular (MLC-2v) promoter; and kidney-specific promoters such as a nephrin (NPHS1) promoter and an aquaporin 2 (AQP2) promoter.

In some embodiments, the tissue-specific promoter is selected from pancreatic/islet β-cell-specific promoters such as an insulin (INS) promoter, a pancreatic and duodenal homeobox 1 (PDX1) promoter, and a glucose transporter type 2 (GLUT2) promoter; and an intestinal-specific promoter such as a villin promoter, a mucin-2 (MUC2) promoter, and a fatty acid binding protein 2 (FABP2) promoter.

### Adeno-Associated Virus (AAV) Vector

Some embodiments of the present disclosure relate to an AAV vector comprising the CRISPR-Cas12 system, and the AAV vector comprises DNA encoding the Cas12 protein and the guide polynucleotide.

Delivery of the CRISPR-Cas system via the AAV vector was described in Maeder et al., Nature Medicine 25:229-233 (2019). It clinically demonstrated the safety and efficacy of subretinal delivery of AAV. Localized delivery via subretinal injection, the natural tropism of AAV5 for photoreceptor cells, and the use of the photoreceptor-specific GRK1 promoter were all employed to restrict expression of the CRISPR/Cas system to the therapeutic target tissue and cell types. The entire contents of this reference are incorporated herein by reference. In some embodiments, the AAV vector comprises a ssDNA genome that comprises coding sequences for the Cas12 protein and a guide polynucleotide flanked by inverted terminal repeats (ITRs).

In some embodiments, the CRISPR-Cas12 system is packaged into the AAV vector, such as AAV1, AAV2, AAV3, AAV4, AAV5, AAV6, AAV7, AAV8, AAV9, or AAVrh74. In some embodiments, the CRISPR-Cas12 system described herein is packaged into the AAV vector including an engineered capsid with tissue tropism, such as an engineered muscle-tropic capsid. Taboada et al., Cell 184:4919-4938 (2021) described the engineering of tissue-tropic AAV capsids through directed evolution and identifying a class of capsids containing an RGD motif, and systemic injection of MyoAAV enables efficient transduction of muscle tissue in non-human primates. The entire contents of this reference are incorporated herein by reference.

### Lipid Nanoparticle

Some embodiments of the present disclosure relate to a lipid nanoparticle (LNP) comprising the CRISPR-Cas12 system, and the LNP comprises the guide polynucleotide and mRNA encoding the Cas12 protein as described herein.

The LNP delivery of the CRISPR-Cas system was described in Gillmore et al., N. Engl. J. Med. 385:493-502 (2021). The LNP is composed of four lipids, including a proprietary ionizable lipid LP000001, DSPC, cholesterol, and DMG-PEG2k. An LNP suspension is formulated in an aqueous buffer including Tris, NaCl, and sucrose at pH of 7.4. The entire contents of this reference are incorporated herein by reference. In some embodiments, in addition to RNA payload (Cas12 mRNA and a guide polynucleotide), the LNP further comprises four components: a cationic or ionizable lipid, cholesterol, a helper lipid, and a PEG-lipid. In some embodiments, the cationic or ionizable lipid comprises cKK-E12, C12-200, ALC-0315, DLin-MC3-DMA, DLin-KC2-DMA, FTT5, Moderna SM-102, and Intellia LP01. In some embodiments, the PEG-lipid comprises PEG-2000-C-DMG, PEG-2000-DMG, or ALC-0159. In some embodiments, the helper lipid comprises DSPC. The components of LNP were described in Panuska et al., Nature Reviews Genetics 23:265-280 (2022). FDA-approved LNP comprises mutants of four basic components: a cationic or ionizable lipid, cholesterol, a helper lipid, and polyethylene glycol (PEG) lipid. The entire contents of this reference are incorporated herein by reference.

### Lentiviral Vector

Some embodiments of the present disclosure relate to a lentiviral vector comprising the CRISPR-Cas12 system described herein, and the lentiviral vector comprises the guide polynucleotide and mRNA encoding the Cas12 protein described herein. In some embodiments, the lentiviral vector is pseudotyped with homologous or heterologous envelope proteins such as VSV-G. In some embodiments, the mRNA encoding the Cas12 protein is linked to an aptamer sequence.

### Ribonucleoprotein (RNP) Complex

Some embodiments of the present disclosure relate to a RNP complex comprising the CRISPR-Cas12 system, and the RNP complex is formed by the guide polynucleotide and the Cas12 protein described herein. In some embodiments, the RNP complex may be delivered into eukaryotic cells, mammalian cells, or human cells via microinjection or electroporation. In certain embodiments, the RNP complex may be packaged into virus-like particles and delivered in vivo to mammalian or human subjects.

### Virus-Like Particle (VLP)

Some embodiments of the present disclosure relate to a VLP comprising the CRISPR-Cas12 system, and the VLP comprises the guide polynucleotide and the Cas12 protein described herein, or the RNP complex formed by the guide polynucleotide and the Cas12 protein.

The development and application of DNA-free virus-like particles (eVLPs) for efficient packaging and delivery of base editors or Cas9 ribonucleoproteins was described in Banskota et al., Cell 185(2):250-265 (2022). Mangeot et al., Nature Communications 10(1):1-15 (2019) revealed engineered murine leukemia virus-like particles (Nanoblades) loaded with Cas9-sgRNA ribonucleoproteins to induce efficient genome editing in cell lines and primary cells (including human induced pluripotent stem cells, human hematopoietic stem cells, and mouse bone marrow cells). Campbell et al., Molecular Therapy 27:151-163 (2019) revealed specialized extracellular vesicles called “gesicles” to efficiently yet transiently deliver Cas9 ribonucleoproteins targeting the HIV long terminal repeat (LTR) sequence. Gesicles are produced by expressing vesicular stomatitis virus glycoprotein and packaging proteins (as their cargo), thus eliminating the need for transgenic delivery and enabling more precise control over Cas9 expression. Mangeot et al., Molecular Therapy 19(9):1656-1666 (2011) revealed that overexpression of the vesicular stomatitis virus glycoprotein (VSV-G) in human cells induces the release of fusogenic vesicles (named gesicles). Biochemical and functional studies showed that glial cells incorporate proteins from producer cells and can deliver them to recipient cells. This protein transduction method enables the direct transfer of cytoplasmic, nuclear, or surface proteins in target cells. These references all describe engineered VLPs, the entire contents of each of which are incorporated herein by reference.

In some embodiments, the engineered VLP is pseudotyped with homologous or heterologous envelope proteins such as VSV-G. In some embodiments, the Cas12 protein is fused to a gag protein (e.g., MLV gag) via a cleavable linker, and cleavage of the linker in the target cell exposes a nuclear localization signal (NLS) located between the linker and the Cas12 protein. In some embodiments, the fusion protein or conjugate comprises (e.g., from the 5′ end to the 3′ end) the gag protein (e.g., MLV gag), one or more nuclear export signals (NES), a cleavable linker, one or more NLS, and Cas12, as described in Banskota et al., Cell 185(2):250 265 (2022).

In some embodiments, the Cas12 protein is fused to a first dimerization domain that is capable of dimerizing or heterodimerizing with a second dimerization domain fused to a membrane protein, and the presence of a ligand promotes such dimerization and facilitates the enrichment of the Cas12 protein or the fusion protein or conjugate thereof into the VLP, as described in Campbell et al., Molecular Therapy 27:151-163 (2019).

### Cell

Some embodiments of the present disclosure relate to a cell comprising the CRISPR-Cas12 system described herein. The cell (e.g., used to generate a cell-free system) may be prokaryotic or eukaryotic. For example, the cell comprises, but is not limited to, bacteria, archaea, plant, fungi, yeast, insect, and mammalian cell, such as Lactobacillus, Lactococcus, Bacillus (e.g., B. subtilis), Escherichia (e.g., Escherichia coli), Clostridium, Saccharomyces or Pichia (e.g., Saccharomyces cerevisiae or Pichia pastoris), Kluyveromyces lactis, Salmonella typhimurium, Drosophila cells, Caenorhabditis elegans cell, Xenopus laevis cell, SF9 cells, C129 cells, HEK293 cells, Neurospora, and immortalized mammalian cell line (e.g., HeLa, bone marrow cell line, and lymphoid cell line).

In some embodiments, the cell is a prokaryotic cell, such as a bacterial cell (e.g., Escherichia coli). In some embodiments, the cell is a eukaryotic cell, such as a mammalian or human cell. In some embodiments, the cell is a primary eukaryotic cell, a stem cell, a tumor/cancer cell, a circulating tumor cell (CTC), a blood cell (e.g., T cell, B cell, NK cell, regulatory T cell (Treg), etc.), a hematopoietic stem cell, a specialized immune cell (e.g., tumor-infiltrating lymphocyte or tumor-suppressive lymphocyte), or a stromal cell in the tumor microenvironment (e.g., cancer-associated fibroblast). In some embodiments, the cell is a brain or neuronal cell of the central or peripheral nervous system (e.g., neuron, astrocyte, microglial cell, retinal ganglion cell, rod or cone cell).

### Target Nucleic Acid or Target DNA

In some embodiments, the target nucleic acid is a target DNA.

The CRISPR-Cas12 system described herein may be used to target one or more target nucleic acid molecules, such as target nucleic acid molecules present in biological samples or environmental samples (e.g., soil, air, or water samples).

In some embodiments of the present disclosure, the target nucleic acid is a gene associated with a disease or disorder. In some embodiments, the target nucleic acid is a disease-associated gene. In some embodiments, the disease-associated gene is a pathogenic gene that directly causes the disease. In some embodiments, the disease-associated gene is an aberrant gene that directly causes the disease or a gene exhibiting abnormal expression. For example, the gene undergoes deleterious mutations, leading to occurrence of disease. As another example, the gene may be overexpressed or underexpressed, resulting in occurrence of disease. In some embodiments, overexpression of the gene leads to disease. In some embodiments, underexpression of the gene leads to disease. In some embodiments, the overexpression of the gene is associated with the occurrence of disease. In some embodiments, the underexpression of the gene is associated with the occurrence of disease.

In some embodiments of the present disclosure, the disease or disorder is a hematologic disease or disorder, an ophthalmic disease or disorder, a neurological disease or disorder, a respiratory disease or disorder, a hepatic disease or disorder, a metabolic disease or disorder, a cancer, or an infectious disease.

In some embodiments of the present disclosure, the target nucleic acid is selected from any one of the genes listed in Table 27, and the disease or disorder is listed in Table 27. Table 27 shows target nucleic acids and a disease or disorder corresponding to each target nucleic acid.

In some embodiments of the present disclosure, the disease or disorder is selected from: hemophilia A, Best yolk-like macular dystrophy, B-cell acute lymphoblastic leukemia, hemophilia B, CDKL5 deficiency, CLN2 disease, Niemann-Pick disease type C, Dravet syndrome, FOXG1 syndrome, GM1 ganglioside storage disease, GM2 ganglioside deposition disease, HIV infection, HSV infection, Usher syndrome type IB, Usher syndrome type IIA, Mucopolysaccharidosis type IIIA, Mucopolysaccharidosis type IIIB, Gaucher disease type III, Mucopolysaccharidosis type II, type II diabetes, Mucopolysaccharidosis type IV, Gaucher disease type I, Mucopolysaccharidosis type I, type I diabetes, Usher syndrome type I, KCNQ2 epileptic encephalopathy, Leber hereditary optic neuropathy, Leigh syndrome, Prader-Willi syndrome, SLC13A5 deficiency, X-linked myotubular myopathy, X-linked retinoschisis, X-linked retinitis pigmentosa, α1-antitrypsin deficiency, α-mannoside storage disease, α-thalassemia, β-thalassemia, Alzheimer's disease, Bardet-Biedl syndrome, white dot retinal degeneration, leukocyte adhesion deficiency type I, galactosemia, bladder cancer, overactive bladder, phenylketonuria, nasopharyngeal carcinoma, Bietti's crystalline dystrophy, pyruvate kinase deficiency, erectile dysfunction, autosomal recessive congenital ichthyosis, adult glucan body disease, traumatic arthritis, homozygous familial hypercholesterolemia, Fragile X syndrome, thalassemia, hypophosphatasia, epilepsy, multiple myeloma, multiple system atrophy, frontotemporal dementia, catecholamine-sensitive polymorphic ventricular tachycardia, Fabry's disease, Fanconi's anemia, aromatic L-amino acid decarboxylase deficiency, radiation-induced xerostomia, non-Hodgkin's lymphoma, non-muscle invasive bladder carcinoma, non-alcoholic fatty liver disease, non-small cell lung cancer, hypertrophic cardiomyopathy, hypertrophic scar, obesity, peroneal muscular dystrophy type 1A, peroneal muscular dystrophy type 2A, pulmonary hypertension, Friedrich's ataxia, peritoneal carcinoma, liver cancer, hepatocellular carcinoma, dry age-related macular degeneration, sicca syndrome, hyperuricemia, hyperlipidemia, Gaucher disease, autism spectrum disorders, osteoarthritis, bone marrow failure syndromes, citrullinemia type I, coronary heart disease, cystinosis, melanoma, Huntington's disease, amyotrophic lateral sclerosis, urge incontinence, acute intermittent porphyria, acute lymphoblastic leukemia, spinal cerebellar ataxia, spinal muscular atrophy with respiratory distress type 1, spinal muscular atrophy, Tay-Sachs disease, methylmalonic acidemia, thyroid carcinoma, pseudohypertrophic muscular dystrophy, anaplastic astrocytoma, intermittent claudication, junctional epidermolysis bullosa, glioma, glioblastoma, corneal graft rejection, colorectal cancer, progressive multifocal leukoencephalopathy, progressive familial intrahepatic cholestasis, giant-axonal neuropathy, Canavan's disease, cocaine addiction, Klaber's disease, Kriegler-Najjar syndrome, oral cancer, Angelman syndrome, diffuse intrinsic pontine glioma, Lafora's disease, rheumatoid arthritis, sickle cell disease, lymphedema, ovarian cancer, chronic lymphocytic leukemia, chronic granulomatous disease, chronic nephrogenic anemia, chronic pain, chronic hepatitis B, Menkes' disease, cystic fibrosis, Netherseton's syndrome, ornithine transcarbamylase deficiency, Parkinson's disease, Pompe's disease, uveitis, prostate cancer, vestibular schwannoma, ankylosing muscular dystrophy, ankylosing spondylitis, castration-resistant prostate cancer, glaucoma, achromatopsia, ischemic heart failure, lysosomal storage disease, sarcoma, breast cancer, Rett's syndrome, triple-negative breast cancer, Sandhoff's disease, color blindness, heart failure with reduced ejection fraction, neuronal ceroid lipofuscinosis, adrenoleukodystrophy, renal cell carcinoma, wet age-related macular degeneration, eczema, thrombocytopenia with immunodeficiency syndrome, esophageal cancer, optic neuropathy, optic nerve atrophy, retinal vein occlusion, retinitis pigmentosa, rhodopsin-mediated autosomal dominant retinitis pigmentosa, ependymoma, fallopian tube carcinoma, bilateral vestibulopathies, Stargardt's disease, diabetic macular edema, diabetic neuropathy, diabetic retinopathy, diabetic peripheral neuralgia, diabetic foot, glycogenosis, glycogenosis type Ia, glycogenosis type IIb, atopic dermatitis, hearing loss, hearing impairment, head and neck cancer, squamous cell carcinoma of the head and neck, Wilson's disease, stable angina pectoris, Usher's syndrome, choroideremia, Leber's congenital amaurosis, congenital adrenal hyperplasia, cardiomyopathy, angina pectoris, heart failure, COVID-19 infection, pleural mesothelioma, acne vulgaris, severe combined immunodeficiency diseases, severe limb ischemia, oculopharyngeal muscular dystrophy, pancreatic cancer, graft-versus-host disease, hereditary retinal dystrophy, hereditary angioedema, hepatitis B, heterotrophic cerebral leukoencephalic dystrophy, psoriatic arthritis, recessive genetic dystrophic epidermolysis bullosa, infantile malignant osteosclerosis, dystrophic epidermolysis bullosa, morphea, primary immune deficiency, heterozygous familial hypercholesterolemia, limb-girdle muscular dystrophy type 2B, limb-girdle muscular dystrophy type 2C, limb-girdle muscular dystrophy type 2D, limb-girdle muscular dystrophy type 2E, limb-girdle muscular dystrophy type 2I, limb-girdle muscular dystrophy type 2L, limb ischemic disease, lipoprotein lipase deficiency, severe congenital neutrophilic dysphoria, wrinkles, stroke, sciatica, schizophrenia, depression, drug addiction, autism, idiopathic pulmonary fibrosis, hyperlipidemia, transthyretin (ATTR) amyloidosis, alpha-1-antitrypsin deficiency (AATD) liver disease, and AATD lung disease.

Genes associated with ATTR amyloidosis comprise, but are not limited to, ATTR.

Genes associated with Leber hereditary optic neuropathy comprise, but are not limited to, MT-ND4.

Genes associated with AATD liver disease comprise, but are not limited to, AATD.

Genes associated with AATD lung disease comprise, but are not limited to, AATD.

Genes associated with the graft-versus-host disease comprise, but are not limited to, thymidine kinase genes.

Genes associated with hereditary retinal dystrophy comprise, but are not limited to, RPE65.

Genes associated with spinal muscular atrophy comprise, but are not limited to, SMN1.

Genes associated with osteoarthritis comprise, but are not limited to, TGF-81.

Genes associated with hemophilia A comprise, but are not limited to, factor VIII.

Genes associated with hemophilia B comprise, but are not limited to, factor IX.

Genes associated with cystic fibrosis comprise, but are not limited to, CFTR.

Genes associated with Parkinson's disease comprise, but are not limited to, Gad1, Gad2, PTBP1, KEAP1, RE1, Amigo1, Gprc5c, Let-7a, Pnky, LRRK2, SNCA, GBA, miR-92b, miR-9, miR-124, miR-181, HMGB1, TRIM72, GPNMB, and REST.

Genes associated with Usher syndrome comprise, but are not limited to, USH2A.

Genes associated with α-thalassemia, β-thalassemia, and the sickle cell disease comprise, but are not limited to, BCL11A, HBG, HBA, and HBB.

Genes associated with pulmonary hypertension comprise, but are not limited to, eNOS.

Genes associated with Stargardt's disease comprise, but are not limited to, ABCA4.

Genes associated with age-related macular degeneration comprise, but are not limited to, VEGFA, VEGFR, IL17, Kir7.1, LCN-2, IRAK-M, CD59, LTA4H, GPX4, GLS1, PAPP-A, cGAS, STING, mTOR, GCN2, Nrf2, Ang 2, CTGF, Complement C3, Complement C5, CHFR4b, DOCK6, CTSS, ELN, and FGF2.

Genes associated with glaucoma comprise, but are not limited to, AQP1, ADRB2, NMNTA2, NRP1, Hrh1, Anxa2, OPA1, Cx43, ANGPTL7, MYOC, ROCK1, ROCK2, TIMP1, TIMP2, TIMP3, TIMP4, carbonic anhydrase CA2, carbonic anhydrase CA4, and carbonic anhydrase CA12.

Genes associated with idiopathic pulmonary fibrosis comprise, but are not limited to, CTGF.

Genes associated with hyperlipidemia comprise, but are not limited to, PCSK9.

Genes associated with Alzheimer's disease comprise, but are not limited to, NGF.

Genes associated with coronary heart disease comprise, but are not limited to, VEGFA and bFGF.

Genes associated with chronic nephrogenic anemia related comprise, but are not limited to, EPO.

Genes associated with Leber's congenital amaurosis comprise, but are not limited to, RPE65.

Genes associated with retinitis pigmentosa comprise, but are not limited to, PDE6B.

Genes associated with phenylketonuria comprise, but are not limited to, PAH.

Genes associated with epilepsy comprise, but are not limited to, GATI.

In some embodiments of the present disclosure, a sequence of the target nucleic acid is as shown in any one of sequences shown in SEQ ID NO: 761-782.

Non-limiting examples of the target nucleic acid also include target nucleic acids disclosed in U.S. Provisional Patent Application No. 61/736,527, filed on Dec. 12, 2012, U.S. Provisional Patent Application No. 61/748,427, filed on Jan. 2, 2013, and International Application No. PCT/US2013/074667, filed on Dec. 12, 2013, the entire contents of each of which are incorporated herein by reference.

In some embodiments, the target nucleic acid is a reporter gene. Examples of the reporter gene include, but are not limited to, glutathione-S-transferase (GST), horseradish peroxidase (HRP), chloramphenicol acetyltransferase (CAT), β-galactosidase, β-glucuronidase, luciferase, green fluorescent protein (GFP), HcRed, DsRed, cyan fluorescent protein (CFP), yellow fluorescent protein (YFP), and autofluorescent protein including blue fluorescent protein (BFP).

### Use for Treatment or Prevention of Disease

Some embodiments of the present disclosure relate to a pharmaceutical composition comprising the Cas12 protein, the guide polynucleotide, the inactivated Cas12 mutant, the fusion protein or conjugate, the isolated nucleic acid, the CRISPR-Cas12 system, the vector system, the delivery system, or the cell, which are all described in the present disclosure. For example, the pharmaceutical composition may comprise an AAV vector encoding the Cas12 protein or the inactivated Cas12 mutant and the guide polynucleotide. For example, the pharmaceutical composition may comprise a lipid nanoparticle comprising the guide polynucleotide and mRNA encoding the Cas12 protein. For example, the pharmaceutical compositions may comprise a lentiviral vector comprising the guide polynucleotide and the mRNA encoding the Cas12 protein. For example, the pharmaceutical composition may comprise a virus-like particle comprising the guide polynucleotide and the Cas12 protein, or a ribonucleoprotein complex formed by the guide polynucleotide and the Cas12 protein.

Some embodiments of the present disclosure relate to use of the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit described herein in cleaving or editing a target nucleic acid in a mammalian cell.

Some embodiments of the present disclosure relate to use of the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit described herein in any of the following: cleaving one or more target nucleic acid molecules or introducing nicks into the one or more target nucleic acid molecules, activating or upmodulating an expression of the one or more target nucleic acid molecules, activating or inhibiting transcription of the one or more target nucleic acid molecules, inactivating the one or more target nucleic acid molecules, visualizing, labeling, or detecting the one or more target nucleic acid molecules, binding the one or more target nucleic acid molecules, transporting the one or more target nucleic acid molecules, and masking the one or more target nucleic acid molecules.

Some embodiments of the present disclosure relate to use of the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit described herein in modifying one or more target nucleic acid molecules, and the modifying one or more target nucleic acid molecules comprises one or more of: nucleic acid base substitution, nucleic acid base deletion, nucleic acid base insertion, breakage of a target nucleic acid, nucleic acid methylation, and nucleic acid demethylation.

Some embodiments of the present disclosure relate to use of the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit described herein in diagnosing, treating, or preventing a disease or disorder associated with the target nucleic acid.

Some embodiments of the present disclosure relate to use of the Cas12 protein, the guide polynucleotide, the inactivated Cas12 mutant, the fusion protein or conjugate, the isolated nucleic acid, the CRISPR-Cas12 system, the vector system, the delivery system, the cell, the pharmaceutical composition, or the kit described herein in preparing a medicament for diagnosing, treating, or preventing a disease or disorder associated with the target nucleic acid.

In some embodiments of the present disclosure, the target nucleic acid is optionally selected from genes as listed in Table 27, and the disease or disorder is as listed in Table 27. Table 27 shows target nucleic acids and a disease or disorder corresponding to each target nucleic acid.

In some embodiments of the present disclosure, specific genes, such as those listed in Table 27, are subjected to targeted cleavage by the CRISPR-Cas12 system, thereby preventing, diagnosing, or treating the corresponding disease or disorder in Table 27. After targeted cleavage, indels are introduced through cellular repair, resulting in the knockout of the target gene, thereby suppressing its function.

In some embodiments of the present disclosure, specific genes, such as those listed in Table 27, are subjected to targeted modification by the CRISPR-Cas12 system, thereby preventing, diagnosing, or treating the corresponding disease or disorder in Table 27.

In some embodiments of the present disclosure, an expression of specific genes, such as those listed in Table 27, is subjected to targeted modulation by the CRISPR-Cas12 system, thereby preventing, diagnosing, or treating the corresponding disease or disorder in Table 27.

In some embodiments, the pharmaceutical composition is delivered in vivo to a human subject. The pharmaceutical composition may be delivered by any effective route. Exemplary routes of administration include, but are not limited to, intravenous infusion, intravenous injection, intraperitoneal injection, intramuscular injection, intratumoral injection, subcutaneous injection, intradermal injection, intraventricular injection, intravascular injection, intracerebellar injection, intraocular injection, subretinal injection, intravitreal injection, intracameral injection, intratympanic injection, intranasal injection, and inhalation.

### Diagnostic Application

Some embodiments of the present disclosure relate to an in vitro composition, comprising the CRISPR-Cas12 system described herein and a marked detector DNA that does not hybridize with the guide polynucleotide described herein.

Some embodiments of the present disclosure relate to the use of the CRISPR-Cas12 system in detecting a target nucleic acid in a nucleic acid sample suspected of containing a target nucleic acid.

Some embodiments of the present disclosure relate to the use of the CRISPR-Cas12 system in detecting a target nucleic acid in a nucleic acid sample containing the target nucleic acid.

In some embodiments, the detected target nucleic acid is a target RNA.

In some embodiments, the detected target nucleic acid is a target DNA. In some embodiments, a method for detecting the target DNA comprises fusing a Cas12 protein to a fluorescent protein or other detectable marker and a guide sequence of a guide polynucleotide being specific to the target DNA. The binding of Cas12 to the target DNA may be visualized by microscopy or other imaging manners.

In some embodiments, a method for detecting a target nucleic acid in a cell-free system results in the generation of a detectable marker or enzymatic activity. For example, by using the Cas12 protein, the guide polynucleotide comprising the guide sequence specific to the target DNA, and the detectable marker, the target nucleic acid may be recognized by Cas12. Binding of Cas12 to the target nucleic acid triggers its DNase activity, which results in cleavage of the target nucleic acid and the detectable marker.

In some embodiments, the detectable marker is DNA linked to a fluorescent probe and a quencher. The complete detectable DNA ligates to the fluorescent probe and quencher, suppressing fluorescence. After the detectable DNA is cleaved by Cas12, the fluorescent probe is released from the quencher and exhibits fluorescent activity. This method may be used to determine whether the target DNA is present in lysed cell samples, lysed tissue samples, blood samples, saliva samples, environmental samples (e.g., water, soil, or air samples), or other lysed cell or cell-free samples. This method may also be used to detect pathogens such as viruses or bacteria, or to diagnose disease states such as cancer.

In some embodiments, the detection of the target nucleic acid is conducive to diagnosing a disease and/or pathological condition, or the presence of viral or bacterial infection.

| Target site (gene) | Disease name |
|---|---|
| 17β-HSD13 | Non-alcoholic steatohepatitis |
| | (NASH) |
| 17β-HSD13 | Metabolic |
| | dysfunction-associated fatty |
| | liver disease (MAFLD) |
| 17β-HSD5, | Castration-resistant prostate |
| PSMA | cancer |
| 23S rRNA | Klebsiella pneumoniae |
| | infection |
| 4-1BB, | Refractory plasma cell |
| BCMA | myeloma |
| 4-1BB, | Relapsed Multiple myeloma |
| BCMA | |
| 4-1BB, | Multiple myeloma |
| BCMA | |
| 4-1BB, CD19 | Myasthenia gravis |
| 4-1BB, CD19 | Small lymphocytic |
| | lymphoma |
| 4-1BB, CD19 | Systemic sclerosis |
| 4-1BB, CD19 | Systemic lupus |
| | erythematosus |
| 4-1BB, CD19 | Idiopathic inflammatory |
| | myopathy |
| 4-1BB, CD19 | Dermatomyositis |
| HLA | Head and neck tumor |
| HLA | Hemophagocytic |
| | lymphohistiocytosis |
| HLA | Breast cancer |
| HLA | Prostate cancer |
| HLA | Diffuse large B-cell |
| | lymphoma |
| HLA | Ovarian cancer |
| HLA | Colorectal cancer |
| HLA | Refractory plasma cell |
| | myeloma |
| HLA | Acquired immunodeficiency |
| | syndrome (AIDS) |
| HLA | Melanoma |
| HLA | Relapsed Multiple myeloma |
| HLA | Lung cancer |
| HLA | Bladder cancer |
| 4-1BB, CD19 | Chronic lymphocytic |
| | leukemia |
| 4-1BB, CD19 | Lymphocytic leukemia |
| 4-1BB, CD19 | Lupus nephritis |
| 4-1BB, CD19 | Non-Hodgkin lymphoma |
| 4-1BB, | Chronic lymphocytic |
| CD19, CD3ζ, | leukemia (CLL) |
| EGFR | |
| 4-1BB, | Acute lymphoblastic |
| CD19, CD3ζ, | leukemia (ALL) |
| EGFR | |
| 4-1BB, | Non-Hodgkin lymphoma |
| CD19, CD3ζ, | |
| EGFR | |
| 4-1BB, CD40 | Biliary tract tumor |
| 4-1BB, | Metastatic hepatocellular |
| CD40L, | carcinoma |
| CTLA4 | |
| 4-1BB, | Microsatellite-stable |
| CD40L, | colorectal cancer (MSS CRC) |
| CTLA4 | |
| 4-1BB, | Advanced malignant solid |
| CD40L, | tumor |
| CTLA4 | |
| 4-1BB, | Solid tumor |
| CD40L, | |
| CTLA4 | |
| 4-1BB, | Locally advanced head and |
| CD40L, | neck squamous cell |
| CTLA4 | carcinoma (HNSCC) |
| 4-1BB, | Locally advanced |
| CD40L, | hepatocellular carcinoma |
| CTLA4 | (HCC) |
| HLA | Epstein-Barr virus infection |
| HLA class | Tumor |
| II antigen, | |
| MAGEA3 | |
| HLA class | Solid tumor |
| II antigen, | |
| TERT | |
| HLA class I | Myxoid liposarcoma |
| antigen | |
| HLA class I | Synovial sarcoma |
| antigen | |
| HLA class I | Non-small cell lung cancer |
| antigen | (NSCLC) |
| HLA class I | Type 1 diabetes |
| antigen, | |
| HLA class | |
| II antigen | |
| HLA class I | Solid tumor |
| antigen, | |
| KKLC1 | |
| HLA, | Malignant epithelial tumor |
| MAGEA3 | |
| HLA, | Tumor |
| NY-ESO-1, | |
| transforming | |
| growth | |
| factor β | |
| (TGFβ) | |
| receptor | |
| HLA, | Tumor |
| TGFBR2 | |
| HLA-A2 | Renal transplant rejection |
| HLA-A2 | Liver transplant rejection |
| HLA-A2 | Liver failure |
| 4-1BB, | Recurrent head and neck |
| CD40L, | squamous cell carcinoma |
| CTLA4 | (Recurrent HNSCC) |
| 4-1BB, EBV | Nasopharyngeal carcinoma |
| protein | (NPC) |
| 4-1BB, EBV | Niemann-Pick disease type C |
| protein | |
| 4-1BB, HPV | Advanced cancer |
| E7, IL2RA | |
| 4-1BB, HPV | Head and neck tumor |
| E7, IL2RA | |
| 4-1BB, HPV | Human papillomavirus type |
| E7, IL2RA | 16 positive (HPV-16 positive) |
| 4-1BB, HPV | Cervical cancer |
| E7, IL2RA | |
| 4-1BB, HPV | Anal tumor |
| E7, IL2RA | |
| 4-1BB, HPV | HPV16 infection |
| E7, IL2RA | |
| 4-1BB, | Advanced malignant solid |
| IL-12R | tumor |
| 4-1BB, | Head and neck squamous cell |
| IL-12R | carcinoma (HNSCC) |
| 4-1BB, | Triple-negative breast cancer |
| IL-12R | (TNBC) |
| 4-1BB, | Cutaneous melanoma |
| IL-12R | |
| 4-1BB, | Urothelial carcinoma |
| IL-12R | |
| 4-1BB, | Melanoma |
| IL-12R | |
| 4-1BB, | Non-small cell lung cancer |
| IL-12R | (NSCLC) |
| 4-1BB, | Bladder urothelial carcinoma |
| IL-12R | |
| 4-1BBL, | Tumor |
| CD19, PSCA | |
| 4-1BBL, | Solid tumor |
| IL15R | |
| 5T4 | Solid tumor |
| 5T4 | Locally advanced malignant |
| | solid tumor |
| 5T4 | Acute myeloid leukemia |
| | (AML) |
| HLA-A2, | Triple-negative breast |
| NY-ESO-1 | cancer (TNBC) |
| HLA-A2, | Sarcoma |
| NY-ESO-1 | |
| HLA-G | Metastatic clear cell renal |
| | cell carcinoma |
| HLA-G | Hematologic malignancy |
| HLA-G | Solid tumor |
| HLA-G | Renal cell carcinoma |
| HLA-G | Ovarian cancer |
| HLA-G | Locally advanced clear cell |
| | renal cell carcinoma |
| HLA-G | Acute myeloid leukemia |
| | (AML) |
| HMGB1 | Fibrosis |
| HMGB1 | Pain |
| HMGB1 | Sepsis |
| HMGB1 | Pulmonary fibrosis |
| HP-NAP | Breast cancer |
| HPV E6 | Cervical cancer |
| HPV E6, | HPV16-positive solid tumor |
| HPV E7 | |
| HPV E7 | Cervical dysplasia |
| HPV E7 | Tumor metastasis |
| HPV E7 | Vaginal adenocarcinoma |
| HPV E7 | Novel coronavirus infection |
| HPV E7 | Vulvar tumor |
| HPV E7 | Vulvar intraepithelial |
| | neoplasia (VIN) |
| 5T4 | Unresectable malignant solid |
| | tumor |
| A1AT | Liver fibrosis |
| A1AT | Liver disease |
| A1AT | Scar |
| A1AT | Alpha-1 antitrypsin |
| | deficiency |
| A1R | Asthma |
| ABCA4 | Mucopolysaccharidosis type I |
| | (MPS I) |
| ABCA4 | Cystic fibrosis |
| ABCA4 | Huntington's disease |
| ABCA4 | Alzheimer's disease |
| ABCA4 | Stargardt disease type 4 |
| ABCA4 | Friedreich's ataxia |
| ABCD1 | Adrenoleukodystrophy |
| | (ALD) |
| ABCD1 | Hypertriglyceridemia |
| AC | Heart failure |
| ACE2 | Primary sclerosing |
| | cholangitis |
| ACE2 | Coronavirus infection |
| AChE | Myasthenia gravis |
| AChE | Inflammatory bowel disease |
| AChE | Amyotrophic lateral sclerosis |
| ACTG2 | Megacystis-microcolon-intestinal |
| | hypoperistalsis syndrome |
| | (MMIHS) |
| ACVR2B | Cachexia |
| ADA | Severe combined |
| | immunodeficiency |
| ADA | Adenosine deaminase |
| | deficiency |
| ADAM8 | Tumor |
| ADAMTS5 | Osteoarthritis |
| ADAMTS5, | Rheumatoid arthritis |
| TNF | |
| ADAMTS5, | Osteoarthritis |
| HPV E7 | Head and neck tumor |
| HPV E7 | Papillomavirus infection |
| HPV E7 | HPV-associated vulvar |
| | squamous cell carcinoma |
| HPV E7 | HPV-associated penile |
| | squamous cell carcinoma |
| HPV E7 | HPV-associated cervical |
| | cancer |
| HPV E7 | Squamous cell tumor |
| HPV E7 | Squamous intraepithelial |
| | lesion (SIL) |
| HPV E7 | Oropharyngeal tumor |
| HPV E7 | Glioblastoma |
| HPV E7 | Spinal cord injury |
| HPV E7 | Macular degeneration |
| HPV E7 | Laryngeal tumor |
| HPV E7 | Cervical intraepithelial |
| | neoplasia (CIN) |
| HPV E7 | Cervical cancer |
| HPV E7 | Polyomavirus infection |
| HPV E7 | HPV-associated squamous |
| | cell carcinoma |
| HPV E7 | HPV16-positive solid tumor |
| HS | Mucopolysaccharidosis type |
| | III (MPS III) |
| Hsp27 | Pancreatic cancer |
| Hsp27 | Prostate cancer |
| Hsp27 | Non-small cell lung cancer |
| | (NSCLC) |
| Hsp27 | Bladder cancer |
| Hsp47 | Fibrosis |
| Hsp47 | Systemic sclerosis |
| Hsp47 | Idiopathic pulmonary |
| | fibrosis |
| HSP70 | Non-small cell lung cancer |
| heat-shock | (NSCLC) |
| proteins | |
| HSPA1A, | Tumor |
| p53 | |
| HSPA9 | Ovarian cancer |
| ADAR | Novel coronavirus infection |
| ADAR | Decompensated liver |
| | cirrhosis |
| ADAR | Immunoglobulin A |
| | nephropathy |
| ADARB1 | Amyotrophic lateral sclerosis |
| ADP | Tumor |
| ADP, | Pancreatic cancer |
| thymidine | |
| kinase | |
| aFGF | Peripheral arterial occlusive |
| | disease |
| aFGF | Eczema |
| aFGF | Chronic limb-threatening |
| | ischemia |
| aFGF | Intermittent claudication |
| aFGF | Arterial occlusive disease |
| AFP | Liver cancer |
| AGER | Inflammation |
| AGER | Asthma |
| AGL | Glycogen storage disease |
| | type III (GSD III) |
| AGRE2, | Relapsed acute myeloid |
| CLL-1 | leukemia |
| AGT | Preeclampsia |
| AGT | Heart failure with reduced |
| | ejection fraction |
| AGT | Chronic heart failure |
| AGT | Hypertension |
| HSPGs, MMPs, | Tumor |
| TGF-β1 | |
| HSPGs, MMPs, | Trauma and injury |
| TGF-β1 | |
| HTATIP2 | Complement disorder |
| HTT | Hodgkin lymphoma |
| HTT | Huntington's disease |
| Hyaluronic | Metastatic pancreatic |
| acid | adenocarcinoma |
| Hyaluronic | Secondary malignant |
| acid | neoplasm of pancreas |
| Hyaluronic | Retinoblastoma |
| acid | |
| Hyaluronic | Brain cancer |
| acid | |
| Hyaluronic | Melanoma |
| acid | |
| Hypoxia- | Myocardial ischemia |
| inducible | |
| factor 1 | |
| Hypoxia- | Peripheral vascular disease |
| inducible | |
| factor 1 | |
| Hypoxia- | Peripheral arterial disease |
| inducible | |
| factor 1 | |
| Hypoxia- | Intermittent claudication |
| inducible | |
| factor 1 | |
| Hypoxia- | Atherosclerosis |
| inducible | |
| factor 1 | |
| ICAM-1 | Inflammatory bowel disease |
| ICAM-1 | Inflammation |
| ICAM-1 | Ulcerative colitis |
| ICAM-1 | Anaplastic thyroid |
| | carcinoma |
| ICAM-1 | Poorly differentiated thyroid |
| carcinoma | |
| AGT | Alzheimer's disease |
| AGT, | Cardiovascular disease |
| ANGPTL3 | |
| AGT, | Hypertension |
| APOC3 | |
| AGT, | Hypertriglyceridemia |
| APOC3 | |
| AIPL1 | Cone dystrophy |
| AIPL1 | Retinitis pigmentosa |
| AIPL1 | Leber congenital amaurosis |
| Akt, PTEN | Tumor |
| Akt-1 | Metastatic renal cell |
| | carcinoma |
| Akt-1 | Secondary malignant |
| | neoplasm of pancreas |
| Akt-1 | Pancreatic cancer |
| Akt-1 | Advanced renal cell |
| | carcinoma |
| Akt-1 | Advanced hepatocellular |
| | carcinoma |
| Akt-1 | Advanced malignant solid |
| | tumor |
| Akt-1 | Renal cell carcinoma |
| ALAS1 | Acute hepatic porphyria |
| ALAS1 | Hepatic porphyria |
| ALDH2 | Esophageal cancer |
| ALDH2 | Alcohol use disorder |
| ALDH2 | Glioblastoma |
| ALDH2 | Glioma |
| ALDH2 | Osteoporosis |
| ALK5 | Ocular disease |
| ALPP | Endometrial cancer |
| ALPP | Ovarian cancer |
| ALPPL2 | Solid tumor |
| AMHR2 | Tumor |
| AML1-ETO | Acute myeloid leukemia |
| fusion protein | |
| Ang2 | Retinal disorder |
| Ang2 | Prostate cancer |
| Ang2 | Sepsis |
| Ang2 | Acute lung injury |
| Ang2 | Acute lung injury |
| ICAM-1 | Recurrent thyroid cancer |
| ICAM-1 | Non-small cell lung cancer |
| ICE1, | Neurofibroma |
| caspase | |
| ID1 | Tumor |
| IDS | Mucopolysaccharidosis type |
| | II |
| IDUA | Mucopolysaccharidosis type |
| | I |
| IFN α | Head and neck tumor |
| IFN α | Melanoma |
| IFNAR | Pleural effusion |
| IFNAR | Rheumatoid arthritis |
| IFNAR | Colorectal cancer |
| IFNAR | Glioblastoma |
| IFNAR | Non-muscle-invasive |
| | bladder tumor |
| IFNAR | Malignant pleural |
| | mesothelioma |
| IFNAR | Pleomorphic glioblastoma |
| IFNAR | Hepatitis C |
| IFNAR | Bladder cancer |
| IFNAR | Bladder cancer |
| IFNGR | Tumor |
| IFNGR | Heart failure |
| IFNGR | Perioperative ischemia |
| IFNGR | Peripheral vascular disease |
| IFNGR | Neurodegenerative disease |
| IFNGR | Cutaneous T-cell lymphoma |
| IFNGR | Candidemia |
| IFNGR | Brain injury |
| IFNGR | Basal cell nevus syndrome |
| IFNGR | Melanoma |
| IFNGR | Lung disease |
| IFNGR | Type 1 diabetes |
| IFNα2 | Melanoma |
| IFNβ | Endometrial cancer |
| IFNβ | Glioma |
| Ang2, VEGF | Wet age-related macular |
| | degeneration |
| Angiostatin | Corneal transplant rejection |
| ANGPTL3 | Primary |
| | hypercholesterolemia |
| ANGPTL3 | Hypertriglyceridemia |
| ANGPTL3 | Homozygous familial |
| | hypercholesterolemia |
| ANGPTL3 | Type II hyperlipoproteinemia |
| ANO5 | Limb-girdle muscular |
| | dystrophy |
| AP4M1 | Autosomal recessive spastic |
| | paraplegia type 50 |
| APN | B-cell lymphoma |
| APN, DPP-4 | Organ transplant rejection |
| APOA1 | Hypercholesterolemia |
| APOB | Heterozygous familial |
| | hypercholesterolemia |
| APOB | Neonatal disease |
| APOB | Congenital malformation |
| APOB | Coronary artery disease |
| APOB | Coronary heart disease |
| APOB | High and low density |
| | lipoprotein cholesterolemia |
| APOB | Hypercholesterolemia |
| APOB | Homozygous familial |
| | hypercholesterolemia |
| APOB | Type II hyperlipoproteinemia |
| APOB | Type IIa |
| | hyperlipoproteinemia |
| APOC3 | Combined lipase deficiencies |
| APOC3 | Dyslipidemia |
| APOC3 | Hemophilia B |
| APOC3 | Familial chylomicronemia |
| | syndrome |
| APOC3 | Hyperlipidemia |
| APOC3 | Hypertriglyceridemia |
| APOC3 | Non-alcoholic steatohepatitis |
| APOC3 | Atherosclerosis |
| IFNβ | Acute myeloid leukemia |
| IFNβ | Multiple sclerosis |
| IFNβ | Multiple myeloma |
| IFNβ | T-cell lymphoma |
| IFNβ, NIS | Neuroendocrine carcinoma |
| IFNβ, NIS | Refractory malignant solid |
| | tumor |
| IFNβ, NIS | Colorectal cancer |
| IFNβ, NIS | Acute myeloid leukemia |
| IFNβ, NIS | Liver cancer |
| IFNβ, NIS | Malignant solid tumor |
| IFNβ, NIS | Multiple myeloma |
| IFNβ, NIS | T-cell lymphoma |
| IFNβ, | Inflammation |
| TLR9 | |
| IFNγ | Castration-resistant prostate |
| | cancer |
| IFNγ, | Tumor |
| TERT | |
| IgE | Immune system disease |
| IgE | Hypersensitivity |
| IgE | Asthma |
| receptors | |
| IGF-1 | Tumor |
| IGF-1 | Alzheimer's disease |
| IGF-1 | Type 1 diabete |
| IGF-1R | Psoriasis |
| IGF-1R | Fibrosis |
| IGF-1R | Head and neck tumor |
| IGF-1R | Glaucoma |
| IGF-1R | Prostate cancer |
| IGF-1R | Ovarian cancer |
| IGF-1R | Glioblastoma |
| IGF-1R | Amyotrophic lateral |
| | sclerosis |
| APOC3 | Type I hyperlipoproteinemia |
| APOC3, | Dyslipidemia |
| PCSK9 | |
| APOC3, | Metabolic disease |
| PCSK9 | |
| APOC3, | Hemochromatosis |
| TMPRSS6 | |
| APOC3, | Hypertriglyceridemia |
| TMPRSS6 | |
| ApoE2 | Alzheimer's disease |
| APP | Down syndrome |
| APP | Cerebral amyloid angiopathy |
| APP | Alzheimer's disease |
| APRIL | Refractory plasma cell |
| | myeloma |
| APRIL | Relapsed Multiple myeloma |
| AQP1 | Parotid gland disease |
| AQP1 | Xerostomia |
| AR | Androgenic alopecia |
| AR | Castration-resistant prostate |
| | cancer |
| AR | Prostate cancer |
| AR | Prostate cancer |
| AR | Bone cancer |
| AR, mTOR | Benign prostatic hyperplasia |
| AR, mTOR | Prostate cancer |
| AR, PRLR, | Tumor |
| aromatase | |
| ARHGAP45 | Primary myelofibrosis |
| ARHGAP45 | Hematologic malignancy |
| ARHGAP45 | Solid tumor |
| ARHGAP45 | Chronic myeloid leukemia |
| ARHGAP45 | Lymphoma |
| ARHGAP45 | Acute myeloid leukemia |
| ARHGAP45 | Acute lymphoblastic |
| | leukemia |
| ARHGAP45 | Myelodysplastic syndrome |
| ARHGAP45 | Myelofibrosis |
| ARHGAP45 | Multiple myeloma |
| ASA | Metachromatic |
| | leukoencephalopathy |
| ASC2 | Inflammation |
| IGF-1R | Graves' ophthalmopathy |
| IGF-1R | Hepatocellular carcinoma |
| IGF-1R | Laron syndrome |
| IGFBP2, | Breast cancer |
| IGFBP5 | |
| IGHMBP2 | Charcot-Marie-Tooth |
| | disease type 2S |
| IKKγ, | Tumor |
| NF-κB | |
| IL-10R | Autoimmune hepatitis |
| IL-10R | Inflammation |
| IL-10R | Solid tumor |
| IL-10R | Amyotrophic lateral |
| | sclerosis |
| IL-10R | Musculoskeletal pain |
| IL-10R | Arthralgia |
| IL-10R | Multiple sclerosis |
| IL-10R | Back pain |
| IL-12 | Metastatic melanoma |
| IL-12 | Tumor |
| IL-12 | Pancreatic cancer |
| IL-12 | Adverse drug reaction |
| IL-12 | Novel coronavirus infection |
| IL-12 | Head and neck tumor |
| IL-12 | Head and neck tumor |
| IL-12 | Triple-negative breast |
| | cancer |
| IL-12 | Breast cancer |
| IL-12 | Prostate cancer |
| IL-12 | Skin tumor |
| IL-12 | Cutaneous T-cell lymphoma |
| IL-12 | Diffuse intrinsic pontine |
| | glioma |
| IL-12 | Merkel cell carcinoma |
| IL-12 | Ovarian cancer |
| IL-12 | Squamous cell carcinoma |
| IL-12 | Locally advanced melanoma |
| IL-12 | Glioblastoma |
| IL-12 | Acute myeloid leukemia |
| ASGR1, | End-stage renal disease |
| L-lactate | |
| dehydrogenase | |
| ASGR1, | Primary hyperoxaluria type 2 |
| L-lactate | |
| dehydrogenase | |
| ASGR1, | Primary hyperoxaluria type 1 |
| L-lactate | |
| dehydrogenase | |
| ASGR1, | Primary hyperoxaluria |
| L-lactate | |
| dehydrogenase | |
| ASN | Metastatic pancreatic ductal |
| | adenocarcinoma |
| ASN | Advanced pancreatic ductal |
| | adenocarcinoma |
| ASS | Citrullinemia |
| ASXL1, | Acute myeloid leukemia |
| RUNX1, p53 | |
| AT III | Hemophilia B |
| AT III | Hemophilia A |
| AT III | Venous thromboembolism |
| AT III | Atherosclerosis |
| AT III | Hemorrhage |
| ATM | Ataxia-telangiectasia |
| ATOH1 | Hearing loss |
| ATP7A | Menkes syndrome |
| ATP7B | Wilson's disease |
| ATXN1 | Spinocerebellar ataxia |
| ATXN2 | Amyotrophic lateral sclerosis |
| ATXN3 | Machado-Joseph disease |
| ATXN7 | Ataxia |
| Autotaxin | Idiopathic pulmonary fibrosis |
| IL-12 | Melanoma |
| IL-12 | Lung cancer |
| IL-12 | HPV-associated cancer |
| IL-12, | Advanced malignant solid |
| IL-15 | tumor |
| IL-12, | Solid tumor |
| IL-15, | |
| PDL1 | |
| IL-12, IL-2 | Melanoma |
| IL-12, | Ovarian cancer |
| MUC16 | |
| IL-12, PD-1 | Glioblastoma |
| IL-12, PD-1 | Glioma |
| IL-12, PD-1 | Recurrent glioblastoma |
| IL-12, PD-1 | Glioblastoma multiforme |
| IL-12, | Astrocytoma |
| thymidine | |
| kinase | |
| IL-12, | Recurrent prostate cancer |
| thymidine | |
| kinase | |
| IL-12R | Metastatic breast cancer |
| IL-12R | Metastatic colorectal cancer |
| IL-12R | Primary peritoneal |
| | carcinoma |
| IL-12R | Pediatric cerebellar |
| | astrocytoma |
| IL-12R | Advanced malignant solid |
| | tumor |
| IL-12R | Head and neck tumor |
| IL-12R | Fallopian tube cancer |
| IL-12R | Gliosarcoma |
| IL-12R | Mucolipidosis type II |
| AXL | Sarcoma |
| AXL | Ovarian cancer |
| AXL | Osteosarcoma |
| B2M, HLA-B | Head and neck tumor |
| B2M, HLA-B | Renal tumor |
| B2M, HLA-B | Breast cancer |
| B2M, HLA-B | Lymphoma |
| B2M, HLA-B | Colorectal cancer |
| B2M, HLA-B | Melanoma |
| B4GALT1 | Cardiovascular disease |
| B7-H4 | Solid tumor |
| BAFF | Autoimmune disease |
| BAFF | Systemic lupus |
| | erythematosus |
| BAFF | Refractory non-Hodgkin |
| | lymphoma |
| BAFF | Refractory plasma cell |
| | myeloma |
| BAFF | Sjögren's syndrome |
| BAFF | Relapsed non-Hodgkin |
| | lymphoma |
| BAFF | Relapsed Multiple myeloma |
| BAFF | B-cell lymphoma |
| BAFF | B-cell malignancy |
| BAFF, CD19 | Autoimmune disease |
| BAFF-R | Refractory transformed |
| | chronic lymphocytic |
| | leukemia |
| BAFF-R | Refractory small lymphocytic |
| | lymphoma |
| BAFF-R | Refractory mantle cell |
| | lymphoma |
| BAFF-R | Refractory diffuse large |
| | B-cell lymphoma |
| BAFF-R | Refractory chronic |
| | lymphocytic leukemia |
| IL-12R | Diffuse intrinsic pontine |
| | glioma |
| IL-12R | Ovarian epithelial |
| | carcinoma |
| IL-12R | Ovarian cancer |
| IL-12R | Locally advanced breast |
| | cancer |
| IL-12R | Colorectal cancer with liver |
| | metastasis |
| IL-12R | Colorectal cancer |
| IL-12R | Glioblastoma |
| IL-12R | Glioma |
| IL-12R | Melanoma |
| IL-12R | Secondary malignant |
| | neoplasm of peritoneum |
| IL-12R | Peritoneal cancer |
| IL-12R | Recurrent breast cancer |
| IL-12R | Recurrent glioblastoma |
| IL-12R | Recurrent malignant glioma |
| IL-12R | Glioblastoma multiforme |
| IL-12R | Metabolic bone disease |
| IL-12R | Unresectable melanoma |
| IL-12R | WHO grade III mixed |
| | glioma |
| IL-12R, | Gastrointestinal tumor |
| decorin | |
| IL-12R, | Myeloproliferative disorder |
| decorin | |
| IL-12R, | Multiple myeloma |
| decorin | |
| IL-12R, | Solid tumor |
| IL 15R | |
| IL-12R, | Renal cell carcinoma |
| IL15R | |
| IL-12R, | Breast cancer |
| IL15R | |
| IL-12R, | Ovarian cancer |
| IL15R | |
| IL-12R, | Hematologic malignancy |
| IL-15Rα | |
| BAFF-R | Refractory follicular |
| | lymphoma |
| BAFF-R | Diffuse large B-cell |
| | lymphoma |
| BAFF-R | Relapsed transformed chronic |
| | lymphocytic leukemia |
| BAFF-R | Relapsed mantle cell |
| | lymphoma |
| BAFF-R | Relapsed chronic |
| | lymphocytic leukemia |
| BAFF-R | Relapsed follicular |
| | lymphoma |
| BAFF-R | Relapsed marginal zone |
| | lymphoma |
| BAFF-R | Refractory marginal zone |
| | lymphoma |
| BAFF-R | B-cell malignancy |
| BAG3 | Dilated cardiomyopathy |
| BCL11A | Sickle cell disease |
| BCL11A | β-thalassemia |
| BCL11A, | Sickle cell disease |
| Hemoglobins | |
| BCL11A, | Transfusion-dependent |
| β-globin | β-thalassemia |
| BCL11A, | Sickle cell disease |
| β-globin | |
| BCL11A, | β-thalassemia |
| β-globin | |
| Bcl-2 | Metastatic renal cell |
| | carcinoma |
| IL-12R, | Solid tumor |
| IL-15Rα | |
| IL-12R, | Metastatic gastric |
| IL-15Rα, | adenocarcinoma |
| PDL1 | |
| IL-12R, | Metastatic gastroesophageal |
| IL-15Rα, | adenocarcinoma |
| PDL1 | |
| IL-12R, | Metastatic gastroesophageal |
| IL-15Rα, | junction adenocarcinoma |
| PDL1 | |
| IL-12R, | Advanced pancreatic |
| IL-15Rα, | adenocarcinoma |
| PDL1 | |
| IL-12R, | Advanced hepatocellular |
| IL-15Rα, | carcinoma |
| PDL1 | |
| IL-12R, | Advanced malignant solid |
| IL-15Rα, | tumor |
| PDL1 | |
| IL-12R, | Osteosarcoma |
| IL-15Rα, | |
| PDL1 | |
| IL-12R, | Hepatocellular carcinoma |
| IL-15Rα, | |
| PDL1 | |
| IL-12R, | Intrahepatic |
| IL-15Rα, | cholangiocarcinoma |
| PDL1 | |
| IL-12R, | Liver cancer |
| IL-15Rα, | |
| PDL1 | |
| IL-12R, | Cholangiocarcinoma |
| IL-15Rα, | |
| PDL1 | |
| IL-12R, | Metastatic solid tumor |
| IL-7Rα | |
| IL-12R, | Advanced cancer |
| IL-7Rα | |
| IL-12R, | Tumor |
| NY-ESO-1 | |
| IL-12R, | Endometrial cancer |
| PD-1 | |
| IL-12R, | Metastatic colorectal cancer |
| PD-1 | |
| Bcl-2 | Metastatic breast cancer |
| Bcl-2 | Intraocular lymphoma |
| Bcl-2 | Recurrent small cell lung |
| | cancer |
| Bcl-2 | Small lymphocytic |
| | lymphoma |
| Bcl-2 | Small intestine cancer |
| Bcl-2 | Gastrointestinal stromal |
| | tumor |
| Bcl-2 | Advanced malignant solid |
| | tumor |
| Bcl-2 | Peripheral T-cell lymphoma |
| Bcl-2 | Mantle cell lymphoma |
| Bcl-2 | Breast cancer |
| Bcl-2 | Prostate cancer |
| Bcl-2 | Cutaneous T-cell lymphoma |
| Bcl-2 | Refractory acute myeloid |
| | leukemia |
| Bcl-2 | Refractory non-Hodgkin |
| | lymphoma |
| Bcl-2 | Male breast tumor |
| Bcl-2 | Immunoblastic large cell |
| | lymphoma |
| Bcl-2 | Diffuse large B-cell |
| | lymphoma |
| Bcl-2 | Merkel cell carcinoma |
| Bcl-2 | Chronic lymphocytic |
| | leukemia |
| Bcl-2 | Follicular lymphoma |
| Bcl-2 | Lymphocytic leukemia |
| Bcl-2 | Lymphoblastic lymphoma |
| Bcl-2 | Reye's syndrome |
| Bcl-2 | Macroglobulinemia |
| IL-12R, | Advanced malignant solid |
| PD-1 | tumor |
| IL-12R, | Head and neck squamous |
| PD-1 | cell carcinoma |
| IL-12R, | Esophageal cancer |
| PD-1 | |
| IL-12R, | Breast cancer |
| PD-1 | |
| IL-12R, | Sarcoma |
| PD-1 | |
| IL-12R, | Skin tumor |
| PD-1 | |
| IL-12R, | Ovarian cancer |
| PD-1 | |
| IL-12R, | Lymphoma |
| PD-1 | |
| IL-12R, | Colorectal cancer with liver |
| PD-1 | metastasis |
| IL-12R, | Colorectal cancer |
| PD-1 | |
| IL-12R, | Melanoma |
| PD-1 | |
| IL-12R, | Liver cancer |
| PD-1 | |
| IL-12R, | Non-small cell lung cancer |
| PD-1 | |
| IL-12R, | Malignant pleural |
| PD-1 | mesothelioma |
| IL-12R, | Bladder cancer |
| PD-1 | |
| IL-12R, | Non-muscle-invasive |
| RIG-I | bladder tumor |
| IL-12R, | Prostate cancer |
| STEAP1 | |
| IL-13R, | Asthma |
| IL-4Rα | |
| IL-13R, | Allergic asthma |
| IL-4Rα | |
| IL-13R, | Rhinitis |
| IL-4Rα | |
| IL-13Rα2 | Brain metastases |
| IL-13Rα2 | Glioblastoma |
| IL-13Rα2 | Glioma |
| IL-13Rα2 | Melanoma |
| Bcl-2 | Locally advanced breast |
| | cancer |
| Bcl-2 | Glioblastoma |
| Bcl-2 | Plasmacytoma |
| Bcl-2 | Acute myeloid leukemia |
| Bcl-2 | Acute lymphoblastic |
| | leukemia |
| Bcl-2 | Hodgkin lymphoma |
| Bcl-2 | Refractory Waldenstrom's |
| | macroglobulinemia |
| Bcl-2 | Melanoma |
| Bcl-2 | Extensive-stage small cell |
| | lung cancer |
| Bcl-2 | Testicular disorders |
| Bcl-2 | Liver cancer |
| Bcl-2 | Recurrent mantle cell |
| | lymphoma |
| Bcl-2 | Recurrent diffuse large B-cell |
| | lymphoma |
| Bcl-2 | Recurrent acute myeloid |
| | leukemia |
| Bcl-2 | Recurrent Hodgkin |
| | lymphoma |
| Bcl-2 | Recurrent non-Hodgkin |
| | lymphoma |
| Bcl-2 | Recurrent adult grade III |
| | lymphogranuloma |
| Bcl-2 | Relapsed marginal zone |
| | lymphoma |
| Bcl-2 | Relapsed B-cell lymphoma |
| Bcl-2 | Recurrent Grade 3a Follicular |
| | Lymphoma |
| Bcl-2 | Lung cancer |
| Bcl-2 | Non-small cell lung cancer |
| Bcl-2 | Non-Hodgkin lymphoma |
| Bcl-2 | Multiple myeloma |
| Bcl-2 | Adult T-cell |
| | leukemia/lymphoma |
| IL-13Rα2 | High-grade astrocytoma |
| IL-13Rα2 | Recurrent glioblastoma |
| IL-13Rα2 | Recurrent malignant glioma |
| IL-13Rα2 | Pleomorphic glioblastoma |
| IL-15 | Metastatic melanoma |
| IL-15 | Metastatic non-small cell |
| | lung cancer |
| IL-15 | Tumor |
| IL-15 | Gastric cancer |
| IL-15 | Solid tumor |
| IL-15 | Lymphoma |
| IL-15 | Colorectal cancer |
| IL-15 | Secondary malignant lung |
| | tumor |
| IL-15 | Melanoma |
| IL-15 | Non-small cell lung cancer |
| IL-15, IL-2 | Tumor |
| IL-15, | Non-small cell lung cancer |
| PDL1 | |
| IL-15, | Pancreatic acinar cell |
| PSCA | carcinoma |
| IL-15, | Gastric cancer |
| PSCA | |
| IL-15, | Prostate cancer |
| PSCA | |
| IL-15, | Bladder cancer |
| PSCA | |
| IL15R, | Fallopian tube cancer |
| MUC16 | |
| IL15R, | Refractory ovarian cancer |
| MUC16 | |
| IL15R, | Ovarian cancer |
| MUC16 | |
| IL15R, | Peritoneal cancer |
| MUC16 | |
| IL15R, | Recurrent primary |
| MUC16 | peritoneal cancer |
| Bcl-2 | Burkitt lymphoma |
| Bcl-2 | Marginal zone B-cell |
| | lymphoma |
| Bcl-2 | Triple-Negative Breast |
| | Cancer |
| Bcl-2 | Type 1 diabetes |
| Bcl-2, c-Myc | Ovarian cancer |
| Bcl-2, c-Myc | Lung cancer |
| Bcl-xl | Tumor |
| Bcl-xl | Colorectal cancer |
| Bcl-xl, Mcl-1 | Head and neck tumor |
| Bcl-xl, Mcl-1 | Bladder cancer |
| BCMA | Autoimmune disease |
| BCMA | Myasthenia gravis |
| BCMA | End-stage renal disease |
| BCMA | Smoldering Multiple |
| | myeloma |
| BCMA | Systemic lupus |
| | erythematosus |
| BCMA | Peripheral T-cell lymphoma |
| BCMA | Aquaporin 4 |
| | antibody-positive |
| | neuromyelitis optica |
| | spectrum disorder |
| BCMA | Neuromyelitis optica |
| BCMA | Precursor T-cell acute |
| | lymphoblastic |
| | leukemia/lymphoma |
| BCMA | Immunoblastic |
| | lymphadenopathy |
| BCMA | Chronic inflammatory |
| | demyelinating |
| | polyneuropathy |
| BCMA | Extranodal NK-T cell |
| | lymphoma |
| BCMA | Refractory Multiple myeloma |
| IL15R, | Recurrent ovarian cancer |
| MUC16 | |
| IL15R, | Recurrent platinum-resistant |
| MUC16 | primary peritoneal cancer |
| IL15R, | Platinum-resistant fallopian |
| MUC16 | tube cancer |
| IL15R, | Platinum-resistant ovarian |
| MUC16 | cancer |
| IL15R, | Lung cancer |
| PD-1 | |
| IL15R, | Non-small cell lung cancer |
| PD-1 | |
| IL-15Rα, | Metastatic colorectal cancer |
| NKG2D | |
| IL-15Rα, | Osteosarcoma |
| NKG2D | |
| IL-15Rα, | Hepatocellular carcinoma |
| NKG2D | |
| IL-15Rα, | Intrahepatic |
| NKG2D | cholangiocarcinoma |
| IL-17RA | Psoriasis |
| IL-17RA | Congenital ichthyosis |
| | erythroderma |
| IL-17RA | Hair loss |
| IL-18 | Hematologic malignancy |
| IL-18 | Solid tumor |
| IL-18 | Renal cell carcinoma |
| IL-18 | Neuralgia |
| IL-1R, | Liver cirrhosis |
| MMPs | |
| IL1R1 | Gout |
| IL1R1 | Arthritis |
| IL-1RA | Knee arthritis |
| IL-1RA, | Osteoarthritis |
| PRG4 | |
| IL-1α | Osteoarthritis |
| BCMA | Plasma cell leukemia |
| BCMA | Anaplastic large cell |
| | lymphoma |
| BCMA | Necrotizing myopathy |
| BCMA | Multiple myeloma |
| BCMA | Enteropathy-associated T-cell |
| | lymphoma |
| BCMA | CD7-positive hematological |
| | malignancy |
| BCMA, | Multiple myeloma |
| CD138, | |
| CD19, CD38 | |
| BCMA, | Refractory Multiple myeloma |
| CD16a, | |
| IL-15Rα | |
| BCMA, | Relapsed Multiple myeloma |
| CD16a, | |
| IL-15Rα | |
| BCMA, | Immunoglobulin light chain |
| CD16a, | amyloidosis |
| NKp46 | |
| BCMA, | Refractory Multiple myeloma |
| CD16a, | |
| NKp46 | |
| BCMA, | Myelodysplastic syndrome |
| CD16a, | |
| NKp46 | |
| BCMA, | Relapsed Multiple myeloma |
| CD16a, | |
| NKp46 | |
| BCMA, | Autoimmune hemolytic |
| CD19 | anemia |
| BCMA, | Vasculitis |
| CD19 | |
| BCMA, | Systemic lupus |
| CD19 | erythematosus |
| BCMA, | Precursor B-cell |
| CD19 | lymphoblastic |
| | leukemia/lymphoma |
| BCMA, | Precursor/B-cell |
| CD19 | lymphoblastic leukemia |
| | lymphoma |
| BCMA, | Sjögren's syndrome |
| CD19 | |
| IL-2 | Primary peritoneal |
| | adenocarcinoma |
| IL-2 | Primary peritoneal cancer |
| IL-2 | Adenocarcinoma |
| IL-2 | Head and neck tumor |
| IL-2 | Fallopian tube cancer |
| IL-2 | Renal cell carcinoma |
| IL-2 | Neuroblastoma |
| IL-2 | Breast cancer |
| IL-2 | Prostate cancer |
| IL-2 | Skin tumor |
| IL-2 | Ovarian adenocarcinoma |
| IL-2 | Ovarian serous |
| | adenocarcinoma |
| IL-2 | Colon cancer |
| IL-2 | Serous cystadenocarcinoma |
| IL-2 | Mesothelioma |
| IL-2 | Melanoma |
| IL-2 | Melanoma |
| IL-2 | Hepatocellular carcinoma |
| IL-2 | Lung cancer |
| BCMA, | Non-Hodgkin lymphoma |
| CD19 | |
| BCMA, | Multiple myeloma |
| CD19 | |
| BCMA, | Multiple myeloma |
| CD19 | |
| BCMA, | Amyloidosis |
| CD19 | |
| BCMA, | Relapsed T-cell acute |
| CD19 | lymphoblastic leukemia |
| BCMA, | POEMS syndrome |
| CD19 | |
| BCMA, | Crouzon syndrome with |
| CD19 | acanthosis nigricans |
| BCMA, | B-cell lymphoma |
| CD19 | |
| BCMA, | Tumor |
| CD19, HER2, | |
| Trop-2 | |
| BCMA, | Autoimmune disease |
| CD20 | |
| BCMA, CD3 | Tumor |
| BCMA, | Tumor |
| CD38 | |
| BCMA, CD4 | Tumor |
| BCMA, CD5 | Tumor |
| BCMA, CD7 | Multiple myeloma |
| BCMA, | Renal cell carcinoma |
| CD70 | |
| BCMA, | Refractory plasma cell |
| CD70 | myeloma |
| BCMA, | Multiple myeloma |
| CD70 | |
| BCMA, CD8 | Tumor |
| BCMA, | Multiple myeloma |
| GPRC5D | |
| BCMA, | Myeloid leukemia |
| GPRC5D, | |
| NKG2D | |
| BCMA, | Myeloproliferative disorder |
| HPK1 | |
| BCMA, | Multiple myeloma |
| SLAMF7 | |
| IL-2, TNF | Renal cell carcinoma |
| IL-2, TNF | Colon cancer |
| IL-2, TNF | Melanoma |
| IL-2, | Metastatic melanoma |
| TNF-α | |
| IL-2, | Head and neck squamous |
| TNF-α | cell carcinoma |
| IL-2, | Solid tumor |
| TNF-α | |
| IL-2, | Ovarian cancer |
| TNF-α | |
| IL-2, | Melanoma |
| TNF-α | |
| IL-2, | Non-small cell lung cancer |
| TNF-α | |
| IL-21 | Pancreatic cancer |
| IL-21 | Pulmonary arterial |
| | hypertension |
| IL-21R | Poxvirus infection |
| IL-23 | Tumor |
| IL-24 | Pancreatic cancer |
| IL-24 | Head and neck tumor |
| IL-24 | Breast cancer |
| IL-24 | Melanoma |
| IL-24 | Hepatocellular carcinoma |
| IL-24 | Lung cancer |
| IL-24, | Leukemia |
| THBS1 | |
| IL-24, | Hepatocellular carcinoma |
| TRAIL | |
| IL-27 | Tumor |
| IL-2R | Metastatic melanoma |
| BCMA, | Refractory plasma cell |
| TACI | myeloma |
| BCMA, | Relapsed Multiple myeloma |
| TACI | |
| BCMA, | Refractory B-cell acute |
| TGF-β | lymphoblastic leukemia |
| BCMA, | Multiple myeloma |
| TGF-β | |
| BCMA, | Relapsed T-cell acute |
| TGF-β | lymphoblastic leukemia |
| BCMA, | Multiple myeloma |
| VISTA | |
| BDNF | Glaucoma |
| BDNF | Alzheimer's disease |
| beclin-1 | Multiple myeloma |
| BEST1 | Vitelliform macular |
| | dystrophy |
| BET, RPE65 | Biallelic RPE65 |
| | Mutation-associated Retinal |
| | Degeneration |
| bFGF | Perioperative ischemia |
| bFGF | Achondroplasia |
| BMP4 | Glioblastoma |
| BMP6 | Sjögren's syndrome |
| BMPR2, | Fracture |
| VEGF | |
| BRAF | Tumor |
| BRCA1 | Ovarian cancer |
| BRD4 | Solid tumor |
| BSG | Liver cancer |
| BSG | Recurrent glioblastoma |
| BSG | Recurrent malignant glioma |
| BSG, IL-24 | Hepatocellular carcinoma |
| BTK | Bruton's |
| | agammaglobulinemia |
| BTN3A1 | Acute myeloid leukemia |
| BTN3A1 | Myelodysplastic syndrome |
| BTN3A1 | Multiple myeloma |
| IL-2R | Gastrointestinal tumor |
| IL-2R | Renal tumor |
| IL-2R | Renal tumor |
| IL-2R | Renal cell carcinoma |
| IL-2R | Prostate cancer |
| IL-2R | Mesothelioma |
| IL-2R | Melanoma |
| IL-2R | Recurrent prostate cancer |
| IL-2R | Lung cancer |
| IL-2R, | Cancer |
| LMP2 | |
| IL2RA | Tumor |
| IL2RA | Hematologic malignancy |
| IL-2Rγ | Immunodeficiency |
| | syndrome |
| IL-2Rγ | X-linked severe combined |
| | immunodeficiency |
| IL-3 | CD123-positive acute |
| | myeloid leukemia |
| IL-33R | Asthma |
| IL-4Rα | Asthma |
| IL-6R | Inflammation |
| IL-6R | Arthritis |
| IL-6RA | Rheumatoid arthritis |
| IL-7 | Tumor |
| IL-7 | Melanoma |
| IL-7Rα | Head and neck tumor |
| IL-7Rα | Cervical cancer |
| IL-8 | Pathological |
| | neovascularization |
| influenza | Influenza virus infection |
| virus M1 | |
| influenza | Influenza A virus infection |
| virus M1 | |
| C1-INH | Hereditary angioedema |
| C1-INH | Liver disease |
| C3 | Paroxysmal nocturnal |
| | hemoglobinuria |
| C3 | Neurodegenerative disease |
| C3 | Immune system disease |
| C3 | Immunoglobulin A |
| | nephropathy |
| C3 | Liver disease |
| C3 | Complement dysregulation |
| C3 | C3 glomerulopathy |
| C3, C5 | Geographic atrophy |
| C5 | Myasthenia gravis |
| C5 | Paroxysmal nocturnal |
| | hemoglobinuria |
| C5 | Stargardt disease |
| C5 | Age-related macular |
| | degeneration |
| C5 | Immune system disease |
| C5 | Immunoglobulin A |
| | nephropathy |
| C5 | Macular degeneration |
| C5 | Geographic atrophy |
| C5, C5AR1 | Sepsis |
| C5, CFB | Genetic disorder |
| C5a | Community-acquired |
| | pneumonia |
| C5a | Lung cancer |
| C9orf72 | Pick's disease dementia |
| C9orf72 | Amyotrophic lateral sclerosis |
| C9orf72 | Frontotemporal dementia |
| CA4 | Ocular hypertension |
| CA8 | Knee arthritis |
| CA8 | Erythromelalgia |
| CADM1 | Hepatocellular carcinoma |
| CADM1 | Non-small cell lung cancer |
| CAG repeat | Machado-Joseph disease |
| expansion | |
| CAG repeat | Spinocerebellar ataxia |
| expansion | |
| ING4 | Relapsed acute myeloid |
| | leukemia |
| INHBE | Abdominal obesity |
| INSR | Diabetic macular edema |
| INSR | Diabetes |
| INSR | Wet age-related macular |
| | degeneration |
| INSR | Ischemic central retinal vein |
| | occlusion |
| INSR | Choroidal |
| | neovascularization |
| INSR | Corneal transplant rejection |
| INSR | Macular degeneration |
| INSR | Bladder cancer |
| INSR | Type 1 diabetes |
| Insulin | Type 1 diabetes |
| Integrin | Tumor |
| Integrin | Prostate adenocarcinoma |
| Integrin | Localized prostate cancer |
| Ion | Idiopathic pulmonary |
| channels | fibrosis |
| Ion | Trigeminal neuralgia |
| channels | |
| Ion | Partial seizures |
| channels | |
| IRAK1 | Hepatocellular carcinoma |
| IRAK1 | Trauma and injury |
| IRF4 | Refractory plasmacytoma |
| IRF4 | Recurrent multiple myeloma |
| IRF5 | Rheumatoid arthritis |
| ISG15 | Hepatitis C |
| ITGB7 | Multiple myeloma |
| JAK1 | Autoimmune disease |
| JAK1 | Inflammation |
| KHK | Obesity |
| KHK | Type 2 diabetes |
| Kir7.1 | Leber congenital amaurosis |
| KKLC1 | Pancreatic acinar cell |
| | carcinoma |
| KKLC1 | Gastric cancer |
| CAG repeat | Hodgkin lymphoma |
| expansion | |
| CAG repeat | Huntington's disease |
| expansion | |
| CAIX | Advanced renal cell |
| | carcinoma |
| CAIX | Solid tumor |
| CAIX, PDL1 | Tumor |
| CAIX, | Renal cell carcinoma |
| PSMA | |
| CAIX, | Renal clear cell carcinoma |
| PSMA | |
| CaN | Liver transplant rejection |
| CAPN2 | Amyotrophic lateral sclerosis |
| CAPN3 | Limb-girdle muscular |
| | dystrophy |
| CAPN3 | Limb-girdle muscular |
| | dystrophy type 2A |
| Caspase 2 | Optic nerve injury |
| Caspase 2 | Ischemic optic neuropathy |
| Caspase 2 | Glaucoma |
| Caspase 2 | Angle-closure glaucoma |
| CASQ2 | Ventricular tachycardia |
| CAV1 | Amyotrophic lateral sclerosis |
| Caveolin | Wet age-related macular |
| | degeneration |
| Cbl-b | Endometrial cancer |
| Cbl-b | Pancreatic cancer |
| Cbl-b | Advanced cancer |
| Cbl-b | Renal cell carcinoma |
| Cbl-b | Glioblastoma multiforme |
| Cbl-b | Cervical cancer |
| Cbl-b | Recurrent melanoma |
| Cbl-b | Platinum-resistant ovarian |
| | cancer |
| KKLC1 | Breast cancer |
| KKLC1 | Cervical cancer |
| KKLC1 | Liver cancer |
| KKLC1 | Lung cancer |
| KKLC1 | Non-small cell lung cancer |
| KLB | Alzheimer's disease |
| KLK2 | Metastatic |
| | castration-resistant prostate |
| | cancer |
| KLK3 | Prostate cancer |
| KLK5 | Netherton syndrome |
| KLKB1 | Hereditary angioedema |
| KMA | Multiple myeloma |
| KRAS | Tumor |
| KRAS | Pancreatic cancer |
| KRAS | Immune system disease |
| KRAS | Colorectal cancer |
| KRAS | Muscle disorders |
| KRAS | Lung cancer |
| KRAS | Non-small cell lung cancer |
| KRAS | KRAS-mutant tumor |
| KRAS | Lung cancer |
| G12A | |
| KRAS | Tumor |
| G12C | |
| KRAS | Lung cancer |
| G12C | |
| KRAS | Non-small cell lung cancer |
| G12C | |
| KRAS | Pancreatic ductal |
| G12C, | adenocarcinoma |
| KRAS | |
| G12D | |
| KRAS | Colorectal cancer |
| G12C, | |
| KRAS | |
| G12D | |
| KRAS | Tumor |
| G12C, | |
| KRAS | |
| G12D, | |
| KRAS | |
| G12V | |
| Cbl-b | Stage IIB melanoma |
| cccDNA | Hepatitis B |
| CCL19, | Diffuse large B-cell |
| CD19, | lymphoma |
| IL-7Rα | |
| CCL19, IL-7, | Solid tumor |
| MAGEA4 | |
| CCL2 | Diabetic nephropathy |
| CCL21 | Lung cancer |
| CCL4, | Advanced malignant solid |
| CTLA4, | tumor |
| Flt3L, IL-12, | |
| PD-1 | |
| CCL4, | Head and neck squamous cell |
| CTLA4, | carcinoma |
| Flt3L, IL-12, | |
| PD-1 | |
| CCL4, | Triple-negative breast cancer |
| CTLA4, | |
| Flt3L, IL-12, | |
| PD-1 | |
| CCL4, | Colorectal cancer |
| CTLA4, | |
| Flt3L, IL-12, | |
| PD-1 | |
| KRAS | Pancreatic cancer |
| G12C, | |
| KRAS | |
| G12D, | |
| KRAS | |
| G12V | |
| KRAS | Colon cancer |
| G12C, | |
| KRAS | |
| G12D, | |
| KRAS | |
| G12V | |
| KRAS | Lung cancer |
| G12C, | |
| KRAS | |
| G12D, | |
| KRAS | |
| G12V | |
| KRAS | Tumor |
| G12C, | |
| KRAS | |
| G12D, | |
| PI3Kα, p53 | |
| R175H | |
| KRAS | Endometrial cancer |
| G12D | |
| KRAS | Metastatic pancreatic ductal |
| G12D | adenocarcinoma |
| KRAS | Rectal cancer |
| G12D | |
| KRAS | Pancreatic acinar cell |
| G12D | carcinoma |
| KRAS | Pancreatic ductal |
| G12D | adenocarcinoma |
| KRAS | Pancreatic cancer |
| G12D | |
| CCL4, | Melanoma |
| CTLA4, | |
| Flt3L, IL-12, | |
| PD-1 | |
| CCL4, | Liver metastasis |
| CTLA4, | |
| Flt3L, IL-12, | |
| PD-1 | |
| CCL4, | Liver cancer |
| CTLA4, | |
| Flt3L, IL-12, | |
| PD-1 | |
| CCL4, | Tumor |
| CXCL10, | |
| IL-12 | |
| CCL5 | Hepatocellular carcinoma |
| CCL5, CD19, | Solid tumor |
| IL-12, PD-1, | |
| Trop-2 | |
| CCNB1, | Breast cancer |
| WT1 | |
| CCND1, | Inflammatory bowel disease |
| ITGB7, | |
| MAdCAM-1, | |
| α4β7 | |
| CCND1, Raf | Tumor |
| kinase | |
| CCR1, EGFR | Glioblastoma |
| CCR2, IL-2, | Solid tumor |
| leptin | |
| CCR3, | Asthma |
| IL-3R, | |
| IL-3Rβ | |
| CCR3, | Allergic asthma |
| IL-3R, | |
| IL-3Rβ | |
| CCR5 | Breast cancer |
| CCR5 | Lymphoma |
| CCR5 | HIV infection |
| CCR5, CD4 | HIV infection |
| KRAS | Gastric cancer |
| G12D | |
| KRAS | Solid tumor |
| G12D | |
| KRAS | Locally advanced pancreatic |
| G12D | adenocarcinoma |
| KRAS | Colorectal cancer |
| G12D | |
| KRAS | Colon cancer |
| G12D | |
| KRAS | Lung cancer |
| G12D | |
| KRAS | Non-small cell lung cancer |
| G12D | |
| KRAS | Solid tumor |
| G12D, | |
| KRAS | |
| G12V | |
| KRAS | Metastatic pancreatic |
| G12V | adenocarcinoma |
| KRAS | Metastatic non-small cell |
| G12V | lung cancer |
| KRAS | Tumor |
| G12V | |
| KRAS | Pancreatic ductal |
| G12V | adenocarcinoma |
| KRAS | Pancreatic cancer |
| G12V | |
| KRAS | Solid tumor |
| G12V | |
| KRAS | Colorectal cancer |
| G12V | |
| KRAS | Lung cancer |
| G12V | |
| KRAS | Non-small cell lung cancer |
| G12V | |
| CCR5, | Pancytopenia |
| TRIM5 | |
| CCR5, | X-linked severe combined |
| TRIM5 | immunodeficiency |
| CD103, | Tumor |
| CD39, CD8 | |
| CD123 | Acute myeloid leukemia |
| CD123 | Acute lymphoblastic |
| | leukemia |
| CD123 | Myelodysplastic syndrome |
| CD123 | Blastic plasmacytoid |
| | dendritic cell neoplasm |
| CD123, | Tumor |
| CD33 | |
| CD123, | Tumor |
| CD33, CD38, | |
| CD56, | |
| CLL-1, | |
| MUC1 | |
| CD123, | Tumor |
| CD33, CLL-1 | |
| CD123, | Acute myeloid leukemia |
| TIM3 | |
| CD133 | Retinal disease |
| CD133 | Glioblastoma |
| CD133, | Glioblastoma |
| EGFR | |
| CD138 | Tumor |
| CD138, | Multiple myeloma |
| NY-ESO-1, | |
| SLAMF7, | |
| WT1 | |
| CD16a | Pancreatic cancer |
| CD16a | Novel Coronavirus Infection |
| CD16a | Advanced solid malignant |
| | tumor |
| CD16a | Fallopian tube cancer |
| CD16a | Breast cancer |
| KRAS | Colonic adenocarcinoma |
| G12V | |
| KRAS | Intestinal tumor |
| G12V | |
| KRAS | Pancreatic cancer |
| G13D | |
| KRAS, | Lung cancer |
| c-Myc | |
| KRT6A | Congenital pachyonychia |
| Ku70/80, | Metastatic solid tumor |
| MRN, | |
| PARP1 | |
| Ku70/80, | Breast cancer |
| MRN, | |
| PARP1 | |
| Ku70/80, | Prostate cancer |
| MRN, | |
| PARP1 | |
| Ku70/80, | Recurrent ovarian cancer |
| MRN, | |
| PARP1 | |
| Kv7.2 | epilepsy |
| L1CAM | Neuroendocrine prostate |
| | cancer |
| L1CAM | Neuroblastoma |
| L1CAM | leukemia |
| L1CAM | CD22-positive acute |
| | lymphoblastic leukemia |
| LICAM | CD19 expressing |
| | malignancy |
| LAG3 | Tumor |
| LAGE-1a, | Solid tumor |
| NY-ESO-1 | |
| LAMA1 | Muscular dystrophy |
| LAMP-1, | Acute myeloid leukemia |
| TERT | |
| LAMP-2 | Glycogen storage disease |
| | type IIb |
| LAMP-2 | Diverticular disease |
| CD16a | Hypoxia |
| CD16a | Ovarian epithelial carcinoma |
| CD16a | Colorectal cancer |
| CD16a | Acute myeloid leukemia |
| CD16a | Liver cancer |
| CD16a | Peritoneal cancer |
| CD16a | Lung cancer |
| CD16a | Glioblastoma multiforme |
| CD16a | B-cell lymphoma |
| CD16a, | Mantle cell lymphoma |
| CD19, | |
| IL-15Rα | |
| CD16a, | Diffuse large B-cell |
| CD19, | lymphoma |
| IL-15Rα | |
| CD16a, | Chronic lymphocytic |
| CD19, | leukemia |
| IL-15Rα | |
| CD16a, | Follicular lymphoma |
| CD19, | |
| IL-15Rα | |
| CD16a, | Indolent non-Hodgkin |
| CD19, | lymphoma |
| IL-15Rα | |
| CD16a, | Marginal zone B-cell |
| CD19, | lymphoma |
| IL-15Rα | |
| CD16a, | B-cell lymphoma |
| CD19, | |
| IL-15Rα | |
| CD16a, | Solid tumor |
| CD276, IL-7 | |
| CD16a, | HER2-positive breast cancer |
| HER2 | |
| CD16a, IL-15 | Advanced solid tumor |
| CD16a, IL-15 | Refractory plasma cell |
| | myeloma |
| CD16a, IL-15 | Acute myeloid leukemia |
| CD16a, IL-15 | Relapsed Multiple myeloma |
| CD16a, IL-15 | Multiple myeloma |
| LAMP-2 | Huntington's disease |
| LCA5 | Retinal degeneration |
| LCA5 | Leber congenital amaurosis |
| | type 5 |
| LCN2 | Pancreatic cancer |
| LDLR | Hypercholesterolemia |
| LECT2 | Amyloidosis |
| LEKTI | Netherton syndrome |
| lepB | Pseudomonas aeruginosa |
| | infection |
| Lewis-Y | Advanced cancer |
| antigen | |
| Lewis-Y | Acute myeloid leukemia |
| antigen | |
| LGR5 | Metastatic colorectal cancer |
| LGR5 | Hematologic malignancy |
| LGR5 | Ovarian cancer |
| LGR5 | Colorectal cancer |
| L-HBsAg | Hepatitis B |
| L-HBsAg | Fibrosis |
| L-HBsAg | Chronic hepatitis B |
| L-HBsAg | Chronic hepatitis D |
| LIGHT | Glioblastoma |
| LILRB4 | Refractory acute myeloid |
| | leukemia |
| LILRB4 | Chronic myelomonocytic |
| | leukemia |
| LILRB4 | Acute myeloid leukemia |
| LILRB4 | Acute myelomonocytic |
| | leukemia |
| CD16a, | Pancreatic cancer |
| IL-15, MICA, | |
| MICB | |
| CD16a, | Gastroesophageal junction |
| IL-15, MICA, | cancer |
| MICB | |
| CD16a, | Advanced solid tumor |
| IL-15, MICA, | |
| MICB | |
| CD16a, | Head and neck tumor |
| IL-15, MICA, | |
| MICB | |
| CD16a, | Breast cancer |
| IL-15, MICA, | |
| MICB | |
| CD16a, | Ovarian cancer |
| IL-15, MICA, | |
| MICB | |
| CD16a, | Colorectal cancer |
| IL-15, MICA, | |
| MICB | |
| CD16a, | Non-small cell lung cancer |
| IL-15, MICA, | |
| MICB | |
| CD18 | Leukocyte Adhesion |
| | Deficiency Type 1 |
| CD19 | Autoimmune disease |
| CD19 | Myasthenia gravis |
| CD19 | End-stage renal disease |
| CD19 | Primary progressive multiple |
| | sclerosis |
| CD19 | Graft-versus-host disease |
| CD19 | Pancreatic cancer |
| CD19 | Inflammation |
| CD19 | Small lymphocytic |
| | lymphoma |
| CD19 | Microscopic polyangiitis |
| CD19 | Systemic scleroderma |
| CD19 | Systemic lupus |
| | erythematosus |
| LILRB4 | Acute monocytic leukemia |
| LILRB4 | Relapsed acute myeloid |
| | leukemia |
| LILRB4 | Multiple myeloma |
| LIN28B | Pancreatic cancer |
| LIPG | Coronary heart disease |
| lipoprotein(a) | Aortic stenosis |
| lipoprotein(a) | Cardiovascular disease |
| lipoprotein(a) | Neurodegenerative disease |
| lipoprotein(a) | Hyperlipoproteinemia |
| lipoprotein(a) | Liver disease |
| lipoprotein(a) | Atherosclerosis |
| lipoprotein(a) | Hypobetalipoproteinemia |
| LIV-1 | Breast cancer |
| LMNA | Premature aging |
| LMNA | Myocardial disease |
| LMNA | Dilated cardiomyopathy |
| LMP1 | Hematologic malignancy |
| LMP1 | Nasopharyngeal carcinoma |
| LMP1, | Leiomyosarcoma |
| LMP2 | |
| LMP1, | Hodgkin lymphoma |
| LMP2 | |
| CD19 | Gastric cancer |
| CD19 | Idiopathic inflammatory |
| | myopathy |
| CD19 | Mantle cell lymphoma |
| CD19 | Granulomatosis with |
| | polyangiitis |
| CD19 | Precursor B-cell acute |
| | lymphoblastic leukemia |
| CD19 | Precursor B-cell |
| | lymphoblastic leukemia |
| | lymphoma |
| CD19 | Dermatomyositis |
| CD19 | Refractory acute leukemia |
| CD19 | Refractory non-Hodgkin |
| | lymphoma |
| CD19 | Refractory B-cell lymphoma |
| CD19 | Refractory B-type acute |
| | lymphoblastic leukemia |
| CD19 | Diffuse scleroderma |
| CD19 | Hairy cell leukemia |
| CD19 | Chronic lymphocytic |
| | leukemia |
| CD19 | Chronic myeloid leukemia |
| CD19 | Lymphomatoid |
| | granulomatosis |
| CD19 | Lymphoma |
| CD19 | Lupus nephritis |
| CD19 | Antineutrophil cytoplasmic |
| | antibody-associated vasculitis |
| CD19 | Secondary progressive |
| | multiple sclerosis |
| CD19 | Acute lymphoblastic |
| | leukemia |
| CD19 | Macular degeneration |
| CD19 | Philadelphia |
| | chromosome-negative acute |
| | lymphoblastic leukemia |
| LMP1, | Non-Hodgkin lymphoma |
| LMP2 | |
| LMP1, | Nasopharyngeal carcinoma |
| LMP2 | |
| LMP1, | Vaccination |
| MAVS | |
| LMP1, | HIV infection |
| MAVS | |
| LMP2 | Head and neck tumor |
| | metastasis |
| LMP2 | Recurrent nasopharyngeal |
| | carcinoma |
| LPL | Type V |
| | hyperlipoproteinemia |
| LPL | Hyperlipoproteinemia type I |
| LptD | Pseudomonas aeruginosa |
| | infection |
| LpxC | Pseudomonas aeruginosa |
| | infection |
| LRRC15 | Tumor |
| LRRK2 | Parkinson's disease |
| L-sel | Inflammation |
| LXR | Nonalcoholic steatohepatitis |
| LXR | Type II |
| | hyperlipoproteinemia |
| LY86 | Duchenne muscular |
| | dystrophy |
| LZTS1, | Tumor |
| LZTS2 | |
| MAFA, | Type 1 diabetes |
| PDX1 | |
| MAGEA1 | Tumor |
| MAGEA1 | Head and neck tumor |
| MAGEA1 | Solid tumor |
| MAGEA1 | Triple-negative breast |
| | cancer |
| MAGEA1 | Urothelial carcinoma |
| CD19 | Non-Hodgkin lymphoma |
| CD19 | Fanconi anemia |
| CD19 | Multiple sclerosis |
| CD19 | Residual tumor |
| CD19 | AIDS-related lymphoma |
| CD19 | CD19-positive B-cell acute |
| | lymphoblastic leukemia |
| CD19 | CD19-positive B-cell acute |
| | lymphoblastic leukemia |
| CD19 | B-cell lymphoma |
| CD19 | Type 2 diabetes |
| CD19, CD20 | Purpura hepatitis |
| CD19, CD20 | Precursor B-cell acute |
| | lymphoblastic leukemia |
| CD19, CD20 | Chronic lymphocytic |
| | leukemia |
| CD19, CD20 | Philadelphia |
| | chromosome-negative acute |
| | lymphoblastic leukemia |
| CD19, CD20, | Refractory non-Hodgkin |
| CD22 | lymphoma |
| CD19, CD20, | Refractory indolent |
| CD22 | non-Hodgkin lymphoma |
| CD19, CD20, | Chronic lymphocytic |
| CD22 | leukemia |
| CD19, CD20, | Acute lymphoblastic |
| CD22 | leukemia |
| CD19, CD20, | Relapsed transformed chronic |
| CD22 | lymphocytic leukemia |
| CD19, CD20, | Relapsed chronic |
| CD22 | lymphocytic leukemia |
| CD19, CD20, | Relapsed acute lymphoblastic |
| CD22 | leukemia |
| CD19, CD20, | Relapsed non-Hodgkin |
| CD22 | lymphoma |
| CD19, CD20, | Relapsed indolent |
| CD22 | non-Hodgkin lymphoma |
| CD19, CD20, | Relapsed B-type acute |
| CD22 | lymphoblastic leukemia |
| CD19, CD20, | B-cell lymphoma |
| CD22 | |
| CD19, CD22 | Autoimmune disease |
| CD19, CD22 | Purpura hepatitis |
| CD19, CD22 | Hematologic malignancy |
| MAGEA1 | Ovarian cancer |
| MAGEA1 | Melanoma |
| MAGEA1 | Melanoma |
| MAGEA1 | Cervical cancer |
| MAGEA1 | Hepatocellular carcinoma |
| MAGEA1 | Liver cancer |
| MAGEA1 | Non-small cell lung cancer |
| MAGEA1 | HPV-related cancers |
| MAGEA1, | Solid tumor |
| PRAME | |
| MAGEA10 | Head and neck tumor |
| MAGEA10 | Urothelial carcinoma |
| MAGEA10 | Melanoma |
| MAGEA10 | Non-small cell lung cancer |
| MAGEA10 | Malignant epithelial tumor |
| MAGEA12, | Metastatic melanoma |
| MAGEA3 | |
| MAGEA12, | Tumor metastasis |
| MAGEA3 | |
| MAGEA12, | Kidney tumor |
| MAGEA3 | |
| MAGEA3 | Kidney tumor |
| MAGEA3 | Breast cancer |
| MAGEA3 | Melanoma |
| MAGEA3 | Cervical cancer |
| MAGEA3 | Lung cancer |
| MAGEA3, | Tumor |
| MAGEA6 | |
| MAGEA3, | Solid tumor |
| MAGEA6 | |
| MAGEA4 | Endometrial cancer |
| MAGEA4 | Liposarcoma |
| MAGEA4 | Myxoid liposarcoma |
| CD19, CD22 | Blood disorder |
| CD19, CD22 | Precursor B-cell acute |
| | lymphoblastic leukemia |
| CD19, CD22 | Precursor B-cell |
| | lymphoblastic leukemia |
| | lymphoma |
| CD19, CD22 | Refractory B-type acute |
| | lymphoblastic leukemia |
| CD19, CD22 | Relapsed B-type acute |
| | lymphoblastic leukemia |
| CD19, CD22 | Philadelphia |
| | chromosome-negative acute |
| | lymphoblastic leukemia |
| CD19, CD22 | Philadelphia |
| | chromosome-positive acute |
| | lymphoblastic leukemia |
| CD19, CD22 | Non-Hodgkin lymphoma |
| CD19, CD22 | Residual tumor |
| CD19, CD22 | Ph-like acute lymphoblastic |
| | leukemia |
| CD19, CD22 | CD22-positive B-cell |
| | precursor acute |
| | lymphoblastic leukemia |
| CD19, CD22 | CD22-positive acute |
| | lymphoblastic leukemia |
| CD19, CD22 | CD22-positive B-cell acute |
| | lymphoblastic leukemia |
| CD19, CD22 | CD19-positive B-cell |
| | precursor acute |
| | lymphoblastic leukemia |
| CD19, CD22 | CD19-positive B-cell acute |
| | lymphoblastic leukemia |
| CD19, CD22 | B-cell lymphoma |
| CD19, CD22, | Chronic lymphocytic |
| CD8 | leukemia |
| CD19, CD22, | Acute lymphoblastic |
| CD8 | leukemia |
| CD19, CD22, | Non-Hodgkin lymphoma |
| CD8 | |
| CD19, | Ewing sarcoma |
| CD276 | |
| MAGEA4 | Gastroesophageal junction |
| | malignant tumor |
| MAGEA4 | Gastric cancer |
| MAGEA4 | Gastric cancer |
| MAGEA4 | Esophageal cancer |
| MAGEA4 | Solid tumor |
| MAGEA4 | Solid tumor |
| MAGEA4 | Neurofibrosarcoma |
| MAGEA4 | Neuroblastoma |
| MAGEA4 | sarcoma |
| MAGEA4 | Urothelial carcinoma |
| MAGEA4 | Ovarian cancer |
| MAGEA4 | Ovarian cancer |
| MAGEA4 | Synovial sarcoma |
| MAGEA4 | Melanoma |
| MAGEA4 | Osteosarcoma |
| MAGEA4 | Recurrent solid tumor |
| MAGEA4 | Non-small cell lung cancer |
| MAGEA4 | Bladder cancer |
| MAGEA4, | Solid tumor |
| MAGEA8 | |
| MAGEA4, | Hematologic malignancy |
| NY-ESO-1, | |
| PRAME, | |
| SSX2, | |
| survivin | |
| CD19, | Clear cell sarcoma |
| CD276 | |
| CD19, | Retinoblastoma |
| CD276 | |
| CD19, | Wilms tumor |
| CD276 | |
| CD19, | Neurofibrosarcoma |
| CD276 | |
| CD19, | Neuroblastoma |
| CD276 | |
| CD19, | sarcoma |
| CD276 | |
| CD19, | Synovial sarcoma |
| CD276 | |
| CD19, | Rhabdoid tumor |
| CD276 | |
| MAGEA4, | Acute myeloid leukemia |
| NY-ESO-1, | |
| PRAME, | |
| SSX2, | |
| survivin | |
| MAGEA4, | Hodgkin lymphoma |
| NY-ESO-1, | |
| PRAME, | |
| SSX2, | |
| survivin | |
| MAGEA4, | Non-Hodgkin lymphoma |
| NY-ESO-1, | |
| PRAME, | |
| SSX2, | |
| survivin | |
| MAGEA4, | Pancreatic cancer |
| NY-ESO-1, | |
| PRAME, | |
| SSX2, | |
| WT1, | |
| survivin | |
| MAGEA4, | Refractory lymphoma |
| NY-ESO-1, | |
| PRAME, | |
| SSX2, | |
| WT1, | |
| survivin | |
| MAGEA4, | Refractory non-Hodgkin |
| NY-ESO-1, | lymphoma |
| PRAME, | |
| SSX2, | |
| WT1, | |
| survivin | |
| MAGEA4, | Hodgkin lymphoma |
| NY-ESO-1, | |
| PRAME, | |
| SSX2, | |
| WT1, | |
| survivin | |
| MAGEA4, | Relapsed lymphoma |
| NY-ESO-1, | |
| PRAME, | |
| SSX2, | |
| WT1, | |
| survivin | |
| CD19, | Rhabdomyosarcoma |
| CD276 | |
| CD19, | Melanoma |
| CD276 | |
| CD19, | Hepatoblastoma |
| CD276 | |
| CD19, | Recurrent solid tumor |
| CD276 | |
| CD19, | Malignant epithelial tumor |
| CD276 | |
| CD19, | Desmoplastic small round |
| CD276 | cell tumor |
| CD19, CD3 | Severe combined |
| | immunodeficiency |
| CD19, CD3 | Common variable |
| | immunodeficiency |
| CD19, CD3 | Refractory B-cell lymphoma |
| CD19, CD3 | Immunodeficiency syndrome |
| CD19, CD3 | Mendelian susceptibility to |
| | mycobacterial disease |
| CD19, CD3 | Chronic granulomatous |
| | disease |
| CD19, CD3 | Lymphoma |
| CD19, CD3 | Relapsed B-cell lymphoma |
| CD19, CD3 | Wiskott-Aldrich syndrome |
| CD19, CD3 | Job syndrome |
| CD19, CD3 | B-cell lymphoma |
| CD19, CD4 | Chronic lymphocytic |
| | leukemia |
| CD19, CD4 | Non-Hodgkin lymphoma |
| MAGEA4, | Relapsed non-Hodgkin |
| NY-ESO-1, | lymphoma |
| PRAME, | |
| SSX2, | |
| WT1, | |
| survivin | |
| MAGEC2 | Head and neck tumor |
| MAGEC2 | Solid tumor |
| MAGEC2 | Ovarian cancer |
| MAGEC2 | Melanoma |
| MAGEC2 | Hepatocellular carcinoma |
| MAGEC2 | Non-small cell lung cancer |
| MALAT1 | Tumor |
| MALAT1 | Breast cancer |
| MAX, | Tumor |
| c-Myc | |
| MBP | Multiple sclerosis |
| MCM7 | Tumor |
| M-CSF | Tumor |
| MDA5, | Myxoid MFH |
| RIG-I, | |
| TLR3 | |
| MDA5, | Head and Neck Squamous |
| RIG-I, | Cell Carcinoma |
| TLR3 | |
| MDA5, | Neurofibrosarcoma |
| RIG-I, | |
| TLR3 | |
| MDA5, | Breast cancer |
| RIG-I, | |
| TLR3 | |
| MDA5, | Sarcoma |
| RIG-I, | |
| TLR3 | |
| MDA5, | Dedifferentiated |
| RIG-I, | liposarcoma |
| TLR3 | |
| CD19, CD7 | Precursor B-cell acute |
| | lymphoblastic leukemia |
| CD19, CD7 | Acute lymphoblastic |
| | leukemia |
| CD19, CD7 | B-cell leukemia |
| CD19, CD70 | Refractory B-cell lymphoma |
| CD19, CD70 | Lymphoma |
| CD19, CD70 | Acute myeloid leukemia |
| CD19, CD70 | Relapsed B-cell lymphoma |
| CD19, CD70 | Non-Hodgkin lymphoma |
| CD19, CD70 | B-cell lymphoma |
| CD19, | Non-Hodgkin lymphoma |
| CD79B | |
| CD19, CD8 | Precursor B-cell |
| | lymphoblastic leukemia |
| | lymphoma |
| CD19, CD8 | Chronic lymphocytic |
| | leukemia |
| CD19, CD8 | Non-Hodgkin lymphoma |
| CD19, | Gastric cancer |
| CLDN18.2 | |
| CD19, DR5 | B-cell lymphoma |
| CD19, EBV | Acute lymphoblastic |
| protein | leukemia |
| CD19, EBV | B-cell lymphoma |
| protein | |
| CD19, EGFR | Tumor |
| CD19, HPK1 | Acute lymphoblastic |
| | leukemia |
| CD19, HPK1 | B-cell lymphoma |
| MDA5, | Leiomyosarcoma |
| RIG-I, | |
| TLR3 | |
| MDA5, | Synovial sarcoma |
| RIG-I, | |
| TLR3 | |
| MDA5, | Rhabdomyosarcoma |
| RIG-I, | |
| TLR3 | |
| MDA5, | Hepatoembryosarcoma |
| RIG-I, | |
| TLR3 | |
| MDA5, | Retroperitoneal sarcoma |
| RIG-I, | |
| TLR3 | |
| MDA5, | Non-small cell lung cancer |
| RIG-I, | |
| TLR3 | |
| MDA5, | Malignant fibrous |
| RIG-I, | histiocytoma |
| TLR3 | |
| MDH1, | Non-small cell lung cancer |
| MDH2 | |
| MECOM | Ovarian cancer |
| MECOM | Lung cancer |
| MECP2 | Lubbs sex-linked mental |
| | retardation syndrome |
| MECP2 | Spinal muscular atrophy |
| | with respiratory distress |
| | type 1 |
| MECP2 | Charcot-Marie-Tooth |
| | Disease Type 2S |
| MECP2 | Rett syndrome |
| melan-A | Metastatic melanoma |
| melan-A | Melanoma |
| melan-A | Melanoma |
| MerTK | Retinitis pigmentosa |
| MEX3B | Severe asthma |
| MFN2 | Charcot-Marie-Tooth |
| | Disease |
| CD19, IL15R | Small lymphocytic |
| | lymphoma |
| CD19, IL15R | Mantle cell lymphoma |
| CD19, IL15R | Precursor B-cell acute |
| | lymphoblastic leukemia |
| CD19, IL15R | Chronic lymphocytic |
| | leukemia |
| CD19, IL15R | Lupus nephritis |
| CD19, IL15R | Macroglobulinemia |
| CD19, IL15R | Large B-cell lymphoma |
| CD19, IL15R | B-cell malignancy |
| CD19, IL-18 | Acute lymphoblastic |
| | leukemia |
| CD19, IL-18 | B-cell lymphoma |
| CD19, | Refractory B-type acute |
| IL18R1 | lymphoblastic leukemia |
| CD19, | Chronic lymphocytic |
| IL18R1 | leukemia |
| CD19, | Acute lymphoblastic |
| IL18R1 | leukemia |
| CD19, | Relapsed acute lymphoblastic |
| IL18R1 | leukemia |
| CD19, | Relapsed and refractory acute |
| IL18R1 | lymphoblastic leukemia |
| CD19, | Non-Hodgkin lymphoma |
| IL18R1 | |
| CD19, | CD19 expressing malignancy |
| IL18R1 | |
| CD19, | Tumor |
| IL-2Rβ | |
| CD19, | Small lymphocytic |
| IL-2Rβ | lymphoma |
| CD19, | Mantle cell lymphoma |
| IL-2Rβ | |
| CD19, | Diffuse large B-cell |
| IL-2Rβ | lymphoma |
| MFSD8 | Neuronal ceroid |
| | lipofuscinosis |
| MFSD8 | Lysosomal storage disease |
| MGMT | Glioblastoma |
| MICA | Hepatocellular carcinoma |
| MICA, | Tumor |
| MICB | |
| MICA, | Solid tumor |
| MICB, | |
| ULBP1 | |
| micro- | Duchenne muscular |
| dystrophin | dystrophy |
| microRNA | Hepatocellular carcinoma |
| let-7i-5p | |
| MicroRNAs | Alzheimer's disease |
| Microtubule- | Myocardial infarction |
| associated | |
| proteins | |
| Microtubule- | Nerve damage |
| associated | |
| proteins | |
| Microtubule- | burn |
| associated | |
| proteins | |
| miHAs | Hematopoietic stem cell |
| | transplantation |
| miHAs | Hematologic malignancy |
| miHAs | Hematologic malignancy |
| miHAs | Acute myeloid leukemia |
| miHAs | Acute lymphoblastic |
| | leukemia |
| miHAs | Myelodysplastic syndrome |
| miHAs | leukemia |
| mini- | Duchenne muscular |
| dystrophin | dystrophy |
| MIR 103A1, | Nonalcoholic steatohepatitis |
| miR-107 | |
| CD19, | Chronic lymphocytic |
| IL-2Rβ | leukemia |
| CD19, | Follicular lymphoma |
| IL-2Rβ | |
| CD19, | Indolent B-cell non-Hodgkin |
| IL-2Rβ | lymphoma |
| CD19, | Large B-cell lymphoma |
| IL-2Rβ | |
| CD19, | CD19 expressing malignancy |
| IL-2Rβ | |
| CD19, | Tumor |
| MUC1 | |
| CD19, PD-1 | Solid tumor |
| CD19, PD-1 | Refractory cancer |
| CD19, PD-1 | Refractory B-cell lymphoma |
| CD19, PD-1 | Relapsed non-Hodgkin |
| | lymphoma |
| CD19, PD-1 | Non-Hodgkin lymphoma |
| CD19, PD-1 | Mesothelin-positive tumor |
| CD19, PD-1 | CD19 expressing malignancy |
| CD19, PD-1 | Carney complex |
| CD19, PD-1 | B-cell lymphoma |
| CD19, PD-1, | Mediastinal large B-cell |
| TIGIT | lymphoma |
| CD19, PD-1, | Follicular lymphoma |
| TIGIT | |
| CD19, PD-1, | High-grade B-cell lymphoma |
| TIGIT | |
| CD19, PD-1, | Large B-cell lymphoma |
| TIGIT | |
| CD19, | Solid tumor |
| STING | |
| CD19, | Non-Hodgkin lymphoma |
| TGF-β2 | |
| CD19, | CD19-positive diffuse large |
| TGF-β2 | B-cell lymphoma |
| CD19, | CD19-positive B-cell acute |
| TGF-β2 | lymphoblastic leukemia |
| CD1A | Precursor T-lymphocytic |
| | leukemia lymphoma |
| CD1A | Precursor T-lymphocytic |
| | leukemia lymphoma |
| miR-10b | Pancreatic cancer |
| miR-10b | Small cell lung cancer |
| miR-10b | Advanced solid tumor |
| miR-10b | Breast cancer |
| miR-10b | Ovarian cancer |
| miR-10b | Colon cancer |
| miR-10b | Glioblastoma |
| miR-10b | Osteosarcoma |
| miR-10b, | Glioblastoma |
| miR-21 | |
| miR-122 | Hepatitis C |
| miR-126 | Chronic myeloid leukemia |
| miR-126 | Acute myeloid leukemia in |
| | adults |
| miR-132 | Heart failure |
| miR-132 | Cardiac hypertrophy |
| miR-132 | Heart failure with preserved |
| | ejection fraction |
| miR-132 | Dilated cardiomyopathy |
| miR-132 | Acute myocardial infarction |
| MIR135A1 | Depression |
| miR-143 | Colorectal cancer |
| miR-145 | Pulmonary hypertension |
| miR-150 | Rectal cancer |
| miR-150 | Colorectal cancer |
| miR-155 | Wet macular degeneration |
| miR-155 | Cutaneous T-cell lymphoma |
| miR-17 | Autosomal dominant |
| | polycystic kidney disease |
| CD1A | Lymphoma |
| CD1A | T-cell acute lymphoblastic |
| | leukemia/lymphoma |
| CD2 | Sezary syndrome |
| CD20 | Mediastinal large B-cell |
| | lymphoma |
| CD20 | Autoimmune disease |
| CD20 | Metastatic melanoma |
| CD20 | Hematologic malignancy |
| CD20 | Small lymphocytic |
| | lymphoma |
| CD20 | Skin melanoma |
| CD20 | Refractory mantle cell |
| | lymphoma |
| CD20 | Refractory B-cell lymphoma |
| CD20 | Chronic lymphocytic |
| | leukemia |
| CD20 | Follicular lymphoma |
| CD20 | Waldenstrom's |
| | macroglobulinemia is |
| | refractory |
| CD20 | Melanoma |
| CD20 | Relapsed mantle cell |
| | lymphoma |
| CD20 | Relapsed chronic |
| | lymphocytic leukemia |
| CD20 | Recurrent macroglobulinemia |
| CD20 | Relapsed B-cell lymphoma |
| CD20 | Marginal zone B-cell |
| | lymphoma |
| CD20 | CD20-positive B-cell |
| | lymphoma |
| CD20, CD22 | Precursor B-cell |
| | lymphoblastic leukemia |
| | lymphoma |
| CD20, CD22 | Refractory B-cell lymphoma |
| CD20, CD22 | Non-Hodgkin lymphoma |
| CD20, CD22, | Breast cancer |
| CD38 | |
| CD20, CD23, | Hematologic malignancy |
| TLR9 | |
| MIR181A2 | Chondrosarcoma |
| miR-193a-3p | Advanced solid tumor |
| miR-193a-3p | Melanoma |
| miR-193a-3p | Liver cancer |
| miR-195 | Liver cancer |
| miR-195 | Bile duct tumor |
| miR-21 | Post-COVID-19 Syndrome |
| miR-21 | Nephritis |
| miR-21 | Triple-negative breast |
| | cancer |
| miR-21 | Lung cancer |
| miR-21 | Non-small cell lung cancer |
| miR-21 | Bladder cancer |
| miR-21, | Triple-negative breast |
| miR-34a | cancer |
| miR-22 | Fatty liver |
| miR-22 | Optic nerve disease |
| miR-22 | Obesity |
| miR-22 | Nonalcoholic steatohepatitis |
| miR-22 | Metabolic disease |
| miR-22 | Metabolic fatty liver disease |
| miR-22 | Type 2 diabetes |
| miR-23b | Osteoarthritis |
| miR-29 | Fibrosis |
| miR-29 | Idiopathic pulmonary |
| | fibrosis |
| miR-328 | Shortsighted |
| miR-33a | Idiopathic pulmonary |
| | fibrosis |
| miR-34a | Small cell carcinoma |
| CD20, | Non-Hodgkin lymphoma |
| CD79A | |
| CD200R | Immune system disorder |
| CD22 | Autoimmune disease |
| CD22 | Hairy cell leukemia |
| CD22 | Acute lymphoblastic |
| | leukemia |
| CD22 | Non-Hodgkin lymphoma |
| CD22 | B-cell leukemia |
| CD22, CD37 | Hematologic malignancy |
| CD22, PDL1 | sarcoma |
| CD22, PDL1 | Cervical cancer |
| CD22, PDL1 | Non-small cell lung cancer |
| CD24 | Tumor |
| CD276 | Diffuse midline glioma with |
| | K27M point mutation in |
| | histone H3 |
| CD276 | Pancreatic ductal |
| | adenocarcinoma |
| CD276 | Pancreatic cancer |
| CD276 | Medulloblastoma |
| CD276 | Ependymoma |
| CD276 | Neuroblastoma |
| CD276 | Brain malignant glioma |
| CD276 | Refractory acute myeloid |
| | leukemia |
| CD276 | Diffuse intrinsic pontine |
| | glioma |
| CD276 | Ovarian epithelial carcinoma |
| CD276 | Colorectal cancer |
| CD276 | Colorectal cancer |
| CD276 | Glioblastoma |
| CD276 | Glioma |
| CD276 | Acute myeloid leukemia |
| CD276 | Melanoma |
| CD276 | Liver cancer |
| CD276 | Recurrent platinum-resistant |
| | ovarian cancer |
| miR-34a | Renal cell carcinoma |
| miR-34a | Lymphoma |
| miR-34a | Melanoma |
| miR-34a | Liver cancer |
| miR-34a | Non-small cell lung cancer |
| miR-34a | Multiple myeloma |
| MIR 449A | Breathing disorders |
| MIR92A1 | Heart failure |
| miR-96 | Diabetic nephropathy |
| MIRN122 | Parkinson's disease |
| microRNA | |
| MIRN122 | Amyotrophic lateral |
| microRNA | sclerosis |
| MIRN122 | Cholestasis |
| microRNA | |
| MIRN122 | Hepatitis C |
| microRNA | |
| MIRN122 | Alzheimer's disease |
| microRNA | |
| Mitochondrial | sarcoma |
| proteins | |
| Mitochondrial | Skin tumor |
| proteins | |
| Mitochondrial | Basal cell carcinoma |
| proteins | |
| Mitochondrial | Melanoma |
| proteins | |
| MKI67 | Bladder cancer |
| MLC | White matter disease |
| MMP1 | Tumor |
| MMP1 | Systemic scleroderma |
| MMP1 | Skin laxity |
| MMP1 | Facial wrinkle |
| MMP1 | aging |
| MMP1 | Trauma and Injury |
| MMP2 | Prostate cancer |
| MMP2 | Colorectal cancer |
| MMP2 | Glioblastoma |
| MMP2 | Melanoma |
| CD276 | Lung cancer |
| CD276 | Atypical teratoma |
| CD276 | Recurrent ovarian tumor of |
| | low malignant potential |
| CD276 | CD276-positive solid tumor |
| CD276, | Rhabdomyosarcoma |
| FGFR4 | |
| CD276, | Tumor |
| HER2 | |
| CD276, | Solid tumor |
| IL-13Rα2 | |
| CD28, CD80, | Tumor |
| p53 | |
| CD28, | Tumor |
| CSF-2R, | |
| CTLA4 | |
| CD29, | Metastatic non-small cell |
| EGFR, | lung cancer |
| LAMA4 | |
| CD29, | Breast cancer |
| EGFR, | |
| LAMA4 | |
| CD29, | Glioma |
| EGFR, | |
| LAMA4 | |
| CD3 | Tumor |
| CD3, CD7 | Acute lymphoblastic |
| | leukemia |
| CD3, CD80 | Tumor |
| CD3, CD86, | Endometrial cancer |
| PD-1 | |
| MMP2 | MMP2-positive |
| | glioblastoma |
| MMP-7 | Idiopathic pulmonary |
| | fibrosis |
| MNK1 | Non-small cell lung cancer |
| MnSOD | Colorectal cancer |
| MnSOD, | Non-small cell lung cancer |
| TRAIL | |
| MnSOD, | Motor neurone disease |
| transcription | |
| factors, | |
| and related | |
| regulatory | |
| factors | |
| MnSOD, | Traumatic brain injury |
| transcription | |
| factors, | |
| and related | |
| regulatory | |
| factors | |
| MnSOD, | Alzheimer's disease |
| transcription | |
| factors, | |
| and related | |
| regulatory | |
| factors | |
| MSH3 | Spinocerebellar ataxia |
| MSH3 | Myotonic dystrophy |
| MSH3 | Huntington's disease |
| MSLN | Mediastinal large B-cell |
| | lymphoma |
| MSLN | Metastatic non-small cell |
| | lung cancer |
| MSLN | Tumor |
| MSLN | Primary peritoneal cancer |
| MSLN | Pancreatic acinar cell |
| | carcinoma |
| CD3, CD86, | Bronchogenic carcinoma |
| PD-1 | |
| CD3, CD86, | Pancreatic cancer |
| PD-1 | |
| CD3, CD86, | Pharyngeal tumor |
| PD-1 | |
| CD3, CD86, | Small cell lung cancer |
| PD-1 | |
| CD3, CD86, | Gastrointestinal tumor |
| PD-1 | |
| CD3, CD86, | Gastric cancer |
| PD-1 | |
| CD3, CD86, | Advanced malignant solid |
| PD-1 | tumor |
| CD3, CD86, | Advanced cancer |
| PD-1 | |
| CD3, CD86, | Head and neck tumor |
| PD-1 | |
| CD3, CD86, | Fallopian tube cancer |
| PD-1 | |
| CD3, CD86, | Esophageal cancer |
| PD-1 | |
| CD3, CD86, | Kidney tumor |
| PD-1 | |
| CD3, CD86, | Soft tissue sarcoma |
| PD-1 | |
| CD3, CD86, | Breast cancer |
| PD-1 | |
| CD3, CD86, | Sarcoma |
| PD-1 | |
| CD3, CD86, | Female genitor tumor |
| PD-1 | |
| CD3, CD86, | Brain cancer |
| PD-1 | |
| CD3, CD86, | Ovarian cancer |
| PD-1 | |
| CD3, CD86, | Colorectal cancer |
| PD-1 | |
| CD3, CD86, | Laryngeal tumor |
| PD-1 | |
| CD3, CD86, | Melanoma |
| PD-1 | |
| CD3, CD86, | Osteosarcoma |
| PD-1 | |
| MSLN | Pancreatic adenocarcinoma |
| MSLN | Pancreatic cancer |
| MSLN | Adenocarcinoma |
| MSLN | Gastric cancer |
| MSLN | Advanced cancer |
| MSLN | Fallopian tube cancer |
| MSLN | Solid tumor |
| MSLN | Triple-negative breast |
| | cancer |
| MSLN | Breast cancer |
| MSLN | Refractory B-cell lymphoma |
| MSLN | Follicular lymphoma |
| MSLN | Ovarian adenocarcinoma |
| MSLN | Ovarian epithelial cancer |
| MSLN | Ovarian epithelial cancer |
| MSLN | Ovarian serous |
| | adenocarcinoma |
| MSLN | High-grade serous ovarian |
| | adenocarcinoma |
| MSLN | Ovarian cancer |
| MSLN | Ovarian cancer |
| MSLN | Colorectal cancer |
| MSLN | Mesothelioma |
| MSLN | Mesothelioma |
| MSLN | Malignant peritoneal |
| | mesothelioma |
| CD3, CD86, | Bone cancer |
| PD-1 | |
| CD3, CD86, | Cervical cancer |
| PD-1 | |
| CD3, CD86, | Liver cancer |
| PD-1 | |
| CD3, CD86, | Peritoneal cancer |
| PD-1 | |
| CD3, CD86, | Recurrent Head and neck |
| PD-1 | tumor |
| CD3, CD86, | Recurrent ovarian cancer |
| PD-1 | |
| CD3, CD86, | Lung cancer |
| PD-1 | |
| CD3, CD86, | Non-small cell lung cancer |
| PD-1 | |
| CD3, CD86, | Ear tumor |
| PD-1 | |
| CD3, CD86, | Malignant epithelial tumor |
| PD-1 | |
| CD3, CD86, | Nasal tumor |
| PD-1 | |
| CD3, EGFR | Pancreatic adenocarcinoma |
| CD3, EGFR | Pancreatic cancer |
| CD3, EGFR | Glioblastoma |
| CD3, | Glioblastoma |
| EGFRvIII, | |
| IL-13Rα2 | |
| CD3, | Tumor |
| EpCAM | |
| CD3, EphA2 | Tumor |
| CD3, FAP | Tumor metastasis |
| CD3, FAP | Adenoepithelial tumor |
| CD3, FAP | Malignant epithelial tumor |
| CD3, gp100 | Uveal melanoma |
| CD3, gp100 | Cutaneous melanoma |
| CD3, gp100 | Residual tumor |
| CD3, GPC3 | Solid tumor |
| CD3, | Tumor |
| GPRC5D | |
| MSLN | Lung cancer |
| MSLN | Non-small cell lung cancer |
| MSLN | Malignant pleural |
| | mesothelioma |
| MSLN | Cholangiocarcinoma |
| MSLN | Cholangiocarcinoma |
| MSLN | Diffuse large B-cell |
| | lymphoma |
| MSLN | Mesothelin-expressing |
| | tumor |
| MSLN | Mesothelin-expressing solid |
| | tumor |
| MSLN, | Pancreatic acinar cell |
| MUC16 | carcinoma |
| MSLN, | Ovarian epithelial cancer |
| MUC16 | |
| MSLN, | Advanced malignant solid |
| PD-1 | tumor |
| MSLN, | Breast cancer |
| PD-1 | |
| MSLN, | Mesothelioma |
| PD-1 | |
| MSLN, | Mesothelioma |
| PD-1 | |
| MSLN, | Mesothelin-expressing solid |
| PD-1 | tumor |
| MSLN, | Ovarian cancer |
| PDL1 | |
| MSP-1 | Malaria |
| MSTN | Muscle atrophy |
| MSTN | Duchenne muscular |
| | dystrophy |
| MSTN | Inclusion body myositis |
| MTM1 | Congenital myopathy |
| MTM1 | Rheumatoid arthritis |
| MTM1 | Muscle disorders |
| MT-ND1 | Leber Hereditary Optic |
| | Neuropathy, ND1 mutation |
| MT-ND4 | Leber Hereditary Optic |
| | Neuropathy, ND4 mutation |
| CD3, HER2 | Breast cancer |
| CD3, | Glioblastoma |
| IL-13Rα2, | |
| PDL1 | |
| CD3, | Glioma |
| IL-13Rα2, | |
| PDL1 | |
| CD3, MUC1 | Advanced pancreatic |
| | adenocarcinoma |
| CD3, MUC1 | Advanced gastric cancer |
| CD3, MUC1 | Advanced breast cancer |
| CD3, MUC1 | Kidney tumor |
| CD3, MUC1 | Colorectal cancer |
| CD3, MUC1 | Liver cancer |
| CD3, MUC1 | Lung cancer |
| CD3, PDL1 | Solid tumor |
| CD3, PDL1 | Recurrent cervical cancer |
| CD3, | Advanced malignant solid |
| PRAME | tumor |
| CD3, | Cutaneous melanoma |
| PRAME | |
| CD3, | Melanoma |
| PRAME | |
| CD3, | Refractory plasma cell |
| SLAMF7 | myeloma |
| CD3, | Relapsed Multiple myeloma |
| SLAMF7 | |
| CD30 | Peripheral T-cell lymphoma |
| CD30 | Diffuse large B-cell |
| | lymphoma |
| CD30 | Anaplastic large cell |
| | lymphoma |
| CD30 | Anaplastic large cell |
| | lymphoma |
| CD30 | Hodgkin lymphoma |
| CD30 | T-cell lymphoma |
| CD30 | CD30-positive lymphoma |
| CD30 | CD30-positive Hodgkin |
| | lymphoma |
| CD30 | CD30-positive diffuse large |
| | B-cell lymphoma |
| CD30 | CD19-positive lymphoma |
| CD30 | CD19 expressing malignancy |
| CD30 | B-cell lymphoma |
| CD30 | B-cell leukemia |
| CD33 | Graft-versus-host disease |
| mTOR | Tumor |
| mTOR | Hemophilia A |
| mTOR | Diabetic retinopathy |
| mTOR | Retinal disease |
| mTOR | Methylmalonic acidemia |
| MUC1 | Metastatic solid tumor |
| MUC1 | Metastatic breast cancer |
| MUC1 | Pancreatic acinar cell |
| | carcinoma |
| MUC1 | Pancreatic ductal carcinoma |
| MUC1 | Pancreatic cancer |
| MUC1 | Gastric cancer |
| MUC1 | Advanced gastric cancer |
| MUC1 | Advanced malignant solid |
| | tumor |
| MUC1 | Head and neck squamous |
| | cell carcinoma |
| MUC1 | Fallopian tube cancer |
| MUC1 | Esophageal cancer |
| MUC1 | Renal cell carcinoma |
| MUC1 | Breast cancer |
| MUC1 | Prostate cancer |
| MUC1 | Brain malignant glioma |
| MUC1 | Ovarian epithelial |
| | carcinoma |
| MUC1 | Ovarian cancer |
| MUC1 | Colorectal cancer |
| MUC1 | Glioma |
| MUC1 | Hepatocellular carcinoma |
| MUC1 | Intrahepatic |
| | cholangiocarcinoma |
| MUC1 | Lung cancer |
| MUC1 | Non-small cell lung cancer |
| MUC1 | Non-small cell lung cancer |
| MUC1 | Multiple myeloma |
| MUC1 | Nasopharyngeal tumor |
| CD33 | Acute myeloid leukemia |
| CD33 | Alzheimer's disease |
| CD33, CLL-1 | Hematologic malignancy |
| CD33, CLL-1 | Chronic myeloid leukemia |
| CD33, CLL-1 | Chronic myelomonocytic |
| | leukemia |
| CD33, CLL-1 | Acute myeloid leukemia |
| CD33, CLL-1 | Acute myeloid leukemia |
| CD33, CLL-1 | Myelodysplastic syndrome |
| CD33, FLT3 | Hematologic malignancy |
| CD33, FLT3 | Refractory acute myeloblastic |
| | leukemia |
| CD33, FLT3 | Myelodysplastic syndrome |
| CD33, FLT3 | Relapsed acute myeloblastic |
| | leukemia |
| CD33, | Acute myeloid leukemia |
| IL-15Rα, | |
| NKG2C | |
| CD33, | Acute myeloid leukemia in |
| NKG2A | adults |
| CD34 | Severe combined |
| | immunodeficiency |
| CD34 | Tumor |
| CD34 | Hematologic malignancy |
| CD34 | Adenosine deaminase |
| | deficiency |
| CD34 | Myeloid tumor |
| CD34 | Acute leukemia |
| CD34 | Myelodysplastic syndrome |
| CD34 | Multiple myeloma |
| CD34 | Wiskott-Aldrich syndrome |
| CD37 | Chronic lymphocytic |
| | leukemia |
| CD37 | T-cell lymphoma |
| CD37 | B-cell lymphoma |
| CD38 | Refractory acute myeloid |
| | leukemia |
| MUC1 | MUC1-expressing solid |
| | tumor |
| MUC16 | Breast cancer |
| MUC16 | Ovarian epithelial |
| | carcinoma |
| Mucin | Tumor |
| Mucin | Asthma |
| Mucin | Chronic obstructive |
| | pulmonary disease |
| MUSK | Myasthenia gravis |
| MUT | Methylmalonic acidemia |
| MUT, | Mesothelioma |
| mTOR | |
| MYB | Hematologic malignancy |
| MYB | Chronic myeloid leukemia |
| MYB | Leukemia |
| MYBPC3 | Hypertrophic |
| | cardiomyopathy |
| MYO6 | Hearing impairment |
| MYO7A | Usher syndrome |
| MYO7A | Usher syndrome type 1B |
| NaCT | Epilepsy |
| NAGA | Fabry disease |
| NAGLU | Lower urinary tract |
| | symptom |
| NAGLU | Mucopolysaccharidosis type |
| | III |
| Nav1.1 | Mucopolysaccharidosis type |
| | I |
| Nav1.1 | Rett syndrome |
| Nav1.1 | Dravet syndrome |
| Nav1.1 | CDKL5 Deficiency |
| Nav1.2 | Genetic disease |
| Nav1.2 | Precocious puberty |
| Nav1.2 | Epileptic encephalopathy |
| CD38 | Hepatocellular carcinoma |
| CD38 | Relapsed acute myeloid |
| | leukemia |
| CD38 | Multiple myeloma |
| CD38, CLL-1 | Cancer |
| CD38, DR5 | Multiple myeloma |
| CD38, | Tumor |
| GPRC5D | |
| CD39, CD73 | Rheumatoid arthritis |
| CD4 | Hematopoietic stem cell |
| | transplantation |
| CD4 | Graft-versus-host disease |
| CD4 | Precursor T-cell lymphocytic |
| | leukemia/lymphoma |
| CD4 | Chronic myelomonocytic |
| | leukemia |
| CD4 | T-cell lymphoma |
| CD4 | T-cell leukemia |
| CD4 | HIV infection |
| CD4, CD8 | Colorectal cancer |
| CD4, CD8 | HIV infection |
| CD40 | Autoimmune disease |
| CD40 | Tumor metastasis |
| CD40 | Adenoepithelial tumor |
| CD40 | Autoimmune neurological |
| | disorder |
| CD40 | Organ transplant rejection |
| CD40 | Hypersensitivity reactions |
| CD40L | Metastatic renal cell |
| | carcinoma |
| CD40L | Tumor |
| CD40L | Chronic lymphocytic |
| | leukemia |
| CD40L | Vaccination |
| CD40L | Glioma |
| CD40L | Melanoma |
| Nav1.2 | SCN2A-related |
| | encephalopathy |
| Nav1.6 | Developmental and epileptic |
| | encephalopathy |
| Nav1.6 | CDKL5 deficiency |
| Nav1.7 | Small fiber neuropathy |
| Nav1.7 | Pain |
| Nav1.7 | Neuralgia |
| Nav1.7 | Erythromelalgia |
| Nav1.7, | Pain |
| Nav1.8 | |
| Nav1.7, | Neuralgia |
| Voltage-gated | |
| sodium | |
| channels | |
| Nav1.8 | Pain |
| Nav1.8, | Neurological disorders |
| TRKA | |
| NCALD | Spinal muscular atrophy |
| NCF1 | Chronic granulomatous |
| | disease |
| NCL | Tumor |
| NCL | Tumor |
| NCL | Triple-negative breast |
| | cancer |
| NCL | Myelodysplastic syndrome |
| NCOR2 | Tumor |
| NCR2, | Advanced solid tumor |
| NKG2D | |
| NCR3LG1 | Tumor |
| NCR3LG1 | Solid tumor |
| NECTIN2 | Tumor |
| Nectin-4 | Tumor |
| NEGF2 | Tumor |
| NEK2 | Pancreatic cancer |
| NEK2 | Cholangiocarcinoma |
| NF-κB | Atopic dermatitis |
| NF-κB | Sepsis |
| CD40L | Recurrent non-small cell lung |
| | cancer |
| CD40L | Non-muscle invasive bladder |
| | tumor |
| CD40L | Bladder urothelial bladder |
| CD40L | Bladder cancer |
| CD40L | Stage IV malignant non-small |
| | cell lung tumor |
| CD40L, | Tumor |
| CD70, TLR4 | |
| CD40L, | Melanoma |
| CD70, TLR4 | |
| CD40L, | Stage IV malignant |
| CD70, TLR4 | melanoma |
| CD40L, | Unresectable melanoma |
| CD70, TLR4 | |
| CD40L, IFNβ | Tumor |
| CD40L, | Tumor |
| OX40L | |
| CD40L, | Triple-negative breast cancer |
| OX40L | |
| CD40L, | Sarcoma |
| OX40L | |
| CD40L, | Melanoma |
| OX40L | |
| CD40L, | Non-small cell lung cancer |
| OX40L | |
| CD43 | T-cell lymphoma |
| CD43 | T-cell leukemia |
| CD44 | Hematologic malignancy |
| CD44v6 | Solid tumor |
| CD44v6 | Acute myeloid leukemia |
| CD44v6 | Multiple myeloma |
| CD45 | Hematologic malignancy |
| CD45 | Bone marrow tumor |
| CD46 | Rectal cancer |
| CD46 | Head and neck tumor |
| CD46 | Renal cell carcinoma |
| CD46 | Ovarian cancer |
| CD46 | Colorectal cancer |
| CD46 | Non-small cell lung cancer |
| CD46 | Bladder cancer |
| CD46, PEDF | Geographic atrophy |
| NF-κB | Acute kidney injury |
| NF-κB | Obstetric delivery |
| | (premature birth) |
| NGF | Hearing loss |
| NGF | Retinitis pigmentosa |
| NGF | Retinal disease |
| NGF | Neuralgia |
| NGF | Glaucoma |
| NGF | Parkinson's disease |
| NGF | Stroke |
| NGF | Keratitis |
| NGF | Spinal cord injury |
| NGF | Macular degeneration |
| NGF | Huntington's disease |
| NGF | Osteoporosis |
| NGF | Osteoarthritis |
| NGF | Alzheimer's disease |
| NGLY1 | Genetic disease |
| NGLY1 | NGLY1 deficiency |
| Nicotine | Tobacco dependence |
| NIS | Pancreatic ductal |
| | adenocarcinoma |
| NIS | Fallopian tube cancer |
| NIS | Breast cancer |
| NIS | Prostate cancer |
| NIS | Ovarian cancer |
| NIS | Squamous cell carcinoma |
| NIS | Mesothelioma |
| NIS | Rhabdomyosarcoma |
| NIS | Peritoneal cancer |
| NIS | Multiple myeloma |
| NIS | Bladder cancer |
| NK1 | Hypertonia |
| CD47 | Hematologic malignancy |
| CD47 | Solid tumor |
| CD47 | Sickle cell disease |
| CD47 | Thalassemia |
| CD47, PHD2 | Tumor |
| CD47, PHD2 | Hematologic malignancy |
| CD47, PHD2 | Solid tumor |
| CD47, SIRPα | Tumor |
| CD47, SIRPα | Advanced malignant solid |
| | tumor |
| CD47, SIRPα | Solid tumor |
| CD47, SIRPα | Refractory non-Hodgkin |
| | lymphoma |
| CD47, SIRPα | Relapsed non-Hodgkin |
| | lymphoma |
| CD47, SIRPα | Atherosclerosis |
| CD49d | Multiple sclerosis |
| CD49d | Duchenne muscular |
| | dystrophy |
| CD5 | Mycosis fungoides |
| CD5 | Hematologic malignancy |
| CD5 | Peripheral T-cell lymphoma |
| CD5 | Precursor T-cell lymphocytic |
| | leukemia/lymphoma |
| CD5 | Precursor B-cell |
| | lymphoblastic |
| | leukemia/lymphoma |
| CD5 | Refractory T-cell acute |
| | lymphoblastic leukemia |
| CD5 | Refractory B-type acute |
| | lymphoblastic leukemia |
| CD5 | Lymphoma |
| CD5 | Acute lymphoblastic |
| | leukemia |
| CD5 | Relapsed acute lymphoblastic |
| | leukemia |
| CD5 | Relapsed T-cell lymphoma |
| CD5 | Non-Hodgkin lymphoma |
| CD5 | T-cell lymphoma |
| CD5 | T-cell acute lymphoblastic |
| | leukemia/lymphoma |
| NK2R, | Major depressive disorder |
| mGluRs | |
| NKG2D | Metastatic solid tumor |
| NKG2D | Rectal cancer |
| NKG2D | Hematologic malignancy |
| NKG2D | Novel Coronavirus Infection |
| NKG2D | Medulloblastoma |
| NKG2D | Medulloblastoma |
| NKG2D | Refractory acute myeloid |
| | leukemia |
| NKG2D | Colorectal cancer |
| NKG2D | Colon cancer liver |
| | metastasis |
| NKG2D | Colon cancer |
| NKG2D | Glioblastoma |
| NKG2D | Glioblastoma |
| NKG2D | Acute myeloid leukemia |
| NKG2D | Acute myeloid leukemia |
| NKG2D | Myelodysplastic syndrome |
| NKG2D | Hepatocellular carcinoma |
| NKG2D | Relapsed acute myeloid |
| | leukemia |
| NKG2D | Unresectable colorectal |
| | cancer |
| NKG2D | NKG2DL-positive solid |
| | tumor |
| NLGase | Gaucher disease |
| NLRP1, | Neurodegenerative disease |
| NLRP3 | |
| NLRP3 | Inflammatory bowel disease |
| NLRP3 | Inflammation |
| NMNAT1 | Leber congenital amaurosis |
| N-Myc | Small cell lung cancer |
| N-Myc | Neuroblastoma |
| N-Myc | Soft tissue sarcoma |
| NOD2 | Crohn's disease |
| CD5, IL15R | Hematologic malignancy |
| CD56 | Extranodal NK-T-cell |
| | lymphoma |
| CD56 | Recurrent nasopharyngeal |
| | carcinoma |
| CD59 | Age-related macular |
| | degeneration |
| CD59 | Geographic atrophy |
| CD7 | Juvenile idiopathic arthritis |
| CD7 | Mycosis fungoides |
| CD7 | Peripheral T-cell lymphoma |
| CD7 | Sezary syndrome |
| CD7 | Precursor T-cell lymphocytic |
| | leukemia/lymphoma |
| CD7 | Dermatomyositis |
| CD7 | Refractory T-cell |
| | lymphocytic/lymphoma |
| CD7 | Refractory T-cell acute |
| | lymphoblastic leukemia |
| CD7 | Immunoblastic |
| | lymphadenopathy |
| CD7 | Lymphoblastic lymphoma |
| CD7 | Ulcerative colitis |
| CD7 | Crohn's disease |
| CD7 | Extranodal NK-T-cell |
| | lymphoma |
| CD7 | Anaplastic large cell |
| | lymphoma |
| CD7 | Acute myeloid leukemia |
| CD7 | Acute lymphoblastic |
| | leukemia |
| CD7 | Hepatosplenic T-cell |
| | lymphoma |
| CD7 | Relapsed T-cell lymphoma |
| CD7 | Non-Hodgkin lymphoma |
| CD7 | Large granular lymphocytic |
| | leukemia |
| CD7 | Adult T-cell |
| | lymphoma/leukemia |
| NOS | Head and neck tumor |
| NOS | Prostate cancer |
| NOS | Liver cancer |
| NOS | Pulmonary hypertension due |
| | to lung disease |
| NOS | Pulmonary arterial |
| | hypertension |
| NOS2 | Vascular restenosis |
| NOS2 | Pulmonary arterial |
| | hypertension |
| NOX | Chronic granulomatous |
| | disease |
| NOX1 | Chronic granulomatous |
| | disease |
| NPC1 | Niemann-Pick disease type C |
| NPRA | Tumor |
| NPRA | Immune system disorder |
| NPY | Epilepsy |
| receptor | |
| NR2E3 | Retinitis pigmentosa |
| NR2E3 | Leber congenital amaurosis |
| NR2F6 | Tumor |
| NR2F6 | Solid tumor |
| NR2F6 | Myelodysplastic syndrome |
| NR2F6 | Leukemia |
| NRARP | Wet age-related macular |
| | degeneration |
| NRARP | Wet macular degeneration |
| NRARP | Choroidal |
| | neovascularization |
| NRARP | Macular degeneration |
| NRAS | Tumor |
| NRAS | Hematologic malignancy |
| NRAS | Solid tumor |
| CD7 | Adult T-cell |
| | leukemia/lymphoma |
| CD7 | Enteropathy-associated T-cell |
| | lymphoma |
| CD7 | Enteropathy-associated T-cell |
| | lymphoma |
| CD7 | T-cell lymphocytic leukemia |
| CD7 | T-cell lymphoma |
| CD7 | T-cell lymphoma |
| CD7 | Recurrent T-cell acute |
| | lymphoblastic leukemia |
| CD7 | T-cell acute lymphoblastic |
| | leukemia/lymphoma |
| CD7 | T-cell acute lymphoblastic |
| | leukemia/lymphoma |
| CD7 | NK-cell lymphoma |
| CD7 | CD7-positive tumor |
| CD7 | CD7-positive acute myeloid |
| | leukemia |
| CD70 | Metastatic clear cell renal cell |
| | carcinoma |
| CD70 | Tumor metastasis |
| CD70 | Pancreatic adenocarcinoma |
| CD70 | Hematologic malignancy |
| CD70 | Advanced renal cell |
| | carcinoma |
| CD70 | Advanced solid malignant |
| | tumor |
| CD70 | Advanced cancer |
| CD70 | Esophageal cancer |
| CD70 | Solid tumor |
| CD70 | Solid tumor |
| CD70 | Renal cell carcinoma |
| CD70 | Urologic tumor |
| CD70 | Ovarian cancer |
| CD70 | Lymphoma |
| CD70 | Acute myeloid leukemia |
| CD70 | Acute myeloid leukemia |
| CD70 | Cervical cancer |
| CD70 | Non-Hodgkin lymphoma |
| CD70 | Non-Hodgkin lymphoma |
| Nrf2 | Nonalcoholic steatohepatitis |
| Nrf2 | Arteriosclerosis |
| NRP-1 | Tumor |
| NRP-2 | Metastatic melanoma |
| NRP-2 | Tumor metastasis |
| NRP-2 | Tumor |
| NRP-2 | Kidney tumor |
| NRTN | Ocular disease |
| NRTN | Parkinson's disease |
| NRTN | Huntington's disease |
| NT-4 | Motor neurone disease |
| NT-4 | Hearing impairment |
| NT-4 | Diabetic retinopathy |
| NT-4 | Glaucoma |
| NT-4 | Uveitis |
| NT-4 | Parkinson's disease |
| NT-4 | Huntington's disease |
| NTCP | Liver disease |
| NTCP | Cholestasis |
| NTF3 | Charcot-Marie-Tooth |
| | Disease |
| Nucleic | Duchenne muscular |
| acids | dystrophy |
| NXNL1 | Cone-rod dystrophy |
| NXNL1 | Retinitis pigmentosa |
| NY-ESO-1 | Metastatic solid tumor |
| NY-ESO-1 | Tumor |
| NY-ESO-1 | Myxoid liposarcoma |
| NY-ESO-1 | Advanced solid malignant |
| | tumor |
| NY-ESO-1 | Esophageal squamous cell |
| | carcinoma |
| NY-ESO-1 | Esophageal cancer |
| NY-ESO-1 | Esophageal cancer |
| NY-ESO-1 | Solid tumor |
| CD70 | Malignant pleural |
| | mesothelioma |
| CD70 | Multiple myeloma |
| CD70 | T-cell lymphoma |
| CD70 | CD70-positive tumor |
| CD70 | CD70-positive female genitor |
| | tumor |
| CD70, IL-15, | Renal cell carcinoma |
| TGF-β | |
| CD70, IL15R | Advanced renal cell |
| | carcinoma |
| CD70, IL15R | Mesothelioma |
| CD70, IL15R | Osteosarcoma |
| CD72 | Acute lymphoblastic |
| | leukemia |
| CD74 | Alzheimer's disease |
| CD79B | Refractory B-cell acute |
| | lymphoblastic leukemia |
| CD79B | Acute lymphoblastic |
| | leukemia |
| CD79B | B-cell lymphoma |
| CD79B | B-cell lymphoma |
| CD8 | Advanced Head and neck |
| | tumor |
| CD8 | Head and neck tumor |
| CD80, CD86, | Non-small cell lung cancer |
| PDL1 | |
| CD80, | Tumor |
| GM-CSF | |
| CD80, IFNα, | Solid tumor |
| MIP-1α | |
| CD83 | Tumor |
| CD84 | Adult acute myeloid |
| | leukemia |
| CD86 | Glioma |
| CD86 | Melanoma |
| CD86, HPV | Solid tumor |
| E6, HPV E7, | |
| IL-12, IL-2 | |
| NY-ESO-1 | Solid tumor |
| NY-ESO-1 | Neuroblastoma |
| NY-ESO-1 | Triple-negative breast |
| | cancer |
| NY-ESO-1 | Soft tissue sarcoma |
| NY-ESO-1 | Breast cancer |
| NY-ESO-1 | Breast cancer |
| NY-ESO-1 | Sarcoma |
| NY-ESO-1 | Meningioma |
| NY-ESO-1 | Refractory ovarian cancer |
| NY-ESO-1 | Epithelial ovarian |
| | carcinoma |
| NY-ESO-1 | Ovarian cancer |
| NY-ESO-1 | Synovial sarcoma |
| NY-ESO-1 | Synovial sarcoma |
| NY-ESO-1 | Melanoma |
| NY-ESO-1 | Hepatocellular carcinoma |
| NY-ESO-1 | Liver cancer |
| NY-ESO-1 | Recurrent primary |
| | peritoneal cancer |
| NY-ESO-1 | Lung cancer |
| NY-ESO-1 | Non-small cell lung cancer |
| NY-ESO-1 | Multiple myeloma |
| NY-ESO-1 | Platinum-resistant fallopian |
| | tube cancer |
| NY-ESO-1 | Platinum-resistant ovarian |
| | cancer |
| NY-ESO-1 | Platinum-sensitive primary |
| | peritoneal carcinoma |
| NY-ESO-1 | Platinum-sensitive fallopian |
| | tube cancer |
| NY-ESO-1 | Platinum-resistant primary |
| | peritoneal cancer |
| CD86, HPV | HPV16-positive solid tumor |
| E6, HPV E7, | |
| IL-12, IL-2 | |
| CD8A, | Solid tumor |
| NY-ESO-1 | |
| CD94 | Plasmacytic leukemia |
| CD99 | Hematologic malignancy |
| CD99 | Myeloid leukemia |
| CD99 | Sarcoma |
| CDA | Gastrointestinal tumor |
| CDA | Glioblastoma |
| CDA | High-grade glioma |
| CDA | Recurrent glioblastoma |
| CDCP1 | Pancreatic ductal |
| | adenocarcinoma |
| CDH17 | Metastatic colorectal cancer |
| CDH17 | Pancreatic cancer |
| CDH17 | Neuroendocrine tumor |
| CDH17 | Colorectal cancer |
| CDH5 | Diabetic retinopathy |
| CDKL5 | Neurological disorder |
| CDKL5 | X-linked genetic disease |
| CDKL5 | CDKL5 Deficiency |
| CDKN1A | Non-muscle invasive bladder |
| | tumor |
| CDKs | Tumor |
| CDKs | Sensorineural hearing loss |
| CEA | Tumor metastasis |
| CEA | Rectal cancer |
| CEA | Pancreatic acinar carcinoma |
| CEA | Pancreatic cancer |
| NY-ESO-1 | Urothelial bladder cancer |
| NY-ESO-1 | Bladder cancer |
| NY-ESO-1 | FGF19-positive solid tumor |
| NY-ESO-1, | Myxoid liposarcoma |
| PD-1 | |
| NY-ESO-1, | Synovial sarcoma |
| PD-1 | |
| NY-ESO-1, | Melanoma |
| PD-1 | |
| NY-ESO-1, | Multiple myeloma |
| PD-1 | |
| NY-ESO-1, | Acute myeloid leukemia |
| PRAME, | |
| WT1 | |
| NY-ESO-1, | Acute lymphoblastic |
| PRAME, | leukemia |
| WT1 | |
| NY-ESO-1, | Solid tumor |
| TCR | |
| NY-ESO-1, | Myxoid liposarcoma |
| TGF-β | |
| NY-ESO-1, | Synovial sarcoma |
| TGF-β | |
| OPA1 | Optic atrophy |
| OPA1 | Autosomal dominant optic |
| | atrophy |
| OPCML | Ovarian cancer |
| OTCase | Urea cycle disorder |
| OTCase | Ornithine transcarbamylase |
| | deficiency |
| OTOF | Hearing loss |
| OTOF | Deafness |
| OX40 | Metastatic colorectal cancer |
| OX40 | Gastrointestinal stromal |
| | tumor |
| OX40 | Gastric cancer |
| OX40 | Kidney tumor |
| OX40 | Breast cancer |
| OX40 | Sarcoma |
| OX40 | Squamous cell carcinoma |
| CEA | Gastric cancer |
| CEA | Advanced solid malignant |
| | tumor |
| CEA | Esophageal cancer |
| CEA | Oligodendroglioma |
| CEA | Breast cancer |
| CEA | Ovarian cancer |
| CEA | Colorectal cancer |
| CEA | Colorectal cancer |
| CEA | Colon cancer |
| CEA | Colon cancer |
| CEA | Liver metastasis |
| CEA | Liver cancer |
| CEA | Peritoneal cancer |
| CEA | Recurrent cancer |
| CEA | Lung cancer |
| CEA | Cholangiocarcinoma |
| CEA | Biliary tract tumor |
| CEA, | Colorectal cancer |
| IL-15Rα, | |
| IL-21R | |
| CEACAM5 | Pancreatic acinar carcinoma |
| CEACAM5 | Pancreatic adenocarcinoma |
| CEACAM5 | Pancreatic cancer |
| CEACAM5 | Adenocarcinoma |
| CEACAM5 | Gastric cancer |
| CEACAM5 | Gastric cancer |
| CEACAM5 | Breast cancer |
| CEACAM5 | Breast cancer |
| CEACAM5 | Ovarian cancer |
| CEACAM5 | Squamous non-small cell |
| | lung cancer |
| CEACAM5 | Colorectal cancer |
| CEACAM5 | Colorectal cancer |
| CEACAM5 | Liver metastasis |
| CEACAM5 | Liver cancer |
| CEACAM5 | Lung cancer |
| CEACAM5 | Malignant ascites |
| CEACAM5, | Tumor |
| CEACAM6 | |
| OX40 | Colorectal cancer liver |
| | metastasis |
| OX40 | Melanoma |
| OX40 | Liver metastasis |
| OX40 | Abdominal tumor |
| OX40L | Lymphoma |
| p24 antigen | HIV infection |
| p53 | Delayed recovery of graft |
| | function |
| p53 | Transplant complication |
| p53 | Pancreatic cancer |
| p53 | Small lymphocytic |
| | lymphoma |
| p53 | Advanced solid malignant |
| | tumor |
| p53 | Advanced bile duct cancer |
| p53 | Head and neck tumor |
| p53 | Fallopian tube cancer |
| p53 | Leukoplakia |
| p53 | Chronic lymphocytic |
| | leukemia |
| p53 | Ovarian cancer |
| p53 | Glioblastoma |
| p53 | Acute myeloid leukemia |
| p53 | Acute kidney injury |
| p53 | Myelodysplastic syndrome |
| p53 | Liver cancer |
| p53 | Peritoneal cancer |
| p53 | Non-small cell lung cancer |
| p53 | Malignant ascites |
| p53 | Nasopharyngeal carcinoma |
| p53 | Bladder cancer |
| p53 | Vitiligo |
| p53 R175H | Metastatic solid tumor |
| p53 R175H | Pancreatic adenocarcinoma |
| p53 R175H | Head and Neck Squamous |
| | Cell Carcinoma |
| p53 R175H | Breast cancer |
| p53 R175H | Colorectal cancer |
| p53 R175H | Non-small cell lung cancer |
| p75NTR | Alzheimer's disease |
| CEACAM7 | Pancreatic cancer |
| CEBPA | Hepatitis B |
| CEBPA | Hepatocellular carcinoma |
| CEBPA | Liver cancer |
| CEBPA | Hepatitis C |
| CEBPB | Breast cancer |
| CEBPB | Melanoma |
| CEBPB | Hepatocellular carcinoma |
| CEBPB | Bladder cancer |
| Cell adhesion | Mantle cell lymphoma |
| molecules, | |
| NF-κB, | |
| eIF5A | |
| Cell adhesion | Diffuse large B-cell |
| molecules, | lymphoma |
| NF-κB, | |
| eIF5A | |
| Cell adhesion | Plasma cell leukemia |
| molecules, | |
| NF-κB, | |
| eIF5A | |
| Cell adhesion | Relapsed mantle cell |
| molecules, | lymphoma |
| NF-κB, | |
| eIF5A | |
| Cell adhesion | Relapsed diffuse large B-cell |
| molecules, | lymphoma |
| NF-κB, | |
| eIF5A | |
| Cell adhesion | Relapsed Multiple myeloma |
| molecules, | |
| NF-κB, | |
| eIF5A | |
| Cell adhesion | Relapsed B-cell lymphoma |
| molecules, | |
| NF-κB, | |
| eIF5A | |
| PA protein, | Influenza virus infection |
| influenza | |
| virus M1 | |
| PABPN1 | Oculopharyngeal muscular |
| | dystrophy |
| PABPN1 | Swallowing disorders |
| PAH | Phenylketonuria |
| PAI-1, tPA, | Thrombotic |
| vWF | microangiopathy |
| PAI-1, tPA, | Novel Coronavirus Infection |
| vWF | |
| PAI-1, tPA, | Veno-occlusive disease |
| vWF | |
| PAI-1, tPA, | High-risk neuroblastoma |
| vWF | |
| PAI-1, tPA, | Hepatic veno-occlusive |
| vWF | disease |
| PAK4 | Non-muscle invasive |
| | bladder tumor |
| PAK4 | Bladder cancer |
| PARP | Tumor |
| PARP | Solid tumor |
| PARP1 | Tumor |
| PAX4 | Type 1 diabetes |
| PBGD | Acute intermittent porphyria |
| Cell adhesion | Multiple myeloma |
| molecules, | |
| NF-κB, | |
| eIF5A | |
| Cep290 | Retinal Degenerative Disease |
| Cep290 | Leber congenital amaurosis |
| | type 10 |
| Cep290, | Hereditary retinal dystrophy |
| nuclease | |
| Cas9 | |
| Cep290, | Eye deformities |
| nuclease | |
| Cas9 | |
| Cep290, | Retinal degeneration |
| nuclease | |
| Cas9 | |
| Cep290, | Visual impairment |
| nuclease | |
| Cas9 | |
| CFB | Age-related macular |
| | degeneration |
| CFB | Immune system disorders |
| CFB | Immunoglobulin A |
| | nephropathy |
| CFB | Geographic atrophy |
| CFH | Macular degeneration |
| CFI | Sagging skin |
| CFI | Age-related macular |
| | degeneration |
| CFI | Macular degeneration |
| CFI | Dry age-related macular |
| | degeneration |
| CFI | Geographic atrophy |
| CFI | Glycogen storage disease |
| | type II |
| CFLAR | Tumor |
| CFTR | Retinal disease |
| CFTR | Cystic fibrosis |
| cGMP-PDE | Retinitis pigmentosa |
| PCSK9 | Lipid metabolism disorder |
| PCSK9 | Heterozygous familial |
| | hypercholesterolemia |
| PCSK9 | Primary |
| | hypercholesterolemia |
| PCSK9 | Hereditary Leiomyomatosis |
| | and Renal Cell Carcinoma |
| PCSK9 | Peripheral arterial disease |
| PCSK9 | Stroke |
| PCSK9 | Familial combined |
| | hyperlipidemia |
| PCSK9 | Coronary artery disease |
| PCSK9 | Hyperlipidemia |
| PCSK9 | hypertension |
| PCSK9 | Hypercholesterolemia |
| PCSK9 | Liver disease |
| PCSK9 | Atherosclerosis |
| PCSK9 | Homozygous familial |
| | hypercholesterolemia |
| PCSK9 | Type IIa |
| | hyperlipoproteinemia |
| PD-1 | Metastatic melanoma |
| PD-1 | Metastatic non-small cell |
| | lung cancer |
| PD-1 | Advanced non-small cell |
| | lung cancer |
| PD-1 | Advanced solid malignant |
| | tumor |
| PD-1 | Esophageal cancer |
| PD-1 | Triple-negative breast |
| | cancer |
| PD-1 | Skin tumor |
| Chk1 | Brain metastases |
| Chk1 | Glioblastoma |
| chloride | Cystic fibrosis |
| channel | |
| CHOP | Diabetic nephropathy |
| CHST15 | Ulcerative colitis |
| CHST15 | Crohn's disease |
| chymase | Heart failure |
| CIDEB | Nonalcoholic steatohepatitis |
| CISH | Gastrointestinal tumor |
| CISH, IL-15 | Hematologic malignancy |
| CISH, IL-15 | Solid tumor |
| CISH, | Solid tumor |
| TGFBR2 | |
| c-Jun | Basal cell carcinoma |
| c-Jun | Melanoma |
| c-Kit | Myocardial ischemia |
| c-Kit | Systemic mastocytosis |
| c-Kit | allergy |
| CLCN7 | Autosomal dominant |
| | osteopetrosis type II |
| CLDN18.2 | Autoimmune disease |
| CLDN18.2 | Pancreatic cancer |
| CLDN18.2 | Gastric adenocarcinoma |
| CLDN18.2 | Gastroesophageal junction |
| | adenocarcinoma |
| CLDN18.2 | Gastrointestinal tumor |
| CLDN18.2 | Gastric cancer |
| CLDN18.2 | Advanced solid malignant |
| | tumor |
| CLDN18.2 | Solid tumor |
| CLDN18.2 | Ovarian epithelial carcinoma |
| CLDN18.2 | Ovarian cancer |
| CLDN18.2 | Advanced gastric |
| | adenocarcinoma |
| CLDN18.2 | CLDN18.2-positive |
| | pancreatic cancer |
| PD-1 | Squamous cell carcinoma of |
| | the skin |
| PD-1 | Skin melanoma |
| PD-1 | Brain metastases |
| PD-1 | Refractory cancer |
| PD-1 | Urinary system tumor |
| PD-1 | Merkel cell carcinoma |
| PD-1 | Ovarian cancer |
| PD-1 | Colorectal cancer |
| PD-1 | Glioma |
| PD-1 | Mesothelioma |
| PD-1 | Melanoma |
| PD-1 | Hepatocellular carcinoma |
| PD-1 | Non-small cell lung cancer |
| | stage III |
| PD-1 | Non-small cell lung cancer |
| PD-1 | Glioblastoma multiforme |
| PD-1 | Unresectable melanoma |
| PD-1 | Mesothelin-positive tumor |
| PD-1 | Mesothelin-positive solid |
| | tumor |
| PD-1 | IV Malignant Non-Small |
| | Cell Lung Tumor |
| PD-1 | Stage III colon cancer |
| PD-1 | Stage IIA non-small cell |
| | lung cancer |
| PD-1 | Carney complex |
| PD-1, | Prostate cancer |
| PSCA, | |
| PSMA | |
| PDCD5 | Leukemia |
| PDE4B, | Chronic obstructive |
| PDE4D, | pulmonary disease |
| PDE7A | |
| PDGFB | diabetic foot |
| PDGFB, | Macular degeneration |
| VEGFR | |
| PDGFR | Tumor |
| PDGFR | Ocular disease |
| PDGFR | Fibrosis |
| CLDN18.2 | CLDN18.2-positive |
| | gastroesophageal junction |
| | adenocarcinoma |
| CLDN18.2 | CLDN18.2-positive |
| | gastrointestinal tumor |
| CLDN18.2, | Pancreatic ductal |
| CXCR4 | adenocarcinoma |
| CLDN18.2, | Pancreatic cancer |
| GluN2B, | |
| NAMPT, | |
| NKG2D, | |
| NMDA | |
| receptor | |
| CLDN18.2, | Gastric cancer |
| GluN2B, | |
| NAMPT, | |
| NKG2D, | |
| NMDA | |
| receptor | |
| CLDN18.2, | NKG2DL-positive solid |
| GluN2B, | tumor |
| NAMPT, | |
| NKG2D, | |
| NMDA | |
| receptor | |
| CLDN18.2, | CLDN18.2-positive solid |
| GluN2B, | tumor |
| NAMPT, | |
| NKG2D, | |
| NMDA | |
| receptor | |
| CLDN18.2, | Pancreatic cancer |
| IL-15 | |
| CLDN18.2, | Melanoma |
| IL-15 | |
| CLDN18.2, | Gastric cancer |
| PD-1 | |
| CLDN18.2, | CLDN18.2-positive solid |
| PDL1 | tumor |
| CLDN6 | Solid tumor |
| CLDN6 | Ovarian cancer |
| CLDN6 | Testicular tumor |
| CLEC4K | Langerhans cell histiocytosis |
| CLL-1 | Chronic lymphocytic |
| | leukemia |
| CLL-1 | Acute myeloid leukemia |
| PDGFR | Hippel-Lindau syndrome |
| PDGFR | Retinoblastoma |
| PDGFR | Wet age-related macular |
| | degeneration |
| PDGFR | Wet macular degeneration |
| PDGFR | Macular degeneration |
| PDGFR, | Wet age-related macular |
| VEGFR1 | Degeneration |
| PDGFRα | Glioma |
| PDGFRβ | Cirrhosis |
| PDHK1 | Allergic conjunctivitis |
| PDL1 | Autoimmune hepatitis |
| PDL1 | Metastatic head and neck |
| | squamous cell carcinoma |
| PDL1 | Tumor metastasis |
| PDL1 | Tumor |
| PDL1 | Hepatitis B |
| PDL1 | Graft-versus-host disease |
| PDL1 | Pancreatic ductal |
| | adenocarcinoma |
| PDL1 | Pancreatic cancer |
| CLL-1 | Relapsed acute myeloid |
| | leukemia |
| CLN3 | Neuronal ceroid |
| | lipofuscinosis |
| CLN5 | Neuronal ceroid |
| | lipofuscinosis 5 |
| CLN6 | Neuronal ceroid |
| | lipofuscinosis |
| CLN8 | Neuronal ceroid |
| | lipofuscinosis |
| CLRN1 | Usher syndrome |
| CLU | Metastatic castration-resistant |
| | prostate cancer |
| CLU | Metastatic non-small cell |
| | lung cancer |
| CLU | Pain |
| CLU | Castration-resistant prostate |
| | cancer |
| CLU | Non-small cell lung cancer |
| c-Met | Foot ulcer |
| c-Met | Perioperative ischemia |
| c-Met | Solid tumor |
| c-Met | Ulcer |
| c-Met | Cirrhosis |
| c-Met | Atherosclerosis obliterans |
| c-Met | Hyperlipoproteinemia type I |
| CMV IE2 | Cytomegalovirus retinitis |
| CMV IE2 | Cytomegalovirus infection |
| CMV IE2 | HIV infection |
| CMV pp65, | Glioblastoma |
| LAMP-1 | |
| CMV protein | Adenoviridae Infections |
| CMV protein | Organ transplant rejection |
| CMV protein | Cytomegalovirus infection |
| PDL1 | Gastric cancer |
| PDL1 | Triple-negative breast |
| | cancer |
| PDL1 | Ovarian epithelial |
| | carcinoma |
| PDL1 | Locally advanced malignant |
| | solid tumor |
| PDL1 | Glioblastoma |
| PDL1 | Hodgkin lymphoma |
| PDL1 | Melanoma |
| PDL1 | Peritoneal cancer |
| PDL1 | Recurrent squamous cell |
| | carcinoma of the head and |
| | neck |
| PDL1, | Tumor |
| TLR9 | |
| PDL1, | Tumor |
| VEGFR1 | |
| PDL1, | Malignant ascites |
| VEGFR1 | |
| PECAM1 | Solid tumor |
| PECAM1 | Lung damage |
| PEDF | Retinitis pigmentosa |
| PfCSP | Plasmodium falciparum |
| PGF, | Diabetic macular edema |
| VEGF-A, | |
| VEGF-B, | |
| VEGF-C | |
| PGF, | Wet age-related macular |
| VEGF-A, | degeneration |
| VEGF-B, | |
| VEGF-C | |
| P-gp | Breast cancer |
| P-gp | Non-Hodgkin lymphoma |
| P-gp | Bladder cancer |
| PGRN | Frontotemporal dementia |
| PhD2 | Anemia |
| PhD2 | Acute kidney injury |
| Phosphotransferases | Restenosis |
| CMV protein | Polyomavirus infection |
| CMV protein | Epstein-Barr virus infection |
| CMV protein, | Infect |
| EBV protein | |
| c-Myc | Reperfusion injury |
| c-Myc | Primitive neuroectodermal |
| | tumor |
| c-Myc | Islet cell adenoma |
| c-Myc | Solid tumor |
| c-Myc | Lymphoma |
| c-Myc | Colorectal cancer |
| c-Myc | Huntington's disease |
| c-Myc | Coronary artery restenosis |
| c-Myc | Hepatocellular carcinoma |
| c-Myc | Non-small cell lung cancer |
| c-Myc | Non-Hodgkin lymphoma |
| c-Myc | Multiple myeloma |
| c-Myc | C-Myc positive solid tumor |
| CNGA3 | Color vision impairment |
| CNGA3 | Achromatopsia type 1 |
| CNGB1 | Retinitis pigmentosa |
| CNGB3 | Color vision impairment |
| CNGB3 | Color vision impairment |
| CNGB3 | Achromatopsia type 1 |
| CNTF | Retinitis pigmentosa |
| CNTFR | Autism Spectrum Disorders |
| CNTFR | Autism |
| CNTFR | Peripheral nerve damage |
| CNTFR | Retinitis pigmentosa |
| CNTFR | Retinal telangiectasia |
| Phosphotransferases, | Tumor |
| p38α | |
| Phosphotransferases, | Restenosis |
| p38α | |
| PI3K | Solid tumor |
| family, | |
| mTORC1, | |
| mTORC2 | |
| PI3Kα | Tumor |
| PI3Kα | Tumor |
| H1047R | |
| PI3Kα | Advanced breast cancer |
| H1047R | |
| PI3Kα | Advanced solid malignant |
| H1047R | tumor |
| PI3Kα | Advanced cancer |
| H1047R | |
| PI3Kα | Breast cancer |
| H1047R | |
| PIGF, | Wet macular degeneration |
| VEGF-A, | |
| VEGF-B | |
| PIKFYVE | Amyotrophic lateral |
| | sclerosis |
| PIKFYVE | Frontotemporal dementia |
| PIWIL1 | Pancreatic cancer |
| PIWIL1 | Gastrointestinal tumor |
| PIWIL1 | Colorectal cancer |
| PKA | Tumor |
| PKA | Solid tumor |
| PKA | Breast cancer |
| PKA | Ovarian cancer |
| PKA | Colon cancer |
| PKA | Lung cancer |
| PKCα | Lung cancer |
| PKCα | Non-small cell lung cancer |
| PKLR | Erythrocyte pyruvate kinase |
| | deficiency |
| PKN3 | Pancreatic cancer |
| PKN3 | Advanced solid malignant |
| | tumor |
| PKN3 | Head and neck tumor |
| PLA2 | Tumor |
| CNTFR | Optic nerve disease |
| CNTFR | Color vision impairment |
| CNTFR | Glaucoma |
| CNTFR | Parkinson's disease |
| CNTFR | Acute lung injury |
| CNTFR | Amyotrophic lateral sclerosis |
| CNTFR | Huntington's disease |
| CNTFR | Multiple sclerosis |
| CNTFR | Geographic atrophy |
| CNTFR | Usher syndrome |
| Coagulation | Coagulation disorder |
| factor | |
| COL1A1 | Skin aging |
| COL3A1 | Ehlers-Danlos syndrome |
| COL3A1, | Skin disorders |
| elastin | |
| COL5A1 | Ehlers-Danlos syndrome |
| COL6A3 | Solid tumor |
| COL7A1 | Dystrophic epidermolysis |
| | bullosa |
| COL7A1 | Graft-versus-host disease |
| COL7A1 | Burn |
| COL7A1 | Epidermolysis bullosa |
| exon 80 | |
| Collagen | Dystrophic epidermolysis |
| | bullosa |
| Collagen | Penile edema |
| Collagen | Lower limb ulcers |
| Collagen | Knee Injury |
| Collagen | Tennis elbow |
| Collagen | Cartilage disease |
| Collagen | Tendon injuries |
| Collagen | Tendinopathy |
| Collagen | Osteoarthritis |
| Collagen | Epidermolysis bullosa |
| COP1 | Hepatocellular carcinoma |
| PLA2G6 | Neuroaxonal dystrophy |
| PLA2R | Membranous |
| | Glomerulonephritis |
| plakophilin- | Arrhythmic right ventricular |
| 2 | dysplasia |
| plakophilin- | Myocardial disease |
| 2 | |
| PLK1 | Advanced hepatocellular |
| | carcinoma |
| PLK1 | Advanced solid malignant |
| | tumor |
| PLK1 | Adrenal cortical carcinoma |
| PLK1 | Neuroendocrine tumor |
| PLK1 | Lymphoma |
| PLK1 | Hepatocellular carcinoma |
| PLK4 | Solid tumor |
| PLK4 | Breast cancer |
| PLP1 | Pelizaeus-Merzbacher |
| | disease |
| PMP22 | Charcot-Marie-Tooth |
| | Disease Type Ia |
| PMVK | Pancreatic cancer |
| PMVK | Non-small cell lung cancer |
| PNP | Pharyngeal tumor |
| PNP | Advanced Head and neck |
| | tumor |
| PNP | Head and neck tumor |
| PNP | Prostate cancer |
| PNP | Squamous cell carcinoma |
| PNPLA3 | Fatty liver |
| PNPLA3 | Cardiovascular disease |
| PNPLA3 | Fibrosis |
| PNPLA3 | hepatitis |
| PNPLA3 | Nonalcoholic steatohepatitis |
| PNPLA3 | Metabolic disease |
| PNPLA3 | Metabolic fatty liver disease |
| POSTN | Diabetic retinopathy |
| Potassium | Urge incontinence |
| channel | |
| Potassium | Overactive bladder |
| channel | |
| COPS5, | Hepatitis B |
| WEE1 | |
| COX-2 | Prostate cancer |
| COX-2 | Basal cell carcinoma |
| COX-2, | Colon cancer |
| PGE2 | |
| COX-2, | Liver cancer |
| PGE2 | |
| COX-2, | Carcinoma in situ |
| TGF-β1 | |
| COX-2, | Primary sclerosing |
| TGF-β1 | cholangitis |
| COX-2, | Solid tumor |
| TGF-β1 | |
| COX-2, | Breast cancer |
| TGF-β1 | |
| COX-2, | Prostate cancer |
| TGF-β1 | |
| COX-2, | Hypertrophic scars of the |
| TGF-β1 | skin |
| COX-2, | Squamous cell carcinoma of |
| TGF-β1 | the skin |
| COX-2, | Local obesity |
| TGF-β1 | |
| COX-2, | Basal cell carcinoma |
| TGF-β1 | |
| COX-2, | Liver metastasis |
| TGF-β1 | |
| COX-2, | Cirrhosis |
| TGF-β1 | |
| COX-2, | Hepatocellular carcinoma |
| TGF-β1 | |
| COX-2, | Obesity |
| TGF-β1 | |
| COX-2, | Cholangiocarcinoma |
| TGF-β1 | |
| COX-2, | Keloid |
| TGF-β1 | |
| CRAF | Tumor |
| CRAF | Pancreatic cancer |
| CRAF | Small cell lung cancer |
| PPAR | Acne vulgaris |
| PPT1 | Neuronal ceroid |
| | lipofuscinosis |
| PR1 | Acute myeloid leukemia in |
| | adults |
| PRAME | Endometrial cancer |
| PRAME | Tumor |
| PRAME | Head and neck tumor |
| PRAME | Solid tumor |
| PRAME | Uveal melanoma |
| PRAME | Skin melanoma |
| PRAME | Ovarian cancer |
| PRAME | Acute myeloid leukemia |
| PRAME | Synovial sarcoma |
| PRAME | Melanoma |
| PRAME | Myelodysplastic syndrome |
| PRAME | Myelodysplastic syndrome |
| PRAME | Non-small cell lung cancer |
| PRAME, | Prostate cancer |
| WT1 | |
| PRAME, | Acute myeloid leukemia |
| WT1, cyclin | |
| A1 | |
| PRAME, | Myelodysplastic syndrome |
| WT1, cyclin | |
| A1 | |
| PRDM2 | Prostate cancer |
| PRDM2 | Liver cancer |
| PRDM2 | Non-small cell lung cancer |
| PRG4 | Osteoarthritis |
| CRAF | Diabetic macular edema |
| CRAF | Solid tumor |
| CRAF | Renal cell carcinoma |
| CRAF | Breast cancer |
| CRAF | Prostate cancer |
| CRAF | Ovarian cancer |
| CRAF | Colorectal cancer |
| CRAF | Macular degeneration |
| CRAF | Non-small cell lung cancer |
| CRP | Inflammation |
| CRP | Atrial fibrillation |
| CRP | Kidney disease |
| CRP | Rheumatoid arthritis |
| CRP | Crohn's disease |
| CRX | Cone-rod dystrophy |
| CRX | Retinitis pigmentosa |
| CRX | Retinal disease |
| CRX | Leber congenital amaurosis |
| CSF-2R | Metastatic solid tumor |
| CSF-2R | Metastatic breast cancer |
| CSF-2R | Metastatic biliary tract cancer |
| CSF-2R | Tumor |
| CSF-2R | Central nervous system tumor |
| CSF-2R | Mucinous adenocarcinoma |
| CSF-2R | Ewing sarcoma |
| CSF-2R | Signet ring cell carcinoma |
| CSF-2R | Secondary malignant tumor |
| | of the pancreas |
| CSF-2R | Mycosis fungoides |
| CSF-2R | Eccrine sweat gland pore |
| | carcinoma |
| CSF-2R | Gastric cancer |
| CSF-2R | Microcystic adnexal |
| | carcinoma |
| CSF-2R | Advanced colorectal |
| | adenocarcinoma |
| PRNP | Prion disease |
| Protein | Tumor |
| kinases | |
| PRPF31 | Retinal degenerative disease |
| | associated with biallelic |
| | RPE65 mutations |
| PRPF31 | Retinitis pigmentosa type 11 |
| PSCA | Esophageal adenocarcinoma |
| PSCA | Bladder cancer |
| P-sel | Sickle cell disease |
| PSMA | Autoimmune disease |
| PSMA | Metastatic |
| | castration-resistant prostate |
| | cancer |
| PSMA | Tumor |
| PSMA | Mucoepidermoid carcinoma |
| PSMA | Adenoid cystic carcinoma |
| PSMA | Acinar cell carcinoma |
| PSMA | Salivary gland cancer |
| PSMA | Salivary gland tumor |
| PSMA | Renal cell carcinoma |
| PSMA | Breast cancer |
| PSMA | Castration-resistant prostate |
| | cancer |
| PSMA | Castration-resistant prostate |
| | cancer |
| PSMA | Prostate cancer |
| PSMA | Prostate cancer |
| PSMA | Colorectal cancer |
| PSMA | Non-small cell lung cancer |
| PSMA | Multiple myeloma |
| PSMA | Ductal carcinoma |
| PSMA | PSMA-positive tumor |
| PSMA, | Metastatic |
| TCRDV2 | castration-resistant prostate |
| | cancer |
| PSMA, | Castration-resistant prostate |
| TGF-β | cancer |
| PSMA, | Prostate cancer |
| TGF-β | |
| PSMA, | Prostate cancer |
| Trop-2 | |
| PTEN | Severe Acute Respiratory |
| | Syndrome |
| PTEN | Spinal cord injury |
| CSF-2R | Vulvar squamous cell |
| | carcinoma |
| CSF-2R | Head and neck tumor |
| CSF-2R | Retinoblastoma |
| CSF-2R | Esophageal cancer |
| CSF-2R | Solid tumor |
| CSF-2R | Wilms tumor |
| CSF-2R | Neuroblastoma |
| CSF-2R | Sezary syndrome |
| CSF-2R | Extramammary Paget's |
| | disease |
| CSF-2R | Ductal carcinoma |
| CSF-2R | Papillary adenocarcinoma |
| CSF-2R | Prostate cancer |
| CSF-2R | Sebaceous adenoma |
| CSF-2R | Skin tumor |
| CSF-2R | Squamous cell carcinoma of |
| | the skin |
| CSF-2R | Skin melanoma |
| CSF-2R | Skin adnexal cancer |
| CSF-2R | Brain malignant glioma |
| CSF-2R | Refractory lymphoma |
| CSF-2R | Refractory anaplastic large |
| | cell lymphoma |
| CSF-2R | Refractory T-cell lymphoma |
| CSF-2R | Merkel cell carcinoma |
| CSF-2R | Helical adenocarcinoma |
| CSF-2R | Lymphoma |
| CSF-2R | Locally advanced soft tissue |
| | sarcoma |
| CSF-2R | Locally advanced bladder |
| | cancer |
| CSF-2R | Invasive breast cancer |
| CSF-2R | Colorectal cancer liver |
| | metastasis |
| CSF-2R | Colorectal cancer |
| CSF-2R | Keratoacanthoma |
| CSF-2R | Glioblastoma |
| CSF-2R | Mesothelioma |
| CSF-2R | Basal cell carcinoma |
| PTEN | Acute spinal cord injury |
| PTH | Postmenopausal |
| | osteoporosis |
| PTK7 | Colorectal cancer |
| PTP1B | obesity |
| PTP1B | Type 2 diabetes |
| PYK2 | Ovarian cancer |
| PYK2 | Colorectal cancer |
| PYK2 | Thyroid cancer |
| Raf kinase | Head and neck tumor |
| Raf kinase | Breast cancer |
| RAG-1 | Glioblastoma |
| RANGRF | Brugada syndrome |
| Ras | Tumor |
| Ras | Tumor |
| Ras | Pancreatic acinar carcinoma |
| Ras | Secondary malignant tumor |
| | of the pancreas |
| Ras | Advanced colorectal |
| | adenocarcinoma |
| Ras | Solid tumor |
| Ras | Breast cancer |
| Ras | Locally advanced pancreatic |
| | adenocarcinoma |
| Ras | Colorectal cancer |
| Ras | Recurrent colorectal cancer |
| Ras | Non-small cell lung cancer |
| RB1 | Pancreatic cancer |
| RB1 | Head and neck tumor |
| RB1 | Mesothelioma |
| RB1 | Lung cancer |
| RDH12 | Leber congenital amaurosis |
| RdRp | Novel Coronavirus Infection |
| Redd1 | Diabetic macular edema |
| Redd1 | Macular degeneration |
| regnase-1 | Fibrosis |
| regnase-1 | Adult respiratory distress |
| | syndrome |
| CSF-2R | Basal squamous cell |
| | carcinoma |
| CSF-2R | Muscle-Invasive Bladder |
| | Cancer |
| CSF-2R | Rhabdomyosarcoma |
| CSF-2R | Melanoma |
| CSF-2R | Sweat gland tumor |
| CSF-2R | Hepatocellular carcinoma |
| CSF-2R | Liver cancer |
| CSF-2R | Recurrent breast cancer |
| CSF-2R | Relapsed T-cell lymphoma |
| CSF-2R | Recurrent HER2-negative |
| | breast cancer |
| CSF-2R | Relapsed peripheral T-cell |
| | lymphoma |
| CSF-2R | Non-muscle invasive bladder |
| | tumor |
| CSF-2R | Malignant cylindrical tumor |
| CSF-2R | Malignant solid tumor |
| CSF-2R | Ductal carcinoma |
| CSF-2R | Unresectable melanoma |
| CSF-2R | Bladder metastases |
| CSF-2R | Bladder cancer |
| CSF-2R | NK cell lymphoma |
| CSF-2R, | Tumor |
| CTLA4 | |
| CSF-2R, | Microsatellite stable |
| CTLA4, | colorectal cancer |
| GALV-GP R- | |
| CSF-2R, | Solid tumor |
| CTLA4, | |
| GALV-GP R- | |
| CSF-2R, | Solid tumor |
| GALV-GP R- | |
| CSF-2R, | Triple-negative breast cancer |
| GALV-GP R- | |
| CSF-2R, | Squamous cell carcinoma of |
| GALV-GP R- | the skin |
| CSF-2R, | Skin melanoma |
| GALV-GP R- | |
| RELA | Rheumatoid arthritis |
| RELA | Osteoarthritis |
| renin | Diabetic retinopathy |
| receptor | |
| renin | Uveitis |
| receptor | |
| renin | Macular degeneration |
| receptor | |
| REP1 | Choroideremia |
| REP1 | Retinal dysplasia |
| REP1 | X-linked retinitis |
| | pigmentosa |
| REV | HIV infection |
| RHO | Retinitis pigmentosa type 4 |
| RHO | Retinitis pigmentosa |
| RHO | Color vision impairment |
| RHO | Macular degeneration |
| RHO | Geographic atrophy |
| RHOK | Retinal disease |
| RIG-I | Tumor |
| RIG-I | Melanoma |
| RIG-I | Coronavirus infection |
| RIG-I, | Viral infection |
| TLR3 | |
| RLBP1 | Retinitis pigmentosa |
| RNAP | Hepatitis B |
| RNA | Metastatic breast cancer |
| binding | |
| proteins | |
| RNR | Metastatic solid tumor |
| RNR | Tumor |
| RNR | Rectal cancer |
| RNR | Primitive neuroectodermal |
| | tumor |
| CSF-2R, | Melanoma |
| GALV-GP R- | |
| CSF-2R, | Non-small cell lung cancer |
| GALV-GP R- | |
| CSF-2R, | MSI-H cancer |
| GALV-GP R- | |
| CSF-2R, | Solid tumor |
| PDL1 | |
| CSF-3R | Neutropenia |
| CSF-3R | Cytokine Release Syndrome |
| CSF-3R | Knee arthritis |
| CSF-3R | Advanced solid malignant |
| | tumor |
| CSF-3R | Community-acquired |
| | pneumonia |
| CSF-3R | Sepsis |
| CSF-3R | Urinary tract infection |
| CSF-3R | Mesothelioma |
| CSF-3R | Acute cholecystitis |
| CSF-3R | Abdominal infection |
| CSF-3R | Secondary malignant tumor |
| | of peritoneum |
| CSF-3R | Peritoneal cancer |
| CSF-3R | cholangitis |
| CTCFL | Breast cancer |
| CTGF | Head and neck tumor |
| CTGF | Idiopathic pulmonary fibrosis |
| CTGF | Hypertrophic scars of the |
| | skin |
| CTGF | Ovarian cancer |
| CTGF | Melanoma |
| CTGF | Cirrhosis |
| CTGF | Trauma and Injury |
| CTGF | Scar |
| CTGF, | Duchenne muscular |
| NF-κB | dystrophy |
| RNR | Solid tumor |
| RNR | Kidney tumor |
| RNR | Renal cell carcinoma |
| RNR | Breast cancer |
| RNR | Prostate cancer |
| RNR | Prostate cancer |
| RNR | Refractory undifferentiated |
| | acute leukemia |
| RNR | Supratentorial tumor |
| RNR | Chronic myeloid leukemia |
| RNR | Lymphoma |
| RNR | Locally advanced non-small |
| | cell lung cancer |
| RNR | Colorectal cancer |
| RNR | Glioblastoma |
| RNR | Secondary myelodysplastic |
| | syndrome |
| RNR | Chronic myeloid leukemia |
| | in acute transformation |
| RNR | Acute promyelocytic |
| | leukemia |
| RNR | Acute leukemia |
| RNR | Myelodysplastic syndrome |
| RNR | Recurrent prostate cancer |
| RNR | Recurrent colon cancer |
| RNR | Relapsed acute myeloid |
| | leukemia |
| RNR | Recurrent non-small cell |
| | lung cancer |
| RNR | Relapsed adult acute |
| | lymphoblastic leukemia |
| RNR | Non-small cell lung cancer |
| | stage IIIB |
| RNR | Non-small cell lung cancer |
| | stage IIIA |
| RNR | Non-small cell lung cancer |
| RNR | Acute myeloid leukemia in |
| | adults |
| CTLA4 | Tumor |
| CTLA4 | Sjögren's syndrome |
| CTLA4, | Solid tumor |
| FLT3, | |
| IL-12R | |
| CTLA4, | Metastatic non-small cell |
| IL-12 | lung cancer |
| CTLA4, | Solid tumor |
| MSLN, PD-1 | |
| CTLA4, | Advanced solid malignant |
| PD-1 | tumor |
| CTLA4, | Colon cancer |
| PD-1 | |
| CTLA4, | Tumor |
| STAT3 | |
| CTNNB1 | Tumor |
| CTNNB1 | Type 2 diabetes |
| CTNS | Cystinosis |
| CXCR2, | Liver cancer |
| GPC3 | |
| CXCR3, | Tumor |
| IL-12, TGF-β | |
| CXCR4 | Primary myelofibrosis |
| CXCR4 | Heart failure |
| CXCR4 | Perioperative ischemia |
| CXCR4 | Peripheral arterial disease |
| CXCR4 | Surgical wound |
| CXCR4 | Myelofibrosis |
| CXCR5, | Non-small cell lung cancer |
| EGFR | |
| CYB5R3 | Tumor |
| CYBB | Chronic granulomatous |
| | disease |
| Cyclin G1 | Pancreatic cancer |
| Cyclin G1 | Solid tumor |
| Cyclin G1 | Breast cancer |
| Cyclin G1 | Sarcoma |
| Cyclin G1 | Colorectal cancer |
| RNR | Adult acute lymphoblastic |
| | leukemia |
| RNR | Bladder cancer |
| RNR | Acute myeloid leukemia |
| | with 11q23 abnormalities |
| RNR | IV malignant non-small cell |
| | lung tumor |
| ROBO1 | Tumor |
| ROBO1 | Pancreatic cancer |
| ROBO1 | Solid tumor |
| ROCK | Tumor |
| ROCK | Retinitis pigmentosa |
| ROCK | Corneal edema |
| ROCK | Corneal endotheliitis |
| ROCK | Bullous keratopathy |
| ROCK | Fuchs endothelial dystrophy |
| ROR1 | Metastatic non-small cell |
| | lung cancer |
| ROR1 | Hematologic malignancy |
| ROR1 | Advanced breast cancer |
| ROR1 | Mantle cell lymphoma |
| ROR1 | Solid tumor |
| ROR1 | Triple-negative breast |
| | cancer |
| ROR1 | Breast cancer |
| ROR1 | Breast cancer |
| ROR1 | Refractory malignant solid |
| | tumor |
| ROR1 | Diffuse large B-cell |
| | lymphoma |
| ROR1 | Chronic lymphocytic |
| | leukemia |
| ROR1 | Ovarian cancer |
| ROR1 | Acute lymphoblastic |
| | leukemia |
| ROR1 | Recurrent breast cancer |
| Cyclin G1 | Osteosarcoma |
| CYP21A2 | Congenital adrenal |
| | hyperplasia |
| CYP27A1 | Cerebrotendinous |
| | xanthomatosis |
| CYP3A4 | Tumor |
| CYP4V2 | Retinal degeneration |
| CYP4V2 | Bietti crystal dystrophy |
| CYPs | Pancreatic cancer |
| CYPs | Colorectal cancer |
| Cytokines | Sarcoma |
| Cytokines | Melanoma |
| Cytokines | Malignant solid tumor |
| C-type lectin | Testicular tumor |
| domain | |
| protein | |
| DAF | Macular degeneration |
| DAF, | Solid tumor |
| ICAM-1 | |
| DAF, | Non-small cell lung cancer |
| ICAM-1 | |
| DAG1 | Muscular dystrophy |
| DAG1 | Duchenne muscular |
| | dystrophy |
| DAT | Parkinson's disease |
| DDC | Parkinson's disease |
| DDC | Aromatic amino acid |
| | decarboxylase deficiency |
| DGAT2 | Nonalcoholic steatohepatitis |
| DGAT2 | Type 2 diabetes |
| DGKK | Fragile X syndrome |
| DKK3 | Mesothelioma |
| DKK3 | Malignant pleural |
| | mesothelioma |
| DLL3 | Recurrence of small cell lung |
| | cancer |
| DLL3 | Small cell lung cancer |
| DLL3 | Large cell neuroendocrine |
| | carcinoma |
| DMD exon | Muscular dystrophy |
| 055 | |
| ROR1 | Recurrent non-small cell |
| | lung cancer |
| ROR1 | Non-Hodgkin lymphoma |
| ROR1 | Leukemia |
| ROR1 | B-cell malignancy |
| ROR2 | Pancreatic cancer |
| ROR2 | Gastric cancer |
| ROR2 | Kidney tumor |
| ROR2 | Renal cell carcinoma |
| ROR2 | Sarcoma |
| ROR2 | Recurrent solid tumor |
| ROR2 | Bladder cancer |
| RORG | Immune system disorders |
| RORα | Stargardt disease |
| RORα | Retinitis pigmentosa type 19 |
| RORα | Dry age-related macular |
| | degeneration |
| RORα | Geographic atrophy |
| RORα | Cone-rod dystrophy type 3 |
| RPE65 | Leber congenital amaurosis |
| RPE65 | Leber's hereditary optic |
| | atrophy |
| RPGR | Choroideremia |
| RPGR | Retinoschisis |
| RPGR | X-linked retinitis |
| | pigmentosa |
| RPN2 | Breast cancer |
| RRM2 | Tumor |
| RRM2 | Solid tumor |
| RT | Lymphoma |
| RT | HIV infection |
| RTK | Ovarian cancer |
| RUNX3 | Tumor |
| DMD exon | Muscular dystrophy |
| 44 | |
| DMD exon | Duchenne muscular |
| 44 | dystrophy |
| DMD exon | Duchenne muscular |
| 45 | dystrophy |
| DMD exon | Duchenne muscular |
| 45, DMD | dystrophy |
| exon 44 | |
| DMD exon | Duchenne muscular |
| 50 | dystrophy |
| DMD exon | Duchenne muscular |
| 51 | dystrophy |
| DMD exon | Muscular dystrophy |
| 52 | |
| DMD exon | Duchenne muscular |
| 52 | dystrophy |
| DMD exon | Duchenne muscular |
| 53 | dystrophy |
| DMD exon | Duchenne muscular |
| 53 | dystrophy |
| DMD exon | Duchenne muscular |
| 55 | dystrophy |
| Dm-dNK | Breast cancer |
| Dm-dNK | Colorectal cancer |
| DMPK | Myotonic dystrophy |
| DNA | Pancreatic cancer |
| DNA | Cytomegalovirus infection |
| polymerase, | |
| RT | |
| DNA | Herpes simplex |
| polymerase, | |
| RT | |
| DNA | Epstein-Barr virus infection |
| polymerase, | |
| RT | |
| RYR2 | Catecholaminergic |
| | polymorphic ventricular |
| | tachycardia |
| SANS | Usher syndrome |
| SARM1 | Peripheral nervous system |
| | disease |
| SARS-CoV-2 | Novel Coronavirus Infection |
| 3CLpro | |
| SARS-CoV-2 | Novel Coronavirus Infection |
| N protein, | |
| SARS-CoV-2 | |
| ORF1Ab | |
| SARS-CoV-2 | Novel Coronavirus Infection |
| S protein | |
| SARS-CoV-2 | Coronavirus infection |
| S protein | |
| SATB1 | Prostate cancer |
| SCAP | Dyslipidemia |
| SCAP | Metabolic fatty liver disease |
| SDF-1 | Hematopoietic stem cell |
| | transplantation |
| SDF-1 | Pancreatic cancer |
| SDF-1 | Heart damage |
| SDF-1 | Diabetic retinopathy |
| SDF-1 | Chronic lymphocytic |
| | leukemia |
| SDF-1 | Colorectal cancer |
| SDF-1 | Relapsed chronic |
| | lymphocytic leukemia |
| SDF-1 | Multiple myeloma |
| DNA | HIV infection |
| polymerase, | |
| RT | |
| DNA | Tumor |
| polymerase, | |
| thymidine | |
| kinase | |
| DNA-directed | Autoimmune disease |
| DNA | |
| polymerase | |
| DNA-directed | Tumor |
| DNA | |
| polymerase | |
| DNA-directed | Inflammation |
| DNA | |
| polymerase | |
| DNA-directed | Influenza A virus infection |
| DNA | |
| polymerase | |
| DNA-directed | Dystonia |
| DNA | |
| polymerase | |
| DNAJC15 | Acute kidney injury |
| DNAJC15 | Polycystic kidney disease |
| DNase I | Pancreatic acinar carcinoma |
| DNase I | Colorectal cancer |
| DNA repair | Advanced cancer |
| proteins | |
| DNA repair | Melanoma |
| proteins | |
| DNA-directed | Bacterial infection |
| RNA | |
| polymerase | |
| DNM2 | Muscle atrophy |
| DNMT1 | Head and neck tumor |
| DNMT1 | Kidney tumor |
| DNMT1 | Renal cell carcinoma |
| DNMT1 | Acute myeloid leukemia |
| DNMT1 | Myelodysplastic syndrome |
| DOK7 | Motor neurone disease |
| Selectins | Inflammation |
| Selectins | Colon cancer |
| Selectins | Melanoma |
| SEMA4A | Multiple myeloma |
| SERCA2 | Heart failure |
| SERCA2 | Diastolic heart failure |
| SERCA2 | Heart failure with preserved |
| | ejection fraction |
| SERCA2 | Ischemic cardiomyopathy |
| SERCA2 | Urinary incontinence |
| SERCA2 | Dilated cardiomyopathy |
| SERCA2 | Pulmonary hypertension |
| SERCA2 | Nonischemic |
| | cardiomyopathy |
| SERCA2 | Duchenne muscular |
| | dystrophy |
| SERCA2 | DMD-related dilated |
| | cardiomyopathy |
| SGCA | Limb-girdle muscular |
| | dystrophy |
| SGCA | Sarcoglycanosis |
| SGCB | Limb-girdle muscular |
| | dystrophy |
| SGCB | 2e type of muscular |
| | dystrophy |
| SGCG | Limb-girdle muscular |
| | dystrophy R5 |
| SGCG | Limb-girdle muscular |
| | dystrophy type 2C |
| SGLT2 | Type 2 diabetes |
| DOK7 | Congenital myasthenic |
| | syndrome |
| DR4, TRAIL | Kidney tumor |
| DR5 | Liver cancer |
| DRDs | Parkinson's disease |
| DSG1, DSG3 | Pemphigus |
| DSG3 | Pemphigus |
| DSG3 | Familial pemphigus vulgaris |
| DUSP1 | Heart failure |
| DUSP1 | Arrhythmias |
| DUSP1 | Heart failure NYHA class III |
| DUSP1 | Myocardial ischemia |
| DUSP1 | Diastolic heart failure |
| DUSP1 | Systolic heart failure |
| DUSP1 | Complication |
| DUX4 | Facioscapulohumeral |
| | muscular dystrophy |
| DUX4 | Facioscapulohumeral |
| | muscular dystrophy type 1a |
| DWORF | Heart failure with reduced |
| | ejection fraction |
| DWORF | Dilated cardiomyopathy |
| DYSF | Limb-girdle muscular |
| | dystrophy |
| DYSF | Miyoshi myopathy |
| DYSF | Myofibrillar myopathy |
| DYSF | Dysferlin myopathy |
| DYSF | Limb-Girdle Muscular |
| | Dystrophy Type 2B |
| Dystrophin | Angelman syndrome |
| Dystrophin | Muscular dystrophy |
| Dystrophin | Duchenne muscular |
| | dystrophy |
| E2F1 | End-stage renal disease |
| E2F1 | Hyperplasia |
| SGSH | Mucopolysaccharidosis type |
| | III |
| SGSH, | Mucopolysaccharidosis type |
| SUMF1 | III |
| SHANK3 | Autism Spectrum Disorders |
| SHANK3 | Phelan-McDermid |
| | syndrome |
| SIRPα | Tumor |
| SIRT6 | Nonalcoholic steatohepatitis |
| SLAMF7 | Hematologic malignancy |
| SLAMF7 | Multiple myeloma |
| SLC6A8 | Duchenne muscular |
| | dystrophy |
| SMAC | Pancreatic cancer |
| SMAC, | Hepatocellular carcinoma |
| TRAIL | |
| SMAD2, | Choroidal |
| TGFBR2, | neovascularization |
| VEGF-A | |
| SMAD4 | Colorectal cancer |
| SMAD7 | Crohn's disease |
| SMAD7 | Cachexia |
| SMAD7 | Duchenne muscular |
| | dystrophy |
| SMAD7 | Inclusion body myositis |
| SMN1 | Spinal muscular atrophy |
| SMN1 | Muscular dystrophy |
| SMN1 | Spinal muscular atrophy |
| | type I |
| SMN2 | Infantile-onset |
| | spinocerebellar ataxia |
| SMN2 | Spinal muscular atrophy |
| SMN2 exon | Spinal muscular atrophy |
| 7 | |
| SMPD1 | Niemann-Pick disease |
| SNAP25 | Primary axillary |
| | hyperhidrosis |
| SOCS1 | Advanced solid malignant |
| | tumor |
| SOCS1 | Head and neck tumor |
| SOCS1 | Head and Neck Squamous |
| | Cell Carcinoma |
| E2F1 | Vascular graft obstruction |
| E2F1 | Perioperative ischemia |
| E2F1 | Peripheral vascular disease |
| E2F1 | Coronary artery restenosis |
| E2F1 | Coronary heart disease |
| E2F1 | Arterial occlusive disease |
| E6 | Penile tumor |
| E6 | Vaginal tumor |
| E6 | Vulvar tumor |
| E6 | Papillomavirus infection |
| E6 | Squamous intraepithelial |
| | lesion |
| E6 | Oropharyngeal tumor |
| E6 | Cervical cancer |
| E6 | Cervical cancer |
| E6 | Anal tumor |
| E6 | Polyomavirus infection |
| E6 | HPV16 infection |
| E6, HPV E7 | Head and neck tumor |
| E6, HPV E7 | Cervical cancer |
| E6, PD-1 | Metastatic cervical cancer |
| E6, PD-1 | Cervical cancer |
| EBNA1, | Hodgkin lymphoma |
| LMP1, | |
| BARF1 | |
| proteins | |
| EBNA1, | Non-Hodgkin lymphoma |
| LMP1, | |
| BARF1 | |
| proteins | |
| EBV proteins | Hematopoietic stem cell |
| | transplantation |
| EBV proteins | Transplant complications |
| EBV proteins | Hematologic malignancy |
| EBV proteins | Gastric cancer |
| EBV proteins | Leiomyosarcoma |
| EBV proteins | Nasopharyngeal carcinoma |
| SOCS1 | Melanoma |
| SOCS1 | Lung cancer |
| SOCS1 | Non-small cell lung cancer |
| SOCS3 | Hepatocellular carcinoma |
| SOD1 | Motor neurone disease |
| SOD1 | Motor neurone disease |
| SOD1 | Amyotrophic lateral |
| | sclerosis |
| SOD1 | Amyotrophic lateral |
| | sclerosis type 1 |
| SOS1 | Lung cancer |
| SOST | Tumor |
| SOST | Triple-negative breast |
| | cancer |
| SOST | Familial hypophosphatemic |
| | rickets |
| SOST | Osteoporosis |
| SOST | Osteogenesis imperfecta |
| SOST | Type 2 diabetes |
| SPAG9 | Prostate cancer |
| SPAT | Primary hyperoxaluria |
| SPHK1 | Hepatocellular carcinoma |
| SPSB1 | Non-small cell lung cancer |
| SRY | Parkinson's disease |
| SSTR, | Ovarian cancer |
| thymidine | |
| kinase | |
| SSTR, | Recurrent ovarian cancer |
| thymidine | |
| kinase | |
| SSTR2 | Melanoma |
| ST13 | Colorectal cancer |
| ST13, | Pancreatic ductal carcinoma |
| TRAIL | |
| STAT1 | Asthma |
| STAT3 | Metastatic head and neck |
| | squamous cell carcinoma |
| STAT3 | Hereditary nonpolyposis |
| | colorectal cancer type 1 |
| STAT3 | Pancreatic acinar carcinoma |
| EBV proteins | Epstein-Barr virus-positive |
| | lymphoma |
| EBV proteins | Epstein-Barr virus-associated |
| | lymphoproliferative disease |
| EBV proteins | Epstein-Barr virus-associated |
| | nasopharyngeal carcinoma |
| EBV proteins | Ebola virus disease |
| EBV proteins | EBV-related post-transplant |
| | lymphoproliferative disorder |
| EGF | HER2-positive breast cancer |
| EGFR | Metastatic head and neck |
| | squamous cell carcinoma |
| EGFR | Ewing sarcoma |
| EGFR | Advanced solid malignant |
| | tumor |
| EGFR | Clear cell sarcoma |
| EGFR | Head and neck tumor |
| EGFR | Retinoblastoma |
| EGFR | Solid tumor |
| EGFR | Wilms tumor |
| EGFR | Neurofibrosarcoma |
| EGFR | Neuroblastoma |
| EGFR | Triple-negative breast cancer |
| EGFR | Squamous cell carcinoma |
| EGFR | Glioblastoma |
| EGFR | Synovial sarcoma |
| EGFR | Rhabdoid tumor |
| EGFR | Rhabdomyosarcoma |
| EGFR | Hepatocellular carcinoma |
| EGFR | Hepatoblastoma |
| EGFR | Recurrent solid tumor |
| EGFR | Non-small cell lung cancer |
| EGFR | Malignant epithelial tumor |
| EGFR | Desmoplastic small round |
| | cell tumor |
| EGFR | Trauma and Injury |
| STAT3 | Advanced colorectal |
| | adenocarcinoma |
| STAT3 | Advanced non-small cell |
| | lung cancer |
| STAT3 | Advanced solid malignant |
| | tumor |
| STAT3 | Head and neck tumor |
| STAT3 | Kidney tumor |
| STAT3 | Adrenoleukodystrophy |
| STAT3 | Breast cancer |
| STAT3 | Prostate cancer metastasis |
| STAT3 | Refractory colorectal cancer |
| STAT3 | Refractory lung cancer |
| STAT3 | Ovarian cysts |
| STAT3 | Glioma |
| STAT3 | Acute myeloid leukemia |
| STAT3 | Myelodysplastic syndrome |
| STAT3 | Hepatocellular carcinoma |
| STAT3 | Recurrent squamous cell |
| | carcinoma of the head and |
| | neck |
| STAT3 | Non-small cell lung cancer |
| STAT3 | Bladder cancer |
| STAT3 | MSI-H Colorectal Cancer |
| STAT3, | Pancreatic cancer |
| mTOR | |
| STAT3, | Bladder cancer |
| mTOR | |
| STAT3, | Tumor |
| TLR9 | |
| STAT3, | Acute myeloid leukemia |
| TLR9 | |
| STAT3, | B-cell lymphoma |
| TLR9 | |
| STAT5 | Breast cancer |
| transcription | |
| factor | |
| STAT6 | Pancreatic acinar carcinoma |
| STAT6 | Gastric cancer |
| STAT6 | Advanced hepatocellular |
| | carcinoma |
| STAT6 | Breast cancer |
| EGFR | HER2-positive breast cancer |
| EGFR | EGFR mutant solid tumor |
| EGFR | EGFR mutant solid tumor |
| EGFR, c-Met | Nonalcoholic steatohepatitis |
| EGFR, | Glioblastoma |
| EGFRvIII | |
| EGFR, | Recurrent glioblastoma |
| EGFRVIII, | |
| HER2, | |
| IL-13Rα2 | |
| EGFR, IL-12 | Colorectal cancer |
| EGFR, | Glioblastoma |
| IL-13Rα2 | |
| EGFR, | Recurrent glioblastoma |
| IL-13Rα2 | |
| EGFR, | EGFR-mutated glioblastoma |
| IL-13Rα2 | |
| EGFR, | Leukemia |
| IL-21R | |
| EGFR, SSTR | Pancreatic cancer |
| EGFR, SSTR | Head and Neck Squamous |
| | Cell Carcinoma |
| EGFR, SSTR | Breast cancer |
| EGFR, SSTR | Colorectal cancer |
| EGFR, SSTR | Melanoma |
| EGFR, SSTR | Liver cancer |
| EGFR, | Pancreatic ductal |
| TCRDV2 | adenocarcinoma |
| EGFR, | Head and Neck Squamous |
| TCRDV2 | Cell Carcinoma |
| EGFR, | Solid tumor |
| TCRDV2 | |
| EGFR, | Colorectal cancer |
| TCRDV2 | |
| EGFR, | Non-small cell lung cancer |
| TCRDV2 | |
| EGFR, | Tumor |
| TGF-β | |
| EGFRvIII | Meningeal tumor |
| EGFRvIII | Glioblastoma |
| EGFRvIII | Liver cancer |
| STAT6 | Uveal melanoma |
| STAT6 | Ovarian epithelial |
| | carcinoma |
| STAT6 | Colorectal cancer |
| STAT6 | Glioma |
| STAT6 | Thyroid cancer |
| STAT6 | Liver metastasis |
| STAT6 | Liver cancer |
| STAT6 | Lung cancer |
| STEAP1 | Metastatic |
| | castration-resistant prostate |
| | cancer |
| STEAP1 | Prostate cancer metastasis |
| STEAP2, | Prostate cancer metastasis |
| TGFBR2 | |
| STEAP2, | Prostate cancer |
| TGFBR2 | |
| STING | Liver cancer |
| STMN1 | Solid tumor |
| STMN2 | Amyotrophic lateral |
| | sclerosis |
| STMN2 | Frontotemporal dementia |
| STRC | Hearing impairment |
| STRIP1 | Solid tumor |
| STRIP1 | Glioma |
| STXBP1 | STXBP1-related epileptic |
| | encephalopathy |
| SULF2, | Pancreatic acinar carcinoma |
| TGF-β1 | |
| SULF2, | Liver cancer |
| TGF-β1 | |
| SULF2, | Lung cancer |
| TGF-β1 | |
| SUMO1 | Heart failure |
| SURF1 | Leigh disease |
| Survivin | Metastatic non-small cell |
| | lung cancer |
| EGFRvIII | Lung cancer |
| EGFRvIII | Glioblastoma multiforme |
| EGFRvIII, | Glioblastoma |
| EphA2, | |
| HER2, | |
| IL-13Rα2 | |
| EGR1 | Tumor |
| EGR1 | Postoperative pain |
| EHMT2 | Prader-Willi syndrome |
| EIF2AK4 | Tumor |
| EIF2AK4 | Immune system disorders |
| EIF4E | Tumor |
| EIF4E | Advanced cancer |
| EIF4E | Castration-resistant prostate |
| | cancer |
| EIF4E | Prostate cancer |
| EIF4E | Non-small cell lung cancer |
| ELA2 | Tumor |
| ELA2 | Duchenne muscular |
| | dystrophy |
| elastin | Skin disorders |
| ELAVL1 | Pancreatic cancer |
| ELAVL1 | Ovarian cancer |
| ELF3 | Tuberculosis |
| ELOVL2 | Dry age-related macular |
| | degeneration |
| ELP1, | Familial dysautonomia |
| splicing | |
| factor | |
| EMR1 | Leukemia |
| ENaC | Cystic fibrosis |
| enkephalinase | B-cell leukemia |
| eNOS | Myocardial infarction |
| eNOS | Peripheral vascular disease |
| eNOS | Pulmonary hypertension |
| env | Chronic hepatitis B |
| Survivin | Tumor |
| Survivin | Advanced hepatocellular |
| | carcinoma |
| Survivin | Solid tumor |
| Survivin | Castration-resistant prostate |
| | cancer |
| Survivin | Precursor T-lymphocytic |
| | leukemia lymphoma |
| Survivin | Prostate cancer |
| Survivin | Lymphoma |
| Survivin | Locally advanced non-small |
| | cell lung cancer |
| Survivin | Glioblastoma |
| Survivin | Acute myeloid leukemia |
| Survivin | Acute lymphoblastic |
| | leukemia |
| Survivin | Hepatocellular carcinoma |
| Survivin | Relapsed adult acute |
| | lymphoblastic leukemia |
| Survivin | Non-small cell lung cancer |
| SYF2 | Amyotrophic lateral |
| | sclerosis |
| SYF2 | Frontotemporal dementia |
| Syk | Inflammation |
| Syk | Asthma |
| SYT11 | Gastric cancer |
| SYT11 | Colorectal cancer |
| SYT11 | Lung cancer |
| TAG-72 | Ovarian cancer |
| TAG-72 | Platinum-resistant Ovarian |
| | epithelial carcinoma |
| TAG-72 | T-cell lymphoma |
| TAU | Mild cognitive impairment |
| TAU | Pick's disease dementia |
| TAU | Progressive supranuclear |
| | palsy |
| TAU | Dementia due to |
| | Alzheimer's disease |
| EpCAM | Digestive system tumor |
| EpCAM | Gastric cancer |
| EpCAM | Advanced Gastric Cancer |
| EpCAM | Solid tumor |
| EpCAM | Liver cancer |
| EphA2 | Pancreatic cancer |
| EphA2 | Solid tumor |
| EphA2 | Ovarian cancer |
| EphA2 | Lymphoma |
| EphA2 | Locally advanced malignant |
| | solid tumor |
| EphA2 | Glioblastoma |
| EphA2 | Glioma |
| EphA2 | Lung cancer |
| EphA2, | EGFR-mutated glioblastoma |
| IL-13Rα2 | |
| EphA3 | Solid tumor |
| EPO receptor | End-stage renal disease |
| EPO receptor | Renal anemia |
| EPO receptor | Neurodegenerative disease |
| EPO receptor | Glaucoma |
| EPO receptor | Anemia |
| EPO receptor | Parkinson's disease |
| ERVE-1 | Kidney tumor |
| ERα | Tumor |
| E-sel | Thrombotic occlusive |
| | vasculitis |
| E-sel | Vascular disease |
| E-sel | Peripheral arterial disease |
| EWS-FLI1 | Ewing sarcoma |
| EZH2 | Solid tumor |
| F10 | Hemophilia A |
| F10, F8 | Hemophilia A |
| F11 | End-stage renal disease |
| F11 | Thromboembolism |
| F11 | Cardiovascular disease |
| TAU | Alzheimer's disease |
| TAU, | Neurodegenerative disease |
| α-synuclein | |
| TBX21 | Psoriasis |
| TBX21 | Chronic obstructive |
| | pulmonary disease |
| TBXA2R | Tumor |
| TBXA2R | Cardiovascular disease |
| TCF7L2 | Tumor |
| TCR | B-cell lymphoma |
| TCR, | Neuroblastoma |
| VCAM1 | |
| TDP43 | Amyotrophic lateral |
| | sclerosis |
| TDP43 | Frontotemporal dementia |
| TECPR2 | Spastic paraplegia |
| Telomerase | Gastroesophageal junction |
| | adenocarcinoma |
| Telomerase | Gastric cancer |
| Telomerase | Perioperative ischemia |
| Telomerase | Esophageal cancer |
| Telomerase | Glioblastoma |
| Telomerase | Melanoma |
| Telomerase | Liver cancer |
| Telomerase | Non-small cell lung cancer |
| TERC | Neutropenia |
| TERC | Juvenile myelomonocytic |
| | leukemia |
| TERC | Transfusion-dependent |
| | anemia |
| TERC | Acute myeloid leukemia |
| TERC | Myelodysplastic syndrome |
| TERC | Myelofibrosis |
| TERT | Advanced non-small cell |
| | lung cancer |
| TERT | Head and neck tumor |
| TERT | Glioblastoma |
| TERT | Melanoma |
| TERT | Hepatocellular carcinoma |
| TERT | Non-small cell lung cancer |
| TERT, | Tumor |
| c-Myc | |
| F11 | Myocardial infarction |
| F11 | Upper extremity deep vein |
| | thrombosis |
| F11 | Coagulation disorders |
| F11 | Stroke |
| F11 | Venous thrombosis |
| F11 | Arterial thrombosis |
| F11R | Androgenic Alopecia |
| F12 | Thromboembolism |
| F12 | Cardiovascular disease |
| F12 | Metabolic disease |
| F8 | Hemophilia A |
| F8 | Antineutrophil cytoplasmic |
| | antibody-associated vasculitis |
| F8 | X-linked genetic disease |
| F8, factor IX | Hemophilia B |
| F8, factor IX | Hemophilia A |
| factor IX | Hemophilia B |
| factor IX | Venous thrombosis |
| factor IX | Acute Coronary Syndrome |
| factor IX | Acute Coronary Syndrome |
| factor IX | Coronary heart disease |
| factor IX | Nonalcoholic steatohepatitis |
| Factor VII | Thrombosis |
| Factor VII | Factor VII deficiency |
| FANCA | Tyrosinemia |
| FANCA | Fanconi anemia |
| FANCC | Fanconi anemia |
| FAP | Tumor |
| FAP, PDL1 | Solid tumor |
| FasL | Sarcoma |
| FasL | Lymphoma |
| FasL | Malignant epithelial tumor |
| FASN, | Fallopian tube cancer |
| MSLN | |
| TERT, | Tumor |
| αvβ3, αvβ5 | |
| TERT, | Tumor |
| αvβ5 | |
| TFDP3 | Tumor |
| TFPI | Hemophilia B |
| TFPI | Hemophilia A |
| TFPI | hemophilia |
| TfR1 | Neuromuscular disorders |
| TfR1 | Duchenne muscular |
| | dystrophy |
| TG | Brain metastases |
| TGFBI | Hereditary corneal |
| | dystrophy |
| TGFBR2 | Tumor |
| TGFBR2 | Pulmonary fibrosis |
| TGF-β | Heart failure |
| TGF-β | Gastric cancer |
| TGF-β | Solid tumor |
| TGF-β | Chondrosarcoma |
| TGF-β | Relapsed plasma cell |
| | myeloma |
| TGF-β | Multiple myeloma |
| TGF-β | Infectious mononucleosis |
| TGF-β | Nasopharyngeal carcinoma |
| TGF-β | Niemann-Pick disease type |
| | C |
| TGF-β1 | Novel Coronavirus Infection |
| TGF-β1 | Idiopathic pulmonary |
| | fibrosis |
| TGF-β1 | Corneal disease |
| TGF-β1 | Basal cell carcinoma |
| TGF-β1 | Osteoarthritis |
| TGF-β1 | Liver cancer |
| TGF-β1, | Tumor |
| VEGFR2 | |
| TGF-β1, | Joint disease |
| α-Klotho | |
| TGF-β2 | Metastatic solid tumor |
| TGF-β2 | Metastatic non-small cell |
| | lung cancer |
| TGF-β2 | Tumor |
| FASN, | Ovarian cancer |
| MSLN | |
| FASN, | Peritoneal cancer |
| MSLN | |
| FasR | Hepatitis |
| FasR | Liver failure |
| FasR, TNFR1 | Metastatic colorectal cancer |
| FasR, TNFR1 | Colorectal cancer liver |
| | metastasis |
| FasR, TNFR1 | Recurrent platinum-resistant |
| | ovarian cancer |
| FasR, TNFR1 | Glioblastoma multiforme |
| FGF19 | Nonalcoholic steatohepatitis |
| FGF21 | Heart failure |
| FGF21 | Myocardial disease |
| FGF21 | Familial partial lipid |
| | dystrophy |
| FGF21 | obesity |
| FGF21 | Metabolic fatty liver disease |
| FGF21, | Nonalcoholic steatohepatitis |
| FGF21R | |
| FGF21, | Heart disease |
| TGF-β1 | |
| FGF21, | Senescence |
| TGF-β1 | |
| FGF21, | Kidney disease |
| TGF-β1 | |
| FGF21, | Metabolic disease |
| TGF-β1 | |
| FGF4 | Stable angina |
| FGF4 | Refractory angina |
| FGF4 | Coronary heart disease |
| FGFR3 | Chondrodysplasia |
| FGFR4 | Rhabdomyosarcoma |
| FGFR4 | Hepatocellular carcinoma |
| FGFR4 | Obesity |
| FGFRs | Hair loss |
| TGF-β2 | Bronchogenic carcinoma |
| TGF-β2 | Pancreatic cancer |
| TGF-β2 | Advanced non-small cell |
| | lung cancer |
| TGF-β2 | Diabetic macular edema |
| TGF-β2 | Solid tumor |
| TGF-β2 | Wet macular degeneration |
| TGF-β2 | Renal cell carcinoma |
| TGF-β2 | Diffuse midline glioma |
| TGF-β2 | Diffuse intrinsic pontine |
| | glioma |
| TGF-β2 | Glioblastoma |
| TGF-β2 | Glioma |
| TGF-β2 | Melanoma |
| TGF-β2 | Non-small cell lung cancer |
| TGF-β2 | Malignant pleural |
| | mesothelioma |
| TGM1 | Autosomal recessive |
| | ichthyosis |
| TGM2 | Tumor |
| TGM2 | Immune system disorders |
| THBS1 | Pancreatic cancer |
| THBS1 | Lung cancer |
| THPO | Thrombocytopenia |
| thrombin | Thrombosis |
| thrombin | heart disease |
| Thymidine | Tumor |
| kinase | |
| Thymidine | Graft-versus-host disease |
| kinase | |
| Thymidine | Pancreatic adenocarcinoma |
| kinase | |
| Thymidine | Hematologic malignancy |
| kinase | |
| Thymidine | Gastric cancer |
| kinase | |
| FGFRs | Multiple sclerosis |
| FGL1 | Tumor |
| FKRP | Limb-girdle muscular |
| | dystrophy |
| FKRP | Miyoshi myopathy |
| FLAP | Allergic conjunctivitis |
| FLT3 | Refractory acute myeloid |
| | leukemia |
| FLT3 | Acute myeloid leukemia |
| FLT3 | Relapsed acute myeloid |
| | leukemia |
| FLT3 | Leukemia |
| FLT3 | FLT3-positive acute myeloid |
| | leukemia |
| FMRP | Fragile X syndrome |
| FOLR1 | Mucinous |
| | cystadenocarcinoma |
| FOLR1 | Primary peritoneal serous |
| | adenocarcinoma |
| FOLR1 | Undifferentiated ovarian |
| | cancer |
| FOLR1 | Advanced solid tumor |
| FOLR1 | Fallopian tube clear cell |
| | adenocarcinoma |
| FOLR1 | Fallopian tube cancer |
| FOLR1 | Renal cell carcinoma |
| FOLR1 | Urothelial carcinoma |
| FOLR1 | Urothelial carcinoma |
| FOLR1 | Myxoma |
| FOLR1 | Ovarian endometrioid |
| | carcinoma |
| FOLR1 | Ovarian epithelial carcinoma |
| FOLR1 | Ovarian epithelial carcinoma |
| Thymidine | Fallopian tube cancer |
| kinase | |
| Thymidine | Ovarian cancer |
| kinase | |
| Thymidine | Localized prostate cancer |
| kinase | |
| Thymidine | Colorectal cancer |
| kinase | |
| Thymidine | Glioma |
| kinase | |
| Thymidine | Melanoma |
| kinase | |
| Thymidine | Liver cancer |
| kinase | |
| Thymidine | Platinum-sensitive primary |
| kinase | peritoneal carcinoma |
| Thymidine | Leukemia |
| kinase | |
| Tie-2 | Diabetic retinopathy |
| TIGIT | Tumor |
| TLR | Hepatocellular carcinoma |
| TLR2 | Parkinson's disease |
| TLR3 | Breast cancer |
| TLR3 | Immune system disorders |
| TLR3 | Ovarian cancer |
| TLR4 | Stroke |
| TLR4 | Lymphatic malformations |
| TLR4 | Infect |
| TLR4 | Non-muscle invasive |
| | bladder tumor |
| TLR7 | Idiopathic pulmonary |
| | fibrosis |
| TLR7 | Non-small cell lung cancer |
| TLR7, | Tumor |
| TLR8 | |
| TLR7, | Infect |
| TLR8 | |
| FOLR1 | Ovarian mixed epithelial |
| | carcinoma |
| FOLR1 | Ovarian cancer |
| FOLR1 | Serous cystadenocarcinoma |
| FOLR1 | Osteosarcoma |
| FOLR1 | Peritoneal cancer |
| FOLR1 | Peritoneal cancer |
| FOLR1 | Recurrent primary peritoneal |
| | cancer |
| FOLR1 | Recurrent ovarian cancer |
| FOLR1 | Non-small cell lung cancer |
| FOXG1 | Neurological disorders |
| FOXO1 | Solid tumor |
| FSHR | Ovarian cancer |
| FtsZ | Acinetobacter baumannii |
| | infection |
| FUS | Amyotrophic lateral sclerosis |
| FUS | Amyotrophic lateral sclerosis |
| FXN | Friedreich's ataxia |
| G6PC1 | Glycogen storage disease |
| G6PC1 | Glycogen storage disease |
| | type I |
| GABA | Temporal lobe epilepsy |
| receptor | |
| TLR7, | psoriasis |
| TLR8, | |
| TLR9 | |
| TLR7, | Dermatomyositis |
| TLR8, | |
| TLR9 | |
| TLR7, | Refractory diffuse large |
| TLR8, | B-cell lymphoma |
| TLR9 | |
| TLR7, | Diffuse large B-cell |
| TLR8, | lymphoma |
| TLR9 | |
| TLR7, | Macroglobulinemia |
| TLR8, | |
| TLR9 | |
| TLR7, | Muscular dystrophy |
| TLR8, | |
| TLR9 | |
| TLR7, | Waldenstrom's |
| TLR8, | macroglobulinemia is |
| TLR9 | refractory |
| TLR7, | Relapsed diffuse large |
| TLR8, | B-cell lymphoma |
| TLR9 | |
| TLR7, | Plaque psoriasis |
| TLR8, | |
| TLR9 | |
| TLR7, | Tumor |
| TLR9 | |
| TLR7, | Inflammatory bowel disease |
| TLR9 | |
| TLR7, | Systemic lupus |
| TLR9 | erythematosus |
| TLR7, | Rheumatoid arthritis |
| TLR9 | |
| TLR7, | Hyperlipidemia |
| TLR9 | |
| TLR7, | Multiple sclerosis |
| TLR9 | |
| TLR7, | Plaque psoriasis |
| TLR9 | |
| TLR9 | Autoimmune hepatitis |
| TLR9 | Metastatic head and neck |
| | squamous cell carcinoma |
| TLR9 | Metastatic breast cancer |
| GABA | Drug-resistant epilepsy |
| receptor | |
| GABA | epilepsy |
| receptor | |
| GAD | Parkinson's disease |
| GAD2, | Sciatica |
| GDNF, IL-10 | |
| GAD2, | Lumbar radiculopathy |
| GDNF, IL-10 | |
| GAD2, | Neuralgia |
| GDNF, IL-10 | |
| GAG | Mucopolysaccharidosis type |
| | II |
| GAG | HIV infection |
| GALC | Globoid cell leukodystrophy |
| galectin-3 | Osteosarcoma |
| GALGT2 | Duchenne muscular |
| | dystrophy |
| GALT | Galactosemia |
| GATA3 | asthma |
| GATA3 | Chronic obstructive |
| | pulmonary disease |
| GATA3 | Ulcerative colitis |
| GBA | Parkinson's disease |
| GBA | Gaucher disease |
| GBA | Fabry disease |
| GBA | Gaucher disease type II |
| GBA, | Gaucher disease |
| Phosphotransferases | |
| GC-C | Pancreatic cancer |
| GC-C | Colorectal cancer |
| GCDH | Glutaric acidemia |
| GCGR | Type 2 diabetes |
| GCGR | Type 2 diabetes |
| GD2 | Phyllodes |
| GD2 | Small cell lung cancer |
| TLR9 | Metastatic melanoma |
| TLR9 | Metastatic non-small cell |
| | lung cancer |
| TLR9 | Myasthenia gravis |
| TLR9 | Primary malignant liver |
| | tumor |
| TLR9 | Psoriasis |
| TLR9 | Pancreatic ductal |
| | adenocarcinoma |
| TLR9 | Pancreatic cancer |
| TLR9 | Mycosis fungoides |
| TLR9 | asthma |
| TLR9 | Small lymphocytic |
| | lymphoma |
| TLR9 | Advanced pancreatic ductal |
| | adenocarcinoma |
| TLR9 | Advanced non-small cell |
| | lung cancer |
| TLR9 | Advanced non-small cell |
| | lung cancer |
| TLR9 | Head and neck tumor |
| TLR9 | Head and Neck Squamous |
| | Cell Carcinoma |
| TLR9 | Eosinophilic asthma |
| TLR9 | Renal cell carcinoma |
| TLR9 | Ductal carcinoma |
| TLR9 | Breast cancer |
| TLR9 | Uveal melanoma |
| TLR9 | Skin melanoma |
| TLR9 | Cutaneous T-cell lymphoma |
| TLR9 | Refractory non-Hodgkin |
| | lymphoma |
| TLR9 | Refractory malignant solid |
| | tumor |
| TLR9 | Diffuse large B-cell |
| | lymphoma |
| TLR9 | Merkel cell carcinoma |
| TLR9 | Chronic obstructive |
| | pulmonary disease |
| GD2 | Ependymoma |
| GD2 | Neuroblastoma |
| GD2 | sarcoma |
| GD2 | Uveal melanoma |
| GD2 | Brain cancer |
| GD2 | Refractory neuroblastoma |
| GD2 | Diffuse intrinsic pontine |
| | glioma |
| GD2 | Glioma |
| GD2 | Rhabdomyosarcoma |
| GD2 | Melanoma |
| GD2 | Osteosarcoma |
| GD2 | High-grade glioma |
| GD2 | Recurrent Ewing sarcoma |
| GD2 | Recurrent neuroblastoma |
| GD2 | Recurrent osteosarcoma |
| GD2 | Glioblastoma multiforme |
| GD2 | Gaucher disease type II |
| GD2, IL15R | Small cell lung cancer |
| GD2, IL15R | Non-small cell lung cancer |
| GDNF | Parkinson's disease |
| GDNF | Amyotrophic lateral sclerosis |
| GFAP | Alexander disease |
| GFRA4 | Medullary thyroid cancer |
| GHR | Acromegaly |
| GHR | Acromegaly |
| GHRH | Anemia |
| GHRH | Cachexia |
| GITRL | Glioma |
| GJA1 | Arrhythmias |
| GJA1 | Myocardial disease |
| GJA1 | diabetic foot |
| TLR9 | Chronic lymphocytic |
| | leukemia |
| TLR9 | Chronic Hepatitis C |
| | Genotype 1 |
| TLR9 | Follicular lymphoma |
| TLR9 | Influenza virus infection |
| TLR9 | Squamous cell carcinoma |
| TLR9 | Ulcerative colitis |
| TLR9 | Macroglobulinemia |
| TLR9 | Locally advanced pancreatic |
| | adenocarcinoma |
| TLR9 | Locally advanced breast |
| | cancer |
| TLR9 | Colorectal cancer liver |
| | metastasis |
| TLR9 | Colorectal cancer |
| TLR9 | Conjunctival tumor |
| TLR9 | Hepatocellular carcinoma |
| TLR9 | Intrahepatic bile duct |
| | carcinoma |
| TLR9 | Relapsed mantle cell |
| | lymphoma |
| TLR9 | Relapsed marginal zone |
| | lymphoma |
| TLR9 | Relapsed grade 3a follicular |
| | lymphoma |
| TLR9 | Non-small cell lung cancer |
| TLR9 | Nonalcoholic steatohepatitis |
| TLR9 | Indolent B-cell |
| | non-Hodgkin lymphoma |
| TLR9 | Multiple sclerosis |
| TLR9 | Hepatitis C |
| TLR9 | T-cell lymphoma |
| TLR9 | EGFR-positive non-small |
| | cell lung cancer |
| TLR9 | CD20-positive non-Hodgkin |
| | lymphoma |
| TM4SF1 | Digestive system tumor |
| TMC1 | Hearing loss |
| TMPRSS6 | Polycythemia vera |
| TMPRSS6 | High-risk myelodysplastic |
| | syndrome |
| TMPRSS6 | Thalassemia |
| TNF | psoriasis |
| GJA1 | Corneal damage |
| GJB2 | Hearing loss |
| GLA | Fabry disease |
| GLA | Glycogen storage disease |
| | type II |
| GLA | Chagas cardiomyopathy |
| GlcCer | Neurodegenerative disease |
| GlcCer | Gaucher disease |
| GlcCer | Gaucher disease type I |
| GLI2 | Tumor |
| GLI2 | Prostate cancer |
| GLIPR1 | Prostate cancer |
| Globo H | Gastric cancer |
| GLP-1R | Weight loss |
| GLP-1R | Obesity |
| GLP-1R | Type 2 diabetes |
| glucokinase | Type 2 diabetes |
| Glucokinase, | Diabetes |
| insulin | |
| GluK2, | Temporal lobe epilepsy |
| Kainic acid | |
| receptor | |
| GluK2, | Epilepsy |
| Kainic acid | |
| receptor | |
| Glycogen | Glycogen storage disease |
| | type II |
| GM2 | Solid tumor |
| (ganglioside | |
| M2) | |
| GM-CSF | Bladder cancer |
| GM-CSF, | Colorectal cancer |
| decorin | |
| GM-CSF, | Advanced solid tumor |
| IFNα2, IL-12, | |
| IL-15 | |
| GM-CSF, | Solid tumor |
| IFNα2, IL-12, | |
| IL-15 | |
| GM-CSF, | Melanoma |
| IFNα2, IL-12, | |
| IL-15 | |
| GM-CSF, | Tumor |
| RYBP | |
| TNF | Renal cell carcinoma |
| TNF | Prostate cancer |
| TNF | Brain cancer |
| TNF | Huntington's disease |
| TNF | Melanoma |
| TNF | Alzheimer's disease |
| TNF-α | Tumor |
| TNF-α | Rectal cancer |
| TNF-α | Psoriasis |
| TNF-α | Pancreatic cancer |
| TNF-α | Inflammation |
| TNF-α | Periodontal disease |
| TNF-α | Retinitis pigmentosa |
| TNF-α | Esophageal cancer |
| TNF-α | Sarcoma |
| TNF-α | Prostate cancer |
| TNF-α | Rheumatoid arthritis |
| TNF-α | Crohn's disease |
| TNF-α | Locally advanced prostate |
| | cancer |
| TNF-α | Colorectal cancer |
| TNF-α | Melanoma |
| TNF-α | Melanoma |
| TNF-α | Osteosarcoma |
| TNSALP | Hypoalkaline phosphatase |
| TOP1 | Tumor |
| TP63 | Colorectal cancer |
| TPP1 | Neuronal ceroid |
| | lipofuscinosis |
| GNE | Distal myopathy |
| GNE | Hereditary inclusion body |
| | myopathy |
| GOPC | Tumor |
| GOX | Primary hyperoxaluria type 1 |
| GOX | Primary hyperoxaluria |
| GOX | Kidney Stone Disease |
| gp100 | Melanoma |
| GPC1 | Pancreatic cancer |
| GPC2 | Small cell lung cancer |
| GPC2 | Neuroblastoma |
| GPC2 | Refractory neuroblastoma |
| GPC2 | High-risk neuroblastoma |
| GPC2 | Recurrent neuroblastoma |
| GPC2 | Bladder cancer |
| GPC3 | Ovarian cancer |
| GPC3 | Squamous cell lung cancer |
| GPC3 | Hepatocellular carcinoma |
| GPC3 | Lung cancer |
| GPC3 | Cholangiocarcinoma |
| GPC3, IL-15 | Solid tumor |
| GPC3, IL-15 | Hepatocellular carcinoma |
| GPC3, IL15R | Liver cancer |
| GPC3, | Solid tumor |
| IL-2Rβ | |
| GPC3, | Hepatocellular carcinoma |
| NKp46 | |
| GPCR | Retinitis pigmentosa |
| GPCR, | Novel Coronavirus Infection |
| β-adrenoceptors | |
| GPCR, | Heart failure |
| β-adrenoceptors | |
| GPCR, | Chronic fatigue syndrome |
| β-adrenoceptors | |
| GPCR, | Dilated cardiomyopathy |
| β-adrenoceptors | |
| TPP1 | Neuronal ceroid |
| | lipofuscinosis 2 |
| TRAIL | Metastatic non-squamous |
| | non-small cell lung cancer |
| TRAIL | Pancreatic cancer |
| TRAIL | Hepatocellular carcinoma |
| TRAIL | Lung adenocarcinoma |
| TRBC1 | Immunoblastic |
| | lymphadenopathy |
| TRBC1 | Relapsed T-cell lymphoma |
| TRBC1 | TRBC1-positive T-cell |
| | lymphoma |
| TRBC2 | Peripheral T-cell lymphoma |
| TRKA | pain |
| Trop-2 | Breast cancer |
| Trop-2 | Colon cancer |
| Trop-2 | Lung cancer |
| Trop-2 | Malignant epithelial tumor |
| TRPV1 | Eye pain |
| TRPV1 | Dry eye syndrome |
| TRPV1 | Complex Regional Pain |
| | Syndrome |
| Trypsin | Cystic fibrosis |
| Trypsin | Chronic obstructive |
| | pulmonary disease |
| Trypsin | Respiratory syncytial virus |
| | infection |
| Trypsin | Emphysema |
| Trypsin | Alpha-1 antitrypsin |
| | deficiency |
| TSHR | Thyroid cancer |
| TSLPR | B-cell leukemia |
| TTK | Corneal disease |
| TTR | Transthyretin Amyloidosis |
| TTR | Transthyretin cardiac |
| | amyloidosis |
| TTR | Transthyretin-associated |
| | familial amyloid |
| | polyneuropathy |
| TTR | Hematopoietic stem cell |
| | transplantation |
| GPRC5D | Refractory plasma cell |
| | myeloma |
| GPRC5D | Plasma cell leukemia |
| GPRC5D | Plasma cell leukemia |
| GPRC5D | Relapsed Multiple myeloma |
| GPRC5D | Multiple myeloma |
| GPVI | Vascular disease |
| GR | Cushing syndrome |
| GR | Metabolic disease |
| GR | Type 2 diabetes |
| GRB2 | Endometrial cancer |
| GRB2 | Fallopian tube cancer |
| GRB2 | Ovarian epithelial carcinoma |
| GRB2 | Peritoneal cancer |
| GRB2 | Recurrent solid tumor |
| GSTP1 | Pancreatic cancer |
| GSTP1 | Colorectal cancer |
| GSTP1 | Non-small cell lung cancer |
| GSTP1 | KRAS-mutated non-small |
| | cell lung cancer |
| GTPCH1 | Immune system disorders |
| GUCY2D | Leber congenital amaurosis |
| | type 1 |
| GUCY2D | Leber congenital amaurosis |
| GYS1 | Glycogen storage disease |
| | type III |
| GYS1 | Lafora disease |
| GYS1 | Glycogen storage disease |
| | type II |
| GYS1 | Glycogen storage disease |
| | type II |
| H1 receptor, | Upper respiratory tract |
| Opioid | allergies |
| receptors | |
| H1 receptor, | Common cold |
| Opioid | |
| receptors | |
| H1 receptor, | Cough |
| Opioid | |
| receptors | |
| HAAH | Hematologic ma5lignancy |
| TTR | Stargardt disease |
| TTR | Senile cardiac amyloidosis |
| TTR | Transthyretin amyloid |
| | cardiomyopathy |
| TTR | Familial amyloid |
| | neuropathy |
| TTR | Polyneuropathy |
| TTR | Amyloidosis |
| TUBB4A | Type 6 myelin-reduced |
| | leukodystrophy |
| TUSC2 | Breast cancer |
| TUSC2 | Lung cancer |
| TUSC2 | Non-small cell lung cancer |
| TYK2 | Autoimmune disease |
| TYK2 | Novel Coronavirus Infection |
| TYMP | Mitochondrial |
| | encephalomyopathy |
| TYMS | Pancreatic cancer |
| TYMS | Gastric cancer |
| TYMS | Prostate cancer |
| TYMS | Ovarian cancer |
| TYMS | Glioblastoma |
| TYMS | Bone cancer |
| TYR | Hyperpigmentation |
| TYR | Melanoma |
| UBE3A | Angelman syndrome |
| UCN2 | Heart failure |
| UCN2 | Abdominal obesity and |
| | metabolic syndrome |
| UCN2 | Obesity |
| UCN2 | Nonalcoholic steatohepatitis |
| UCN2 | Type 2 diabetes |
| UGT1A1 | Nonalcoholic steatohepatitis |
| UGT1A1 | Crigler-Najjar syndrome |
| HAAH | Solid tumor |
| HAAH | Glioblastoma |
| harmonin | Usher syndrome |
| HBA1 | Beta thalassemia |
| HBG1 | Sickle cell disease |
| HBG1 | Beta thalassemia |
| HBG1, | Sickle cell disease |
| HBG2 | |
| HBG1, | Beta thalassemia |
| HBG2 | |
| HBG2 | Sickle cell disease |
| HBG2 | Beta thalassemia |
| HBsAg | Hepatitis B virus-related |
| | hepatocellular carcinoma |
| HBsAg | Hepatitis B |
| HBsAg | diabetic foot |
| HBsAg | Hepatocellular carcinoma |
| HBsAg | Hepatitis D |
| HBV pol | Hepatitis B |
| HBV RNA | Hepatitis B |
| HBV-X | Hepatitis B |
| HBV-X | Chronic hepatitis B |
| HBV-X | Hepatitis D |
| HBX | Hepatitis B |
| HCCS1 | Hepatocellular carcinoma |
| HDAC6 | Tumor |
| HDGFL3 | Tumor |
| hemagglutinin | Niemann-Pick disease type |
| | C2 |
| Hemoglobin | Alpha thalassemia |
| HbA | |
| Hemoglobins | Transfusion-dependent |
| | beta-thalassemia |
| Hemoglobins | Sickle cell disease |
| Hemoglobins | Thalassemia |
| Hemoglobins, | Sickle cell disease |
| KLKB1, TTR | |
| Hemoglobins, | Beta thalassemia |
| KLKB1, TTR | |
| ULBP1 | Hematologic malignancy |
| ULBP1 | Advanced solid tumor |
| ULBP1 | Advanced cancer |
| ULBP1 | Refractory cancer |
| UNC13A | Amyotrophic lateral |
| | sclerosis |
| UNC13A | Frontotemporal dementia |
| UPF1 | Amyotrophic lateral |
| | sclerosis |
| USH2A | Usher syndrome |
| UTRN | Duchenne muscular |
| | dystrophy |
| VAMP1 + | Nasopharyngeal tumor |
| VAMP2 | |
| VCAM1 | Inflammatory bowel disease |
| VDR | Multiple sclerosis |
| VEGF | Diabetic retinopathy |
| VEGF | Wet age-related macular |
| | degeneration |
| VEGF | Macular degeneration |
| VEGF-A | Myocardial ischemia |
| VEGF-A | Peripheral arterial disease |
| VEGF-A | Diabetic macular edema |
| VEGF-A | Retinal vein occlusion |
| VEGF-A | Wet age-related macular |
| | degeneration |
| VEGF-A | Breast cancer |
| VEGF-A | Colorectal cancer |
| VEGF-A | Macular degeneration |
| VEGF-A | Liver cancer |
| VEGF-A | Malignant ascites |
| VEGF-A, | Angina |
| VEGFR | |
| VEGF-A, | Ischemia |
| VEGFR | |
| VEGF-A, | Coronary heart disease |
| VEGFR | |
| VEGF-C | Lymphedema |
| VEGF-D | Peripheral vascular disease |
| VEGFR | Tumor |
| Hepc | Tumor |
| Hepc | End-stage renal disease |
| Hepc | Inflammation |
| Hepc | Anemia |
| Hepc | endotoxemia |
| Hepc | Chronic |
| Hepc | Anemia with chronic kidney |
| | disease |
| Hepc, IL-10, | Kidney disease |
| TGF-β | |
| HER2 | Endometrial cancer |
| HER2 | Endometrial cancer |
| HER2 | Tumor metastasis |
| HER2 | Tumor |
| HER2 | Tumor |
| HER2 | Pancreatic cancer |
| HER2 | Inflammatory breast tumor |
| HER2 | Small cell lung cancer |
| HER2 | Gastroesophageal junction |
| | cancer |
| HER2 | Gastric cancer |
| HER2 | Head and neck tumor |
| HER2 | Breast cancer |
| HER2 | Prostate cancer |
| HER2 | Urothelial carcinoma |
| HER2 | Brain metastases |
| HER2 | Ovarian epithelial carcinoma |
| HER2 | Ovarian cancer |
| HER2 | Squamous cell carcinoma |
| HER2 | Colorectal cancer |
| HER2 | Glioma |
| HER2 | Mesothelioma |
| VEGFR | Head and Neck Squamous |
| | Cell Carcinoma |
| VEGFR | Retinal disease |
| VEGFR | Macular degeneration |
| VEGFR | Nasopharyngeal carcinoma |
| VEGFR1 | Preeclampsia |
| VEGFR1 | Age-related macular |
| | degeneration |
| VEGFR1 | Choroidal |
| | neovascularization |
| VEGFR1 | Macular degeneration |
| VEGFR1, | Mesothelioma |
| VEGFR2, | |
| VEGFR3 | |
| VEGFR2 | Tumor |
| VEGFR2 | Wet age-related macular |
| | degeneration |
| VEGFR2 | Hepatocellular carcinoma |
| Viral | Hepatitis B |
| proteins | |
| Viral | Chronic hepatitis B |
| proteins | |
| Viral | Marburg virus disease |
| proteins | |
| VP24 | Marburg virus disease |
| VP24, Viral | Ebola virus disease |
| RNA | |
| VP35, Viral | Ebola virus disease |
| RNA | |
| VSTM1 | Tumor |
| vWF | Hemophilia A |
| vWF | Thrombotic |
| | thrombocytopenia purpura |
| vWF | Thrombotic |
| | microangiopathy |
| vWF | Thrombosis |
| vWF | Hemolytic uremic syndrome |
| vWF | Intracranial embolism |
| vWF | Carotid artery stenosis |
| vWF | Acute ischemic stroke |
| vWF | Acquired thrombotic |
| | thrombocytopenia purpura |
| vWF | Coronary artery thrombosis |
| HER2 | Chemotherapy-induced |
| | nausea and vomiting |
| HER2 | Osteosarcoma |
| HER2 | Cervical cancer |
| HER2 | Hepatocellular carcinoma |
| HER2 | Lung cancer |
| HER2 | Obesity |
| HER2 | Non-small cell lung cancer |
| HER2 | Ductal carcinoma |
| HER2 | Bile duct tumor |
| HER2 | Bladder cancer |
| HER2 | HER2-positive gastric cancer |
| HER2 | HER2-positive breast cancer |
| HER2 | Type 2 diabetes |
| HER2, HER3 | Triple-negative breast cancer |
| HER2, HER3 | Meningeal tumor |
| HER2, HER3 | HER2-positive breast cancer |
| HER2, HER3 | HER2-negative breast cancer |
| HER2, | Ovarian cancer |
| IL-13Rα2 | |
| HER2, IL15R | Gastric cancer |
| HER2, IL15R | Breast cancer |
| HER2, | Solid tumor |
| NKG2D | |
| HER2, PD-1 | Solid tumor |
| HER3 | Tumor |
| HER3 | Breast cancer |
| HER3 | Colon cancer |
| HER3 | Lung cancer |
| hERG | Atrial fibrillation |
| HEXA, | Tay-Sachs disease |
| HEXB | |
| HEXA, | Sandhoff disease |
| HEXB | |
| HEXA, | GM2 gangliosidosis |
| HEXB | |
| HGF | Autoimmune disease |
| HGF | Tumor |
| HGF | Myocardial ischemia |
| HGF | Lower limb ischemia |
| vWF | Von Willebrand disease |
| vWF | Von Willebrand disease |
| | type 2 |
| WASP | Angina |
| WASP | Wiskott-Aldrich syndrome |
| WT1 | Acute myeloid leukemia |
| WT1 | Myelodysplastic syndrome |
| WT1 | Multiple myeloma |
| XIAP | Pancreatic cancer |
| XIAP | Breast cancer |
| XIAP | Chronic lymphocytic |
| | leukemia |
| XIAP | Acute myeloid leukemia |
| XIAP | Acute myelomonocytic |
| | leukemia |
| XIAP | Hepatocellular carcinoma |
| XIAP | Non-small cell lung cancer |
| XIAP | Malignant epithelial tumor |
| XIAP | Multiple sclerosis |
| XIAP | B-cell lymphoma |
| XKR8 | Pancreatic cancer |
| XKR8 | Colon cancer |
| XLRS1 | Retinoschisis |
| XO | Gout |
| YAP1 | Advanced solid tumor |
| ZSCAN4 | Dyskeratosis congenita |
| ZSCAN4 | Pancytopenia |
| ZSCAN4 | Bone marrow failure disease |
| α-glucosidase | Glycogen storage disease |
| | type II |
| α-Klotho | Amyotrophic lateral |
| | sclerosis |
| α-synuclein | Parkinson's disease |
| α-synuclein | Parkinson's disease |
| α-synuclein | Multiple System Atrophy |
| αvβ3 | Breast cancer |
| αvβ3 | Melanoma |
| αvβ6 | Tumor |
| ανβ6 | Pancreatic cancer |
| HGF | Peripheral arterial disease |
| HGF | Diabetic peripheral |
| | neuropathy |
| HGF | Diabetic vascular disease |
| HGF | Diabetic neuropathy |
| HGF | Diabetic nephropathy |
| HGF | Breast cancer |
| HGF | Ischemia |
| HGF | Chronic limb-threatening |
| | ischemia |
| HGF | Ulcer |
| HGF | Amyotrophic lateral sclerosis |
| HGF | Coronary heart disease |
| HGF | Type 1 diabetes |
| HIF-1α | Peripheral arterial disease |
| HIF-1α | Lymphoma |
| HIF-1α | Hepatocellular carcinoma |
| HIF-1α | Liver cancer |
| HIF-1α | Liver cancer |
| HIF-1α | Malignant epithelial tumor |
| ανβ6 | Amyotrophic lateral |
| | sclerosis |
| β2-adrenergic | Glaucoma |
| receptor | |
| β2-adrenergic | Ocular hypertension |
| receptor | |
| β-galactosidase | Gangliosidosis |
| β-galactosidase | GM1 gangliosidosis |
| β-globin | Sickle cell anemia |
| β-globin | Beta thalassemia |
| Amiloride- | Cystic fibrosis |
| sensitive | |
| sodium | |
| channel | |
| Amiloride- | Chronic bronchitis |
| sensitive | |
| sodium | |
| channel | |
| Intercellular | Inflammation |
| adhesion | |
| molecule | |
| Viral RNA | Pseudohyperkalemia Cardiff |
| | type |
| Viral RNA | Hepatitis C |
| Viral | Merkel cell carcinoma |
| Protein | |
| Viral | Resectable Merkel cell |
| Protein | carcinoma |
| Collagen I | Achilles tendinitis |
| Immunoglobulin | Lymphoma |
| light | |
| chain kappa | |
| Immunoglobulin | Multiple myeloma |
| light | |
| chain kappa | |
| Immunoglobulin | leukemia |
| light | |
| chain kappa | |
| HIF-1α, | Tumor |
| transcription | |
| factors, and | |
| related | |
| regulatory | |
| factors | |
| HIF-2α | Renal cell carcinoma |
| HIF-2α | Neuroblastoma |
| Histone H3 | Diffuse midline glioma, H3 |
| | K27M-mutant |
| HIV envelope | AIDS |
| protein gp120 | |
| HIV gag | AIDS |
| HIV gag | HIV infection |
| HIV-1 | AIDS |
| integrase | |
| HIV-1 | HIV infection |
| integrase | |
| HIV-1 pol | Opportunistic infections |
| HIV-1 pol | AIDS |
| HIV-1 pol | Hepatitis C |
| HIV-1 tat | HIV infection |
| HIV RNase | HIV infection |
| H | |
| HJV, Hepc | anemia |
| Endotoxin | Prostate cancer |
| A | |
| Coagulation | Hemophilia A |
| Factor VIII | |
| Human | Solid tumor |
| endogenous | |
| retroviral | |
| elements | |
| Growth | Neurodegenerative disease |
| factor | |
| receptors | |
| Cell | Inflammation |
| adhesion | |
| molecules | |
| Cell | Septic shock |
| adhesion | |
| molecules | |
| SARS-CoV-2 | Novel Coronavirus Infection |
| Antigen | |
| Folate | Tumor |
| receptor | |
| Apolipoprotein | Alzheimer's disease |
| Tumor | Liver cancer |
| antigen | |
| MG7 | |
| Transforming | Tumor |
| growth | |
| factor β | |
| receptor | |
| Transcription | Larsen syndrome |
| factors | |
| and related | |
| regulatory | |
| factors | |
| Transcription | Fuchs endothelial corneal |
| factors | dystrophy type 3 |
| and related | |
| regulatory | |
| factors | |
| Transcription | Tumor |
| factors | |
| and related | |
| regulatory | |
| factors, | |
| c-Jun | |

### EXAMPLES

The present disclosure is further described below by way of examples, but the present disclosure is not limited to the scope of the examples. The experimental methods without specific conditions in the following examples are performed according to conventional manners and conditions, or according to product specifications.

### Example 1. Screening C12-279 Protein

As shown in FIGS. 1A and 1B, the Cas12i protein C12-279 was finally obtained through screening, analysis, and designing using a complex bioinformatics manner.

An amino acid sequence of the C12-279 protein (SEQ ID NO: 696) is:

(SEQ ID NO: 696)
IFVVLSVNQFRCNPNAFRQASMKTDDEKKDTAIKSYTSWLLPNDRKEKDMVRSFLALD
QGSRFFFDLMQAWYGGLTPDIIKSVTKERDLVDLWCAIYWFRPMKKELASHPIDRVDLA
KTFQRYYGGPASDVVQEYLSASIGEDCCWNDCRQKYQEFCKNLGVDFTPDLKTLVREK
LIAVALNDGSQAAISNLFGNGGKEDRNVKVNICKKILIALEKAGKSVKYVRDVQSIILKC
ANAKDREDFNRIYADKNGRPGTLLLLLDRKGENTYDAEVLKRYLRKTIKSKNAPLVWN
YNQKLFEYIERGIRNLLRRTINYDAWAWGEMWRVGHTPLQTKATRNYHFAQEQLKRHS
DIAAASQSSHAIAVALNEFFESPFFDGENPFTICPHHLGKNLEKLFKAWADEKNQTDEED
AGETAITDYCAEFKDGFDREPIRNLLRYVYASLRSKYSAKELIQAAKYNQQFERYQRQK
AHPVTPDNQGYTWPEAVIKPSKAERKNRENSLDTRIWVSVKLLQKDGTWEKHHIPFYN
LRFFEEVYAAASIAEDMPSPTFRNSRFGFKLPKLLNTHAKINKKKKDGKKDKKAVNAAK
REARIYLAAKEGRLPAPSVPLDKLSAVIARVNGKFQVTVPVKFKVNKQPKYPLPQLGQII
LGYDQNLTANHAWALFEVVTEDTPGAHFCRNQYLRVLKTGQVRSITKTKDGQEIDQLSY
DGLEYKEYGEWRKQAKKFASQWYITTKVRKGKETTLLSQPALERFESIEKYKPALYRFN
KEYARLLLAVIRRKTLNELEEIRPEIFRLVEQGFGICRFGSLSLSSFEAIRAAKAVIYGYFST
ALKHDHNTPITDDERQAFDPELFGLLNKLETLRQNKGEEKINRAANALIRIALENKADFI
RGEGDLPTTNKSVKKGQNSRSMDWLARGLFNKICQLAVMHNIIPTAANPHSTSHQDPFE
HNKDFDDPQPAMKCRYAEFKIEDIGDSVLERLGTSLRNAKAGQTGAYYYKGAQDFLAH
YGLQDIEEELKKCRRGRKANMPCWELQKRLIQKLGSKDAVVYIPMRGGRIYLATHSVTT
GAKPIIFNGEEVWVSNADEIAAVNIGLTIIPTSKKDEEHPDRENDDKAIRKSVRRPRKSGE
RRSGDKMPATARKT.

A predicted structure of the C12-279 protein is shown in FIG. 24.

### Example 2. Preparation and Purification of C12-279 Protein

### 1. Vector Construction

A pET28a vector plasmid was double digested with BamHI and XhoI, and a linearized vector was recovered by agarose gel electrophoresis. Using the prepared pXC12-279-GFPPAM-DR5 plasmid (SEQ ID NO: 697) as a template, DNA fragments containing coding sequences of the C12-279 protein was obtained by PCR amplification with primers ChkCas-pET28-PF1 (SEQ ID NO: 699) and ChkCas-pET28-PR1 (SEQ ID NO: 700). The DNA fragments were inserted into a cloning region of the pET28a vector by homologous recombination (New England Biolabs (NEB™), Gibson Assembly® Master Mix) to construct a recombinant vector C12-279-pET28a-01 (SEQ ID NO: 698). A reaction solution was transformed into Stbl3 competent cells, coated on an LB plate containing kanamycin sulfate, and cultured at 37° C. overnight, and clones were picked for sequencing and identification.

Positive clones with a correct sequence were selected and cultured overnight. Plasmids were extracted and transformed into an expression strain Rosetta (DE3), and then coated on the LB plate containing kanamycin sulfate and cultured at 37° C. overnight.

### 2. Protein Expression

A single clone was selected and inoculated into 5 mL of LB culture medium containing kanamycin sulfate and cultured at 37° C. overnight.

The selected single clone was reinoculated into 500 mL of LB culture medium containing kanamycin sulfate at a ratio of 1:100, cultured at 220 rpm and 37° C. until OD reached 0.6, added with IPTG to a final concentration of 0.2 mM, and induced at 16° C. for 24 h.

After rinsing with 15 mL PBS, the bacteria were collected by centrifugation, added with a lysis buffer for sonication disruption, a supernatant containing the recombinant protein was obtained by centrifuging at 10,000 g for 30 min, and the supernatant was filtered through a 0.45 μm filter membrane before being applied to column purification.

### 3. Protein Purification

The expressed recombinant protein has 1240 amino acids (aa) and a structure of His tag-NLS-C12-279-SV40 NLS-nucleoplasmin NLS. Using 6×His tag at an N-terminal as purification tags, the C12-279 recombinant protein was obtained by Immobilized Metal Ion Affinity Chromatography (IMAC) (Ni Sepharose™ 6 Fast Flow affinity chromatography media (Cytiva™)) and heparin affinity chromatography (POROS™ Heparin 50 μm chromatographic column, Thermo Scientific™). The purified recombinant protein was detected by SDS-PAGE electrophoresis, and results are shown in FIG. 2.

### Example 3. In Vitro Cleavage of PAM Library and Grabbing of PAM by C12-279 Protein

In this example, sgRNA containing a specific guide sequence and the C12-279 recombinant protein prepared and purified as described in Example 2 were mixed to cleave an in vitro cleavage substrate (containing a spacer sequence and a 7nt random sequence) (as shown in FIG. 3). After incubation at 37° C., purification and library construction were performed, followed by NGS sequencing to determine the PAM sequence of C12-279. Specific operations were as follows.

### In Vitro Cleavage Substrate

The designed in vitro cleavage substrate sequence is as follows:

(SEQ ID NO: 701)
ggagttcagacgtgtgctcttccgatctcagcacaaaaggaaactcaccc
taactgtaaagtaattgtgtgttttgagactataaatatgcatgcgagaa
aagccttgtttgccaccatGGAACGGCTCGGAGATCATCATTGCGNNNNN
NNgtgagcaagggcgaggagctgttcaccggggtggtgcccatcctggtc
gagctggacggcgacgtaaacggccacaagttcagcgtgtccggcagatc
ggaagagcacacgtctgaactcc.

In the sequence, N represents any one of A, T, C, and G.

A double-stranded DNA containing the above sequence was prepared by PCR amplification and used as the in vitro cleavage substrate.

The cleavage substrate was sent to a sequencing company for PCR-free library construction and NGS sequencing. A complexity and abundance analysis on the PAM library composed of the 7nt random sequence were analyzed. The result is as follows.

Compositions of the four bases A, T, G, and C are basically the same. At the same time, the PAM library composed of the 7nt random sequence contains 4\hat{ }7=16384 different combinations, all of which (100%) are detected. The complexity and abundance of the PAM library are qualified.

B. Preparation of sgRNA

The sgRNA (C12-279-sgRNA) containing a specific guide sequence was synthesized by in vitro transcription at 37° C. in a system containing T7 RNA transcriptase, four ribonucleotide triphosphates, and a DNA template with a T7 promoter. A transcription product was precipitated and purified with LiCl. The sgRNA sequence is as follows:

>C12-279-sgRNA
(SEQ ID NO: 702)
5′-gtaatgcgtctcccattgacgcGTGAGCAAGGGCGAGGAGCTGTT
C-3′.
>C12-279-sgRNA-Rev
(SEQ ID NO: 703)
5′-gtaatgcgtctcccattgacgcCGCAATGATGATCTCCGAGCCGT
TCC-3′.
The sgRNA scaffold sequence (DR sequence) is:
(SEQ ID NO: 704)
5′-gtaatgcgtctcccattgacgc-3′.

The uppercase bases are the guide sequence of the sgRNA.

### C. NGS Library Construction and PAM Analysis

### 1) PAM Library Cleavage and T4 DNA Polymerase Treatment

Reaction systems containing the C12-279 protein, two different sgRNAs, the in vitro cleavage substrate, and a buffer were prepared (as shown in Table 1) and reacted at 37° C. for 3 h and at 75° C. for 15 min).

TABLE 1
Reaction system for in vitro cleavage reaction

| | Addition |
|---|---|
| Components | amount (μL) |
| 10× Cut Buffer (500 mM Tris-HCl PH 8.0, | 5 |
| 2M NaCl, 100 mM MgCl₂, 10 mM DTT) | |
| Cleavage substrate (59.5 ng/μL) | 36.5 |
| C12-279- sgRNA or C12-279-sgRNA-Rev | 2.8 μg |
| C12-279 protein (10 mg/mL) | 0.5 |

### 2) T4 DNA Polymerase Treatment for Blunting Cleavage Product

The T4 DNA Polymerase (Thermo Scientific™) was added to the cleavage product. The specific reaction system was shown in Table 2. After addition, the reaction was performed at 37° C. for 20 min and at 85° C. for 10 min.

TABLE 2
Reaction system for blunting the C12-279 cleavage product

| | Addition |
|---|---|
| Component | amount (μL) |
| C12-279 cleavage product | 50 |
| 5× T4 DNA Polymerase Buffer | 13 |
| T4 DNA Polymerase | 1 |
| dNTP(10 mM) | 0.65 |
| ddH₂O | 0.35 |

### 3) 3′ End A-Tailing and Addition of Biotin-Labeled Adapter

a. 78 μL SPRISelect™ Beads (Beckman COULTER™) were added to the T4 DNA Polymerase reaction product and mixed well, the mixture was placed at room temperature for 5 min, the product was moved to a magnetic rack for adsorption for 5 min, the supernatant was transferred to a new 1.5 mL tube, 39 μL SPRISelect™ Beads (Beckman COULTER™) was added and mixed well, and placed at room temperature for 5 min. The product was moved to the magnetic rack for adsorption for 5 min. A supernatant was discarded, then the product was washed twice with 85% ethanol, placed at room temperature for 10 min for air drying, and added with 50 μL ddH₂O for elution.

b. Using the SynplSeq DNA Library Prep Kit for Illumina® library prep kit, 3′ A-tailing was performed on the product in step a according to the system in Table 3 at 37° C. for 10 min, 65° C. for 20 min, and 4° C. for later use.

TABLE 3
3′ A-tailing of C12-279 cleavage product

| | Addition |
|---|---|
| Component | amount (μL) |
| Cleavage products of C12-279 | 49 |
| with different sgRNA | |
| Enhancer | 1.5 |
| End Prep Mix | 6 |
| End Prep Buffer 1 | 3 |
| ddH₂O | 0.5 |
| Total | 60 |

c. Adapter 1 (obtained by annealing an upstream primer: 5′Biosg/gttgacatgctggattgagacttcctacactctttccctacgacgctcttccgatc*t (SEQ ID NO: 705) and a downstream primer: gatcggaagagcgtcgtgtagggaaaga gtgtaggaagtctcaatccagcatgtcaac (SEQ ID NO: 706)) was added according to the system in Table 4 and reacted at 20° C. for 30 min and at 16° C. overnight. The reaction product was purified using SPRISelect™ Beads.

Biosg is a biotin modification, and “*” represents thiolation.

TABLE 4
Reaction system added with Adapter 1

| Component | Addition amount (μL) |
|---|---|
| Reaction product in step b | 60 |
| Adapter 1 | 2 |
| DNA Ligase | 1.2 |
| 3x Ligation Buffer 1 | 33 |
| ddH₂O | 3.8 |
| Total | 100 |

d. The reaction product was purified using streptavidin-labeled magnetic beads Dynabeads® M-280 Streptavidin (Invitrogen™, Thermo Fisher Scientific).

e. Recover PCR

The primers in Table 5 were designed, and the Recover PCR reaction was performed using Q5® Hot Start High-Fidelity 2× Master Mix (NEB™) according to the system in Table 6 and the reaction program in Table 7.

TABLE 5
Recover PCR primers

| Primer ID | sequence |
|---|---|
| Recovery PCR Forward | ggagttcagacgtgtgctc (SEQ ID NO: 707) |
| Recovery PCR Reverse | gttgacatgctggattgagacttc (SEQ ID NO: 708) |

TABLE 6
Recover PCR reaction system

| Component | Addition amount (μL) |
|---|---|
| Streptavidin-labeled magnetic beads | 22.5 |
| purification product | |
| Recovery PCR Forward (10 uM) | 2.5 |
| Recovery PCR Reverse (10 uM) | 2.5 |
| Q5 ® Hot-Start 2x Master Mix | 22.5 |
| ddH₂O | Up to 50 |

TABLE 7
Recover PCR reaction program

| Reaction temperature | Duration | | Number of cycles |
|---|---|---|---|
| 98° C. | 2 | min | 1 |
| 98° C. | 10 | sec | 12 |
| 61° C. | 30 | sec | |
| 72° C. | 2 | min | |
| 72° C. | 2 | min | 1 |

| | |
|---|---|
| 4° C. | ∞ |

f. The Recover PCR product was moved to the magnetic rack and adsorbed for 5 min, the supernatant was moved to a new 1.5 mL centrifuge tube, 3 μL of the Recovery PCR product was taken, and 148.5 μL ddH₂O was added to dilute.

g. Index PCR

The primers in Table 8 were selected, and the Index PCR was performed according to the system in Table 9 and the reaction program in Table 10.

TABLE 8
Index PCR primers

| Primer | Sequence |
|---|---|
| IF501 | aatgatacggcgaccaccgagatctacactatagcctacactctttccctacacgacg |
| | (SEQ ID NO: 709) |
| IR701 | caagcagaagacggcatacgagatcgagtaatgtgactggagttcagacgtgtgctc |
| | (SEQ ID NO: 710) |

TABLE 9
Index PCR reaction system

| Component | Addition amount (μL) |
|---|---|
| Recovery PCR dilution product | 12 |
| IF501(10 uM) | 4 |
| IR701(10 uM) | 4 |
| Q5 ® Hot-Start 2x Master Mix | 20 |
| Total | 40 |

TABLE 10
Index PCR reaction program

| | | |
|---|---|---|
| Reaction temperature | Duration | Count of cycles |

| | | | |
|---|---|---|---|
| 98° C. | 2 | min | 1 |
| 98° C. | 10 | s | 12 |
| 60° C. | 30 | s | |
| 72° C. | 2 | min | |
| 72° C. | 2 | min | 1 |

| | |
|---|---|
| 4° C. | ∞ |

h. 0.7×SPRISelect™ Beads were added to the Index PCR product for product purification, 38 μL ddH₂O was added for elution, the concentration was measured using a Qubit® fluorometer (Thermo Fisher Scientific), and then sent for NGS sequencing.

i. Analysis of NGS results: through the NGS sequencing and analysis with WebLogo software by referring to the document (A compact Cas9 ortholog from Staphylococcus auricularis (SauriCas9) expands the DNA targeting scope. PLoS biology, 2020, 18(3), e3000686), the identified motifs were obtained as shown in FIGS. 4 and 5. Both FIGS. 4 and 5 demonstrate that the PAM recognized by the C12-279 is 5′-TTN-3′.

### Example 4. Verification of PAM Based on In Vivo Editing Activity of C12-279 Protein in Bacteria

In this example, a plasmid library containing a 7nt random sequence was first constructed, and then a bacterial expression plasmid containing the C12-279 protein coding sequence was constructed. After the expression plasmid was transformed into bacteria to prepare a competent cell, the 7nt random sequence plasmid library was electroporated. If the plasmid in the 7nt random sequence library could be recognized and targeted by C12-279, the plasmid may be removed from the library and the corresponding bacteria could not grow, as shown in FIG. 6.

The specific operations were as follows.

### 1. Construction of 7nt Random Sequence Plasmid Library

pLVX-EF1a-BSD vector plasmid (SEQ ID NO: 711) was double-digested by EcoRV and XhoI, and a linearized vector was recovered by agarose gel electrophoresis. Using the prepared pCDH-CMV-EGFP-reporter3-EF1-Puro plasmid (SEQ ID NO: 712) as a template, were used to the DNA fragment containing the coding sequence of the Puro resistance gene was obtained by PCR amplification with primers Puro-PF1 (SEQ ID NO: 714) and Puro-PR1 (SEQ ID NO: 715). The DNA fragment was inserted into the pLVX-EF1a-BSD vector digested with enzymes by homologous recombination (NEB™, Gibson Assembly® Master Mix) to construct a recombinant vector pLVX-7NN-Puro library plasmid containing the 7NN random sequence (SEQ ID NO: 713). The reaction solution was transformed into Stbl3 competent cells, coated on an LB plate containing ampicillin, and cultured overnight at 37° C. All colonies were scraped for extracting plasmids.

### 2. Construction of Bacterial Expression Plasmid P15A-C12-279 of C12-279 Protein and Preparation of Competent Cells Containing the Plasmid

a. Construction of Bacterial Expression Plasmid of C12-279 Protein

A P15A-Cas-NC03 vector plasmid (SEQ ID NO: 716) was digested with SalI, and the linearized vector was recovered by agarose gel electrophoresis. Using the prepared pXC12-279-GFPPAM-DR5 plasmid (SEQ ID NO: 697) was used as a template, the DNA fragment encoding the C12-279 protein and the fusion fragment expressing the sgRNA were obtained by PCR with the primers ChkNLS-PF1 (SEQ ID NO: 718) and the synthesized C12-279-sgRNA fragment (SEQ ID NO: 717). The fragments were inserted into the P15A-Cas-NC03 vector digested with enzymes by homologous recombination (NEB™, Gibson Assembly® Master Mix) to construct the recombinant vector P15A-C12-279 (SEQ ID NO: 719). The reaction solution was transformed into Stbl3 competent cells, coated on the LB plate containing chloramphenicol, and cultured overnight at 37° C., and a single clone was picked for sequencing verification.

b. Preparation of Competent Cells Containing Bacterial Expression Plasmid P15A-C12-279

The P15A-C12-279 plasmid verified to be correct by sequencing was transformed into DH5a competent cells (Vidyabiotech™ CAT #: DL1001), and the single clone was picked and inoculated into LB medium containing chloramphenicol and cultured at 37° C. overnight.

Electrocompetent cells were prepared as follows.

The culture bacterial solution was inoculated into 100 mL of fresh LB medium containing chloramphenicol at a ratio of 1:100 for amplification culture at 37° C. and 220 rpm.

Then the culture bacterial solution was cultured until OD600 reached 0.5, the bacterial solution was transferred to a 50 mL centrifuge tube and pre-cooled on ice for 30 min.

Then the culture bacterial solution was centrifuged at 4000 rpm and 4° C. for 10 min, the cells were collected and resuspend in an equal volume of pre-cooled sterile water.

The above operations were repeated.

The cells were resuspended in 1/10 volume of pre-cooled sterile water containing 10% glycerol, divided into 50 uL/tube, and stored at −80° C. to obtain the P15A-C12-279 competent cells.

c. Performing Plasmid Elimination to Identify the PAM Sequence of the C12-279 Protein

100 ng of pLVX-7NN-Puro library plasmid was electroporated into the prepared P15A-C12-279 competent cells and DH5a electroporated cells, respectively, and marked as Lib1 (electroporated P15A-C12-279 competent cells) and Lib2 (electroporated DH5a competent cells), respectively.

After electroporation, 10 mL of LB medium was added, and the cells were revived and cultured at 37° C. and 220 rpm for 2 h.

The revived bacterial solution was centrifuged at 4000 rpm for 2 min to collect the bacteria, and then resuspended in 400 μL LB and coated on the LB plate. The electroporated DH5a bacterial solution was coated on the LB plate containing ampicillin, and the electroporated P15A-C12-279 competent cells were coated on the LB plate containing chloramphenicol and ampicillin and cultured at 37° C. overnight.

The bacterial cells were scraped from the culture plate and the plasmid DNA was extracted by alkaline lysis manner.

100 ng of each of the two extracted plasmid DNA samples was used as the PCR template, and a PCR amplification was performed using primers SiteSeq-PF1 (SEQ ID NO: 720) and SiteSeqPuro-PR (SEQ ID NO: 721). The obtained fragments were subjected to amplicon library construction using an NGS library construction kit (Xunshi Biotechnology, SynplSeq DNA Library Prep Kit for Illumina®), followed by NGS sequencing.

The NGS sequencing data from Lib1 and Lib2 cells is compared and analyzed, as shown in FIG. 7. It is proved that the PAM sequence recognized by the C12-279 is 5′-TTN-3′.

### Example 5. Cleavage Activity of C12-279 Protein on Target Nucleic Acid in 293T Cells

In this example, sgRNA targeting TTR genes in HEK293T cells was first designed and constructed into a pXC12-279-GFPPAM-DR5 plasmid (SEQ ID NO: 697) to obtain a plasmid pXC12-279-TTR01 targeting the TTR genes. After transfecting the HEK293T cells, an indel ratio was verified by NGS to verify the cleavage activity of the C12-279 protein in 293T cells. The specific operations were as follows.

1. Construction of sgRNA Plasmid

An sgRNA target site catgagcatgcagaggtgagtat (SEQ ID NO: 722) was designed based on TTR gene sequence information in a HEK293T cell line.

TABLE 11
sgRNA annealing primer

| Plasmid name | Primer name | Primer sequence |
|---|---|---|
| pXC12-279-TTR01 | TTR01-PF1 | cattgacgccatgagcatgcagaggtgagtatTTTTTG |
| | | (SEQ ID NO: 723) |
| | TTR01-PR1 | gtaccAAAAAatactcacctctgcatgctcatggcgtc |
| | | (SEQ ID NO: 724) |

The annealing primers were designed according to the sgRNA target site information, and the plasmid pXC12-279-GFPPAM-DR5 (SEQ ID NO: 697) was digested with BsmBI (Thermo Scientific™, ER0451) and Acc65I (Thermo Scientific™, ER0901). The primers (Table 11) were annealed and then connected with the vector to obtain a pXC12-279-TTR01 expression clone.

### 2. Detection of TTR Gene Editing Efficiency

Plating: 293T cell lines were plated when a confluency reached 70-80%, and a count of cells seeded in a 24-well plate was 5*10\hat{ }5 cells/well.

Transfection: Transfection was performed 12-14 h after plating. 100 μL Opti-MEM™ reduced serum medium (Gibco, Thermo Fisher Scientific), 1.5 μL PEI (Yeasen Biotechnology™, Polyethylenimine Linear (PEI) MW25000), and 500 ng pXC12-279-TTR01 plasmid were added to each well of a 24-well plate, mixed, and added to 293T cells for cell transfection after placing at room temperature for 20 min. After overnight transfection, a fresh culture medium was replaced, and culture was continued.

DNA extraction, PCR amplification, and NGS library construction: After 72 h of culture, the cells were washed with PBS, and then 100 μL of cell lysis solution (Viagen™, DirectPCR® Lysis Reagent (Cell)) was added for lysis to obtain lysate containing genomic DNA. The region near the target sequence was amplified for the genomic DNA. The PCR product was subjected to the NGS library construction and sequencing, and the sequencing result was analyzed. The indel rate is higher than 14%, as shown in FIG. 8.

### Example 6. PAM Recognition of CI1062732

Compared with the C12-279 protein (SEQ ID NO: 696), the protein CI1062732 (SEQ ID NO: 46) lacks dozens of amino acid residues at the N-terminal.

A PAM sequence recognized by CI1062732 was identified using a manner substantially the same as that in Example 4. A gRNA containing a “false” DR sequence GTAATGCGTCTCCCATTGACGCC (SEQ ID NO: 529) was combined with CI1062732 to target a 7nt random sequence plasmid library in bacteria. The sequencing analysis (as shown in FIG. 9) indicates that a “false” PAM motif identified by CI1062732 is 5′-TTNC-3′.

Subsequently, by analyzing a secondary structure of the “false” DR sequence, it is hypothesized that the 3′-end C base of the identified “false” PAM motif TTNC might be caused by an extra C at the 3′ end of DR (FIG. 10). Therefore, in subsequent CI1062732 test and C12-279 test, the DR sequence is GTAATGCGTCTCCCATTGACGC (SEQ ID NO: 704).

### Example 7. Screening, Design, Preparation, and Testing of C12-101-07 Protein

(1) The C12-101-07 protein was ultimately obtained through screening and design using complex bioinformatics manners.

An amino acid sequence of the C12-101-07 protein is:

(SEQ ID NO: 52)
IFPTQRRNTMSGNVVRSYKSVLRPNIRKRELLDATFNWFDRAYKKFFDVF
VCLYGGVEHDTVHSALLKETTDPDLVCATMWFRVVPKSSCDGISAQEMIR
RFGVYAGHEPSAVAMSYLTGNFDDKKNHWIDCREKFVALARDMVVKPESL
SIDIKSMIEHKILPICSQDNWQAWNAISQLFGEGKKENKAEKAQIFVDVV
SMLSNNSVQTWEDYKEVISKATGCRTAGEINSKFGGRPGILSVDFSKDDT
GSLPKDFIERRIKDLSQKAKEKSAAYELPNRMRLRELIASVIGPYRLETW
SSVAQRACGDIRSKNSNNLLYASERFNRTKEIEEILSAKGDVARAQNILG
QFRSGEFNEFAVEKRHLGNLESLYNIWSKSDMDTGIEEYSSIHKDEYDRD
PIVDLYRHIYPHREVISAKNFIDAAVLNKLLRLNASRHVHPTVFGKTVIS
FSPKSSAYGRITPPSEIVRGRPAGSHGMIWLTMELFDDGKWIEHHLPFHN
SRYYEEVYCYQEGLPVKDEPRSPMFGYRVGNAIADTSKIDNRRRKASKQF
LRAQQNITHNVSFDENTAFSVVRNGEDFSITISSRIKASNAKSLMTIGDR
IMGIDQNQTAANTYSIWEVVGDGDAGSIQHGNLSLKRIEDGHITSIIRGR
GGNFDQLNYGGLSYAKFSEWRSARLGFISSLNAELASKMNWDWCCLYEWN
ARYASALRKIIYSHKGIDIERVFRREIEDFVEGVLRIGSLSSDALQCLTN
AKSLISSYFFLNGKKEPEEQRKFDQGLFDLSAKIDKKRVNKRQQKTKRIM
SSIMQIARDRGVSFIVVEGKLSTATKDNKSKANQKAIDWCARAVVENLEH
SCSLVGIKLVAIDPMNTSHLDPFVYVLKTSLGKEARFASVVPAKINARHM
KNFKSWSSLLAGNGKIKKTTDAIYVGAFQDFCREYGFVAEDISKMSQSEI
QDKLSSHQFVLVPQRGGRVYMSTHPVSSEAKKIVYAGRERWYNNADVVAA
VNITLRGCERLSASRKTASTR.

The recombinant vector C12-101-07-pET28a-01 (SEQ ID NO: 725) was constructed by a manner substantially the same as that in Example 2, which was expressed and purified to obtain the C12-101-07 recombinant protein, with an amino acid count of 1122 aa and a structure of His tag-NLS-C12-101-07-SV40 NLS-nucleoplasmin NLS. The purified recombinant protein was detected by SDS-PAGE electrophoresis, and the result is shown in FIG. 11.

(2) The in vitro cleavage of PAM library by the C12-101-07 protein was prepared using a manner substantially the same as that in Example 3 to grab the PAM.

In vitro transcription was used to synthesize sgRNA containing a specific guide sequence, the sequence of which is as follows:

>C12-101-07-sgRNA
(SEQ ID NO: 726)
5′-atcgcaacatctcagaaacccgtcctaagttgacggGTGAGCAAGGG
CGAGGAGCTGTTC-3′.
>C12-101-07-sgRNA-Rev
(SEQ ID NO: 727)
5′-atcgcaacatctcagaaacccgtcctaagttgacggCGCAATGATGA
TCTCCGAGCCGTTCC-3′.
The sgRNA scaffold sequence is:
(SEQ ID NO: 534)
5′-atcgcaacatctcagaaacccgtcctaagttgacgg-3′.

The C12-101-07 protein was combined with forward and reverse gRNAs respectively for editing.

The PAM motifs shown in FIGS. 12 and 13 were identified during the experiment.

It is demonstrated that the PAM sequence recognized by C12-101-07 is 5′-TTN-3′.

### Example 8. In Vivo Editing Activity of C12-101-09 Protein in Bacteria and Verification of PAM

A similar protein C12-101-09 was designed based on C12-101-07, with the sequence as follows:

>C12-101-09
(SEQ ID NO: 728)
IFPTQRRNTMSGNVVRSYKSVLRPNIRKRELLDATFNWFDRAYKKFFDVF
VCLYGGVEHDTVHSALLKETTDPDLVCATMWFRVVPKSSCDGISAQEMIR
RFGVYAGHEPSAVAMSYLTGNFDDKKNHWIDCREKFVALARDMVVKPESL
SIDIKSMIEHKILPICSQDNWQAWNAISQLFGEGKKENKAEKAQIFVDVV
SMLSNNSVQTWEDYKEVISKATGCRTAGEINSKFGGRPGILSVDFSKDDT
GSLPKDFIERRIKDLSQKAKEKSAAYELPNRMRLRELIASVIGPYRLETW
SSVAQRACGDIRSKNSNNLLYASERFNRTKEIEEILSAKGDVARAQSILG
QFRSGEFNEFAVEKRHLGNLESLYNIWSKSDMDTGIEEYSSIHKDEYDRD
PIVDLYRHIYPHREVISAKNFIDAAVLNKLLRLNASRHVHPTVFGKTVIS
FSPKSSAYGRITPPSEIVRGRPAGSHGMIWLTMELFDDGKWIEHHLPFHN
SRYYEEVYCYQEGLPVKDEPRSPMFGYRVGNAIADTSKIDNRRRKASKQF
LRAQQNITHNVSFDENTAFSVVRNGEDFSITISSRIKASNAKSLMTIGDR
IMGIDQNQTAANTYSIWEVVGDGDAGSIQHGNLSLKRIEDGHITSIIRGR
GGNFDQLNYGGLSYAKFSEWRSARLGFISSLNAELASKMNWDWCCLYEWN
ARYASALRKIIYSHKGIDIERVFRREIEDFVEGVLRIGSLSSDALQCLTN
AKSLISSYFFLNGKKEPEEQRKFDQGLFDLSAKIDKKRVNKRQQKTKRIM
SSIMQIARDRGVSFIVVEGKLSTATKDNKSKANQKAIDWCARAVVENLEH
SCSLVGIKLVAIDPMNTSHLDPFVYVLKTSLGKEARFASVVPAKINARHM
KNFKSWSSLLAGNGKIKKTTDAIYVGAFQDFCREYGFVAEDISKMSQSEI
QDKLSSHQFVLVPQRGGRVYMSTHPVSSEAKKIVYAGRERWYNNADVVAA
VNITLRGCERLSASRKTASTR.

The recombinant vector plasmid P15A-C12-101-09 (SEQ ID NO: 729) was constructed by conventional manners, and then tests were performed using manners substantially the same as those in Example 4.

The motif recognized by C12-101-09 was identified, as shown in FIG. 14.

It is demonstrated that the PAM sequence recognized by C12-101-09 is 5′-TTN-3′.

### Example 9. Construction of Cell Lines Containing Different PAM Reporter Systems

Stable cell lines were prepared by lentiviral infection. By constructing lentiviral expression plasmids containing different PAM sequences, 293T cells were infected after virus packaging to construct cell lines containing GFP reporter systems with different PAM sequences for subsequent mutation screening.

(1) Construction of Lentiviral Expression Plasmids for GFP Reporter Systems with Different PAM Sequences

In addition to the prepared plasmid pCDH-CMV-EGFP-reporter3-EF1-Puro (SEQ ID NO: 712), a plasmid library with a PAM composition of NAAN was simultaneously constructed. A specific construction scheme was as follows.

A mixed library of EGFP fragments containing a detection system was synthesized by gene synthesis, and digested with XbaI+NotI, then the digested fragments were ligated to the XbaI+NotI digested vector of the plasmid pCDH-CMV-EGFP-reporter3-EF1-Puro using T4 DNA ligase. The ligation products were transformed into Stbl3 cells, and cultured on ampicillin-containing plates at 37° C. overnight.

For the mixed fragment library, multiple clones were picked for sequencing and identification to obtain 16 plasmids with different sequence compositions (i.e., PAM sequences being AAAA, AAAT, AAAG, AAAC, TAAA, TAAT, TAAG, TAAC, GAAA, GAAT, GAAG, GAAC, CAAA, CAAT, CAAG, CAAC), and then the plasmids were mixed at equal mass to obtain a Plasmid Lib (Puro-NAAN-eGFP-Lib, SEQ ID NO: 730) with a PAM sequence composition of NAAN for subsequent lentiviral packaging and construction of stable cell line.

In the unedited reporter system, there is a base insertion that is not a multiple of 3 from a start codon to a normal GFP reading frame (pCDH-CMV-EGFP-reporter3-EF1-Puro has a 32 bp base insertion, and the Plasmid Lib has a 29 bp base insertion, as shown in FIG. 15), which causes the GFP normal reading frame to be interrupted, and GFP is not expressed. Then an sgRNA target site was set inside a GFP expression frame, and indel was generated by the editing of Cas12, having a probability to restore the normal GFP reading frame, so that GFP was expressed normally, and the higher an editing efficiency, the higher the probability of the generated indel to restore the normal GFP reading frame. The editing efficiency of the Cas12 protein was characterized by counting GFP-expressing cells using flow cytometry.

(2) Lentiviral Packaging of GFP Reporter System Plasmids with Different PAM Sequences

The constructed pCDH-CMV-EGFP-reporter3-EF1-Puro plasmid and Plasmid Lib were mixed with virus packaging auxiliary plasmids pMD2.G (Miaoling Biosciences) and psPAX2 (Miaoling Biosciences) at a molar ratio of 1:1:1, respectively, and then transfected into 293T cells using PEI. After 48 h of transfection, a culture supernatant was taken and filtered with 0.45 μm of a filter film to obtain two crude viruses, namely pCDH-CMV-EGFP-reporter3-EF1-Puro and Plasmid Lib.

(3) Construction of the Detection Cell Line by Infecting 293T Cells Using the Crude Viruses pCDH-CMV-EGFP-Reporter3-EF1-Puro and Plasmid Lib

293T cells were infected with 1/4 volume of crude viruses pCDH-CMV-EGFP-reporter3-EF1-Puro and Plasmid Lib in culture medium. After 48 h of infection, the medium was changed and 2 ug/mL of Puromycin was added for screening.

For the 293T cells infected with pCDH-CMV-EGFP-reporter3-EF1-Puro, the screened cells were subjected to monoclonal screening by limited dilution, and the screened monoclonal cell lines were the cell line used for detection (namely, Reporter3 cell line).

For the 293T cells infected with Plasmid Lib, the cell pool obtained after drug screening was the cell line used for detection (namely, NAAN cell line).

### Example 10. Design of C12-279 Protein Mutation and Detection of Editing Efficiency

a. Determination of Mutation Position

A 3D structure of C12-279 protein was predicted and simulated through bioinformatics analysis and AI manners. Possible binding, recognition and cleavage sites of DNA of C12-279 were identified in combination with the 3D structure. The molecular cloning point mutation manner was used to construct mutation clones for these sites.

First, the mutations shown in Table 12 were designed.

TABLE 12
Editing efficiency of different mutation
positions of C12-279 in NAAN cell line

| | | Editing efficiency in NAAN cell |
|---|---|---|
| Mutation | | line, expressed as a multiple of |
| position | Mutant vector | the editing efficiency of C12-279 |
| D352R | C12-279-GFPPAM-01 | 1.45 ±0.36 |
| K631R | C12-279-GFPPAM-02 | 0.99 ± 0.18 |
| A988R | C12-279-GFPPAM-03 | 1.24 ± 0.14 |
| G989R | C12-279-GFPPAM-04 | 1.23 ± 0.44 |
| Q186R | C12-279-GFPPAM-05 | 1.74 ± 0.38 |
| G194R | C12-279-GFPPAM-06 | 0.01 |
| N195R | C12-279-GFPPAM-07 | 1.64 |
| G196R | C12-279-GFPPAM-08 | 0.31 ± 0.12 |
| G197R | C12-279-GFPPAM-09 | 1.44 ± 0.45 |
| N245R | C12-279-GFPPAM-10 | 0.46 ± 0.06 |
| L260R | C12-279-GFPPAM-11 | 1.56 ± 0.07 |
| A355R | C12-279-GFPPAM-12 | 1.31 ± 0.36 |
| C385R | C12-279-GFPPAM-13 | 1.20 ± 0.37 |
| P386R | C12-279-GFPPAM-14 | 1.40 ± 0.48 |
| H387R | C12-279-GFPPAM-15 | 0.92 ± 0.33 |
| G390R | C12-279-GFPPAM-16 | 0.00 |
| K391R | C12-279-GFPPAM-17 | 1.14 ± 0.34 |
| N392R | C12-279-GFPPAM-18 | 1.39 ± 0.18 |
| D429R | C12-279-GFPPAM-19 | 0.34 ± 0.06 |
| Q461R | C12-279-GFPPAM-20 | 0.99 ± 0.3 |
| Q462R | C12-280-GFPPAM-21 | 1.36 ± 0.39 |
| E485R | C12-280-GFPPAM-22 | 1.57 ± 0.37 |
| L611R | C12-280-GFPPAM-23 | 0.43 ± 0.09 |
| Q990R | C12-280-GFPPAM-24 | 0.94 ± 0.09 |
| A1136R | C12-280-GFPPAM-25 | Not detected |
| K1138R | C12-280-GFPPAM-26 | Not detected |
| T1139R | C12-280-GFPPAM-27 | Not detected |
| N/A | NAAN cell line | 0.00 |
| N/A | PEI-control | 0.00 |
| N/A | C12-279-GFPPAM-DR5 | 1.00 ± 0.28 |

b. Construction of Mutation Clones

After a specific mutation position was determined, the mutation base was introduced through primers to construct expression clones containing different mutation positions. The following was an example of the construction of a D352R mutation clone.

TABLE 13
Primer sequences for the construction of mutation clone
C12-279-GFPPAM-01 of C12-279

| Primer name | Primer sequences |
|---|---|
| ChkCas12-PF1 | CCAAGCTGGCTAGCGTTTAAACTTAAG (SEQ ID NO: 731) |
| ChkCas12-PR1 | ATGATCTCCGAGCCGTTTTTGGTACC (SEQ ID NO: 732) |
| C279-D352R-PF1 | GCTGAAGAGACACAGCagaATCGCCGCCGCT (SEQ ID NO: |
| | 733) (underlined being introduced amino acid mutations) |
| C279-D352R-PR1 | GGGAAGCGGCGGCGATtctGCTGTGTCTCT (SEQ ID NO: |
| | 734) (underlined being introduced amino acid mutations) |

Primers were designed for the mutation position D352R (as shown in Table 13), and the mutation position was introduced by primers C279-D352R-PF1 and C279-D352R-PR1. Using pXC12-279-GFPPAM-DR5 (SEQ NO: 1) as a template, ChkCas12-PF1+C279-D352R-PR1 was subjected to PCR amplification (Yijin Bio, PC019, UltraHiPF™ DNA Polymerase Kit) to obtain fragment C12-279-D352R-F1, ChkCas12-PR1+C279-D352R-PF1 was subjected to PCR amplification to obtain fragment C12-279-D352R-F2, plasmid pXC12-279-GFPPAM-DR5 (SEQ ID NO: 697) was digested using HindIII+KpnI, and 5646 bp vector fragment was gel recovered (Guangzhou Meiji Biotechnology Co., Ltd., D2110, HiPure Gel Pure Micro Kit), and was recombined in vitro with fragments C12-279-D352R-F1 and C12-279-D352R-F2 (NEB™, E2611L, Gibson Assembly® Master Mix), and the mutation clone plasmid C12-279-GFPPAM-01 was obtained by heat-shock transformation of Escherichia coli.

c. Detection of the Editing Efficiency of Mutants in NAAN Reporter System

Plating: cells were plated when a confluence of the NAAN cell line reached 70-80%, and a number of cells seeded in a 24-well plate was 5*10\hat{ }5 cells/well.

Transfection: transfection was performed 12-14 h after plating. 1.5 μL PEI (Yeasen Biotechnology™, 40815ES03, Polyethylenimine Linear (PEI) MW25000) and 500 ng mutation clone plasmid were added to 100 μL Opti-MEM™ reduced serum medium (Gibco, Thermo Fisher Scientific) per well of the 24-well plate, mixed, and added to the NAAN cell line for cell transfection after placing at room temperature for 20 min. After overnight transfection, a fresh culture medium was replaced, and the culture was continued for 72 h, followed by flow cytometry. Editing efficiencies of different mutant clones were characterized by the proportion of GFP-positive cells.

The results are shown in Table 16 and FIG. 16. The editing efficiency of different C12-279 mutants in NAAN cells is expressed as a multiple of the editing efficiency of C12-279. Multiple mutants improve the editing efficiency.

d. According to the results of a first round of mutation, a portion of mutation positions that significantly improved the editing efficiency were combined, and multiple mutation position combinations were tried to further improve the editing efficiency. The designed mutants were shown in Table 14.

TABLE 14
Editing efficiency of different mutation position
combinations of C12-279 in NAAN cell line

| Mutation position 1 | Mutation position 2 | Vector name of the mutant |
|---|---|---|
| D352R | Q186R | C12-279-GFPPAM-28 |
| D352R | L260R | C12-279-GFPPAM-29 |
| D352R | A355R | C12-279-GFPPAM-30 |
| A355R | L260R | C12-279-GFPPAM-31 |
| P386R | C385R | C12-279-GFPPAM-32 |
| E485R | Q462R | C12-279-GFPPAM-33 |

The vector was constructed using essentially the same manner as described above, and the editing efficiency was tested in the NAAN cell line. The results are shown in FIG. 17.

e. Construction of Mutant Plasmid Targeting Fixed PAM Combination

As the PAM of the NAAN cell line was a mixed library, it may theoretically have a certain impact on an actual editing efficiency. To more intuitively and efficiently demonstrate the effect of mutation on the editing efficiency, based on a sequence between a start codon of the Reporter3 cell line and a GFP reading frame, a target site with a PAM sequence of TTG and a target sequence of CTCACCTCGCGACGCAATGATG (SEQ ID NO: 735) was selected for subsequent editing efficiency test.

### Construction of Mutation Clones Targeting Reporter3 Cell Line

Based on C12-279-GFPPAM-DR5, the sgRNA was changed to target CTCACCTCGCGACGCAATGATG (SEQ ID NO: 735), the synthesized primers pCDH-PF1: GTACCGAAAAACATCATTGCGTCGCGAGGTGAGGCGTC (SEQ ID NO: 754) and pCDH-PR1: CATGACGCCTCACCTCGCGACGCAATGATGTTTTTCG (SEQ ID NO: 755) were annealed. The plasmid C12-279-GFPPAM-DR5 was digested with Acc65I (Thermo Scientific™) and BsmBI (Thermo Scientific™) to recover the vector and then connected with an annealing product to obtain the mutation clone C12-279-pCDH targeting the Reporter3 cell line. The construction manner of the mutation clones targeting Reporter3 cell lines was the same as the manner described in this example, except that the vector plasmid needed to be changed from C12-279-GFPPAM-DR5 to C12-279-pCDH, and the primer ChkCas12-PRI needed to be replaced by ChkCas12-PR2: GCGACGCAATGATGTTTTTCGGTACC (SEQ ID NO: 736).

Editing efficiency of some selected mutants was tested in Reporter3 cell line using the same manner as described for the NAAN reporter system, except that the cell line was changed from NAAN cell line to Reporter3 cell line.

According to the results of the first round of mutation, the entire 3D structure was corrected and marked, and data of the first round was placed in a new model for predictive analysis. Finally, possible second round mutation positions were analyzed and predicted, and mutations and detections were performed. By analogy, the best mutation combination of C12-279 was determined through a plurality of rounds of mutations, selections, and iterations. Table 15 shows the editing efficiency of C12-279 mutants in the Reporter3 cell line. An absolute value (average) of the editing efficiency of the C12-279-pCDH group, i.e., the C12-279 protein, is 8.55%.

TABLE 15
Editing efficiency of C12-279 mutants in Reporter3 cell line
(Expressed as multiple of editing efficiency of C12-279)

| | | | | Multiple |
|---|---|---|---|---|
| | | | | of editing |
| Mutation | Mutation | Mutation | | efficiency |
| position 1 | position 2 | position 3 | Group | of C12-279 |
| N/A | N/A | N/A | PEI-control | 0.02 ± 0.01 |
| N/A | N/A | N/A | Reporter3 cell line | 0.03 ± 0.02 |
| N/A | N/A | N/A | C12-279-pCDH | 1.00 ± 0.18 |
| D352R | N/A | N/A | C12-279-pCDH-01 | 1.18 ± 0.22 |
| G989R | N/A | N/A | C12-279-pCDH-04 | 1.27 ± 0.2 |
| Q186R | N/A | N/A | C12-279-pCDH-05 | 1.60 ± 0.32 |
| L260R | N/A | N/A | C12-279-pCDH-11 | 0.66 ± 0.14 |
| A355R | N/A | N/A | C12-279-pCDH-12 | 0.96 ± 0.14 |
| E485R | N/A | N/A | C12-279-pCDH-22 | 1.15 ± 0.32 |
| D352R | Q186R | N/A | C12-279-pCDH-28 | 1.39 ± 0.25 |
| A355R | L260R | N/A | C12-279-pCDH-31 | 0.95 ± 0.22 |
| D352R | Q186R | 133R | C12-279-pCDH-34 | 1.51 ± 0.31 |
| D352R | Q186R | G184R | C12-279-pCDH-35 | 2.28 |
| D352R | Q186R | S185R | C12-279-pCDH-36 | 1.91 |
| D352R | Q186R | G256R | C12-279-pCDH-37 | 0.06 ± 0.01 |
| D352R | Q186R | Y278R | C12-279-pCDH-38 | 1.86 ± 0.06 |
| D352R | Q186R | S285R | C12-279-pCDH-39 | 1.87 ± 0.06 |
| D352R | Q186R | Y316R | C12-279-pCDH-40 | 1.12 ± 0.2 |
| D352R | Q186R | H350R | C12-279-pCDH-41 | 2.11 |
| D352R | Q186R | A356R | C12-279-pCDH-42 | 2.01 ± 0.18 |
| D352R | Q186R | Q469R | C12-279-pCDH-43 | 1.61 ± 0.22 |
| D352R | Q186R | S491R | C12-279-pCDH-44 | 1.41 ± 0.48 |
| D352R | Q186R | K521R | C12-279-pCDH-45 | 1.73 ± 0.07 |
| D352R | Q186R | P525R | C12-279-pCDH-46 | 1.40 ± 0.09 |
| D352R | Q186R | K629R | C12-279-pCDH-47 | 1.74 ± 0.09 |
| D352R | Q186R | N633R | C12-279-pCDH-48 | 1.84 ± 0.06 |
| D352R | Q186R | D841R | C12-279-pCDH-49 | 1.83 ± 0.00 |
| D352R | Q186R | N898R | C12-279-pCDH-50 | 1.69 ± 0.05 |
| D352R | Q186R | K987R | C12-279-pCDH-51 | 1.76 ± 0.07 |
| D352R | Q186R | T991R | C12-279-pCDH-52 | 1.32 |
| D352R | Q186R | D1010R | C12-279-pCDH-53 | 1.58 ± 0.29 |
| D352R | Q186R | E1013R | C12-279-pCDH-54 | 2.00 ± 0.04 |

### Example 11. Design of C12-101-07 Protein Mutants and Detection of Editing Efficiency

Mutants shown in Table 16 were designed for the C12-101-07 protein.

The vector plasmid of mutant was constructed using a manner basically the same as that in Example 10, and the editing efficiency was detected.

a. Detection of the editing efficiency of single point mutation based on NAAN reporter system.

For selected mutation positions, C12-101-07-GFPPAM (SEQ ID NO: 737) was used as a cloning template plasmid and a control plasmid for transfection test, and the mutation clone was constructed by molecular cloning point mutation, and the editing efficiency was detected. The results are shown in Table 16. The editing efficiency of different C12-101-07 mutants in NAAN cell line is expressed as a multiple of the editing efficiency of C12-101-07. An absolute value (average) of the editing efficiencies of the C12-101-07-GFPPAM group, i.e., the C12-101-07 protein, is 0.23%.

TABLE 16
Different mutants of C12-101-07

| | | Editing efficiency |
|---|---|---|
| | | in NAAN cell line, |
| | | expressed as multiple |
| | | of the editing |
| Mutation | | efficiency of |
| position | Mutation vector | C12-101-07 |
| Q172R | C12-101-07-GFPPAM-01 | 2.08 ± 1.05 |
| G182R | C12-101-07-GFPPAM-02 | 0.01 ± 0.02 |
| E183R | C12-101-07-GFPPAM-03 | 0.95 ± 0.56 |
| G184R | C12-101-07-GFPPAM-04 | 0.02 ± 0.04 |
| K185R | C12-101-07-GFPPAM-05 | 1.01 ± 0.47 |
| K186R | C12-101-07-GFPPAM-06 | 0.02 ± 0.02 |
| V243R | C12-101-07-GFPPAM-07 | 0.71 ± 0.27 |
| L297R | C12-101-07-GFPPAM-08 | 0.04 ± 0.00 |
| N317R | C12-101-07-GFPPAM-09 | 0.52 ± 0.12 |
| E363R | C12-101-07-GFPPAM-10 | 0.19 ± 0.12 |
| H366R | C12-101-07-GFPPAM-11 | 0.02 ± 0.02 |
| V426R | C12-101-07-GFPPAM-12 | 1.15 ± 0.59 |
| K429R | C12-101-07-GFPPAM-13 | 0.80 ± 0.36 |
| L433R | C12-101-07-GFPPAM-14 | 0.57 ± 0.28 |
| S452R | C12-101-07-GFPPAM-15 | 1.09 ± 0.26 |
| S455R | C12-101-07-GFPPAM-16 | 0.07 ± 0.02 |
| K918R | C12-101-07-GFPPAM-17 | 0.50 ± 0.11 |
| T919R | C12-101-07-GFPPAM-18 | 0.29 ± 0.15 |
| T920R | C12-101-07-GFPPAM-19 | 1.21 ± 0.70 |
| A922R | C12-101-07-GFPPAM-20 | 1.18 ± 0.41 |
| N/A | C12-101-07-GFPPAM | 1.00 ± 0.67 |

b. The mutation positions that significantly improved the editing efficiency in the first round of mutations were combined to try two-point mutations. The mutants shown in Table 17 were designed.

Mutation clones were constructed using a point mutation molecular cloning method and the editing efficiency was determined based on the NAAN cell line.

The results are shown in Table 17. The editing efficiency of different C12-101-07 mutants in the NAAN cell line is expressed as the multiple of the editing efficiency of C12-101-07.

TABLE 17
C12-101-07 mutants

| | | | Editing efficiency in NAAN |
|---|---|---|---|
| | | | cell line, expressed as |
| Mutation | Mutation | | multiple of the editing |
| position 1 | position 2 | Mutation vector | efficiency of C12-101-07 |
| Q172R | V426R | pXC12-101-07- | 2.15 ± 1.05 |
| | | TwoMut-01 | |
| Q172R | S452R | pXC12-101-07- | 1.89 ± 0.68 |
| | | TwoMut-02 | |
| Q172R | T920R | pXC12-101-07- | 4.04 ± 1.00 |
| | | TwoMut-03 | |
| V426R | S452R | pXC12-101-07- | 1.56 ± 0.52 |
| | | TwoMut-04 | |
| V426R | T920R | pXC12-101-07- | 2.11 ± 0.79 |
| | | TwoMut-05 | |
| S452R | T920R | pXC12-101-07- | 1.19 ± 0.21 |
| | | TwoMut-06 | |
| N/A | N/A | pXC12-101-07- | 1.00 ± 0.37 |
| | | GFPPAM | |

c. Construction of Mutation Clones Targeting Reporter3 Cell Lines and Detection of the Editing Efficiency

Using a manner basically the same as that of Example 10, the target sequence on the original C12-101-07-GFPPAM vector was replaced. The sgRNA target site for the NAAN cell line was replaced with a site targeting the Reporter3 cell line, resulting in the control plasmid 101-07-sgRNA02. Then, the mutant vector was constructed, and the editing efficiency was tested in the Reporter3 cell line.

The results are shown in Table 18. The editing efficiency of different C12-101-07 mutations in the Reporter3 cell line is expressed as a multiple of the editing efficiency of C12-101-07. The absolute value (average) of the editing efficiency of the 101-07-sgRNA02 control group, i.e., the C12-101-07 protein, is 10.32%.

TABLE 18
Editing efficiency of different mutants of C12-101-07 in Reporter3 cell
lines (Expressed as multiple of editing efficiency of C12-101-07)

| | | | Editing efficiency, |
|---|---|---|---|
| | | | expressed as multiple of |
| Mutation | Mutation | | the editing efficiency of |
| position 1 | position 2 | Vector | C12-101-07 |
| V15R | N/A | pXC12-101-07-21 | 1.67 ± 0.04 |
| A173W | N/A | pXC12-101-07-22 | 0.1 ± 0.01 |
| G239R | N/A | pXC12-101-07-23 | 0.04 ± 0.00 |
| D264R | N/A | pXC12-101-07-24 | 1.28 ± 0.16 |
| E271R | N/A | pXC12-101-07-25 | 0.96 ± 0.1 |
| Y295R | N/A | pXC12-101-07-26 | 0.53 ± 0.03 |
| T329R | N/A | pXC12-101-07-27 | 1.3 ± 0.17 |
| E331R | N/A | pXC12-101-07-28 | 1.06 ± 0.01 |
| I335R | N/A | pXC12-101-07-29 | 1.52 ± 0.16 |
| S430R | N/A | pXC12-101-07-30 | 0.79 ± 0.15 |
| S465R | N/A | pXC12-101-07-31 | 1.18 ± 0.37 |
| E493R | N/A | pXC12-101-07-32 | 1.61 ± 0.02 |
| P497R | N/A | pXC12-101-07-33 | 0.15 ± 0.04 |
| K587R | N/A | pXC12-101-07-34 | 0.3 ± 0.01 |
| E768R | N/A | pXC12-101-07-35 | 1.4 ± 0.07 |
| T825R | N/A | pXC12-101-07-36 | 0.95 ± 0.04 |
| A911R | N/A | pXC12-101-07-37 | 1.33 ± 0.21 |
| G914R | N/A | pXC12-101-07-38 | 0.26 ± 0.05 |
| K915R | N/A | pXC12-101-07-39 | 1.03 ± 0.26 |
| I916R | N/A | pXC12-101-07-40 | 0.51 ± 0.12 |
| E940R | N/A | pXC12-101-07-42 | 1.13 ± 0.11 |
| N347E | N/A | pXC12-101-07-43 | 0.99 ± 0.13 |
| K339R | N/A | pXC12-101-07-44 | 1.13 ± 0.01 |
| N347E | K339R | pXC12-101-07-45 | 1.16 ± 0.08 |
| Q172R | V426R | pXC12-101-07-Tw | 2.020.06 |
| | | oMut-01 | |
| Q172R | S452R | pXC12-101-07-Tw | 1.72 ± 0.09 |
| | | oMut-02 | |
| Q172R | T920R | pXC12-101-07-Tw | 2.15 ± 0.12 |
| | | oMut-03 | |
| V426R | S452R | pXC12-101-07-Tw | 1.56 ± 0.12 |
| | | oMut-04 | |
| V426R | T920R | pXC12-101-07-Tw | Not detected |
| | | oMut-05 | |
| S452R | T920R | pXC12-101-07-Tw | Not detected |
| | | oMut-06 | |
| N/A | N/A | 101-07-sgRNA02 | 1.00 ± 0.11 |
| | | control | |

### Example 12. Construction of Different Mutants by Site-Directed Mutagenesis of C12-279 Protein

The amino acid sequence of C12-279 protein was SEQ ID NO: 696. Two mutation primers F/R were designed at the mutation position, and the required mutation sequence was introduced through the primers. Combined with the universal primers at both ends of the vector, PCR amplification was performed to obtain two mutation fragments F1 and F2. The mutation fragments F1 and F2 were homologously recombined with the linearized vector to obtain a mutation plasmid. In this example, single-point mutation (D426R, L860R) mutants and multi-point mutation (D426R&L860R) mutants were constructed using the two amino acid positions 426 and 860. The specific steps were as follows.

The primers for site-directed mutagenesis at positions 426 and 860 are shown in Table 19.

TABLE 19
Primers

| Primer name | Primer sequences |
|---|---|
| ChkCas12-PF1 | CCAAGCTGGCTAGCGTTTAAACTTAAG (SEQ ID NO: 731) |
| ChkCas12-PR2 | GCGACGCAATGATGTTTTTCGGTACC (SEQ ID NO: 736) |
| 279_426_F | gagttcaagAGAggcttcgacagagagc (underlined being introduced amino |
| | acid mutation) (SEQ ID NO: 756) |
| 279_426_R | tcgaagccTCTcttgaactcggcgcagtag (underlined being introduced |
| | amino acid mutation) (SEQ ID NO: 757) |
| 279_860_F | aaccAGGagacagaacaagggcgagg (underlined being introduced amino |
| | acid mutation) (SEQ ID NO: 758) |
| 279_860_R | ccttgttctgtctCCTggtttccagcttgttcag (underlined being introduced |
| | amino acid mutation) (SEQ ID NO: 759) |

### Construction of Single Point Mutation Clones at Positions 426 and 860

Using C12-279-pCDH plasmid as a template, ChkCas12-PF1+279_426_R was subjected to PCR amplification (Yijin Bio, PC019, UltraHiPF™ DNA Polymerase Kit) to obtain mutation fragment D426R-F1, and ChkCas12-PR2+279_426_F was subjected to PCR amplification to obtain mutation fragment D426R-F2. Plasmid C12-279-pCDH was digested with HindIII+KpnI, and 5647 bp vector fragment was gel recovered (Guangzhou Meiji Biotechnology Co., Ltd., D2110, HiPure Gel Pure Micro Kit). The 5647 bp vector fragment was recombined in vitro with fragments D426R-F1 and D426R-F2 (NEB™, E2611L, Gibson Assembly® Master Mix), and the plasmid C12-279-pCDH-426 with position 426 mutation was obtained by heat shock transformation of Escherichia coli.

The same steps and methods were used, except that primer 279_426_F was replaced with 279_860_F and primer 279_426_R was replaced with 279_860_R, and the same amplification and recombination were performed. The plasmid C12-279-pCDH-860 of the position 860 mutation was obtained by heat shock transformation of Escherichia coli. The construction method of the Cas protein mutation plasmids of other different positions was consistent with the method for the positions 426 and 860, except that the mutation primers for each different position needed to be designed.

### Verification of Editing Activity of Cas Protein Mutants

The test was conducted based on the Reporter3 cell line constructed in the above examples. The sequence between the start codon and the GFP reading frame was 32 bases, which was not an integer multiple of 3, resulting in abnormal reading frame of GFP and no expression of GFP. The Reporter3 cell line was edited using a target site whose PAM recognized by the Cas protein mutant was TTG and target sequence was CTCACCTCGCGACGCAATGATG (SEQ ID NO: 735), indels were generated, and a normal reading frame of GFP was restored. A flow cytometry was used to detect the proportion of cells that restored GFP expression to characterize the editing efficiency of different mutants. The specific operations were as follows.

Cell culture and plating: when the cell line was cultured to 70-80% confluence, the plating was performed, and the count of cells seeded in a 24-well plate was 5*10\hat{ }5 cells/well.

Transfection: the transfection was performed 12-14 h after plating. 1.5 μL PEI (Yeasen Biotechnology™) and 500 ng mutation plasmid were added to 100 μL Opti-MEM™ reduced serum medium (Gibco, Thermo Fisher Scientific) per well of the 24-well plate, mixed, and added to the Reporter3 cell line for cell transfection after placing at room temperature for 20 min. Fresh culture medium was replaced after overnight transfection and continued to be cultured for 72 h, the flow cytometry was used for detection. The editing efficiency of different mutation clones were characterized according to a GFP-positive cell proportion, and the average of a plurality of batches of data was taken. The specific results are shown in Table 20 and FIG. 18. An average absolute value of the editing efficiency of a wild-type C12-279 group is 9.0%.

TABLE 20
Editing efficiency of each mutant of C12-279 in Reporter3 cell
line (Expressed as multiple of editing efficiency of C12-279)

| | Editing |
|---|---|
| | efficiency |
| | (expressed as |
| | multiple of |
| | the editing |
| | efficiency of |
| Group | C12-279) |
| Wild-type C12-279 | 1.00 |
| Mut-01 (Q186R) | 1.58 |
| Mut-02 | 1.62 |
| (Q186 & D352R) | |
| Mut-03 | 1.87 |
| (G184R & Q186R | |
| & D352R) | |
| Cell Line NC | 0.05 |
| negative control | |
| PEI NC | 0.08 |
| negative control | |
| 1R | 2.07 |
| 2R | 1.79 |
| 3R | 2.05 |
| 4R | 1.53 |
| 5R | 2.00 |
| 6R | 0.98 |
| 7R | 1.91 |
| 8R | 0.81 |
| 9R | 0.82 |
| 10R | 1.60 |
| 12R | 0.10 |
| 13R | 0.91 |
| 14R | 1.13 |
| 15R | 1.32 |
| 16R | 1.37 |
| 19R | 1.26 |
| 21R | 1.29 |
| 22R | 1.19 |
| 23R | 1.07 |
| 24R | 1.56 |
| 25R | 1.05 |
| 26R | 1.18 |
| 27R | 1.33 |
| 28R | 0.51 |
| 29R | 0.08 |
| 30R | 1.59 |
| 32R | 1.30 |
| 33R | 0.80 |
| 34R | 1.08 |
| 35R | 0.03 |
| 36R | 0.07 |
| 37R | 0.94 |
| 38R | 0.17 |
| 39R | 1.23 |
| 40R | 0.08 |
| 41R | 0.52 |
| 42R | 0.45 |
| 43R | 1.14 |
| 44R | 0.93 |
| 46R | 1.10 |
| 47R | 1.34 |
| 48R | 1.43 |
| 49R | 1.17 |
| 50R | 0.20 |
| 51R | 1.46 |
| 53R | 0.05 |
| 54R | 0.42 |
| 55R | 1.40 |
| 56R | 0.28 |
| 57R | 0.07 |
| 58R | 1.42 |
| 59R | 1.51 |
| 60R | 0.05 |
| 61R | 0.84 |
| 62R | 0.71 |
| 64R | 0.06 |
| 65R | 0.19 |
| 66R | 1.42 |
| 67R | 0.47 |
| 68R | 0.12 |
| 69R | 0.65 |
| 70R | 0.98 |
| 71R | 0.06 |
| 72R | 0.08 |
| 73R | 0.03 |
| 74R | 0.44 |
| 75R | 0.05 |
| 76R | 1.12 |
| 77R | 0.72 |
| 78R | 1.28 |
| 79R | 0.64 |
| 80R | 0.20 |
| 81R | 1.19 |
| 82R | 0.99 |
| 83R | 0.89 |
| 84R | 0.97 |
| 85R | 1.35 |
| 86R | 1.01 |
| 88R | 0.13 |
| 89R | 1.34 |
| 90R | 1.30 |
| 91R | 0.84 |
| 92R | 0.81 |
| 93R | 0.27 |
| 94R | 0.07 |
| 95R | 0.10 |
| 96R | 0.05 |
| 97R | 0.09 |
| 98R | 0.12 |
| 99R | 0.07 |
| 101R | 0.39 |
| 102R | 0.98 |
| 103R | 1.25 |
| 104R | 1.00 |
| 105R | 1.13 |
| 106R | 0.33 |
| 108R | 1.41 |
| 109R | 0.81 |
| 110R | 1.03 |
| 111R | 0.61 |
| 112R | 0.78 |
| 114R | 1.25 |
| 115R | 1.06 |
| 116R | 0.95 |
| 117R | 0.98 |
| 118R | 1.41 |
| 119R | 0.80 |
| 120R | 0.29 |
| 121R | 1.35 |
| 123R | 1.34 |
| 124R | 1.29 |
| 125R | 1.33 |
| 126R | 0.93 |
| 127R | 1.20 |
| 128R | 1.31 |
| 129R | 1.30 |
| 130R | 0.97 |
| 131R | 1.00 |
| 132R | 0.43 |
| 133R | 0.92 |
| 134R | 0.98 |
| 135R | 0.12 |
| 136R | 0.39 |
| 137R | 0.09 |
| 138R | 1.43 |
| 139R | 1.37 |
| 140R | 1.26 |
| 141R | 1.60 |
| 142R | 1.02 |
| 143R | 1.20 |
| 144R | 1.11 |
| 145R | 0.95 |
| 146R | 1.16 |
| 147R | 0.92 |
| 148R | 0.10 |
| 149R | 0.10 |
| 151R | 1.06 |
| 152R | 1.20 |
| 153R | 0.08 |
| 154R | 0.97 |
| 155R | 1.20 |
| 156R | 0.16 |
| 157R | 0.99 |
| 158R | 0.85 |
| 159R | 1.09 |
| 160R | 0.12 |
| 161R | 1.23 |
| 162R | 0.57 |
| 163R | 0.31 |
| 164R | 0.09 |
| 165R | 0.92 |
| 166R | 0.95 |
| 167R | 0.08 |
| 169R | 1.15 |
| 170R | 0.47 |
| 171R | 0.07 |
| 172R | 0.10 |
| 174R | 0.09 |
| 175R | 1.40 |
| 176R | 0.16 |
| 177R | 0.10 |
| 178R | 1.60 |
| 179R | 0.19 |
| 180R | 0.34 |
| 182R | 1.09 |
| 183R | 1.08 |
| 184R | 1.22 |
| 185R | 1.41 |
| 186R | 1.23 |
| 187R | 0.30 |
| 188R | 0.39 |
| 189R | 0.11 |
| 190R | 0.08 |
| 191R | 1.01 |
| 192R | 0.04 |
| 194R | 0.10 |
| 195R | 1.22 |
| 196R | 0.40 |
| 197R | 1.34 |
| 198R | 0.19 |
| 199R | 0.07 |
| 200R | 1.06 |
| 202R | 0.19 |
| 203R | 1.31 |
| 204R | 0.03 |
| 205R | 0.60 |
| 206R | 1.07 |
| 207R | 0.06 |
| 208R | 0.04 |
| 209R | 0.89 |
| 210R | 0.91 |
| 211R | 0.05 |
| 212R | 0.49 |
| 213R | 1.24 |
| 214R | 1.02 |
| 215R | 0.06 |
| 216R | 1.08 |
| 217R | 1.10 |
| 218R | 0.94 |
| 219R | 0.97 |
| 220R | 1.07 |
| 221R | 0.95 |
| 222R | 0.67 |
| 223R | 1.27 |
| 224R | 1.06 |
| 225R | 0.72 |
| 227R | 0.45 |
| 228R | 0.06 |
| 229R | 0.70 |
| 230R | 0.47 |
| 231R | 0.39 |
| 232R | 0.05 |
| 233R | 0.59 |
| 234R | 1.38 |
| 235R | 0.51 |
| 236R | 0.06 |
| 237R | 0.99 |
| 238R | 0.05 |
| 239R | 1.10 |
| 240R | 0.36 |
| 242R | 0.59 |
| 243R | 0.45 |
| 244R | 0.05 |
| 245R | 0.41 |
| 247R | 0.70 |
| 248R | 0.04 |
| 249R | 0.12 |
| 250R | 1.29 |
| 251R | 0.86 |
| 252R | 1.18 |
| 253R | 0.06 |
| 255R | 0.30 |
| 256R | 0.05 |
| 257R | 1.41 |
| 258R | 0.06 |
| 259R | 0.36 |
| 260R | 0.99 |
| 261R | 0.07 |
| 262R | 0.09 |
| 263R | 0.69 |
| 265R | 0.87 |
| 266R | 0.96 |
| 267R | 0.80 |
| 268R | 1.18 |
| 269R | 1.32 |
| 270R | 0.11 |
| 271R | 0.93 |
| 272R | 0.76 |
| 273R | 0.77 |
| 274R | 1.14 |
| 275R | 0.10 |
| 276R | 1.20 |
| 278R | 0.60 |
| 279R | 0.12 |
| 281R | 1.03 |
| 282R | 0.09 |
| 283R | 0.13 |
| 284R | 0.93 |
| 285R | 0.93 |
| 286R | 0.05 |
| 287R | 1.08 |
| 288R | 0.87 |
| 289R | 0.68 |
| 290R | 0.76 |
| 291R | 0.83 |
| 292R | 0.09 |
| 293R | 0.95 |
| 294R | 0.11 |
| 295R | 1.18 |
| 296R | 1.12 |
| 297R | 1.07 |
| 298R | 0.04 |
| 299R | 1.26 |
| 300R | 0.04 |
| 301R | 0.43 |
| 302R | 0.05 |
| 303R | 1.21 |
| 305R | 1.07 |
| 306R | 0.21 |
| 308R | 1.22 |
| 309R | 1.26 |
| 310R | 0.80 |
| 313R | 0.94 |
| 315R | 1.15 |
| 316R | 0.49 |
| 317R | 0.38 |
| 318R | 1.06 |
| 319R | 0.54 |
| 320R | 0.05 |
| 321R | 0.23 |
| 322R | 1.29 |
| 323R | 0.55 |
| 324R | 0.07 |
| 325R | 0.44 |
| 327R | 1.19 |
| 328R | 0.08 |
| 329R | 1.04 |
| 330R | 0.29 |
| 331R | 0.21 |
| 332R | 0.04 |
| 333R | 1.67 |
| 334R | 0.68 |
| 335R | 0.77 |
| 336R | 0.04 |
| 337R | 0.88 |
| 339R | 0.06 |
| 340R | 0.91 |
| 341R | 0.99 |
| 342R | 0.60 |
| 343R | 0.12 |
| 344R | 0.78 |
| 345R | 0.70 |
| 346R | 0.92 |
| 347R | 1.37 |
| 348R | 1.28 |
| 350R | 1.19 |
| 351R | 0.97 |
| 352R | 1.15 |
| 353R | 1.15 |
| 354R | 1.15 |
| 355R | 1.13 |
| 356R | 1.56 |
| 357R | 1.03 |
| 358R | 1.00 |
| 359R | 1.03 |
| 360R | 1.12 |
| 361R | 1.25 |
| 362R | 0.96 |
| 363R | 1.13 |
| 364R | 0.81 |
| 365R | 0.84 |
| 366R | 0.03 |
| 367R | 0.18 |
| 368R | 1.00 |
| 369R | 1.29 |
| 370R | 0.42 |
| 371R | 1.18 |
| 372R | 1.06 |
| 373R | 0.04 |
| 374R | 0.02 |
| 375R | 1.40 |
| 376R | 1.60 |
| 377R | 1.20 |
| 378R | 1.41 |
| 379R | 1.57 |
| 380R | 1.07 |
| 381R | 1.05 |
| 382R | 1.01 |
| 383R | 1.51 |
| 384R | 0.04 |
| 385R | 1.20 |
| 386R | 1.09 |
| 387R | 1.14 |
| 388R | 0.99 |
| 389R | 0.06 |
| 390R | 0.26 |
| 391R | 1.05 |
| 392R | 1.15 |
| 393R | 0.04 |
| 394R | 0.87 |
| 395R | 0.10 |
| 396R | 1.28 |
| 397R | 1.40 |
| 398R | 0.99 |
| 399R | 1.01 |
| 400R | 1.62 |
| 401R | 1.03 |
| 402R | 1.21 |
| 403R | 1.20 |
| 404R | 1.02 |
| 405R | 1.33 |
| 406R | 1.13 |
| 407R | 1.07 |
| 408R | 1.38 |
| 409R | 1.09 |
| 410R | 0.81 |
| 411R | 1.20 |
| 412R | 1.21 |
| 413R | 1.01 |
| 414R | 0.91 |
| 415R | 0.03 |
| 416R | 1.48 |
| 417R | 0.92 |
| 418R | 1.30 |
| 419R | 0.87 |
| 420R | 1.10 |
| 421R | 1.17 |
| 422R | 1.19 |
| 423R | 0.79 |
| 424R | 1.12 |
| 425R | 1.24 |
| 426R | 2.22 |
| 427R | 0.68 |
| 428R | 0.44 |
| 429R | 0.74 |
| 431R | 1.15 |
| 432R | 0.05 |
| 433R | 0.91 |
| 435R | 1.29 |
| 436R | 0.04 |
| 437R | 0.29 |
| 439R | 0.50 |
| 440R | 0.04 |
| 441R | 1.18 |
| 442R | 1.06 |
| 443R | 1.44 |
| 444R | 1.10 |
| 446R | 1.24 |
| 447R | 1.23 |
| 448R | 1.12 |
| 449R | 1.46 |
| 450R | 1.27 |
| 451R | 0.97 |
| 452R | 1.04 |
| 453R | 0.04 |
| 454R | 1.29 |
| 455R | 1.04 |
| 456R | 1.45 |
| 457R | 0.11 |
| 458R | 0.03 |
| 459R | 1.53 |
| 460R | 0.03 |
| 461R | 1.11 |
| 462R | 1.58 |
| 463R | 1.05 |
| 464R | 0.81 |
| 467R | 1.22 |
| 469R | 1.46 |
| 470R | 1.03 |
| 471R | 0.10 |
| 472R | 0.10 |
| 473R | 1.19 |
| 474R | 0.03 |
| 475R | 1.28 |
| 476R | 0.99 |
| 477R | 0.20 |
| 478R | 1.13 |
| 479R | 0.48 |
| 480R | 1.03 |
| 481R | 0.46 |
| 482R | 1.30 |
| 483R | 0.41 |
| 484R | 1.68 |
| 485R | 1.52 |
| 486R | 0.47 |
| 487R | 0.22 |
| 488R | 0.91 |
| 489R | 1.16 |
| 490R | 0.77 |
| 491R | 1.12 |
| 492R | 1.04 |
| 493R | 0.82 |
| 494R | 1.31 |
| 496R | 1.18 |
| 497R | 0.75 |
| 499R | 1.07 |
| 500R | 0.94 |
| 501R | 1.33 |
| 502R | 1.02 |
| 503R | 0.61 |
| 504R | 0.56 |
| 506R | 0.10 |
| 507R | 0.93 |
| 508R | 0.05 |
| 509R | 1.47 |
| 510R | 0.07 |
| 511R | 0.85 |
| 512R | 0.60 |
| 513R | 0.22 |
| 514R | 1.10 |
| 515R | 1.36 |
| 516R | 0.85 |
| 517R | 0.62 |
| 518R | 1.11 |
| 519R | 0.09 |
| 520R | 0.86 |
| 521R | 0.90 |
| 522R | 1.17 |
| 523R | 0.10 |
| 524R | 0.33 |
| 525R | 0.89 |
| 526R | 0.08 |
| 527R | 1.00 |
| 528R | 0.04 |
| 529R | 0.38 |
| 531R | 0.07 |
| 532R | 0.42 |
| 533R | 1.07 |
| 534R | 0.09 |
| 535R | 0.31 |
| 536R | 0.39 |
| 537R | 0.62 |
| 538R | 1.02 |
| 539R | 0.83 |
| 540R | 1.04 |
| 541R | 0.91 |
| 542R | 1.21 |
| 543R | 0.12 |
| 544R | 0.05 |
| 545R | 0.81 |
| 546R | 1.11 |
| 547R | 0.72 |
| 548R | 1.02 |
| 549R | 1.21 |
| 550R | 0.08 |
| 552R | 0.68 |
| 553R | 1.23 |
| 555R | 0.35 |
| 556R | 1.17 |
| 557R | 0.79 |
| 558R | 1.37 |
| 559R | 1.17 |
| 560R | 0.92 |
| 561R | 1.41 |
| 562R | 1.08 |
| 563R | 1.12 |
| 564R | 1.08 |
| 565R | 1.23 |
| 566R | 1.07 |
| 567R | 1.25 |
| 568R | 1.34 |
| 569R | 0.67 |
| 570R | 0.07 |
| 571R | 0.07 |
| 572R | 1.29 |
| 573R | 0.10 |
| 574R | 1.16 |
| 575R | 0.85 |
| 576R | 0.67 |
| 577R | 1.28 |
| 578R | 1.15 |
| 579R | 0.96 |
| 580R | 1.38 |
| 581R | 1.19 |
| 582R | 1.16 |
| 583R | 0.72 |
| 584R | 1.18 |
| 585R | 1.31 |
| 586R | 1.12 |
| 587R | 0.73 |
| 589R | 1.01 |
| 590R | 1.13 |
| 592R | 0.10 |
| 593R | 1.20 |
| 594R | 0.09 |
| 595R | 1.16 |
| 596R | 0.07 |
| 597R | 1.45 |
| 598R | 1.22 |
| 599R | 1.23 |
| 601R | 0.25 |
| 602R | 0.74 |
| 603R | 1.01 |
| 604R | 0.79 |
| 605R | 1.26 |
| 606R | 1.13 |
| 607R | 1.52 |
| 608R | 0.82 |
| 609R | 1.45 |
| 610R | 1.15 |
| 611R | 0.68 |
| 612R | 1.20 |
| 613R | 0.07 |
| 614R | 0.79 |
| 615R | 0.04 |
| 616R | 0.06 |
| 618R | 1.35 |
| 619R | 0.09 |
| 620R | 0.77 |
| 621R | 1.28 |
| 622R | 0.05 |
| 623R | 1.44 |
| 624R | 0.05 |
| 625R | 1.00 |
| 626R | 0.07 |
| 627R | 0.24 |
| 628R | 0.47 |
| 630R | 0.55 |
| 631R | 1.17 |
| 632R | 1.05 |
| 633R | 1.19 |
| 634R | 1.26 |
| 635R | 1.18 |
| 636R | 1.38 |
| 637R | 1.17 |
| 638R | 1.69 |
| 639R | 1.55 |
| 640R | 1.55 |
| 641R | 0.95 |
| 642R | 1.30 |
| 643R | 1.14 |
| 644R | 0.69 |
| 645R | 0.44 |
| 646R | 1.24 |
| 647R | 0.53 |
| 648R | 0.04 |
| 649R | 0.12 |
| 650R | 0.04 |
| 651R | 0.04 |
| 652R | 0.04 |
| 653R | 0.37 |
| 654R | 0.56 |
| 655R | 0.69 |
| 656R | 0.83 |
| 657R | 1.10 |
| 658R | 0.07 |
| 659R | 0.14 |
| 660R | 0.54 |
| 661R | 0.10 |
| 662R | 0.75 |
| 663R | 0.09 |
| 664R | 1.06 |
| 665R | 1.17 |
| 666R | 1.04 |
| 667R | 1.06 |
| 668R | 1.10 |
| 669R | 1.09 |
| 670R | 1.25 |
| 671R | 1.06 |
| 672R | 1.16 |
| 673R | 1.20 |
| 674R | 1.22 |
| 675R | 1.21 |
| 676R | 0.21 |
| 678R | 1.19 |
| 679R | 1.20 |
| 680R | 1.01 |
| 681R | 0.12 |
| 683R | 0.46 |
| 684R | 0.10 |
| 685R | 1.02 |
| 686R | 0.05 |
| 688R | 0.39 |
| 689R | 0.04 |
| 691R | 0.13 |
| 692R | 0.86 |
| 693R | 0.10 |
| 694R | 1.20 |
| 695R | 0.54 |
| 696R | 1.32 |
| 697R | 1.70 |
| 698R | 0.90 |
| 699R | 1.37 |
| 700R | 0.84 |
| 701R | 0.70 |
| 702R | 0.11 |
| 703R | 0.12 |
| 704R | 0.08 |
| 705R | 0.53 |
| 706R | 0.35 |
| 707R | 0.49 |
| 708R | 0.12 |
| 709R | 0.51 |
| 710R | 1.14 |
| 711R | 0.77 |
| 712R | 1.35 |
| 713R | 0.90 |
| 715R | 1.17 |
| 716R | 1.19 |
| 717R | 0.69 |
| 719R | 1.23 |
| 720R | 0.86 |
| 721R | 1.24 |
| 722R | 1.42 |
| 723R | 1.08 |
| 724R | 1.14 |
| 725R | 1.24 |
| 727R | 1.32 |
| 728R | 1.26 |
| 729R | 1.21 |
| 730R | 1.20 |
| 731R | 1.44 |
| 732R | 1.39 |
| 733R | 1.49 |
| 734R | 1.34 |
| 736R | 0.92 |
| 737R | 1.22 |
| 738R | 1.05 |
| 739R | 1.08 |
| 740R | 1.17 |
| 741R | 1.16 |
| 742R | 1.27 |
| 743R | 0.99 |
| 744R | 0.08 |
| 745R | 1.33 |
| 746R | 1.16 |
| 747R | 1.03 |
| 748R | 1.19 |
| 749R | 1.01 |
| 751R | 1.39 |
| 752R | 0.95 |
| 753R | 1.01 |
| 754R | 1.24 |
| 755R | 1.42 |
| 756R | 1.18 |
| 758R | 1.41 |
| 759R | 1.13 |
| 760R | 1.39 |
| 761R | 0.19 |
| 762R | 0.25 |
| 764R | 0.64 |
| 765R | 0.91 |
| 766R | 1.18 |
| 767R | 1.34 |
| 768R | 1.36 |
| 769R | 0.24 |
| 771R | 1.63 |
| 772R | 0.11 |
| 773R | 1.56 |
| 774R | 1.19 |
| 775R | 1.09 |
| 776R | 1.19 |
| 779R | 1.55 |
| 780R | 1.18 |
| 781R | 1.66 |
| 782R | 1.24 |
| 783R | 0.06 |
| 784R | 1.51 |
| 785R | 1.77 |
| 786R | 1.54 |
| 787R | 0.06 |
| 789R | 1.41 |
| 790R | 1.06 |
| 791R | 0.11 |
| 792R | 1.84 |
| 794R | 1.49 |
| 795R | 0.30 |
| 797R | 1.08 |
| 798R | 1.40 |
| 800R | 0.09 |
| 801R | 0.23 |
| 802R | 1.33 |
| 804R | 0.61 |
| 805R | 0.22 |
| 806R | 0.13 |
| 807R | 0.11 |
| 808R | 0.90 |
| 809R | 0.62 |
| 810R | 1.11 |
| 811R | 0.22 |
| 812R | 0.94 |
| 813R | 0.99 |
| 814R | 0.42 |
| 815R | 0.47 |
| 817R | 1.16 |
| 818R | 0.23 |
| 819R | 1.14 |
| 821R | 0.91 |
| 822R | 1.48 |
| 823R | 1.76 |
| 824R | 0.32 |
| 825R | 1.46 |
| 826R | 1.60 |
| 827R | 0.34 |
| 828R | 1.19 |
| 829R | 1.73 |
| 830R | 1.79 |
| 831R | 0.76 |
| 832R | 1.25 |
| 833R | 1.51 |
| 834R | 1.40 |
| 835R | 1.32 |
| 836R | 1.53 |
| 837R | 1.32 |
| 838R | 1.21 |
| 839R | 1.30 |
| 840R | 1.36 |
| 841R | 0.11 |
| 842R | 1.58 |
| 844R | 1.14 |
| 845R | 1.72 |
| 846R | 1.91 |
| 847R | 1.61 |
| 848R | 1.38 |
| 849R | 0.07 |
| 850R | 1.74 |
| 851R | 1.59 |
| 852R | 1.22 |
| 853R | 1.67 |
| 854R | 0.08 |
| 855R | 1.48 |
| 856R | 1.45 |
| 857R | 1.19 |
| 858R | 1.87 |
| 859R | 1.40 |
| 860R | 1.84 |
| 862R | 1.14 |
| 863R | 1.27 |
| 864R | 0.80 |
| 865R | 1.07 |
| 866R | 1.58 |
| 867R | 0.19 |
| 868R | 0.54 |
| 870R | 0.90 |
| 872R | 0.14 |
| 873R | 1.11 |
| 874R | 0.49 |
| 875R | 1.03 |
| 876R | 0.05 |
| 877R | 0.96 |
| 879R | 1.23 |
| 880R | 0.85 |
| 881R | 1.39 |
| 882R | 0.93 |
| 883R | 1.13 |
| 884R | 1.43 |
| 885R | 0.13 |
| 886R | 1.25 |
| 887R | 1.12 |
| 888R | 0.05 |
| 890R | 0.05 |
| 891R | 0.06 |
| 892R | 1.42 |
| 893R | 1.64 |
| 894R | 0.04 |
| 895R | 0.31 |
| 896R | 0.74 |
| 897R | 0.53 |
| 898R | 1.07 |
| 899R | 1.33 |
| 900R | 1.44 |
| 901R | 1.30 |
| 902R | 0.30 |
| 903R | 1.27 |
| 904R | 1.62 |
| 905R | 1.36 |
| 906R | 0.15 |
| 909R | 1.05 |
| 910R | 1.18 |
| 911R | 0.17 |
| 912R | 0.06 |
| 913R | 1.32 |
| 914R | 0.26 |
| 916R | 1.29 |
| 917R | 0.31 |
| 918R | 0.40 |
| 919R | 1.04 |
| 920R | 0.47 |
| 921R | 0.38 |
| 922R | 1.36 |
| 923R | 0.43 |
| 924R | 1.25 |
| 925R | 0.11 |
| 926R | 1.49 |
| 927R | 0.19 |
| 928R | 0.59 |
| 930R | 0.83 |
| 931R | 1.10 |
| 932R | 1.21 |
| 933R | 0.87 |
| 934R | 0.94 |
| 935R | 0.13 |
| 936R | 0.45 |
| 937R | 0.19 |
| 938R | 0.90 |
| 939R | 0.49 |
| 940R | 0.04 |
| 941R | 0.05 |
| 942R | 0.10 |
| 943R | 0.11 |
| 944R | 0.11 |
| 945R | 0.08 |
| 946R | 0.05 |
| 947R | 0.94 |
| 948R | 0.10 |
| 949R | 1.05 |
| 950R | 0.95 |
| 951R | 0.88 |
| 952R | 0.85 |
| 953R | 1.02 |
| 954R | 1.11 |
| 955R | 0.42 |
| 956R | 1.44 |
| 957R | 0.85 |
| 958R | 1.20 |
| 959R | 0.46 |
| 960R | 1.20 |
| 961R | 0.07 |
| 963R | 0.11 |
| 964R | 1.10 |
| 965R | 1.06 |
| 966R | 0.95 |
| 967R | 1.17 |
| 968R | 0.19 |
| 969R | 0.84 |
| 970R | 0.57 |
| 971R | 0.09 |
| 972R | 0.84 |
| 973R | 0.46 |
| 974R | 0.96 |
| 975R | 0.36 |
| 976R | 0.53 |
| 977R | 0.76 |
| 979R | 0.07 |
| 980R | 0.55 |
| 981R | 1.20 |
| 982R | 0.67 |
| 983R | 0.15 |
| 985R | 1.81 |
| 986R | 0.89 |
| 987R | 0.08 |
| 988R | 1.60 |
| 989R | 1.55 |
| 990R | 1.06 |
| 991R | 0.84 |
| 992R | 1.56 |
| 993R | 1.43 |
| 994R | 0.80 |
| 995R | 0.26 |
| 996R | 1.51 |
| 997R | 1.32 |
| 998R | 0.10 |
| 999R | 0.12 |
| 1000R | 0.08 |
| 1001R | 0.97 |
| 1002R | 0.07 |
| 1003R | 0.17 |
| 1004R | 0.13 |
| 1005R | 0.45 |
| 1006R | 0.09 |
| 1007R | 0.91 |
| 1008R | 0.13 |
| 1009R | 1.39 |
| 1010R | 0.67 |
| 1011R | 0.99 |
| 1012R | 1.06 |
| 1013R | 0.10 |
| 1014R | 1.04 |
| 1015R | 0.09 |
| 1016R | 1.42 |
| 1017R | 1.06 |
| 1018R | 0.77 |
| 1021R | 0.89 |
| 1023R | 1.00 |
| 1024R | 1.12 |
| 1025R | 0.86 |
| 1026R | 0.56 |
| 1027R | 0.98 |
| 1028R | 0.78 |
| 1029R | 1.00 |
| 1030R | 0.90 |
| 1031R | 0.11 |
| 1032R | 1.17 |
| 1033R | 1.40 |
| 1035R | 0.09 |
| 1036R | 1.33 |
| 1037R | 1.17 |
| 1038R | 1.31 |
| 1039R | 1.17 |
| 1040R | 1.10 |
| 1041R | 1.18 |
| 1042R | 0.06 |
| 1043R | 1.09 |
| 1044R | 1.29 |
| 1045R | 1.42 |
| 1046R | 0.11 |
| 1047R | 0.83 |
| 1048R | 0.05 |
| 1049R | 0.05 |
| 1050R | 1.50 |
| 1052R | 0.07 |
| 1053R | 0.05 |
| 1055R | 1.19 |
| 1056R | 0.12 |
| 1057R | 0.08 |
| 1058R | 0.06 |
| 1059R | 1.05 |
| 1060R | 0.84 |
| 1061R | 1.29 |
| 1062R | 0.11 |
| 1063R | 0.07 |
| 1064R | 0.69 |
| 1065R | 1.05 |
| 1066R | 0.43 |
| 1067R | 1.33 |
| 1068R | 1.11 |
| 1069R | 0.84 |
| 1070R | 1.07 |
| 1071R | 1.10 |
| 1072R | 1.10 |
| 1073R | 1.44 |
| 1074R | 1.50 |
| 1075R | 1.16 |
| 1076R | 0.62 |
| 1077R | 0.34 |
| 1078R | 1.05 |
| 1079R | 0.07 |
| 1080R | 0.23 |
| 1081R | 0.09 |
| 1082R | 0.08 |
| 1083R | 0.06 |
| 1084R | 0.06 |
| 1085R | 0.10 |
| 1086R | 0.06 |
| 1087R | 0.06 |
| 1088R | 0.05 |
| 1089R | 0.06 |
| 1090R | 0.03 |
| 1091R | 0.10 |
| 1092R | 0.20 |
| 1093R | 0.27 |
| 1094R | 0.23 |
| 1095R | 1.41 |
| 1096R | 1.16 |
| 1097R | 1.15 |
| 1098R | 1.15 |
| 1100R | 1.59 |
| 1101R | 1.38 |
| 1102R | 1.29 |
| 1103R | 1.32 |
| 1104R | 1.35 |
| 1105R | 1.34 |
| 1107R | 1.39 |
| 1108R | 1.22 |
| 1109R | 0.20 |
| 1110R | 0.44 |
| 1111R | 1.20 |
| 1112R | 1.20 |
| 1113R | 1.02 |
| 1115R | 1.21 |
| 1116R | 1.28 |
| 1117R | 1.06 |
| 1120R | 1.26 |
| 1122R | 1.21 |
| 1123R | 1.22 |
| 1124R | 1.40 |
| 1125R | 1.30 |
| 1128R | 0.78 |
| 1129R | 1.40 |
| 1130R | 1.05 |
| 1131R | 1.34 |
| 1132R | 1.92 |
| 1133R | 1.32 |
| 1134R | 1.29 |
| 1135R | 1.37 |

In the table, nR (n is an integer) denotes that the amino acid residue at position n is mutated to R.

Construction of clones with different mutation position combinations of Cas mutant proteins and verification of editing activity

According to the editing efficiency results of different mutation positions mentioned above, different mutation positions were selected for multiple mutation combinations, and the construction of plasmid of the Cas mutant with double-position 426 and 860 was introduced as an example.

Using C12-279-pCDH plasmid as the template, ChkCas12-PF1+279_426_R was subjected to PCR amplification to obtain the mutation fragment D426R-F1, 279_426_F+279_860_R was subjected to PCR amplification to obtain the mutation fragment D426R-L860R-F1, and ChkCas12-PR2+279_860_F was subjected to PCR amplification to obtain the mutation fragment L860R-F2. The plasmid C12-279-pCDH was digested with HindIII+KpnI, and the 5647 bp vector fragment was recovered by gel extraction, and was recombined in vitro with fragments D426R-F1, D426R-L860R-F1, and L860R-F2, and the mutant plasmid C12-279-pCDH-426-860 was obtained by heat shock transformation of Escherichia coli. The construction manner of other plasmids with multiple mutations was consistent with that of C12-279-pCDH-426-860.

The amino acid sequence of the Mut-02-1-426-846-858-860 mutation is:

(SEQ ID NO: 760)
RFVVLSVNQFRCNPNAFRQASMKTDDEKKDTAIKSYTSWLLPNDRKEKDM
VRSFLALDQGSRFFFDLMQAWYGGLTPDIIKSVTKERDLVDLWCAIYWFR
PMKKELASHPIDRVDLAKTFQRYYGGPASDVVQEYLSASIGEDCCWNDCR
QKYQEFCKNLGVDFTPDLKTLVREKLIAVALNDGSRAAISNLFGNGGKED
RNVKVNICKKILIALEKAGKSVKYVRDVQSIILKCANAKDREDFNRIYAD
KNGRPGTLLLLLDRKGENTYDAEVLKRYLRKTIKSKNAPLVWNYNQKLFE
YIERGIRNLLRRTINYDAWAWGEMWRVGHTPLQTKATRNYHFAQEQLKRH
SRIAAASQSSHAIAVALNEFFESPFFDGENPFTICPHHLGKNLEKLFKAW
ADEKNQTDEEDAGETAITDYCAEFKRGFDREPIRNLLRYVYASLRSKYSA
KELIQAAKYNQQFERYQRQKAHPVTPDNQGYTWPEAVIKPSKAERKNREN
SLDTRIWVSVKLLQKDGTWEKHHIPFYNLRFFEEVYAAASIAEDMPSPTF
RNSRFGFKLPKLLNTHAKINKKKKDGKKDKKAVNAAKREARIYLAAKEGR
LPAPSVPLDKLSAVIARVNGKFQVTVPVKFKVNKQPKYPLPQLGQIILGY
DQNLTANHAWALFEVVTEDTPGAHFCRNQYLRVLKTGQVRSITKTKDGQE
IDQLSYDGLEYKEYGEWRKQAKKFASQWYITTKVRKGKETTLLSQPALER
FESIEKYKPALYRENKEYARLLLAVIRRKTLNELEEIRPEIFRLVEQGFG
ICRFGSLSLSSFEAIRAAKAVIYGYFSTALKHDHNTPITDDERQARDPEL
FGLLNKLRTRRQNKGEEKINRAANALIRIALENKADFIRGEGDLPTTNKS
VKKGQNSRSMDWLARGLFNKICQLAVMHNIIPTAANPHSTSHQDPFEHNK
DFDDPQPAMKCRYAEFKIEDIGDSVLERLGTSLRNAKAGQTGAYYYKGAQ
DFLAHYGLQDIEEELKKCRRGRKANMPCWELQKRLIQKLGSKDAVVYIPM
RGGRIYLATHSVTTGAKPIIFNGEEVWVSNADEIAAVNIGLTIIPTSKKD
EEHPDRENDDKAIRKSVRRPRKSGERRSGDKMPATARKT.

The editing activity of multi-point mutations was verified using the same manner as described above in this example.

The results indicate that the editing efficiency of most multi-point mutants are improved compared with the wild-type, and specific results are shown in Table 21 and FIG. 19. The absolute value of the editing efficiency of the wild type C12-279 group is 8.7%.

TABLE 21
Editing efficiency of each mutant of C12-279 in Reporter3 cell
line (expressed as multiple of editing efficiency of C12-279)γ

| | Editing | | Editing |
|---|---|---|---|
| | efficiency | | efficiency |
| | (expressed | | (expressed |
| | as multiple | | as multiple |
| | of the | | of the |
| | editing | | editing |
| | efficiency | | efficiency |
| Group | of C12-279) | Group | of C12-279) |
| Wt wild type | 1.00 | 858 + 988 + 1132 | 1.97 |
| 3 | 1.71 | 988 + 1132 | 1.53 |
| 5 | 1.66 | Mut-01 | 1.58 |
| 7 | 1.48 | Mut-01 + 376 | 1.99 |
| 333 | 1.57 | Mut-01 + 485 | 1.59 |
| 376 | 1.59 | Mut-02 | 1.72 |
| 426 | 1.85 | Mut-02 + 1 + 3 + 426 + 858 + 860 | 3.70 |
| 485 | 1.48 | Mut-02 + 1 + 3 + 426 + 860 | 3.35 |
| 846 | 1.61 | Mut-02 + 1 + 333 + 426 + 858 + 860 | 3.68 |
| 858 | 1.57 | Mut-02 + 1 + 333 + 426 + 860 | 2.84 |
| 860 | 1.78 | Mut-02 + 1 + 426 + 485 + 858 + 860 | 3.67 |
| 988 | 1.66 | Mut-02 + 1 + 426 + 485 + 860 | 3.25 |
| 1132 | 1.70 | Mut-02 + 1 + 426 + 846 + 858 + 860 | 3.88 |
| 2 + 5 + 846 + 858 | 2.68 | Mut-02 + 1 + 426 + 846 + 860 | 3.83 |
| 3 + 1132 | 0.66 | Mut-02 + 1 + 5 + 426 + 858 + 860 | 3.77 |
| 3 + 333 + 426 | 2.29 | Mut-02 + 1 + 5 + 426 + 860 | 3.62 |
| 3 + 426 + 858 | 2.51 | Mut-02 + 1132 | 2.10 |
| 3 + 426 + 860 | 2.42 | Mut-02 + 3 | 0.13 |
| 3 + 5 | 2.06 | Mut-02 + 3 + 376 + 426 | 2.57 |
| 3 + 7 | 1.38 | Mut-02 + 3 + 426 | 2.66 |
| 3 + 846 | 1.71 | Mut-02 + 3 + 426 + 858 + 860 | 3.58 |
| 3 + 846 + 1132 | 1.92 | Mut-02 + 3 + 426 + 860 | 3.68 |
| 3 + 846 + 858 | 2.50 | Mut-02 + 333 | 2.03 |
| 3 + 846 + 860 | 2.53 | Mut-02 + 333 + 336 + 352 + 376 + 426 | 0.13 |
| 3 + 846 + 988 | 2.32 | Mut-02 + 333 + 352 + 376 + 426 | 2.24 |
| 3 + 858 | 2.15 | Mut-02 + 333 + 426 | 2.40 |
| 3 + 858 + 860 | 2.48 | Mut-02 + 333 + 426 + 858 + 860 | 3.54 |
| 3 + 858 + 988 | 2.18 | Mut-02 + 333 + 426 + 860 | 3.49 |
| 3 + 860 | 2.41 | Mut-02 + 376 | 0.14 |
| 333 + 376 | 1.94 | Mut-02 + 376 + 426 | 2.54 |
| 333 + 376 + 426 | 2.75 | Mut-02 + 376 + 426 + 485 + 860 | 2.21 |
| 333 + 376 + 485 | 1.83 | Mut-02 + 376 + 426 + 860 + 865 | 2.57 |
| 333 + 426 | 2.47 | Mut-02 + 426 | 2.36 |
| 333 + 426 + 1132 | 2.27 | Mut-02 + 426 + 1132 | 1.87 |
| 333 + 426 + 485 | 2.33 | Mut-02 + 426 + 485 | 2.28 |
| 333 + 426 + 846 | 2.34 | Mut-02 + 426 + 485 + 858 + 860 | 3.06 |
| 333 + 426 + 858 | 2.28 | Mut-02 + 426 + 485 + 860 | 2.04 |
| 333 + 426 + 860 | 1.77 | Mut-02 + 426 + 485 + 860 | 3.04 |
| 333 + 485 | 1.79 | Mut-02 + 426 + 846 | 2.65 |
| 352 + 426 | 1.79 | Mut-02 + 426 + 846 + 858 + 860 | 3.29 |
| 376 + 426 | 1.94 | Mut-02 + 426 + 846 + 860 | 3.48 |
| 376 + 426 + 485 + 660 | 1.86 | Mut-02 + 426 + 860 | 3.06 |
| 376 + 485 | 1.51 | Mut-02 + 485 | 1.31 |
| 426 + 649 | 2.05 | Mut-02 + 5 | 2.69 |
| 426 + 846 + 858 + 860 | 3.02 | Mut-02 + 5 + 426 | 2.77 |
| 426 + 858 + 860 | 3.15 | Mut-02 + 5 + 426 + 858 + 860 | 3.58 |
| 426 + 858 + 9880 | 0.62 | Mut-02 + 5 + 426 + 860 | 3.38 |
| 428 + 485 | 2.26 | Mut-02 + 639 | 2.18 |
| 5 + 1132 | 1.45 | Mut-02 + 7 | 2.60 |
| 5 + 333 + 4260 | 2.97 | Mut-02 + 858 | 2.48 |
| 5 + 426 + 858 | 2.87 | Mut-02 + 860 | 3.00 |
| 5 + 426 + 860 | 3.30 | Mut-02 + 988 | 1.75 |
| 5 + 846 | 1.84 | Mut-03 | 2.01 |
| 5 + 846 + 1132 | 1.90 | Mut-03 + 1132 | 1.61 |
| 5 + 846 + 858 | 2.65 | Mut-03 + 3 | 1.94 |
| 5 + 846 + 860 | 2.32 | Mut-03 + 3 + 107 + 426 | 2.81 |
| 5 + 858 | 2.15 | Mut-03 + 3 + 376 | 2.44 |
| 5 + 858 + 988 | 2.18 | Mut-03 + 3 + 639 | 2.40 |
| 5 + 860 | 1.73 | Mut-03 + 333 | 1.78 |
| 7 + 426 + 846 | 3.02 | Mut-03 + 333 + 336 | 0.87 |
| 7 + 426 + 858 | 3.09 | Mut-03 + 376 | 2.30 |
| 846 + 1132 | 1.66 | Mut-03 + 376 + 1132 | 2.99 |
| 846 + 585 + 860 | 2.36 | Mut-03 + 426 | 2.20 |
| 846 + 858 | 1.96 | Mut- 03 + 426 + 1132 | 2.11 |
| 846 + 858 + 1132 | 1.77 | Mut-03 + 485 | 1.51 |
| 846 + 858 + 988 | 2.51 | Mut-03 + 5 | 2.08 |
| 846 + 860 | 1.99 | Mut-03 + 639 | 2.13 |
| 846 + 860 + 1132 | 1.84 | Mut-03 + 639 + 1132 | 1.82 |
| 846 + 860 + 988 | 2.42 | Mut-03 + 7 | 1.19 |
| 846 + 988 | 1.72 | Mut-03 + 846 | 2.13 |
| 858 + 860 | 2.23 | Mut-03 + 858 | 2.06 |
| 858 + 860 + 1132 | 2.06 | Mut-03 + 860 | 2.47 |
| 858 + 988 | 2.11 | | |

In the table, the integer n indicates that the amino acid residue at position n is mutated to R. Mut-01 is a Q186R mutant, Mut-02 is a Q186R & D352R double-point mutant, and Mut-03 is a G184R+Q186R+D352R triple-point mutant; 5+426+860 indicates that the amino acid residues at positions 5, 426, and 860 are mutated to R, simultaneously; Mut-02+860 indicates a multi-point mutant obtained by introducing an additional mutation at position 860 (mutated to R) based on the Mut-02 mutant; and other multi-point mutants are similar (mutated to R).

### Example 13. Detection of Editing Efficiency of Different Mutants for Different Target Sites of TTR and HBG

In this example, for the TTR gene (GenBank® database accession number: NG_009490.1) and the HBG gene (GenBank® database accession number: NC_000011.10), the PAM sequence was selected as the target site of TTN, and sgRNAs targeting different positions were designed and constructed (as shown in Table 22) for combination with different mutants to detect the editing efficiency.

TABLE 22
sgRNA targeting TTR and HBG

| sgRNA | | | |
|---|---|---|---|
| name | PAM | Target site | sgRNA sequence |
| C12-279- | TTC | catgagcatgcagaggtgagt | gtaatgcgtctcccattgacgccatgagcatgcagaggtg |
| TTR01 | | at (SEQ ID NO: 722) | agtat (SEQ ID NO: 783) |
| C12-279- | TTT | tcctataaggtgtgaaagtctg | gtaatgcgtctcccattgacgctcctataaggtgtgaaagt |
| TTR02 | | g (SEQ ID NO: 761) | ctgg (SEQ ID NO: 784) |
| C12-279- | TTC | cagtaagatttggtgtctat | gtaatgcgtctcccattgacgccagtaagatttggtgtctat |
| TTR03 | | (SEQ ID NO: 762) | (SEQ ID NO: 785) |
| C12-279- | TTG | tatatcccttctacaaattcctc | gtaatgcgtctcccattgacgctatatcccttctacaaattcc |
| TTR04 | | (SEQ ID NO: 763) | tc (SEQ ID NO: 786) |
| C12-279- | TTT | GTGTCTGAGGCTG | gtaatgcgtctcccattgacgcGTGTCTGAGGC |
| TTR05 | | GCCCTACGGTG | TGGCCCTACGGTG (SEQ ID NO: 787) |
| | | (SEQ ID NO: 764) | |
| C12-279- | TTC | AGAAAGGCTGCTG | gtaatgcgtctcccattgacgcAGAAAGGCTGC |
| TTR06 | | ATGACACCTG | TGATGACACCTG (SEQ ID NO: 788) |
| | | (SEQ ID NO: 765) | |
| C12-279- | TTG | GCATCTCCCCATTC | gtaatgcgtctcccattgacgcGCATCTCCCCAT |
| TTR07 | | CATGAGCATG | TCCATGAGCATG (SEQ ID NO: 789) |
| | | (SEQ ID NO: 766) | |
| C12-279- | TTC | ACAGCCAACGACT | gtaatgcgtctcccattgacgcACAGCCAACGA |
| TTR08 | | CCGGCCCCG (SEQ | CTCCGGCCCCCG (SEQ ID NO: 790) |
| | | ID NO: 767) | |
| C12-279- | TTG | TAGAAGGGATATA | gtaatgcgtctcccattgacgcTAGAAGGGATA |
| TTR09 | | CAAAGTGGAA | TACAAAGTGGAA (SEQ ID NO: 791) |
| | | (SEQ ID NO: 768) | |
| C12-279- | TTC | TAGATGCTGTCCG | gtaatgcgtctcccattgacgcTAGATGCTGTC |
| TTR10 | | AG GCAGTCCTG | CGAGGCAGTCCTG (SEQ ID NO: 792) |
| | | (SEQ ID NO: 769) | |
| C12-279- | TTT | GGTGTCTATTTCCA | gtaatgcgtctcccattgacgcGGTGTCTATTTC |
| TTR11 | | CTTTGTATA (SEQ | CACTTTGTATA (SEQ ID NO: 793) |
| | | ID NO: 770) | |
| C12-279- | TTT | CTGAACACATGCA | gtaatgcgtctcccattgacgcCTGAACACATG |
| TTR12 | | CGGCCACATTG | CACGGCCACATTG (SEQ ID NO: 794) |
| | | (SEQ ID NO: 771) | |
| C12-279- | TTA | CCCAGAGGCAAAT | gtaatgcgtctcccattgacgcCCCAGAGGCAA |
| TTR13 | | GGCTCCCAGG | ATGGCTCCCAGG (SEQ ID NO: 795) |
| | | (SEQ ID NO: 772) | |
| C12-279- | TTC | CTCCTCAGTTGTG | gtaatgcgtctcccattgacgcCTCCTCAGTIGT |
| TTR14 | | AGCCCATGCA | GAGCCCATGCA (SEQ ID NO: 796) |
| | | (SEQ ID NO: 773) | |
| C12-279- | TTG | GCCAGCCTTGCCT | gtaatgcgtctcccattgacgcGCCAGCCTTGC |
| HBG01 | | TGACCAATAG | CTTGACCAATAG (SEQ ID NO: 797) |
| | | (SEQ ID NO: 774) | |
| C12-279- | TTG | ACCAATAGTCTTA | gtaatgcgtctcccattgacgcACCAATAGTCT |
| HBG02 | | GAGTATCCAG | TAGAGTATCCAG (SEQ ID NO: 798) |
| | | (SEQ ID NO: 775) | |
| C12-279- | TTG | CCTTGTCAAGGCT | gtaatgcgtctcccattgacgcCCTTGTCAAGG |
| HBG03 | | ATTGGTCAAG | CTATTGGTCAAG (SEQ ID NO: 799) |
| | | (SEQ ID NO: 776) | |
| C12-279- | TTG | CCTTGACCAATAG | gtaatgcgtctcccattgacgcCCTTGACCAAT |
| HBG04 | | CCTTGACAAG | AGCCTTGACAAG (SEQ ID NO: 800) |
| | | (SEQ ID NO: 777) | |
| C12-279- | TTG | ACCAATAGCCTTG | gtaatgcgtctcccattgacgcACCAATAGCCT |
| HBG05 | | ACAAGGCAAA | TGACAAGGCAAA (SEQ ID NO: 801) |
| | | (SEQ ID NO: 778) | |
| C12-279- | TTG | GTCAAGGCAAGGC | gtaatgcgtctcccattgacgcGTCAAGGCAAG |
| HBG06 | | TGGCCAACCC | GCTGGCCAACCC (SEQ ID NO: 802) |
| | | (SEQ ID NO: 779) | |
| C12-279- | TTG | GTCAAGTTTGCCT | gtaatgcgtctcccattgacgcGTCAAGTTTTGC |
| HBG07 | | GTCAAGGC (SEQ | CTTGTCAAGGC (SEQ ID NO: 803) |
| | | ID NO: 780) | |
| C12-279- | TTG | TCAAGGCTATTGG | gtaatgcgtctcccattgacgcTCAAGGCTATT |
| HBG08 | | TCAAGGCAAG | GGTCAAGGCAAG (SEQ ID NO: 804) |
| | | (SEQ ID NO: 781) | |
| C12-279- | TTG | ACAAGGCAAACTT | gtaatgcgtctcccattgacgcACAAGGCAAAC |
| HBG09 | | GACCAATAGTC | TTGACCAATAGTC (SEQ ID NO: 805) |
| | | (SEQ ID NO: 782) | |

The sgRNA plasmids were constructed according to the sgRNA sequences in the above table: the vector plasmid SpCas9-gRNA-pUC57Kan was linearized using BbsI (Thermo Fisher Scientific™) and XhoI (Thermo Fisher Scientific™), the primers were synthesized for different sgRNA sequences, annealed and connected to the linearized vector, and Escherichia coli was transformed to obtain the final sgRNA expression vector plasmid.

Different mutant plasmids were combined with different sgRNA plasmids, and HEK293T cells were transfected with PEI. After 48 h, the cells were collected and lysed using DirectPCR® Lysis Reagent (cell lysis reagent) (Viagen™: 302-C). Different primers were selected according to different target sites for PCR amplification and Sanger sequencing. The editing efficiency was analyzed using TIDE.

Specific amplification and sequencing primers were shown in Table 23. The experiment was repeated for three times, and the editing efficiency results are shown in FIGS. 20A and 20B. Each sgRNA group in each batch of experiments generates effective editing, and the editing efficiency at many target sites is better than that of wild-type C12-297.

TABLE 23
Primers for amplification and sequencing of different target sites

| Amplification | | Corresponding | Sequencing |
|---|---|---|---|
| primer name | Primer sequence | sgRNA | primer |
| ChkTTR-PF1 | GAACGAATGTTCCGATGCTCTAAT C | C12-279-TTR05 | ChkTTR-PF1 |
| | (SEQ ID NO: 806) | | |
| ChkTTR-PR1 | AATGAGGTAGAATGCTCAGAGTTCA | | |
| | AGT (SEQ ID NO: 807) | | |
| ChkTTR-PF2 | CAACTGTTCTCAGGGGGACCTATTTT | C12-279-TTR06, | ChkTTR-PF2 |
| | C (SEQ ID NO: 808) | C12-279-TTR10, | |
| ChkTTR-PR2 | CTCTACTGTCTGCCCCTAAATGATGC | C12-279-TTR12, | |
| | (SEQ ID NO: 809) | C12-279-TTR13 | |
| ChkTTR-PF3 | TGCCATTTAATTTCAGTTAGGAGTTTT | C12-279-TTR01, | ChkTTR-PF3 |
| | C (SEQ ID NO: 810) | C12-279-TTR02, | |
| | | C12-279-TTR03, | |
| | | C12-279-TTR04, | |
| ChkTTR-PR3 | CAGACCTCCGAGAAGCCAGATGT | C12-279-TTR07, | |
| | (SEQ ID NO: 811) | C12-279-TTR09, | |
| | | C12-279-TTR11 | |
| | | C12-279-TTR14 | |
| ChkTTR-PF4 | CAAGACCTGACTCTGTACTCCTGCTC | C12-279-TTR08 | ChkTTR-PR4 |
| | T (SEQ ID NO: 812) | | |
| ChkTTR-PR4 | ATTTGGATCTCTCCTAGCGTTCTGC | | |
| | (SEQ ID NO: 813) | | |
| ChkHBG01-PF1 | ACGGCTGACAAAAGAAGTCCTGG | C12-279-HBG01 | ChkHBG01-PR1 |
| | (SEQ ID NO: 814) | to | |
| ChkHBG01-PR1 | CATGGGTAGACAACCAGGAGCC | C12-279-HBG09 | |
| | (SEQ ID NO: 815) | | |

### Example 14. Preparation and Activity Detection of Inactivated C12-279 Mutant

On the basis of the multi-point mutants designed in the previous examples, any one or more of the D651A, E891A, and D1082A mutations were further introduced, and the mutation clone plasmid was constructed using the same manner as in the previous example 10. Editing was performed in the Reporter3 cell line, and an analysis was performed on the proportion of cells that restored GFP expression after editing by flow cytometry to determine the editing efficiency of the mutant. Specific results are shown in FIG. 21. As long as each mutation in the figure contains at least one of D651A, E891A, and D1082A mutations, the editing efficiency is reduced to less than 1.3%. DeadCas12 (dCas12) may be obtained by introducing any of D651A, E891A, and D1082A.

The amino acid sequence of the dCas mutant Mut-02-1-426-846-858-860-D651A-E891A-D1082A is:

(SEQ ID NO: 816)
RFVVLSVNQFRCNPNAFRQASMKTDDEKKDTAIKSYTSWLLPNDRKEKDM
VRSFLALDQGSRFFFDLMQAWYGGLTPDIIKSVTKERDLVDLWCAIYWFR
PMKKELASHPIDRVDLAKTFQRYYGGPASDVVQEYLSASIGEDCCWNDCR
QKYQEFCKNLGVDFTPDLKTLVREKLIAVALNDGSRAAISNLFGNGGKED
RNVKVNICKKILIALEKAGKSVKYVRDVQSIILKCANAKDREDFNRIYAD
KNGRPGTLLLLLDRKGENTYDAEVLKRYLRKTIKSKNAPLVWNYNQKLFE
YIERGIRNLLRRTINYDAWAWGEMWRVGHTPLQTKATRNYHFAQEQLKRH
SRIAAASQSSHAIAVALNEFFESPFFDGENPFTICPHHLGKNLEKLFKAW
ADEKNQTDEEDAGETAITDYCAEFKRGFDREPIRNLLRYVYASLRSKYSA
KELIQAAKYNQQFERYQRQKAHPVTPDNQGYTWPEAVIKPSKAERKNREN
SLDTRIWVSVKLLQKDGTWEKHHIPFYNLRFFEEVYAAASIAEDMPSPTF
RNSRFGFKLPKLLNTHAKINKKKKDGKKDKKAVNAAKREARIYLAAKEGR
LPAPSVPLDKLSAVIARVNGKFQVTVPVKFKVNKQPKYPLPQLGQIILGY
AQNLTANHAWALFEVVTEDTPGAHFCRNQYLRVLKTGQVRSITKTKDGQE
IDQLSYDGLEYKEYGEWRKQAKKFASQWYITTKVRKGKETTLLSQPALER
FESIEKYKPALYRFNKEYARLLLAVIRRKTLNELEEIRPEIFRLVEQGFG
ICRFGSLSLSSFEAIRAAKAVIYGYFSTALKHDHNTPITDDERQARDPEL
FGLLNKLRTRRQNKGEEKINRAANALIRIALENKADFIRGAGDLPTTNKS
VKKGQNSRSMDWLARGLFNKICQLAVMHNIIPTAANPHSTSHQDPFEHNK
DFDDPQPAMKCRYAEFKIEDIGDSVLERLGTSLRNAKAGQTGAYYYKGAQ
DFLAHYGLQDIEEELKKCRRGRKANMPCWELQKRLIQKLGSKDAVVYIPM
RGGRIYLATHSVTTGAKPIIFNGEEVWVSNAAEIAAVNIGLTIIPTSKKD
EEHPDRENDDKAIRKSVRRPRKSGERRSGDKMPATARKT.

### Example 15. mRNA+gRNA Delivery

The TTR gene was edited using the encoding mRNA of the Mut-02-3-426-860 mutant (obtained by in vitro transcription) together with the modified gRNA (as shown in Table 24).

TABLE 24
Modified gRNA

| | | | Editing |
|---|---|---|---|
| | | | efficiency |
| | | | determined by |
| | gRNA sequence (including | | Sanger |
| gRNA name | modifications) | Modification | sequencing |
| C279-dmTTR01-01 | dAdTdGdTdGdTdTdTdTdTdGdT | adding a 25-nt DNA | 52.0% |
| | dCdAdAdAdAdGdAdCdCdTdTd | sequence to the 5′ | |
| | TdTrGrUrArArUrGrCrGrUrCrUr | end and modifying | |
| | CrCrArUrUrGrArCrGrCrCrArUr | the last nucleotide | |
| | GrArGrCrArUrGrCrArGrArGrGr | at the 3′ end with | |
| | UrGrArGrUrA*mU (SEQ ID NO: | phosphorothioate | |
| | 817) | and | |
| | | 2′-oxymethylation. | |
| C279-mTTR01-01 | mG*mU*mA*rArUrGrCrGrUrCr | modifying three | 29.5% |
| | UrCrCrArUrUrGrArCrGrCrArUr | nucleotides at the 5′ | |
| | GrArGrCrArUrGrCrArGrArGrGr | and 3′ ends with | |
| | UrGrArG*mU*mA*mU (SEQ ID | phosphorothioate | |
| | NO: 818) | and 2′-oxymethyl | |
| C279-dmTTR01-02 | dGdTdTdGdCdAdAdTdCdCdCd | adding a 14-nt DNA | 47.5% |
| | AdAdGrGrUrArArUrGrCrGrUrCr | sequence to the 5′ | |
| | UrCrCrArUrGrArCrGrCrCrArUr | end and modifying | |
| | GrArGrCrArUrGrCrArGrArGrGr | the last nucleotide | |
| | UrGrArGrUrA*mU (SEQ ID NO: | at the 3′ end with | |
| | 819) | phosphorothioate | |
| | | and 2′-oxymethyl. | |
| C279-mTTR01-02 | mG*rUrArArUrGrCrGrUrCrUrCr | modifying a base at | 26.8% |
| | CrArUrUrGrArCrGrCrArUrGrAr | the 5′ and 3′ ends | |
| | GrCrArUrGrCrArGrArGrGrUrGr | with | |
| | ArGrUrA*mU (SEQ ID NO: 820) | phosphorothioate | |
| | | and 2′-oxymethyl | |

HEK293 cells were transformed with mRNA and gRNA by electroporation. After 48 h, cells were collected and lysed using DirectPCR® Lysis Reagent (cell lysis reagent) (Viagen™: 302-C). After that, a Sanger sequencing was performed on the lysed cells, and the editing efficiency was analyzed using TIDE. The results are shown in Table 24.

After adding DNA nucleotide sequence at the 3′ end, the editing activity is significantly improved. For example, the added sequence is as follows.

(SEQ ID NO: 821)
dAdTdGdTdGdTdTdTdTdTdGdTdCdAdAdAdAdGdAdCdCdTdTdT
dT
or
(SEQ ID NO: 822)
dGdTdTdGdCdAdAdTdCdCdCdAdAdG.

In addition, primers TTR-NGS-PF2 (GCGTAACTTAATCCAGACTTTCACACCTT, SEQ ID NO: 823) and TTR-NGS-PR2 (GGTCATTCATCACCTTCCTTAGGACA, SEQ ID NO: 824) were used for PCR amplification and library construction, and an NGS sequencing was performed to detect the editing efficiency. The electroporation editing efficiency of a combination of the mutants Mut-02-3-426-860 and C279-dmTTR01-01 reaches 79.64%.

Subsequently, C279-dmTTR01-02 was tested in combination with different mutants. The specific NGS sequencing results are shown in Table 25 and FIG. 22.

TABLE 25
Editing efficiency of modified gRNA (C279-dmTTR01-
02) combined with different mutants (NGS assay)

| | | Editing efficiency |
|---|---|---|
| Mutant | sgRNA | determined by NGS |
| Mut-02-426-860 | C279-dmTTR01-02 | 24.63% |
| Mut-02-5-426-860 | C279-dmTTR01-02 | 63.48% |
| Mut-02-1-5-426-858-860 | C279-dmTTR01-02 | 92.18% |
| Mut-02-1-426-846-858-860 | C279-dmTTR01-02 | 88.05% |
| Mut-02-1-3-426-858-860 | C279-dmTTR01-02 | 89.67% |
| Mut-02-3-426-860 | C279-dmTTR01-02 | 79.99% |

### Example 16. Verification of Editing Efficiency of Different Endogenous Target Genes

Different gRNA target sites were designed according to different disease-related target sites (Table 26), and the mutant Mut-02-1-426-846-858-860 protein was edited in combination with the gRNA in Table 26. The same manner as in example 13 was used, including the plasmid construction, the transfection, and the editing efficiency detection. The results are shown in Table 26.

TABLE 26
Editing efficiency of targeting different endogenous genes

| | Target | Sequence of | | Editing |
|---|---|---|---|---|
| Name | gene | PAM position | gRNA guide sequence | efficiency |
| sgRNA-BCL11A-01-CT | BCL11A | TTC | ctggagcctgtgataaaagcaac | 27.40% |
| X-001-PAM-TTC | | | (SEQ ID NO: 825) | |
| sgRNA-BCL11A-02-CT | BCL11A | TGC | aagctaacagttgcttttatcac | 1% |
| X-001-PAM-TGC | | | (SEQ ID NO: 826) | |
| sgRNA-HBG-01-ZBTB7 | HBG | TTG | cattgagatagtgtggggaaggg | 10.40% |
| A-binding-site-PAM-TTG | | | (SEQ ID NO: 827) | |
| sgRNA-HBG-02-ZBTB7 | HBG | TTT | gcattgagatagtgtggggaagg | 10.90% |
| A-binding-site-PAM-TTT | | | (SEQ ID NO: 828) | |
| sgRNA-HBG-03-ZBTB7 | HBG | TTG | agatagtgtggggaaggggcccc | 23.20% |
| A-binding-site-PAM-TTG | | | (SEQ ID NO: 829) | |
| sgRNA-HBG-04-ZBTB7 | HBG | TTG | ggggccccttccccacactatct | 18.80% |
| A-binding-site-PAM-TTG | | | (SEQ ID NO: 830) | |
| sgRNA-HBG-05-ZBTB7 | HBG | TAT | cctcttgggggccccttccccac | 1% |
| A-binding-site-PAM-TAT | | | (SEQ ID NO: 831) | |
| sgRNA-HBG-06-ZBTB7 | HBG | GTA | tcctcttgggggccccttcccca | 1.20% |
| A-binding-site-PAM-GTA | | | (SEQ ID NO: 832) | |
| sgRNA-HBG-07-ZBTB7 | HBG | TTA | gcagtatcctcttgggggcccct | 1.60% |
| A-binding-site-PAM-TTA | | | (SEQ ID NO: 833) | |
| sgRNA-HBG-08-(-175)- | HBG | TTC | cccacactatctcaatgcaaata | 43.10% |
| PAM-TTC | | | (SEQ ID NO: 834) | |
| sgRNA-HBG-09-(-175)- | HBG | TTT | cagacagatatttgcattgagat | 39.40% |
| PAM-TTT | | | (SEQ ID NO: 835) | |
| sgRNA-HBG-10-(-175)- | HBG | TTT | agccagggaccgtttcagacaga | 34.20% |
| PAM-TTT | | | (SEQ ID NO: 836) | |
| sgRNA-HBG-11-(-88)- | HBG | TTG | acaaggcaaacttgaccaatagt | 33.20% |
| PAM-TTG | | | (SEQ ID NO: 837) | |
| sgRNA-HBG-12-(-73)- | HBG | TTG | accaatagtcttagagtatccag | 14.80% |
| PAM-TTG | | | (SEQ ID NO: 775) | |
| sgRNA-HBG-13-(-56)- | HBG | TTC | atccctagccagccgccggcccc | 14.70% |
| PAM-TTC | | | (SEQ ID NO: 838) | |
| sgRNA-BACH2-01-PAM- | BACH2 | TTG | tagggtaccctcgtgcacccggg | 9.60% |
| TTG | | | (SEQ ID NO: 839) | |
| sgRNA-BACH2-02-PAM- | BACH2 | TTC | cgcggcagccactcgctggtatc | 29.70% |
| TTC | | | (SEQ ID NO: 840) | |
| sgRNA-BACH2-03-PAM- | BACH2 | TTG | gtccttgaggacagacaccactg | 7.10% |
| TTG | | | (SEQ ID NO: 841) | |
| sgRNA-BACH2-04-PAM- | BACH2 | TTC | atccttgttttccagtggtgtct | 17% |
| TTC | | | (SEQ ID NO: 842) | |
| sgRNA-BACH2-05-PAM- | BACH2 | TTG | gaagagttgaccatcagttatgg | 28.70% |
| TTG | | | (SEQ ID NO: 843) | |
| KLKB1-sgRNA01 | KLKB1 | TTC | CTCTACTCCTCAAGAA | 19% |
| | | | ACACCA (SEQ ID NO: | |
| | | | 844) | |
| KLKB1-sgRNA02 | KLKB1 | TTG | ACACAGAACCCTGCC | 27.20% |
| | | | ATTCTAAA (SEQ ID NO: | |
| | | | 845) | |
| KLKB1-sgRNA03 | KLKB1 | TTC | AGTCTGCACAACAAA | 24.30% |
| | | | AACAAGCA (SEQ ID | |
| | | | NO: 846) | |
| KLKB1-sgRNA04 | KLKB1 | TTG | GGGTGATAGGTGCAGA | 21.20% |
| | | | TGGTCCG (SEQ ID NO: | |
| | | | 847) | |
| KLKB1-sgRNA05 | KLKB1 | TTA | CCCAACAGTTGGTATA | Not detected |
| | | | AATTGTG (SEQ ID NO: | |
| | | | 848) | |
| KLKB1-sgRNA06 | KLKB1 | TTT | CAGTTCTGCACAACA | 25.50% |
| | | | AAAACAAGC (SEQ ID | |
| | | | NO: 849) | |
| KLKB1-sgRNA07 | KLKB1 | TTC | AGGTGGTCCCTTAGTT | 14.60% |
| | | | TGCAAAC (SEQ ID NO: | |
| | | | 850) | |
| KLKB1-sgRNA08 | KLKB1 | TTT | GGGGTGTACATGGAA | 23.40% |
| | | | GCTACATC (SEQ ID | |
| | | | NO: 851) | |
| KLKB1-sgRNA09 | KLKB1 | TTG | CCTATTAAAGTACAGT | 1% |
| | | | CC CGGAG (SEQ ID NO: | |
| | | | 852) | |
| KLKB1-sgRNA10 | KLKB1 | TTA | TAGCGGTAGGGTTCCT | 9.40% |
| | | | CCGGGA (SEQ ID NO: | |
| | | | 853) | |
| PCSK9-sgRNA01 | PCSK9 | TTC | CGTGCTCGGGTGCTTC | 10% |
| | | | GGCCAGG (SEQ ID NO: | |
| | | | 854) | |
| PCSK9-sgRNA02 | PCSK9 | TTA | CCCCTCCACGGTACCG | 20.40% |
| | | | GGCGGAT (SEQ ID NO: | |
| | | | 855) | |
| PCSK9-sgRNA03 | PCSK9 | TTC | ATCCGCCCGGTACCGT | 17.60% |
| | | | GGAGGGG (SEQ ID NO: | |
| | | | 856) | |
| PCSK9-sgRNA04 | PCSK9 | TTG | GCAGTTGAGCACGCG | 21% |
| | | | CAGGCTGC (SEQ ID | |
| | | | NO: 857) | |
| PCSK9-sgRNA05 | PCSK9 | TTG | GCCACGCCGGCATCCC | 4.90% |
| | | | GCCCGCT (SEQ ID NO: | |
| | | | 858) | |
| PCSK9-sgRNA06 | PCSK9 | TTG | AGGACGCGGCTGTAC | 8.20% |
| | | | CCACCCGC (SEQ ID | |
| | | | NO: 859) | |
| PCSK9-sgRNA07 | PCSK9 | TTT | GGCCGCTGTGTGGAC | 7.80% |
| | | | CTCTTTGC (SEQ ID | |
| | | | NO: 860) | |
| PCSK9-sgRNA08 | PCSK9 | TTG | TCTACGGCGTAGGCCC | 29.00% |
| | | | CCAGGAC (SEQ ID NO: | |
| | | | 861) | |
| SOD1-sgRNA01 | SOD1 | TTG | CCCAAGTCTCCAACAT | 35.60% |
| | | | GCCTAAT (SEQ ID NO: | |
| | | | 862) | |
| SOD1-sgRNA02 | SOD1 | TTC | CCCACAACCTTCACTG | 32.10% |
| | | | GTCCATTA (SEQ ID NO: | |
| | | | 863) | |
| SOD1-sgRNA03 | SOD1 | TTA | ATGCTTCCCCACACCT | 34.30% |
| | | | TCACTGG (SEQ ID NO: | |
| | | | 864) | |
| SOD1-sgRNA04 | SOD1 | TTA | AGCATCTTGTTACCTC | Not detected |
| | | | TCTTCAT (SEQ ID NO: | |
| | | | 865) | |
| SOD1-sgRNA05 | SOD1 | TTG | GGCAATGTGACTGCTG | 38.20% |
| | | | ACAAAGA (SEQ ID NO: | |
| | | | 866) | |
| SOD1-sgRNA06 | SOD1 | TTC | AGCACGCACACGGCC | 21.50% |
| | | | TTCGTCGC (SEQ ID | |
| | | | NO: 867) | |
| SOD1-sgRNA07 | SOD1 | TTA | AAGGACTGACTGAAG | 33.20% |
| | | | GCCTGCAT (SEQ ID | |
| | | | NO: 868) | |
| BACE1-sgRNA01 | BACE1 | TTG | AGGGGGAAGCCAGCA | 28.90% |
| | | | CCACAAAG (SEQ ID | |
| | | | NO: 869) | |
| BACE1-sgRNA02 | BACE1 | TTC | CCTCTTAGTACAACTA | Not detected |
| | | | TGACAAG (SEQ ID NO: | |
| | | | 870) | |
| BACE1-sgRNA03 | BACE1 | TTC | CCAGTCATCTCACTCT | 24.50% |
| | | | ACCTAAT (SEQ ID NO: | |
| | | | 871) | |
| BACE1-sgRNA04 | BACE1 | TTG | CCCTCTCCCCAGTGCA | 18.90% |
| | | | CGATGAG (SEQ ID NO: | |
| | | | 872) | |
| BACE1-sgRNA05 | BACE1 | TTC | AGCAGGGAGATGTCAT | 27.90% |
| | | | CAGCAAA (SEQ ID NO: | |
| | | | 873) | |
| BACE1-sgRNA06 | BACE1 | TTA | GGTAGAGTGAGATGA | 10.80% |
| | | | CTGGGAAA (SEQ ID | |
| | | | NO: 874) | |
| BACE1-sgRNA07 | BACE1 | TTC | CCCCTCAACCAGTCTG | 35.30% |
| | | | AAGTGCT (SEQ ID NO: | |
| | | | 875) | |
| BACE1-sgRNA08 | BACE1 | TTC | AGATCCTGTCCATTGA | 5.70% |
| | | | TCTCCAC (SEQ ID NO: | |
| | | | 876) | |
| BACE1-sgRNA09 | BACE1 | TTG | GCACGCACAGTGACG | 4.30% |
| | | | TTGGGGCC (SEQ ID | |
| | | | NO: 877) | |

### Example 17. PAM Recognition of C12-279 Mutant Mut-02-1-426-846-858-860

Using the same manner as in the previous examples, the PAM recognized by the mutant Mut-02-1-426-846-858-860 was 5′-WTN-3′ (W is A or T) by performing bacterial editing experiments in vivo and NGS sequencing, as shown in FIG. 23.

Although the specific examples of the present disclosure are described above, those skilled in the art should understand that these are only examples, and that various changes or modifications may be made to these examples without departing from the principles and essence of the present disclosure. Therefore, the scope of protection of the present disclosure is limited by the attached claims.

### Example 18. Verification of Base Editing Activity

Based on the Mut-02-1-3-426-846-858-860 mutant, D651A, E891A and D1082A mutations were introduced to obtain dCas12 (SEQ ID NO: 975), a mutant version that lost the cutting ability. The deaminase domain was then fused to obtain a base editor. The editor fused with adenine deaminase was named ABE-dCas279-pCDNA3.1-01 (SEQ ID NO: 976). The base editing efficiency was tested for different targets.

The experimental methods such as sgRNA construction, transfection, and editing efficiency detection are basically the same as those in Example 13. The final results are shown in Table 28.

TABLE 28
Single base editing efficiency

| Target | | | Spacer | Base editing |
|---|---|---|---|---|
| Gene | PAM | Spacer Sequence | (SEQ ID NO) | efficiency |
| TTR | TTC | CATGAGCATGCAGAGGTGAGTAT | 722 | A8 base 14% |
| | | | | A12 base 11% |
| | | | | A22 base 10% |
| TTR | TTG | GCATCTCCCCATTCCATGAGCATG | 766 | A11 base 23% |
| TTR | TTG | TAGAAGGGATATACAAAGTGGAA | 768 | All base 23% |
| | | | | A13 base 9% |
| HBG | TTG | CCTTGACCAATAGCCTTGACAAG | 777 | A12 base 2% |

### Example 19. CrRNA Backbone Sequence Modification

The crRNA (guide sequence SEQ ID NO: 735) with mutation of DR sequence (skeleton sequence) was tested in combination with Mut-02-1-426-846-858-860 (named C12-279-V4). The editing system was transfected into the Reporter 3 cell line using a method substantially the same as that in Example 10 above, and the gene editing activity before and after the crRNA mutation was characterized by GFP fluorescence intensity by flow cytometry. As shown in Table 29.

TABLE 29
Designed mutants of DR sequences

| | | DR sequence |
|---|---|---|
| Grouping | DR sequence | (SEQ ID NO) |
| wt DR | gtaatgcgTctcccattg Acgc | 704 |
| DR-01 | gtaatgcgGctcccattgCcgc | 977 |
| DR-02 | gtaatgcgCctcccattgGcgc | 978 |
| DR-04 | gtaatgcgTctcccaGtgAcgc | 979 |
| DR-05 | gtaatgcgTctcccatGgAcgc | 980 |

The results show that the proportion of GFP-positive cells in each group is between 30% and 36%. The editing efficiency of DR-01 and DR-04 is slightly higher than that of wild-type DR. The editing efficiency of DR-02 and DR-05 is higher than that of DR-01 and DR-04.

### Example 20. Testing Editing Efficiency after Fusion with Different Domains

Theoretically, in some cases, the fusion of a nuclease binding domain to a specific protein may enhance the binding ability of the original protein to DNA or RNA, and the fusion of a nuclease cleavage domain can enhance or expand the cleavage ability of the original protein.

Therefore, in this example, the mutant version of C12-279, Mut-02-1-426-846-858-860 (C12-279-V4), was fused with each domain in Table 30, and the editing efficiency was tested in the Reporter 3 cell line.

TABLE 30
Fusion domains

| Domain name | SEQ ID NO |
|---|---|
| CHD2 | 981 |
| GSTM3 | 982 |
| OTUB1 | 983 |
| PHYHD1 | 984 |
| SCGN | 985 |
| SNCB | 986 |
| SSBP1 | 987 |
| SSO7d | 988 |
| TBCB | 989 |
| TIGER | 990 |
| topA | 991 |
| UCHL1 | 992 |
| YWHAQ | 993 |
| dRusA | 994 |
| albA | 995 |
| Cas2 | 996 |
| DBPJ | 997 |
| DDB1(S78E) | 998 |
| DG5P | 999 |
| hup | 1000 |
| SSDBP | 1001 |
| T5 | 1002 |

Take CHD2 as an example to illustrate: to construct a fusion protein expression plasmid, CHD2 was first codon optimized, a linker was added, and then gene synthesis was performed. The synthetic sequence is as follows:

>CHD2-Syn
(SEQ ID NO: 1003)
GGTGGAGGAGGTGGGTCTGGCGTGGAGGACGACAGCCGCCTGCTGCTGG
GCATCTACGAGCACGGCTACGGCAACTGGGAGCTGATCAAGACCGACCC
CGAGCTGAAGCTGACCGACAAGCTG.

The bold black sequence is the inserted linker, and the underlined sequence is the base sequence after CHD2 codon optimization.

Using the synthetic fragment CHD2-Syn as a template, CHD2-PF (SEQ ID NO: 1004) and CHD2-PR (SEQ ID NO: 1005) as amplification primer sequences, we obtained fragment CHD2-F by PCR amplification. The plasmid C12-279-V4 was digested with XhoI, and the 8945 bp vector fragment was recovered by gel. It was recombined with fragment CHD2-F in vitro (NEB™, Gibson Assembly® Master Mix) and transformed into Escherichia coli by heat shock to obtain the fusion protein expression plasmid C12-279-V4-CDH2 (SEQ ID NO: 1006).

The construction of other domain fusion proteins is exactly the same as CDH2 domain except that the fusion protein sequence is changed. The insertion position, insertion method and linker sequence are exactly the same. The Reporter 3 cell line was transfected and flow cytometry was performed in the same way. The editing efficiency was characterized by the ratio of the number of GFP-positive cells detected by flow cytometry. The results show that all fusion proteins have editing activity.

What is claimed is:

1. A non-naturally occurring Cas12 protein, wherein the Cas12 protein comprises the amino acid sequence having at least 80% sequence identity to the amino acid sequence shown in SEQ ID NO: 696, wherein the Cas12 protein has one or more mutations relative to the amino acid sequence shown in SEQ ID NO: 696.

2. The Cas12 protein of claim 1, wherein the Cas12 protein forms a complex with a guide polynucleotide, the guide polynucleotide comprises a guide sequence that is reverse complementary to a target sequence in a target nucleic acid, and the guide polynucleotide comprises a scaffold sequence that interacts with the Cas12 protein.

3. The Cas12 protein of claim 1, wherein the Cas12 protein has at least one mutation at one or more amino acid residues corresponding to positions 1, 2, 3, 4, 5, 7, 10, 24, 30, 48, 51, 55, 58, 59, 66, 108, 118, 138, 141, 175, 178, 185, 186, 257, 333, 352, 356, 375, 376, 378, 379, 383, 397, 400, 416, 426, 443, 449, 456, 459, 462, 469, 484, 485, 509, 561, 597, 607, 609, 623, 638, 639, 640, 697, 722, 731, 733, 755, 758, 771, 773, 779, 781, 784, 785, 786, 789, 792, 794, 798, 822, 823, 825, 826, 829, 830, 833, 834, 836, 842, 845, 846, 847, 850, 851, 853, 855, 856, 858, 859, 860, 866, 884, 892, 893, 900, 904, 926, 956, 985, 988, 989, 992, 993, 996, 1016, 1033, 1045, 1050, 1073, 1074, 1095, 1100, 1124, 1129, or 1132 of the amino acid sequence shown in SEQ ID NO: 696.

4. The Cas12 protein of claim 1, wherein the Cas12 protein has at least one mutation at one or more amino acid residues corresponding to positions 1, 2, 3, 4, 5, 6, 7, 8, 9, 10, 13, 14, 15, 16, 19, 21, 22, 23, 24, 25, 26, 27, 28, 30, 32, 33, 34, 37, 39, 41, 42, 43, 44, 46, 47, 48, 49, 50, 51, 54, 55, 56, 58, 59, 61, 62, 66, 67, 69, 70, 74, 76, 77, 78, 79, 80, 81, 82, 83, 84, 85, 86, 89, 90, 91, 92, 93, 101, 102, 103, 104, 105, 106, 108, 109, 110, 111, 112, 114, 115, 116, 117, 118, 119, 120, 121, 123, 124, 125, 126, 127, 128, 129, 130, 131, 132, 133, 134, 136, 138, 139, 140, 141, 142, 143, 144, 145, 146, 147, 151, 152, 154, 155, 157, 158, 159, 161, 162, 163, 165, 166, 169, 170, 175, 178, 180, 182, 183, 184, 185, 186, 187, 188, 191, 195, 196, 197, 200, 203, 205, 206, 209, 210, 212, 213, 214, 216, 217, 218, 219, 220, 221, 222, 223, 224, 225, 227, 229, 230, 231, 233, 234, 235, 237, 239, 240, 242, 243, 245, 247, 250, 251, 252, 255, 257, 259, 260, 263, 265, 266, 267, 268, 269, 271, 272, 273, 274, 276, 278, 281, 284, 285, 287, 288, 289, 290, 291, 293, 295, 296, 297, 299, 301, 303, 305, 306, 308, 309, 310, 313, 315, 316, 317, 318, 319, 321, 322, 323, 325, 327, 329, 330, 331, 333, 334, 335, 337, 340, 341, 342, 344, 345, 346, 347, 348, 350, 351, 352, 353, 354, 355, 356, 357, 358, 359, 360, 361, 362, 363, 364, 365, 368, 369, 370, 371, 372, 375, 376, 377, 378, 379, 380, 381, 382, 383, 385, 386, 387, 388, 390, 391, 392, 394, 396, 397, 398, 399, 400, 401, 402, 403, 404, 405, 406, 407, 408, 409, 410, 411, 412, 413, 414, 416, 417, 418, 419, 420, 421, 422, 423, 424, 425, 426, 427, 428, 429, 431, 433, 435, 437, 439, 441, 442, 443, 444, 446, 447, 448, 449, 450, 451, 452, 454, 455, 456, 459, 461, 462, 463, 464, 467, 469, 470, 473, 475, 476, 477, 478, 479, 480, 481, 482, 483, 484, 485, 486, 487, 488, 489, 490, 491, 492, 493, 494, 496, 497, 499, 500, 501, 502, 503, 504, 507, 509, 511, 512, 513, 514, 515, 516, 517, 518, 520, 521, 522, 524, 525, 527, 529, 532, 533, 535, 536, 537, 538, 539, 540, 541, 542, 545, 546, 547, 548, 549, 552, 553, 555, 556, 557, 558, 559, 560, 561, 562, 563, 564, 565, 566, 567, 568, 569, 572, 574, 575, 576, 577, 578, 579, 580, 581, 582, 583, 584, 585, 586, 587, 589, 590, 593, 595, 597, 598, 599, 601, 602, 603, 604, 605, 606, 607, 608, 609, 610, 611, 612, 614, 618, 620, 621, 623, 625, 627, 628, 630, 631, 632, 633, 634, 635, 636, 637, 638, 639, 640, 641, 642, 643, 644, 645, 646, 647, 653, 654, 655, 656, 657, 660, 662, 664, 665, 666, 667, 668, 669, 670, 671, 672, 673, 674, 675, 676, 678, 679, 680, 683, 685, 688, 692, 694, 695, 696, 697, 698, 699, 700, 701, 705, 706, 707, 709, 710, 711, 712, 713, 715, 716, 717, 719, 720, 721, 722, 723, 724, 725, 727, 728, 729, 730, 731, 732, 733, 734, 736, 737, 738, 739, 740, 741, 742, 743, 745, 746, 747, 748, 749, 751, 752, 753, 754, 755, 756, 758, 759, 760, 762, 764, 765, 766, 767, 768, 769, 771, 773, 774, 775, 776, 779, 780, 781, 782, 784, 785, 786, 789, 790, 792, 794, 795, 797, 798, 801, 802, 804, 805, 808, 809, 810, 811, 812, 813, 814, 815, 817, 818, 819, 821, 822, 823, 824, 825, 826, 827, 828, 829, 830, 831, 832, 833, 834, 835, 836, 837, 838, 839, 840, 842, 844, 845, 846, 847, 848, 850, 851, 852, 853, 855, 856, 857, 858, 859, 860, 862, 863, 864, 865, 866, 868, 870, 873, 874, 875, 877, 879, 880, 881, 882, 883, 884, 886, 887, 892, 893, 895, 896, 897, 898, 899, 900, 901, 902, 903, 904, 905, 909, 910, 913, 914, 916, 917, 918, 919, 920, 921, 922, 923, 924, 926, 928, 930, 931, 932, 933, 934, 936, 938, 939, 947, 949, 950, 951, 952, 953, 954, 955, 956, 957, 958, 959, 960, 964, 965, 966, 967, 969, 970, 972, 973, 974, 975, 976, 977, 980, 981, 982, 985, 986, 988, 989, 990, 991, 992, 993, 994, 995, 996, 997, 1001, 1005, 1007, 1009, 1010, 1011, 1012, 1014, 1016, 1017, 1018, 1021, 1023, 1024, 1025, 1026, 1027, 1028, 1029, 1030, 1032, 1033, 1036, 1037, 1038, 1039, 1040, 1041, 1043, 1044, 1045, 1047, 1050, 1055, 1059, 1060, 1061, 1064, 1065, 1066, 1067, 1068, 1069, 1070, 1071, 1072, 1073, 1074, 1075, 1076, 1077, 1078, 1080, 1092, 1093, 1094, 1095, 1096, 1097, 1098, 1100, 1101, 1102, 1103, 1104, 1105, 1107, 1108, 1109, 1110, 1111, 1112, 1113, 1115, 1116, 1117, 1120, 1122, 1123, 1124, 1125, 1128, 1129, 1130, 1131, 1132, 1133, 1134, or 1135 of the amino acid sequence shown in SEQ ID NO: 696.

5. The Cas12 protein of claim 1, wherein the Cas12 protein has at least one mutation at one or more amino acid residues corresponding to positions selected from I33, G184, S185, Q186, G194, N195, G196, G197, N245, G256, L260, Y278, S285, Y316, H350, D352, A355, A356, C385, P386, H387, G390, K391, N392, D429, Q461, Q462, Q469, E485, S491, K521, P525, L611, K629, K631, N633, D841, N898, K987, A988, G989, Q990, T991, D1010, E1013, A1136, K1138, or T1139 of the amino acid sequence shown in SEQ ID NO: 696.

6. The Cas12 protein of claim 1, wherein the Cas12 protein has mutations at amino acid residues corresponding to a combination of positions of SEQ ID NO: 696, wherein the combination of positions is selected from the group consisting of: 186+352+1+426+846+858+860, 186+352+1+426+846+860, 186+352+1+5+426+858+860, 186+352+1+3+426+858+860, 186+352+3+426+860, 186+352+1+333+426+858+860, 186+352+1+426+485+858+860, 186+352+1+5+426+860, 186+352+3+426+858+860, 186+352+5+426+858+860, 186+352+333+426+858+860, 186+352+333+426+860, 186+352+426+846+860, 186+352+5+426+860, 186+352+1+3+426+860, 5+426+860, 186+352+426+846+858+860, 186+352+1+426+485+860, 426+858+860, 7+426+858, 186+352+426+860, 186+352+426+485+858+860, 186+352+426+485+860, 426+846+858+860, 7+426+846, 186+352+860, 184+186+352+376+1132, 5+333+426, 5+426+858, 186+352+1+333+426+860, 184+186+352+3+107+426, 186+352+5+426, 333+376+426, 186+352+5, 2+5+846+858, 186+352+3+426, 5+846+858, 186+352+426+846, 186+352+7, 186+352+3+376+426, 186+352+376+426+860+865, 186+352+376+426, 3+846+860, 846+858+988, 3+426+858, 3+846+858+860, 3+858+860, 186+352+858, 184+186+352+860, 333+426, 184+186+352+3+376, 3+426+860, 846+860+988, 3+860, 184+186+352+3+639, 186+352+333+426, 846+858+860, 186+352+426, 333+426+846, 333+426+485, 5+846+860, 3+846+988, 184+186+352+376, 3+333+426, 186+352+426+485, 333+426+858, 333+426+1132, 428+485, 186+352+333+376+426, 858+860, 186+352+376+426+485+860, 184+186+352+426, 186+352+639, 5+858+988, 3+858+988, 5+858, 3+858, 184+186+352+846, 184+186+352+639, 858+988, 184+186+352+426+1132, 186+352+1132, 184+186+352+5, 184+186+352+858, 858+860+1132, 3+5, 426+649, 186+352+426+485+860, 186+352+333, 184+186+352, 186+376, 846+860, 858+988+1132, 846+858, 333+376, 376+426, 184+186+352+3, 3+846+1132, 5+846+1132, 186+352+426+1132, 376+426+485+660, 426, 5+846, 846+860+1132, 333+376+485, 184+186+352+639+1132, 352+426, 333+485, 184+186+352+333, 846+858+1132, 333+426+860, 186+352+988, 5+860, 846+988, 186+352, 3+846, 846+1132, 184+186+352+1132, 186+485, 988+1132, 184+186+352+485, 376+485, 5+1132, 3+7, 186+352+485, 184+186+352+7, 184+186+352+333+336, 3+1132, 426+858+988, 186+352+376, 186+352+3, and 186+352+333+336+376+426.

7. A nuclease-inactivated mutant of the Cas12 protein of claim 1, wherein an inactivating mutation of the nuclease-inactivated mutant is selected from one or more of D651A, E891A, and D1082A at positions corresponding to the amino acid sequence shown in SEQ ID NO: 696.

8. A fusion protein or conjugate, comprising:
(1) a Cas12 protein, wherein the Cas12 protein comprises an amino acid sequence having at least 80% sequence identity to the amino acid sequence shown in SEQ ID NO: 696; and
(2) a homologous or heterologous functional domain.

9. The fusion protein or conjugate of claim 8, wherein the homologous or heterologous functional domain is selected from one or more of the following: a subcellular positioning signal, a DNA binding domain, a protease domain, a transcriptional activation domain, a transcriptional repression domain, a nuclease domain, a deaminase domain, a uracil DNA glycosylase domain (UDG), a uracil DNA glycosylase inhibitory domain (UGI), a DNA methyltransferase, a DNA demethylase, a histone methyltransferase, a histone demethylase, a transcription release factor, a histone acetylase domain, a histone deacetylase domain, a DNA ligase, an affinity tag, a reporter tag, an affinity domain, and a reporter domain.

10. The fusion protein or conjugate of claim 9, wherein the subcellular positioning signal is selected from the group consisting of a nuclear localization signal, a nuclear export signal, a mitochondrial localization signal, and a chloroplast localization signal.

11. An isolated nucleic acid encoding the fusion protein of claim 8.

12. A CRISPR-Cas12 system, comprising:
a. a fusion protein comprising a Cas12 protein, wherein the Cas12 protein comprises an amino acid sequence having at least 80% sequence identity to the amino acid sequence shown in SEQ ID NO: 696, or an isolated nucleic acid encoding the fusion protein; and
b. a guide polynucleotide, or a polynucleotide sequence encoding the guide polynucleotide;
wherein the fusion protein forms a complex with the guide polynucleotide; and the guide polynucleotide comprises a guide sequence engineered to guide a sequence-specific binding of the complex to a target nucleic acid.

13. The CRISPR-Cas12 system of claim 12, wherein the target nucleic acid is a gene selected from the genes listed in Table 27.

14. A vector system comprising one or more recombinant vectors, wherein at least one recombinant vector of the one or more recombinant vectors comprises the fusion protein of claim 12 and a polynucleotide sequence encoding the guide polynucleotide.

15. A cell, comprising the CRISPR-Cas12 system of claim 12.

16. The cell of claim 15, wherein the cell is a human cell.

17. A kit, comprising the Cas12 protein of claim 1.

18. A method for detecting, binding, or cleaving a target nucleic acid, comprising: contacting the target nucleic acid with the Cas12 protein of claim 1.

19. A method for diagnosing, treating, or preventing a disease or disorder associated with a target nucleic acid, comprising: applying the CRISPR-Cas12 system of claim 12 to a sample from a subject in need or the subject in need.

20. The method of claim 19, wherein the target nucleic acid is selected from genes as listed in Table 27, and the disease or disorder is the disease or disorder as listed in Table 27.
